(19) United States
(12) Patent Application Publication
(10) Pub. No.: US 2022/0387628 A1
(43) Pub. Date: Dec. 8, 2022

(54) MESSENGER RNA COMPRISING FUNCTIONAL RNA ELEMENTS AND USES THEREOF

(71) Applicant: ModernaTX, Inc., Cambridge, MA (US)

(72) Inventors: Ruchi JAIN, Brookline, MA (US); David REID, Somerville, MA (US); Vladimir PRESNYAK, Manchester, NH (US); Alicia BICKNELL, Cambridge, MA (US); Caroline KÖHRER, Cambridge, MA (US)

(21) Appl. No.: 17/618,779

(22) PCT Filed: Jun. 24, 2020
(86) PCT No.: PCT/US2020/039365

§ 371 Date: Dec. 13, 2021

Related U.S. Application Data
(60) Provisional application No. 62/865,811, filed on Jun. 24, 2019.

(51) Int. Cl. A61K 48/00

(52) U.S. Cl. CPC A61K 48/0066

(57) ABSTRACT

The present disclosure provides messenger RNAs (mRNAs) having chemical and/or structural modifications, including RNA elements and/or modified nucleotides, which provide desirable regulation of mRNA localization, stability, and/or translation to yield increased mRNA expression and activity of an encoded polypeptide.

### CROSS-REFERENCE TO RELATED APPLICATIONS

This application claims the benefit of U.S. Provisional Patent Application Ser. No. 62/865,811, filed Jun. 24, 2019. The entire contents of which is incorporated herein by reference.

### BACKGROUND

Administration of a synthetic and/or in vitro-generated mRNA that structurally resembles natural mRNA can result in the controlled production of therapeutic proteins or peptides via the endogenous and constitutively-active translation machinery (e.g. ribosomes) that exists within a patient's own cells. In recent years, the development and use of mRNA as a therapeutic agent has demonstrated potential for treatment of numerous diseases and for the development of novel approaches in regenerative medicine and vaccination (Stanton et al (2017) Messenger RNA as a Novel Therapeutic Approach. In: Garnder A. (eds) RNA Therapeutics. Topics in Medicinal Chemistry, vol 27 Springer, Cham; Sabnis et al. (2018) Mol Ther 26:1509-1519; Hassett et al. (2019) Mol Ther Nucleic Acids 15:P1-11).

In eukaryotic cells, the amount of expressed protein is often only weakly correlated with the amount of the corresponding mRNA that is transcribed. Instead, the level of protein expression is strongly dependent upon post-transcriptional steps, including processes that regulate mRNA stability, localization and translation. Functional RNA elements contained in an mRNA, including specific RNA sequences or RNA structural motifs that are located in the untranslated regions (e.g., the 5′ or 3′ UTRs) and/or coding regions of the mRNA, can affect its stability, translation and sequestration to certain cellular compartments.

It is recognized that the control and regulation of mRNA stability, cellular localization, and translation is an important development component in order for mRNA drugs to achieve a desired therapeutic effect. There exists a need to develop mRNAs with improved therapeutic effect.

### SUMMARY OF THE INVENTION

Improving the expression level and/or activity of a therapeutic polypeptide encoded by an mRNA is a desirable outcome in the development of mRNA therapeutics. The present disclosure is based, at least in part, on the discovery of chemical and/or structural modifications that provide increased mRNA expression level and/or activity of an encoded translation product (e.g., an encoded polypeptide of interest). These include RNA elements (e.g., specific RNA sequences or RNA structural motifs) that regulate the post-transcriptional stability, localization, and/or translation of an mRNA, thereby yielding improved mRNA expression and/or activity of an encoded polypeptide of interest. Without being bound by theory, RNA elements of the disclosure are expected to increase mRNA expression level and/or activity by regulating the post-transcriptional stability of an mRNA and increasing mRNA half-life, for example, by protecting the mRNA from degradation. Additionally, in some embodiments, RNA elements of the disclosure are expected to direct localization of the mRNA to certain subcellular compartments, thus improving mRNA expression and/or activity by localizing the mRNA to regions of the cell that promote translation (e.g., by localization to membrane-associated ribosomes of the mitochondria and/or the endoplasmic reticulum) and/or regions that promote increased mRNA half-life (e.g., localization to regions with reduced exposure to endogenous exonuclease and/or endonuclease activity). While, in some embodiments, RNA elements of the disclosure yield increased mRNA expression level and/or activity by performing one or more desired translational regulatory activities that modulate (e.g., control) translation of an mRNA to produce a desired translational product, for example by promoting translation of only one open reading frame (ORF) encoding a desired polypeptide of interest.

Thus, in some aspects, the mRNAs of the disclosure comprise certain RNA elements that regulate post-transcriptional mRNA stability, localization or perform a desired translational regulatory activity, thereby resulting in increased mRNA expression level and/or activity of an encoded polypeptide. In some aspects, the RNA elements are located in the 5′ untranslated region (UTR) and/or the 3′UTR of the mRNA.

In some aspects, one or more RNA elements in the 5′UTR perform a desired translational regulatory activity that modulates (e.g., controls) the translation of an mRNA to produce a desired translational product. In some embodiments, one or more RNA elements in the 5′UTR reduces, inhibits or eliminates the failure to initiate translation of the therapeutic protein or peptide at the desired initiator codon, which otherwise may occur as a consequence of leaky scanning or other mechanisms. Leaky scanning can result in the bypass of the desired initiation codon that begins the ORF encoding a polypeptide of interest or a translation product. This bypass can further result in the initiation of polypeptide synthesis from an alternate or alternative initiation codon, and thereby promote the translation of partial, aberrant, or otherwise undesirable open reading frames within the mRNA. The negative impact caused by the failure to initiate translation of the therapeutic protein or peptide at the desired initiator codon, as a consequence of leaky scanning or other mechanisms, poses a challenge in the development of mRNA therapeutics.

In one aspect, the present disclosure is based, at least in part, on the discovery that mRNAs having a 5′UTR that comprises one or more functional RNA elements (e.g., an RNAse P stem loop derived from the RNAse P ribonucleoprotein), gives rise to initiation at a first AUG codon that begins an ORF encoding a desired polypeptide of interest. When incorporated into the 5′UTR of an mRNA, an RNAse P stem loop results in up to 85% reduction in leaky scanning relative to an mRNA lacking the RNAse P stem loop. Additionally, it was discovered that mRNA encoding a cellular enzyme having a 5′UTR comprising an RNAse P stem loop gives increased expression and activity of the encoded polypeptide relative to an mRNA lacking the RNAse P stem loop. Accordingly, the present disclosure provides mRNAs having a 5′UTR comprising an RNAse P stem loop which provides a desired translational regulatory activity and results in increased mRNA expression and activity of an encoded polypeptide of interest.

In another aspect, the present disclosure is based, at least in part, on the discovery that mRNAs having a 3′UTR that is derived from an mRNA encoding a nuclear encoded mitochondrial protein (NEMP) gives increased mRNA expression and/or activity of an encoded polypeptide relative to mRNAs that do not have such 3′UTRs both in vitro and in vivo. Without being bound by theory, it is believed that 3′UTRs derived from naturally-occurring mRNAs that encoded NEMPs, such as those described herein, comprise RNA elements that regulate the stability, localization, and translation of the mRNA. Further, and without being bound by theory, it is believed that various proteins within the cell are able to recognize these RNA elements and function to sort the mRNA to certain subcellular compartments, promote the stability of the mRNA, and/or promote the translation of the mRNA.

Surprisingly, it was discovered that delivery of a lipid nanoparticle comprising a modified mRNA encoding a cellular enzyme and comprising a heterologous 3′UTR having a nucleotide sequence that is substantially identical to the nucleotide sequence of a 3′UTR from an mRNA encoding a NEMP (e.g., a NEMP-derived 3′UTR) resulted in higher expression and activity of the encoded protein in vivo relative to an mRNA that did not comprise the 3′UTR. It was further discovered that the treatment, when administered to mice deficient in the cellular enzyme, resulted in a decrease in biomarkers that are abnormally high under conditions of enzyme-deficiency.

Additionally, it was discovered that modified mRNAs comprising a combination of a heterologous NEMP-derived 3′UTR and a 5′UTR comprising functional RNA elements (e.g., an RNAse P stem loop) resulted in increased expression level of an encoded polypeptide in vitro. The increased expression of an encoded polypeptide as a result of combining a NEMP-derived 3′UTR and a 5′UTR comprising functional RNA elements (e.g., an RNAse P stem loop) was consistent for mRNAs encoding a cellular enzyme, an intracellular protein, or a secreted protein. Furthermore, treatment with a lipid nanoparticle comprising a modified mRNA encoding a cellular enzyme and combining a NEMP-derived 3′UTR and a 5′UTR comprising functional RNA elements (e.g., an RNAse P stem loop) resulted in increased expression and enzymatic activity of the encoded cellular enzyme when administered in vivo. Without being bound by theory, it is believed that enhanced expression and activity of the encoded protein in vivo occurs due to post-transcriptional regulation of mRNA stability, localization, and/or translation efficiency resulting from chemical and/or structural modification of the mRNA.

Accordingly, the present disclosure provides mRNAs (e.g., modified mRNAs) encoding a polypeptide of interest and comprising a heterologous NEMP-derived 3′UTR, a 5′UTR comprising functional RNA elements (e.g., an RNAse P stem loop), or a combination thereof that enhance protein expression and/or activity, as well as compositions (e.g., lipid nanoparticles) and methods thereof (e.g., methods for treating a disease that would benefit from increased expression of a therapeutic polypeptide such as cancer, an autoimmune disease, an infectious disease, a metabolic disease).

In some aspects, the present disclosure provides a messenger RNA (mRNA), wherein the mRNA comprises: a 5′ cap, a 5′ untranslated region (UTR) comprising a structural RNA element comprising a stem-loop, an initiation codon, a full open reading frame encoding a polypeptide, and a 3′ UTR, wherein the structural RNA element comprises a sequence of linked nucleotides, wherein each nucleotide comprises a nucleobase selected from the group consisting of: adenine, guanine, thymine, uracil, and cytosine, or derivatives or analogs thereof, and wherein the structural RNA element provides a translational regulatory activity selected from:
a. increasing residence time of a 43S pre-initiation complex (PIC) or ribosome at, or proximal to, the initiation codon;
b. increasing initiation of polypeptide synthesis at or from the initiation codon;
c. increasing an amount of polypeptide translated from the full open reading frame;
d. increasing fidelity of initiation codon decoding by the PIC or ribosome;
e. inhibiting or reducing leaky scanning by the PIC or ribosome;
f. decreasing a rate of decoding the initiation codon by the PIC or ribosome;
g. inhibiting or reducing initiation of polypeptide synthesis at any codon within the mRNA other than the initiation codon;
h. inhibiting or reducing the amount of polypeptide translated from any open reading frame within the mRNA other than the full open reading frame;
i. inhibiting or reducing the production of aberrant translation products;
j. increasing ribosomal density on the mRNA; and
k. a combination of any of (a)-(j).

In any of the foregoing aspects, the structural RNA element comprises a nucleotide sequence of about 10-30 nucleotides, about 15-25 nucleotides, or about 20-25 nucleotides. In some aspects, the structural RNA element comprises a nucleotide sequence of about 15-25 nucleotides.

In any of the foregoing aspects, the structural RNA element comprises a nucleotide sequence of about 30, 29, 28, 27, 26, 25, 24, 23, 22, 21, 20, 19, 18, 17, 16, 15, 14, 13, 12, or about 10 nucleotides in length.

In any of the foregoing aspects, the structural RNA element comprises a double-stranded stem comprising about 3-8 base pairs, about 4-7 base pairs, about 5-6 base pairs, or about 3, 4, 5, 6, 7, or 8 base pairs. In some aspects, the double-stranded stem comprises about 4-7 base pairs. In some aspects, the double-stranded stem comprises at least 50% G/C base pairs. In some aspects, the double-stranded stem comprises at least 60%, at least 65%, at least 70%, at least 75%, at least 80%, at least 85%, or at least 90% G/C base pairs. In some aspects, the double-stranded stem comprises 30% or less A/U base pairs.

In any of the foregoing aspects, the structural RNA element comprises a stem-loop comprising a single-stranded loop of about 3-8 nucleotides, about 4-7 nucleotides, about 5-6 nucleotides, about 3, 4, 5, 6, 7, or 8 nucleotides in length. In some aspects, the single-stranded loop is about 4-7 nucleotides in length.

In any of the foregoing aspects, the structural RNA element comprises at least 50%, at least 60%, at least 65%, at least 70%, at least 75%, at least 80%, at least 85%, or at least 90% G/C bases. In some aspects, the structural RNA element comprises at least 60% G/C bases. In some aspects, the structural RNA element comprises 40% or less A/U bases.

In any of the foregoing aspects, the mRNA comprises a structural RNA element comprises a nucleotide sequence at least 80%, 85%, 90%, 95%, 96%, 97%, 98%, or 99% identical to the nucleotide sequence of SEQ ID NO: 6. In some aspects, the mRNA comprises a structural RNA element comprising a nucleotide sequence which differs from SEQ ID NO: 6 by substitution, deletion, or insertion of 1, 2, 3, 4, or 5 nucleotides. In some aspects, the structural RNA element comprises at least 50%, at least 60%, at least 65%, at least 70%, at least 75%, at least 80%, at least 85%, or at least 90% G/C bases.

In any of the foregoing aspects, the mRNA comprises a structural RNA element that comprises a double-stranded stem of about 4-7 base pairs and a nucleotide sequence which differs from SEQ ID NO: 6 by substitution, deletion or insertion of 1, 2, 3, 4, or 5 nucleotides. In some aspects, the mRNA comprises a structural RNA element that comprises a single-stranded loop of about 4-7 bases and a nucleotide sequence which differs from SEQ ID NO: 6 by substitution, deletion or insertion of 1, 2, 3, 4, or 5 nucleotides. In some aspects, the structural RNA element comprises at least 50%, at least 60%, at least 65%, at least 70%, at least 75%, at least 80%, at least 85%, or at least 90% G/C bases.

In any of the foregoing aspects, the mRNA comprises a structural RNA element that comprises a nucleotide sequence at least 80%, 85%, 90%, 95%, 96%, 97%, 98%, or 99% identical to the nucleotide sequence of SEQ ID NO: 47. In some aspects, the mRNA comprises a structural RNA element that comprises a nucleotide sequence which differs from SEQ ID NO: 47 by substitution, deletion or insertion of 1, 2, 3, 4, or 5 nucleotides. In some aspects, the structural RNA element comprises at least 50%, at least 60%, at least 65%, at least 70%, at least 75%, at least 80%, at least 85%, or at least 90% G/C bases.

In any of the foregoing aspects, the mRNA comprises a structural RNA element that comprises a double-stranded stem of about 4-7 base pairs and a nucleotide sequence which differs from SEQ ID NO: 47 by substitution, deletion or insertion of 1, 2, 3, 4, or 5 nucleotides. In some aspects, the mRNA comprises a structural RNA element that comprises single-stranded loop of about 4-7 bases and a nucleotide sequence which differs from SEQ ID NO: 47 by substitution, deletion or insertion of 1, 2, 3, 4, or 5 nucleotides. In some aspects, the structural RNA element comprises at least 50%, at least 60%, at least 65%, at least 70%, at least 75%, at least 80%, at least 85%, or at least 90% G/C bases.

In any of the foregoing aspects, the mRNA comprises a structural RNA element has a deltaG (ΔG) of about −20 to −30 kcal/mol, about −20 to −25 kcal/mol, about −15 to −20 kcal/mol, about −10 to −15 kcal/mol, or about −5 to −10 kcal/mol.

In some aspects, the present disclosure provides an mRNA, wherein the mRNA comprises: a 5′ cap, a 5′ UTR comprising a structural RNA element comprising a stem-loop, an ORF encoding a polypeptide, and a 3′ UTR, wherein the structural RNA element comprises a sequence of 15-25 linked nucleotides, wherein each nucleotide comprises a nucleobase selected from the group consisting of: adenine, guanine, thymine, uracil, and cytosine, or derivatives or analogs thereof, and wherein the structural RNA element comprises (i) a double-stranded stem of about 4-7 base pairs comprising at least 50% G/C base pairs; (ii) a single-stranded loop of about 3-8 nucleotides; and (iii) a deltaG (ΔG) about −10 to −15 kcal/mol. In some aspects, the double-stranded stem comprises at least 60%, at least 65%, at least 70%, at least 75%, at least 80%, at least 85%, or at least 90% G/C base pairs. In some aspects, the double-stranded stem comprises 30% or less A/U base pairs. In some aspects, the single-stranded loop is about 4-7 nucleotides in length. In some aspects, the structural RNA element comprises at least 50%, at least 60%, at least 65%, at least 70%, at least 75%, at least 80%, at least 85%, or at least 90% G/C bases. In some aspects, the structural RNA element comprises at least 60% G/C bases. In some aspects, the structural RNA element comprises 40% or less A/U bases.

In some aspects, the present disclosure provides an mRNA, wherein the mRNA comprises: a 5′ cap, a 5′ UTR comprising a structural RNA element comprising a stem-loop, an ORF encoding a polypeptide, and a 3′ UTR, wherein the structural RNA element comprises a sequence of 15-25 linked nucleotides, wherein each nucleotide comprises a nucleobase selected from the group consisting of: adenine, guanine, thymine, uracil, and cytosine, or derivatives or analogs thereof, and wherein the structural RNA element comprises (i) a double-stranded stem of about 4-7 base pairs; (ii) a single-stranded loop of about 3-8 nucleotides; (iii) a nucleotide sequence which differs from SEQ ID NO: 6 or SEQ ID NO: 47 by substitution, deletion or insertion of 1, 2, 3, 4, or 5 nucleotides; and (iv) a deltaG (ΔG) about −10 to −15 kcal/mol. In some aspects, the double-stranded stem comprises at least 60%, at least 65%, at least 70%, at least 75%, at least 80%, at least 85%, or at least 90% G/C base pairs. In some aspects, the double-stranded stem comprises 30% or less A/U base pairs. In some aspects, the single-stranded loop is about 4-7 nucleotides in length. In some aspects, the structural RNA element comprises at least 50%, at least 60%, at least 65%, at least 70%, at least 75%, at least 80%, at least 85%, or at least 90% G/C bases. In some aspects, the structural RNA element comprises at least 60% G/C bases. In some aspects, the structural RNA element comprises 40% or less A/U bases.

In some aspects, the disclosure provides an mRNA, wherein the mRNA comprises: a 5′ cap, a 5′ UTR comprising a structural RNA element comprising a stem-loop, an ORF encoding a polypeptide, and a 3′ UTR, wherein the structural RNA element comprises (i) a nucleotide sequence at least 80%, 85%, 90%, 95%, 96%, 97%, 98%, or 99% identical to the nucleotide sequence of SEQ ID NO: 6 or the nucleotide sequence of SEQ ID NO: 47.

In any of the foregoing aspects, the mRNA comprises a structural RNA element comprising a nucleotide sequence at least 80%, 85%, 90%, 95%, 96%, 97%, 98%, or 99% identical to the nucleotide sequence of SEQ ID NO: 6. In some aspects, the structural RNA element has a deltaG (ΔG) of about −20 to −25 kcal/mol, about −15 to −20 kcal/mol, or about −10 to −15 kcal/mol. In some aspects, the structural RNA element has a deltaG (ΔG) about −10 to −15 kcal/mol.

In any of the foregoing aspects, the mRNA comprises a structural RNA element comprising a nucleotide sequence at least 80%, 85%, 90%, 95%, 96%, 97%, 98%, or 99% identical to the nucleotide sequence of SEQ ID NO: 47. In some aspects, the structural RNA element has a deltaG (ΔG) of about −20 to −25 kcal/mol, about −15 to −20 kcal/mol, or about −10 to −15 kcal/mol. In some aspects, the structural RNA element has a deltaG (ΔG) about −10 to −15 kcal/mol.

In any of the foregoing aspects, the mRNA comprises a structural RNA element, wherein the structural RNA element provides a translational regulatory activity comprising increasing an amount of polypeptide translated from the full open reading frame.

In some aspects, the disclosure provides an mRNA, wherein the mRNA comprises: a 5′ cap, a 5′ UTR comprising a structural RNA element comprising the nucleotide sequence of SEQ ID NO: 6, an ORF encoding a polypeptide, and a 3′ UTR.

In some aspects, the disclosure provides an mRNA, wherein the mRNA comprises: a 5′ cap, a 5′ UTR comprising a structural RNA element comprising the nucleotide sequence of SEQ ID NO: 47, an ORF encoding a polypeptide, and a 3′ UTR.

In any of the foregoing aspects, the 5′ UTR comprises a Kozak-like sequence upstream of the initiation codon and the structural RNA element is located upstream of the Kozak-like sequence in the 5′ UTR. In some aspects, the 5′UTR comprises a structural RNA element that is located about 45-50, about 40-45, about 35-40, about 30-35, about 25-30, about 20-25, about 15-20, about 10-15, about 6-10 nucleotides, about 1-5 nucleotides, or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide upstream of the Kozak-like sequence in the 5′ UTR. In some aspects, the structural RNA element is located about 40-45 nucleotides upstream of the Kozak-like sequence in the 5′ UTR. In some aspects, the structural RNA element is located about 10-15 nucleotides upstream of the Kozak-like sequence in the 5′ UTR. In some aspects, the structural RNA element is located about 6-10 nucleotides upstream of the Kozak-like sequence in the 5′ UTR.

In any of the foregoing aspects, the structural RNA element is located downstream of the 5′ cap or 5′ end of the mRNA in the 5′ UTR. In some aspects, the structural RNA element is located about 45-50, about 40-45, about 35-40, about 30-35, about 25-30, about 20-25, about 15-20, about 10-15, about 5-10 nucleotides, about 1-5 nucleotide(s), or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) downstream of the 5′ cap or 5′ end of the mRNA in the 5′ UTR. In some aspects, the structural RNA element is located about 40-45 nucleotides downstream of the 5′ cap or 5′ end of the mRNA in the 5′ UTR. In some aspects, the structural RNA element is located about 20-25 nucleotides downstream of the 5′ cap or 5′ end of the mRNA in the 5′ UTR. In some aspects, the structural RNA element is located about 5-10 nucleotides downstream of the 5′ cap or 5′ end of the mRNA in the 5′ UTR.

In any of the foregoing aspects, the mRNA comprises a Kozak-like sequence in the 5′UTR, wherein the 5′UTR comprises a GC-rich RNA element comprising a sequence of about 20-30, about 10-20, about 10-15, about 5-15, or about 3-15 nucleotides, or derivatives or analogs thereof, wherein the sequence is at least about 50% cytosine, and wherein the GC-rich RNA element is located upstream of the Kozak-like in the 5′ UTR. In some aspects, the GC-rich RNA element comprises a sequence of about 3-15, about 15, 14, 13, 12, 11, 10, 9, 8, 7, 6, 5, 4, or 3 nucleotides, or derivatives or analogs thereof, wherein the sequence is about 50%-60% cytosine, about 60%-70% cytosine, or about 70%-80% cytosine. In some aspects, the GC-rich RNA element comprises a sequence of cytosine and guanine. In some aspects, the GC-rich RNA element comprises a sequence of about 3-30 guanine and cytosine nucleotides, or derivatives or analogues thereof, wherein the sequence comprises a repeating GC-motif, wherein the repeating GC-motif is [CCG]ₙ or [GCC]ₙ, wherein n=1 to 10, 1-5, 3, 2 or 1. In some aspects, the sequence of the GC-rich RNA element is selected from (i) the sequence of EK1 [CCCGCC] set forth in SEQ ID NO: 3; (ii) the sequence of EK2 [GCCGCC] set forth in SEQ ID NO: 18; and (iii) the sequence of EK3 [CCGCCG] set forth in SEQ ID NO: 19. In some aspects, the sequence of the GC-rich RNA element comprises the sequence of V1 [CCCCGGCGCC] set forth in SEQ ID NO: 1. In some aspects, the sequence of the GC-rich RNA element comprises the sequence of V2 [CCCCGGC] set forth in SEQ ID NO: 2. In some aspects, the sequence of the GC-rich RNA element comprises the sequence of CG1 [GCGCCCCGCGGCGCCCCGCG] set forth in SEQ ID NO: 20. In some aspects, the sequence of the GC-rich RNA element comprises the sequence of CG2 [CCCGCCCGCCCCGCCCCGCC] set forth in SEQ ID NO: 21.

In any of the foregoing aspects, the mRNA comprises a GC-rich RNA element that is located about 20-30, about 15-20, about 10-15, about 5-10, or about 1-5 nucleotides upstream of the Kozak-like sequence in the 5′ UTR. In some aspects, the GC-rich RNA element is located about 5, about 4, about 3, about 2, or 1 nucleotide(s) upstream of the Kozak-like sequence in the 5′ UTR. In some aspects, the GC-rich RNA element is upstream of and immediately adjacent to the Kozak-like sequence in the 5′ UTR. In some aspects, the Kozak-like sequence comprises the sequence [5′-GCCACC-′3] set forth in SEQ ID NO: 17 or [5′-GCCGCC-′3] set forth in SEQ ID NO: 48.

In some aspects, the mRNA comprises a GC-rich RNA element that comprises a stable RNA secondary structure located downstream of the initiation codon. In some aspects, the stable RNA secondary structure is a hairpin or a stem-loop. In some aspects, the stable RNA secondary structure has a deltaG of about −20 to −30 kcal/mol, about −10 to −20 kcal/mol, or about −5 to −10 kcal/mol. In some aspects, the GC-rich RNA element comprises a stable RNA secondary structure selected from (i) the sequence of SL1 [CCGCGGCGCCCCGCGG] as set forth in SEQ ID NO: 24; (ii) the sequence of SL2 [GCGCGCAUAUAGCGCGC] as set forth in SEQ ID NO: 25; (iii) the sequence of SL3 [CAUGGUGGCGGCCCGCCGCCACCAUG] as set forth in SEQ ID NO: 49; (iv) the sequence of SL4 [CAUGGUGGCCCGCCGCCACCAUG] as set forth in SEQ ID NO: 50; and (v) the sequence of SL5 [CAUGGUGCCCGCCGCCACCAUG] as set forth in SEQ ID NO: 51.

In any of the foregoing aspects, an mRNA comprises a GC-rich RNA element that is located about 20-30, about 10-20, about 15-20, about 10-15, about 5-10, or about 1-5 nucleotides downstream of the initiation codon.

In any of the foregoing aspects, an mRNA comprises a C-rich RNA element that is located proximal to the 5′ cap, wherein the C-rich RNA element comprises a sequence of about 3-20 nucleotides, wherein the sequence comprises about 50-55%, 55-60%, 60-65%, 70-75%, 75-80%, 80-85%, 85-90% or 90-95%, or about 95%, about 90%, about 85%, about 80%, about 75%, about 70%, about 65%, about 60%, about 55%, or about 50% cytosine nucleobases or derivatives or analogs thereof.

In some aspects, the C-rich RNA element comprises a sequence of about 3-20 nucleotides, about 4-18 nucleotides, about 6-16 nucleotides, about 6-14 nucleotides, about 6-12 nucleotides, about 6-10 nucleotides, or about 20, 19, 18, 17, 16, 15, 14, 13, 12, 11, 10, 9, 8, 7, 6, 5, 4, or 3 nucleotides. In some aspects, the C-rich RNA element comprises a sequence of about 6-12 nucleotides, wherein the sequence comprises 70-75%, 75-80%, 80-85%, 85-90% or 90-95% cytosine nucleobases, or derivatives or analogs thereof, optionally wherein the sequence is less than about 30-25%, 25-20%, 20-15%, 15-10%, or 10-5% adenosine and/or guanosine nucleobases, or derivatives or analogs thereof.

In any of the foregoing aspects, an mRNA comprises a C-rich RNA element comprising a sequence of linked nucleotides comprising the formula: 5′-[C1]ᵥ-[N1]_{w}-[N2]ₓ-[N3]_{y}-[C2]_{z}-3′, wherein C1 and C2 are nucleotides comprising cytidine, or a derivative or analogue thereof, wherein N1, and N2 and N3 if present, are each a nucleotide comprising a nucleobase selected from the group consisting of: adenine, guanine, thymine, uracil, and cytosine, and derivatives or analogues thereof, wherein v, w, x, y and z are integers whose value indicates the number of nucleotides comprising the C-rich RNA element, wherein v=2-15 nucleotides, wherein w=1-5 nucleotides, wherein x=0-5 nucleotides, wherein y=0-5 nucleotides, and wherein z=2-10 nucleotides. In some aspects, v=3-12 nucleotides, 5-10 nucleotides, 6-8 nucleotides, 3, 4, 5, 6, 7, 8, 9 or 10 nucleotides. In some aspects, z=2-7 nucleotides, 3-5 nucleotides, 2, 3, 4, 5, 6, or 7 nucleotides. In some aspects, w=1-3 nucleotides, 1, 2, or 3 nucleotide(s). In some aspects, x=0-3 nucleotides, 0, 1, 2, or 3 nucleotide(s). In some aspects, y=0-3 nucleotides, 0, 1, 2, or 3 nucleotide(s). In some aspects, N1 comprises adenosine, or derivative or analogue thereof; w=1 or 2; x=0, 1, 2, or 3; and y=0, 1, 2, or 3. In some aspects, N1 comprises adenosine, or derivative or analogue thereof; w=1 or 2; x=0; and y=0. In some aspects, N1 comprises uracil, or derivative or analogue thereof; w=1 or 2; N2 comprises adenosine, or derivative or analogue thereof; x=1, 2, or 3; N3 is guanosine, or derivative or analogue thereof; and y=1 or 2. In some aspects, N1 comprises uracil, or derivative or analogue thereof; w=1; N2 comprises adenosine, or derivative or analogue thereof; x=2; N3 is guanosine, or derivative or analogue thereof; and y=1.

In any of the foregoing aspects, an mRNA comprises a 5′UTR comprising a C-rich RNA element comprising the formula 5′-[C1]ᵥ-[N1]_{w}-[N2]ₓ-[N3]_{y}-[C2]_{z}-3′, wherein C1 and C2 are nucleotides comprising cytidine, or a derivative or analogue thereof, wherein N1, and N2 and N3 if present, are each a nucleotide comprising a nucleobase selected from the group consisting of: adenine, guanine, and uracil, and derivatives or analogues thereof, wherein v, w, x, y and z are integers whose value indicates the number of nucleotides comprising the C-rich RNA element, wherein v=4-10 nucleotides, wherein w=1-3 nucleotides, wherein x=0-3 nucleotides, wherein y=0-3 nucleotides, and wherein z=2-6 nucleotides. In some aspects, v=6-8 nucleotides, 6, 7, or 8 nucleotides. In some aspects, z=2-5 nucleotides, 2, 3, 4, or 5 nucleotides. In some aspects, w=1 or 2 nucleotide(s). In some aspects, x=0, 1 or 2 nucleotide(s). In some aspects, y=0 or 1 nucleotide(s). In some aspects, N1 comprises adenosine, or derivative or analogue thereof; w=1; x=0; and y=0. In some aspects, N1 comprises adenosine, or derivative or analogue thereof; w=2; x=0; and y=0. In some aspects, N1 comprises uracil, or derivative or analogue thereof; w=1 or 2; N2 comprises adenosine, or derivative or analogue thereof; x=1, 2, or 3; N3 is guanosine, or derivative or analogue thereof; and y=1 or 2. In some aspects, N1 comprises uracil, or derivative or analogue thereof; w=1; N2 comprises adenosine, or derivative or analogue thereof; x=2; N3 is guanosine, or derivative or analogue thereof; and y=1. In some aspects, wherein v=6-8; N1 comprises adenosine, or derivative or analogue thereof; w=1 or 2; x=0; y=0; and z=2-5. In some aspects, wherein v=6-8; N1 comprises uracil, or derivative or analogue thereof; w=1; N2 comprises adenosine, or derivative or analogue thereof; x=2; N3 is guanosine, or derivative or analogue thereof; y=1; and z=2-5.

In any of the foregoing aspects, the C-rich RNA element comprises the nucleotide sequence [5′-CCCCCCCCAACC-3′] set forth in SEQ ID NO 30 or comprises the nucleotide sequence [5′-CCCCCCCAACCC-3′] set forth in SEQ ID NO: 29.

In any of the foregoing aspects, the C-rich RNA element comprises the nucleotide sequence [5′-CCCCCCACCCCC-3′] set forth in SEQ ID NO: 31.

In any of the foregoing aspects, the C-rich RNA element comprises the nucleotide sequence [5′-CCCCCCUAAGCC-3′] set forth in SEQ ID NO: 32.

In any of the foregoing aspects, the C-rich RNA element comprises the nucleotide sequence [5′-CCCCACAACC-3′] set forth in SEQ ID NO: 33, or the nucleotide sequence [5′-CCCCCACAACC-3′] set forth in SEQ ID NO: 34.

In any of the foregoing aspects, the mRNA comprises a C-rich RNA element that is located about 40-50, about 30-40, about 20-30, about 10-20 or about 5-10 nucleotides downstream of the 5′ cap or 5′ end of the mRNA in the 5′ UTR. In some aspects, the C-rich RNA element is located about 15-20, about 10-15, about 5-10 nucleotides, about 1-5 nucleotides, or about 20, 19, 18, 17, 16, 15, 14, 13, 12, 11, 10, 9, 8, 7, 6, 5, 4, 3, 2 or 1 nucleotide downstream of the 5′ cap or 5′ end of the mRNA in the 5′ UTR. In some aspects, the C-rich RNA element is located about 5-10 nucleotides downstream of the 5′ cap or 5′end of the mRNA in the 5′ UTR.

In some aspects, the disclosure provides an mRNA, wherein the mRNA comprises: a 5′ cap, a 5′ UTR, an ORF encoding a polypeptide, and a 3′ UTR, wherein the 5′UTR comprises: (i) a structural RNA element comprising a stem loop comprising a nucleotide sequence at least 80%, 85%, 90%, 95%, 96%, 97%, 98%, 99% or 100% identical to the nucleotide sequence of SEQ ID NO: 6 or the nucleotide sequence of SEQ ID NO: 47; and (ii) a GC-rich RNA element comprising a nucleotide sequence selected from the group consisting of: SEQ ID NO: 1, SEQ ID NO: 2, SEQ ID NO: 3, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 49, SEQ ID NO: 50 and SEQ ID NO: 51.

In any of the foregoing aspects, the structural RNA element comprises a nucleotide sequence at least 80%, 85%, 90%, 95%, 96%, 97%, 98%, 99% or 100% identical to the nucleotide sequence of SEQ ID NO: 6. In some aspects, the structural RNA element has a deltaG (ΔG) of about −20 to −25 kcal/mol, about −15 to −20 kcal/mol, or about −10 to −15 kcal/mol. In some aspects, the structural RNA element comprises the nucleotide sequence of SEQ ID NO: 6.

In any of the foregoing aspects, the structural RNA element comprises a nucleotide sequence at least 80%, 85%, 90%, 95%, 96%, 97%, 98%, 99% or 100% identical to the nucleotide sequence of SEQ ID NO: 47. In some aspects, the structural RNA element has a deltaG (ΔG) of about −20 to −25 kcal/mol, about −15 to −20 kcal/mol, or about −10 to −15 kcal/mol. In some aspects, the structural RNA element comprises the nucleotide sequence of SEQ ID NO: 47.

In any of the foregoing aspects, the mRNA comprises: a 5′ cap, a 5′ UTR, an ORF encoding a polypeptide, and a 3′ UTR, wherein the 5′UTR comprises: (i) a structural RNA element comprising a stem loop comprising a nucleotide sequence at least 80%, 85%, 90%, 95%, 96%, 97%, 98%, 99% or 100% identical to the nucleotide sequence of SEQ ID NO: 6 or the nucleotide sequence of SEQ ID NO: 47; and (ii) a GC-rich RNA element comprising a nucleotide sequence selected from the group consisting of: SEQ ID NO: 1, SEQ ID NO: 2 and SEQ ID NO: 23. In some aspects, the GC-rich RNA element comprises the nucleotide sequence of SEQ ID NO: 1.

In any of the foregoing aspects, the mRNA comprises a Kozak-like sequence, and wherein the GC-rich RNA element is located about 1-20 nucleotides upstream of the Kozak-like sequence in the 5′ UTR. In some aspects, the GC-rich RNA element is located about 5, about 4, about 3, about 2, or 1 nucleotide upstream of the Kozak-like sequence in the 5′ UTR. In some aspects, the GC-rich RNA element is upstream of and immediately adjacent to the Kozak-like sequence in the 5′ UTR.

In any of the foregoing aspects, the mRNA comprises a structural RNA element that is upstream of the GC-rich RNA element in the 5′UTR. In some aspects, the structural RNA element is about 1-5, 5-10, 10-20, 20-30, 30-40, or 40-50 nucleotides, or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) upstream of the GC-rich RNA element in the 5′UTR. In some aspects, the structural RNA element is 1-5 nucleotides upstream of the GC-rich RNA element in the 5′UTR. In some aspects, the structural RNA element is 10-20 nucleotides upstream of the GC-rich RNA element in the 5′UTR. In some aspects, the structural RNA element is 30-40 nucleotides upstream of the GC-rich RNA element in the 5′UTR. In some aspects, the structural RNA element is upstream of and immediately adjacent to the GC-rich RNA element in the 5′UTR.

In any of the foregoing aspects, the mRNA comprises the Kozak-like sequence comprises the nucleotide sequence [5′-GCCACC-3′] set forth in SEQ ID NO: 17 or the nucleotide sequence [5′-GCCGCC-3′] set forth in SEQ ID NO: 48.

In any of the foregoing aspects, the structural RNA element is located downstream of the 5′ cap or 5′ end of the mRNA in the 5′ UTR. In some aspects, the structural RNA element is located about 45-50, about 40-45, about 35-40, about 30-35, about 25-30, about 20-25, about 15-20, about 10-15, about 5-10 nucleotides, about 1-5 nucleotide(s), or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) downstream of the 5′ cap or 5′ end of the mRNA in the 5′ UTR. In some aspects, the structural RNA element is located about 40-45 nucleotides downstream of the 5′ cap or 5′ end of the mRNA in the 5′ UTR. In some aspects, the structural RNA element is located about 20-25 nucleotides downstream of the 5′ cap or 5′ end of the mRNA in the 5′ UTR. In some aspects, the structural RNA element is located about 5-10 nucleotides downstream of the 5′ cap or 5′ end of the mRNA in the 5′ UTR. In some aspects, the structural RNA element is located downstream of the 5′ cap or 5′ end of the mRNA and immediately adjacent to a transcription start site element in the 5′ UTR. In some aspects, the transcription start site element comprises the nucleotide sequence [5′-GGGAAA-3′] set forth in SEQ ID NO: 53 or the nucleotide sequence [5′-AGGAAA-3′] set forth in SEQ ID NO: 54.

In any of the foregoing aspects, the mRNA comprises a 5′UTR wherein the 5′UTR comprises a C-rich RNA element comprising a nucleotide sequence selected from the group consisting of: SEQ ID NO: 29, SEQ ID NO: 30, SEQ ID NO: 31, SEQ ID NO: 32, SEQ ID NO: 33 and SEQ ID NO: 34. In some aspects, the C-rich RNA element is proximal to the 5′ cap or 5′ end of the mRNA and upstream of each of the structural RNA element and the GC-rich RNA element in the 5′UTR. In some aspects, the C-rich RNA element is about 1-5, 5-10, 10-20, 20-30, 30-40, or 40-50 nucleotides, or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) upstream of the structural RNA element in the 5′UTR. In some aspects, the C-rich RNA element is 20-30 nucleotides upstream of the structural RNA element in the 5′UTR. In some aspects, the C-rich RNA element is 30-40 nucleotides upstream of the structural RNA element in the 5′UTR. In some aspects, the C-rich RNA element is 40-50 nucleotides upstream of the structural RNA element in the 5′UTR. In some aspects, the C-rich RNA element is located downstream of the 5′ cap or 5′ end of the mRNA and upstream of each of the structural RNA element and the GC-rich RNA element in the 5′UTR. In some aspects, the C-rich RNA element is located about 20-25, about 15-20, about 10-15, about 5-10 nucleotides, about 1-10, about 1-8, about 1-6, or about 1-3 nucleotide(s), or about 20, 19, 18, 17, 16, 15, 14, 13, 12, 11, 10, 9, 8, 7, 6, 5, 4, 3, 2 or 1 nucleotide(s) downstream of the 5′ cap or 5′ end of the mRNA in the 5′ UTR. In some aspects, the C-rich RNA element is located about 1-10 nucleotides downstream of the 5′ cap or 5′ end of the mRNA in the 5′ UTR. In some aspects, the C-rich RNA element is located about 5-10 nucleotides downstream of the 5′ cap or 5′ end of the mRNA in the 5′ UTR. In some aspects, the C-rich RNA element is located about 1-6 nucleotides downstream of the 5′ cap or 5′ end of the mRNA in the 5′ UTR.

In any of the foregoing aspects, the mRNA comprises a 5′UTR wherein the 5′UTR comprises a C-rich RNA element, wherein the C-rich RNA element is downstream of immediately adjacent to a transcription start site element and upstream of each of the structural RNA element and the GC-rich RNA element in the 5′UTR. In some aspects, the transcription start site element comprises the nucleotide sequence [5′-GGGAAA-3′] set forth in SEQ ID NO: 53 or the nucleotide sequence [5′-AGGAAA-3′] set forth in SEQ ID NO: 54.

In some aspects, the disclosure provides an mRNA, wherein the mRNA comprising a 5′ cap, a 5′ UTR comprising a Kozak-like sequence upstream of an initiation codon, an ORF encoding a polypeptide, and a 3′ UTR, wherein the 5′ UTR comprises from 5′ to 3′: (i) a C-rich RNA element located proximal to the 5′ cap, wherein the C-rich RNA element comprises a nucleotide sequence selected from selected from the group consisting of SEQ ID NO: 31, SEQ ID NO: 32 and SEQ ID NO: 33; (ii) a structural RNA element comprising a stem loop located downstream of the C-rich RNA element, wherein the structural RNA element comprises the nucleotide sequence of SEQ ID NO: 6 or the nucleotide sequence of SEQ ID NO: 47; and (iii) a GC-rich RNA element located downstream of the structural RNA element and proximal to the Kozak-like sequence, wherein the GC-rich RNA element comprises a nucleotide sequence selected from the group consisting of: SEQ ID NO: 1, SEQ ID NO: 2 and SEQ ID NO: 23.

In any of the foregoing aspects, the mRNA comprises a 5′ cap, a 5′ UTR comprising a Kozak-like sequence upstream of an initiation codon, an ORF encoding a polypeptide, and a 3′ UTR, wherein (i) the C-rich RNA element comprises the nucleotide sequence set forth in SEQ ID NO: 31; (ii) the structural RNA element comprises the nucleotide sequence of SEQ ID NO: 6; and (iii) the GC-rich RNA element comprises the nucleotide sequence set forth in SEQ ID NO: 1.

In any of the foregoing aspects, the mRNA comprises a 5′ cap, a 5′ UTR comprising a Kozak-like sequence upstream of an initiation codon, an ORF encoding a polypeptide, and a 3′ UTR, wherein (i) the C-rich RNA element comprises the nucleotide sequence set forth in SEQ ID NO: 33; (ii) the structural RNA element comprises the nucleotide sequence of SEQ ID NO: 6; and (iii) the GC-rich RNA element comprises the nucleotide sequence set forth in SEQ ID NO: 1.

In any of the foregoing aspects, the mRNA comprises a 5′ cap, a 5′ UTR comprising a Kozak-like sequence upstream of an initiation codon, an ORF encoding a polypeptide, and a 3′ UTR, wherein (i) the C-rich RNA element comprises the nucleotide sequence set forth in SEQ ID NO: 32; (ii) the structural RNA element comprises the nucleotide sequence of SEQ ID NO: 6; and (iii) the GC-rich RNA element comprises the nucleotide sequence set forth in SEQ ID NO: 23.

In any of the foregoing aspects, the C-rich RNA element is located about 10-15, about 5-10 nucleotides, about 1-10, about 1-8, about 1-6, or about 1-3 nucleotide(s), or about 15, 14, 13, 12, 11, 10, 9, 8, 7, 6, 5, 4, 3, 2 or 1 nucleotide(s) downstream of the 5′ cap or 5′ end of the mRNA in the 5′ UTR. In some aspects, the C-rich RNA element is located about 1-10 nucleotides downstream of the 5′ cap or 5′ end of the mRNA in the 5′ UTR. In some aspects, the C-rich RNA element is located about 5-10 nucleotides downstream of the 5′ cap or 5′ end of the mRNA in the 5′ UTR. In some aspects, the C-rich RNA element is located about 1-6 nucleotides downstream of the 5′ cap or 5′ end of the mRNA in the 5′ UTR. In some aspects, the C-rich RNA element is downstream of immediately adjacent to a transcription start site element, wherein the transcription start site element comprises the nucleotide sequence [5′-GGGAAA-3′] set forth in SEQ ID NO: 53 or the nucleotide sequence [5′-AGGAAA-3′] set forth in SEQ ID NO: 54.

In any of the foregoing aspects, the mRNA comprises a structural RNA element, wherein the structural RNA element is located about 45-50, about 40-45, about 35-40, about 30-35, about 25-30, about 20-25, about 15-20, about 10-15, about 5-10 nucleotides, about 1-5 nucleotide(s), or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) downstream of the C-rich RNA element in the 5′ UTR. In some aspects, the structural RNA element is located about 40-45 nucleotides downstream of the C-rich RNA element in the 5′ UTR. In some aspects, the structural RNA element is located about 35-40 nucleotides downstream of the C-rich RNA element in the 5′ UTR. In some aspects, the structural RNA element is located about 30-35 nucleotides downstream of the C-rich RNA element in the 5′ UTR.

In any of the foregoing aspects, the mRNA comprises a GC-rich RNA element, wherein the GC-rich RNA element is located about 10-15, about 5-10, or about 1-5 nucleotides downstream of the structural RNA element in the 5′ UTR. In some aspects, the GC-rich RNA element is located about 5, about 4, about 3, about 2, or 1 nucleotide downstream of the structural RNA element in the 5′ UTR. In some aspects, the GC-rich RNA element is upstream of and immediately adjacent to the Kozak-like sequence in the 5′ UTR.

In any of the foregoing aspects, an mRNA comprises a 5′UTR, wherein the 5′ UTR comprises the nucleotide sequence of SEQ ID NO: 4, wherein a structural RNA element comprising a stem-loop is inserted, optionally wherein a GC-rich RNA element is inserted, optionally wherein a C-rich RNA element is inserted.

In some aspects, the disclosure provides an mRNA, wherein the mRNA comprises: a 5′ cap, a 5′ UTR comprising a structural RNA element comprising a stem-loop, an ORF encoding a polypeptide, and a 3′ UTR, wherein the structural RNA element comprises a nucleotide sequence at least 80%, 85%, 90%, 95%, 96%, 97%, 98%, 99% or 100% identical to the nucleotide sequence of SEQ ID NO: 6, wherein the 5′ UTR comprises the nucleotide sequence of SEQ ID NO: 4 or SEQ ID NO: 60 comprising a GC-rich RNA element comprising the sequence CCCCGGCGCC (SEQ ID NO: 1), and wherein the structural RNA element is inserted upstream of the GC-rich RNA element in the 5′ UTR.

In some aspects, the disclosure provides an mRNA, wherein the mRNA comprises: a 5′ cap, a 5′ UTR comprising a structural RNA element comprising a stem-loop, an ORF encoding a polypeptide, and a 3′ UTR, wherein the structural RNA element comprises a sequence of 15-25 linked nucleotides comprising at least 60% G/C bases, wherein the structural RNA element comprises (i) a double-stranded stem of about 4-7 base pairs; (ii) a single-stranded loop of about 4-7 nucleotides; (iii) a nucleotide sequence which differs from SEQ ID NO: 6 by substitution, deletion or insertion of 1, 2, 3, 4, or 5 nucleotides; and (iv) a delta G (ΔG) of about −10 to −15 kcal/mol, wherein the 5′ UTR comprises the nucleotide sequence of SEQ ID NO: 4 or SEQ ID NO: 60 comprising a GC-rich RNA element comprising the sequence CCCCGGCGCC (SEQ ID NO: 1), and wherein the structural RNA element is inserted upstream of the GC-rich RNA element in the 5′ UTR.

In some aspects, the disclosure provides an mRNA, wherein the mRNA comprises: a 5′ cap, a 5′ UTR comprising a structural RNA element comprising a stem-loop, an ORF encoding a polypeptide, and a 3′ UTR, wherein the structural RNA element comprises the nucleotide sequence of SEQ ID NO: 6, wherein the 5′ UTR comprises the nucleotide sequence of SEQ ID NO: 4 or SEQ ID NO: 60 comprising a GC-rich RNA element comprising the sequence CCCCGGCGCC (SEQ ID NO: 1), and wherein the structural RNA element is inserted upstream of the GC-rich RNA element in the 5′ UTR.

In any of the foregoing aspects, the mRNA comprises a structural RNA element, wherein the structural RNA element is inserted about 1-5, 5-10, 10-20, 20-30, or 30-40 nucleotides, or about 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) upstream of the GC-rich RNA element in SEQ ID NO: 4 or SEQ ID NO: 60. In some aspects, the structural RNA element is inserted 1-5 nucleotides upstream of the GC-rich RNA element in SEQ ID NO: 4 or SEQ ID NO: 60. In some aspects, the structural RNA element is inserted 10-20 nucleotides upstream of the GC-rich RNA element in SEQ ID NO: 4 or SEQ ID NO: 60. In some aspects, the structural RNA element is inserted 30-40 nucleotides upstream of the GC-rich RNA element in SEQ ID NO: 4 or SEQ ID NO: 60. In some aspects, the structural RNA element is inserted upstream of and immediately adjacent to the GC-rich RNA element in SEQ ID NO: 4 or SEQ ID NO: 60.

In any of the foregoing aspects, the mRNA comprises a C-rich RNA element inserted proximal to the 5′ cap of the mRNA in SEQ ID NO: 4 or SEQ ID NO: 60, wherein the C-rich RNA element comprises a nucleotide sequence selected from selected from the group consisting of SEQ ID NO: 31, SEQ ID NO: 32 and SEQ ID NO: 33. In some aspects, the C-rich RNA element comprises the nucleotide sequence of SEQ ID NO: 31. In some aspects, the C-rich RNA element is inserted about 1-10 nucleotides downstream of the 5′ cap in SEQ ID NO: 4 or SEQ ID NO: 60. In some aspects, the C-rich RNA element is inserted about 5-10 nucleotides downstream of the 5′ cap in SEQ ID NO: 4 or SEQ ID NO: 60. In some aspects, the C-rich RNA element is inserted about 1-6 nucleotides downstream of the 5′ cap of in SEQ ID NO: 4 or SEQ ID NO: 60. In some aspects, the C-rich RNA element is downstream of and immediately adjacent to a transcription start site element in the 5′UTR, wherein the transcription start site element comprises the nucleotide sequence [5′-GGGAAA-3′] in SEQ ID NO: 4 or the nucleotide sequence [5′-AGGAAA-3′] in SEQ ID NO: 60.

In some aspects, the disclosure provides an mRNA, wherein the mRNA comprises: a 5′ cap, a 5′ UTR, an ORF encoding a polypeptide, and a 3′ UTR, wherein the 5′ UTR comprises a nucleotide sequence selected from the group consisting of: (i) the nucleotide sequence of SEQ ID NO: 116, (ii)
the nucleotide sequence of SEQ ID NO: 120, (iii) the nucleotide sequence of SEQ ID NO: 124, (iv) the nucleotide sequence of SEQ ID NO: 128, and (v) the nucleotide sequence of SEQ ID NO: 41.

In any of the foregoing aspects, an mRNA comprises: a 5′ cap, a 5′ UTR, an ORF encoding a polypeptide, and a 3′ UTR, wherein the 3′UTR comprises a nucleotide sequence of a 3′UTR of a nuclear-encoded mitochondrially derived protein (NEMP). In some aspects, binding of the 3′UTR to one or more RNA-binding proteins promotes the stabilization, localization, and/or translation of the mRNA. In some aspects, the NEMP is selected from the group consisting of: human OXAL1, human MRPS12, and mouse Sod2. In some aspects, the nucleotide sequence of the 3′UTR is at least 70%, at least 75%, at least 80%, at least 85%, at least 90%, at least 95%, at least 96%, at least 97%, at least 98%, at least 99%, or 100% identical to the nucleotide sequence of the NEMP 3′UTR. In some aspects, the 3′UTR differs from the nucleotide sequence of the NEMP 3′UTR by 1-5, 5-10, 10-15, 15-20, 20-25, 25-30, 30-35, 35-40, 40-45, 45-50 or about 50 or more nucleotides.

In any of the foregoing aspects, an mRNA comprises: a 5′ cap, a 5′ UTR, an ORF encoding a polypeptide, and a 3′UTR of a nuclear-encoded mitochondrially derived protein (NEMP), wherein the 3′UTR is about 50-100 nucleotides, about 100-200 nucleotides, about 200-300 nucleotides, about 300-400 nucleotides, about 400-500 nucleotides, about 500-600, about 600-700 nucleotides, about 700-800 nucleotides, about 800-900 nucleotides, about 900-1000 nucleotides, about 1000-1100 nucleotides, about 1100-1200 nucleotides, about 1200-1300 nucleotides, about 1300-1400 nucleotides, or about 1400-1500 nucleotides in length.

In any of the foregoing aspects, the 3′ UTR comprises a nucleotide sequence at least 70%, at least 75%, at least 80%, at least 85%, at least 90%, at least 95%, at least 96%, at least 97%, at least 98%, at least 99% identical or 100% identical to a nucleotide sequence selected from the group consisting of: SEQ ID NO: 72, SEQ ID NO: 74; SEQ ID NO: 76; and SEQ ID NO: 78. In some aspects, the 3′UTR comprises a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 72. In some aspects, the 3′UTR comprises a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 74. In some aspects, the 3′UTR comprises a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 76. In some aspects, the 3′UTR comprises a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 78.

In any of the foregoing aspects, the 3′UTR differs from the NEMP 3′UTR by about 1-5, 5-10, 10-15, 15-20, 20-25, 25-30, 30-35, 35-40, 40-45, 45-50 or about 50-100 nucleotides, wherein the NEMP 3′UTR comprises a nucleotide sequence selected from the group consisting of: SEQ ID NO: 72, SEQ ID NO: 74; SEQ ID NO: 76; and SEQ ID NO: 78. In some aspects, the 3′UTR comprises a nucleotide sequence selected from the group consisting of: SEQ ID NO: 72, SEQ ID NO: 74; SEQ ID NO: 76; and SEQ ID NO: 78. In some aspects, the 3′UTR comprises the nucleotide sequence of SEQ ID NO: 72. In some aspects, the 3′UTR comprises the nucleotide sequence of SEQ ID NO: 74. In some aspects, the 3′UTR comprises the nucleotide sequence of SEQ ID NO: 76. In some aspects, the 3′UTR comprises the nucleotide sequence of SEQ ID NO: 78.

In any of the foregoing aspects, the 3′ UTR comprises one or more microRNA (miRNA) binding sites. In some aspects, the 3′UTR comprises 1, 2, 3, 4, 5, 6, 7, 8, 9, or 10 miRNA binding site(s). In some aspects, the 3′UTR comprises 1, 2, 3 or 4 miRNA binding sites. In some aspects, the miRNA binding site is targeted by miR-142-3p or miR-142-5p. In some aspects, the miRNA binding site comprises a nucleotide sequence at least 80%, at least 85%, at least 90%, at least 95%, at least 96%, at least 97%, at least 98%, at least 99%, or 100% identical to the nucleotide sequence of SEQ ID NO: 179 or SEQ ID NO: 181. In some aspects, the miRNA binding site comprises the nucleotide sequence of SEQ ID NO: 179. In some aspects, the miRNA binding site comprises the nucleotide sequence of SEQ ID NO: 181.

In any of the foregoing aspects, an mRNA comprises a 3′UTR, wherein the 3′UTR comprises one or more stop codons at the 5′end of the 3′UTR, and wherein the 3′UTR comprises 1, 2, 3, or 4 miRNA binding sites located proximal to the one or more stop codons. In some aspects, the miRNA binding site(s) are located downstream of and immediately adjacent to the one or more stop codons at the 5′end of the 3′UTR. In some aspects, the miRNA binding sites are located about 45-50, about 40-45, about 35-40, about 30-35, about 25-30, about 20-25, about 15-20, about 10-15, about 6-10 nucleotides, about 1-5 nucleotide(s), or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) downstream of the one or more stop codons at the 5′end of the 3′UTR.

In some aspects, the miRNA binding sites are located about 10, about 9, about 8, about 7, about 6, about 5, about 4, about 3, about 2, or about 1 nucleotide(s) downstream of the one or more stop codons at the 5′end of the 3′UTR.

In any of the foregoing aspects, an mRNA comprises a 3′UTR, wherein the 3′UTR comprises 1, 2, 3, or 4 miRNA binding sites located proximal to the 3′end of the 3′UTR. In some aspects, the miRNA binding site(s) are located upstream of and immediately adjacent to the 3′end of the 3′UTR. In some aspects, the miRNA binding site(s) are located about 1-5, about 6-10, about 10-15, about 15-20, about 20-25, about 25-30, about 30-35, about 35-40, about 40-45, or about 45-50 nucleotide(s) or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotide(s) upstream of the 3′end of the 3′UTR. In some aspects, the miRNA binding site(s) are located about 1, about 2, about 3, about 4, or about 5, about 6, about 7, about 8, about 9 or about 10 nucleotide(s) upstream of the 3′end of the 3′UTR.

In any of the foregoing aspects, an mRNA comprises a 3′UTR, wherein the 3′UTR comprises 1, 2, 3, 4, 5, 6, 7, 8, 9, or 10 miRNA binding sites, wherein an upstream miRNA binding site is located directly adjacent to one or more downstream miRNA binding site(s). In some aspects, an upstream miRNA binding site is separated from a downstream miRNA binding site by about 1-5, about 1-10, about 5-10, about 5-15, about 10-20, about 15-20, about 15-30, or about 20-30 nucleotide(s) or about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotide(s). In some aspects, an upstream miRNA binding site is separated from a downstream miRNA binding site by about 1, about 2, about 3, about 4, about 5, about 6, about 7, about 8, about 9 or about 10 nucleotide(s).

In any of the foregoing aspects, an mRNA comprises a 3′UTR, wherein the 3′ UTR comprises a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 78, wherein the 3′UTR comprises 1, 2, 3, or 4 miR-142-3p binding sites, and wherein the miR-142-3p binding site comprises the nucleotide sequence of SEQ ID NO: 179. In some aspects, the 1, 2, 3, or 4 miR-142-3p binding sites are located proximal to the 3′end or the 3′UTR. In some aspects, the 3′UTR comprises the nucleotide sequence of SEQ ID NO: 170.

In any of the foregoing aspects, an mRNA comprises a 3′UTR, wherein the 3′UTR comprises one or more stop codons at the 5′end and wherein the 1, 2, 3, or 4 miR-142-3p binding sites are located proximal to the one or more stop codons. In some aspects, the 3′UTR comprises the nucleotide sequence of SEQ ID NO: 172.

In any of the foregoing aspects, an mRNA comprises: a 5′ cap, a 5′ UTR, an ORF encoding a polypeptide, and a 3′UTR, wherein the 3′UTR comprises a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 76, wherein the 3′UTR comprises 1, 2, 3, or 4 miR-142-3p binding sites, and wherein the miR-142-3p binding site comprises the nucleotide sequence of SEQ ID NO: 179. In some aspects, the 1, 2, 3, or 4 miR-142-3p binding sites are located proximal to the 3′end or the 3′UTR. In some aspects, the 3′UTR comprises the nucleotide sequence of SEQ ID NO: 174. In some aspects, the 3′UTR comprises one or more stop codons at the 5′end and wherein the 1, 2, 3, or 4 miR-142-3p binding sites are located proximal to the one or more stop codons. In some aspects, the 3′UTR comprises the nucleotide sequence of SEQ ID NO: 176.

In some aspects, the disclosure provides an mRNA, wherein the mRNA comprises: a 5′ cap, a 5′UTR, an ORF encoding a polypeptide, and a 3′UTR, wherein the 3′UTR comprises a nucleotide sequence of a 3′UTR of a NEMP. In some aspects, binding of the 3′UTR to one or more RNA-binding proteins promotes the stabilization, localization, and/or translation of the mRNA. In some aspects, the NEMP is selected from the group consisting of: human OXAL1, human MRPS12, and mouse Sod2. In some aspects, the nucleotide sequence of the 3′UTR is at least 70%, at least 75%, at least 80%, at least 85%, at least 90%, at least 95%, at least 96%, at least 97%, at least 98%, at least 99%, or 100% identical to the nucleotide sequence of the NEMP 3′UTR. In some aspects, the 3′UTR differs from the nucleotide sequence of the NEMP 3′UTR by 1-5, 5-10, 10-15, 15-20, 20-25, 25-30, 30-35, 35-40, 40-45, 45-50 or about 50 or more nucleotides. In some aspects, the 3′UTR is about 50-100 nucleotides, about 100-200 nucleotides, about 200-300 nucleotides, about 300-400 nucleotides, about 400-500 nucleotides, about 500-600, about 600-700 nucleotides, about 700-800 nucleotides, about 800-900 nucleotides, about 900-1000 nucleotides, about 1000-1100 nucleotides, about 1100-1200 nucleotides, about 1200-1300 nucleotides, about 1300-1400 nucleotides, or about 1400-1500 nucleotides in length.

In any of the foregoing aspects, the mRNA comprises: a 5′ cap, a 5′UTR, an ORF encoding a polypeptide, and a 3′UTR, wherein the 3′UTR comprises a nucleotide sequence at least 70%, at least 75%, at least 80%, at least 85%, at least 90%, at least 95%, at least 96%, at least 97%, at least 98%, at least 99% identical or 100% identical to a nucleotide sequence selected from the group consisting of: SEQ ID NO: 72; SEQ ID NO: 74; SEQ ID NO: 76; and SEQ ID NO: 78. In some aspects, the 3′UTR comprises a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 72. In some aspects, the 3′UTR comprises a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 74. In some aspects, the 3′UTR comprises a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 76. In some aspects, the 3′UTR comprises a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 78.

In any of the foregoing aspects, the mRNA comprises: a 5′ cap, a 5′UTR, an ORF encoding a polypeptide, and a 3′UTR, wherein the 3′UTR differs from the NEMP 3′UTR by about 1-5, 5-10, 10-15, 15-20, 20-25, 25-30, 30-35, 35-40, 40-45, 45-50 or about 50-100 nucleotides, wherein the NEMP 3′UTR comprises a nucleotide sequence selected from the group consisting of: SEQ ID NO: 72; SEQ ID NO: 74; SEQ ID NO: 76; and SEQ ID NO: 78.

In any of the foregoing aspects, the mRNA comprises: a 5′ cap, a 5′UTR, an ORF encoding a polypeptide, and a 3′UTR, wherein the 3′UTR comprises a nucleotide sequence selected from the group consisting of: SEQ ID NO: 72; SEQ ID NO: 74; SEQ ID NO: 76; and SEQ ID NO: 78. In some aspects, the 3′UTR comprises the nucleotide sequence of SEQ ID NO: 72. In some aspects, the 3′UTR comprises the nucleotide sequence of SEQ ID NO: 74. In some aspects, the 3′UTR comprises the nucleotide sequence of SEQ ID NO: 76. In some aspects, the 3′UTR comprises the nucleotide sequence of SEQ ID NO: 78.

In any of the foregoing aspects, the disclosure provides an mRNA, wherein the mRNA comprises: a 5′ cap, a 5′UTR, an ORF encoding a polypeptide, and a 3′UTR comprising a nucleotide sequence of a 3′UTR of a NEMP, wherein the 3′UTR comprises one or more microRNA (miRNA) binding sites. In some aspects, the 3′UTR comprises 1, 2, 3, 4, 5, 6, 7, 8, 9, or 10 miRNA binding site(s).

In some aspects, the 3′UTR comprises 1, 2, 3 or 4 miRNA binding sites. In some aspects, the miRNA binding site is targeted by miR-142-3p or miR-142-5p. In some aspects, the miRNA binding site comprises a nucleotide sequence at least 80%, at least 85%, at least 90%, at least 95%, at least 96%, at least 97%, at least 98%, at least 99%, or 100% identical to the nucleotide sequence of SEQ ID NO: 179 or SEQ ID NO: 181. In some aspects, the miRNA binding site comprises the nucleotide sequence of SEQ ID NO: 179. In some aspects, the miRNA binding site comprises the nucleotide sequence of SEQ ID NO: 181.

In any of the foregoing aspects, the disclosure provides an mRNA, wherein the mRNA comprises: a 5′ cap, a 5′UTR, an ORF encoding a polypeptide, and a 3′UTR comprising a nucleotide sequence of a 3′UTR of a NEMP, wherein the 3′UTR comprises one or more stop codons at the 5′end of the 3′UTR, and wherein the 3′UTR comprises 1, 2, 3, or 4 miRNA binding sites located proximal to the one or more stop codons. In some aspects, the miRNA binding site(s) are located downstream of and immediately adjacent to the one or more stop codons at the 5′end of the 3′UTR. In some aspects, the miRNA binding sites are located about 45-50, about 40-45, about 35-40, about 30-35, about 25-30, about 20-25, about 15-20, about 10-15, about 6-10 nucleotides, about 1-5 nucleotide(s), or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) downstream of the one or more stop codons at the 5′end of the 3′UTR. In some aspects, the miRNA binding sites are located about 10, about 9, about 8, about 7, about 6, about 5, about 4, about 3, about 2, or about 1 nucleotide(s) downstream of the one or more stop codons at the 5′end of the 3′UTR.

In any of the foregoing aspects, the disclosure provides an mRNA, wherein the mRNA comprises: a 5′ cap, a 5′UTR, an ORF encoding a polypeptide, and a 3′UTR comprising a nucleotide sequence of a 3′UTR of a NEMP, wherein the 3′UTR comprises 1, 2, 3, or 4 miRNA binding sites located proximal to the 3′end of the 3′UTR. In some aspects, the miRNA binding site(s) are located upstream of and immediately adjacent to the 3′end of the 3′UTR. In some aspects, the miRNA binding site(s) are located about 1-5, about 6-10, about 10-15, about 15-20, about 20-25, about 25-30, about 30-35, about 35-40, about 40-45, or about 45-50 nucleotide(s) or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotide(s) upstream of the 3′end of the 3′UTR. In some aspects, the miRNA binding site(s) are located about 1, about 2, about 3, about 4, or about 5, about 6, about 7, about 8, about 9 or about 10 nucleotide(s) upstream of the 3′end of the 3′UTR.

In any of the foregoing aspects, the disclosure provides an mRNA, wherein the mRNA comprises: a 5′ cap, a 5′UTR, an ORF encoding a polypeptide, and a 3′UTR comprising a nucleotide sequence of a 3′UTR of a NEMP, wherein the 3′UTR comprises 1, 2, 3, 4, 5, 6, 7, 8, 9, or 10 miRNA binding sites, wherein an upstream miRNA binding site is located directly adjacent to one or more downstream miRNA binding site(s).

In any of the foregoing aspects, the disclosure provides an mRNA, wherein the mRNA comprises: a 5′ cap, a 5′UTR, an ORF encoding a polypeptide, and a 3′UTR comprising a nucleotide sequence of a 3′UTR of a NEMP, wherein the 3′UTR comprises 1, 2, 3, 4, 5, 6, 7, 8, 9, or 10 miRNA binding sites, wherein an upstream miRNA binding site is separated from a downstream miRNA binding site by about 1-5, about 1-10, about 5-10, about 5-15, about 10-20, about 15-20, about 15-30, or about 20-30 nucleotide(s) or about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotide(s).

In any of the foregoing aspects, the disclosure provides an mRNA, wherein the mRNA comprises: a 5′ cap, a 5′UTR, an ORF encoding a polypeptide, and a 3′UTR comprising a nucleotide sequence of a 3′UTR of a NEMP, wherein the 3′UTR comprises 1, 2, 3, 4, 5, 6, 7, 8, 9, or 10 miRNA binding sites, wherein an upstream miRNA binding site is separated from a downstream miRNA binding site by about 1, about 2, about 3, about 4, about 5, about 6, about 7, about 8, about 9 or about 10 nucleotide(s).

In any of the foregoing aspects, the disclosure provides an mRNA, wherein the mRNA comprises: a 5′ cap, a 5′UTR, an ORF encoding a polypeptide, and a 3′UTR comprising a nucleotide sequence of a 3′UTR of a NEMP, wherein the 3′ UTR comprises a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 78, wherein the 3′UTR comprises 1, 2, 3, or 4 miR-142-3p binding sites, and wherein the miR-142-3p binding site comprises the nucleotide sequence of SEQ ID NO: 179. In some aspects, the 1, 2, 3, or 4 miR-142-3p binding sites are located proximal to the 3′end or the 3′UTR. In some aspects, the 3′UTR comprises the nucleotide sequence of SEQ ID NO: 170. In some aspects, the 3′UTR comprises one or more stop codons at the 5′end and wherein the 1, 2, 3, or 4 miR-142-3p binding sites are located proximal to the one or more stop codons. In some aspects, the 3′UTR comprises the nucleotide sequence of SEQ ID NO: 172.

In any of the foregoing aspects, the disclosure provides an mRNA, wherein the mRNA comprises: a 5′ cap, a 5′UTR, an ORF encoding a polypeptide, and a 3′UTR comprising a nucleotide sequence of a 3′UTR of a NEMP, wherein the 3′UTR comprises a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 76, wherein the 3′UTR comprises 1, 2, 3, or 4 miR-142-3p binding sites, and wherein the miR-142-3p binding site comprises the nucleotide sequence of SEQ ID NO: 179. In some aspects, the 1, 2, 3, or 4 miR-142-3p binding sites are located proximal to the 3′end or the 3′UTR. In some aspects, the 3′UTR comprises the nucleotide sequence of SEQ ID NO: 174. In some aspects, the 3′UTR comprises one or more stop codons at the 5′end and wherein the 1, 2, 3, or 4 miR-142-3p binding sites are located proximal to the one or more stop codons. In some aspects, the 3′UTR comprises the nucleotide sequence of SEQ ID NO: 176.

In some aspects, the disclosure provides an mRNA, wherein the mRNA comprises: a 5′ cap, a 5′ UTR comprising a structural RNA element comprising a stem-loop, wherein the structural RNA element comprises a nucleotide sequence at least 80%, 85%, 90%, 95%, 96%, 97%, 98%, 99% or 100% identical to the nucleotide sequence of SEQ ID NO: 6, an ORF encoding a polypeptide, and a 3′ UTR, wherein the 3′UTR comprises a nucleotide sequence at least 70%, at least 75%, at least 80%, at least 85%, at least 90%, at least 95%, at least 96%, at least 97%, at least 98%, at least 99% identical or 100% identical to a nucleotide sequence of SEQ ID NO: 76 or SEQ ID NO: 78, wherein the 5′ UTR comprises the nucleotide sequence of SEQ ID NO: 4 or SEQ ID NO: 60 comprising a GC-rich RNA element comprising the sequence CCCCGGCGCC (SEQ ID NO: 1), and wherein the structural RNA element is inserted upstream of the GC-rich RNA element in the 5′ UTR. In some aspects, the structural RNA element is inserted about 1-5, 5-10, 10-20, 20-30, or 30-40 nucleotides, or about 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) upstream of the GC-rich RNA element in SEQ ID NO: 4 or SEQ ID NO: 60.

In some aspects, the disclosure provides an mRNA, wherein the mRNA comprises: a 5′ cap, a 5′ UTR comprising a structural RNA element comprising a stem-loop wherein the structural RNA element comprises a sequence of 15-25 linked nucleotides comprising at least 60% G/C bases, wherein the structural RNA element comprises (i) a double-stranded stem of about 4-7 base pairs; (ii) a single-stranded loop of about 4-7 nucleotides; (iii) a nucleotide sequence which differs from SEQ ID NO: 6 by substitution, deletion or insertion of 1, 2, 3, 4, or 5 nucleotides; and (iv) a delta G (ΔG) of about −10 to −15 kcal/mol, an ORF encoding a polypeptide, and a 3′ UTR, wherein the 3′UTR comprises a nucleotide sequence at least 70%, at least 75%, at least 80%, at least 85%, at least 90%, at least 95%, at least 96%, at least 97%, at least 98%, at least 99% identical or 100% identical to a nucleotide sequence of SEQ ID NO: 76 or SEQ ID NO: 78, wherein the 5′ UTR comprises the nucleotide sequence of SEQ ID NO: 4 or SEQ ID NO: 60 comprising a GC-rich RNA element comprising the sequence CCCCGGCGCC (SEQ ID NO: 1), and wherein the structural RNA element is inserted upstream of the GC-rich RNA element in the 5′ UTR. In some aspects, the structural RNA element is inserted about 1-5, 5-10, 10-20, 20-30, or 30-40 nucleotides, or about 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) upstream of the GC-rich RNA element in SEQ ID NO: 4 or SEQ ID NO: 60.

In some aspects, the disclosure provides an mRNA, wherein the mRNA comprises: a 5′ cap, a 5′ UTR comprising a structural RNA element comprising the nucleotide sequence of SEQ ID NO: 6, an ORF encoding a polypeptide, and a 3′ UTR, wherein the 3′UTR comprises a nucleotide sequence at least 70%, at least 75%, at least 80%, at least 85%, at least 90%, at least 95%, at least 96%, at least 97%, at least 98%, at least 99% identical or 100% identical to a nucleotide sequence of SEQ ID NO: 76 or SEQ ID NO: 78, wherein the 5′ UTR comprises the nucleotide sequence of SEQ ID NO: 4 or SEQ ID NO: 60 comprising a GC-rich RNA element comprising the sequence CCCCGGCGCC (SEQ ID NO: 1), and wherein the structural RNA element is inserted upstream of the GC-rich RNA element in the 5′ UTR. In some aspects, the structural RNA element is inserted about 1-5, 5-10, 10-20, 20-30, or 30-40 nucleotides, or about 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) upstream of the GC-rich RNA element in SEQ ID NO: 4 or SEQ ID NO: 60. In some aspects, the structural RNA element is inserted 1-5 nucleotides upstream of the GC-rich RNA element in SEQ ID NO: 4 or SEQ ID NO: 60. In some aspects, the structural RNA element is inserted 10-20 nucleotides upstream of the GC-rich RNA element in SEQ ID NO: 4 or SEQ ID NO: 60. In some aspects, the structural RNA element is inserted 30-40 nucleotides upstream of the GC-rich RNA element in SEQ ID NO: 4 or SEQ ID NO: 60.

In any of the foregoing aspects, an mRNA comprises a 5′ cap, a 5′ UTR comprising a structural RNA element, an ORF encoding a polypeptide, and a 3′ UTR comprising a nucleotide sequence at least 70%, at least 75%, at least 80%, at least 85%, at least 90%, at least 95%, at least 96%, at least 97%, at least 98%, at least 99% identical or 100% identical to a nucleotide sequence of SEQ ID NO: 76 or SEQ ID NO: 78, wherein the structural RNA element is inserted upstream of and immediately adjacent to the GC-rich RNA element in SEQ ID NO: 4 or SEQ ID NO: 60.

In any of the foregoing aspects, an mRNA comprises a 5′UTR comprising a C-rich RNA element that is inserted proximal to the 5′ cap of the mRNA in SEQ ID NO: 4 or SEQ ID NO: 60, wherein the C-rich RNA element comprises a nucleotide sequence selected from selected from the group consisting of SEQ ID NO: 31, SEQ ID NO: 32 and SEQ ID NO: 33. In some aspects, the C-rich RNA element comprises the nucleotide sequence of SEQ ID NO: 31. In some aspects, the C-rich RNA element is inserted about 1-10 nucleotides downstream of the 5′ cap in SEQ ID NO: 4 or SEQ ID NO: 60. In some aspects, the C-rich RNA element comprises the nucleotide sequence of SEQ ID NO: 31. In some aspects, the C-rich RNA element is inserted about 1-10 nucleotides downstream of the 5′ cap in SEQ ID NO: 4 or SEQ ID NO: 60. In some aspects, the C-rich RNA element is inserted about 5-10 nucleotides downstream of the 5′ cap in SEQ ID NO: 4 or SEQ ID NO: 60. In some aspects, the C-rich RNA element is inserted about 1-6 nucleotides downstream of the 5′ cap of in SEQ ID NO: 4 or SEQ ID NO: 60. In some aspects, the C-rich RNA element is downstream of and immediately adjacent to a transcription start site element in the 5′UTR, wherein the transcription start site element comprises the nucleotide sequence [5′-GGGAAA-3′] in SEQ ID NO: 4 or the nucleotide sequence [5′-AGGAAA-3′] in SEQ ID NO: 60.

In some aspects, the disclosure provides an mRNA, wherein the mRNA comprises: a 5′ cap, a 5′ UTR, wherein the 5′ UTR comprises a nucleotide sequence selected from the group consisting of: SEQ ID NO: 116; SEQ ID NO: 120; SEQ ID NO: 124; SEQ ID NO: 41; and SEQ ID NO: 128, an ORF encoding a polypeptide, and a 3′ UTR, wherein the 3′UTR comprises a nucleotide sequence at least 70%, at least 75%, at least 80%, at least 85%, at least 90%, at least 95%, at least 96%, at least 97%, at least 98%, at least 99% identical or 100% identical to a nucleotide sequence of SEQ ID NO: 76 or SEQ ID NO: 78. In some aspects, the 3′UTR comprises one or more microRNA (miRNA) binding sites. In some aspects, the 3′UTR comprises 1, 2, 3, 4, 5, 6, 7, 8, 9, or 10 miRNA binding site(s). In some aspects, the 3′UTR comprises 1, 2, 3 or 4 miRNA binding sites. In some aspects, the miRNA binding site is targeted by miR-142-3p or miR-142-5p. In some aspects, the miRNA binding site comprises a nucleotide sequence at least 80%, at least 85%, at least 90%, at least 95%, at least 96%, at least 97%, at least 98%, at least 99%, or 100% identical to the nucleotide sequence of SEQ ID NO: 179 or SEQ ID NO: 181. In some aspects, the miRNA binding site comprises the nucleotide sequence of SEQ ID NO: 179. In some aspects, the miRNA binding site comprises the nucleotide sequence of SEQ ID NO: 181.

In any of the foregoing aspects, an mRNA comprises a 5′ cap, a 5′ UTR, wherein the 5′ UTR comprises a nucleotide sequence selected from the group consisting of: SEQ ID NO: 116; SEQ ID NO: 120; SEQ ID NO: 124; SEQ ID NO: 41; and SEQ ID NO: 128, an ORF encoding a polypeptide, and a 3′UTR comprising a nucleotide sequence at least 70%, at least 75%, at least 80%, at least 85%, at least 90%, at least 95%, at least 96%, at least 97%, at least 98%, at least 99% identical or 100% identical to a nucleotide sequence of SEQ ID NO: 76 or SEQ ID NO: 78, wherein the 3′UTR comprises one or more stop codons at the 5′end of the 3′UTR, and wherein the 3′UTR comprises 1, 2, 3, or 4 miRNA binding sites located proximal to the one or more stop codons. In some aspects, the miRNA binding site(s) are located downstream of and immediately adjacent to the one or more stop codons at the 5′end of the 3′UTR. In some aspects, the miRNA binding sites are located about 45-50, about 40-45, about 35-40, about 30-35, about 25-30, about 20-25, about 15-20, about 10-15, about 6-10 nucleotides, about 1-5 nucleotide(s), or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) downstream of the one or more stop codons at the 5′end of the 3′UTR. In some aspects, the miRNA binding sites are located about 10, about 9, about 8, about 7, about 6, about 5, about 4, about 3, about 2, or about 1 nucleotide(s) downstream of the one or more stop codons at the 5′end of the 3′UTR.

In any of the foregoing aspects, an mRNA comprises a 5′ cap, a 5′ UTR, wherein the 5′ UTR comprises a nucleotide sequence selected from the group consisting of: SEQ ID NO: 116; SEQ ID NO: 120; SEQ ID NO: 124; SEQ ID NO: 41; and SEQ ID NO: 128, an ORF encoding a polypeptide, and a 3′UTR comprising a nucleotide sequence at least 70%, at least 75%, at least 80%, at least 85%, at least 90%, at least 95%, at least 96%, at least 97%, at least 98%, at least 99% identical or 100% identical to a nucleotide sequence of SEQ ID NO: 76 or SEQ ID NO: 78, wherein the 3′UTR comprises 1, 2, 3, or 4 miRNA binding sites located proximal to the 3′end of the 3′UTR. In some aspects, the miRNA binding site(s) are located upstream of and immediately adjacent to the 3′end of the 3′UTR. In some aspects, the miRNA binding site(s) are located about 1-5, about 6-10, about 10-15, about 15-20, about 20-25, about 25-30, about 30-35, about 35-40, about 40-45, or about 45-50 nucleotide(s) or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotide(s) upstream of the 3′end of the 3′UTR. In some aspects, the miRNA binding site(s) are located about 1, about 2, about 3, about 4, or about 5, about 6, about 7, about 8, about 9 or about 10 nucleotide(s) upstream of the 3′end of the 3′UTR.

In any of the foregoing aspects, an mRNA comprises a 5′ cap, a 5′ UTR, wherein the 5′ UTR comprises a nucleotide sequence selected from the group consisting of: SEQ ID NO: 116; SEQ ID NO: 120; SEQ ID NO: 124; SEQ ID NO: 41; and SEQ ID NO: 128, an ORF encoding a polypeptide, and a 3′UTR comprising a nucleotide sequence at least 70%, at least 75%, at least 80%, at least 85%, at least 90%, at least 95%, at least 96%, at least 97%, at least 98%, at least 99% identical or 100% identical to a nucleotide sequence of SEQ ID NO: 76 or SEQ ID NO: 78, wherein the 3′UTR comprises 1, 2, 3, 4, 5, 6, 7, 8, 9, or 10 miRNA binding sites. In some aspects, the 3′UTR comprises 1, 2, 3, 4, 5, 6, 7, 8, 9, or 10 miRNA binding sites, wherein an upstream miRNA binding site is located directly adjacent to one or more downstream miRNA binding site(s). In some aspects, the 3′UTR comprises 1, 2, 3, 4, 5, 6, 7, 8, 9, or 10 miRNA binding sites, wherein an upstream miRNA binding site is separated from a downstream miRNA binding site by about 1-5, about 1-10, about 5-10, about 5-15, about 10-20, about 15-20, about 15-30, or about 20-30 nucleotide(s) or about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotide(s). In some aspects, the 3′UTR comprises 1, 2, 3, 4, 5, 6, 7, 8, 9, or 10 miRNA binding sites, an upstream miRNA binding site is separated from a downstream miRNA binding site by about 1, about 2, about 3, about 4, about 5, about 6, about 7, about 8, about 9 or about 10 nucleotide(s).

In any of the foregoing aspects, an mRNA comprises a 5′ cap, a 5′ UTR, wherein the 5′ UTR comprises a nucleotide sequence selected from the group consisting of: SEQ ID NO: 116; SEQ ID NO: 120; SEQ ID NO: 124; SEQ ID NO: 41; and SEQ ID NO: 128, an ORF encoding a polypeptide, and a 3′UTR comprising a nucleotide sequence at least 70%, at least 75%, at least 80%, at least 85%, at least 90%, at least 95%, at least 96%, at least 97%, at least 98%, at least 99% identical or 100% identical to a nucleotide sequence of SEQ ID NO: 78, wherein the 3′UTR comprises 1, 2, 3, or 4 miR-142-3p binding sites, and wherein the miR-142-3p binding site comprises the nucleotide sequence of SEQ ID NO: 179. In some aspects, the 1, 2, 3, or 4 miR-142-3p binding sites are located proximal to the 3′end or the 3′UTR. In some aspects, the 3′UTR comprises the nucleotide sequence of SEQ ID NO: 170. In some aspects, the 3′UTR comprises one or more stop codons at the 5′end and wherein the 1, 2, 3, or 4 miR-142-3p binding sites are located proximal to the one or more stop codons. In some aspects, the 3′UTR comprises the nucleotide sequence of SEQ ID NO: 172.

In any of the foregoing aspects, an mRNA comprises a 5′ cap, a 5′ UTR, wherein the 5′ UTR comprises a nucleotide sequence selected from the group consisting of: SEQ ID NO: 116; SEQ ID NO: 120; SEQ ID NO: 124; SEQ ID NO: 41; and SEQ ID NO: 128, an ORF encoding a polypeptide, and a 3′UTR comprising a nucleotide sequence at least 70%, at least 75%, at least 80%, at least 85%, at least 90%, at least 95%, at least 96%, at least 97%, at least 98%, at least 99% identical or 100% identical to a nucleotide sequence of SEQ ID NO: 76, wherein the 3′UTR comprises 1, 2, 3, or 4 miR-142-3p binding sites, and wherein the miR-142-3p binding site comprises the nucleotide sequence of SEQ ID NO: 179. In some aspects, the 1, 2, 3, or 4 miR-142-3p binding sites are located proximal to the 3′end or the 3′UTR. In some aspects, the 3′UTR comprises the nucleotide sequence of SEQ ID NO: 174. In some aspects, the 3′UTR comprises one or more stop codons at the 5′end and wherein the 1, 2, 3, or 4 miR-142-3p binding sites are located proximal to the one or more stop codons. In some aspects, the 3′UTR comprises the nucleotide sequence of SEQ ID NO: 176.

In some aspects, the disclosure provides an mRNA, wherein the mRNA comprises: a 5′ cap, a 5′ UTR, wherein the 5′ UTR comprises a nucleotide sequence selected from the group consisting of: SEQ ID NO: 116; SEQ ID NO: 120; SEQ ID NO: 124; SEQ ID NO: 41; and SEQ ID NO: 128, an ORF encoding a polypeptide, and a 3′UTR, wherein the 3′UTR comprises a nucleotide sequence selected from the group consisting of: SEQ ID NO: 170, SEQ ID NO: 172, SEQ ID NO: 174, and SEQ ID NO: 176.

In some aspects, the disclosure provides an mRNA, wherein the mRNA comprises: a 5′ cap, a 5′ UTR, an ORF encoding a polypeptide, and a 3′ UTR, wherein the 5′ UTR and 3′ UTR are selected from the group consisting of: the nucleotide sequence of SEQ ID NO: 120 and the nucleotide sequence of SEQ ID NO: 170; the nucleotide sequence of SEQ ID NO: 120 and the nucleotide sequence of SEQ ID NO: 172; the nucleotide sequence of SEQ ID NO: 120 and the nucleotide sequence of SEQ ID NO: 174; the nucleotide sequence of SEQ ID NO: 120 and the nucleotide sequence of SEQ ID NO: 176; the nucleotide sequence of SEQ ID NO: 41 and the nucleotide sequence of SEQ ID NO: 170; the nucleotide sequence of SEQ ID NO: 41 and the nucleotide sequence of SEQ ID NO: 172; the nucleotide sequence of SEQ ID NO: 41 and the nucleotide sequence of SEQ ID NO: 174; the nucleotide sequence of SEQ ID NO: 41 and the nucleotide sequence of SEQ ID NO: 176; the nucleotide sequence of SEQ ID NO: 128 and the nucleotide sequence of SEQ ID NO: 170; the nucleotide sequence of SEQ ID NO: 128 and the nucleotide sequence of SEQ ID NO: 172; the nucleotide sequence of SEQ ID NO: 128 and the nucleotide sequence of SEQ ID NO: 174; and the nucleotide sequence of SEQ ID NO: 128 and the nucleotide sequence of SEQ ID NO: 176. In some aspects, the 5′ UTR and 3′ UTR are selected from the group consisting of: the nucleotide sequence of SEQ ID NO: 120 and the nucleotide sequence of SEQ ID NO: 170, the nucleotide sequence of SEQ ID NO: 120 and the nucleotide sequence of SEQ ID NO: 172.

In any of the foregoing aspects, an mRNA comprises a 5′ cap, a 5′UTR, an initiation codon, a full open reading frame encoding a polypeptide, and a 3′ UTR, wherein the mRNA comprises at least one chemically modified nucleoside. In some aspects, the at least one chemically modified nucleoside is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine. In some aspects, at least about 10%, at least about 20%, at least about 30%, at least about 40%, at least about 50%, at least about 60%, at least about 70%, at least about 80%, at least about 90%, at least about 95%, at least about 99%, or about 100% of the nucleosides comprising the mRNA comprise the at least one chemically modified nucleoside. In some aspects, the at least one chemically modified nucleoside is N1-methylpseudouridine, and wherein at least 80%, at least 85%, at least 90%, at least 95%, at least 99%, or 100% of the uracil nucleotides are N1-methylpseudouridine. In some aspects, the mRNA is fully modified with N1-methylpseudouridine. In some aspects, the at least one modified nucleoside is 5-methoxyuridine. In some aspects, at least 95% of uracil nucleotides comprising the ORF comprise 5-methoxyuridine, and wherein the uracil content in the ORF is between about 100% and about 150% of the theoretical minimum. In some aspects, the mRNA is fully modified with 5-methoxyuridine.

In any of the foregoing aspects, an mRNA comprises a 5′ cap, a 5′UTR, an initiation codon, a full open reading frame encoding a polypeptide, and a 3′ UTR, wherein the mRNA comprises a poly A tail.

In any of the foregoing aspects, an mRNA comprises a 5′ cap, a 5′UTR, an initiation codon, a full open reading frame encoding a polypeptide, and a 3′ UTR, wherein the mRNA comprises a 5′Cap 1 structure.

In any of the foregoing aspects, an mRNA comprises a 5′ cap, a 5′UTR, an initiation codon, a full open reading frame encoding a polypeptide, and a 3′ UTR, wherein an expression level and/or an activity of the polypeptide translated from the mRNA is increased relative to an mRNA that does not comprise the 5′ UTR, 3′ UTR, or a combination thereof.

In some aspects, the disclosure provides a pharmaceutical composition comprising an mRNA of the disclosure and a pharmaceutically acceptable carrier.

In some aspects, the disclosure provides a lipid nanoparticle comprising an mRNA of the disclosure. In some aspects, the lipid nanoparticle comprises an ionizable lipid, a sterol, a phospholipid, and a polyethylene glycol lipid.

In some aspects, the disclosure provides a pharmaceutical composition comprising a lipid nanoparticle comprising an mRNA of the disclosure and a pharmaceutically acceptable carrier.

In any of the foregoing aspects, a pharmaceutical composition of the disclosure or lipid nanoparticle of the disclosure is used in treating or delaying progression of a disease or disorder in a subject in need thereof.

In any of the foregoing aspects, a pharmaceutical composition of the disclosure or lipid nanoparticle of the disclosure is used in the manufacture of a medicament for treating or delaying progression of a disease or disorder in a subject in need thereof.

In some aspects, the disclosure provides a kit comprising a container comprising an mRNA of the disclosure, a pharmaceutical composition of the disclosure or lipid nanoparticle of the disclosure and a package insert comprising instructions for administration of the mRNA, the pharmaceutical composition of lipid nanoparticle, for treating or delaying progression of a disease or disorder in a subject.

In some aspects, the disclosure provides a method of treating or delaying progression of a disease or disorder in a subject in need thereof, the method comprising administering an mRNA of the disclosure, a pharmaceutical composition of the disclosure, or a lipid nanoparticle of the disclosure, thereby treating or delaying progression of the disease or disorder in the subject.

### BRIEF DESCRIPTION OF THE DRAWINGS

FIGS. 1A-1B provide bar graphs showing the expression level (FIG. 1A) and activity (FIG. 1B) of a cellular enzyme (enzyme_A) in hepatocytes harvested from mice deficient in enzyme_A and transfected in vitro with enzyme_A-encoding mRNA constructs comprising a 5′UTR encoding an RNAse P stem loop.

FIGS. 2A-2B provides dot plots showing leaky scanning plotted against the level of mRNA expression for reporter mRNA transfected in HeLa cells (FIG. 2A) and AML12 cells (FIG. 2B). mRNAs comprised different length 5′UTRs (i.e., white points are short 5′UTRs and black points are long 5′UTRs). Those identified by name have 5′UTRs that comprised multiple RNA elements, including an RNAse P stem loop (F593, F153, RNAse P_p1, 5′v1.1).

FIGS. 3A-3B provide bar graphs showing the expression level (FIG. 3A) and activity (FIG. 3B) of a cellular enzyme (enzyme_A) in hepatocytes harvested from mice deficient in enzyme_A and transfected in vitro with enzyme_A-encoding mRNA constructs comprising a 3′UTR derived from the human MRPS12 gene (rps12 3′UTR), from the mouse Sod2 gene (sod2 3′UTR), or the human OXA1L gene (oxal 3′UTR).

FIGS. 4A-4B provide graphs showing the expression level (FIG. 4A) and activity (FIG. 4B) of a cellular enzyme (enzyme_B) in hepatocytes harvested from mice deficient in enzyme_B following treatment with enzyme_B-encoding mRNA constructs comprising varied 3′UTRs. Shown in FIG. 4B is the enzymatic activity of enzyme_B in mouse liver lysates harvested on day 15, which was 24 hours post 2^{nd} dose of mRNA (mice were dosed on day 0 and day 14).

FIGS. 5A-5D provide graphs showing the plasma concentration of a biomarker of enzyme_B enzymatic activity (enzyme_B-BM1) in enzyme_B deficient mice following treatment with mRNA encoding enzyme_B and comprising varied 3′UTRs (3′v1.1, 3′rps12, 3′sod2). mRNA was administered on day 0 and day 14. Plasma concentration of enzyme_B-BM1 was determined by liquid chromatography-tandem mass spectrometry (LC-MS/MS) in samples isolated on day 1 (FIG. 5A), day 7 (FIG. 5B), day 14 (FIG. 5C) and day 15 (FIG. 5D) post-mRNA administration. FIG. 5E provides a graph showing the mean concentration of plasma enzyme_B-BM1 for each treatment group over time, as indicated.

FIGS. 6A-6E provide graphs showing the concentration of enzyme_B-BM2 (a second biomarker of enzyme_B enzymatic activity) as determined by LC-MS/MS in plasma from mice as treated in FIGS. 5A-5E.

FIG. 7 provides a graph showing the level of luciferin expression in wild type mice measured by bioluminescence imaging (BLI) following treatment with mRNA encoding luciferin.

FIG. 8 provides a graph showing expression of erythropoietin (EPO) in wild type mice measured following treatment with mRNA encoding EPO.

FIGS. 9A-9C provide graphs showing weight loss in enzyme_A-deficient mice following treatment with enzyme_A-encoding mRNA constructs. mRNA was administered on day 0 and day 31.

FIGS. 9A-9C show percent change in body weight on days 14, 21, and 28 post-mRNA administration respectively. FIG. 9D provides a graph showing the average percent change in body weight over time.

FIGS. 10A-10C provide graphs showing the plasma concentration of a biomarker of enzyme_A activity (i.e., enzyme_A-BM2) in mice treated as in FIGS. 9A-9D. Shown in FIGS. 10A-10C is the plasma concentration of enzyme_A-BM2 on day 16, 21, and 28 respectively.

FIGS. 11A-11C provide graphs showing the amount of enzyme_A protein (FIG. 11A), enzyme_A activity (FIG. 11B), and enzyme_A-encoding mRNA (FIG. 11C) in liver lysates isolated on day 32, corresponding to 24 h after the 2^{nd} dose from mice treated as in FIGS. 9A-9C.

FIGS. 12A-12B provides graphs showing expression level of enzyme_B (FIG. 12A) and enzyme_B activity (FIG. 12B) in liver lysates harvested from wild type mice following administration of mRNAs encoding enzyme_B.

FIG. 13A provides an image of an immunoblot prepared from liver lysates harvested from enzyme_B deficient mice following administration of mRNAs encoding enzyme_B in different lipid nanoparticle formulations with staining for enzyme_B protein and an endogenous control protein. FIG. 13B-13C provide graphs showing enzyme_B protein expression level in liver lysates harvested from enzyme_B deficient mice following administration of mRNAs encoding enzyme_B in different lipid nanoparticle formulations measured by quantitative immunoblot (FIG. 13B) and LC-MS (FIG. 13C).

FIG. 14A provides a graph showing relative enzyme_B protein expression level and activity measured in liver lysates harvested from enzyme_B deficient mice following administration of mRNAs encoding enzyme_B in different lipid nanoparticle formulations. FIG. 14B provides the activity of enzyme_B in liver lysates as in FIG. 14A, with activity provided in units of nmol/min/mg protein.

FIG. 15A-15B provides graphs showing levels of enzyme_B-BM1 in plasma (FIG. 15A) and in tissue lysates of liver, kidney and heart (FIG. 15B) collected at one day following administration of mRNA in different lipid nanoparticle formulations to enzyme_B-deficient mice.

### DETAILED DESCRIPTION

Treatment with an mRNA encoding a therapeutic polypeptide of interest has numerous clinical, prophylactic, and therapeutic applications for treating or delaying progression of a disease or disorder in an individual. Improving the expression level and/or the activity of an encoded therapeutic polypeptide is desirable for use of therapeutic mRNAs in such applications. Without being bound by theory, it is believed that certain mRNA chemical and/or structural modifications that function to regulate the post-transcriptional stability, localization, and/or translation of the mRNA can yield increased expression and/or activity of an encoded polypeptide of interest.

Accordingly, the present disclosure provides mRNAs (e.g., modified mRNAs) encoding a polypeptide of interest and comprising a heterologous NEMP-derived 3′UTR, a 5′UTR comprising one or more functional RNA elements (e.g., an RNAse P stem loop), or a combination thereof that enhance protein expression and/or activity, as well as compositions (e.g., lipid nanoparticles) and methods thereof (e.g., methods for treating a mitochondrial disease). In some embodiments, the 3′UTR is derived from a naturally-occurring RNA. In some embodiments, the 3′UTR comprises a nucleotide sequence that is substantially identical (e.g., about 50%, 60%, 70%, 80%, 90% or about 100% identical) to the nucleotide sequence of a 3′UTR of an mRNA encoding a NEMP. In some embodiments, the functional RNA element comprises a nucleotide sequence that is substantially identical (e.g., about 50%, 60%, 70%, 80%, 90% or about 100% identical) to the nucleotide sequence of a stem-loop that comprises the RNA component of the nuclear RNAse P (RNAse P) ribonucleoprotein complex or the mitochondrial RNAse P (MRP) ribonucleoprotein complex.

In some embodiments, an mRNA of the disclosure comprises a 5′UTR comprising one or more functional RNA elements (e.g., an RNAse P stem-loop), optionally in combination with a NEMP-derived 3′UTR described herein. In some embodiments, the mRNAs of the disclosure comprise both a NEMP-derived 3′UTR and a 5′UTR comprising one or more functional RNA elements (e.g., an RNAse P stem-loop). In some embodiments, the mRNA of the disclosure comprises an ORF which encodes a mitochondrial-targeting sequence (MTS). In some embodiments, the mRNAs of the disclosure comprise a lipid nanoparticle.

In some embodiments, the NEMP-derived 3′UTR and/or 5′UTR comprising one or more functional RNA elements (e.g., an RNAse P stem-loop) function to regulate mRNA stability (e.g., increase mRNA half-life), to regulate mRNA cellular localization, to provide a desired translational regulatory activity, or any combination thereof. In some embodiments, the NEMP-derived 3′UTR and/or 5′UTR comprising one or more functional RNA elements (e.g., an RNAse P stem-loop) function to enhance the expression and/or activity of a polypeptide of interest encoded by the mRNA.

### Polynucleotides Comprising Functional RNA Elements in the 5′UTR

The present disclosure provides synthetic polynucleotides comprising a modification (e.g., an RNA element), wherein the modification provides a desired translational regulatory activity. In some embodiments, the disclosure provides a polynucleotide comprising a 5′ untranslated region (UTR), an initiation codon, a full open reading frame encoding a polypeptide, a 3′ UTR, and at least one modification, wherein the at least one modification provides a desired translational regulatory activity, for example, a modification that promotes and/or enhances the translational fidelity of mRNA translation. In some embodiments, the disclosure provides a polynucleotide comprising a 5′cap, a 5′ untranslated region (UTR), a Kozak-like sequence, an initiation codon, a full open reading frame encoding a polypeptide, a 3′ UTR, and at least one modification, wherein the at least one modification provides a desired translational regulatory activity, for example, a modification that promotes and/or enhances the translational fidelity of mRNA translation.

In some embodiments, the desired translational regulatory activity is a cis-acting regulatory activity. In some embodiments, the desired translational regulatory activity is an increase in the residence time of the 43S pre-initiation complex (PIC) or ribosome at, or proximal to, the initiation codon. In some embodiments, the desired translational regulatory activity is an increase in the initiation of polypeptide synthesis at or from the initiation codon. In some embodiments, the desired translational regulatory activity is an increase in the amount of polypeptide translated from the full open reading frame. In some embodiments, the desired translational regulatory activity is an increase in the fidelity of initiation codon decoding by the PIC or ribosome. In some embodiments, the desired translational regulatory activity is inhibition or reduction of leaky scanning by the PIC or ribosome. In some embodiments, the desired translational regulatory activity is a decrease in the rate of decoding the initiation codon by the PIC or ribosome. In some embodiments, the desired translational regulatory activity is inhibition or reduction in the initiation of polypeptide synthesis at any codon within the mRNA other than the initiation codon. In some embodiments, the desired translational regulatory activity is inhibition or reduction of the amount of polypeptide translated from any open reading frame within the mRNA other than the full open reading frame. In some embodiments, the desired translational regulatory activity is inhibition or reduction in the production of aberrant translation products. In some embodiments, the desired translational regulatory activity is an increase in ribosomal density on the mRNA. In some embodiments, the desired translational regulatory activity is a combination of one or more of the foregoing translational regulatory activities.

Accordingly, the present disclosure provides a polynucleotide, e.g., an mRNA, comprising an RNA element that comprises a sequence and/or an RNA secondary structure(s) that provides a desired translational regulatory activity as described herein. In some aspects, the mRNA comprises an RNA element that comprises a sequence and/or an RNA secondary structure(s) that promotes and/or enhances the translational fidelity of mRNA translation. In some aspects, the mRNA comprises an RNA element that comprises a sequence and/or an RNA secondary structure(s) that provides a desired translational regulatory activity, such as inhibiting and/or reducing leaky scanning. In some aspects, the disclosure provides an mRNA that comprises an RNA element that comprises a sequence and/or an RNA secondary structure(s) that inhibits and/or reduces leaky scanning thereby promoting the translational fidelity of the mRNA.

In some embodiments, the RNA element comprises natural and/or modified nucleotides. In some embodiments, the RNA element comprises of a sequence of linked nucleotides, or derivatives or analogs thereof, that provides a desired translational regulatory activity as described herein. In some embodiments, the RNA element comprises a sequence of linked nucleotides, or derivatives or analogs thereof, that forms or folds into a stable RNA secondary structure, wherein the RNA secondary structure provides a desired translational regulatory activity as described herein. RNA elements can be identified and/or characterized based on the primary sequence of the element (e.g., GC-rich element and/or C-rich element), by RNA secondary structure formed by the element (e.g. stem-loop), by the location of the element within the RNA molecule (e.g., located within the 5′ UTR of an mRNA), by the biological function and/or activity of the element (e.g., “translational enhancer element”), and any combination thereof.

### Structural RNA Elements

In some aspects, the disclosure provides an mRNA comprising at least one or more structural RNA element(s) comprising a sequence of linked ribonucleotides that folds into a hairpin or stem-loop structure that provides a translational regulatory activity as described herein. As described in the Examples, a structural RNA element derived from human H1 RNA comprising a nucleotide sequence of 20 nucleotides in length and forming a stem-loop was unexpectedly shown to promote and/or enhance the translational fidelity of polypeptides encoded by mRNAs with 5′ UTRs comprising the element.

Accordingly, in some aspects the disclosure provides mRNAs comprising a 5′ UTR comprising at least one or more structural RNA element(s) comprising a stem-loop. In some embodiments, the structural RNA element comprising a stem-loop comprises a nucleotide sequence of about 10-30 nucleotides, about 15-25 nucleotides, about 20-25 nucleotides, about 30, 29, 28, 27, 26, 25, 24, 23, 22, 21, 20, 19, 18, 17, 16, 15, 14, 13, 12, or about 10 nucleotides in length. In some embodiments, the structural RNA element comprising a stem-loop comprises a nucleotide sequence of about 20 nucleotides.

In some embodiments, the structural RNA element comprises a stem-loop comprising a double-stranded stem comprising about 3-8 base pairs, about 4-7 base pairs, about 5-6 base pairs, about 3, 4, 5, 6, 7, 8 base pairs. In some embodiments, the double-stranded stem comprises about 4-7 base pairs. In some embodiments, the double-stranded stem comprises about 4 base pairs. In some embodiments, the double-stranded stem comprises about 7 base pairs.

In some embodiments, the structural RNA element comprises a stem-loop comprising a single-stranded loop of about 3-8 nucleotides, about 4-7 nucleotides, about 5-6 nucleotides, about 3, 4, 5, 6, 7, or 8 nucleotides in length. In some embodiments, the single-stranded loop is about 5 nucleotides in length.

In some embodiments, the structural RNA element comprises a stem-loop, wherein the stem-loop has a deltaG (ΔG) of about −30 kcal/mol, about −20 to −30 kcal/mol, about −20 kcal/mol, about −10 to −20 kcal/mol, about −10 kcal/mol, or about −5 to −10 kcal/mol.

In some embodiments, the structural RNA element comprising a stem-loop is located upstream of a Kozak-like sequence in the 5′ UTR. In some embodiments, the structural RNA element comprising a stem-loop is located upstream of and immediately adjacent to a Kozak-like sequence in the 5′ UTR. In some embodiments, the structural RNA element comprising a stem-loop is located about 45-50, about 40-45, about 35-40, about 30-35, about 25-30, about 20-25, about 15-20, about 10-15, about 6-10 nucleotides, about 1-5 nucleotide(s), or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) upstream of a Kozak-like sequence in the 5′ UTR. In some embodiments, the structural RNA element comprising a stem-loop is located about 50, about 45, about 40, about 35, about 30, about 25, about 20, about 15, about 10 or about 5, about 4, about 3, about 2, or about 1 nucleotide(s) upstream of a Kozak-like sequence in the 5′ UTR. In some embodiments, the structural RNA element comprising a stem-loop is located about 5, about 4, about 3, about 2, or about 1 nucleotide(s) upstream of a Kozak-like sequence in the 5′ UTR. In some embodiments, the structural RNA element comprising a stem-loop is located 10 nucleotides upstream of a Kozak-like sequence in the 5′ UTR. In some embodiments, the structural RNA element comprising a stem-loop is located 45 nucleotides upstream of a Kozak-like sequence in the 5′ UTR. In some embodiments, the structural RNA element comprising a stem-loop is located 28 nucleotides upstream of a Kozak-like sequence in the 5′ UTR.

In some embodiments, the structural RNA element comprising a stem-loop is located downstream of the 5′ cap or 5′ end of the mRNA in the 5′ UTR. In some embodiments, the structural RNA element comprising a stem-loop is located downstream of and immediately adjacent to the 5′ cap or 5′ end of the mRNA in the 5′ UTR. In some embodiments, the structural RNA element comprising a stem-loop is located about 45-50, about 40-45, about 35-40, about 30-35, about 25-30, about 20-25, about 15-20, about 10-15, about 6-10 nucleotides, about 1-5 nucleotide(s), or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) downstream of the 5′ cap or 5′ end of the mRNA in the 5′ UTR. In some embodiments, the structural RNA element comprising a stem-loop is located about 50, about 45, about 40, about 35, about 30, about 25, about 20, about 15, about 10 or about 5, about 4, about 3, about 2, or about 1 nucleotide(s) downstream of the 5′ cap or 5′ end of the mRNA in the 5′ UTR. In some embodiments, the structural RNA element comprising a stem-loop is located about 5, about 4, about 3, about 2, or about 1 nucleotide(s) downstream of the 5′ cap or 5′ end of the mRNA in the 5′ UTR. In some embodiments, the structural RNA element comprising a stem-loop is located 41 nucleotides downstream of the 5′ cap or 5′ end of the mRNA in the 5′ UTR. In some embodiments, the structural RNA element comprising a stem-loop is located 6 nucleotides downstream of the 5′ cap or 5′ end of the mRNA in the 5′ UTR. In some embodiments, the structural RNA element comprising a stem-loop is located 23 nucleotides downstream of the 5′ cap or 5′ end of the mRNA in the 5′ UTR.

Stem-loop structures that function as structural RNA elements have been identified for human H1 RNA and MRP ribonucleoprotein as described by Wang, G. et al (2010) Cell 142:456-467, which is incorporated herein in its entirety. In some embodiments, the structural RNA element comprising a stem-loop comprises a nucleotide sequence that is about 80%, 85%, 90%, 95%, 96%, 97%, 98%, 99% or 100% identical to a nucleotide sequence comprising a human H1 RNA stem-loop structure. In some embodiments, the structural RNA element comprising a stem-loop comprises a nucleotide sequence that is about 80%, 85%, 90%, 95%, 96%, 97%, 98%, 99% or 100% identical to a nucleotide sequence comprising a stem-loop structure of the RNA component of the MRP ribonucleoprotein complex.

In some embodiments, the structural RNA element comprising a stem-loop comprises a nucleotide sequence selected from the group consisting of: SEQ ID NO: 6 and SEQ ID NO: 47. In some embodiments, the structural RNA element comprising a stem-loop comprises the nucleotide sequence of SEQ ID NO: 6. In some embodiments, the structural RNA element comprising a stem-loop comprises the nucleotide sequence of SEQ ID NO: 47.

In some embodiments, the structural RNA element comprising a stem-loop comprises a nucleotide sequence that is about 80%, about 85%, about 90%, about 95%, about 96%, about 97%, about 98%, about 99%, or about 100% identical to a nucleotide sequence selected from the group consisting of: SEQ ID NO: 6 and SEQ ID NO: 47. In some embodiments, the structural RNA element comprising a stem-loop comprises a nucleotide sequence that is about 80%, about 85%, about 90%, about 95%, about 96%, about 97%, about 98%, about 99%, or about 100% identical to the nucleotide sequence of SEQ ID NO: 6. In some embodiments, the structural RNA element comprising a stem-loop comprises a nucleotide sequence that is about 80%, about 85%, about 90%, about 95%, about 96%, about 97%, about 98%, about 99%, or about 100% identical to the nucleotide sequence of SEQ ID NO: 47.

In some embodiments, the structural RNA element comprising a stem-loop comprises a nucleotide sequence that is about 80%, about 85%, about 90%, about 95%, about 96%, about 97%, about 98%, about 99%, or about 100% identical to a nucleotide sequence identified by SEQ ID NO: 6, wherein the stem-loop has a deltaG (ΔG) that is at least 50%, at least 60%, at least 70%, at least 80%, at least 90%, at least 95%, at least 96%, at least 97%, at least 98%, at least 99%, or 100% of the deltaG (ΔG) of the stem-loop identified by SEQ ID NO: 6. In some embodiments, the structural RNA element comprising a stem-loop comprises a nucleotide sequence that is about 80%, about 85%, about 90%, about 95%, about 96%, about 97%, about 98%, about 99%, or 100% identical to a nucleotide sequence identified by SEQ ID NO: 6, wherein the stem-loop has a deltaG (ΔG) of about −30 kcal/mol, about −20 to −30 kcal/mol, about −20 kcal/mol, about −10 to −20 kcal/mol, about −10 to −15 kcal/mol, about −12 kcal/mol, about −10 kcal/mol, or about −5 to −10 kcal/mol. In some embodiments, the structural RNA element comprising a stem-loop comprises a nucleotide sequence that is about 80%, about 85%, about 90%, about 95%, about 96%, about 97%, about 98%, about 99%, or 100% identical to a nucleotide sequence identified by SEQ ID NO: 6, wherein the stem-loop has a deltaG (ΔG) of about −10 kcal/mol. In some embodiments, the structural RNA element comprising a stem-loop comprises a nucleotide sequence that is about 80%, about 85%, about 90%, about 95%, about 96%, about 97%, about 98%, about 99%, or 100% identical to a nucleotide sequence identified by SEQ ID NO: 6, wherein the stem-loop has a deltaG (ΔG) of about −11 kcal/mol. In some embodiments, the structural RNA element comprising a stem-loop comprises a nucleotide sequence that is about 80%, about 85%, about 90%, about 95%, about 96%, about 97%, about 98%, about 99%, or 100% identical to a nucleotide sequence identified by SEQ ID NO: 6, wherein the stem-loop has a deltaG (ΔG) of about −12 kcal/mol. In some embodiments, the structural RNA element comprising a stem-loop comprises a nucleotide sequence that is about 80%, about 85%, about 90%, about 95%, about 96%, about 97%, about 98%, about 99%, or 100% identical to a nucleotide sequence identified by SEQ ID NO: 6, wherein the stem-loop has a deltaG (ΔG) of about −13 kcal/mol. In some embodiments, the structural RNA element comprising a stem-loop comprises a nucleotide sequence that is about 80%, about 85%, about 90%, about 95%, about 96%, about 97%, about 98%, about 99%, or 100% identical to a nucleotide sequence identified by SEQ ID NO: 6, wherein the stem-loop has a deltaG (ΔG) of about −14 kcal/mol. In some embodiments, the structural RNA element comprising a stem-loop comprises a nucleotide sequence that is about 80%, about 85%, about 90%, about 95%, about 96%, about 97%, about 98%, about 99%, or 100% identical to a nucleotide sequence identified by SEQ ID NO: 6, wherein the stem-loop has a deltaG (ΔG) of about −15 kcal/mol.

In some embodiments, the structural RNA element comprising a stem-loop comprises a nucleotide sequence that is about 80%, about 85%, about 90%, about 95%, about 96%, about 97%, about 98%, about 99%, or 100% identical to a nucleotide sequence identified by SEQ ID NO: 47, wherein the stem-loop has a deltaG (ΔG) that is at least 50%, at least 60%, at least 70%, at least 80%, at least 90%, at least 95%, at least 96%, at least 97%, at least 98%, at least 99%, or 100% of the deltaG (ΔG) of the stem-loop identified by SEQ ID NO: 47. In some embodiments, the structural RNA element comprising a stem-loop comprises a nucleotide sequence that is about 80%, about 85%, about 90%, about 95%, about 96%, about 97%, about 98%, about 99%, or 100% identical to a nucleotide sequence identified by SEQ ID NO: 47, wherein the stem-loop has a deltaG (ΔG) of about −30 kcal/mol, about −20 to −30 kcal/mol, about −20 kcal/mol, about −10 to −20 kcal/mol, about −10 kcal/mol, or about −5 to −10 kcal/mol.

In some embodiments, the structural RNA element comprising a stem-loop comprises the nucleotide sequence of SEQ ID NO: 6, wherein the structural RNA element comprising a stem-loop is located 10 nucleotides upstream of a Kozak-like sequence in the 5′ UTR.

In some embodiments, the structural RNA element comprising a stem-loop comprises the nucleotide sequence of SEQ ID NO: 6, wherein the structural RNA element comprising a stem-loop is located 45 nucleotides upstream of a Kozak-like sequence in the 5′ UTR.

In some embodiments, the structural RNA element comprising a stem-loop comprises the nucleotide sequence of SEQ ID NO: 6, wherein the structural RNA element comprising a stem-loop is located 28 nucleotides upstream of a Kozak-like sequence in the 5′ UTR.

In some embodiments, the structural RNA element comprising a stem-loop comprises the nucleotide sequence of SEQ ID NO: 6, wherein the structural RNA element comprising a stem-loop is located 41 nucleotides downstream of the 5′ cap or 5′ end of the mRNA in the 5′ UTR.

In some embodiments, the structural RNA element comprising a stem-loop comprises the nucleotide sequence of SEQ ID NO: 6, wherein the structural RNA element comprising a stem-loop is located 6 nucleotides downstream of the 5′ cap or 5′ end of the mRNA in the 5′ UTR.

In some embodiments, the structural RNA element comprising a stem-loop comprises the nucleotide sequence of SEQ ID NO: 6, wherein the structural RNA element comprising a stem-loop is located 23 nucleotides downstream of the 5′ cap or 5′ end of the mRNA in the 5′ UTR.

In some embodiments, leaky scanning of an mRNA comprising a 5′UTR comprising a structural RNA element comprising a stem-loop of the disclosure is reduced by about 1-fold, about 2-fold, about 3-fold, about 4-fold, about 5-fold, about 6-fold, about 7-fold, about 8-fold, about 9-fold, or about 10-fold relative to the leaky scanning of an mRNA comprising a 5′UTR without the structural RNA element comprising a stem-loop. In some embodiments, the leaky scanning of an mRNA comprising a structural RNA element comprising a stem-loop is reduced by about 5%, about 10%, about 15%, about 20%, about 30%, about 40%, about 50%, about 60%, about 70%, about 80%, about 90%, about 95%, about 96%, about 97%, about 98%, about 99%, or 100% relative to the leaky scanning of an mRNA without the structural RNA element comprising a stem-loop.

TABLE 1
Exemplary Structural RNA Elements
Comprising a Stem-Loop

| | | SEQ ID |
|---|---|---|
| 5′ UTRs | Sequence | NO |
| RNAse P stem | TCTCCCTGAGCTTCAGGGAG | 5 |
| loop (DNA) | | |
| RNAse P stem | UCUCCCUGAGCUUCAGGGAG | 6 |
| loop (RNA) | | |
| MRP stem loop | AGAAGCGTATCCCGCTGAGC | 7 |
| (DNA) | | |
| MRP stem loop | AGAAGCGUAUCCCGCUGAGC | 47 |
| (RNA) | | |

In some embodiments, the structural RNA element comprising a stem-loop comprises one or more nucleotide substitutions. In some embodiments, the structural RNA element comprising a stem-loop comprises one or more (e.g., 1, 2, 3 or 4) different modified nucleobases, nucleosides, or nucleotides, such as those described herein. In some embodiments, the mRNAs provided by the disclosure comprise a structural RNA element comprising a stem-loop which differs from a naturally-occurring structural RNA element comprising a stem-loop by 1, 2, 3, 4, 5, 6, 7, 8, 9, 10, 11, 12, 13, 14, 15, 16, 17, 18, 19, or 20 or more nucleotides. In some embodiments, the mRNAs provided by the disclosure comprise a structural RNA element comprising a stem-loop which differs from a naturally-occurring structural RNA element comprising a stem-loop by 1-5, 5-10, 10-15, 15-20, 20-25, 25-30, 30-35, 35-40, 40-45, 45-50 or about 50 or more nucleotides. In some embodiments, an mRNA provided by the disclosure comprises a structural RNA element comprising a stem-loop comprising a nucleotide sequence selected from the group consisting of: SEQ ID NO: 116, SEQ ID NO: 120, and SEQ ID NO: 124.

In some embodiments, an mRNA provided by the disclosure comprises a structural RNA element comprising a stem-loop comprising a nucleotide sequence at least 50%, at least 60%, at least 70%, at least 80%, at least 85%, at least 90%, at least 95%, at least 98%, at least 99%, or 100% identical to a nucleotide sequence selected from the group consisting of: SEQ ID NO: 116, SEQ ID NO: 120, and SEQ ID NO: 124.

In some embodiments, the structural RNA element comprising a stem-loop increases an expression level of a polypeptide translated from the mRNA relative to an mRNA that does not comprise the structural RNA element comprising a stem-loop. In some embodiments, the structural RNA element comprising a stem-loop increases an activity of a polypeptide translated from the mRNA relative to an mRNA that does not comprise the structural RNA element comprising a stem-loop. In some embodiments, the structural RNA element comprising a stem-loop increases an expression level and an activity of a polypeptide translated from the mRNA relative to an mRNA that does not comprise the structural RNA element comprising a stem-loop. In some embodiments, the expression level and/or activity is increased by at least about 1-fold, about 2-fold, about 3-fold, about 4-fold, about 5-fold, about 6-fold, about 7-fold, about 8-fold, about 9-fold, about 10-fold or more. In some embodiments, the expression level and/or activity is increased by about 1%, about 2%, about 3%, about 4%, about 5%, about 6%, about 7%, about 8%, about 9%, about 10%, about 15% about 20%, about 30%, about 40%, about 50%, about 60%, about 70%, about 80%, about 90%, or about 100%.

### GC-Rich Elements

In some aspects, the disclosure provides an mRNA having one or more structural modifications that inhibits leaky scanning and/or promotes the translational fidelity of mRNA translation, wherein at least one of the structural modifications is a GC-rich RNA element. In some aspects, the disclosure provides an mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA. In one embodiment, the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA. In another embodiment, the GC-rich RNA element is located 15-30, 15-20, 15-25, 10-15, or 5-10 nucleotides upstream of a Kozak consensus sequence. In another embodiment, the GC-rich RNA element is located immediately adjacent to a Kozak consensus sequence in the 5′ UTR of the mRNA.

In any of the foregoing or related aspects, the disclosure provides a GC-rich RNA element which comprises a sequence of 3-30, 5-25, 10-20, 15-20, about 20, about 15, about 12, about 10, about 7, about 6 or about 3 nucleotides, derivatives or analogs thereof, linked in any order, wherein the sequence composition is 70-80% cytosine, 60-70% cytosine, 50%-60% cytosine, 40-50% cytosine, 30-40% cytosine bases. In any of the foregoing or related aspects, the disclosure provides a GC-rich RNA element which comprises a sequence of 3-30, 5-25, 10-20, 15-20, about 20, about 15, about 12, about 10, about 7, about 6 or about 3 nucleotides, derivatives or analogs thereof, linked in any order, wherein the sequence composition is about 80% cytosine, about 70% cytosine, about 60% cytosine, about 50% cytosine, about 40% cytosine, or about 30% cytosine.

In any of the foregoing or related aspects, the disclosure provides a GC-rich RNA element which comprises a sequence of 20, 19, 18, 17, 16, 15, 14, 13, 12, 11, 10, 9, 8, 7, 6, 5, 4, or 3 nucleotides, or derivatives or analogs thereof, linked in any order, wherein the sequence composition is 70-80% cytosine, 60-70% cytosine, 50%-60% cytosine, 40-50% cytosine, or 30-40% cytosine. In any of the foregoing or related aspects, the disclosure provides a GC-rich RNA element which comprises a sequence of 20, 19, 18, 17, 16, 15, 14, 13, 12, 11, 10, 9, 8, 7, 6, 5, 4, or 3 nucleotides, or derivatives or analogs thereof, linked in any order, wherein the sequence composition is about 80% cytosine, about 70% cytosine, about 60% cytosine, about 50% cytosine, about 40% cytosine, or about 30% cytosine.

In some embodiments, the disclosure provides an mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA, wherein the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA, and wherein the GC-rich RNA element comprises a sequence of 3, 4, 5, 6, 7, 8, 9, 10, 11, 12, 13, 14, 15, 16, 17, 18, 19, or 20 nucleotides, or derivatives or analogs thereof, linked in any order, wherein the sequence composition is >50% cytosine. In some embodiments, the sequence composition is >55% cytosine, >60% cytosine, >65% cytosine, >70% cytosine, >75% cytosine, >80% cytosine, >85% cytosine, or >90% cytosine.

In other aspects, the disclosure provides an mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA, wherein the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA, and wherein the GC-rich RNA element comprises a sequence of about 3-30, 5-25, 10-20, 15-20 or about 20, about 15, about 12, about 10, about 6 or about 3 nucleotides, or derivatives or analogues thereof, wherein the sequence comprises a repeating GC-motif, wherein the repeating GC-motif is [CCG]n (SEQ ID NO: 22), wherein n=1 to 10, n=2 to 8, n=3 to 6, or n=4 to 5. In some embodiments, the sequence comprises a repeating GC-motif [CCG]n (SEQ ID NO: 22), wherein n=1, 2, 3, 4 or 5. In some embodiments, the sequence comprises a repeating GC-motif [CCG]n (SEQ ID NO: 22), wherein n=1, 2, or 3. In some embodiments, the sequence comprises a repeating GC-motif [CCG]n (SEQ ID NO: 22), wherein n=1. In some embodiments, the sequence comprises a repeating GC-motif [CCG]n (SEQ ID NO: 22), wherein n=2. In some embodiments, the sequence comprises a repeating GC-motif [CCG]n (SEQ ID NO: 22), wherein n=3. In some embodiments, the sequence comprises a repeating GC-motif [CCG]n (SEQ ID NO: 22), wherein n=4. In some embodiments, the sequence comprises a repeating GC-motif [CCG]n (SEQ ID NO: 22), wherein n=5.

In another aspect, the disclosure provides an mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA, wherein the GC-rich RNA element comprises any one of the sequences set forth in Table 2. In one embodiment, the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA. In another embodiment, the GC-rich RNA element is located about 15-30, 15-20, 15-25, 10-15, or 5-10 nucleotides upstream of a Kozak consensus sequence. In another embodiment, the GC-rich RNA element is located immediately adjacent to a Kozak consensus sequence in the 5′ UTR of the mRNA.

In other aspects, the disclosure provides an mRNA comprising at least one modification, wherein at least one modification is a GC-rich RNA element comprising the sequence V1 [CCCCGGCGCC] (SEQ ID NO: 1), or derivatives or analogs thereof, preceding a Kozak consensus sequence in the 5′ UTR of the mRNA. In some embodiments, the GC-rich element comprises the sequence V1 as set forth in Table 2 located immediately adjacent to and upstream of the Kozak consensus sequence in the 5′ UTR of the mRNA. In some embodiments, the GC-rich element comprises the sequence V1 as set forth in Table 2 located 1, 2, 3, 4, 5, 6, 7, 8, 9 or 10 bases upstream of the Kozak consensus sequence in the 5′ UTR of the mRNA. In other embodiments, the GC-rich element comprises the sequence V1 as set forth in Table 2 located 1-3, 3-5, 5-7, 7-9, 9-12, or 12-15 bases upstream of the Kozak consensus sequence in the 5′ UTR of the mRNA.

In other aspects, the disclosure provides an mRNA comprising at least one modification, wherein at least one modification is a GC-rich RNA element comprising the sequence V2 [CCCCGGC](SEQ ID NO: 2), or derivatives or analogs thereof, preceding a Kozak consensus sequence in the 5′ UTR of the mRNA. In some embodiments, the GC-rich element comprises the sequence V2 as set forth in Table 2 located immediately adjacent to and upstream of the Kozak consensus sequence in the 5′ UTR of the mRNA. In some embodiments, the GC-rich element comprises the sequence V2 as set forth in Table 2 located 1, 2, 3, 4, 5, 6, 7, 8, 9 or 10 bases upstream of the Kozak consensus sequence in the 5′ UTR of the mRNA. In other embodiments, the GC-rich element comprises the sequence V2 as set forth in Table 2 located 1-3, 3-5, 5-7, 7-9, 9-12, or 12-15 bases upstream of the Kozak consensus sequence in the 5′ UTR of the mRNA.

In other aspects, the disclosure provides an mRNA comprising at least one modification, wherein at least one modification is a GC-rich RNA element comprising the sequence EK2 [GCCGCC](SEQ ID NO: 18), or derivatives or analogs thereof, preceding a Kozak consensus sequence in the 5′ UTR of the mRNA. In some embodiments, the GC-rich element comprises the sequence EK2 as set forth in Table 2 located immediately adjacent to and upstream of the Kozak consensus sequence in the 5′ UTR of the mRNA. In some embodiments, the GC-rich element comprises the sequence EK2 as set forth in Table 2 located 1, 2, 3, 4, 5, 6, 7, 8, 9 or 10 bases upstream of the Kozak consensus sequence in the 5′ UTR of the mRNA. In other embodiments, the GC-rich element comprises the sequence EK2 as set forth in Table 2 located 1-3, 3-5, 5-7, 7-9, 9-12, or 12-15 bases upstream of the Kozak consensus sequence in the 5′ UTR of the mRNA.

In yet other aspects, the disclosure provides an mRNA comprising at least one modification, wherein at least one modification is a GC-rich RNA element comprising the sequence V1 [CCCCGGCGCC] (SEQ ID NO: 1), or derivatives or analogs thereof, preceding a Kozak consensus sequence in the 5′ UTR of the mRNA, wherein the 5′ UTR comprises the following sequence:

GGGAAAUAAGAGAGAAAAGAAGAGUAAGAAGAAAUAUAAGA (SEQ ID NO: 56).

In some embodiments, the GC-rich element comprises the sequence V1 as set forth in Table 2 located immediately adjacent to and upstream of the Kozak consensus sequence in the 5′ UTR sequence shown in Table 2 (SEQ ID NOs: 17 or 48). In some embodiments, the GC-rich element comprises the sequence V1 as set forth in Table 2 located 1, 2, 3, 4, 5, 6, 7, 8, 9 or 10 bases upstream of the Kozak consensus sequence in the 5′ UTR of the mRNA, wherein the 5′ UTR comprises the following sequence:

GGGAAAUAAGAGAGAAAAGAAGAGUAAGAAGAAAUAUAAGA (SEQ ID NO: 56)

In other embodiments, the GC-rich element comprises the sequence V1 as set forth in Table 2 located 1-3, 3-5, 5-7, 7-9, 9-12, or 12-15 bases upstream of the Kozak consensus sequence in the 5′ UTR of the mRNA, wherein the 5′ UTR comprises the following sequence:

(SEQ ID NO: 56)
GGGAAAUAAGAGAGAAAAGAAGAGUAAGAAGAAAUAUAAGA.

In some embodiments, the 5′ UTR comprises the following sequence:

(5′v1.1, SEQ ID NO: 4)
GGGAAAUAAGAGAGAAAAGAAGAGUAAGAAGAAAUAUAAGA
CCCCGGCGCCGCCACC

In another aspect, the disclosure provides an mRNA comprising at least one modification, wherein at least one modification is a GC-rich RNA element comprising a stable RNA secondary structure comprising a sequence of nucleotides, or derivatives or analogs thereof, linked in an order which forms a hairpin or a stem-loop. In one embodiment, the stable RNA secondary structure is upstream or downstream of the initiation codon. In another embodiment, the stable RNA secondary structure is located about 30, about 25, about 20, about 15, about 10, or about 5 nucleotides upstream or downstream of the initiation codon. In another embodiment, the stable RNA secondary structure is located about 20, about 15, about 10 or about 5 nucleotides upstream or downstream of the initiation codon. In another embodiment, the stable RNA secondary structure is located about 5, about 4, about 3, about 2, about 1 nucleotides upstream or downstream of the initiation codon. In another embodiment, the stable RNA secondary structure is located about 15-30, about 15-20, about 15-25, about 10-15, or about 5-10 nucleotides upstream or downstream of the initiation codon. In another embodiment, the stable RNA secondary structure is located 12-15 nucleotides upstream and downstream of the initiation codon. In another embodiment, the stable RNA secondary structure comprises the initiation codon. In another embodiment, the stable RNA secondary structure has a deltaG of about −30 kcal/mol, about −20 to −30 kcal/mol, about −20 kcal/mol, about −10 to −20 kcal/mol, about −10 kcal/mol, about −5 to −10 kcal/mol.

In another embodiment, the modification is operably linked to an open reading frame encoding a polypeptide and wherein the modification and the open reading frame are heterologous.

In another embodiment, the sequence of the GC-rich RNA element is comprised exclusively of guanine (G) and cytosine (C) nucleobases.

Exemplary GC-rich RNA elements useful in the mRNAs provided by the disclosure are provided in Table 2.

TABLE 2
Exemplary GC-Rich RNA Elements

| | | SEQ |
|---|---|---|
| | | ID |
| | Sequence | NO |
| GC-Rich RNA Elements | | |
| K0 (Traditional | [GCCACC] | 17 |
| Kozak consensus) | | |
| Kozak-like sequence | [GCCGCC] | 48 |
| EK1 | [CCCGCC] | 3 |
| EK2 | [GCCGCC] | 18 |
| EK3 | [CCGCCG] | 19 |
| V1 | [CCCCGGCGCC] | 1 |
| V2 | [CCCCGGC] | 2 |
| CG1 | [GCGCCCCGCGGCGCCCCGCG] | 20 |
| CG2 | [CCCGCCCGCCCCGCCCCGCC] | 21 |
| (CCG)ₙ n= 1-10 | [CCG]ₙ | 22 |
| (GCC)ₙ, n = 1-10 | [GCC]ₙ | 23 |
| Stable RNA Secondary | | |
| Structures | | |
| SL1 | CCGCGGCGCCCCGCGG | 24 |
| | (−9.90 kcal/mol) | |
| SL2 | GCGCGCAUAUAGCGCGC | 25 |
| | (−10.90 kcal/mol) | |
| SL3 | CAUGGUGGCGGCCCGCCGCCACC | 49 |
| | AUG (−22.10 kcal/mol) | |
| SL4 | CAUGGUGGCCCGCCGCCACCAUG | 50 |
| | (−14.90 kcal/mol) | |
| SL5 | CAUGGUGCCCGCCGCCACCAUG | 51 |
| | (−8.00 kcal/mol) | |

### C-Rich Elements

In some aspects, the disclosure provides an mRNA having one or more structural modifications that inhibit leaky scanning and/or promote the translational fidelity of mRNA translation, wherein at least one of the structural modifications is a C-rich RNA element. In some aspects, the disclosure provides an mRNA comprising at least one modification, wherein at least one modification is a C-rich RNA element comprising a sequence of linked nucleotides, or derivatives or analogs thereof, located proximal to the 5′ cap or 5′ end of the mRNA, wherein the C-rich element comprises a sequence of linked nucleotides, or derivatives or analogs thereof, in a 5′ UTR of the mRNA. In one embodiment, the C-rich RNA element is located about 45-50, about 40-45, about 35-40, about 30-35 about 25-30, about 20-25, about 15-20, about 10-15, about 6-10, about 1-5 nucleotides, or about 20, 19, 18, 17, 16, 15, 14, 13, 12, 11, 10, 9, 8, 7, 6, 5, 4, 3, 2 or 1 nucleotide(s) downstream of the 5′ cap or 5′ end of the mRNA. In some embodiments, the C-rich element is located about 1-20, about 2-15, about 3-10, about 4-8 or about 6 nucleotides downstream of the 5′ cap or 5′ end of the mRNA. In some embodiments, the C-rich element is located downstream of the 5′ cap or 5′ end of the mRNA with a transcription start site located between the 5′ cap or 5′end of the mRNA and the C-rich element

In some embodiments, the C-rich RNA element comprises a sequence of about 100%, about 95%, about 90%, about 85%, about 80%, about 75%, about 70%, about 65%, about 60%, about 55%, about 50%, or greater than 50% cytosine nucleobases or derivatives or analogs thereof. In some embodiments, the C-rich RNA element comprises a sequence of less than about 25%, less than about 20%, less than about 15%, less than about 10%, or less than about 5% guanosine nucleobases, or derivatives or analogs thereof. In some embodiments, the C-rich RNA element comprises a sequence of less than about 50%, less than about 45%, less than about 40%, less than about 35%, less than about 30%, less than about 25%, less than about 20%, less than about 15%, less than about 10%, or less than about 5% guanosine nucleobases, or derivatives or analogs thereof. In some embodiments, the C-rich RNA element comprises a sequence of less than about 25% guanosine nucleobases, or derivatives or analogs thereof.

In some embodiments, the C-rich RNA element is located upstream of a Kozak-like sequence in the 5′UTR. In some embodiments, the C-rich RNA element is located about 50, about 45, about 40, about 35, about 30, about 25, about 20, about 15, about 10 or about 5 nucleotides upstream of a Kozak-like sequence in the 5′UTR. In some embodiments, the C-rich RNA element is located about 5, about 4, about 3, about 2 or about 1 nucleotide upstream of a Kozak-like sequence in the 5′UTR. In some embodiments, the C-rich RNA element is located about 15-50, about 15-40, about 15-30, about 15-20, about 10-15 or about 5-10 nucleotides upstream of a Kozak-like sequence in the 5′UTR. In some embodiments, the C-rich RNA element is located upstream of and immediately adjacent to a Kozak-like sequence in the 5′UTR.

In some embodiments, the C-rich RNA element comprises a sequence of about 3-20, about 4-18, about 6-16, about 6-14, about 6-12, about 6-10, about 8-14, about 8-12, about 8-10, about 10-12, about 10-14, about 14, about 12, about 11, about 10 or about 20, 19, 18, 17, 16, 15, 14, 13, 12, 11, 10, 9, 8, 7, 6, 5, 4 or 3 nucleotides, derivatives or analogs thereof, linked in any order. In some embodiments, the C-rich RNA element comprises a sequence of about 20 nucleotides. In some embodiments, the C-rich RNA element comprises a sequence of about 19 nucleotides. In some embodiments, the C-rich RNA element comprises a sequence of about 18 nucleotides. In some embodiments, the C-rich RNA element comprises a sequence of about 17 nucleotides. In some embodiments, the C-rich RNA element comprises a sequence of about 16 nucleotides. In some embodiments, the C-rich RNA element comprises a sequence of about 15 nucleotides. In some embodiments, the C-rich RNA element comprises a sequence of about 14 nucleotides. In some embodiments, the C-rich RNA element comprises a sequence of about 13 nucleotides. In some embodiments, the C-rich RNA element comprises a sequence of about 12 nucleotides. In some embodiments, the C-rich RNA element comprises a sequence of about 11 nucleotides. In some embodiments, the C-rich RNA element comprises a sequence of about 10 nucleotides. In some embodiments, the C-rich RNA element comprises a sequence of about 9 nucleotides. In some embodiments, the C-rich RNA element comprises a sequence of about 8 nucleotides. In some embodiments, the C-rich RNA element comprises a sequence of about 7 nucleotides. In some embodiments, the C-rich RNA element comprises a sequence of about 6 nucleotides. In some embodiments, the C-rich RNA element comprises a sequence of about 5 nucleotides. In some embodiments, the C-rich RNA element comprises a sequence of about 4 nucleotides. In some embodiments, the C-rich RNA element comprises a sequence of about 3 nucleotides.

In some embodiments, the C-rich RNA element comprises a sequence of about 3-20, about 4-18, about 6-16, about 6-14, about 6-12, about 6-10, about 8-14, about 8-12, about 8-10, about 10-12, about 10-14, about 14, about 12, about 11, about 10 or about 20, 19, 18, 17, 16, 15, 14, 13, 12, 11, 10, 9, 8, 7, 6, 5, 4 or 3 nucleotides, derivatives or analogs thereof, linked in any order, wherein the sequence composition is about 100%, about 95%, about 90%, about 85%, about 80%, about 75%, about 70%, about 65%, about 60%, about 55% or about 50% cytosine bases. In some embodiments, the C-rich RNA element comprises a sequence of about 14 nucleotides, derivatives or analogs thereof, linked in any order, wherein the sequence composition is about 100%, about 95%, about 90%, about 85%, about 80%, about 75%, about 70%, about 65%, about 60%, about 55% or about 50% cytosine bases. In some embodiments, the C-rich RNA element comprises a sequence of about 14 nucleotides, derivatives or analogs thereof, linked in any order, wherein the sequence composition is greater than about 90% cytosine bases. In some embodiments, the C-rich RNA element comprises a sequence of about 13 nucleotides, derivatives or analogs thereof, linked in any order, wherein the sequence composition is about 100%, about 95%, about 90%, about 85%, about 80%, about 75%, about 70%, about 65%, about 60%, about 55% or about 50% cytosine bases. In some embodiments, the C-rich RNA element comprises a sequence of about 13 nucleotides, derivatives or analogs thereof, linked in any order, wherein the sequence composition is greater than about 90% cytosine bases. In some embodiments, the C-rich RNA element comprises a sequence of about 12 nucleotides, derivatives or analogs thereof, linked in any order, wherein the sequence composition is about 100%, about 95%, about 90%, about 85%, about 80%, about 75%, about 70%, about 65%, about 60%, about 55% or about 50% cytosine bases. In some embodiments, the C-rich RNA element comprises a sequence of about 12 nucleotides, derivatives or analogs thereof, linked in any order, wherein the sequence composition is greater than about 90% cytosine bases. In some embodiments, the C-rich RNA element comprises a sequence of about 11 nucleotides, derivatives or analogs thereof, linked in any order, wherein the sequence composition is about 100%, about 95%, about 90%, about 85%, about 80%, about 75%, about 70%, about 65%, about 60%, about 55% or about 50% cytosine bases. In some embodiments, the C-rich RNA element comprises a sequence of about 11 nucleotides, derivatives or analogs thereof, linked in any order, wherein the sequence composition is greater than about 90% cytosine bases. In some embodiments, the C-rich RNA element comprises a sequence of about 10 nucleotides, derivatives or analogs thereof, linked in any order, wherein the sequence composition is about 100%, about 95%, about 90%, about 85%, about 80%, about 75%, about 70%, about 65%, about 60%, about 55% or about 50% cytosine bases. In some embodiments, the C-rich RNA element comprises a sequence of about 10 nucleotides, derivatives or analogs thereof, linked in any order, wherein the sequence composition is greater than about 90% cytosine bases.

In some embodiments, the C-rich RNA element is depleted of guanosine. In some embodiments, the C-rich element comprises a sequence of less than about 25%, less than about 20%, less than about 15%, less than about 10% or less than about 5% guanosine bases.

In some embodiments, the C-rich RNA element comprises a sequence of about 14 nucleotides, derivatives or analogs thereof, linked in any order, wherein the sequence composition is about 100%, about 95%, about 90%, about 85%, about 80%, about 75%, about 70%, about 65%, about 60%, about 55% or about 50% cytosine bases, wherein the sequence is located upstream of a Kozak-like sequence in the 5′UTR, and wherein the sequence is located downstream of the 5′cap or 5′end of the mRNA. In some embodiments, the C-rich RNA element comprises a sequence of about 13 nucleotides, derivatives or analogs thereof, linked in any order, wherein the sequence composition is about 100%, about 95%, about 90%, about 85%, about 80%, about 75%, about 70%, about 65%, about 60%, about 55% or about 50% cytosine bases, wherein the sequence is located upstream of a Kozak-like sequence in the 5′UTR, and wherein the sequence is located downstream of the 5′cap or 5′end of the mRNA. In some embodiments, the C-rich RNA element comprises a sequence of about 12 nucleotides, derivatives or analogs thereof, linked in any order, wherein the sequence composition is about 100%, about 95%, about 90%, about 85%, about 80%, about 75%, about 70%, about 65%, about 60%, about 55% or about 50% cytosine bases, wherein the sequence is located upstream of a Kozak-like sequence in the 5′UTR, and wherein the sequence is located downstream of the 5′cap or 5′end of the mRNA. In some embodiments, the C-rich RNA element comprises a sequence of about 11 nucleotides, derivatives or analogs thereof, linked in any order, wherein the sequence composition is about 100%, about 95%, about 90%, about 85%, about 80%, about 75%, about 70%, about 65%, about 60%, about 55% or about 50% cytosine bases, wherein the sequence is located upstream of a Kozak-like sequence in the 5′UTR, and wherein the sequence is located downstream of the 5′cap or 5′end of the mRNA. In some embodiments, the C-rich RNA element comprises a sequence of about 10 nucleotides, derivatives or analogs thereof, linked in any order, wherein the sequence composition is about 100%, about 95%, about 90%, about 85%, about 80%, about 75%, about 70%, about 65%, about 60%, about 55% or about 50% cytosine bases, wherein the sequence is located upstream of a Kozak-like sequence in the 5′UTR, and wherein the sequence is located downstream of the 5′cap or 5′end of the mRNA.

In some embodiments, the C-rich RNA element comprises a sequence comprising the formula 5′-[C1]ᵥ-[N1]_{w}-[N2]ₓ-[N3]_{y}-[C2]_{z}-3′, wherein C1 and C2 are nucleotides comprising cytidine, or a derivative or analogue thereof, wherein N1, and N2 and N3 if present, are each a nucleotide comprising a nucleobase selected from the group consisting of: adenine, guanine, thymine, uracil, and cytosine, and derivatives or analogues thereof (e.g., pseudouridine, N1-methyl pseudouridine, 5-methoxyuridine), wherein v, w, x, y and z are integers whose value indicates the number of nucleotides comprising the C-rich RNA element.

In some embodiments, v=12-15 nucleotides, 3-12 nucleotides, 5-10 nucleotides, 6-8 nucleotides, 3, 4, 5, 6, 7, 8, 9 or 10 nucleotides. In some embodiments, z=2-10 nucleotides, 2-7 nucleotides, 3-5 nucleotides, 2, 3, 4, 5, 6, or 7 nucleotides. In some embodiments, w-1-5 nucleotides, 1-3 nucleotides, 1, 2, or 3 nucleotide(s). In some embodiments, x=0-5 nucleotides, 0-3 nucleotides, 0, 1, 2, or 3 nucleotide(s). In some embodiments, y=0-5 nucleotides, 0-3 nucleotides, 0, 1, 2, or 3 nucleotide(s).

In some embodiments, N1 comprises adenosine, or derivative or analogue thereof; w=1 or 2; x=0, 1, 2, or 3; and y=0, 1, 2, or 3. In some embodiments, N1 comprises adenosine, or derivative or analogue thereof; w=1 or 2; x=0; and y=0. In some embodiments, N1 comprises uracil, or derivative or analogue thereof (e.g., pseudouridine, N1-methyl pseudouridine, 5-methoxyuridine); w=1 or 2; N2 comprises adenosine, or derivative or analogue thereof; x=1, 2, or 3; N3 is guanosine, or derivative or analogue thereof; and y=1 or 2. In some embodiments, N1 comprises uracil, or derivative or analogue thereof (e.g., pseudouridine, N1-methyl pseudouridine, 5-methoxyuridine); w=1; N2 comprises adenosine, or derivative or analogue thereof; x=2; N3 is guanosine, or derivative or analogue thereof; and y=1.

In some embodiments, the C-rich RNA element comprises the formula

5′-[C1]ᵥ-[N1]_{w}-[N2]ₓ-[N3]_{y},-[C2]_{z}-3′,

wherein C1 and C2 are nucleotides comprising cytidine, or a derivative or analogue thereof, wherein N1, and N2 and N3 if present, are each a nucleotide comprising a nucleobase selected from the group consisting of: adenine, guanine, and uracil, and derivatives or analogues thereof, (e.g., pseudouridine, N1-methyl pseudouridine, 5-methoxyuridine), wherein v, w, x, y and z are integers whose value indicates the number of nucleotides comprising the C-rich RNA element. In some embodiments, v=4-10 nucleotides, 6-8 nucleotides, 6, 7, or 8 nucleotides. In some embodiments, w=1-3 nucleotides, 1 or 2 nucleotide(s). In some embodiments, x=0-3 nucleotides, 0, 1 or 2 nucleotide(s). In some embodiments, y=0-3 nucleotides, 0 or 1 nucleotide(s). In some embodiments, z=2-6 nucleotides, 2-5 nucleotides, 2, 3, 4, or 5 nucleotides. In some embodiments, N1 comprises adenosine, or derivative or analogue thereof; w=1; x=0; and y=0. In some embodiments, N1 comprises adenosine, or derivative or analogue thereof; w=2; x=0; and y=0. In some embodiments, N1 comprises uracil, or derivative or analogue thereof (e.g., pseudouridine, N1-methyl pseudouridine, 5-methoxyuridine); w=1 or 2; N2 comprises adenosine, or derivative or analogue thereof; x=1, 2, or 3; N3 is guanosine, or derivative or analogue thereof; and y=1 or 2. In some embodiments, N1 comprises uracil, or derivative or analogue thereof (e.g., pseudouridine, N1-methyl pseudouridine, 5-methoxyuridine); w=1; N2 comprises adenosine, or derivative or analogue thereof; x=2; N3 is guanosine, or derivative or analogue thereof; and y=1.

In some embodiments, the C-rich RNA element comprises a nucleotide sequence selected from the group consisting of: SEQ ID NO: 29, SEQ ID NO: 30, SEQ ID NO: 31, SEQ ID NO: 32, SEQ ID NO: 33 and SEQ ID NO: 34. In some embodiments, the C-rich RNA element comprises the nucleotide sequence 5′-CCCCCCCAACCC-3′ (SEQ ID NO: 29). In some embodiments, the C-rich RNA element comprises the nucleotide sequence 5′-CCCCCCCCAACC-3′ (SEQ ID NO: 30). In some embodiments, the C-rich RNA element comprises the nucleotide sequence 5′-CCCCCCACCCCC-3′ (SEQ ID NO: 31). In some embodiments, the C-rich RNA element comprises the nucleotide sequence 5′-CCCCCCUAAGCC-3′ (SEQ ID NO: 32). In some embodiments, the C-rich RNA element comprises the nucleotide sequence 5′-CCCCACAACC-3′ (SEQ ID NO: 33). In some embodiments, the C-rich RNA element comprises the nucleotide sequence 5′-CCCCCACAACC-3′ (SEQ ID NO: 34)

Exemplary C-rich elements provided by the disclosure are set forth in Table 3. These C-rich elements and 5′UTR are useful in the mRNAs of the disclosure.

TABLE 3
C-Rich RNA Elements

| C-Rich RNA Element | Sequence | SEQ ID NO |
|---|---|---|
| CR1 | CCCCCCCCAACC | 30 |
| CR2 | CCCCCCCAACCC | 29 |
| CR3 | CCCCCCACCCCC | 31 |
| CR4 | CCCCCCUAAGCC | 32 |
| CR5 | CCCCACAACC | 33 |
| CR6 | CCCCCACAACC | 34 |

### Combination of RNA Elements

In some aspects, the disclosure provides an mRNA comprising a 5′UTR comprising both a C-rich RNA element and a GC-rich RNA element, such as those described herein. In some embodiments, the amount or extent of leaky scanning from the mRNA is additively or synergistically decreased by a combination of a C-rich RNA element and the GC-rich RNA element of the disclosure. In some embodiments, leaky scanning of an mRNA comprising a 5′UTR comprising a C-rich RNA element and a GC-rich RNA element of the disclosure is reduced by about 1-fold, about 2-fold, about 3-fold, about 4-fold, about 5-fold, about 6-fold, about 7-fold, about 8-fold, about 9-fold, about 10-fold relative to the leaky scanning of an mRNA comprising a 5′UTR comprising a C-rich RNA element alone or an mRNA comprising a 5′UTR comprising a GC-rich RNA element alone. In some embodiments, leaky scanning of an mRNA comprising a 5′UTR comprising a C-rich RNA element and a GC-rich RNA element of the disclosure is reduced by about 1-fold, about 2-fold, about 3-fold, about 4-fold, about 5-fold, about 6-fold, about 7-fold, about 8-fold, about 9-fold, about 10-fold relative to the leaky scanning of an mRNA comprising a 5′UTR without a C-rich RNA element or a GC-rich RNA element. In some embodiments, the leaky scanning of an mRNA comprising a 5′UTR comprising a C-rich RNA element and a GC-rich RNA element is reduced by about 5%, about 10%, about 15%, about 20%, about 30%, about 40%, about 50%, about 60%, about 70%, about 80%, about 90% or about 100% relative to the leaky scanning of an mRNA comprising a 5′UTR comprising a C-rich RNA element alone or an mRNA comprising a 5′UTR comprising a GC-rich RNA element alone. In some embodiments, the leaky scanning of an mRNA comprising a 5′UTR comprising a C-rich RNA element and a GC-rich RNA element is reduced by about 5%, about 10%, about 15%, about 20%, about 30%, about 40%, about 50%, about 60%, about 70%, about 80%, about 90% or about 100% relative to the leaky scanning of an mRNA comprising a 5′UTR comprising without a C-rich RNA element or a GC-rich RNA element. In some embodiments, the leaky scanning of an mRNA comprising a C-rich RNA element and a GC-rich RNA element is abolished or undetectable.

In some aspects, the disclosure provides an mRNA comprising one or more C-rich RNA elements (e.g., 2, 3, 4) and one or more GC-rich RNA elements (e.g., 2, 3, 4).

In some embodiments, the disclosure provides an mRNA having a GC-rich RNA element and a C-rich RNA element as described herein, wherein the C-rich RNA element and the GC-rich RNA element precede a Kozak-like sequence or Kozak consensus sequence, in the 5′ UTR. In some embodiments, the C-rich RNA element is upstream the GC-rich RNA element in the 5′UTR. In some embodiments, the C-rich RNA element is proximal to the 5′ end or 5′ cap of the mRNA relative to the location of the GC-rich RNA element in the 5′ UTR. In some embodiments, the C-rich RNA element is located adjacent to or within about 1-6, or about 1-10 nucleotides of the 5′end or 5′ cap of the mRNA and the GC-rich RNA element is located proximal to the Kozak-like sequence or Kozak consensus sequence in the 5′ UTR. In some embodiments, the C-rich RNA element is located adjacent to or within about 1-6, or about 1-10 nucleotides of the 5′end or 5′ cap of the mRNA and the GC-rich RNA element is located adjacent to or within about 1-6 or about 1-10 nucleotides of the Kozak-like sequence or Kozak consensus sequence in the 5′ UTR.

In some embodiments, a 5′ UTR comprising both a GC-rich RNA element and a C-rich RNA element provides enhanced translational regulatory activity compared to a 5′UTR comprising a GC-rich RNA element or a C-rich RNA element.

In some aspects, the disclosure provides an mRNA, wherein the mRNA comprises: a 5′ cap, a 5′ untranslated region (UTR), a Kozak-like sequence, an initiation codon, a full open reading frame encoding a polypeptide, and a 3′ UTR, wherein the 5′ UTR comprises a C-rich RNA element comprising a nucleotide sequence selected from the group consisting of: SEQ ID NO: 29, SEQ ID NO: 30, SEQ ID NO: 31, SEQ ID NO: 32, SEQ ID NO: 33 and SEQ ID NO: 34, and comprises a GC-rich RNA element comprising a nucleotide sequence selected from the group consisting of: SEQ ID NO: 1, SEQ ID NO: 2, SEQ ID NO: 3, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 49, SEQ ID NO: 50 and SEQ ID NO: 51.

In some embodiments, the C-rich RNA element comprises a nucleotide sequence selected from the group consisting of SEQ ID NO: 31, SEQ ID NO: 32 and SEQ ID NO: 33, and the GC-rich RNA element comprises a nucleotide sequence selected from the group consisting of: SEQ ID NO: 1, SEQ ID NO: 2 and SEQ ID NO: 23.

In some aspects, the disclosure provides an mRNA, wherein the mRNA comprises: a 5′ cap, a 5′ untranslated region (UTR), a Kozak-like sequence, an initiation codon, a full open reading frame encoding a polypeptide, and a 3′ UTR, wherein the 5′ UTR comprises a C-rich RNA element comprising the nucleotide sequence set forth in SEQ ID NO: 31 and a GC-rich RNA element comprising the nucleotide sequence set forth in SEQ ID NO: 1.

In some aspects, the disclosure provides an mRNA, wherein the mRNA comprises: a 5′ cap, a 5′ untranslated region (UTR), a Kozak-like sequence, an initiation codon, a full open reading frame encoding a polypeptide, and a 3′ UTR, wherein the 5′ UTR comprises a C-rich RNA element comprising the nucleotide sequence set forth in SEQ ID NO: 33 and a GC-rich RNA element comprises the nucleotide sequence set forth in SEQ ID NO: 1.

In some aspects, the disclosure provides an mRNA, wherein the mRNA comprises: a 5′ cap, a 5′ untranslated region (UTR), a Kozak-like sequence, an initiation codon, a full open reading frame encoding a polypeptide, and a 3′ UTR, wherein the 5′ UTR comprises a C-rich RNA element comprising the nucleotide sequence set forth in SEQ ID NO: 32 and a GC-rich RNA element comprises the nucleotide sequence set forth in SEQ ID NO: 23.

In some aspects, the disclosure provides an mRNA, wherein the mRNA comprises: a 5′ cap, a 5′ untranslated region (UTR), a Kozak-like sequence, an initiation codon, a full open reading frame encoding a polypeptide, and a 3′ UTR, wherein the 5′ UTR comprises a C-rich RNA element comprising the nucleotide sequence set forth in SEQ ID NO: 32 and a GC-rich RNA element comprises the nucleotide sequence [GCC]n set forth in SEQ ID NO: 23, where n=3.

In some aspects, the disclosure provides an mRNA, wherein the mRNA comprises a 5′ UTR comprising a C-rich RNA element and a GC-rich RNA element, wherein the 5′UTR comprises the nucleotide sequence set forth in SEQ ID NO: 35.

In some aspects, the disclosure provides an mRNA, wherein the mRNA comprises a 5′ UTR comprising a C-rich RNA element and a GC-rich RNA element, wherein the 5′UTR comprises the nucleotide sequence set forth in SEQ ID NO: 36.

In some aspects, the disclosure provides an mRNA, wherein the mRNA comprises a 5′ UTR comprising a C-rich RNA element and a GC-rich RNA element, wherein the 5′UTR comprises the nucleotide sequence set forth in SEQ ID NO: 40.

In some aspects, the disclosure provides an mRNA, wherein the mRNA comprises a 5′ UTR comprising a C-rich RNA element and a GC-rich RNA element, wherein the 5′UTR comprises the nucleotide sequence set forth in SEQ ID NO: 41.

In some aspects, the disclosure provides an mRNA, wherein the mRNA comprises a 5′ UTR comprising a C-rich RNA element and a GC-rich RNA element, wherein the 5′UTR comprises the nucleotide sequence set forth in SEQ ID NO: 44.

### GC-Rich RNA Element+Structural RNA Element

In some aspects, the disclosure provides an mRNA comprising a 5′ UTR comprising at least one or more GC-rich RNA element(s) described herein and at least one or more structural RNA element(s) comprising a stem-loop as described herein. In some embodiments, a 5′ UTR comprising at least one or more GC-rich RNA element(s) and at least one or more structural RNA element(s) described herein provides an enhanced translational regulatory activity compared to a 5′ UTR comprising only the at least one or more GC-rich RNA element(s) or only the at least one or more structural RNA element(s). In some embodiments, the amount or extent of leaky scanning of an mRNA comprising a 5′ UTR comprising at least one or more GC-rich RNA element(s) and at least one or more structural RNA element(s) of the disclosure is additively or synergistically reduced or decreased.

In some embodiments, leaky scanning of an mRNA comprising a 5′UTR comprising a GC-rich RNA element and a structural RNA element of the disclosure is reduced by about 1-fold, about 2-fold, about 3-fold, about 4-fold, about 5-fold, about 6-fold, about 7-fold, about 8-fold, about 9-fold, or about 10-fold relative to the leaky scanning of an mRNA comprising a 5′UTR comprising a GC-rich RNA element alone or an mRNA comprising a 5′UTR comprising a structural RNA element alone. In some embodiments, leaky scanning of an mRNA comprising a 5′UTR comprising a GC-rich RNA element and a structural RNA element of the disclosure is reduced by about 1-fold, about 2-fold, about 3-fold, about 4-fold, about 5-fold, about 6-fold, about 7-fold, about 8-fold, about 9-fold, or about 10-fold relative to the leaky scanning of an mRNA comprising a 5′UTR without a GC-rich RNA element or a structural RNA element. In some embodiments, the leaky scanning of an mRNA comprising a 5′UTR comprising a GC-rich RNA element and a structural RNA element is reduced by about 5%, about 10%, about 15%, about 20%, about 30%, about 40%, about 50%, about 60%, about 70%, about 80%, about 90%, or about 100% relative to the leaky scanning of an mRNA comprising a 5′UTR comprising a GC-rich RNA element alone or an mRNA comprising a 5′UTR comprising a structural RNA element alone. In some embodiments, the leaky scanning of an mRNA comprising a 5′UTR comprising a GC-rich RNA element and a structural RNA element is reduced by about 5%, about 10%, about 15%, about 20%, about 30%, about 40%, about 50%, about 60%, about 70%, about 80%, about 90%, or about 100% relative to the leaky scanning of an mRNA comprising a 5′UTR comprising without a GC-rich RNA element or a structural RNA element. In some embodiments, the leaky scanning of an mRNA comprising a GC-rich RNA element and a structural RNA element is abolished or undetectable.

In some aspects, the disclosure provides an mRNA comprising one or more GC-rich RNA elements (e.g., 2, 3, 4) and one or more structural RNA elements (e.g., 2, 3, 4).

In some embodiments, the disclosure provides an mRNA having a GC-rich RNA element and a structural RNA element as described herein, wherein the GC-rich RNA element and the structural RNA element precede a Kozak-like sequence or Kozak consensus sequence in the 5′ UTR. In some embodiments, the disclosure provides an mRNA having a GC-rich RNA element and a structural RNA element as described herein, wherein the GC-rich RNA element and the structural RNA element are located upstream of a Kozak-like sequence or Kozak consensus sequence in the 5′ UTR.

In some embodiments, the GC-rich RNA element is upstream of the structural RNA element in the 5′UTR. In some embodiments, the GC-rich RNA element is downstream of the structural RNA element in the 5′UTR. In some embodiments, the GC-rich RNA element is proximal to the 5′ end or 5′ cap of the mRNA relative to the location of the structural RNA element in the 5′ UTR. In some embodiments, the GC-rich RNA element is proximal to a Kozak-like sequence or Kozak consensus sequence relative to the location of the structural RNA element in the 5′ UTR.

In some embodiments, the GC-rich RNA element is located upstream and adjacent to or upstream and within about 1-6, or about 1-10 nucleotides of a Kozak-like sequence or Kozak consensus sequence of the mRNA and the structural RNA element is located upstream of the GC-rich RNA element in the 5′ UTR. In some embodiments, the GC-rich RNA element is located upstream and adjacent to or upstream and within about 1-6, or about 1-10 nucleotides of a Kozak-like sequence or Kozak consensus sequence of the mRNA and the structural RNA element is located upstream and adjacent to the GC-rich RNA element in the 5′ UTR. In some embodiments, the GC-rich RNA element is located upstream and adjacent to a Kozak-like sequence or Kozak consensus sequence of the mRNA and the structural RNA element comprising a stem-loop is located upstream and adjacent to the GC-rich RNA element in the 5′ UTR.

In some embodiments, the GC-rich RNA element is located upstream and adjacent to or within about 1-6, or about 1-10 nucleotides of a Kozak-like sequence or Kozak consensus sequence of the mRNA and the structural RNA element comprising a stem loop is located about 45-50, about 40-45, about 35-40, about 30-35, about 25-30, about 20-25, about 15-20, about 10-15, about 6-10 nucleotides, about 1-5 nucleotides, or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) upstream of the GC-rich RNA element in the 5′ UTR.

In some embodiments, the GC-rich RNA element is located upstream and adjacent to or within about 1-6, or about 1-10 nucleotides of a Kozak-like sequence or Kozak consensus sequence of the mRNA and the structural RNA element comprising a stem loop is located 35 nucleotides upstream of the GC-rich RNA element. In some embodiments, the GC-rich RNA element is located upstream and adjacent to a Kozak-like sequence or Kozak consensus sequence of the mRNA and the structural RNA element comprising a stem loop is located 35 nucleotides upstream of the GC-rich RNA element.

In some embodiments, the GC-rich RNA element is located upstream and adjacent to or within about 1-6, or about 1-10 nucleotides of a Kozak-like sequence or Kozak consensus sequence of the mRNA and the structural RNA element comprising a stem loop is located 18 nucleotides upstream of the GC-rich RNA element. In some embodiments, the GC-rich RNA element is located upstream and adjacent to a Kozak-like sequence or Kozak consensus sequence of the mRNA and the structural RNA element comprising a stem loop is located 18 nucleotides upstream of the GC-rich RNA element.

In some embodiments, the structural RNA element comprising a stem loop is located about 45-50, about 40-45, about 35-40, about 30-35, about 25-30, about 20-25, about 15-20, about 10-15, about 6-10 nucleotides, about 1-5 nucleotides, or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) downstream of the 5′ end or 5′ cap of the mRNA and the GC-rich RNA element is located upstream and adjacent to or within about 1-6 or about 1-10 nucleotides of a Kozak-like sequence or Kozak consensus sequence in the 5′ UTR.

In some embodiments, the structural RNA element comprising a stem loop is located 41 nucleotides downstream of the 5′ end or 5′ cap of the mRNA and the GC-rich element is located upstream and adjacent to or within about 1-6 or about 1-10 nucleotides of a Kozak-like sequence or Kozak consensus sequence in the 5′ UTR. In some embodiments, the structural RNA element comprising a stem loop is located 41 nucleotides downstream of the 5′ end or 5′ cap of the mRNA and the GC-rich element is located upstream and adjacent to a Kozak-like sequence or Kozak consensus sequence in the 5′ UTR. In some embodiments, the structural RNA element comprising a stem loop is located 6 nucleotides downstream of the 5′ end or 5′ cap of the mRNA and the GC-rich element is located upstream and adjacent to a Kozak-like sequence or Kozak consensus sequence in the 5′ UTR. In some embodiments, the structural RNA element comprising a stem loop is located 23 nucleotides downstream of the 5′ end or 5′ cap of the mRNA and the GC-rich RNA element is located upstream and adjacent to a Kozak-like sequence or Kozak consensus sequence in the 5′ UTR.

In some aspects, the disclosure provides an mRNA comprising: a 5′ cap, a 5′ untranslated region (UTR), a Kozak-like sequence, an initiation codon, a full open reading frame encoding a polypeptide, and a 3′ UTR, wherein the 5′ UTR comprises a GC-rich RNA element comprising a nucleotide sequence selected from the group consisting of: SEQ ID NO: 1, SEQ ID NO: 2, SEQ ID NO: 3, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27 and SEQ ID NO: 28, and wherein the 5′ UTR comprises a structural RNA element comprising a stem loop comprises a nucleotide sequence selected from the group consisting of: SEQ ID NO: 6 and SEQ ID NO: 47.

In some aspects, the disclosure provides an mRNA comprising: a 5′ cap, a 5′ untranslated region (UTR), a Kozak-like sequence, an initiation codon, a full open reading frame encoding a polypeptide, and a 3′ UTR, wherein the 5′ UTR comprises a GC-rich RNA element comprising a nucleotide sequence selected from the group consisting of: SEQ ID NO: 1, SEQ ID NO: 2, SEQ ID NO: 3, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27 and SEQ ID NO: 28, and wherein the 5′ UTR comprises a structural RNA element comprising a stem loop comprising the nucleotide sequence of SEQ ID NO: 6.

In some aspects, the disclosure provides an mRNA comprising: a 5′ cap, a 5′ untranslated region (UTR), a Kozak-like sequence, an initiation codon, a full open reading frame encoding a polypeptide, and a 3′ UTR, wherein the 5′ UTR comprises a GC-rich RNA element comprising a nucleotide sequence selected from the group consisting of: SEQ ID NO: 1, SEQ ID NO: 2, SEQ ID NO: 3, SEQ ID NO: 18, SEQ ID NO: 19, SEQ ID NO: 20, SEQ ID NO: 21, SEQ ID NO: 22, and SEQ ID NO: 23, and wherein the 5′ UTR comprises a structural RNA element comprising a stem loop comprising the nucleotide sequence of SEQ ID NO: 6.

In some aspects, the disclosure provides an mRNA comprising: a 5′ cap, a 5′ untranslated region (UTR), a Kozak-like sequence, an initiation codon, a full open reading frame encoding a polypeptide, and a 3′ UTR, wherein the 5′ UTR comprises a GC-rich RNA element comprising a nucleotide sequence of SEQ ID NO: 1, and wherein the 5′ UTR comprises a structural RNA element comprising a stem loop comprising the nucleotide sequence of SEQ ID NO: 6.

In some aspects, the disclosure provides an mRNA comprising: a 5′ cap, a 5′ untranslated region (UTR), a Kozak-like sequence, an initiation codon, a full open reading frame encoding a polypeptide, and a 3′ UTR, wherein the 5′ UTR comprises a GC-rich RNA element comprising a nucleotide sequence of SEQ ID NO: 2, and wherein the 5′ UTR comprises a structural RNA element comprising a stem loop comprising the nucleotide sequence of SEQ ID NO: 6.

In some aspects, the disclosure provides an mRNA comprising: a 5′ cap, a 5′ untranslated region (UTR), a Kozak-like sequence, an initiation codon, a full open reading frame encoding a polypeptide, and a 3′ UTR, wherein the 5′ UTR comprises a GC-rich RNA element comprising a nucleotide sequence of SEQ ID NO: 3, and wherein the 5′ UTR comprises a structural RNA element comprising comprising a stem loop the nucleotide sequence of SEQ ID NO: 6.

In some aspects, the disclosure provides an mRNA comprising: a 5′ cap, a 5′ untranslated region (UTR), a Kozak-like sequence, an initiation codon, a full open reading frame encoding a polypeptide, and a 3′ UTR, wherein the 5′ UTR comprises a GC-rich RNA element comprising a nucleotide sequence of SEQ ID NO: 18, and wherein the 5′ UTR comprises a structural RNA element comprising a stem loop comprising the nucleotide sequence of SEQ ID NO: 6.

In some aspects, the disclosure provides an mRNA comprising: a 5′ cap, a 5′ untranslated region (UTR), a Kozak-like sequence, an initiation codon, a full open reading frame encoding a polypeptide, and a 3′ UTR, wherein the 5′ UTR comprises a GC-rich RNA element comprising a nucleotide sequence of SEQ ID NO: 19, and wherein the 5′ UTR comprises a structural RNA element comprising a stem loop comprising the nucleotide sequence of SEQ ID NO: 6.

In some aspects, the disclosure provides an mRNA comprising: a 5′ cap, a 5′ untranslated region (UTR), a Kozak-like sequence, an initiation codon, a full open reading frame encoding a polypeptide, and a 3′ UTR, wherein the 5′ UTR comprises a GC-rich RNA element comprising a nucleotide sequence of SEQ ID NO: 20, and wherein the 5′ UTR comprises a structural RNA element comprising a stem loop comprising the nucleotide sequence of SEQ ID NO: 6.

In some aspects, the disclosure provides an mRNA comprising: a 5′ cap, a 5′ untranslated region (UTR), a Kozak-like sequence, an initiation codon, a full open reading frame encoding a polypeptide, and a 3′ UTR, wherein the 5′ UTR comprises a GC-rich RNA element comprising a nucleotide sequence of SEQ ID NO: 21, and wherein the 5′ UTR comprises a structural RNA element comprising a stem loop comprising the nucleotide sequence of SEQ ID NO: 6.

In some aspects, the disclosure provides an mRNA comprising: a 5′ cap, a 5′ untranslated region (UTR), a Kozak-like sequence, an initiation codon, a full open reading frame encoding a polypeptide, and a 3′ UTR, wherein the 5′ UTR comprises a GC-rich RNA element comprising a nucleotide sequence of SEQ ID NO: 22, wherein n=2 or more, and wherein the 5′ UTR comprises a structural RNA element comprising a stem loop comprising the nucleotide sequence of SEQ ID NO: 6.

In some aspects, the disclosure provides an mRNA comprising: a 5′ cap, a 5′ untranslated region (UTR), a Kozak-like sequence, an initiation codon, a full open reading frame encoding a polypeptide, and a 3′ UTR, wherein the 5′ UTR comprises a GC-rich RNA element comprising a nucleotide sequence of SEQ ID NO: 23, wherein n=2 or more, and wherein the 5′ UTR comprises a structural RNA element comprising a stem loop comprising the nucleotide sequence of SEQ ID NO: 6.

In some aspects, the disclosure provides an mRNA comprising: a 5′ cap, a 5′ untranslated region (UTR), a Kozak-like sequence, an initiation codon, a full open reading frame encoding a polypeptide, and a 3′ UTR, wherein the 5′ UTR comprises a GC-rich RNA element comprising a nucleotide sequence of SEQ ID NO: 1, wherein the GC-rich RNA element is located upstream and adjacent to or within about 1-6 or about 1-10 nucleotides of a Kozak-like sequence or Kozak consensus sequence in the 5′ UTR, and wherein the 5′ UTR comprises a structural RNA element comprising a stem loop comprising the nucleotide sequence of SEQ ID NO: 6.

In some embodiments, the structural RNA element comprising a stem loop comprises the nucleotide sequence of SEQ ID NO: 6, where the structure RNA element is located about 45-50, about 40-45, about 35-40, about 30-35, about 25-30, about 20-25, about 15-20, about 10-15, about 6-10 nucleotides, about 1-5 nucleotides, or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) upstream of, or is upstream and adjacent to the GC-rich RNA element comprising the nucleotide sequence of SEQ ID NO: 1 in the 5′ UTR.

In some aspects, the disclosure provides an mRNA comprising: a 5′ cap, a 5′ untranslated region (UTR), a Kozak-like sequence, an initiation codon, a full open reading frame encoding a polypeptide, and a 3′ UTR, wherein the 5′ UTR comprises a GC-rich RNA element comprising a nucleotide sequence of SEQ ID NO: 1, wherein the GC-rich RNA element is located upstream and adjacent to a Kozak-like sequence or Kozak consensus sequence in the 5′ UTR, wherein the 5′ UTR comprises a structural RNA element comprising a stem loop comprising the nucleotide sequence of SEQ ID NO: 6, and wherein the structural RNA element is located upstream and adjacent to the GC-rich RNA element.

In some aspects, the disclosure provides an mRNA comprising: a 5′ cap, a 5′ untranslated region (UTR), a Kozak-like sequence, an initiation codon, a full open reading frame encoding a polypeptide, and a 3′ UTR, wherein the 5′ UTR comprises a GC-rich RNA element comprising a nucleotide sequence of SEQ ID NO: 1, wherein the GC-rich RNA element is located upstream and adjacent to a Kozak-like sequence or Kozak consensus sequence in the 5′ UTR, wherein the 5′ UTR comprises a structural RNA element comprising a stem loop comprising the nucleotide sequence of SEQ ID NO: 6, and wherein the structural RNA element is located 35 nucleotides upstream of the GC-rich RNA element.

In some aspects, the disclosure provides an mRNA comprising: a 5′ cap, a 5′ untranslated region (UTR), a Kozak-like sequence, an initiation codon, a full open reading frame encoding a polypeptide, and a 3′ UTR, wherein the 5′ UTR comprises a GC-rich RNA element comprising a nucleotide sequence of SEQ ID NO: 1, wherein the GC-rich RNA element is located upstream and adjacent to a Kozak-like sequence or Kozak consensus sequence in the 5′ UTR, wherein the 5′ UTR comprises a structural RNA element comprising a stem loop comprising the nucleotide sequence of SEQ ID NO: 6, and wherein the structural RNA element is located 18 nucleotides upstream of the GC-rich RNA element.

In some aspects, the disclosure provides an mRNA comprising: a 5′ cap, a 5′ untranslated region (UTR), a Kozak-like sequence, an initiation codon, a full open reading frame encoding a polypeptide, and a 3′ UTR, wherein the 5′ UTR comprises a GC-rich RNA element and a structural RNA element comprising a stem loop, and wherein the 5′ UTR comprises a nucleotide sequence selected from the group consisting of: SEQ ID NO: 116; SEQ ID NO: 120; and SEQ ID NO: 124.

In some aspects, the disclosure provides an mRNA comprising: a 5′ cap, a 5′ untranslated region (UTR), a Kozak-like sequence, an initiation codon, a full open reading frame encoding a polypeptide, and a 3′ UTR, wherein the 5′ UTR comprises a GC-rich RNA element and a structural RNA element comprising a stem loop, and wherein the 5′ UTR comprises the nucleotide sequence of SEQ ID NO: 116.

In some aspects, the disclosure provides an mRNA comprising: a 5′ cap, a 5′ untranslated region (UTR), a Kozak-like sequence, an initiation codon, a full open reading frame encoding a polypeptide, and a 3′ UTR, wherein the 5′ UTR comprises a GC-rich RNA element and a structural RNA element comprising a stem loop, and wherein the 5′ UTR comprises the nucleotide sequence of SEQ ID NO: 120.

In some aspects, the disclosure provides an mRNA comprising: a 5′ cap, a 5′ untranslated region (UTR), a Kozak-like sequence, an initiation codon, a full open reading frame encoding a polypeptide, and a 3′ UTR, wherein the 5′ UTR comprises a GC-rich RNA element and a structural RNA element comprising a stem loop, and wherein the 5′ UTR comprises the nucleotide sequence of SEQ ID NO: 124.

### C-Rich RNA Element+Structural RNA Element

In some aspects, the disclosure provides an mRNA comprising a 5′ UTR comprising both a C-rich RNA element and a structural RNA element comprising a stem loop, such as those described herein. In some embodiments, a 5′ UTR comprising both a C-rich RNA element and a structural RNA element comprising a stem loop provides an enhanced translational regulatory activity compared to a 5′ UTR comprising a C-rich RNA element or a structural RNA element alone. In some embodiments, the amount or extent of leaky scanning of an mRNA comprising a 5′ UTR comprising both a C-rich RNA element and a structural RNA element of the disclosure is additively or synergistically decreased.

In some embodiments, leaky scanning of an mRNA comprising a 5′UTR comprising a C-rich RNA element and a structural RNA element comprising a stem loop of the disclosure is reduced by about 1-fold, about 2-fold, about 3-fold, about 4-fold, about 5-fold, about 6-fold, about 7-fold, about 8-fold, about 9-fold, about 10-fold relative to the leaky scanning of an mRNA comprising a 5′UTR comprising a C-rich RNA element alone or an mRNA comprising a 5′UTR comprising a structural RNA element alone. In some embodiments, leaky scanning of an mRNA comprising a 5′UTR comprising a C-rich RNA element and a structural RNA element of the disclosure is reduced by about 1-fold, about 2-fold, about 3-fold, about 4-fold, about 5-fold, about 6-fold, about 7-fold, about 8-fold, about 9-fold, about 10-fold relative to the leaky scanning of an mRNA comprising a 5′UTR without a C-rich RNA element or a structural RNA element. In some embodiments, the leaky scanning of an mRNA comprising a 5′UTR comprising a C-rich RNA element and a structural RNA element is reduced by about 5%, about 10%, about 15%, about 20%, about 30%, about 40%, about 50%, about 60%, about 70%, about 80%, about 90% or about 100% relative to the leaky scanning of an mRNA comprising a 5′UTR comprising a C-rich RNA element alone or an mRNA comprising a 5′UTR comprising a structural RNA element alone. In some embodiments, the leaky scanning of an mRNA comprising a 5′UTR comprising a C-rich RNA element and a structural RNA element is reduced by about 5%, about 10%, about 15%, about 20%, about 30%, about 40%, about 50%, about 60%, about 70%, about 80%, about 90% or about 100% relative to the leaky scanning of an mRNA comprising a 5′UTR without a C-rich RNA element or a structural RNA element. In some embodiments, the leaky scanning of an mRNA comprising a C-rich RNA element and a structural RNA element is abolished or undetectable.

In some aspects, the disclosure provides an mRNA comprising one or more C-rich RNA elements (e.g., 2, 3, 4) and one or more structural RNA elements comprising a stem loop (e.g., 2, 3, 4).

In some embodiments, the disclosure provides an mRNA having a C-rich RNA element and a structural RNA element comprising a stem loop as described herein, wherein the C-rich RNA element and the structural RNA element precede a Kozak-like sequence or Kozak consensus sequence in the 5′ UTR. In some embodiments, the disclosure provides an mRNA having a C-rich RNA element and a structural RNA element comprising a stem loop as described herein, wherein the C-rich RNA element and the structural RNA element are located upstream of a Kozak-like sequence or Kozak consensus sequence in the 5′ UTR.

In some embodiments, the C-rich RNA element is upstream the structural RNA element in the 5′UTR. In some embodiments, the C-rich RNA element is downstream of the structural RNA element in the 5′UTR. In some embodiments, the C-rich RNA element is proximal to the 5′ end or 5′ cap of the mRNA relative to the location of the structural RNA element in the 5′ UTR. In some embodiments, the C-rich RNA element is proximal to a Kozak-like sequence or Kozak consensus sequence relative to the location of the structural RNA element in the 5′ UTR.

In some embodiments, the C-rich RNA element is located about 45-50, about 40-45, about 35-40, about 30-35, about 25-30, about 20-25, about 15-20, about 10-15, about 6-10 nucleotides, about 1-5 nucleotides, or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) downstream of the 5′ end or 5′ cap of the mRNA and the structural RNA element is located downstream of the C-rich RNA element in the 5′ UTR.

In some embodiments, the C-rich RNA element is located adjacent to the 5′ end or 5′ cap of the mRNA and the structural RNA element comprising a stem loop is located downstream of the C-rich RNA element in the 5′ UTR. In some embodiments, the C-rich RNA element is located 6 nucleotides downstream of the 5′ end or 5′ cap of the mRNA and the structural RNA element is located downstream of the C-rich RNA element.

In some aspects, the disclosure provides an mRNA comprising: a 5′ cap, a 5′ untranslated region (UTR), a Kozak-like sequence, an initiation codon, a full open reading frame encoding a polypeptide, and a 3′ UTR, wherein the 5′ UTR comprises a C-rich RNA element comprising a nucleotide sequence selected from the group consisting of: SEQ ID NO: 29, SEQ ID NO: 30, SEQ ID NO: 31, SEQ ID NO: 32, SEQ ID NO: 33 and SEQ ID NO: 34, and wherein the 5′ UTR comprises a structural RNA element comprising a stem loop comprising the nucleotide sequence of SEQ ID NO: 6.

In some embodiments, the C-rich RNA element comprising a nucleotide sequence selected from the group consisting of: SEQ ID NO: 29, SEQ ID NO: 30, SEQ ID NO: 31, SEQ ID NO: 32, SEQ ID NO: 33 and SEQ ID NO: 34 is located about 45-50, about 40-45, about 35-40, about 30-35, about 25-30, about 20-25, about 15-20, about 10-15, about 6-10 nucleotides, about 1-5 nucleotides, or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) downstream of, or is downstream and adjacent to the 5′ end or 5′ cap of the mRNA and the structural RNA element comprising a stem loop comprising the nucleotide sequence of SEQ ID NO: 6 is located downstream of the C-rich RNA element in the 5′ UTR.

In some aspects, the disclosure provides an mRNA comprising: a 5′ cap, a 5′ untranslated region (UTR), a Kozak-like sequence, an initiation codon, a full open reading frame encoding a polypeptide, and a 3′ UTR, wherein the 5′ UTR comprises a C-rich RNA element comprising the nucleotide sequence of SEQ ID NO: 29, and wherein the 5′ UTR comprises a structural RNA element comprising a stem loop comprising the nucleotide sequence of SEQ ID NO: 6.

In some aspects, the disclosure provides an mRNA comprising: a 5′ cap, a 5′ untranslated region (UTR), a Kozak-like sequence, an initiation codon, a full open reading frame encoding a polypeptide, and a 3′ UTR, wherein the 5′ UTR comprises a C-rich RNA element comprising the nucleotide sequence of SEQ ID NO: 30, and wherein the 5′ UTR comprises a structural RNA element comprising a stem loop comprising the nucleotide sequence of SEQ ID NO: 6.

In some aspects, the disclosure provides an mRNA comprising: a 5′ cap, a 5′ untranslated region (UTR), a Kozak-like sequence, an initiation codon, a full open reading frame encoding a polypeptide, and a 3′ UTR, wherein the 5′ UTR comprises a C-rich RNA element comprising the nucleotide sequence of SEQ ID NO: 31, and wherein the 5′ UTR comprises a structural RNA element comprising a stem loop comprising the nucleotide sequence of SEQ ID NO: 6.

In some aspects, the disclosure provides an mRNA comprising: a 5′ cap, a 5′ untranslated region (UTR), a Kozak-like sequence, an initiation codon, a full open reading frame encoding a polypeptide, and a 3′ UTR, wherein the 5′ UTR comprises a C-rich RNA element comprising the nucleotide sequence of SEQ ID NO: 32, and wherein the 5′ UTR comprises a structural RNA element comprising a stem loop comprising the nucleotide sequence of SEQ ID NO: 6.

In some aspects, the disclosure provides an mRNA comprising: a 5′ cap, a 5′ untranslated region (UTR), a Kozak-like sequence, an initiation codon, a full open reading frame encoding a polypeptide, and a 3′ UTR, wherein the 5′ UTR comprises a C-rich RNA element comprising the nucleotide sequence of SEQ ID NO: 33, and wherein the 5′ UTR comprises a structural RNA element comprising a stem loop comprising the nucleotide sequence of SEQ ID NO: 6.

In some aspects, the disclosure provides an mRNA comprising: a 5′ cap, a 5′ untranslated region (UTR), a Kozak-like sequence, an initiation codon, a full open reading frame encoding a polypeptide, and a 3′ UTR, wherein the 5′ UTR comprises a C-rich RNA element comprising the nucleotide sequence of SEQ ID NO: 34, and wherein the 5′ UTR comprises a structural RNA element comprising a stem loop comprising the nucleotide sequence of SEQ ID NO: 6.

### GC-Rich RNA Element+C-Rich RNA Element+Structural RNA Element

In some aspects, the disclosure provides an mRNA comprising a 5′ UTR comprising a combination of a GC-rich RNA element, a C-rich RNA element, and a structural RNA element comprising a stem loop, as described herein. In some embodiments, a 5′ UTR comprising a combination of a GC-rich RNA element, a C-rich RNA element, and a structural RNA element comprising a stem loop provides an enhanced translational regulatory activity compared to a 5′ UTR comprising a GC-rich RNA element, or a C-rich RNA element, or a structural RNA element, or compared to a 5′ UTR comprising a combination of a GC-rich RNA element and a C-rich RNA element, or a combination of a GC-rich RNA element and a structural RNA element, or a combination of a C-rich RNA element and a structural RNA element. In some embodiments, the amount or extent of leaky scanning of an mRNA comprising a 5′ UTR comprising a combination of a GC-rich RNA element, a C-rich RNA element, and a structural RNA element of the disclosure is additively or synergistically decreased.

In some embodiments, leaky scanning of an mRNA comprising a 5′UTR comprising a combination of a GC-rich RNA element, a C-rich RNA element, and a structural RNA element of the disclosure is reduced by about 1-fold, about 2-fold, about 3-fold, about 4-fold, about 5-fold, about 6-fold, about 7-fold, about 8-fold, about 9-fold, or about 10-fold relative to the leaky scanning of an mRNA comprising a 5′UTR comprising a GC-rich RNA element alone, or a C-rich RNA element alone, or a structural RNA element alone, or of an mRNA comprising a comprising a 5′ UTR comprising a combination of a combination of a GC-rich RNA element and a C-rich RNA element, or a combination of a GC-rich RNA element and a structural RNA element, or a combination of a C-rich RNA element and a structural RNA element.

In some embodiments, leaky scanning of an mRNA comprising a 5′UTR comprising a combination of a GC-rich RNA element, a C-rich RNA element, and a structural RNA element of the disclosure is reduced by about 1-fold, about 2-fold, about 3-fold, about 4-fold, about 5-fold, about 6-fold, about 7-fold, about 8-fold, about 9-fold, or about 10-fold relative to the leaky scanning of an mRNA comprising a 5′UTR without a GC-rich RNA element, a C-rich RNA element, or a structural RNA element.

In some embodiments, the leaky scanning of an mRNA comprising a 5′UTR comprising a combination of a GC-rich RNA element, a C-rich RNA element, and a structural RNA element is reduced by about 5%, about 10%, about 15%, about 20%, about 30%, about 40%, about 50%, about 60%, about 70%, about 80%, about 90% or about 100% relative to the leaky scanning of an mRNA comprising a 5′UTR comprising a GC-rich RNA element alone, or a C-rich RNA element alone, or a structural RNA element alone, or of an mRNA comprising a 5′ UTR comprising a combination of a GC-rich RNA element and a C-rich RNA element, or a combination of a GC-rich RNA element and a structural RNA element, or a combination of a C-rich RNA element and a structural RNA element.

In some embodiments, the leaky scanning of an mRNA comprising a 5′UTR comprising a combination of a GC-rich RNA element, a C-rich RNA element, and a structural RNA element is reduced by about 5%, about 10%, about 15%, about 20%, about 30%, about 40%, about 50%, about 60%, about 70%, about 80%, about 90% or about 100% relative to the leaky scanning of an mRNA comprising a 5′UTR comprising without a GC-rich RNA element, a C-rich RNA element, or a structural RNA element. In some embodiments, the leaky scanning of an mRNA comprising a combination of a GC-rich RNA element, a C-rich RNA element, and a structural RNA element is abolished or undetectable.

In some aspects, the disclosure provides an mRNA comprising one or more GC-rich RNA elements (e.g., 2, 3, 4), one or more C-rich RNA element (e.g., 2, 3, 4), and one or more structural RNA elements comprising a stem loop (e.g., 2, 3, 4).

In some aspects, the disclosure provides an mRNA, wherein the mRNA comprises: a 5′ cap, a 5′ untranslated region (UTR), a Kozak-like sequence, an initiation codon, a full open reading frame encoding a polypeptide, and a 3′ UTR, wherein the 5′ UTR comprises a C-rich RNA element comprising a nucleotide sequence selected from the group consisting of: SEQ ID NO: 29, SEQ ID NO: 30, SEQ ID NO: 31, SEQ ID NO: 32, SEQ ID NO: 33 and SEQ ID NO: 34, and comprises a GC-rich RNA element comprising a nucleotide sequence selected from the group consisting of: SEQ ID NO: 1, SEQ ID NO: 2, SEQ ID NO: 3, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27 and SEQ ID NO: 28, and comprises a structural RNA element comprising a stem loop comprising the nucleotide sequence of SEQ ID NO: 6.

In some embodiments, the C-rich RNA element comprises a nucleotide sequence selected from the group consisting of SEQ ID NO: 31, SEQ ID NO: 32 and SEQ ID NO: 33, and the GC-rich RNA element comprises a nucleotide sequence selected from the group consisting of: SEQ ID NO: 1, SEQ ID NO: 2 and SEQ ID NO: 23.

In some aspects, the disclosure provides an mRNA, wherein the mRNA comprises: a 5′ cap, a 5′ untranslated region (UTR), a Kozak-like sequence, an initiation codon, a full open reading frame encoding a polypeptide, and a 3′ UTR, wherein the 5′ UTR comprises a C-rich RNA element comprising the nucleotide sequence set forth in SEQ ID NO: 29, a GC-rich RNA element comprising the nucleotide sequence set forth in SEQ ID NO: 1, and a structural RNA element comprising a stem loop comprising the nucleotide sequence of SEQ ID NO: 6.

In some aspects, the disclosure provides an mRNA, wherein the mRNA comprises: a 5′ cap, a 5′ untranslated region (UTR), a Kozak-like sequence, an initiation codon, a full open reading frame encoding a polypeptide, and a 3′ UTR, wherein the 5′ UTR comprises a C-rich RNA element comprising the nucleotide sequence set forth in SEQ ID NO: 30, a GC-rich RNA element comprising the nucleotide sequence set forth in SEQ ID NO: 1, and a structural RNA element comprising a stem loop comprising the nucleotide sequence of SEQ ID NO: 6.

In some aspects, the disclosure provides an mRNA, wherein the mRNA comprises: a 5′ cap, a 5′ untranslated region (UTR), a Kozak-like sequence, an initiation codon, a full open reading frame encoding a polypeptide, and a 3′ UTR, wherein the 5′ UTR comprises a C-rich RNA element comprising the nucleotide sequence set forth in SEQ ID NO: 31, a GC-rich RNA element comprising the nucleotide sequence set forth in SEQ ID NO: 1, and a structural RNA element comprising a stem loop comprising the nucleotide sequence of SEQ ID NO: 6.

In some aspects, the disclosure provides an mRNA, wherein the mRNA comprises: a 5′ cap, a 5′ untranslated region (UTR), a Kozak-like sequence, an initiation codon, a full open reading frame encoding a polypeptide, and a 3′ UTR, wherein the 5′ UTR comprises a C-rich RNA element comprising the nucleotide sequence set forth in SEQ ID NO: 32, a GC-rich RNA element comprising the nucleotide sequence set forth in SEQ ID NO: 1, and a structural RNA element comprising a stem loop comprising the nucleotide sequence of SEQ ID NO: 6.

In some aspects, the disclosure provides an mRNA, wherein the mRNA comprises: a 5′ cap, a 5′ untranslated region (UTR), a Kozak-like sequence, an initiation codon, a full open reading frame encoding a polypeptide, and a 3′ UTR, wherein the 5′ UTR comprises a C-rich RNA element comprising the nucleotide sequence set forth in SEQ ID NO: 33, a GC-rich RNA element comprising the nucleotide sequence set forth in SEQ ID NO: 1, and a structural RNA element comprising a stem loop comprising the nucleotide sequence of SEQ ID NO: 6.

In some aspects, the disclosure provides an mRNA, wherein the mRNA comprises: a 5′ cap, a 5′ untranslated region (UTR), a Kozak-like sequence, an initiation codon, a full open reading frame encoding a polypeptide, and a 3′ UTR, wherein the 5′ UTR comprises a C-rich RNA element comprising the nucleotide sequence set forth in SEQ ID NO: 34, a GC-rich RNA element comprising the nucleotide sequence set forth in SEQ ID NO: 1, and a structural RNA element comprising a stem loop comprising the nucleotide sequence of SEQ ID NO: 6.

In some aspects, the disclosure provides an mRNA, wherein the mRNA comprises a 5′ UTR comprising a combination of a GC-rich RNA element, a C-rich RNA element, and a structural RNA element comprising a stem loop, wherein the 5′UTR comprises the nucleotide sequence set forth in SEQ ID NO: 128.

In some embodiments, the disclosure provides an mRNA, wherein the mRNA comprises a mRNA comprises a 5′ UTR comprising a combination of a GC-rich RNA element, a C-rich RNA element, and a structural RNA element comprising a stem loop, wherein the 5′UTR comprises the nucleotide sequence set forth in SEQ ID NO: 132.

In some embodiments, the disclosure provides an mRNA, wherein the mRNA comprises a mRNA comprises a 5′ UTR comprising a combination of a GC-rich RNA element, a C-rich RNA element, and a structural RNA element comprising a stem loop, wherein the 5′UTR comprises the nucleotide sequence set forth in SEQ ID NO: 136.

In some embodiments, the disclosure provides an mRNA, wherein the mRNA comprises a mRNA comprises a 5′ UTR comprising a combination of a GC-rich RNA element, a C-rich RNA element, and a structural RNA element comprising a stem loop, wherein the 5′UTR comprises the nucleotide sequence set forth in SEQ ID NO: 140.

In some embodiments, the disclosure provides an mRNA, wherein the mRNA comprises a mRNA comprises a 5′ UTR comprising a combination of a GC-rich RNA element, a C-rich RNA element, and a structural RNA element comprising a stem loop, wherein the 5′UTR comprises the nucleotide sequence set forth in SEQ ID NO: 144.

TABLE 4
Exemplary 5′ UTRs with GC-Rich RNA Elements, C-Rich RNA
Elements, and Structural RNA Elements

| | | SEQ ID |
|---|---|---|
| 5′ UTRs | Sequence | NO |
| F593 | GGGAAACCCCCCACCCCCGUAAGAGAGAAAAGAAGAGUA | 128 |
| | AGAAGAAAUAUAAGAUCUCCCUGAGCUUCAGGGAGCCC | |
| | CGGCGCC[GCCACC] | |
| combo1_P2_p2 | GGGAAACCCCCCACCCCCGUCUCCCUGAGCUUCAGGGAG | 132 |
| | UAAGAGAGAAAAGAAGAGUAAGAAGAAAUAUAAGACCCC | |
| | GGCGCC[GCCACC] | |
| combo1_P2_p3 | GGGAAACCCCCCACCCCCGUAAGAGAGAAAAGAAGAUCU | 136 |
| | CCCUGAGCUUCAGGGAGGUAAGAAGAAAUAUAAGACCC | |
| | CGGCGCC[GCCACC] | |
| combo2_P2_p2 | GGGAAAUCCCCACAACCGUCUCCCUGAGCUUCAGGGAG | 140 |
| | UAAGAGAGAAAAGAAGAGUAAGAAGAAAUAUAAGACCCC | |
| | GGCGCC[GCCACC] | |
| combo2_P2_p3 | GGGAAAUCCCCACAACCGUAAGAGAGAAAAGAAGAUCUC | 144 |
| | CCUGAGCUUCAGGGAGGUAAGAAGAAAUAUAAGACCCC | |
| | GGCGCC[GCCACC] | |

(C-rich RNA Element underlined; Structural RNA Element bold; GC-rich RNA Element italicized, Kozak sequence [bracket])

5′ UTRs Comprising C-Rich and/or GC-Rich RNA Elements

In some aspects, the disclosure provides mRNAs having RNA elements (e.g., C-rich, GC-rich RNA, structural RNA elements and combinations thereof) which provide a desired translational regulatory activity to the mRNA. In one aspect, the mRNAs of the disclosure comprise a 5′ UTR comprising a C-rich RNA element, a GC-rich RNA element, or a combination thereof, as described herein, wherein the addition of the C-rich RNA element, the GC-rich RNA element, or the combination thereof, provides one or more translational regulatory activities described herein (e.g. inhibition of leaky scanning). In some embodiments, an mRNA provided by the disclosure comprises a 5′ UTR comprising a C-rich RNA element described herein, wherein the C-rich RNA element provides one or more translational regulatory activities described herein (e.g., inhibition of leaky scanning). In some embodiments, an mRNA provided by the disclosure comprises a 5′ UTR comprising a C-rich RNA element and a GC-rich RNA element of the disclosure, wherein the C-rich RNA element and GC-rich RNA element provide one or more translational regulatory activities described herein (e.g., inhibition of leaky scanning). Translational regulatory activities provided by the C-rich RNA element, GC-rich RNA element, or combination thereof, includes promoting translation of only one open reading frame encoding a desired polypeptide or translation product, or reducing, inhibiting or eliminating the failure to initiate translation of the therapeutic protein or peptide at a desired initiator codon, as a consequence of leaky scanning or other mechanisms.

In some embodiments, the mRNAs of the disclosure comprise a 5′ UTR to which a C-rich RNA element, a GC-rich RNA element, or a combination thereof, described herein, is added or inserted, thereby reducing leaky scanning of the 5′ UTR by the cellular translation machinery. In some embodiments, the mRNAs provided by the disclosure comprise a core 5′ UTR nucleotide sequence to which a C-rich RNA element, a GC-rich RNA element, or a combination thereof, described herein is added, thereby reducing leaky scanning of the 5′ UTR by the cellular translation machinery. In some embodiments, the core 5′ UTR comprises the nucleotide sequence set forth in SEQ ID NO: 45. In some embodiments, the core 5′ UTR comprises the nucleotide sequence set forth in SEQ ID NO: 46.

In one aspect, the mRNA of the disclosure comprises a 5′ UTRs comprising the nucleotide set forth in SEQ ID NO: 4 in which a C-rich RNA element and a GC-rich RNA element is inserted.

In one aspect, the mRNA of the disclosure comprises a 5′ UTRs comprising the nucleotide set forth in SEQ ID NO: 62 in which a C-rich RNA element and a GC-rich RNA element is inserted.

In one aspect, the mRNA of the disclosure comprises a 5′ UTRs comprising the nucleotide selected from SEQ ID NO: 65, SEQ ID NO: 68, SEQ ID NO: 13, SEQ ID NO: 14, SEQ ID NO: 15, or SEQ ID NO: 16 in which a C-rich RNA element and a GC-rich RNA element is inserted.

In one aspect, the mRNA of the disclosure comprises a 5′ UTRs comprising the nucleotide set forth in SEQ ID NO: 43 in which a C-rich RNA element and, optionally, a GC-rich RNA element is inserted.

In one aspect, the mRNA of the disclosure comprises a 5′ UTRs comprising the nucleotide set forth in SEQ ID NO: 45 in which a C-rich RNA element and, optionally, a GC-rich RNA element is inserted.

In one aspect, the mRNA of the disclosure comprises a 5′ UTRs comprising the nucleotide set forth in SEQ ID NO: 8 in which a C-rich RNA element and, optionally, a GC-rich RNA element is inserted.

In one aspect, the mRNA of the disclosure comprises a 5′ UTRs comprising the nucleotide set forth in SEQ ID NO: 46 in which a C-rich RNA element and, optionally, a GC-rich RNA element is inserted.

In one aspect, the mRNA of the disclosure comprises a 5′ UTRs comprising the nucleotide set forth in SEQ ID NO: 42 in which a C-rich RNA element and, optionally, a GC-rich RNA element is inserted.

In one aspect, the mRNA of the disclosure comprises a 5′ UTRs comprising the nucleotide set forth in SEQ ID NO: 39 in which a C-rich RNA element and, optionally, a GC-rich RNA element is inserted.

Exemplary 5′ UTRs comprising C-rich RNA elements, GC-rich elements, and combinations thereof provided by the disclosure are set forth in Table 5. These 5′ UTRs are useful in the mRNAs of the disclosure.

TABLE 5
Exemplary 5′UTRs and 5′UTRs with GC-Rich RNA Elements
(GC-Rich Elements italicized)

| | | SEQ ID |
|---|---|---|
| 5′ UTRs | Sequence | NO |
| 5′v1.0 (DNA) | GGGAAATAAGAGAGAAAAGAAGAGTAAGAAGAAATATAA | 58 |
| | GAGCCACC | |
| 5′v1.0 (RNA) | GGGAAAUAAGAGAGAAAAGAAGAGUAAGAAGAAAUAUA | 45 |
| | AGAGCCACC | |
| 5′v1.0 Core (DNA) | TAAGAGAGAAAAGAAGAGTAAGAAGAAATATAAGA | 55 |
| 5′v1.0 Core (RNA) | UAAGAGAGAAAAGAAGAGUAAGAAGAAAUAUAAGA | 8 |
| 5′v1.1 (DNA) | GGGAAATAAGAGAGAAAAGAAGAGTAAGAAGAAATATAA | 9 |
| | GACCCCGGCGCCGCCACC | |
| 5′v1.1 | GGGAAAUAAGAGAGAAAAGAAGAGUAAGAAGAAAUAUA | 4 |
| (RNA) | AGACCCCGGCGCCGCCACC | |
| V2-5′UTR (DNA) | GGGAAATAAGAGAGAAAAGAAGAGTAAGAAGAAATATAA | 10 |
| | GACCCCGGCGCCACC | |
| V2-5′UTR (RNA) | GGGAAAUAAGAGAGAAAAGAAGAGUAAGAAGAAAUAUA | 62 |
| | AGACCCCGGCGCCACC | |
| CG1-5′UTR | GGGAAATAAGAGAGAAAAGAAGAGTAAGAAGAAATATAA | 11 |
| (DNA) | GAGCGCCCCGCGGCGCCCCGCGGCCACC | |
| CG1-5′UTR | GGGAAAUAAGAGAGAAAAGAAGAGUAAGAAGAAAUAUA | 65 |
| (RNA) | AGAGCGCCCCGCGGCGCCCCGCGGCCACC | |
| CG2-5′UTR | GGGAAATAAGAGAGAAAAGAAGAGTAAGAAGAAATATAA | 12 |
| (DNA) | GACCCGCCCGCCCCGCCCCGCCGCCACC | |
| CG2-5′UTR | GGGAAAUAAGAGAGAAAAGAAGAGUAAGAAGAAAUAUA | 68 |
| (RNA) | AGACCCGCCCGCCCCGCCCCGCCGCCACC | |
| KT1-UTR | GGGCCCGCCGCCAAC | 13 |
| KT2-UTR | GGGCCCGCCGCCACC | 14 |
| KT3-UTR | GGGCCCGCCGCCGAC | 15 |
| KT4-UTR | GGGCCCGCCGCCGCC | 16 |
| GCC3-ExtKozak | GGGAAAGCCGCCGCCGCCACC | 43 |
| (Ref) | | |
| S065 core (DNA) | CCTCATATCCAGGCTCAAGAATAGAGCTCAGTGTTTTGTTG | 87 |
| | TTTAATCATTCCGACGTGTTTTGCGATATTCGCGCAAAGCA | |
| | GCCAGTCGCGCGCTTGCTTTTAAGTAGAGTTGTTTTTCCAC | |
| | CCGTTTGCCAGGCATCTTTAATTTAACATATTTTTATTTTTC | |
| | AGGCTAACCTA | |
| S065 core (RNA) | CCUCAUAUCCAGGCUCAAGAAUAGAGCUCAGUGUUUUGU | 46 |
| | UGUUUAAUCAUUCCGACGUGUUUUGCGAUAUUCGCGCAA | |
| | AGCAGCCAGUCGCGCGCUUGCUUUUAAGUAGAGUUGUUU | |
| | UUCCACCCGUUUGCCAGGCAUCUUUAAUUUAACAUAUUU | |
| | UUAUUUUUCAGGCUAACCUA | |
| S065 (DNA) | GGGAGACCTCATATCCAGGCTCAAGAATAGAGCTCAGTGT | 88 |
| | TTTGTTGTTTAATCATTCCGACGTGTTTTGCGATATTCGCG | |
| | CAAAGCAGCCAGTCGCGCGCTTGCTTTTAAGTAGAGTTGT | |
| | TTTTCCACCCGTTTGCCAGGCATCTTTAATTTAACATATTTT | |
| | TATTTTTCAGGCTAACCTAAAGCAGAGAA | |
| S065 (RNA) | GGGAGACCUCAUAUCCAGGCUCAAGAAUAGAGCUCAGUG | 42 |
| | UUUUGUUGUUUAAUCAUUCCGACGUGUUUUGCGAUAUU | |
| | CGCGCAAAGCAGCCAGUCGCGCGCUUGCUUUUAAGUAGA | |
| | GUUGUUUUUCCACCCGUUUGCCAGGCAUCUUUAAUUUAA | |
| | CAUAUUUUUAUUUUUCAGGCUAACCUAAAGCAGAGAA | |
| combo3_S065 | GGGAGACCTCATATCCAGGCTCAAGAATAGAGCTCAGTGT | 91 |
| (S065 core | TTTGTTGTTTAATCATTCCGACGTGTTTTGCGATATTCGCG | |
| extended Kozak) | CAAAGCAGCCAGTCGCGCGCTTGCTTTTAAGTAGAGTTGT | |
| (DNA) | TTTTCCACCCGTTTGCCAGGCATCTTTAATTTAACATATTTT | |
| | TATTTTTCAGGCTAACCTACGCCGCCACC | |
| combo3_S065 | GGGAGACCUCAUAUCCAGGCUCAAGAAUAGAGCUCAGUG | 39 |
| (S065 core | UUUUGUUGUUUAAUCAUUCCGACGUGUUUUGCGAUAUU | |
| extended Kozak) | CGCGCAAAGCAGCCAGUCGCGCGCUUGCUUUUAAGUAGA | |
| (RNA) | GUUGUUUUUCCACCCGUUUGCCAGGCAUCUUUAAUUUAA | |
| | CAUAUUUUUAUUUUUCAGGCUAACCUACGCCGCCACC | |

In other aspects, the mRNA of the disclosure comprises a 5′ UTRs comprising the nucleotide set forth in SEQ ID NO: 37 in which a C-rich RNA element and, optionally, a GC-rich RNA element is inserted.

In one aspect, the mRNA of the disclosure comprises a 5′ UTRs comprising the nucleotide set forth in SEQ ID NO: 38 in which a C-rich RNA element and, optionally, a GC-rich RNA element is inserted.

In one aspect, the mRNA of the disclosure comprises a 5′ UTRs comprising the nucleotide set forth in SEQ ID NO: 40 in which a C-rich RNA element and, optionally, a GC-rich RNA element is inserted.

In one aspect, the mRNA of the disclosure comprises a 5′ UTRs comprising the nucleotide set forth in SEQ ID NO: 41 in which a C-rich RNA element and, optionally, a GC-rich RNA element is inserted.

Exemplary 5′ UTRs comprising C-rich RNA elements, and combinations with GC-rich elements, provided by the disclosure are set forth in Table 6. These 5′ UTRs are useful in the mRNAs of the disclosure.

TABLE 6
Exemplary 5′ UTRs with C-Rich RNA Elements
(C-rich RNA element in underlined; Kozak
bracketed)

| | | SEQ |
|---|---|---|
| | | ID |
| 5′UTR | Sequence | NO |
| combo1_S065 | GGGAAACCCCCCACCCCCGCCUCAUAUCCAGGCUC | 37 |
| | AAGAAUAGAGCUCAGUGUUUUGUUGUUUAAUCA | |
| | UUCCGACGUGUUUUGCGAUAUUCGCGCAAAGCAG | |
| | CCAGUCGCGCGCUUGCUUUUAAGUAGAGUUGUUU | |
| | UUCCACCCGUUUGCCAGGCAUCUUUAAUUUAACA | |
| | UAUUUUUAUUUUUCAGGCUAACCUAAAGCAGAGA | |
| | A | |
| combo2_S065 | GGGAAAUCCCCACAACCGCCUCAUAUCCAGGCUC | 38 |
| | AAGAAUAGAGCUCAGUGUUUUGUUGUUUAAUCA | |
| | UUCCGACGUGUUUUGCGAUAUUCGCGCAAAGCAG | |
| | CCAGUCGCGCGCUUGCUUUUAAGUAGAGUUGUUU | |
| | UUCCACCCGUUUGCCAGGCAUCUUUAAUUUAACA | |
| | UAUUUUUAUUUUUCAGGCUAACCUAAAGCAGAGA | |
| | A | |
| combo4_S065 | GGGAAACCCCCCACCCCCGCCUCAUAUCCAGGCUC | 40 |
| | AAGAAUAGAGCUCAGUGUUUUGUUGUUUAAUCA | |
| | UUCCGACGUGUUUUGCGAUAUUCGCGCAAAGCAG | |
| | CCAGUCGCGCGCUUGCUUUUAAGUAGAGUUGUUU | |
| | UUCCACCCGUUUGCCAGGCAUCUUUAAUUUAACA | |
| | UAUUUUUAUUUUUCAGGCUAACCUACGCC[GCCAC | |
| | C] | |
| combo5_S065 | GGGAAAUCCCCACAACCGCCUCAUAUCCAGGCUC | 41 |
| | AAGAAUAGAGCUCAGUGUUUUGUUGUUUAAUCA | |
| | UUCCGACGUGUUUUGCGAUAUUCGCGCAAAGCAG | |
| | CCAGUCGCGCGCUUGCUUUUAAGUAGAGUUGUUU | |
| | UUCCACCCGUUUGCCAGGCAUCUUUAAUUUAACA | |
| | UAUUUUUAUUUUUCAGGCUAACCUACGCC[GCCAC | |
| | C] | |

In one aspect, the mRNA of the disclosure comprises a 5′ UTRs comprising the nucleotide set forth in SEQ ID NO: 35 in which a C-rich RNA element and a GC-rich RNA element is inserted.

In one aspect, the mRNA of the disclosure comprises a 5′ UTRs comprising the nucleotide set forth in SEQ ID NO: 36 in which a C-rich RNA element and, optionally, a GC-rich RNA element is inserted.

In one aspect, the mRNA of the disclosure comprises a 5′ UTRs comprising the nucleotide set forth in SEQ ID NO: 44 in which a C-rich RNA element and, optionally, a GC-rich RNA element is inserted.

Exemplary 5′ UTRs comprising C-rich RNA elements, and combinations with GC-rich elements, provided by the disclosure are set forth in Table 7. These 5′ UTRs are useful in the mRNAs of the disclosure.

TABLE 7
Exemplary 5′ UTRs with C-Rich RNA Elements and GC-Rich RNA Elements
(GC-Rich Elements italicized; C-rich RNA element in underlined;
Kozak bracketed)

| | | SEQ ID |
|---|---|---|
| 5′UTR | Sequence | NO |
| combo1_V1.1 | GGGAAACCCCCCACCCCCGGGGAAAUAAGAGAGAA | 35 |
| | AAGAAGAGUAAGAAGAAAUAUAAGACCCCGGCGCC | |
| | [GCCACC] | |
| combo2_V1.1 | GGGAAAUCCCCACAACCGGGGAAAUAAGAGAGAAA | 36 |
| | AGAAGAGUAAGAAGAAAUAUAAGACCCCGGCGCC | |
| | [GCCACC] | |
| CrichCR4 + GCC3- | GGGAAACCCCCCUAAGCCGCCGCCGCC[GCCACC] | 44 |
| ExtKozak | | |

### 5′ UTRs Comprising Combinations of RNA Elements

In some aspects, the disclosure provides mRNAs having RNA elements (e.g., C-rich RNA elements, GC-rich RNA elements, and/or structural RNA elements) which provide a desired translational regulatory activity to the mRNA. In one aspect, the mRNAs of the disclosure comprise a 5′ UTR described herein to which a C-rich RNA element, a GC-rich RNA element, a structural RNA element, or a combination thereof, described herein is added or inserted, wherein the addition of the C-rich RNA element, the GC-rich RNA element, the structural RNA element, or the combination thereof, provides one or more translational regulatory activities described herein (e.g., inhibition of leaky scanning).

In some embodiments, an mRNA provided by the disclosure comprises a 5′ UTR comprising a C-rich RNA element described herein, wherein the C-rich RNA element provides one or more translational regulatory activities described herein (e.g., inhibition of leaky scanning). In some embodiments, an mRNA provided by the disclosure comprises a 5′ UTR comprising a C-rich RNA element and a GC-rich RNA element of the disclosure, wherein the C-rich RNA element and GC-rich RNA element provide one or more translational regulatory activities described herein (e.g., inhibition of leaky scanning). In some embodiments, an mRNA provided by the disclosure comprises a 5′ UTR comprising a combination of a C-rich RNA element, a GC-rich RNA element, and a structural RNA element comprising a stem loop of the disclosure, wherein the combination provides one or more translational regulatory activities described herein (e.g., inhibition of leaky scanning).

Translational regulatory activities provided by the C-rich RNA element, the GC-rich RNA element, the structural RNA element, or combination thereof, includes promoting translation of only one open reading frame encoding a desired polypeptide or translation product, or reducing, inhibiting or eliminating the failure to initiate translation of the therapeutic protein or peptide at a desired initiator codon, as a consequence of leaky scanning or other mechanisms.

In some embodiments, the mRNAs of the disclosure comprise a 5′ UTR to which a C-rich RNA element, a GC-rich RNA element, a structural RNA element, or a combination thereof, described herein, is added or inserted, thereby reducing leaky scanning of the 5′ UTR by the cellular translation machinery. In some embodiments, the mRNAs provided by the disclosure comprise a core 5′ UTR nucleotide sequence to which a C-rich RNA element, a GC-rich RNA element, a structural RNA element, or a combination thereof, described herein is added, thereby reducing leaky scanning of the 5′ UTR by the cellular translation machinery. In some embodiments, the core 5′ UTR comprises the nucleotide sequence set forth in SEQ ID NO: 45. In some embodiments, the core 5′ UTR comprises the nucleotide sequence set forth in SEQ ID NO: 8. In some embodiments, the core 5′ UTR comprises the nucleotide sequence set forth in SEQ ID NO: 46.

In one aspect, an mRNA of the disclosure comprises a 5′ UTR comprising the nucleotide set forth in SEQ ID NO: 8 in which a GC-rich RNA element and a structural RNA element described herein are inserted. In one aspect, an mRNA of the disclosure comprises a 5′ UTR comprising the nucleotide set forth in SEQ ID NO: 45 in which a GC-rich RNA element and a structural RNA element described herein are inserted.

In one aspect, an mRNA of the disclosure comprises a 5′ UTR comprising the nucleotide sequence set forth in SEQ ID NO: 46 in which a GC-rich RNA element and a structural RNA element described herein are inserted. In another aspect, an mRNA of the disclosure comprises a 5′ UTR comprising the nucleotide sequence set forth in SEQ ID NO: 42 in which a GC-rich RNA element and a structural RNA element described herein are inserted. In another aspect, an mRNA of the disclosure comprises a 5′ UTR comprising the nucleotide sequence set forth in SEQ ID NO: 39 in which a GC-rich RNA element and a structural RNA element described herein are inserted.

In one aspect, an mRNA of the disclosure comprises: a 5′ cap, a 5′ untranslated region (5′ UTR), a Kozak-like sequence, an initiation codon, a full open reading frame (ORF) encoding a polypeptide, and a 3′ UTR, wherein the 5′ UTR comprises a structural RNA element described herein and a GC-rich RNA element described herein inserted within the 5′ UTR comprising the nucleotide sequence of SEQ ID NO: 45.

In one aspect, an mRNA of the disclosure comprises: a 5′ cap, a 5′ untranslated region (5′ UTR), a Kozak-like sequence, an initiation codon, a full open reading frame (ORF) encoding a polypeptide, and a 3′ UTR, wherein the 5′ UTR comprises a structural RNA element comprising the nucleotide sequence of SEQ ID NO: 6 and a GC-rich RNA element comprising the nucleotide sequence of SEQ ID NO: 1 inserted within the 5′ UTR comprising the nucleotide sequence of SEQ ID NO: 45.

In one aspect, an mRNA of the disclosure comprises: a 5′ cap, a 5′ untranslated region (5′ UTR), a Kozak-like sequence, an initiation codon, a full open reading frame (ORF) encoding a polypeptide, and a 3′ UTR, wherein the 5′ UTR comprises a structural RNA element described herein, a GC-rich RNA element described herein, and a C-rich RNA element described herein inserted within the 5′ UTR comprising the nucleotide sequence of SEQ ID NO: 45.

In one aspect, the disclosure provides an mRNA comprising a 5′ cap, a 5′ untranslated region (5′ UTR), a Kozak-like sequence, an initiation codon, a full open reading frame (ORF) encoding a polypeptide, and a 3′ UTR, wherein the 5′ UTR comprises a structural RNA element comprising the nucleotide sequence of SEQ ID NO: 6, a GC-rich RNA element comprising the nucleotide sequence of SEQ ID NO: 1, and a C-rich RNA element comprising the nucleotide sequence selected from the group consisting of: SEQ ID NO: 31 and SEQ ID NO: 33 inserted within the 5′ UTR comprising the nucleotide sequence of SEQ ID NO: 45.

In one aspect, an mRNA of the disclosure comprises a 5′ UTR comprising the nucleotide sequence set forth in SEQ ID NO: 4 in which a structural RNA element described herein is inserted. In another aspect, an mRNA of the disclosure comprises a 5′ UTR comprising the nucleotide sequence set forth in SEQ ID NO: 62 in which a structural RNA element described herein is inserted.

In one aspect, an mRNA of the disclosure comprises a 5′ UTR comprising a nucleotide sequence selected from SEQ ID NO: 65, SEQ ID NO: 68, SEQ ID NO: 13, SEQ ID NO: 14, SEQ ID NO: 15, or SEQ ID NO: 16 in which a structural RNA element described herein is inserted. In another aspect, an mRNA of the disclosure comprises a 5′ UTR comprising the nucleotide sequence set forth in SEQ ID NO: 43 in which a structural RNA element described herein is inserted.

In another aspect, the disclosure provides an mRNA comprising a 5′ cap, a 5′ untranslated region (5′ UTR), a Kozak-like sequence, an initiation codon, a full open reading frame (ORF) encoding a polypeptide, and a 3′ UTR, wherein the 5′ UTR comprises a structural RNA element comprising the nucleotide sequence of SEQ ID NO: 6 inserted within the 5′ UTR comprising the nucleotide sequence of SEQ ID NO: 4

In one aspect, an mRNA of the disclosure comprises a 5′ UTR comprising the nucleotide set forth in SEQ ID NO: 8 in which a C-rich RNA element and a structural RNA element described herein are inserted. In one aspect, an mRNA of the disclosure comprises a 5′ UTR comprising the nucleotide set forth in SEQ ID NO: 45 in which a C-rich RNA element and a structural RNA element described herein are inserted.

In one aspect, an mRNA of the disclosure comprises a 5′ UTR comprising the nucleotide sequence set forth in SEQ ID NO: 46 in which a C-rich RNA element and a structural RNA element described herein are inserted. In another aspect, an mRNA of the disclosure comprises a 5′ UTR comprising the nucleotide sequence set forth in SEQ ID NO: 42 in which a C-rich RNA element and a structural RNA element described herein are inserted. In another aspect, an mRNA of the disclosure comprises a 5′ UTR comprising the nucleotide sequence set forth in SEQ ID NO: 39 in which a C-rich RNA element and a structural RNA element described herein are inserted.

In one aspect, an mRNA of the disclosure comprises a 5′ UTR comprising the nucleotide sequence set forth in SEQ ID NO: 37 in which a structural RNA element comprising a stem loop described herein is inserted.

In one aspect, an mRNA of the disclosure comprises a 5′ UTR comprising the nucleotide sequence set forth in SEQ ID NO: 38 in which a structural RNA element comprising a stem loop described herein is inserted.

In one aspect, an mRNA of the disclosure comprises a 5′ UTR comprising the nucleotide sequence set forth in SEQ ID NO: 40 in which a structural RNA element comprising a stem loop described herein is inserted.

In one aspect, an mRNA of the disclosure comprises a 5′ UTR comprising the nucleotide sequence set forth in SEQ ID NO: 41 in which a structural RNA element comprising a stem loop described herein is inserted.

In one aspect, an mRNA of the disclosure comprises a 5′ UTR comprising the nucleotide set forth in SEQ ID NO: 8 in which a GC-rich RNA element, a C-rich RNA element, and a structural RNA element described herein are inserted. In one aspect, an mRNA of the disclosure comprises a 5′ UTR comprising the nucleotide set forth in SEQ ID NO: 45 in which a GC-rich RNA element, a C-rich RNA element, and a structural RNA element described herein are inserted.

In one aspect, an mRNA of the disclosure comprises a 5′ UTR comprising the nucleotide sequence set forth in SEQ ID NO: 46 in which a GC-rich RNA element, a C-rich RNA element, and a structural RNA element described herein are inserted. In another aspect, an mRNA of the disclosure comprises a 5′ UTR comprising the nucleotide sequence set forth in SEQ ID NO: 42 in which a GC-rich RNA element, a C-rich RNA element, and a structural RNA element described herein are inserted.

In another aspect, an mRNA of the disclosure comprises a 5′ UTR comprising the nucleotide sequence set forth in SEQ ID NO: 39 in which a GC-rich RNA element, a C-rich RNA element, and a structural RNA element described herein are inserted.

In one aspect, an mRNA of the disclosure comprises a 5′ UTR comprising the nucleotide sequence set forth in SEQ ID NO: 4 in which a C-rich RNA element and a structural RNA element described herein is inserted. In another aspect, an mRNA of the disclosure comprises a 5′ UTR comprising the nucleotide sequence set forth in SEQ ID NO: 62 in which a C-rich RNA element and a structural RNA element comprising a stem loop described herein is inserted.

In one aspect, an mRNA of the disclosure comprises a 5′ UTR comprising a nucleotide sequence selected from SEQ ID NO: 65, SEQ ID NO: 68, SEQ ID NO: 13, SEQ ID NO: 14, SEQ ID NO: 15, or SEQ ID NO: 16 in which a C-rich RNA element and a structural RNA element described herein is inserted. In another aspect, an mRNA of the disclosure comprises a 5′ UTR comprising the nucleotide sequence set forth in SEQ ID NO: 43 in which a C-rich RNA element and a structural RNA element comprising a stem loop described herein is inserted.

In one aspect, an mRNA of the disclosure comprises a 5′ UTR comprising the nucleotide sequence set forth in SEQ ID NO: 37 in which a GC-rich RNA element and a structural RNA element comprising a stem loop described herein is inserted.

In one aspect, an mRNA of the disclosure comprises a 5′ UTR comprising the nucleotide sequence set forth in SEQ ID NO: 38 in which a GC-rich RNA element and a structural RNA element comprising a stem loop described herein is inserted.

In one aspect, an mRNA of the disclosure comprises a 5′ UTR comprising the nucleotide sequence set forth in SEQ ID NO: 40 in which a GC-rich RNA element and a structural RNA element comprising a stem loop described herein is inserted.

In one aspect, an mRNA of the disclosure comprises a 5′ UTR comprising the nucleotide sequence set forth in SEQ ID NO: 41 in which a GC-rich RNA element and a structural RNA element comprising a stem loop described herein is inserted.

In one aspect, an mRNA of the disclosure comprises a 5′ UTR comprising the nucleotide sequence set forth in SEQ ID NO: 35 in which a structural RNA element comprising a stem loop described herein is inserted.

In one aspect, an mRNA of the disclosure comprises a 5′ UTR comprising the nucleotide sequence set forth in SEQ ID NO: 36 in which a structural RNA element comprising a stem loop described herein is inserted.

In one aspect, an mRNA of the disclosure comprises a 5′ UTR comprising the nucleotide sequence set forth in SEQ ID NO: 44 in which a structural RNA element comprising a stem loop described herein is inserted.

In one aspect, an mRNA of the disclosure comprises a 5′ UTR comprising the nucleotide sequence set forth in SEQ ID NO: 116 in which a C-rich RNA element comprising a stem loop described herein is inserted.

In one aspect, an mRNA of the disclosure comprises a 5′ UTR comprising the nucleotide sequence set forth in SEQ ID NO: 120 in which a C-rich RNA element comprising a stem loop described herein is inserted.

In one aspect, an mRNA of the disclosure comprises a 5′ UTR comprising the nucleotide sequence set forth in SEQ ID NO: 124 in which a C-rich RNA element comprising a stem loop described herein is inserted.

Exemplary 5′ UTRs comprising GC-rich RNA elements, and combinations with structural RNA elements, provided by the disclosure are set forth in Table 8. These 5′ UTRs are useful in the mRNAs of the disclosure.

TABLE 8
Exemplary 5′UTRs with GC-Rich RNA Elements and Structural RNA Elements

| | | SEQ |
|---|---|---|
| 5′ UTRs | Sequence | ID NO |
| F856, | GGGAAATAAGAGAGAAAAGAAGAGTAAGAAGAAATATAAGA | 115 |
| RNAseP_p1 | TCTCCCTGAGCTTCAGGGAGCCCCGGCGCCGCCACC | |
| (DNA) | | |
| F856, | GGGAAAUAAGAGAGAAAAGAAGAGUAAGAAGAAAUAUAAG | 116 |
| RNAseP_p1 | AUCUCCCUGAGCUUCAGGGAGCCCCGGCGCCGCCACC | |
| (RNA) | | |
| RNAseP_p2 | GGGAAATCTCCCTGAGCTTCAGGGAGTAAGAGAGAAAAGA | 119 |
| (DNA) | AGAGTAAGAAGAAATATAAGACCCCGGCGCCGCCACC | |
| RNAseP_p2 | GGGAAAUCUCCCUGAGCUUCAGGGAGUAAGAGAGAAAAGA | 120 |
| (RNA) | AGAGUAAGAAGAAAUAUAAGACCCCGGCGCCGCCACC | |
| RNAseP_p3 | GGGAAATAAGAGAGAAAAGAAGATCTCCCTGAGCTTCAGG | 123 |
| (DNA) | GAGGTAAGAAGAAATATAAGACCCCGGCGCCGCCACC | |
| RNAseP_p3 | GGGAAAUAAGAGAGAAAAGAAGAUCUCCCUGAGCUUCAGG | 124 |
| (RNA) | GAGGUAAGAAGAAAUAUAAGACCCCGGCGCCGCCACC | |

(GC-rich RNA Elements italicized; Structural RNA Elements bold)

### Polynucleotides Comprising Functional RNA Elements in the 3′UTR

In some aspects, the present disclosure provides mRNAs comprising a 3′UTR that comprises a nucleotide sequence that is substantially identical (e.g., about 50%, 60%, 70%, 80%, 90% or about 100% identical) to a 3′UTR of a naturally-occurring mRNA, or a fragment or a variant thereof. In some embodiments, the naturally-occurring mRNA encodes a nuclear encoded mitochondrial protein (NEMP). A 3′UTR comprising a nucleotide sequence that is substantially identical (e.g., about 50%, 60%, 70%, 80%, 90% or about 100% identical) to a 3′UTR of a naturally-occurring mRNA that encodes NEMP, or a fragment or variant thereof, is referred to herein as a “NEMP-derived 3′UTR”.

Mitochondria are sub-cellular organelles that play a central role in many metabolic pathways and are essential for energy production. Nearly all mitochondrial proteins are NEMPs (e.g., mitochondrial proteins encoded by nuclear genes). Most are synthesized on cytosolic ribosomes as precursor polypeptides and are subsequently transported into the mitochondria. NEMPs that are imported into the mitochondria following translation comprise a short N-terminal extensions known as a “mitochondrial targeting sequence” (MTS) that mediates recognition and import of the protein into the mitochondria. Sorting of mRNAs encoding mitochondrial proteins to the mitochondria facilitates the expression and/or functionality of mitochondrial proteins inside the mitochondria.

However, some NEMPs (e.g., Sod2, fumarase) are translated on polysomes bound to the mitochondria and are imported co-translationally (Corral-Debrinski et al., (2000) Mol Cell Biol 20(21):7881-7892; Luk et al., (2005) J Biol Chem 280:22715-22720; Yogev et al., (2007) J Biol Chem 282:29222-29229). Specific signals within the NEMP 3′ untranslated region (UTR) are thought to target the mRNAs to the mitochondria for translation by mitochondria-bound polysomes (Margeout et al (2005) Gene 354:64-71; Corral-Debrinski et al (2000) Mol Cell Biol. 20:7881-7892; Margeot et al (2002) EMBO J 21:6893-6904). While in no way bound by theory, signals in NEMP-derived 3′UTRs that mediate import to the mitochondria are thought to comprise RNA elements, for example an RNA element that is a specific sequence of the 3′UTR or specific structural element of the 3′UTR. Such RNA elements are thought improve mRNA expression level and activity of encoded protein by regulating the stabilization, localization and/or translation of an mRNA comprising the NEMP-derived 3′UTR.

In some embodiments, an mRNA of the disclosure comprises a NEMP-derived 3′UTR wherein the 3′UTR comprises one or more RNA elements that regulates the stabilization of an mRNA. In some embodiments, an RNA element of the NEMP-derived 3′UTR binds to one or more RNA-binding proteins, wherein binding of a 3′UTR to one or more RNA-binding proteins promotes the stabilization, localization, or translation of an mRNA comprising the NEMP-derived 3′UTR. In some embodiments, an RNA element of the NEMP-derived 3′UTR blocks an interaction with one or more RNA-binding proteins, wherein blocking an interaction of the 3′UTR with one or more RNA-binding proteins promotes the stabilization, localization, or translation of an mRNA comprising the NEMP-derived 3′UTR.

In some embodiments, an mRNA of the disclosure comprises a 3′ UTR derived from an mRNA encoding a NEMP, wherein the 3′UTR comprises one or more RNA elements that regulates the localization of the mRNA. In a non-limiting example, the one or more RNA elements binds to an RNA-binding protein of the cytoskeleton, thereby mediating trafficking of the mRNA to a subcellular location. In another non-limiting example, the one or more RNA elements binds to an RNA-binding protein that is a cellular membrane protein, thereby mediating retention of the mRNA at a subcellular location.

In some embodiments, an mRNA of the disclosure comprises a 3′ UTR derived from an mRNA encoding a NEMP, wherein the 3′UTR comprises one or more RNA elements that regulates the translation of the mRNA. In a non-limiting example, the one or more RNA elements binds to an RNA-binding protein of the translational machinery (e.g., a 43S pre-initiation complex, a ribosome, a polysome), thereby facilitating translation of the mRNA.

In some embodiments, an mRNA of the disclosure comprises a 3′ UTR derived from an mRNA encoding a NEMP, wherein the 3′UTR comprises one or more RNA elements that regulates the stability of the mRNA. In a non-limiting example, the one or more RNA elements binds to an RNA-binding protein that prevents mRNA degradation, thereby increasing stability of the mRNA transcript. In another non-limiting example, the one or more RNA elements blocks an interaction with an RNA-binding protein that functions in a pathway to promote mRNA degradation, thereby increasing stability of the mRNA transcript.

In some embodiments, an mRNA of the disclosure comprises a 3′ UTR derived from an mRNA encoding a NEMP, wherein the 3′UTR comprises one or more RNA elements that binds to an RNA-binding protein of known function (e.g., identified by a public database such as one described by Berglund et al. (2008) Nucleic Acids Res 36:D263-266). In some embodiments, the RNA-binding protein is known to promote mRNA localization (e.g., a cytoskeletal RNA-binding protein, a membrane-bound RNA-binding protein). In some embodiments, the RNA-binding protein is known to promote mRNA stability. In some embodiments, the RNA-binding protein is known to promote mRNA translation (e.g., a RNA-binding protein of the translational machinery). Methods of identifying interactions between an RNA element and an RNA-binding protein are known in the art. In some embodiments, a method of identifying an interaction comprises first immobilizing a polynucleotide (e.g., an RNA, an mRNA, an mRNA UTR) comprising the RNA element and subsequently incubating the immobilized polynucleotide with cellular extracts. Following incubation, proteins bound to the immobilized polynucleotide are characterized by a method of quantitative mass-spectrometry as described by Butter, et al (2009) Proc Natl Acad Sci USA 106:10626-10631 and Tsvetanova, et al (2010) PLos ONE 5:e12671, incorporated herein by reference. In some embodiments, a method of identifying an interaction comprises preparation of an array of RNA-binding proteins and subsequently incubating the array with a fluorescently tagged polynucleotide (e.g., an RNA, an mRNA, an mRNA UTR). The fluorescent intensity of each individual protein spot is used to quantify binding affinity of each protein in the array for the fluorescently tagged polynucleotide as described by Scherrer, et al (2010) PLoS ONE 5:e15499, incorporated herein by reference.

In some embodiments, an mRNA of the disclosure comprises a 3′ UTR derived from an mRNA encoding a NEMP that regulates the localization of the mRNA, whereby regulation of mRNA localization increases or enhances expression and/or activity of a protein encoded by the mRNA. In some embodiments, an mRNA of the disclosure comprises a 3′ UTR derived from an mRNA encoding a NEMP that promotes stability of the mRNA, whereby increased mRNA stability increases or enhances expression and/or activity of a protein encoded by the mRNA. In some embodiments, an mRNA of the disclosure comprises a 3′ UTR derived from an mRNA encoding a NEMP that promotes translation of the mRNA, whereby increased mRNA translation increases or enhances expression and/or activity of a protein encoded by the mRNA.

In some embodiments, an mRNA of the disclosure comprises a 3′ UTR wherein the nucleotide sequence of the 3′UTR is substantially identical to the nucleotide sequence of a 3′ UTR derived from an mRNA encoding a NEMP. In some embodiments, an mRNA of the disclosure comprises a 3′ UTR wherein the nucleotide sequence of the 3′UTR is at least 50%, at least 60%, at least 70%, at least 80%, at least 85%, at least 90%, at least 95%, at least 96%, at least 97%, at least 98%, at least 99%, or 100% identical to the nucleotide sequence of a 3′ UTR derived from an mRNA encoding a NEMP.

In some embodiments, an mRNA of the disclosure comprises a 3′ UTR derived from a naturally-occurring mRNA encoding a NEMP, wherein the 3′UTR differs from the naturally-occurring 3′UTR by one or more nucleotide substitutions. In some embodiments, an mRNA of the disclosure comprises a 3′ UTR derived from a naturally-occurring mRNA encoding a NEMP, wherein the 3′UTR differs from the naturally-occurring 3′UTR by 1, 2, 3, 4, 5, 6, 7, 8, 9, 10, 11, 12, 13, 14, 15, 16, 17, 18, 19, or 20 or more nucleotides. In some embodiments, an mRNA of the disclosure comprises a 3′ UTR derived from a naturally-occurring mRNA encoding a NEMP, wherein the 3′UTR differs from the naturally-occurring 3′UTR by 1-5, 5-10, 10-15, 15-20, 20-25, 25-30, 30-35, 35-40, 40-45, 45-50 or about 50 or more nucleotides.

In some embodiments, an mRNA of the disclosure comprises a 3′ UTR derived from an mRNA encoding a NEMP, wherein the 3′UTR is about 50 nucleotides, about 100 nucleotides, about 150 nucleotides, about 200 nucleotides, about 300 nucleotides, about 500 nucleotides, about 1000 nucleotides, about 1500 nucleotides in length. In some embodiments, the NEMP-derived 3′ UTR is about 100-200 nucleotides in length. In some embodiments, the NEMP-derived 3′ UTR is about 100 nucleotides, about 105 nucleotides, about 110 nucleotides, about 115 nucleotides, about 120 nucleotides, about 125 nucleotides, about 130 nucleotides, about 135 nucleotides, about 140 nucleotides, about 145 nucleotides, about 150 nucleotides, about 155 nucleotides, about 160 nucleotides, about 165 nucleotides, about 170 nucleotides, about 175 nucleotides, about 180 nucleotides, about 185 nucleotides, about 190 nucleotides, about 195 nucleotides, or about 200 nucleotides in length. In some embodiments, the NEMP-derived 3′ UTR is about 200-400 nucleotides in length. In some embodiments, the NEMP-derived 3′ UTR is about 200 nucleotides, about 220 nucleotides, about 240 nucleotides, about 260 nucleotides, about 280 nucleotides, about 300 nucleotides, about 320 nucleotides, about 340 nucleotides, about 360 nucleotides, about 380 nucleotides, or about 400 nucleotides in length. In some embodiments, the mitochondrial targeting 3′ UTR is about 400-1000 nucleotides in length. In some embodiments, the mitochondrial targeting 3′ UTR is about 400 nucleotides, about 500 nucleotides, about 600 nucleotides, about 700 nucleotides, about 800 nucleotides, about 900 nucleotides, or about 1000 nucleotides in length. In some embodiments, the NEMP-derived 3′ UTR is about 1000-1500 nucleotides in length. In some embodiments, the NEMP-derived 3′ UTR is about 1100 nucleotides, about 1200 nucleotides, about 1300 nucleotides, about 1400 nucleotides, or about 1500 nucleotides in length. In some embodiments, the NEMP-derived 3′ UTR is about 138 nucleotides in length. In some embodiments, the NEMP-derived 3′ UTR is about 166 nucleotides in length. In some embodiments, the NEMP-derived 3′ UTR is about 167 nucleotides in length. In some embodiments, the NEMP-derived 3′ UTR is about 233 nucleotides in length. In some embodiments, the NEMP-derived 3′ UTR is about 371 nucleotides in length. In some embodiments the NEMP-derived 3′ UTR is about 1155 nucleotides in length. In some embodiments, the NEMP-derived 3′ UTR is about 1371 nucleotides in length.

In some embodiments, an mRNA of the disclosure comprises a 3′ UTR derived from an mRNA encoding a NEMP, wherein the 3′UTR is encoded by a gene encoding a NEMP and wherein the gene is selected from the group consisting of: human OXAL1, human MRPS12, and mouse Sod2.

In some embodiments, an mRNA of the disclosure comprises a 3′ UTR comprising a nucleotide sequence that is substantially identical to a 3′UTR of an mRNA encoding a NEMP, or a fragment or variant thereof, wherein the 3′UTR comprises a nucleotide sequence at least 50%, at least 60%, at least 70%, at least 80%, at least 85%, at least 90%, at least 95%, at least 96%, at least 97%, at least 98%, at least 99%, or 100% identical to a nucleotide sequence selected from the group consisting of: SEQ ID NO: 72, SEQ ID NO: 74, SEQ ID NO: 76, SEQ ID NO: 78.

In some embodiments, an mRNA of the disclosure comprises a 3′ UTR derived from an mRNA encoding a NEMP, wherein the 3′UTR comprises a nucleotide sequence at least 50%, at least 60%, at least 70%, at least 80%, at least 85%, at least 90%, at least 95%, at least 96%, at least 97%, at least 98%, at least 99%, or 100% identical to the nucleotide sequence of SEQ ID NO: 72.

In some embodiments, an mRNA of the disclosure comprises a 3′ UTR derived from an mRNA encoding a NEMP, wherein the 3′UTR comprises a nucleotide sequence at least 50%, at least 60%, at least 70%, at least 80%, at least 85%, at least 90%, at least 95%, at least 96%, at least 97%, at least 98%, at least 99%, or 100% identical to the nucleotide sequence of SEQ ID NO: 74.

In some embodiments, an mRNA of the disclosure comprises a 3′ UTR derived from an mRNA encoding a NEMP, wherein the 3′UTR comprises a nucleotide sequence at least 50%, at least 60%, at least 70%, at least 80%, at least 85%, at least 90%, at least 95%, at least 96%, at least 97%, at least 98%, at least 99%, or 100% identical to the nucleotide sequence of SEQ ID NO: 76.

In some embodiments, an mRNA of the disclosure comprises a 3′ UTR derived from an mRNA encoding a NEMP, wherein the 3′UTR comprises a nucleotide sequence at least 50%, at least 60%, at least 70%, at least 80%, at least 85%, at least 90%, at least 95%, at least 96%, at least 97%, at least 98%, at least 99%, or 100% identical to the nucleotide sequence of SEQ ID NO: 78.

In some embodiments, an mRNA of the disclosure comprises a 3′ UTR derived from an mRNA encoding a NEMP, wherein the 3′ UTR comprises one or more miRNA binding sites, such as those described herein. In some embodiments, the miRNA binding site binds to miR-142-3p or miR-142-5p.

In some embodiments, the miRNA binding site that binds to miR-142-3p comprises a nucleotide sequence at least 50%, at least 60%, at least 70%, at least 80%, at least 85%, at least 90%, at least 95%, at least 96%, at least 97%, at least 98%, at least 99%, or 100% identical to the nucleotide sequence of SEQ ID NO: 179. In some embodiments, the miRNA binding site that binds to miR-142-3p comprises a nucleotide sequence at least 50%, at least 60%, at least 70%, at least 80%, at least 85%, at least 90%, at least 95%, at least 96%, at least 97%, at least 98%, at least 99%, or 100% identical to the nucleotide sequence of SEQ ID NO: 181.

In some embodiments, an mRNA of the disclosure comprises a 3′UTR derived from an mRNA encoding a NEMP, or a fragment or variant thereof, wherein the 3′UTR comprises one or more miRNA binding sites at any location in the 3′UTR. In some embodiments, the one or more miRNA binding sites are located proximal to the one or more stop codons at the 5′end of the 3′UTR. In some embodiments, the one or more miRNA binding sites are located downstream of and immediately adjacent to the one or more stop codons at the 5′end of the 3′UTR. In some embodiments, the one or more miRNA binding sites are located about 45-50, about 40-45, about 35-40, about 30-35, about 25-30, about 20-25, about 15-20, about 10-15, about 6-10 nucleotides, about 1-5 nucleotide(s), or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) downstream of the one or more stop codons at the 5′end of the 3′UTR. In some embodiments, the one or more miRNA binding sites are located about 50, about 45, about 40, about 35, about 30, about 25, about 20, about 15, about 10 or about 5, about 4, about 3, about 2, or about 1 nucleotide(s) downstream of the one or more stop codons at the 5′end of the 3′UTR. In some embodiments, the one or more miRNA binding sites are located about 5, about 4, about 3, about 2, or about 1 nucleotide(s) downstream of the one or more stop codons at the 5′end of the 3′UTR. In some embodiments, the one or more miRNA binding sites are located 5 nucleotides downstream of the one or more stop codons at the 5′end of the 3′UTR. In some embodiments, the one or more miRNA binding sites are located 6 nucleotides downstream of the one or more stop codons at the 5′end of the 3′UTR. In some embodiments, the one or more miRNA binding sites are located 7 nucleotides downstream of the one or more stop codons at the 5′end of the 3′UTR. In some embodiments, the one or more miRNA binding sites are located 8 nucleotides downstream of the one or more stop codons at the 5′end of the 3′UTR. In some embodiments, the one or more miRNA binding sites are located 9 nucleotides downstream of the one or more stop codons at the 5′end of the 3′UTR. In some embodiments, the one or more miRNA binding sites are located 10 nucleotides downstream of the one or more stop codons at the 5′end of the 3′UTR.

In some embodiments, the one or more miRNA binding sites are located proximal the 3′end of the 3′UTR. In some embodiments, the one or more miRNA binding sites are located upstream of and immediately adjacent to the 3′end of the 3′UTR. In some embodiments, the one or more miRNA binding sites are located about 1-5, about 6-10, about 10-15, about 15-20, about 20-25, about 25-30, about 30-35, about 35-40, about 40-45, or about 45-50 nucleotide(s) or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotide(s) upstream of the 3′end of the 3′UTR. In some embodiments, the one or more miRNA binding sites are located about 1, about 2, about 3, about 4, about 5, about 10, about 15, about 20, about 25, about 30, about 35, about 40, about 45, or about 50 nucleotide(s) upstream of the 3′end of the 3′UTR. In some embodiments, the one or more miRNA binding sites are located about 1, about 2, about 3, about 4, or about 5 nucleotide(s) upstream of the 3′end of the 3′UTR. In some embodiments, the one or more miRNA binding sites are located 6 nucleotides upstream of the 3′end of the 3′UTR. In some embodiments, the one or more miRNA binding sites are located 7 nucleotides upstream of the 3′end of the 3′UTR. In some embodiments, the one or more miRNA binding sites are located 8 nucleotides upstream of the 3′end of the 3′UTR. In some embodiments, the one or more miRNA binding sites are located 9 nucleotides upstream of the 3′end of the 3′UTR. In some embodiments, the one or more miRNA binding sites are located 10 nucleotides upstream of the 3′end of the 3′UTR.

In some embodiments, an mRNA of the disclosure comprises a 3′UTR derived from an mRNA encoding a NEMP, or a fragment or variant thereof, wherein the 3′UTR comprises 1, 2, 3, 4, 5, 6, 7, 8, 9, 10 or more miRNA binding site(s). In some embodiments, the NEMP-derived 3′UTR comprises one miRNA binding site. In some embodiments, the NEMP-derived 3′UTR comprises two miRNA binding sites. In some embodiments, the NEMP-derived 3′UTR comprises three miRNA binding sites. In some embodiments, the NEMP-derived 3′UTR comprises four miRNA binding sites. In some embodiments, the NEMP-derived 3′UTR comprises five miRNA binding sites. In some embodiments, the NEMP-derived 3′UTR comprises six miRNA binding sites. In some embodiments, the NEMP-derived 3′UTR comprises seven miRNA binding sites. In some embodiments, the NEMP-derived 3′UTR comprises eight miRNA binding sites. In some embodiments, the NEMP-derived 3′UTR comprises nine miRNA binding sites. In some embodiments, the NEMP-derived 3′UTR comprises ten miRNA binding sites.

In some embodiments, an mRNA of the disclosure comprises a 3′UTR derived from an mRNA encoding a NEMP, or a fragment or variant thereof, wherein the 3′UTR comprises one or more (e.g., 1, 2, 3, 4, 5, 6, 7, 8, 9, or 10) miRNA binding sites that bind to a miRNA selected from a group consisting of: miR-142-3p, miR-142-5p, miR-122-3p, or miR-122-5p. In some embodiments, the one or more (e.g., 1, 2, 3, 4, 5, 6, 7, 8, 9, or 10) miRNA binding site(s) bind to miR-142-3p. In some embodiments, the 3′UTR comprises one miRNA binding site that binds to miR-142-3p. In some embodiments, the 3′UTR comprises two miRNA binding sites that bind to miR-142-3p. In some embodiments, the 3′UTR comprises three miRNA binding sites that bind to miR-142-3p. In some embodiments, the 3′UTR comprises four miRNA binding sites that bind to miR-142-3p. In some embodiments, the 3′UTR comprises five miRNA binding sites that bind to miR-142-3p. In some embodiments, the 3′UTR comprises six miRNA binding sites that bind to miR-142-3p. In some embodiments, the 3′UTR comprises seven miRNA binding sites that bind to miR-142-3p. In some embodiments, the 3′UTR comprises eight miRNA binding sites that bind to miR-142-3p. In some embodiments, the 3′UTR comprises nine miRNA binding sites that bind to miR-142-3p. In some embodiments, the 3′UTR comprises ten miRNA binding sites that bind to miR-142-3p.

In some embodiments, an mRNA of the disclosure comprises a 3′UTR derived from an mRNA encoding a NEMP, or a fragment or variant thereof, wherein the 3′UTR comprises more than one (e.g., 2, 3, 4, 5, 6, 7, 8, 9, or 10) miRNA binding sites, wherein an upstream miRNA binding site is located directly adjacent to one or more downstream miRNA binding site(s). In some embodiments, the more than one (e.g., 2, 3, 4, 5, 6, 7, 8, 9, or 10) miRNA binding sites comprise intervening nucleotides. In some embodiments, an upstream miRNA binding site is separate from a downstream miRNA binding site by about 1-5, about 1-10, about 5-10, about 5-15, about 10-20, about 15-20, about 15-30, or about 20-30 nucleotide(s) or about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotide(s). In some embodiments, an upstream miRNA binding site is separate from a downstream miRNA binding site by about 1, about 2, about 3, about 4, about 5, about 10, about 15, about 20, about 25, or about 30 nucleotide(s). In some embodiments, an upstream miRNA binding site is separate from a downstream miRNA binding site by about 3 nucleotides. In some embodiments, an upstream miRNA binding site is separate from a downstream miRNA binding site by about 4 nucleotides. In some embodiments, an upstream miRNA binding site is separate from a downstream miRNA binding site by about 5 nucleotides. In some embodiments, an upstream miRNA binding site is separate from a downstream miRNA binding site by about 6 nucleotides. In some embodiments, an upstream miRNA binding site is separate from a downstream miRNA binding site by about 7 nucleotides. In some embodiments, an upstream miRNA binding site is separate from a downstream miRNA binding site by about 8 nucleotides. In some embodiments, an upstream miRNA binding site is separate from a downstream miRNA binding site by about 9 nucleotides. In some embodiments, an upstream miRNA binding site is separate from a downstream miRNA binding site by about 10 nucleotides.

In some embodiments, an mRNA of the disclosure comprises a 3′ UTR derived from an mRNA encoding a NEMP, or a fragment or variant thereof, wherein the 3′UTR comprises a nucleotide sequence at least 50%, at least 60%, at least 70%, at least 80%, at least 85%, at least 90%, at least 95%, at least 96%, at least 97%, at least 98%, at least 99%, or 100% identical to the nucleotide sequence of SEQ ID NO: 78 and wherein the 3′UTR comprises one or more miRNA binding sites (e.g., a miR-142-3p binding site). In some embodiments, the 3′UTR comprises one or more miRNA binding sites (e.g., a miR-142-3p binding site) proximal to the 3′end, wherein the 3′UTR comprises the nucleotide sequence of SEQ ID NO: 170. In some embodiments, the 3′UTR comprises one or more miRNA binding sites (e.g., a miR-142-3p binding site) proximal to one or more stop codons at the 5′end, wherein the 3′UTR comprises the nucleotide sequence of SEQ ID NO: 172.

In some embodiments, an mRNA of the disclosure comprises a 3′ UTR derived from an mRNA encoding a NEMP, or a fragment or variant thereof, wherein the 3′UTR comprises a nucleotide sequence at least 50%, at least 60%, at least 70%, at least 80%, at least 85%, at least 90%, at least 95%, at least 96%, at least 97%, at least 98%, at least 99%, or 100% identical to the nucleotide sequence of SEQ ID NO: 76 and wherein the 3′UTR comprises one or more miRNA binding sites (e.g., a miR-142-3p binding site). In some embodiments, the 3′UTR comprises one or more miRNA binding sites (e.g., a miR-142-3p binding site) proximal to the 3′end, wherein the 3′UTR comprises the nucleotide sequence of SEQ ID NO: 174. In some embodiments, the 3′UTR comprises one or more miRNA binding sites (e.g., a miR-142-3p binding site) proximal to one or more stop codons at the 5′end, wherein the 3′UTR comprises the nucleotide sequence of SEQ ID NO: 176.

In some embodiments, the NEMP-derived 3′ UTR comprises one or more (e.g., 1, 2, 3 or 4) different modified nucleobases, nucleosides, or nucleotides, such as those described herein.

In some embodiments, an mRNA of the disclosure comprises a 3′ UTR derived from an mRNA encoding a NEMP, wherein expression of a polypeptide encoded by the mRNA is increased relative to an equivalent mRNA comprising a 3′UTR with a nucleotide sequence identified by SEQ ID NO: 150 or SEQ ID NO: 70. In some embodiments, the expression level is increased by at least about 1-fold, about 2-fold, about 3-fold, about 4-fold, about 5-fold, about 6-fold, about 7-fold, about 8-fold, about 9-fold, about 10-fold or more. In some embodiments, activity is increased by about 1%, about 2%, about 3%, about 4%, about 5%, about 6%, about 7%, about 8%, about 9%, about 10%, about 15% about 20%, about 30%, about 40%, about 50%, about 60%, about 70%, about 80%, about 90%, or about 100%.

In some embodiments, an mRNA of the disclosure comprises a 3′ UTR derived from an mRNA encoding a NEMP, wherein activity of a polypeptide encoded by the mRNA is increased relative to an equivalent mRNA comprising a 3′UTR with a nucleotide sequence identified by SEQ ID NO: 150 or SEQ ID NO: 70. In some embodiments, activity is increased by at least about 1-fold, about 2-fold, about 3-fold, about 4-fold, about 5-fold, about 6-fold, about 7-fold, about 8-fold, about 9-fold, about 10-fold or more. In some embodiments, activity is increased by about 1%, about 2%, about 3%, about 4%, about 5%, about 6%, about 7%, about 8%, about 9%, about 10%, about 15% about 20%, about 30%, about 40%, about 50%, about 60%, about 70%, about 80%, about 90%, or about 100%.

Exemplary mRNAs Comprising Functional RNA Elements

In some embodiments, the disclosure provides an mRNA comprising one or more mitochondrial targeting elements, wherein the mRNA comprises a 5′ untranslated region (5′UTR), an open reading frame (ORF) encoding a polypeptide of interest, and a 3′UTR, wherein the 5′UTR comprises one or more RNA elements of the disclosure and/or wherein the 3′UTR comprises the nucleotide sequence of a 3′UTR of a naturally-occurring mRNA encoding a nuclear encoded mitochondrial protein (NEMP), or a fragment or variant thereof. In some embodiments, the 5′UTR comprises one or more structural RNA elements comprising a stem-loop (e.g., an RNAse P stem loop). In some embodiments, the 3′ UTR comprises the nucleotide sequence of a 3′UTR derived from a naturally-occurring mRNA encoding a NEMP, or a fragment or variant thereof. In some embodiments, the 5′UTR comprises one or more structural RNA elements comprising a stem-loop (e.g., an RNAse P stem loop), and the 3′UTR comprises the nucleotide sequence of a 3′UTR derived from a naturally-occurring mRNA encoding a NEMP, or a fragment or variant thereof.

In some aspects, the disclosure provides an mRNA comprising one or more RNA elements, wherein the mRNA comprises:
(i) a 5′ UTR comprising SEQ ID NO: 45
(ii) an ORF encoding a polypeptide of interest; and
(iii) a 3′UTR,
wherein the 5′UTR comprises a structural RNA element comprising a stem-loop comprising a nucleotide sequence selected from the group consisting of: SEQ ID NO: 6 or SEQ ID NO: 47, and/or wherein the 3′UTR comprises the nucleotide sequence of a 3′UTR derived from a naturally-occurring mRNA encoding a NEMP, or a fragment or variant thereof.

In some aspects, the disclosure provides an mRNA comprising one or more RNA elements, wherein the mRNA comprises:
(i) a 5′ UTR comprising SEQ ID NO: 4;
(ii) an ORF encoding a polypeptide of interest; and
(iii) a 3′UTR,
wherein the 5′UTR comprises a structural RNA element comprising a stem-loop comprising a nucleotide sequence selected from the group consisting of: SEQ ID NO: 6 or SEQ ID NO: 47, and/or wherein the 3′UTR comprises the nucleotide sequence of a 3′UTR derived from a naturally-occurring mRNA encoding a NEMP, or a fragment or variant thereof. In some embodiments, the structural RNA element comprises the nucleotide sequence of SEQ ID NO: 6. In some embodiments, the structural RNA element comprises the nucleotide sequence of SEQ ID NO: 47. In some embodiments, the 5′UTR comprises the nucleotide sequence of SEQ ID NO: 116. In some embodiments, the 5′UTR comprises the nucleotide sequence of SEQ ID NO: 120. In some embodiments, the 5′UTR comprises the nucleotide sequence of SEQ ID NO: 124.

In some aspects, the disclosure provides an mRNA comprising one or more RNA elements, wherein the mRNA comprises:
(i) a 5′ UTR comprising SEQ ID NO: 35 or SEQ ID NO: 36;
(ii) an ORF encoding a polypeptide of interest; and
(iii) a 3′UTR,
wherein the 5′UTR comprises a structural RNA element comprising a stem-loop comprising a nucleotide sequence selected from the group consisting of: SEQ ID NO: 6 or SEQ ID NO: 47, and/or wherein the 3′UTR comprises the nucleotide sequence of a 3′UTR derived from a naturally-occurring mRNA encoding a NEMP, or a fragment or variant thereof. In some embodiments, the structural RNA element comprises the nucleotide sequence of SEQ ID NO: 6. In some embodiments, the structural RNA element comprises the nucleotide sequence of SEQ ID NO: 47. In some embodiments, the 5′ UTR comprises a nucleotide sequence selected from the group consisting of SEQ ID NO: 128, SEQ ID NO: 132, SEQ ID NO: 136, SEQ ID NO: 140, and SEQ ID NO: 144. In some embodiments, the 5′ UTR comprises the nucleotide sequence of SEQ ID NO: 128. In some embodiments, the 5′ UTR comprises the nucleotide sequence of SEQ ID NO: 132. In some embodiments, the 5′ UTR comprises the nucleotide sequence of SEQ ID NO: 136. In some embodiments, the 5′ UTR comprises the nucleotide sequence of SEQ ID NO: 140. In some embodiments, the 5′ UTR comprises the nucleotide sequence of SEQ ID NO: 144.

In some embodiments the polypeptide is a NEMP, optionally wherein the NEMP is heterologous to the 3′UTR.

In some embodiments, the 3′UTR comprises a nucleotide sequence selected from the group consisting of: SEQ ID NO: 72, SEQ ID NO: 74, SEQ ID NO: 76, SEQ ID NO: 78. In some embodiments, the 3′ UTR comprises the nucleotide sequence of SEQ ID: 78.

In some embodiments, the disclosure provides an mRNA comprising one or more RNA elements, wherein the mRNA comprises:
(i) a 5′ UTR comprising a structural RNA element comprising a stem-loop;
(ii) an ORF encoding a polypeptide of interest; and
(iii) a 3′UTR comprising the nucleotide sequence of a 3′UTR derived from a naturally-occurring mRNA encoding a NEMP, or a fragment or variant thereof, wherein the 3′UTR comprises a nucleotide sequence selected from the group consisting of: SEQ ID NO: 72, SEQ ID NO: 74, SEQ ID NO: 76, SEQ ID NO: 78. In some embodiments, the 3′UTR comprises the nucleotide sequence of SEQ ID: 78. In some embodiments, the structural RNA element comprising a stem-loop comprises a nucleotide sequence selected from the group consisting of: SEQ ID NO: 6 or SEQ ID NO: 47. In some embodiments, the structural RNA element comprising a stem-loop comprises the nucleotide sequence of SEQ ID NO: 6. In some embodiments, the structural RNA element comprising a stem-loop comprises the nucleotide sequence of SEQ ID NO: 47.

In some aspects, the disclosure provides an mRNA comprising one or more RNA elements, wherein the mRNA comprises:
(i) a 5′ UTR comprising SEQ ID NO: 4;
(ii) an ORF encoding a polypeptide of interest; and
(iii) a 3′UTR comprising SEQ ID NO: 78,

Wherein the 5′UTR comprises a structural RNA element comprising a stem-loop comprising a nucleotide sequence selected from the group consisting of: SEQ ID NO: 6 or SEQ ID NO: 47 and/or the 3′UTR comprises one or more microRNA (miR) binding sites (e.g., a miR-142-3p binding site). In some embodiments, the structural RNA element comprises the nucleotide sequence of SEQ ID NO: 6.

In some embodiments, the structural RNA element comprises the nucleotide sequence of SEQ ID NO: 47. In some embodiments, the 5′UTR comprises the nucleotide sequence of SEQ ID NO: 120. In some embodiments, the 3′UTR comprises one or more miR binding site(s) that comprises a nucleotide sequence identified by SEQ ID NO: 179. In some embodiments, the 3′UTR comprises the nucleotide sequence of SEQ ID NO: 170. In some embodiments, the 3′UTR comprises the nucleotide sequence of SEQ ID NO: 172.

In some aspects, the disclosure provides an mRNA comprising one or more RNA elements, wherein the mRNA comprises:
(i) a 5′ UTR comprising SEQ ID NO: 4;
(ii) an ORF encoding a polypeptide of interest; and
(iii) a 3′UTR comprising SEQ ID NO: 76,

Wherein the 5′UTR comprises a structural RNA element comprising a stem-loop comprising a nucleotide sequence selected from the group consisting of: SEQ ID NO: 6 or SEQ ID NO: 47 and/or the 3′UTR comprises one or more microRNA (miR) binding sites (e.g., a miR-142-3p binding site). In some embodiments, the structural RNA element comprises the nucleotide sequence of SEQ ID NO: 6.

In some embodiments, the structural RNA element comprises the nucleotide sequence of SEQ ID NO: 47. In some embodiments, the 5′UTR comprises the nucleotide sequence of SEQ ID NO: 120. In some embodiments, the 3′UTR comprises one or more miR binding site(s) that comprises a nucleotide sequence identified by SEQ ID NO: 179. In some embodiments, the 3′UTR comprises the nucleotide sequence of SEQ ID NO: 174. In some embodiments, the 3′UTR comprises the nucleotide sequence of SEQ ID NO: 176.

In some embodiments, the mRNAs provided by the disclosure comprise at least one chemically modified nucleoside. In some embodiments, the at least one chemically modified nucleoside is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine. In some embodiments, least about 10%, at least about 20%, at least about 30%, at least about 40%, at least about 50%, at least about 60%, at least about 70%, at least about 80%, at least about 90%, at least about 95%, at least about 99%, or about 100% of the nucleosides comprising the mRNA comprise the at least one chemically modified nucleoside.

In some embodiments, the at least one chemically modified nucleoside is N1-methylpseudouridine, and wherein at least 80%, at least 85%, at least 90%, at least 95%, at least 99%, or 100% of the uracil nucleotides are N1-methylpseudouridine. In some embodiments, the mRNA is fully modified with N1-methylpseudouridine. In some embodiments, the at least one modified nucleoside is 5-methoxyuridine. In some embodiments, at least 95% of uracil nucleotides comprising the ORF comprise 5-methoxyuridine, and wherein the uracil content in the ORF is between about 100% and about 150% of the theoretical minimum.

In some embodiments, the uracil content in the ORF is between about 110% and about 150%, about 115% and about 150%, about 120% and about 150%, about 110% and about 145%, about 115% and about 145%, about 120% and about 145%, about 110% and about 140%, about 115% and about 140%, or about 120% and about 140% of the theoretical minimum.

In some embodiments, the uracil content in the ORF is between (i) 115%, 116%, 117%, 118%, 119%, 120%, 121%, 122%, or 123% and (ii) 138%, 139%, 140%, 141%, 142%, 143%, 144%, or 145%.

In some embodiments, at least 99% of uracil nucleotides comprising the ORF comprise 5-methoxyuridine. In some embodiments, 100% of uracil nucleotides comprising the ORF comprise 5-methoxyuridines. In some embodiments, the mRNA is fully modified with 5-methoxyuridine.

In some embodiments, the disclosure provides an mRNA comprising:
(i) a5′UTR;
(ii) an ORF encoding a polypeptide of interest; and
(iii) a 3′UTR,
wherein the 5′ UTR comprises a structural RNA element comprising a stem-loop, and/or wherein the 3′ UTR comprises the nucleotide sequence of a 3′UTR derived from a naturally-occurring mRNA encoding a NEMP, or a fragment or variant thereof. wherein the mRNA is fully modified with N1-methylpseudouridine.

In some embodiments, the disclosure provides an mRNA comprising:
(i) a5′UTR;
(ii) an ORF encoding a polypeptide of interest; and
(iii) a 3′UTR,
wherein the 5′ UTR comprises a structural RNA element comprising a stem-loop, and/or wherein the 3′ UTR comprises the nucleotide sequence of a 3′UTR derived from a naturally-occurring mRNA encoding a NEMP, or a fragment or variant thereof, wherein at least 95% of uracil nucleotides comprising the ORF comprise 5-methoxyuridine, and wherein the uracil content in the ORF is between about 100% and about 150% of the theoretical minimum. In some embodiments, the uracil content in the ORF is between about 110% and about 150%, about 115% and about 150%, about 120% and about 150%, about 110% and about 145%, about 115% and about 145%, about 120% and about 145%, about 110% and about 140%, about 115% and about 140%, or about 120% and about 140% of the theoretical minimum. In some embodiments, the uracil content in the ORF is between (i) 115%, 116%, 117%, 118%, 119%, 120%, 121%, 122%, or 123% and (ii) 138%, 139%, 140%, 141%, 142%, 143%, 144%, or 145% of the theoretical minimum. In some embodiments, at least 99% of uracil nucleotides comprising the ORF comprise 5-methoxyuridine. In some embodiments, 100% of uracil nucleotides comprising the ORF comprise 5-methoxyuridines. In some embodiments, the mRNA is fully modified with 5-methoxyuridine.

In some embodiments, the mRNA comprises a poly A tail (e.g., a poly A tail of about 100 nucleotides). In some embodiments, the mRNA comprises a 5′ Cap 1 structure.

In some embodiments, an expression level and an activity of the polypeptide translated from the mRNA is increased relative to an mRNA that does not comprise the 3′ UTR or that comprises a reference 3′ UTR.

In some embodiments, an expression level and/or an activity of the polypeptide translated from the mRNA is increased relative to an mRNA that does not comprise the one or more RNA elements.

In some embodiments, the increase in an expression level and/or an activity of the polypeptide translated from the mRNA is additive or synergistic.

In some embodiments, the ORF comprises a nucleotide sequence encoding a mitochondrial protein described in the MitoCarta2.0 mitochondria protein inventory.

Methods of Measuring mRNA Functionality

In some aspects, the disclosure provides mRNAs comprising a NEMP-derived 3′UTR, a 5′UTR comprising one or more functional RNA elements, or a combination thereof wherein the 5′UTR, the 3′UTR, or both promote, increase, or activate mRNA stability, cellular localization and/or a desired translational regulatory activity when compared to an equivalent mRNA comprising a reference 3′UTR, a reference 5′UTR, or a combination thereof. In some embodiments, the NEMP-derived 3′UTR that promotes, increases, or activates mRNA stability, cellular localization and/or a desired translational regulatory activity is referred to as the “test 3′UTR”. In some embodiments, the 5′UTR that promotes, increases, or activates mRNA stability, cellular localization and/or a desired translational regulatory activity is referred to as the “test 5′UTR”.

In some embodiments, a reference 3′UTR comprises a nucleotide sequence identified by SEQ ID NO: 150 or SEQ ID NO: 70. In some embodiments, a reference 3′UTR comprises a nucleotide sequence that is at least 50%, at least 60%, at least 70%, at least 80%, at least 85%, at least 90%, at least 95%, at least 96%, at least 97%, at least 98%, at least 99%, or 100% identical to the nucleotide sequence identified by SEQ ID NO: 150 or SEQ ID NO: 70. In some embodiments, a reference 3′UTR comprises a nucleotide sequence identical or equivalent to the nucleotide sequence of the test 3′UTR, wherein one or more functional RNA elements of the test 3′UTR is altered by substitution, deletion or insertion, wherein the function of the one or more altered RNA elements is sub-optimal.

In some embodiments, a reference 5′UTR comprises a nucleotide sequence identified by SEQ ID NO: 45 or SEQ ID NO: 4. In some embodiments, a reference 5′UTR comprises a nucleotide sequence that is at least 50%, at least 60%, at least 70%, at least 80%, at least 85%, at least 90%, at least 95%, at least 96%, at least 97%, at least 98%, at least 99%, or 100% identical to the nucleotide sequence identified by SEQ ID NO: 45 or SEQ ID NO: 4. In some embodiments, a reference 5′UTR comprises a nucleotide sequence identical or equivalent to the nucleotide sequence of the test 5′UTR, wherein one or more functional RNA elements of the test 5′UTR are altered by substitution, deletion or insertion, wherein the function of the one or more altered RNA elements is sub-optimal.

Measuring mRNA Stability

In some aspects, the disclosure provides mRNAs comprising a NEMP-derived 3′UTR, a 5′UTR comprising one or more functional RNA elements, or a combination thereof wherein the 5′UTR, the 3′UTR, or both promote, increase, or activate stability of the mRNA when compared to an equivalent mRNA comprising a reference 3′UTR, a reference 5′UTR, or a combination thereof.

In some embodiments, increased stability is determined by measuring the half-life of an mRNA, wherein increased mRNA half-life is indicative of increased stability. Methods of measuring mRNA half-life are known in the art. In some embodiments, mRNA half-life is measured for an mRNA comprising an ORF that encodes a reporter protein, wherein the mRNA half-life is determined by measuring the expression of the reporter protein over time in cells contacted with the mRNA. In some embodiments, a reporter protein is a fluorescent protein, wherein expression of the reporter protein is quantified by measuring the mean fluorescence intensity of contacted cells at regular intervals over time. In some embodiments, a reporter protein is a bioluminescent protein, wherein expression of the reporter protein is quantified by measuring the bioluminescent signal of contacted cells at regular intervals over time. In some embodiments, a reporter protein is an enzyme, wherein expression of the reporter protein is quantified by measuring the level of an enzymatic product present in contacted cells at regular intervals over time. In some embodiments, a reporter protein is recognized by a specific antibody, wherein expression of the reporter protein over time is determined by quantitative immunoblotting using an antibody specific to the reporter protein. Analysis of expression of a reporter protein at regular intervals over time is used to determine the half-life of an mRNA encoding the reporter protein.

In some embodiments, mRNA half-life is measured by a method of RNA quantification, wherein the quantity of a specific mRNA in contacted cells is measured by a method of RNA quantification at regular intervals over time. Methods of RNA quantification are known in the art. Non-limiting examples of RNA quantification include northern analysis, nuclease protection assays, fluorescent in situ hybridization, quantitative real time PCR (RT-PCR), and branched DNA assay.

In some embodiments, the half-life of an mRNA of the disclosure comprising a NEMP-derived 3′UTR, a 5′UTR comprising one or more functional RNA elements, or a combination thereof is increased relative to an equivalent mRNA comprising a reference 3′UTR, a reference 5′UTR, or a combination thereof. In some embodiments, the half-life following administration of a single dose of mRNA is increased by at least 2-fold, 3-fold, 4-fold, 5-fold, 6-fold, 7-fold, 8-fold, 9-fold, 10-fold, 20-fold, 30-fold, 40-fold, or 50-fold for an mRNA comprising a NEMP-derived 3′UTR, a 5′UTR comprising one or more functional RNA elements, or combination thereof relative to a reference mRNA. In some embodiments, the half-life following administration of a single dose of mRNA is increased by at least 10%, at least 20%, at least 30%, at least 40%, at least 50%, at least 60%, at least 70%, at least 75%, 80%, at least 85%, at least 90%, at least 95%, or at least 100% for an mRNA comprising a NEMP-derived 3′UTR, a 5′UTR comprising one or more functional RNA elements, or combination thereof relative to a reference mRNA.

Measuring mRNA Localization

In some aspects, the disclosure provides mRNAs comprising a NEMP-derived 3′UTR, a 5′UTR comprising one or more functional RNA elements, or a combination thereof wherein the 5′UTR, the 3′UTR, or both promote, increase, or activate cellular localization of the mRNA when compared to an equivalent mRNA comprising a reference 3′UTR, a reference 5′UTR, or a combination thereof. In some embodiments, trafficking of an mRNA to certain cellular compartments enables localized translation of the mRNA. In some embodiments, cellular localization is measured in a certain compartment of the cell (e.g., the mitochondria, the endoplasmic reticulum (ER)) relative to the cytosol. Methods of measuring cellular localization in a certain compartment of the cell (e.g., the mitochondria, the ER) is determined by methods and techniques known to one of ordinary skill in the art. For example, biochemical or subcellular fractionation of ribosome populations (e.g., free cytoplasmic ribosomes; mitochondrion-associated ribosomes; ER-associated ribosomes) and subsequent analysis of co-fractionated RNAs (e.g., by Northern blot) have been used to identify mRNAs that associate with mitochondria and/or the ER (see e.g., Margeot et al., (2002) EMBO J 21:6893-6904; Jagannathan et al. (2011) Methods Mol Biol 714:301-321). RNAs (e.g., mRNAs) that co-fractionate or co-purify with certain cellular organelles (e.g., mitochondria, ER) can also be determined by any suitable method to identify, quantify, and/or determine the sequence of the RNAs (e.g., microarrays, RT-PCR, RNAseq, ribosome profiling).

Imaging methods and techniques suitable for the determination of mRNA subcellular localization include, but are not limited to, fluorescent in situ hybridization, fluorescent microscopy, and electron microscopy (EM). RNA fluorescent in situ hybridization (FISH) is advantageous in its ability to detect the localization of endogenous, unmodified mRNA transcripts (see e.g., Burke et al., (2017) ACS Cent Sci 3(5):425-433). Alternatively, mRNA tagging methodologies (e.g., MS2-MCP system) are available to determine association of an mRNA certain cellular organelles (e.g., mitochondria, ER). For example, the 3′UTR of an mRNA of interest is fused to MS2 coat-protein binding sites, and co-expressed with MS2 coat protein that had been fused to a fluorescent protein (MS2-GFP) in a cell. As the fusion MS2-GFP binds to the mRNA of interest, fluorescent microscopy is then used to detect sites of mRNA localization (see e.g., Tutucci et al, (2018) Nat Methods 15(1):81-89). A modification of this method has allowed the detection of endogenously expressed mRNAs, wherein MS2-binding sites are introduced into genomic loci by homologous recombination. In this way, the transcripts are tagged with only minimal interference to their native functions (e.g., expression level) (see. e.g., Haim-Vilmovsky et al., (2011) RNA 17:2249-2255).

Proximity-specific ribosome profiling method allows the isolation and high-throughput characterization of mRNAs that are translated by mitochondria-associated ribosomes and/or ER-associated ribosomes. The basis of proximity-specific ribosome profiling is selective biotinylation of ribosomes in a manner that depends on their subcellular location in intact, unperturbed cells. The use of in vivo labeling allows the recovery of ribosomes from defined locations (e.g., on the surface of mitochondria, on the surface of the ER), including those that cannot be purified by classical cell fractionation techniques. Combining this purification strategy with ribosome profiling provides a tool for the identification of locally translated transcripts and sub-codon monitoring of translation at the site of interest. This has led to the identification of many mRNAs that are translated near the mitochondria and/or the ER, particularly of those that encode inner-membrane proteins (see e.g., Williams et al., (2014) Science 346:748-751 and Jan et al., (2014) Science 346:1257521).

Measuring mRNA Translation Initiation

In some aspects, the disclosure provides mRNAs comprising a NEMP-derived 3′UTR, a 5′UTR comprising one or more functional RNA elements, or a combination thereof wherein the 5′UTR, the 3′UTR, or both promote, increase, or activate a desired translational regulatory activity when compared to an equivalent mRNA comprising a reference 3′UTR, a reference 5′UTR, or a combination thereof. In some embodiments, a desired translational regulatory activity of the disclosure comprises translation initiation fidelity (e.g., as a result of reduced, inhibited, or decreased leaky scanning).

### Ribosome Profiling

In one aspect, RNA elements that provide a desired translational regulatory activity, including modulation of leaking scanning, to polynucleotides e.g., mRNA, are identified and/or characterized by ribosome profiling.

Ribosome profiling is a technique that allows the determination of the number and position of ribosomes bound to mRNAs (see e.g., Ingolia et al., (2009) Science 324(5924):218-23, incorporated herein by reference). The technique is based on protection by the ribosome of a region or segment of mRNA from ribonuclease digestion, which region or segment is subsequently assayed. In this approach, a cell lysate is treated with ribonucleases, leading to generation of 80S ribosomes with fragments of mRNA to which they are bound. The 80S ribosomes are then purified by techniques known in the art (e.g., density gradient centrifugation), and mRNA fragments that are protected by the ribosomes are isolated. Protection results in the generation of a 30-bp fragment of RNA termed a ‘footprint’. The number and sequence of RNA footprints can be analyzed by methods known in the art (e.g., Ribo-seq, RNA-seq). The footprint is roughly centered on the A-site of the ribosome. During translation, a ribosome may dwell at a particular position or location along an mRNA (e.g., at an initiation codon). Footprints generated at these dwell positions are more abundant than footprints generated at positions along the mRNA where the ribosome is more processive. Studies have shown that more footprints are generated at positions where the ribosome exhibits decreased processivity (dwell positions) and fewer footprints where the ribosome exhibits increased processivity (Gardin et al., (2014) eLife 3:e03735). High-throughput sequencing of these footprints provides information on the mRNA locations (sequence of footprints) of ribosomes and generates a quantitative measure of ribosome density (number of footprints comprising a particular sequence) along an mRNA. Accordingly, ribosome profiling data provides information that can be used to identify and/or characterize RNA elements that provide a desired translational regulatory activity of the disclosure, including those that reduce leaky scanning, to polynucleotides as described herein e.g., mRNA.

Ribosome profiling can also be used to determine the extent of ribosome density (aka “ribosome loading”) on an mRNA. It is known that dissociated ribosomal subunits initiate translation at the initiation codon within the 5′-terminal region of mRNA. Upon initiation, the translating ribosome moves along the mRNA chain toward the 3′-end of mRNA, thus vacating the initiation site for loading the next ribosome on the mRNA. In this way a group of ribosomes moving one after another and translating the same mRNA chain is formed. Such a group is referred to as a “polyribosome” or “polysome” (Warner et al., (1963) Proc Natl Acad Sci USA 49:122-129). The number of different mRNA fragments protected by ribosomes per mRNA, per region of an mRNA (e.g., a 5′ UTR), or per location in an mRNA (e.g., an initiation codon) indicates an extent of ribosome density. In general, an increase in the number of ribosomes bound to an mRNA (i.e. ribosome density) is associated with increased levels of protein synthesis.

Accordingly, in some embodiments, an increase in ribosome density of a polynucleotide (e.g., an mRNA) comprising one or more of the modifications or RNA elements of the disclosure, relative to a polynucleotide (e.g., an mRNA) that does not comprise the one or more modifications or RNA elements, is determined by ribosome profiling. In some embodiments, an increase in ribosome density of a polynucleotide (e.g., an mRNA) comprising a structural RNA element (e.g., RNAse P stem loop) of the disclosure, relative to a polynucleotide (e.g., an mRNA) that does not comprise the structural RNA element (e.g., RNAse P stem loop), is determined by ribosome density.

Ribosome profiling is also used to determine the time, extent, rate and/or fidelity of ribosome decoding of a particular codon of an mRNA (and by extension the expected number of corresponding RNA-seq reads in a library of isolated footprints), which in turn is determined by the amount of time a ribosome spends at a particular codon (dwell time). The latter is referred to as a “codon elongation rate” or a “codon decoding rate”. Relative dwell time of ribosomes between two locations in an mRNA, instead of the actual or absolute dwell time at a single location, can also be determined by the comparing the number of sequencing reads of protected mRNA fragments at each location (e.g., a codon) (O'Connor et al., (2016) Nature Commun 7:12915). For example, initiation of polypeptide synthesis at or from an initiation codon can be determined from an observed increase in dwell time of ribosomes at the initiation codon relative to dwell time of ribosomes at a downstream alternate or alternative initiation codon in an mRNA. Accordingly, initiation of polypeptide synthesis at or from an initiation codon in a polynucleotide (e.g., an mRNA) comprising one or more modifications or RNA elements of the disclosure, relative to a polynucleotide (e.g., an mRNA) that does not comprise the one or more modifications or RNA elements, can be determined from an observed increase in the dwell time of ribosomes at the initiation codon relative to the dwell time of ribosomes at a downstream alternate or alternative initiation codon in each polynucleotide (e.g., mRNA).

In some embodiments, an increase in residence time or the time of occupancy (dwell time) of a ribosome at a discrete position or location (e.g., an initiation codon) along a polynucleotide (e.g., an mRNA) comprising one or more of the modifications or RNA elements of the disclosure, relative to a polynucleotide (e.g., an mRNA) that does not comprise the one or more modifications or RNA elements, is determined by ribosome profiling. In some aspects, an increase in residence time or the time of occupancy of a ribosome at an initiation codon in a polynucleotide (e.g., mRNA) comprising a structural RNA element (e.g., RNAse P stem loop) of the disclosure relative to a polynucleotide (e.g., mRNA) that does not comprise the structural RNA element (e.g., RNAse P stem loop), is determined by ribosome profiling.

In other aspects, an increase in the initiation of polypeptide synthesis at or from the initiation codon in polynucleotide (e.g., an mRNA) comprising one or more of the modifications or RNA elements of the disclosure, relative to a polynucleotide (e.g., an mRNA) that does not comprise the one or more modifications or RNA elements, is determined by ribosome profiling. In some embodiments, an increase in the initiation of polypeptide synthesis at or from the initiation codon in a polynucleotide (e.g., mRNA) comprising a structural RNA element (e.g., RNAse P stem loop) of the disclosure relative to a polynucleotide (e.g., mRNA) that does not comprise the structural RNA element (e.g., RNAse P stem loop), is determined by ribosome profiling.

In some embodiments, an increase in fidelity of initiation codon decoding by the ribosome of a polynucleotide (e.g., an mRNA) comprising one or more of the modifications or RNA elements of the disclosure, relative to a polynucleotide (e.g., mRNA) that does not comprise the one or more modifications or RNA elements, is determined by ribosome profiling. In some embodiments, an increase in fidelity of initiation codon decoding by the ribosome of a polynucleotide (e.g., mRNA) comprising a structural RNA element (e.g., RNAse P stem loop) of the disclosure relative to a polynucleotide (e.g., mRNA) that does not comprise the structural RNA element (e.g., RNAse P stem loop), is determined by ribosome profiling.

In some embodiments, an increase in fidelity of initiation codon decoding by the ribosome of a polynucleotide (e.g., an mRNA) comprising one or more of the modifications or RNA elements of the disclosure, relative to a polynucleotide (e.g., an mRNA) that does not comprise the one or more modifications or RNA elements, is determined by ribosome profiling. In some embodiments, an increase in fidelity of initiation codon decoding by the ribosome in a polynucleotide (e.g., mRNA) comprising a structural RNA element (e.g., RNAse P stem loop) of the disclosure relative to a polynucleotide (e.g., mRNA) that does not comprise the structural RNA element (e.g., RNAse P stem loop), is determined by ribosome profiling.

In some embodiments, a decrease in a rate of decoding an initiation codon by the ribosome of a polynucleotide (e.g., an mRNA) comprising one or more of the modifications or RNA elements of the disclosure, relative to a polynucleotide (e.g., an mRNA) that does not comprise the one or more modifications or RNA elements, is determined by ribosome profiling. In some embodiments, a decrease in a rate of decoding an initiation codon by the ribosome of a polynucleotide (e.g., mRNA) comprising a structural RNA element (e.g., RNAse P stem loop) of the disclosure relative to a polynucleotide (e.g., mRNA) that does not comprise the structural RNA element (e.g., RNAse P stem loop), is determined by ribosome profiling.

### Small Ribosomal Subunit Mapping

In some aspects, RNA elements that provide a desired translational regulatory activity, including modulation of leaking scanning, to polynucleotides e.g., mRNA, are identified and/or characterized by small ribosomal subunit mapping.

Small ribosomal subunit (SSU) mapping is a technique similar to ribosome profiling that allows the determination of the number and position of small 40S ribosomal subunits or pre-initiation complexes (PICs) comprising small 40S ribosomal subunits bound to mRNAs. Similar to the technique of ribosome profiling described herein, small ribosomal subunit mapping involves analysis of a region or segment of mRNA protected by the 40S subunit from ribonuclease digestion, resulting in a ‘footprint’, the number and sequence of which can be analyzed by methods known in the art (e.g., RNA-seq). As described herein, the current model of mRNA translation initiation postulates that the pre-initiation complex (alternatively “43S pre-initiation complex”; abbreviated as “PIC”) translocates from the site of recruitment on the mRNA (typically the 5′ cap) to the initiation codon by scanning nucleotides in a 5′ to 3′ direction until the first AUG codon that resides within a specific translation-promotive nucleotide context (the Kozak sequence) is encountered (Kozak (1989) J Cell Biol 108:229-241). “Leaky scanning” by the PIC, whereby the PIC bypasses the initiation codon of an mRNA and instead continues scanning downstream until an alternate or alternative initiation codon is recognized, can occur and result in a decrease in translation efficiency and/or the production of an undesired, aberrant translation product. Thus, analysis of the number of SSUs positioned, or mapped, over AUGs downstream of the first AUG in an mRNA allows for the determination of the extent or frequency at which leaky scanning occurs. SSU mapping provides information that can be used to identify or determine a characteristic (e.g., a translational regulatory activity) of a modification or RNA element of the disclosure, that affects the activity of a small 40S ribosomal subunit (SSU or a PIC comprising the SSU.

Accordingly, an inhibition or reduction of leaky scanning by an SSU or a PIC comprising an SSU of a polynucleotide (e.g., an mRNA) comprising one or more of the modifications or RNA elements of the disclosure, relative to a polynucleotide (e.g., an mRNA) that does not comprise the one or more modifications or RNA elements, is determined by small ribosomal subunit mapping. In some aspects, an inhibition or reduction of leaky scanning by an SSU or a PIC comprising an SSU of a polynucleotide (e.g., an mRNA) comprising a structural RNA element (e.g., RNAse P stem loop) of the disclosure, relative to a polynucleotide (e.g., an mRNA) that does not comprise the structural RNA element (e.g., RNAse P stem loop), is determined by small ribosomal subunit mapping.

In some embodiments, an increase in residence time or the time of occupancy (dwell time) of an SSU or a PIC comprising an SSU at a discrete position or location (e.g., an initiation codon) along a polynucleotide (e.g. an mRNA) comprising one or more of the modifications or RNA elements of the disclosure, relative to a polynucleotide (e.g., an mRNA) that does not comprise the one or more modifications or RNA elements, is determined by ribosome profiling. In some embodiments, an increase in residence time or the time of occupancy of an SSU or a PIC comprising an SSU at an initiation codon in a polynucleotide (e.g., an mRNA) comprising a structural RNA element (e.g., RNAse P stem loop) of the disclosure, relative to a polynucleotide (e.g., an mRNA) that does not comprise the structural RNA element (e.g., RNAse P stem loop), is determined by ribosome profiling.

In some embodiments, an increase in the initiation of polypeptide synthesis at or from the initiation codon in polynucleotide (e.g., an mRNA) comprising one or more of the modifications or RNA elements of the disclosure, relative to a polynucleotide (e.g., an mRNA) that does not comprise the one or more modifications or RNA elements, is determined by ribosome profiling. In some embodiments, an increase in the initiation of polypeptide synthesis at or from the initiation codon in a polynucleotide (e.g., an mRNA) comprising a structural RNA element (e.g., RNAse P stem loop) of the disclosure, relative to a polynucleotide (e.g., an mRNA) that does not comprise the structural RNA element (e.g., RNAse P stem loop), is determined by ribosome profiling.

In some embodiments, an increase in fidelity of initiation codon decoding by an SSU or a PIC comprising an SSU of a polynucleotide (e.g., an mRNA) comprising one or more of the modifications or RNA elements of the disclosure, relative to a polynucleotide that does not comprise the one or more modifications or RNA elements, is determined by ribosome profiling. In some embodiments, an increase in fidelity of initiation codon decoding by an SSU or a PIC comprising an SSU of a polynucleotide (e.g., an mRNA) comprising a structural RNA element (e.g., RNAse P stem loop) of the disclosure, relative to a polynucleotide (e.g., an mRNA) that does not comprise the structural RNA element (e.g., RNAse P stem loop), is determined by ribosome profiling.

In some embodiments, an increase in fidelity of initiation codon decoding by an SSU or a PIC comprising an SSU of a polynucleotide (e.g., an mRNA) comprising one or more of the modifications or RNA elements of the disclosure, relative to a polynucleotide that does not comprise the one or more modifications or RNA elements, is determined by ribosome profiling. In some embodiments, an increase in fidelity of initiation codon decoding by an SSU or a PIC comprising an SSU of a polynucleotide (e.g., an mRNA) comprising a structural RNA element (e.g., RNAse P stem loop) of the disclosure, relative to a polynucleotide (e.g., an mRNA) that does not comprise the structural RNA element (e.g., RNAse P stem loop), is determined by ribosome profiling.

In some embodiments, a decrease in a rate of decoding an initiation codon comprising a polynucleotide (e.g., an mRNA) comprising any one or more of the modifications or RNA elements of the disclosure, relative to a polynucleotide (e.g., an mRNA) that does not comprise the one or more modifications or RNA elements, is determined by ribosome profiling. In some embodiments, a decrease in a rate of decoding an initiation codon decoding by the ribosome of a polynucleotide (e.g., an mRNA) comprising a structural RNA element (e.g., RNAse P stem loop) of the disclosure, relative to a polynucleotide (e.g., an mRNA) that does not comprise the structural RNA element (e.g., RNAse P stem loop), is determined by ribosome profiling.

### RiboFrame-Seq

In some aspects, RNA elements that provide a desired translational regulatory activity, including modulation of leaking scanning, to polynucleotides e.g., mRNA, are identified and/or characterized by RiboFrame-seq.

RiboFrame-seq is an assay that allows for the high-throughput measurement of leaky scanning for many different 5′-UTR sequences. A population of mRNAs is generated with a library of different 5′ UTR sequences, each of which contains a 5′ cap and a coding sequence that encodes a polypeptide comprising two to three different epitope tags, each in a different frame and preceded by an AUG. The mRNA population is transfected into cells and allowed to be translated. Cells are then lysed and immunoprecipitations performed against each of the encoded epitope tags. Each of these immunoprecipitations is designed to isolate a nascent polypeptide chain encoding the particular epitope, as well as the active ribosome performing its synthesis, and the mRNA that encodes it. The complement of 5′-UTRs present in each immunoprecipitate is then analyzed by methods known in the art (e.g., RNA-seq). The 5′-UTRs comprising sequences (e.g. RNA elements) that correlate with reduced, inhibited or low leaky scanning are characterized by being abundant in the immunoprecipitate corresponding to the first epitope tag relative to the other immunoprecipitates.

Accordingly, in some embodiments, a modification or RNA element having a translational regulatory activity of the disclosure is identified or characterized by RiboFrame-seq. In some aspects, a modification or RNA element having reduced, inhibited or low leaky scanning when located in a 5′ UTR of an mRNA are identified or characterized by being abundant in the immunoprecipitate corresponding to the first epitope tag relative to the other immunoprecipitates as determined by RiboFrame-seq.

### Western Blot (Immunodetection)

In some aspects, the disclosure provides a method of identifying, isolating, and/or characterizing a modification (e.g., an RNA element) that provides a translational regulatory activity by synthesizing a 1st control mRNA comprising a polynucleotide sequence comprising an open reading frame encoding a reporter polypeptide (e.g., eGFP) and a 1st AUG codon upstream of, in-frame, and operably linked to, the open reading frame encoding the reporter polypeptide. The 1st control mRNA also comprises a coding sequence for a first epitope tag (e.g. 3xFLAG) upstream of, in-frame, and operably linked to the 1st AUG codon, a 2nd AUG codon upstream of, in-frame, and operably linked to, the coding sequence for the first epitope tag. Optionally, the 1st control mRNA further comprises a coding sequence for a second epitope tag (e.g. V5) upstream of, in-frame, and operably linked to the 2nd AUG codon, and a 3rd AUG codon upstream of, in-frame, and operably linked to, the coding sequence for the second epitope tag. The 1st control mRNA also comprises a 5′ UTR and a 3′ UTR. The method further comprises synthesizing a 2nd test mRNA comprising a polynucleotide sequence comprising the 1st control mRNA and further comprising a modification (e.g. an RNA element). The method further comprises introducing the 1st control mRNA and 2nd test mRNA to conditions suitable for translation of the polynucleotide sequence encoding the reporter polypeptide. The method further comprises measuring the effect of the candidate modification on the amount of reporter polypeptide from each of the three AUG codons. Following transfection of this mRNA into cells, the cell lysate is analyzed by Western blot using antibodies that specifically bind to and detect the reporter polypeptide. This analysis generates two or three bands: a higher band that corresponds to protein generated from the first AUG and lower bands derived from protein generated from the second AUG and, optionally, third AUG.

Leaky scanning is calculated as abundance of the lower bands divided by the sum of the abundance of both bands, as determined by methods known in the art (e.g. densitometry). A test mRNA comprising one or more modifications or RNA elements of the disclosure, that correlate with reduced, inhibited or low leaky scanning is characterized by an increase in amount of polypeptide comprising the second epitope tag compared to the amount of polypeptide that does not comprise an epitope tag, optionally, the amount of polypeptide comprising the first epitope tag, translated from the test mRNA, relative to the control mRNA that does not comprise the one or more modifications or RNA elements. Accordingly, in some embodiments, a modification or RNA element having a translational regulatory activity of the disclosure, is identified by Western blot.

In some embodiments, an inhibition or reduction in leaky scanning of a polynucleotide (e.g., an mRNA) comprising one or more of the modifications or RNA elements of the disclosure, relative to a polynucleotide (e.g., an mRNA) that does not comprise the one or more modifications or RNA elements, is determined by Western blot. In some embodiments, an inhibition or reduction in leaky scanning of a polynucleotide (e.g., an mRNA) comprising a structural RNA element (e.g., RNAse P stem loop) of the disclosure, relative to a polynucleotide (e.g., an mRNA) that does not comprise the structural RNA element (e.g., RNAse P stem loop), is determined by Western blot.

In some embodiments, an increase in the initiation of polypeptide synthesis at or from the initiation codon comprising a polynucleotide (e.g., an mRNA) comprising any one or more of the modifications or RNA elements of the disclosure, relative to a polynucleotide that does not comprise the one or more modifications or RNA elements, is determined by Western blot. In some embodiments, an increase in the initiation of polypeptide synthesis at or from the initiation codon comprising a polynucleotide (e.g., an mRNA) comprising a structural RNA element (e.g., RNAse P stem loop) of the disclosure, relative to a polynucleotide (e.g., an mRNA) that does not comprise the structural RNA element (e.g., RNAse P stem loop), is determined by Western blot.

In some embodiments, an increase in an amount of polypeptide translated from the full open reading frame comprising a polynucleotide (e.g., an mRNA) comprising any one or more of the modifications or RNA elements of the disclosure, relative to a polynucleotide (e.g., an mRNA) that does not comprise the one or more modifications or RNA elements, is determined by Western blot. In some embodiments, an increase in an amount of polypeptide translated from the full open reading frame comprising a polynucleotide (e.g., an mRNA) comprising a structural RNA element (e.g., RNAse P stem loop) of the disclosure, relative to a polynucleotide (e.g., an mRNA) that does not comprise the structural RNA element (e.g., RNAse P stem loop), is determined by Western blot.

In some embodiments, an inhibition or reduction in an amount of polypeptide translated from any open reading frame other than a full open reading frame comprising a polynucleotide (e.g., an mRNA) comprising one or more of the modifications or RNA elements of the disclosure, relative to a polynucleotide (e.g., an mRNA) that does not comprise the one or more modifications or RNA elements, is determined by Western blot. In some embodiments, an inhibition or reduction in an amount of polypeptide translated from any open reading frame other than a full open reading frame comprising a polynucleotide (e.g., an mRNA) comprising a structural RNA element (e.g., RNAse P stem loop) of the disclosure, relative to a polynucleotide (e.g., an mRNA) that does not comprise the structural RNA element (e.g., RNAse P stem loop), is determined by Western blot.

In some embodiments, an inhibition or reduction in the production of aberrant translation products translated from a polynucleotide (e.g., an mRNA) comprising any one or more of the modifications or RNA elements of the disclosure, relative to a polynucleotide (e.g., an mRNA) that does not comprise the one or more modifications or RNA elements, is determined by Western blot. In some embodiments, an inhibition or reduction in the production of aberrant translation products translated from a polynucleotide (e.g., an mRNA) comprising a structural RNA element (e.g., RNAse P stem loop) of the disclosure, relative to a polynucleotide (e.g., an mRNA) that does not comprise the structural RNA element (e.g., RNAse P stem loop), is determined by Western blot.

In some embodiments, leaky scanning by a 43S pre-initiation complex (PIC) or ribosome of a polynucleotide (e.g., an mRNA) comprising one or more of the modifications or RNA elements (e.g., RNAse P stem loop) of the disclosure is decreased by about 80%-100%, about 60%-80%, about 40%-60%, about 20%-40%, about 10%-20%, about 5%-10%, about 1%-5% relative to a polynucleotide (e.g., an mRNA) that does not comprise the one or more modifications or RNA elements, as determined by SSU mapping and/or ribosome profiling methods, as described herein.

In some embodiments, leaky scanning by a 43S pre-initiation complex (PIC) or ribosome of a polynucleotide (e.g., an mRNA) comprising any one or more of the modifications or RNA elements (e.g., RNAse P stem loop) of the disclosure is decreased by about 80%-100%, about 60%-80%, about 40%-60%, about 20%-40%, about 10%-20%, about 5%-10%, about 1%-5% and an amount of a polypeptide translated from a full reading frame is increased by about 80%-100%, about 60%-80%, about 40%-60%, about 20%-40%, about 10%-20%, about 5%-10%, about 1%-5% relative to a polynucleotide (e.g., an mRNA) that does not comprise the one or more modification or RNA elements, as determined by SSU mapping and Western blot, respectively, as described herein.

In some embodiments, leaky scanning by the 43S pre-initiation complex (PIC) or ribosome of a polynucleotide (e.g., an mRNA) comprising any one or more of the modifications or RNA elements (e.g., RNAse P stem loop) of the disclosure is decreased by about 80%-100%, about 60%-80%, about 40%-60%, about 20%-40%, about 10%-20%, about 5%-10%, about 1%-5%, an amount of a polypeptide translated from a full open reading frame is increased by about 80%-100%, about 60%-80%, about 40%-60%, about 20%-40%, about 10%-20%, about 5%-10%, about 1%-5%, and potency of the polypeptide is increased by about 80%-100%, about 60%-80%, about 40%-60%, about 20%-40%, about 10%-20%, about 5%-10%, about 1%-5%, relative to a polynucleotide (e.g., an mRNA) that does not comprise the one or more modification or RNA elements, as determined by SSU mapping and Western blot.

In some aspects, the disclosure provides a reporter system to characterize RNA elements that provide a desired translational regulatory activity. Specifically, a method of identifying RNA elements having translational regulatory activity comprises:
(i) providing a population of polynucleotides, wherein each polynucleotide comprises a plurality of open reading frames encoding a plurality of polypeptides, each comprises a peptide epitope tag, wherein each polynucleotide comprises:
   (a) at least one first AUG codon upstream of, in-frame, and operably linked to at least one first open reading frame encoding at least one first polypeptide comprising at least one first peptide epitope tag;
   (b) at least one second AUG codon upstream of, in-frame, and operably linked to at least one second open reading frame encoding at least one second polypeptide comprising at least one second peptide epitope tag, wherein the second AUG codon is downstream and out-of-frame of the first AUG codon; optionally,
   (c) at least one third AUG codon upstream of, in-frame, and operably linked to at least one third open reading frame encoding at least one third polypeptide comprising at least one second peptide epitope tag, wherein the third AUG codon is downstream and out-of-frame with the first and second AUG codons; and
   (d) a 5′UTR and a 3′UTR, wherein the 5′UTR of each polynucleotide within the population comprises a unique nucleotide sequence;
   (e) no stop codons (UAG, UGA, or UAA) within any frame between the first AUG and the stop codon corresponding to the first AUG;
(ii) providing conditions suitable for translation of each polynucleotide in the population of polynucleotides;
(iii) isolating a complex comprising a nascent translation product comprising the first, second and, if present, third epitope tag, and the 5′ UTR corresponding to the epitope tag and encoded polynucleotide;
(iv) determining the sequences of the 5′UTRs corresponding to each polynucleotide encoding the nascent translation product; and
(v) determining which nucleotides are enriched at each position in the 5′UTR of the first polynucleotide compared to the second, and optionally third, polynucleotide.

In some embodiments, the first polynucleotide encodes a reporter polypeptide, such as eGFP. In some embodiments, the first AUG is linked to and in frame with an open reading frame that encodes eGFP. Reporter polypeptides are known to those of skill in the art.

In some embodiments, the peptide epitope tag is selected from the group consisting of: a FLAG tag (DYKDDDDK; SEQ ID NO: 155); a 3xFLAG tag (DYKDHDGDYKDHDIDYKDDDK; SEQ ID NO: 156); a Myc tag (EQKLISEEDL; SEQ ID NO: 184); a V5 tag (GKPIPNPLLGLDST; SEQ ID NO: 185); a hemagglutinin A (HA) tag (YPYDVPDYA; SEQ ID NO: 186); a histidine tag (e.g., a 6xHis tag; HHHHHH; SEQ ID NO: 187); an HSV tag (QPELAPEDPED; SEQ ID NO: 188); a VSV-G tag (YTDIEMNRLGK; SEQ ID NO: 189); an NE tag (TKENPRSNQEESYDDNES; SEQ ID NO: 190); an AViTag (GLNDIFEAQKIEWHE; SEQ ID NO: 191); a calmodulin tag (KRRWKKNFIAVSAANRFKKISSSGAL; SEQ ID NO: 192); an E tag (GAPVPYPDPLEPR; SEQ ID NO: 193); an S tag (KETAAAKFERQHMDS; SEQ ID NO: 194); an SBP tag (MDEKTTGWRGGHVVEGLAGELEQLRARLEHHPQGQREP; SEQ ID NO: 195); a Softag 1 (SLAELLNAGLGGS; SEQ ID NO: 196); a Softag 3 (TQDPSRVG; SEQ ID NO: 197); a Strep tag (WSHPQFEK; SEQ ID NO: 198); a Ty tag (EVHTNQDPLD; SEQ ID NO: 199); and an Xpress tag (DLYDDDDK; SEQ ID NO: 200).

Another RNA element known to regulate translation of mRNA is the five-prime cap (5′ cap), which is a specially altered nucleotide the 5′ end of natural mRNA co-transcriptionally. This process, known as mRNA capping, is highly regulated and is vital in the creation of stable and mature messenger RNA able to undergo translation. In eukaryotes, the structure of the 5′ cap consists of a guanine nucleotide connected to 5′ end of an mRNA via an unusual 5′ to 5′ triphosphate linkage. This guanosine is methylated on the 7 position directly after capping in vivo by a methyltransferase, and as such, is sometimes referred to as a 7-methylguanylate cap, and abbreviated m7G. A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G(5′)ppp(5′)G, commonly written as m7GpppG. A cap species may also be an anti-reverse cap analog. A non-limiting list of possible cap species includes m7GpppG, m7Gpppm7G, m73′dGpppG, m27,O3′GpppG, m27,O3′GppppG, m27,O2′GppppG, m7Gpppm7G, m73′dGpppG, m27,O3′GpppG, m27,O3′GppppG, and m27,O2′GppppG. Accordingly, in some embodiments, the mRNAs disclosed herein comprise a 5′ cap, or derivative, analog, or modification thereof.

An early event in translation initiation involves the formation of the 43S pre-initiation complex (PIC) composed of the small 40S ribosomal subunit, the initiator transfer RNA (Met-tRNAiMet), and several various eIFs. Following recruitment to the mRNA, the PIC biochemically interrogates or “scans” the sequence of the mRNA molecule in search of an initiation codon. In some embodiments of the mRNAs disclosed herein, the mRNAs comprise at least one initiation codon. In some embodiments, the initiation codon is an AUG codon. In some embodiments, the initiation codon comprises one or more modified nucleotides.

Similar to polypeptides, polynucleotides, particularly RNA, can fold into a variety of complex three dimensional structures. The ability of a nucleic acid to form a complex, functional three dimensional structure is exemplified by a transfer RNA molecule (tRNA), which is a single chain of ˜70-90 nucleotides in length that folds into an L-shaped 3D structure allowing it to fit into the P and A sites of a ribosome and function as the physical link between the polypeptide coding sequence of mRNA and the amino acid sequence of the polypeptide. Since base pairing between complementary sequences of nucleobases determines the overall secondary (and ultimately tertiary) structure of nucleic acid molecules, sequences predicted to or known to be able to adopt a particular structure (e.g. a stem-loop) are vital considerations in the design and utility of some types of functional elements or motifs (e.g. RNA elements). Nucleic acid secondary structure is generally divided into duplexes (contiguous base pairs) and various kinds of loops (unpaired nucleotides flanked or surrounded by duplexes). As is known in the art, stable RNA secondary structures, or combinations of them, can be further classified and usefully described as, but not limited to, simple loops, tetraloops, pseudoknots, hairpins, helicies, and stem-loops. Secondary structure can also be usefully depicted as a list of nucleobases which are paired in a nucleic acid molecule.

The function(s) of a nucleic acid secondary structure are emergent from the thermodynamic properties of the secondary structure. For example, the thermodynamic stability of an RNA hairpin/stemloop structure is characterized by its free energy change (deltaG). For a spontaneous process, i.e. the formation of a stable RNA hairpin/stemloop, deltaG is negative. The lower the deltaG value, the more energy is required to reverse the process, i.e. the more energy is required to denature or melt (‘unfold’) the RNA hairpin/stemloop. The stability of an RNA hairpin/stemloop will contribute to its biological function: e.g. in the context of translation, a more stable RNA structure with a relatively low deltaG can act a physical barrier for the ribosome (Kozak, 1986; Babendure et al., 2006), leading to inhibition of protein synthesis. In contrast, a weaker or moderately stable RNA structure can be beneficial as translational enhancer, as the translational machinery will recognize it as signal for a temporary pause, but ultimately the structure will open up and allow translation to proceed (Kozal, 1986; Kozak, 1990; Babendure et al., 2006). To assign an absolute number to the deltaG value that defines a stable versus a weak/moderately stable RNA hairpin/stemloop is difficult and is very much driven by its context (sequence and structural context, biological context). In the context of the above-mentioned examples by Kozak, 1986, Kozak, 1990 and Babendure et al., 2006, stable hairpins/stemloops are characterized by approximate deltaG values lower than −30 kcal/mol, while weak/moderately stable hairpins are characterized by approximate deltaG values between −10 and −30 kcal/mol.

Accordingly, in some embodiments, an mRNA comprises at least one modification, wherein the at least one modification is a structural modification. In some embodiments, the structural modification is an RNA element. In some embodiments, the structural modification is a GC-rich RNA element. In some embodiments, the structural modification is a viral RNA element. In some embodiments, the structural modification is a protein-binding RNA element. In some embodiments, the structural modification is a translation initiation element. In some embodiments, the structural modification is a translation enhancer element. In some embodiments, the structural modification is a translation fidelity enhancing element. In some embodiments, the structural modification is an mRNA nuclear export element. In some embodiments, the structural modification is a stable RNA secondary structure (e.g., an RNAse P stem loop).

The mRNAs of the present disclosure, or regions thereof, may be codon optimized. Codon optimization methods are known in the art and may be useful for a variety of purposes: matching codon frequencies in host organisms to ensure proper folding, bias GC content to increase mRNA stability or reduce secondary structures, minimize tandem repeat codons or base runs that may impair gene construction or expression, customize transcriptional and translational control regions, insert or remove proteins trafficking sequences, remove/add post translation modification sites in encoded proteins (e.g., glycosylation sites), add, remove or shuffle protein domains, insert or delete restriction sites, modify ribosome binding sites and mRNA degradation sites, adjust translation rates to allow the various domains of the protein to fold properly, or to reduce or eliminate problem secondary structures within the polynucleotide. Codon optimization tools, algorithms and services are known in the art, including those described by PCT/US2015/059112 and PCT/US2015/059079 that are incorporated by reference herein; additional non-limiting examples include services from GeneArt (Life Technologies), DNA2.0 (Menlo Park, Calif.) and/or proprietary methods. In one embodiment, the mRNA sequence is optimized using optimization algorithms, e.g., to optimize expression in mammalian cells or enhance mRNA stability. Accordingly, in some embodiments, an mRNA comprises a structural modification, wherein the structural modification is a codon optimized open reading frame. In some embodiments, the structural modification is a modification of base composition.

Methods of Measuring mRNA Expression and Activity of Translated Protein
Measuring mRNA Expression of Translated Protein

In some aspects, the disclosure provides mRNAs comprising a NEMP-derived 3′UTR, a 5′UTR comprising one or more functional RNA elements, or a combination thereof, wherein the expression level and/or an activity of a polypeptide translated from the mRNA is increased relative to an equivalent mRNA comprising a reference 3′UTR, a reference 5′UTR, or a combination thereof. Methods for determining polypeptide expression and/or activity are known to those of skill in the art and are described herein. Such methods include, but are not limited to, quantitative immunofluorescence (QIF), flow cytometry, reverse transcription polymerase chain reaction (RT-PCR), competitive RT-PCR, real-time RT-PCR, RNase protection assay (RPA), northern blotting, nucleic acid microarray using DNA, western blotting, enzyme-linked immunosorbent assay (ELISA), radioimmunoassay (RIA), tissue immunostaining, immunoprecipitation assay, complement fixation assay, fluorescence-activated cell sorting (FACS), mass spectrometry, magnetic bead-antibody immunoprecipitation, protein chip, or biochemical or biomarker assays to determine enzymatic activity in vitro or in vivo.

In some aspects, the disclosure provides mRNAs comprising a NEMP-derived 3′UTR, a 5′UTR comprising one or more functional RNA elements, or a combination thereof, wherein the expression level and/or an activity of a polypeptide translated from the mRNA is increased relative to an equivalent mRNA comprising a reference 3′UTR, a reference 5′UTR, or a combination thereof upon contacting cells in vitro or in vivo. In some embodiments, increased expression level is determined by measuring the level of protein translated from an mRNA. In some embodiments, the level of translated protein produced from an mRNA comprising a NEMP-derived 3′UTR, a 5′UTR comprising one or more functional RNA elements, or combination thereof is measured for an mRNA encoding an ORF that is a reporter protein. The level of protein translated from an mRNA comprising an ORF that encodes a reporter protein is determined by measuring the expression of the reporter protein in cells contacted with the mRNA at regular intervals over time. Analysis of the area under the curve (AUC) of signal of the expressed reporter protein over time provides a measure of the total reporter protein translated from an mRNA in contacted cells. In some embodiments, a reporter protein is a fluorescent protein, wherein the AUC of cellular mean fluorescence intensity over time provides a measure of total reporter protein translated from an mRNA. In some embodiments, a reporter protein is a bioluminescent protein, wherein the AUC of cellular bioluminescent signal over time provides a measure of total reporter protein translated from an mRNA. In some embodiments, a reporter protein is an enzyme, wherein the AUC of cellular enzymatic product produced over time provides a measure of total reporter protein translated from an mRNA. In some embodiments, a reporter protein is recognized by a specific antibody, wherein expression of the reporter protein is measured by quantitate immunoblotting using an antibody specific to the reporter protein. The AUC of cellular reporter protein produced over time as measured by quantitate immunoblot provides a measure of total reporter protein translated from an mRNA.

In some embodiments, the level of translated protein produced from an mRNA comprising a NEMP-derived 3′UTR, a 5′UTR comprising one or more functional RNA elements, or combination thereof is measured using a method of protein quantification. Methods of quantifying translated protein in a cell are known in the art. Non-limiting examples of methods to quantifying cellular proteins translated from an mRNA provided to the cell include high-performance liquid chromatography (HPLC), liquid chromatography-mass spectrometry (LC/MS), matrix assisted laser desorption/ionization time of flight (MALDI-TOF) mass spectrometry, enzyme-linked immunosorbent assay (ELISA), protein immunoprecipitation, and quantitative immunoblotting. In some embodiments, analysis of the AUC of quantity of translated protein in contacted cells over time is used to determine the total level of protein translated from an mRNA.

In some embodiments, the AUC of translated protein over time in contacted cells is increased for mRNA comprising a NEMP-derived 3′UTR, a 5′UTR comprising one or more functional RNA elements, or combination thereof relative to an equivalent mRNA comprising a reference 3′UTR, a reference 5′UTR, or a combination thereof. In some embodiments, the AUC of translated protein over time in contacted cells is increased by at least 2-fold, 3-fold, 4-fold, 5-fold, 6-fold, 7-fold, 8-fold, 9-fold, 10-fold, 20-fold, 30-fold, 40-fold, or 50-fold for mRNA comprising a NEMP-derived 3′UTR, a 5′UTR comprising one or more functional RNA elements, or combination thereof relative to an equivalent mRNA comprising a reference 3′UTR, a reference 5′UTR, or a combination thereof. In some embodiments, the AUC of translated protein over time in contacted cells is increased by at least 10%, at least 20%, at least 30%, at least 40%, at least 50%, at least 60%, at least 70%, at least 75%, 80%, at least 85%, at least 90%, at least 95%, or at least 100% for mRNA comprising a NEMP-derived 3′UTR, a 5′UTR comprising one or more functional RNA elements, or combination thereof relative to an equivalent mRNA comprising a reference 3′UTR, a reference 5′UTR, or a combination thereof. In some embodiments, the AUC of translated protein over time in contacted cells is increased for at least 6 hours, at least 12 hours, at least 24 hours, at least 36 hours, at least 48 hours, at least 60 hours, at least 72 hours, at least 84 hours, at least 96 hours, at least 108 hours, at least 122 hours, at least 144 hours, at least 168 hours, at least 192 hours, at least 240 hours, at least 288 hours, at least 336 hours, at least 384 hours, at least 432 hours, at least 480 hours, at least 504 hours at least 528 hours, at least 672 hours after contacting the cells with a single dose of an mRNA of the disclosure.

Certain aspects of the disclosure feature measurement, determination and/or monitoring of the expression level or levels of an encoded polypeptide of interest in a subject, for example, in an animal (e.g., rodents, primates, and the like) or in a human subject. Animals include normal, healthy or wild-type animals, as well as animal models for use in understanding the pathophysiology or disease state resulting from the deficiency of a polypeptide of interest (e.g., a therapeutic protein, such as a membrane bound, intracellular, or secreted protein).

Expression levels of an encoded polypeptide of interest can be measured or determined by any art-recognized method for determining protein levels in biological samples, e.g., from blood samples or a needle biopsy. The term “level” or “level of a protein” or “level of a polypeptide of interest” as used herein, preferably means the weight, mass or concentration of the protein (e.g., polypeptide of interest) within a sample or a subject. It will be understood by the skilled artisan that in certain embodiments the sample may be subjected, e.g., to any of the following: purification, precipitation, separation, e.g. centrifugation and/or HPLC, and subsequently subjected to determining the level of the protein, e.g., using mass and/or spectrometric analysis. In exemplary embodiments, enzyme-linked immunosorbent assay (ELISA) can be used to determine protein expression levels. In other exemplary embodiments, protein purification, separation and LC-MS can be used as a means for determining the level of a protein according to the invention. In some embodiments, the level of expression of a polypeptide of interest is determined in any tissue collected from a subject, non-limiting examples including bone, blood, heart, kidney, liver, skin, intestine, brain, spleen, thyroid, or lung.

In some embodiments, an mRNA therapy of the disclosure (e.g., a single intravenous dose) results in increased expression level of a polypeptide of interest in a given tissue of the subject (e.g., liver, kidney, or heart) that is 2-fold, 3-fold, 4-fold, 5-fold, 6-fold, 7-fold, 8-fold, 9-fold, 10-fold, 20-fold, 30-fold, 40-fold, 50-fold increase and/or increased to at least 50%, at least 60%, at least 70%, at least 75%, 80%, at least 85%, at least 90%, at least 95%, or at least 100% of normal levels for at least 6 hours, at least 12 hours, at least 24 hours, at least 36 hours, at least 48 hours, at least 60 hours, at least 72 hours, at least 84 hours, at least 96 hours, at least 108 hours, at least 122 hours, at least 144 hours, at least 168 hours, at least 192 hours, at least 240 hours, at least 288 hours, at least 336 hours, at least 384 hours, at least 432 hours, at least 480 hours, at least 504 hours at least 528 hours, at least 672 hours after administration of a single dose of the mRNA therapy. In some embodiments, an mRNA therapy of the disclosure (e.g., a single intravenous dose) results in increased expression level of a polypeptide of interest in a given tissue of the subject (e.g., liver, kidney, or heart) that is 2-fold, 3-fold, 4-fold, 5-fold, 6-fold, 7-fold, 8-fold, 9-fold, 10-fold, 20-fold, 30-fold, 40-fold, 50-fold increase and/or increased to at least 50%, at least 60%, at least 70%, at least 75%, 80%, at least 85%, at least 90%, at least 95%, or at least 100% of normal levels for at least 6 hours, at least 12 hours, at least 24 hours, or at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more days after administration of a single dose of the mRNA therapy.

### Measuring Activity of a Translated Protein

In some patients with a disease or disorder, the enzymatic activity of a polypeptide of interest (e.g., a cellular enzyme) is reduced compared to a normal physiological activity level. Further aspects of the disclosure feature measurement, determination and/or monitoring of the activity level(s) (i.e., enzymatic activity level(s)) of a polypeptide of interest (e.g., a cellular enzyme) in a subject, for example, in an animal (e.g., rodent, primate, and the like) or in a human subject. Activity levels can be measured or determined by any art-recognized method for determining enzymatic activity levels in biological samples. The term “activity level” or “enzymatic activity level” as used herein, preferably means the activity of the enzyme per volume, mass or weight of sample or total protein within a sample. In exemplary embodiments, the “activity level” or “enzymatic activity level” is described in terms of units per milliliter of fluid (e.g., bodily fluid, e.g., serum, plasma, urine and the like) or is described in terms of units per weight of tissue or per weight of protein (e.g., total protein) within a sample. Units (“U”) of enzyme activity can be described in terms of weight or mass of substrate hydrolyzed per unit time. In certain embodiments of the invention feature enzymatic activity described in terms of U/ml plasma or U/mg protein (tissue), where units (“U”) are described in terms of nmol substrate hydrolyzed per hour (or nmol/hr).

In certain embodiments, an mRNA therapy of the disclosure features a pharmaceutical composition comprising a dose of mRNA effective to result in at least 5 U/mg, at least 10 U/mg, at least 20 U/mg, at least 30 U/mg, at least 40 U/mg, at least 50 U/mg, at least 60 U/mg, at least 70 U/mg, at least 80 U/mg, at least 90 U/mg, at least 100 U/mg, or at least 150 U/mg of a enzymatic activity in tissue (e.g., liver) between 6 and 12 hours, or between 12 and 24, between 24 and 48, or between 48 and 72 hours post administration (e.g., at 48 or at 72 hours post administration).

In some embodiments, an mRNA therapy of the disclosure (e.g., a single intravenous dose) results in increased enzymatic activity levels in the liver, kidney or heart tissue of the subject (e.g., 2-fold, 3-fold, 4-fold, 5-fold, 6-fold, 7-fold, 8-fold, 9-fold, 10-fold, 20-fold, 30-fold, 40-fold, 50-fold increase and/or increased to at least 50%, at least 60%, at least 70%, at least 75%, 80%, at least 85%, at least 90%, at least 95%, or at least 100% of normal levels) for at least 6 hours, at least 12 hours, at least 24 hours, or at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more days after administration of a single dose of the mRNA therapy.

In exemplary embodiments, an mRNA therapy of the disclosure features a pharmaceutical composition comprising a single intravenous dose of mRNA that results in the above-described levels of activity. In another embodiment, an mRNA therapy of the disclosure features a pharmaceutical composition which can be administered in multiple single unit intravenous doses of mRNA that maintain the above-described levels of activity.

### Measuring Biomarkers of a Translated Polypeptide of Interest

Further aspects of the disclosure feature determining the level (or levels) of a biomarker, (e.g., a metabolite or enzymatic product produced by a cellular enzyme) determined in a sample as compared to a level (e.g., a reference level) of the same or another biomarker in another sample, e.g., from the same patient, from another patient, from a control and/or from the same or different time points, and/or a physiologic level, and/or an elevated level, and/or a supraphysiologic level, and/or a level of a control. The skilled artisan will be familiar with physiologic levels of biomarkers, for example, levels in normal or wild-type animals, normal or healthy subjects, and the like, in particular, the level or levels characteristic of subjects who are healthy and/or normal functioning. As used herein, the phrase “elevated level” means amounts greater than normally found in a normal or wild-type preclinical animal or in a normal or healthy subject, e.g. a human subject. As used herein, the term “supraphysiologic” means amounts greater than normally found in a normal or wild-type preclinical animal or in a normal or healthy subject, e.g. a human subject, optionally producing a significantly enhanced physiologic response. As used herein, the term “comparing” or “compared to” preferably means the mathematical comparison of the two or more values, e.g., of the levels of the biomarker(s). It will thus be readily apparent to the skilled artisan whether one of the values is higher, lower or identical to another value or group of values if at least two of such values are compared with each other. Comparing or comparison to can be in the context, for example, of comparing to a control value, e.g., as compared to a reference blood plasma, serum, red blood cells (RBC) and/or tissue (e.g., liver, kidney, heart) biomarker level, and/or a reference serum, blood plasma, tissue (e.g., liver, kidney, or heart), and/or urinary biomarker level, in said subject prior to administration (e.g., in a person suffering from a disease or disorder resulting from an enzyme deficiency) or in a normal or healthy subject. Comparing or comparison to can also be in the context, for example, of comparing to a control value, e.g., as compared to a reference blood plasma, serum, red blood cells (RBC) and/or tissue (e.g., liver, kidney, or heart) biomarker level, and/or a reference serum, blood plasma, tissue (e.g., liver), and/or urinary biomarker level in said subject prior to administration (e.g., in a person suffering from a disease or disorder resulting from an enzyme deficiency) or in a normal or healthy subject.

As used herein, a “control” is preferably a sample from a subject wherein the health or disease status of said subject is known. In one embodiment, a control is a sample of a healthy patient. In another embodiment, the control is a sample from at least one subject having a known disease status, for example, a severe, mild, or healthy disease status, e.g. a control patient. In another embodiment, the control is a sample from a subject not being treated for the disease. In a still further embodiment, the control is a sample from a single subject or a pool of samples from different subjects and/or samples taken from the subject(s) at different time points.

The term “level” or “level of a biomarker” as used herein, preferably means the mass, weight or concentration of a biomarker of the disclosure within a sample or a subject. Biomarkers of the disclosure include, for example, metabolites or enzymatic products of a cellular enzyme. It will be understood by the skilled artisan that in certain embodiments the sample may be subjected to, e.g., one or more of the following: substance purification, precipitation, separation, e.g. centrifugation and/or HPLC and subsequently subjected to determining the level of the biomarker, e.g. using mass spectrometric analysis. In certain embodiments, LC-MS can be used as a means for determining the level of a biomarker according to the disclosure.

The term “determining the level” of a biomarker as used herein can mean methods which include quantifying an amount of at least one substance in a sample from a subject, for example, in a bodily fluid from the subject (e.g., serum, plasma, urine, RBC, lymph, fecal, etc.) or in a tissue of the subject (e.g., liver, heart, spleen, kidney, etc.).

The term “reference level” as used herein can refer to levels (e.g., of a biomarker) in a subject prior to administration of an mRNA therapy of the disclosure (e.g., in a person suffering from a disease or disorder resulting from an enzyme deficiency) or in a normal or healthy subject.

As used herein, the term “normal subject” or “healthy subject” refers to a subject not suffering from symptoms associated with a disease or disorder (e.g., a disease or disorder resulting from an enzyme deficiency). Moreover, a subject will be considered to be normal (or healthy) if it has no mutation of the functional portions or domains of a polypeptide of interest (e.g., a cellular enzyme) and/or no mutation of the polypeptide of interest (e.g., cellular enzyme) gene resulting in a reduction of or deficiency of the polypeptide of interest (e.g., cellular enzyme) expression level or the activity thereof. Said mutations will be detected if a sample from the subject is subjected to a genetic testing for such mutations. In certain embodiments of the present disclosure, a sample from a healthy subject is used as a control sample, or the known or standardized value for the level of biomarker from samples of healthy or normal subjects is used as a control.

In some embodiments, comparing the level of the biomarker in a sample from a subject in need of treatment for a disease or disorder (e.g., a disease or disorder resulting from an enzyme deficiency) or in a subject being treated for a disease or disorder (e.g., a disease or disorder resulting from an enzyme deficiency) to a control level of the biomarker comprises comparing the level of the biomarker in the sample from the subject (e.g., in need of treatment or being treated for the disease or disorder) to a baseline or reference level, wherein if a level of the biomarker in the sample from the subject (e.g., in need of treatment or being treated for a disease or disorder) is elevated, increased or higher compared to the baseline or reference level, this is indicative that the subject is suffering from the disease or disorder and/or is in need of treatment; and/or wherein if a level of the biomarker in the sample from the subject (e.g., in need of treatment or being treated for the disease or disorder) is decreased or lower compared to the baseline level this is indicative that the subject is not suffering from, is successfully being treated for the disease or disorder (e.g., a disease or disorder resulting from an enzyme deficiency), or is not in need of treatment for the disease or disorder (e.g., a disease or disorder resulting from an enzyme deficiency). The stronger the reduction (e.g., at least 1.5-fold, at least 2-fold, at least 3-fold, at least 4-fold, at least 5-fold, at least 6-fold, at least 7-fold, at least 8-fold, at least 10-fold, at least 20-fold, at least-30 fold, at least 40-fold, at least 50-fold reduction and/or at least 10%, at least 20%, at least 30% at least 40%, at least 50%, at least 60%, at least 70%, at least 80%, at least 90%, or at least 100% reduction) of the level of a biomarker, within a certain time period, e.g., within 6 hours, within 12 hours, 24 hours, 36 hours, 48 hours, 60 hours, or 72 hours, and/or for a certain duration of time, e.g., 48 hours, 72 hours, 96 hours, 120 hours, 144 hours, 1 week, 2 weeks, 3 weeks, 4 weeks, 1 month, 2 months, 3 months, 4 months, 5 months, 6 months, 7 months, 8 months, 9 months, 10 months, 11 months, 12 months, 18 months, 24 months, etc. the more successful is a therapy, such as for example an mRNA therapy of the disclosure (e.g., a single dose or a multiple regimen).

A reduction of at least about 20%, at least about 30%, at least about 40%, at least about 50%, at least about 60%, at least about 70%, at least about 80%, at least about 90%, at least 100% or more of the level of biomarker, in particular, in bodily fluid (e.g., plasma, serum, red blood cells (RBC), urine, e.g., urinary sediment) or in tissue(s) in a subject (e.g., liver) within 1, 2, 3, 4, 5, 6 or more days following administration is indicative of a dose suitable for successful treatment the disease or disorder (e.g., a disease or disorder resulting from an enzyme deficiency), wherein reduction as used herein, preferably means that the level of biomarker determined at the end of a specified time period (e.g., post-administration, for example, of a single intravenous dose) is compared to the level of the same biomarker determined at the beginning of said time period (e.g., pre-administration of said dose). Exemplary time periods include 12, 24, 48, 72, 96, 120 or 144 hours post administration, in particular 24, 48, 72 or 96 hours post administration.

In some aspects, a sustained reduction in substrate levels of a polypeptide of interest that is a cellular enzyme (e.g., biomarkers such as metabolites or enzymatic products of a cellular enzyme) is particularly indicative of mRNA therapeutic dosing and/or administration regimens successful for treatment of a disease or disorder resulting from an enzyme deficiency. Such sustained reduction can be referred to herein as “duration” of effect. In exemplary embodiments, a reduction of at least about 20%, at least about 30%, at least about 40%, at least about 50%, at least about 60%, at least about 65%, at least about 70%, at least about 75%, at least about 80%, at least about 85%, at least about 90%, or at least about 95% at least about 96%, at least about 97%, at least about 98%, at least about 99%, at least about 100% or more of the level of biomarker, in particular, in a bodily fluid (e.g., plasma, serum, RBC, urine, e.g., urinary sediment) or in tissue(s) in a subject (e.g., liver) within 1, 2, 3, 4, 5, 6, 7, 8 or more days following administration is indicative of a successful therapeutic approach. In exemplary embodiments, sustained reduction in substrate (e.g., biomarker) levels in one or more samples (e.g., fluids and/or tissues) is preferred. For example, mRNA therapies resulting in sustained reduction in a biomarker, optionally in combination with sustained reduction of said biomarker in at least one tissue, preferably two, three, four, five or more tissues, is indicative of successful treatment.

In some embodiments, a single dose of an mRNA therapy of the disclosure is about 0.2 to about 0.8 mpk. about 0.3 to about 0.7 mpk, about 0.4 to about 0.8 mpk, or about 0.5 mpk. In another embodiment, a single dose of an mRNA therapy of the disclosure is less than 1.5 mpk, less than 1.25 mpk, less than 1 mpk, or less than 0.75 mpk.

### Polynucleotides Comprising a Mitochondrial-Targeting Sequence (MTS)

In some aspects, the mRNAs provided by the disclosure comprise an open reading frame (ORF) encoding a polypeptide of interest and a mitochondrial-targeting sequence (MTS). The targeting of a protein to a cellular or subcellular location (e.g., within an organelle such as a mitochondria) is often mediated by an N-terminal ‘topogenic sequence’, which functions in the translocation of the protein to the cellular or subcellular location and across at least one membrane. The majority (>98%) of mitochondrial proteins are encoded in the nucleus of a cell, synthesized as precursor mitochondrial proteins (referred to as “preproteins”) in the cytosol by cytoplasmic ribosomes, and then localized or ‘sorted’ to the mitochondria post-translationally.

A mitochondrial preprotein destined for a mitochondrion is typically synthesized with a cleavable peptide extension at the N-terminus, referred to as a “presequence” or a “mitochondrial-targeting sequence” (MTS). MTSs vary in amino acid composition and length, but are typically of 20-50 amino acid residues in length (with a range of approximately 10-80 residues). The MTSs of different mitochondrial proteins do not show amino-acid sequence identity, but they do have characteristic physicochemical properties. They are enriched in positively charged, hydroxylated and hydrophobic residues, and have the potential to form an amphiphilic a helix. In a helical wheel projection, the positively charged residues localize to one side of the helix, while the opposite side is uncharged and hydrophobic (Roise & Schatz (1988) J Biol Chem 263:4509-4511; von Heijne et al., (1989) Eur J Biochem 180:535-545).

Accordingly, in some embodiments, an MTS of the disclosure is about 10-100 amino acid residues in length. In some embodiments, an MTS is about 10-15 amino acid residues in length, about 15-20 amino acid residues in length, about 20-30 amino acid residues in length, about 30-40 amino acid residues in length, about 40-50 amino acid residues in length, about 50-60 amino acid residues in length, about 60-70 amino acid residues in length, about 70-80 amino acid residues in length, about 80-90 amino acid residues in length, or about 90-100 amino acid residues in length.

MTSs have been shown to target both mitochondrial and non-mitochondrial and/or heterologous proteins to mitochondria and across both outer and inner membranes into the matrix, demonstrating that an MTS contains all information for mitochondrial targeting and membrane translocation of proteins (Schatz and Dobberstein (1996) Science 271:1519-1526; Neupert (1997) Annu Rev Biochem 66:863-917; Voos et al., (1999) Biochem Biophys Acta 1422:235-254).

Accordingly, in some embodiments, an mRNA of the disclosure comprises an ORF encoding an MTS, wherein the MTS is operably linked to a mitochondrial protein and targets the mitochondrial protein to the mitochondria. In some embodiments, the MTS is operably linked to a non-mitochondrial protein and targets the non-mitochondrial protein to the mitochondria. In some embodiments, the MTS is operably linked to a heterologous protein and targets the heterologous protein to the mitochondria. In some embodiments the heterologous protein is a mitochondrial protein. In some embodiments, the heterologous protein is a non-mitochondrial protein.

MTSs are known or can be identified within a protein or nucleic acid sequence by a person of ordinary skill in the art. For example, a method to identify a MTS is described in Claras & Vincens (1996) Eur J Biochem (241):779-786 (1996), the content of which is herein incorporated by reference in its entirety. Computer software is available to the skilled person to identify the MTS of a given sequence. Illustrative software notably comprises the MitoProt® software, which is available e.g. on the web site of the Institut fur Humangenetik; Technische Universitat München, Germany, http://ihg.gsf.de/ihg/mitoprot.html (see also ftp://ftp.ens.fr/pub/molbio). The MitoProt® software calculates the N-terminal protein region that can support a Mitochondrial Targeting Sequence and the cleavage site. The identification of the N-terminal mitochondrial targeting peptide that is present within a protein gives a direct access to the nucleic acid sequence, i.e. to the MTS (e.g. by reading the corresponding positions in the nucleic acid sequence coding for said protein). Other software/computational tools for predicting and/or identifying putative mitochondrial presequences and cleavage sites in sequences available to one skilled in the art include, but are not limited to, MitoFates (http://mitf.cbrc.jp/MitoFates/cgi-bin/top.cgi; see e.g., Fukasawa et al., (2015) Mol Cell Proteomics 14(4):1113-1126), iPSORT (http://ipsort.hgc.jp/; see e.g., Bannai et al., (2002) Bioinformatics 18(2):298-305); and TargetP (http://www.cbs.dtu.dk/services/TargetP/; Emanuelsson et al., (2000) J Mol Biol 300:1005-1016).

An MTS according to the present disclosure is any peptide that directs, localizes, translocates, sorts, or otherwise delivers a polypeptide (e.g., a therapeutic polypeptide) to the mitochondria of a cell. In some embodiments, the MTS is derived from a mitochondrial protein. MitoCarta2.0 is an inventory of 1158 human and mouse genes encoding mitochondrial proteins (see e.g., Calvo et al., (2015) Nucleic Acids Res 44(Database issue):D1251-D1258; Pagliarini et al., (2008) Cell 134:112-123). In some aspects, a suitable MTS is derived from a mitochondrial protein described in the MitoCarta2.0 mitochondrial protein inventory. Suitable MTSs encompass both naturally-occurring sequences and modified sequences that retain mitochondrial targeting ability and can be produced using recombinant and synthetic methods or purified from natural sources. In some aspects, the MTS is derived from a mitochondrial protein from any organism, including, but not limited to a mouse, a human, and a fungi (e.g., yeast). In some embodiments, the MTS is a fragment or variant of an MTS from a mouse, human, or yeast polypeptide. In some embodiments, the MTS comprises an amino acid sequence that is about 40%, about 45%, about 50%, about 55%, about 60%, about 65%, about 70%, about 75%, about 80%, about 85%, about 90%, about 95%, about 97%, about 99%, about 100% or 100% identical to an MTS from a mouse, human or yeast polypeptide.

In some aspects, the disclosure provides mRNA comprising an ORF encoding an MTS and a polypeptide, wherein the MTS is operably linked to the polypeptide. In some embodiments, the MTS is heterologous to the polypeptide. In some embodiments, the MTS is not heterologous to the polypeptide. In some embodiments, the MTS is located at the N-terminus of the polypeptide. In some embodiments, the MTS is fused to the polypeptide at the N-terminus.

In one embodiment, the MTS is operably linked to a protein not normally targeted to the mitochondria. In another embodiment, the MTS is operably linked to a protein that is a nuclear encoded mitochondrial protein.

In some embodiments, the MTS increases an expression level of a polypeptide translated from the mRNA relative to an mRNA that does not comprise the MTS. In some embodiments, the MTS increases an activity of a polypeptide translated from the mRNA relative to an mRNA that does not comprise the MTS. In some embodiments, the MTS increases an expression level and an activity of a polypeptide translated from the mRNA relative to an mRNA that does not comprise the MTS. In some embodiments, the expression level and/or activity is increased by at least about 1-fold, about 2-fold, about 3-fold, about 4-fold, about 5-fold, about 6-fold, about 7-fold, about 8-fold, about 9-fold, about 10-fold or more. In some embodiments, the expression level and/or activity is increased by about 1%, about 2%, about 3%, about 4%, about 5%, about 6%, about 7%, about 8%, about 9%, about 10%, about 15% about 20%, about 30%, about 40%, about 50%, about 60%, about 70%, about 80%, about 90%, or about 100%.

mRNA Construct Components

An mRNA may be a naturally or non-naturally occurring mRNA. An mRNA may include one or more modified nucleobases, nucleosides, or nucleotides, as described below, in which case it may be referred to as a “modified mRNA” or “mmRNA.” As described herein “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group.

An mRNA may include a 5′ untranslated region (5′UTR), a 3′ untranslated region (3′UTR), and/or a coding region (e.g., an open reading frame). In some embodiments, an mRNA provided by the disclosure comprises a 5′ UTR comprising a nucleotide sequence selected from the group consisting of: SEQ ID NO: 45 or SEQ ID NO: 4, or any 5′ UTR referred to by sequence in Table 9. In some embodiments, an mRNA provided by the disclosure comprises a 5′ UTR comprising a nucleotide sequence that is at least 50%, at least 60%, at least 70%, at least 80%, at least 85%, at least 90%, at least 95%, at least 98%, at least 99%, or 100% identical to a nucleotide sequence selected from the group consisting of: SEQ ID NO: 45 or SEQ ID NO: 4, or any 5′ UTR referred to by sequence in Table 9.

In some embodiments, an mRNA of the disclosure comprises a 5′UTR wherein the 5′UTR comprises one or more RNA elements. In some embodiments, the 5′UTR comprises a structural RNA element comprising a stem-loop comprising a nucleotide sequence selected from the group consisting of: SEQ ID NO: 6 or SEQ ID NO: 47. In some embodiments, the 5′UTR comprises a structural RNA element comprising a stem-loop comprising a nucleotide sequence identified by SEQ ID NO: 6. In some embodiments, the 5′UTR comprises the nucleotide sequence of SEQ ID NO: 116. In some embodiments, the 5′UTR comprises the nucleotide sequence of SEQ ID NO: 120. In some embodiments, the 5′UTR comprises the nucleotide sequence of SEQ ID NO: 124.

In some embodiments, an mRNA provided by the disclosure comprises a 3′UTR comprising a nucleotide sequence selected from a group consisting of: SEQ ID NO: 150, SEQ ID NO: 70, or any 3′UTR referred to by sequence in Table 10. In some embodiments, an mRNA provided by the disclosure comprises a 3′ UTR comprising a nucleotide sequence that is at least 50%, at least 60%, at least 70%, at least 80%, at least 85%, at least 90%, at least 95%, at least 98%, at least 99%, or 100% identical to a nucleotide sequence selected from the group consisting of: SEQ ID NO: 150, SEQ ID NO: 70, or any 3′UTR referred to by sequence in Table 10. In some embodiments, an mRNA provided by the disclosure comprises a 3′UTR comprising a nucleotide sequence of a 3′UTR derived from a naturally-occurring mRNA encoding a NEMP, or a fragment or variant thereof. In some embodiments, the 3′UTR comprises a nucleotide sequence selected from a group consisting of: SEQ ID NO: 72, SEQ ID NO: 74, SEQ ID NO: 76, SEQ ID NO: 78. In some embodiments, an mRNA provided by the disclosure comprises a 3′ UTR comprising a nucleotide sequence that is at least 50%, at least 60%, at least 70%, at least 80%, at least 85%, at least 90%, at least 95%, at least 98%, at least 99%, or 100% identical to a nucleotide sequence selected from the group consisting of: SEQ ID NO: 72, SEQ ID NO: 74, SEQ ID NO: 76, SEQ ID NO: 78.

An mRNA may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified.

In some embodiments, an mRNA as described herein may include a 5′ cap structure, a chain terminating nucleotide, optionally a Kozak sequence (also known as a Kozak consensus sequence), a stem loop, a polyA sequence, and/or a polyadenylation signal.

A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m⁷G(5′)ppp(5′)G, commonly written as m⁷GpppG. A cap species may also be an anti-reverse cap analog. A non-limiting list of possible cap species includes m⁷GpppG, m⁷Gpppm⁷G, m⁷3′dGpppG, m₂^{7,O3}′GpppG, m₂^{7,O3}′GppppG, m₂^{7,O2}′GppppG, m⁷Gpppm⁷G, m₂^{7,O3}′dGpppG, m₂^{7,O3}′GpppG, m₂^{7,O3}′GppppG, and m₂^{7,O2}′GppppG.

An mRNA may instead or additionally include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine. In some embodiments, incorporation of a chain terminating nucleotide into an mRNA, for example at the 3′-terminus, may result in stabilization of the mRNA, as described, for example, in International Patent Publication No. WO 2013/103659.

An mRNA may instead or additionally include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. In some embodiments, a stem loop may affect one or more function(s) of an mRNA, such as initiation of translation, translation efficiency, and/or transcriptional termination.

An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. In some embodiments, a polyA sequence may affect the nuclear export, translation, and/or stability of an mRNA.

An mRNA may instead or additionally include a microRNA binding site.

In some embodiments, an mRNA is a bicistronic mRNA comprising a first coding region and a second coding region with an intervening sequence comprising an internal ribosome entry site (IRES) sequence that allows for internal translation initiation between the first and second coding regions, or with an intervening sequence encoding a self-cleaving peptide, such as a 2A peptide. IRES sequences and 2A peptides are typically used to enhance expression of multiple proteins from the same vector. A variety of IRES sequences are known and available in the art and may be used, including, e.g., the encephalomyocarditis virus IRES.

### 5′UTR and Translation Initiation

In certain embodiments, the polynucleotide (e.g., mRNA) encoding a polypeptide of the present disclosure comprises a 5′ UTR and/or a translation initiation sequence. Natural 5′ UTRs comprise sequences involved in translation initiation. For example, Kozak sequences comprise natural 5′ UTRs and are commonly known to be involved in the process by which the ribosome initiates translation of many genes. 5′ UTRs also have been known to form secondary structures which are involved in elongation factor binding.

By engineering the features typically found in abundantly expressed genes of specific target organs, one can enhance the stability and protein production of the polynucleotides of the disclosure. For example, introduction of 5′ UTR of mRNA known to be upregulated in cancers, such as c-myc, could be used to enhance expression of a nucleic acid molecule, such as a polynucleotide, in cancer cells. Untranslated regions useful in the design and manufacture of polynucleotides include, but are not limited, to those disclosed in International Patent Publication No. WO 2014/164253 (see also US20160022840).

In addition to the exemplary 5′UTRs of the disclosure described herein, additional exemplary 5′UTRs useful to the disclosure are shown in Table 9. Variants of 5′ UTRs can be utilized wherein one or more nucleotides are added or removed to the termini, including A, U, C or G.

TABLE 9
Exemplary 5′-UTRs

| | | SEQ |
|---|---|---|
| 5′UTR | | ID |
| Identifier | Sequence | NO. |
| 5′v1.0 | GGGAAAUAAGAGAGAAAAGAAGAGUAAG | 45 |
| | AAGAAAUAUAAGAGCCACC | |
| 5′v1.0 | AGGAAAUAAGAGAGAAAAGAAGAGUAAG | 59 |
| A-start | AAGAAAUAUAAGAGCCACC | |
| 5′v1.0 | UAAGAGAGAAAAGAAGAGUAAGAAGAAA | 57 |
| Minus Leader | UAUAAGAGCCACC | |
| 5′v1.1 | GGGAAAUAAGAGAGAAAAGAAGAGUAAG | 4 |
| | AAGAAAUAUAAGACCCCGGCGCCGCCAC | |
| | C | |
| 5′v1.1 | AGGAAAUAAGAGAGAAAAGAAGAGUAAG | 60 |
| A-start | AAGAAAUAUAAGACCCCGGCGCCGCCAC | |
| | C | |
| 5′v1.1 | UAAGAGAGAAAAGAAGAGUAAGAAGAAA | 61 |
| minus leader | UAUAAGACCCCGGCGCCGCCACC | |
| 5UTR-001 | GGGAGAUCAGAGAGAAAAGAAGAGUAAG | 201 |
| | AAGAAAUAUAAGAGCCACC | |
| 5′UTR-002 | GGAAUAAAAGUCUCAACACAACAUAUAC | 202 |
| | AAAACAAACGAAUCUCAAGCAAUCAAGC | |
| | AUUCUACUUCUAUUGCAGCAAUUUAAAU | |
| | CAUUUCUUUUAAAGCAAAAGCAAUUUUC | |
| | UGAAAAUUUUCACCAUUUACGAACGAUA | |
| | GCAAC | |
| 5′UTR-003 | GGGAGACAAGCUUGGCAUUCCGGUACUG | 203 |
| | UUGGUAAAGCCACC | |
| 5′UTR-004 | GGGAAUUAACAGAGAAAAGAAGAGUAAG | 204 |
| | AAGAAAUAUAAGAGCCACC | |
| 5′UTR-005 | GGGAAAUUAGACAGAAAAGAAGAGUAAG | 205 |
| | AAGAAAUAUAAGAGCCACC | |
| 5′UTR-006 | GGGAAAUAAGAGAGUAAAGAACAGUAAG | 206 |
| | AAGAAAUAUAAGAGCCACC | |
| 5′UTR-007 | GGGAAAAAAGAGAGAAAAGAAGACUAAG | 207 |
| | AAGAAAUAUAAGAGCCACC | |
| 5′UTR-008 | GGGAAAUAAGAGAGAAAAGAAGAGUAAG | 208 |
| | AAGAUAUAUAAGAGCCACC | |
| 5′UTR-009 | GGGAAAUAAGAGACAAAACAAGAGUAAG | 209 |
| | AAGAAAUAUAAGAGCCACC | |
| 5′UTR-010 | GGGAAAUUAGAGAGUAAAGAACAGUAAG | 210 |
| | UAGAAUUAAAAGAGCCACC | |
| 5′UTR-011 | GGGAAAUAAGAGAGAAUAGAAGAGUAAG | 211 |
| | AAGAAAUAUAAGAGCCACC | |
| 5′UTR-012 | GGGAAAUAAGAGAGAAAAGAAGAGUAAG | 212 |
| | AAGAAAAUUAAGAGCCACC | |
| 5′UTR-013 | GGGAAAUAAGAGAGAAAAGAAGAGUAAG | 213 |
| | AAGAAAUUUAAGAGCCACC | |
| 5′UTR-014 | UCAAGCUUUUGGACCCUCGUACAGAAGC | 214 |
| | UAAUACGACUCACUAUAGGGAAAUAAGA | |
| | GAGAAAAGAAGAGUAAGAAGAAAUAUAA | |
| | GAGCCACC | |
| 5′UTR-015 | GGACAGAUCGCCUGGAGACGCCAUCCAC | 215 |
| | GCUGUUUUGACCUCCAUAGAAGACACCG | |
| | GGACCGAUCCAGCCUCCGCGGCCGGGAAC | |
| | GGUGCAUUGGAACGCGGAUUCCCCGUGC | |
| | CAAGAGUGACUCACCGUCCUUGACACG | |
| 5′UTR-016 | GGCGCUGCCUACGGAGGUGGCAGCCAUC | 216 |
| | UCCUUCUCGGCAUC | |
| S065 core | CCUCAUAUCCAGGCUCAAGAAUAGAGCU | 46 |
| | CAGUGUUUUGUUGUUUAAUCAUUCCGAC | |
| | GUGUUUUGCGAUAUUCGCGCAAAGCAGC | |
| | CAGUCGCGCGCUUGCUUUUAAGUAGAGU | |
| | UGUUUUUCCACCCGUUUGCCAGGCAUCU | |
| | UUAAUUUAACAUAUUUUUAUUUUUCAGG | |
| | CUAACCUA | |
| S065 | GGGAGACCUCAUAUCCAGGCUCAAGAAU | 42 |
| | AGAGCUCAGUGUUUUGUUGUUUAAUCAU | |
| | UCCGACGUGUUUUGCGAUAUUCGCGCAA | |
| | AGCAGCCAGUCGCGCGCUUGCUUUUAAG | |
| | UAGAGUUGUUUUUCCACCCGUUUGCCAG | |
| | GCAUCUUUAAUUUAACAUAUUUUUAUUU | |
| | UUCAGGCUAACCUAAAGCAGAGAA | |
| combo3_S065 | GGGAGACCUCAUAUCCAGGCUCAAGAAU | 39 |
| (S065 | AGAGCUCAGUGUUUUGUUGUUUAAUCAU | |
| ExtKozak) | UCCGACGUGUUUUGCGAUAUUCGCGCAA | |
| | AGCAGCCAGUCGCGCGCUUGCUUUUAAG | |
| | UAGAGUUGUUUUUCCACCCGUUUGCCAG | |
| | GCAUCUUUAAUUUAACAUAUUUUUAUUU | |
| | UUCAGGCUAACCUACGCCGCCACC | |

Other non-UTR sequences can also be used as regions or subregions within the polynucleotides. For example, introns or portions of introns sequences can be incorporated into regions of the polynucleotides. Incorporation of intronic sequences can increase protein production as well as polynucleotide levels.

Combinations of features can be included in flanking regions and can be contained within other features. For example, the ORF can be flanked by a 5′ UTR which can contain a strong Kozak translational initiation signal and/or a 3′ UTR which can include an oligo(dT) sequence for templated addition of a poly-A tail. A 5′ UTR can comprise a first polynucleotide fragment and a second polynucleotide fragment from the same and/or different genes such as the 5′ UTRs described in U.S. Patent Application Publication No. 2010-0293625.

These UTRs or portions thereof can be placed in the same orientation as in the transcript from which they were selected or can be altered in orientation or location. Hence a 5′ or 3′ UTR can be inverted, shortened, lengthened, made with one or more other 5′ UTRs or 3′ UTRs.

In some embodiments, the UTR sequences can be changed in some way in relation to a reference sequence. For example, a 3′ or 5′ UTR can be altered relative to a wild type or native UTR by the change in orientation or location as taught above or can be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ or 3′ UTR can be used. As used herein, a “double” UTR is one in which two copies of the same UTR are encoded either in series or substantially in series. For example, a double beta-globin 3′ UTR can be used as described in U.S. Patent Application Publication No. 2010-0129877.

In some embodiments, flanking regions can be heterologous. In some embodiments, the 5′ untranslated region can be derived from a different species than the 3′ untranslated region. The untranslated region can also include translation enhancer elements (TEE). As a non-limiting example, the TEE can include those described in U.S. Patent Application Publication No. 2009-0226470.

In some embodiments, the mRNAs provided by the disclosure comprise a 5′ UTR comprising a T7 leader sequence at the 5′ end of the 5′ UTR. In some embodiments, the mRNA of the disclosure comprises a 5′ UTR comprising a T7 leader sequence comprising the sequence GGGAGA at the 5′ end of the 5′ UTR. In some embodiments, the mRNA of the disclosure comprises a 5′ UTR comprising a T7 leader sequence comprising the sequence GGGAAA at the 5′ end of the 5′ UTR. In some embodiments, the mRNA comprises a 5′ UTR which does not comprise a T7 leader sequence at the 5′ end of the 5′ UTR. In another aspect, the disclosure provides an mRNA comprising a 5′ UTR, wherein the nucleotide sequence of the 5′ UTR comprises any one of the nucleotide sequences set forth in Table 9.

### 3′UTR and AU Rich Elements

In certain embodiments, the polynucleotide (e.g., mRNA) encoding a polypeptide further comprises a 3′UTR. 3′UTR is the section of mRNA that immediately follows the translation termination codon and often contains regulatory regions that post-transcriptionally influence gene expression. Regulatory regions within the 3′UTR can influence polyadenylation, translation efficiency, localization, and stability of the mRNA. In one embodiment, the 3′UTR useful for the disclosure comprises a binding site for regulatory proteins or microRNAs. In some embodiments, the 3′-UTR has a silencer region, which binds to repressor proteins and inhibits the expression of the mRNA. In other embodiments, the 3′UTR comprises an AU-rich element. Proteins bind AREs to affect the stability or decay rate of transcripts in a localized manner or affect translation initiation. In other embodiments, the 3′UTR comprises the sequence AAUAAA that directs addition of several hundred adenine residues called the poly(A) tail to the end of the mRNA transcript.

In addition to the exemplary 3′UTRs of the disclosure described herein, additional exemplary 3′UTRs useful to the disclosure are shown in Table 10. Variants of 3′ UTRs can be utilized wherein one or more nucleotides are added or removed to the termini, including A, U, C or G.

TABLE 10
Exemplary 3′-Untranslated Regions

| 3′UTR | Name/ | | |
|---|---|---|---|
| Identifier | Description | Sequence | SEQ ID NO. |
| Downstream | V1.0 3′UTR | UGAUAAUAGGCUGGAGCCUCGGUGGCCA | 150 |
| UTR | | UGCUUCUUGCCCCUUGGGCCUCCCCCCAG | |
| | | CCCCUCCUCCCCUUCCUGCACCCGUACCC | |
| | | CCGUGGUCUUUGAAUAAAGUCUGAGUGG | |
| | | GCGGC | |
| Downstream | v1.1 3′UTR | UGAUAAUAGGCUGGAGCCUCGGUGGCCU | 70 |
| UTR | | AGCUUCUUGCCCCUUGGGCCUCCCCCCAG | |
| | | CCCCUCCUCCCCUUCCUGCACCCGUACCC | |
| | | CCGUGGUCUUUGAAUAAAGUCUGAGUGG | |
| | | GCGGC | |
| 3′ UTR-001 | Creatine | GCGCCUGCCCACCUGCCACCGACUGCUGG | 217 |
| | Kinase | AACCCAGCCAGUGGGAGGGCCUGGCCCAC | |
| | | CAGAGUCCUGCUCCCUCACUCCUCGCCCC | |
| | | GCCCCCUGUCCCAGAGUCCCACCUGGGGG | |
| | | CUCUCUCCACCCUUCUCAGAGUUCCAGUU | |
| | | UCAACCAGAGUUCCAACCAAUGGGCUCCA | |
| | | UCCUCUGGAUUCUGGCCAAUGAAAUAUC | |
| | | UCCCUGGCAGGGUCCUCUUCUUUUCCCAG | |
| | | AGCUCCACCCCAACCAGGAGCUCUAGUUA | |
| | | AUGGAGAGCUCCCAGCACACUCGGAGCU | |
| | | UGUGCUUUGUCUCCACGCAAAGCGAUAA | |
| | | AUAAAAGCAUUGGUGGCCUUUGGUCUUU | |
| | | GAAUAAAGCCUGAGUAGGAAGUCUAGA | |
| 3′ UTR-002 | Myoglobin | GCCCCUGCCGCUCCCACCCCCACCCAUCU | 218 |
| | | GGGCCCCGGGUUCAAGAGAGAGCGGGGU | |
| | | CUGAUCUCGUGUAGCCAUAUAGAGUUUG | |
| | | CUUCUGAGUGUCUGCUUUGUUUAGUAGA | |
| | | GGUGGGCAGGAGGAGCUGAGGGGCUGGG | |
| | | GCUGGGGUGUUGAAGUUGGCUUUGCAUG | |
| | | CCCAGCGAUGCGCCUCCCUGUGGGAUGUC | |
| | | AUCACCCUGGGAACCGGGAGUGGCCCUU | |
| | | GGCUCACUGUGUUCUGCAUGGUUUGGAU | |
| | | CUGAAUUAAUUGUCCUUUCUUCUAAAUC | |
| | | CCAACCGAACUUCUUCCAACCUCCAAACU | |
| | | GGCUGUAACCCCAAAUCCAAGCCAUUAAC | |
| | | UACACCUGACAGUAGCAAUUGUCUGAUU | |
| | | AAUCACUGGCCCCUUGAAGACAGCAGAA | |
| | | UGUCCCUUUGCAAUGAGGAGGAGAUCUG | |
| | | GGCUGGGCGGGCCAGCUGGGGAAGCAUU | |
| | | UGACUAUCUGGAACUUGUGUGUGCCUCC | |
| | | UCAGGUAUGGCAGUGACUCACCUGGUUU | |
| | | UAAUAAAACAACCUGCAACAUCUCAUGG | |
| | | UCUUUGAAUAAAGCCUGAGUAGGAAGUC | |
| | | UAGA | |
| 3′ UTR-003 | α-actin | ACACACUCCACCUCCAGCACGCGACUUCU | 219 |
| | | CAGGACGACGAAUCUUCUCAAUGGGGGG | |
| | | GCGGCUGAGCUCCAGCCACCCCGCAGUCA | |
| | | CUUUCUUUGUAACAACUUCCGUUGCUGC | |
| | | CAUCGUAAACUGACACAGUGUUUAUAAC | |
| | | GUGUACAUACAUUAACUUAUUACCUCAU | |
| | | UUUGUUAUUUUUCGAAACAAAGCCCUGU | |
| | | GGAAGAAAAUGGAAAACUUGAAGAAGCA | |
| | | UUAAAGUCAUUCUGUUAAGCUGCGUAAA | |
| | | UGGUCUUUGAAUAAAGCCUGAGUAGGAA | |
| | | GUCUAGA | |
| 3′ UTR-004 | Albumin | CAUCACAUUUAAAAGCAUCUCAGCCUACC | 220 |
| | | AUGAGAAUAAGAGAAAGAAAAUGAAGAU | |
| | | CAAAAGCUUAUUCAUCUGUUUUUCUUUU | |
| | | UCGUUGGUGUAAAGCCAACACCCUGUCU | |
| | | AAAAAACAUAAAUUUCUUUAAUCAUUUU | |
| | | GCCUCUUUUCUCUGUGCUUCAAUUAAUA | |
| | | AAAAAUGGAAAGAAUCUAAUAGAGUGGU | |
| | | ACAGCACUGUUAUUUUUCAAAGAUGUGU | |
| | | UGCUAUCCUGAAAAUUCUGUAGGUUCUG | |
| | | UGGAAGUUCCAGUGUUCUCUCUUAUUCC | |
| | | ACUUCGGUAGAGGAUUUCUAGUUUCUUG | |
| | | UGGGCUAAUUAAAUAAAUCAUUAAUACU | |
| | | CUUCUAAUGGUCUUUGAAUAAAGCCUGA | |
| | | GUAGGAAGUCUAGA | |
| 3′ UTR-005 | α-globin | GCUGCCUUCUGCGGGGCUUGCCUUCUGGC | 221 |
| | | CAUGCCCUUCUUCUCUCCCUUGCACCUGU | |
| | | ACCUCUUGGUCUUUGAAUAAAGCCUGAG | |
| | | UAGGAAGGCGGCCGCUCGAGCAUGCAUC | |
| | | UAGA | |
| 3′ UTR-006 | G-CSF | GCCAAGCCCUCCCCAUCCCAUGUAUUUAU | 222 |
| | | CUCUAUUUAAUAUUUAUGUCUAUUUAAG | |
| | | CCUCAUAUUUAAAGACAGGGAAGAGCAG | |
| | | AACGGAGCCCCAGGCCUCUGUGUCCUUCC | |
| | | CUGCAUUUCUGAGUUUCAUUCUCCUGCC | |
| | | UGUAGCAGUGAGAAAAAGCUCCUGUCCU | |
| | | CCCAUCCCCUGGACUGGGAGGUAGAUAG | |
| | | GUAAAUACCAAGUAUUUAUUACUAUGAC | |
| | | UGCUCCCCAGCCCUGGCUCUGCAAUGGGC | |
| | | ACUGGGAUGAGCCGCUGUGAGCCCCUGG | |
| | | UCCUGAGGGUCCCCACCUGGGACCCUUGA | |
| | | GAGUAUCAGGUCUCCCACGUGGGAGACA | |
| | | AGAAAUCCCUGUUUAAUAUUUAAACAGC | |
| | | AGUGUUCCCCAUCUGGGUCCUUGCACCCC | |
| | | UCACUCUGGCCUCAGCCGACUGCACAGCG | |
| | | GCCCCUGCAUCCCCUUGGCUGUGAGGCCC | |
| | | CUGGACAAGCAGAGGUGGCCAGAGCUGG | |
| | | GAGGCAUGGCCCUGGGGUCCCACGAAUU | |
| | | UGCUGGGGAAUCUCGUUUUUCUUCUUAA | |
| | | GACUUUUGGGACAUGGUUUGACUCCCGA | |
| | | ACAUCACCGACGCGUCUCCUGUUUUUCUG | |
| | | GGUGGCCUCGGGACACCUGCCCUGCCCCC | |
| | | ACGAGGGUCAGGACUGUGACUCUUUUUA | |
| | | GGGCCAGGCAGGUGCCUGGACAUUUGCC | |
| | | UUGCUGGACGGGGACUGGGGAUGUGGGA | |
| | | GGGAGCAGACAGGAGGAAUCAUGUCAGG | |
| | | CCUGUGUGUGAAAGGAAGCUCCACUGUC | |
| | | ACCCUCCACCUCUUCACCCCCCACUCACC | |
| | | AGUGUCCCCUCCACUGUCACAUUGUAACU | |
| | | GAACUUCAGGAUAAUAAAGUGUUUGCCU | |
| | | CCAUGGUCUUUGAAUAAAGCCUGAGUAG | |
| | | GAAGGCGGCCGCUCGAGCAUGCAUCUAG | |
| | | A | |
| 3′ UTR-007 | Col1a2; | ACUCAAUCUAAAUUAAAAAAGAAAGAAA | 223 |
| | collagen, | UUUGAAAAAACUUUCUCUUUGCCAUUUC | |
| | type I, | UUCUUCUUCUUUUUUAACUGAAAGCUGA | |
| | alpha 2 | AUCCUUCCAUUUCUUCUGCACAUCUACUU | |
| | | GCUUAAAUUGUGGGCAAAAGAGAAAAAG | |
| | | AAGGAUUGAUCAGAGCAUUGUGCAAUAC | |
| | | AGUUUCAUUAACUCCUUCCCCCGCUCCCC | |
| | | CAAAAAUUUGAAUUUUUUUUUCAACACU | |
| | | CUUACACCUGUUAUGGAAAAUGUCAACC | |
| | | UUUGUAAGAAAACCAAAAUAAAAAUUGA | |
| | | AAAAUAAAAACCAUAAACAUUUGCACCA | |
| | | CUUGUGGCUUUUGAAUAUCUUCCACAGA | |
| | | GGGAAGUUUAAAACCCAAACUUCCAAAG | |
| | | GUUUAAACUACCUCAAAACACUUUCCCA | |
| | | UGAGUGUGAUCCACAUUGUUAGGUGCUG | |
| | | ACCUAGACAGAGAUGAACUGAGGUCCUU | |
| | | GUUUUGUUUUGUUCAUAAUACAAAGGUG | |
| | | CUAAUUAAUAGUAUUUCAGAUACUUGAA | |
| | | GAAUGUUGAUGGUGCUAGAAGAAUUUGA | |
| | | GAAGAAAUACUCCUGUAUUGAGUUGUAU | |
| | | CGUGUGGUGUAUUUUUUAAAAAAUUUGA | |
| | | UUUAGCAUUCAUAUUUUCCAUCUUAUUC | |
| | | CCAAUUAAAAGUAUGCAGAUUAUUUGCC | |
| | | CAAAUCUUCUUCAGAUUCAGCAUUUGUU | |
| | | CUUUGCCAGUCUCAUUUUCAUCUUCUUCC | |
| | | AUGGUUCCACAGAAGCUUUGUUUCUUGG | |
| | | GCAAGCAGAAAAAUUAAAUUGUACCUAU | |
| | | UUUGUAUAUGUGAGAUGUUUAAAUAAAU | |
| | | UGUGAAAAAAAUGAAAUAAAGCAUGUUU | |
| | | GGUUUUCCAAAAGAACAUAU | |
| 3′ UTR-008 | Col6a2; | CGCCGCCGCCCGGGCCCCGCAGUCGAGGG | 224 |
| | collagen, | UCGUGAGCCCACCCCGUCCAUGGUGCUAA | |
| | type VI, | GCGGGCCCGGGUCCCACACGGCCAGCACC | |
| | alpha 2 | GCUGCUCACUCGGACGACGCCCUGGGCCU | |
| | | GCACCUCUCCAGCUCCUCCCACGGGGUCC | |
| | | CCGUAGCCCCGGCCCCCGCCCAGCCCCAG | |
| | | GUCUCCCCAGGCCCUCCGCAGGCUGCCCG | |
| | | GCCUCCCUCCCCCUGCAGCCAUCCCAAGG | |
| | | CUCCUGACCUACCUGGCCCCUGAGCUCUG | |
| | | GAGCAAGCCCUGACCCAAUAAAGGCUUU | |
| | | GAACCCAU | |
| 3′ UTR-009 | RPN1; | GGGGCUAGAGCCCUCUCCGCACAGCGUGG | 225 |
| | ribophorin I | AGACGGGGCAAGGAGGGGGGUUAUUAGG | |
| | | AUUGGUGGUUUUGUUUUGCUUUGUUUAA | |
| | | AGCCGUGGGAAAAUGGCACAACUUUACC | |
| | | UCUGUGGGAGAUGCAACACUGAGAGCCA | |
| | | AGGGGUGGGAGUUGGGAUAAUUUUUAUA | |
| | | UAAAAGAAGUUUUUCCACUUUGAAUUGC | |
| | | UAAAAGUGGCAUUUUUCCUAUGUGCAGU | |
| | | CACUCCUCUCAUUUCUAAAAUAGGGACG | |
| | | UGGCCAGGCACGGUGGCUCAUGCCUGUA | |
| | | AUCCCAGCACUUUGGGAGGCCGAGGCAG | |
| | | GCGGCUCACGAGGUCAGGAGAUCGAGAC | |
| | | UAUCCUGGCUAACACGGUAAAACCCUGU | |
| | | CUCUACUAAAAGUACAAAAAAUUAGCUG | |
| | | GGCGUGGUGGUGGGCACCUGUAGUCCCA | |
| | | GCUACUCGGGAGGCUGAGGCAGGAGAAA | |
| | | GGCAUGAAUCCAAGAGGCAGAGCUUGCA | |
| | | GUGAGCUGAGAUCACGCCAUUGCACUCC | |
| | | AGCCUGGGCAACAGUGUUAAGACUCUGU | |
| | | CUCAAAUAUAAAUAAAUAAAUAAAUAAA | |
| | | UAAAUAAAUAAAUAAAAAUAAAGCGAGA | |
| | | UGUUGCCCUCAAA | |
| 3′ UTR-010 | LRP1; low | GGCCCUGCCCCGUCGGACUGCCCCCAGAA | 226 |
| | density | AGCCUCCUGCCCCCUGCCAGUGAAGUCCU | |
| | lipoprotein | UCAGUGAGCCCCUCCCCAGCCAGCCCUUC | |
| | receptor- | CCUGGCCCCGCCGGAUGUAUAAAUGUAA | |
| | related | AAAUGAAGGAAUUACAUUUUAUAUGUGA | |
| | protein 1 | GCGAGCAAGCCGGCAAGCGAGCACAGUA | |
| | | UUAUUUCUCCAUCCCCUCCCUGCCUGCUC | |
| | | CUUGGCACCCCCAUGCUGCCUUCAGGGAG | |
| | | ACAGGCAGGGAGGGCUUGGGGCUGCACC | |
| | | UCCUACCCUCCCACCAGAACGCACCCCAC | |
| | | UGGGAGAGCUGGUGGUGCAGCCUUCCCC | |
| | | UCCCUGUAUAAGACACUUUGCCAAGGCU | |
| | | CUCCCCUCUCGCCCCAUCCCUGCUUGCCC | |
| | | GCUCCCACAGCUUCCUGAGGGCUAAUUCU | |
| | | GGGAAGGGAGAGUUCUUUGCUGCCCCUG | |
| | | UCUGGAAGACGUGGCUCUGGGUGAGGUA | |
| | | GGCGGGAAAGGAUGGAGUGUUUUAGUUC | |
| | | UUGGGGGAGGCCACCCCAAACCCCAGCCC | |
| | | CAACUCCAGGGGCACCUAUGAGAUGGCC | |
| | | AUGCUCAACCCCCCUCCCAGACAGGCCCU | |
| | | CCCUGUCUCCAGGGCCCCCACCGAGGUUC | |
| | | CCAGGGCUGGAGACUUCCUCUGGUAAAC | |
| | | AUUCCUCCAGCCUCCCCUCCCCUGGGGAC | |
| | | GCCAAGGAGGUGGGCCACACCCAGGAAG | |
| | | GGAAAGCGGGCAGCCCCGUUUUGGGGAC | |
| | | GUGAACGUUUUAAUAAUUUUUGCUGAAU | |
| | | UCCUUUACAACUAAAUAACACAGAUAUU | |
| | | GUUAUAAAUAAAAUUGU | |
| 3′UTR-011 | Nnt1; | AUAUUAAGGAUCAAGCUGUUAGCUAAUA | 227 |
| | cardio- | AUGCCACCUCUGCAGUUUUGGGAACAGG | |
| | trophin- | CAAAUAAAGUAUCAGUAUACAUGGUGAU | |
| | like cytokine | GUACAUCUGUAGCAAAGCUCUUGGAGAA | |
| | factor 1 | AAUGAAGACUGAAGAAAGCAAAGCAAAA | |
| | | ACUGUAUAGAGAGAUUUUUCAAAAGCAG | |
| | | UAAUCCCUCAAUUUUAAAAAAGGAUUGA | |
| | | AAAUUCUAAAUGUCUUUCUGUGCAUAUU | |
| | | UUUUGUGUUAGGAAUCAAAAGUAUUUUA | |
| | | UAAAAGGAGAAAGAACAGCCUCAUUUUA | |
| | | GAUGUAGUCCUGUUGGAUUUUUUAUGCC | |
| | | UCCUCAGUAACCAGAAAUGUUUUAAAAA | |
| | | ACUAAGUGUUUAGGAUUUCAAGACAACA | |
| | | UUAUACAUGGCUCUGAAAUAUCUGACAC | |
| | | AAUGUAAACAUUGCAGGCACCUGCAUUU | |
| | | UAUGUUUUUUUUUUCAACAAAUGUGACU | |
| | | AAUUUGAAACUUUUAUGAACUUCUGAGC | |
| | | UGUCCCCUUGCAAUUCAACCGCAGUUUG | |
| | | AAUUAAUCAUAUCAAAUCAGUUUUAAUU | |
| | | UUUUAAAUUGUACUUCAGAGUCUAUAUU | |
| | | UCAAGGGCACAUUUUCUCACUACUAUUU | |
| | | UAAUACAUUAAAGGACUAAAUAAUCUUU | |
| | | CAGAGAUGCUGGAAACAAAUCAUUUGCU | |
| | | UUAUAUGUUUCAUUAGAAUACCAAUGAA | |
| | | ACAUACAACUUGAAAAUUAGUAAUAGUA | |
| | | UUUUUGAAGAUCCCAUUUCUAAUUGGAG | |
| | | AUCUCUUUAAUUUCGAUCAACUUAUAAU | |
| | | GUGUAGUACUAUAUUAAGUGCACUUGAG | |
| | | UGGAAUUCAACAUUUGACUAAUAAAAUG | |
| | | AGUUCAUCAUGUUGGCAAGUGAUGUGGC | |
| | | AAUUAUCUCUGGUGACAAAAGAGUAAAA | |
| | | UCAAAUAUUUCUGCCUGUUACAAAUAUC | |
| | | AAGGAAGACCUGCUACUAUGAAAUAGAU | |
| | | GACAUUAAUCUGUCUUCACUGUUUAUAA | |
| | | UACGGAUGGAUUUUUUUUCAAAUCAGUG | |
| | | UGUGUUUUGAGGUCUUAUGUAAUUGAUG | |
| | | ACAUUUGAGAGAAAUGGUGGCUUUUUUU | |
| | | AGCUACCUCUUUGUUCAUUUAAGCACCA | |
| | | GUAAAGAUCAUGUCUUUUUAUAGAAGUG | |
| | | UAGAUUUUCUUUGUGACUUUGCUAUCGU | |
| | | GCCUAAAGCUCUAAAUAUAGGUGAAUGU | |
| | | GUGAUGAAUACUCAGAUUAUUUGUCUCU | |
| | | CUAUAUAAUUAGUUUGGUACUAAGUUUC | |
| | | UCAAAAAAUUAUUAACACAUGAAAGACA | |
| | | AUCUCUAAACCAGAAAAAGAAGUAGUAC | |
| | | AAAUUUUGUUACUGUAAUGCUCGCGUUU | |
| | | AGUGAGUUUAAAACACACAGUAUCUUUU | |
| | | GGUUUUAUAAUCAGUUUCUAUUUUGCUG | |
| | | UGCCUGAGAUUAAGAUCUGUGUAUGUGU | |
| | | GUGUGUGUGUGUGUGCGUUUGUGUGUUA | |
| | | AAGCAGAAAAGACUUUUUUAAAAGUUUU | |
| | | AAGUGAUAAAUGCAAUUUGUUAAUUGAU | |
| | | CUUAGAUCACUAGUAAACUCAGGGCUGA | |
| | | AUUAUACCAUGUAUAUUCUAUUAGAAGA | |
| | | AAGUAAACACCAUCUUUAUUCCUGCCCU | |
| | | UUUUCUUCUCUCAAAGUAGUUGUAGUUA | |
| | | UAUCUAGAAAGAAGCAAUUUUGAUUUCU | |
| | | UGAAAAGGUAGUUCCUGCACUCAGUUUA | |
| | | AACUAAAAAUAAUCAUACUUGGAUUUUA | |
| | | UUUAUUUUUGUCAUAGUAAAAAUUUUAA | |
| | | UUUAUAUAUAUUUUUAUUUAGUAUUAUC | |
| | | UUAUUCUUUGCUAUUUGCCAAUCCUUUG | |
| | | UCAUCAAUUGUGUUAAAUGAAUUGAAAA | |
| | | UUCAUGCCCUGUUCAUUUUAUUUUACUU | |
| | | UAUUGGUUAGGAUAUUUAAAGGAUUUUU | |
| | | GUAUAUAUAAUUUCUUAAAUUAAUAUUC | |
| | | CAAAAGGUUAGUGGACUUAGAUUAUAAA | |
| | | UUAUGGCAAAAAUCUAAAAACAACAAAA | |
| | | AUGAUUUUUAUACAUUCUAUUUCAUUAU | |
| | | UCCUCUUUUUCCAAUAAGUCAUACAAUU | |
| | | GGUAGAUAUGACUUAUUUUAUUUUUGUA | |
| | | UUAUUCACUAUAUCUUUAUGAUAUUUAA | |
| | | GUAUAAAUAAUUAAAAAAAUUUAUUGUA | |
| | | CCUUAUAGUCUGUCACCAAAAAAAAAAA | |
| | | AUUAUCUGUAGGUAGUGAAAUGCUAAUG | |
| | | UUGAUUUGUCUUUAAGGGCUUGUUAACU | |
| | | AUCCUUUAUUUUCUCAUUUGUCUUAAAU | |
| | | UAGGAGUUUGUGUUUAAAUUACUCAUCU | |
| | | AAGCAAAAAAUGUAUAUAAAUCCCAUUA | |
| | | CUGGGUAUAUACCCAAAGGAUUAUAAAU | |
| | | CAUGCUGCUAUAAAGACACAUGCACACG | |
| | | UAUGUUUAUUGCAGCACUAUUCACAAUA | |
| | | GCAAAGACUUGGAACCAACCCAAAUGUC | |
| | | CAUCAAUGAUAGACUUGAUUAAGAAAAU | |
| | | GUGCACAUAUACACCAUGGAAUACUAUG | |
| | | CAGCCAUAAAAAAGGAUGAGUUCAUGUC | |
| | | CUUUGUAGGGACAUGGAUAAAGCUGGAA | |
| | | ACCAUCAUUCUGAGCAAACUAUUGCAAG | |
| | | GACAGAAAACCAAACACUGCAUGUUCUC | |
| | | ACUCAUAGGUGGGAAUUGAACAAUGAGA | |
| | | ACACUUGGACACAAGGUGGGGAACACCA | |
| | | CACACCAGGGCCUGUCAUGGGGUGGGGG | |
| | | GAGUGGGGAGGGAUAGCAUUAGGAGAUA | |
| | | UACCUAAUGUAAAUGAUGAGUUAAUGGG | |
| | | UGCAGCACACCAACAUGGCACAUGUAUA | |
| | | CAUAUGUAGCAAACCUGCACGUUGUGCA | |
| | | CAUGUACCCUAGAACUUAAAGUAUAAUU | |
| | | AAAAAAAAAAAGAAAACAGAAGCUAUUU | |
| | | AUAAAGAAGUUAUUUGCUGAAAUAAAUG | |
| | | UGAUCUUUCCCAUUAAAAAAAUAAAGAA | |
| | | AUUUUGGGGUAAAAAAACACAAUAUAUU | |
| | | GUAUUCUUGAAAAAUUCUAAGAGAGUGG | |
| | | AUGUGAAGUGUUCUCACCACAAAAGUGA | |
| | | UAACUAAUUGAGGUAAUGCACAUAUUAA | |
| | | UUAGAAAGAUUUUGUCAUUCCACAAUGU | |
| | | AUAUAUACUUAAAAAUAUGUUAUACACA | |
| | | AUAAAUACAUACAUUAAAAAAUAAGUAA | |
| | | AUGUA | |
| 3′ UTR-012 | Col6a1; | CCCACCCUGCACGCCGGCACCAAACCCUG | 228 |
| | collagen, | UCCUCCCACCCCUCCCCACUCAUCACUAA | |
| | type VI, | ACAGAGUAAAAUGUGAUGCGAAUUUUCC | |
| | alpha 1 | CGACCAACCUGAUUCGCUAGAUUUUUUU | |
| | | UAAGGAAAAGCUUGGAAAGCCAGGACAC | |
| | | AACGCUGCUGCCUGCUUUGUGCAGGGUC | |
| | | CUCCGGGGCUCAGCCCUGAGUUGGCAUCA | |
| | | CCUGCGCAGGGCCCUCUGGGGCUCAGCCC | |
| | | UGAGCUAGUGUCACCUGCACAGGGCCCUC | |
| | | UGAGGCUCAGCCCUGAGCUGGCGUCACCU | |
| | | GUGCAGGGCCCUCUGGGGCUCAGCCCUGA | |
| | | GCUGGCCUCACCUGGGUUCCCCACCCCGG | |
| | | GCUCUCCUGCCCUGCCCUCCUGCCCGCCC | |
| | | UCCCUCCUGCCUGCGCAGCUCCUUCCCUA | |
| | | GGCACCUCUGUGCUGCAUCCCACCAGCCU | |
| | | GAGCAAGACGCCCUCUCGGGGCCUGUGCC | |
| | | GCACUAGCCUCCCUCUCCUCUGUCCCCAU | |
| | | AGCUGGUUUUUCCCACCAAUCCUCACCUA | |
| | | ACAGUUACUUUACAAUUAAACUCAAAGC | |
| | | AAGCUCUUCUCCUCAGCUUGGGGCAGCCA | |
| | | UUGGCCUCUGUCUCGUUUUGGGAAACCA | |
| | | AGGUCAGGAGGCCGUUGCAGACAUAAAU | |
| | | CUCGGCGACUCGGCCCCGUCUCCUGAGGG | |
| | | UCCUGCUGGUGACCGGCCUGGACCUUGGC | |
| | | CCUACAGCCCUGGAGGCCGCUGCUGACCA | |
| | | GCACUGACCCCGACCUCAGAGAGUACUCG | |
| | | CAGGGGCGCUGGCUGCACUCAAGACCCUC | |
| | | GAGAUUAACGGUGCUAACCCCGUCUGCU | |
| | | CCUCCCUCCCGCAGAGACUGGGGCCUGGA | |
| | | CUGGACAUGAGAGCCCCUUGGUGCCACA | |
| | | GAGGGCUGUGUCUUACUAGAAACAACGC | |
| | | AAACCUCUCCUUCCUCAGAAUAGUGAUG | |
| | | UGUUCGACGUUUUAUCAAAGGCCCCCUU | |
| | | UCUAUGUUCAUGUUAGUUUUGCUCCUUC | |
| | | UGUGUUUUUUUCUGAACCAUAUCCAUGU | |
| | | UGCUGACUUUUCCAAAUAAAGGUUUUCA | |
| | | CUCCUCUC | |
| 3′UTR-013 | Calr; | AGAGGCCUGCCUCCAGGGCUGGACUGAG | 229 |
| | calreticulin | GCCUGAGCGCUCCUGCCGCAGAGCUGGCC | |
| | | GCGCCAAAUAAUGUCUCUGUGAGACUCG | |
| | | AGAACUUUCAUUUUUUUCCAGGCUGGUU | |
| | | CGGAUUUGGGGUGGAUUUUGGUUUUGUU | |
| | | CCCCUCCUCCACUCUCCCCCACCCCCUCCC | |
| | | CGCCCUUUUUUUUUUUUUUUUUUAAACU | |
| | | GGUAUUUUAUCUUUGAUUCUCCUUCAGC | |
| | | CCUCACCCCUGGUUCUCAUCUUUCUUGAU | |
| | | CAACAUCUUUUCUUGCCUCUGUCCCCUUC | |
| | | UCUCAUCUCUUAGCUCCCCUCCAACCUGG | |
| | | GGGGCAGUGGUGUGGAGAAGCCACAGGC | |
| | | CUGAGAUUUCAUCUGCUCUCCUUCCUGG | |
| | | AGCCCAGAGGAGGGCAGCAGAAGGGGGU | |
| | | GGUGUCUCCAACCCCCCAGCACUGAGGAA | |
| | | GAACGGGGCUCUUCUCAUUUCACCCCUCC | |
| | | CUUUCUCCCCUGCCCCCAGGACUGGGCCA | |
| | | CUUCUGGGUGGGGCAGUGGGUCCCAGAU | |
| | | UGGCUCACACUGAGAAUGUAAGAACUAC | |
| | | AAACAAAAUUUCUAUUAAAUUAAAUUUU | |
| | | GUGUCUCC | |
| 3′ UTR-014 | Collal; | CUCCCUCCAUCCCAACCUGGCUCCCUCCC | 230 |
| | collagen, | ACCCAACCAACUUUCCCCCCAACCCGGAA | |
| | type I, | ACAGACAAGCAACCCAAACUGAACCCCCU | |
| | alpha 1 | CAAAAGCCAAAAAAUGGGAGACAAUUUC | |
| | | ACAUGGACUUUGGAAAAUAUUUUUUUCC | |
| | | UUUGCAUUCAUCUCUCAAACUUAGUUUU | |
| | | UAUCUUUGACCAACCGAACAUGACCAAA | |
| | | AACCAAAAGUGCAUUCAACCUUACCAAA | |
| | | AAAAAAAAAAAAAAAAGAAUAAAUAAAU | |
| | | AACUUUUUAAAAAAGGAAGCUUGGUCCA | |
| | | CUUGCUUGAAGACCCAUGCGGGGGUAAG | |
| | | UCCCUUUCUGCCCGUUGGGCUUAUGAAA | |
| | | CCCCAAUGCUGCCCUUUCUGCUCCUUUCU | |
| | | CCACACCCCCCUUGGGGCCUCCCCUCCAC | |
| | | UCCUUCCCAAAUCUGUCUCCCCAGAAGAC | |
| | | ACAGGAAACAAUGUAUUGUCUGCCCAGC | |
| | | AAUCAAAGGCAAUGCUCAAACACCCAAG | |
| | | UGGCCCCCACCCUCAGCCCGCUCCUGCCC | |
| | | GCCCAGCACCCCCAGGCCCUGGGGGACCU | |
| | | GGGGUUCUCAGACUGCCAAAGAAGCCUU | |
| | | GCCAUCUGGCGCUCCCAUGGCUCUUGCAA | |
| | | CAUCUCCCCUUCGUUUUUGAGGGGGUCA | |
| | | UGCCGGGGGAGCCACCAGCCCCUCACUGG | |
| | | GUUCGGAGGAGAGUCAGGAAGGGCCACG | |
| | | ACAAAGCAGAAACAUCGGAUUUGGGGAA | |
| | | CGCGUGUCAAUCCCUUGUGCCGCAGGGCU | |
| | | GGGCGGGAGAGACUGUUCUGUUCCUUGU | |
| | | GUAACUGUGUUGCUGAAAGACUACCUCG | |
| | | UUCUUGUCUUGAUGUGUCACCGGGGCAA | |
| | | CUGCCUGGGGGCGGGGAUGGGGGCAGGG | |
| | | UGGAAGCGGCUCCCCAUUUUAUACCAAA | |
| | | GGUGCUACAUCUAUGUGAUGGGUGGGGU | |
| | | GGGGAGGGAAUCACUGGUGCUAUAGAAA | |
| | | UUGAGAUGCCCCCCCAGGCCAGCAAAUGU | |
| | | UCCUUUUUGUUCAAAGUCUAUUUUUAUU | |
| | | CCUUGAUAUUUUUCUUUUUUUUUUUUUU | |
| | | UUUUUGUGGAUGGGGACUUGUGAAUUUU | |
| | | UCUAAAGGUGCUAUUUAACAUGGGAGGA | |
| | | GAGCGUGUGCGGCUCCAGCCCAGCCCGCU | |
| | | GCUCACUUUCCACCCUCUCUCCACCUGCC | |
| | | UCUGGCUUCUCAGGCCUCUGCUCUCCGAC | |
| | | CUCUCUCCUCUGAAACCCUCCUCCACAGC | |
| | | UGCAGCCCAUCCUCCCGGCUCCCUCCUAG | |
| | | UCUGUCCUGCGUCCUCUGUCCCCGGGUUU | |
| | | CAGAGACAACUUCCCAAAGCACAAAGCA | |
| | | GUUUUUCCCCCUAGGGGUGGGAGGAAGC | |
| | | AAAAGACUCUGUACCUAUUUUGUAUGUG | |
| | | UAUAAUAAUUUGAGAUGUUUUUAAUUAU | |
| | | UUUGAUUGCUGGAAUAAAGCAUGUGGAA | |
| | | AUGACCCAAACAUAAUCCGCAGUGGCCUC | |
| | | CUAAUUUCCUUCUUUGGAGUUGGGGGAG | |
| | | GGGUAGACAUGGGGAAGGGGCUUUGGGG | |
| | | UGAUGGGCUUGCCUUCCAUUCCUGCCCUU | |
| | | UCCCUCCCCACUAUUCUCUUCUAGAUCCC | |
| | | UCCAUAACCCCACUCCCCUUUCUCUCACC | |
| | | CUUCUUAUACCGCAAACCUUUCUACUUCC | |
| | | UCUUUCAUUUUCUAUUCUUGCAAUUUCC | |
| | | UUGCACCUUUUCCAAAUCCUCUUCUCCCC | |
| | | UGCAAUACCAUACAGGCAAUCCACGUGC | |
| | | ACAACACACACACACACUCUUCACAUCUG | |
| | | GGGUUGUCCAAACCUCAUACCCACUCCCC | |
| | | UUCAAGCCCAUCCACUCUCCACCCCCUGG | |
| | | AUGCCCUGCACUUGGUGGCGGUGGGAUG | |
| | | CUCAUGGAUACUGGGAGGGUGAGGGGAG | |
| | | UGGAACCCGUGAGGAGGACCUGGGGGCC | |
| | | UCUCCUUGAACUGACAUGAAGGGUCAUC | |
| | | UGGCCUCUGCUCCCUUCUCACCCACGCUG | |
| | | ACCUCCUGCCGAAGGAGCAACGCAACAGG | |
| | | AGAGGGGUCUGCUGAGCCUGGCGAGGGU | |
| | | CUGGGAGGGACCAGGAGGAAGGCGUGCU | |
| | | CCCUGCUCGCUGUCCUGGCCCUGGGGGAG | |
| | | UGAGGGAGACAGACACCUGGGAGAGCUG | |
| | | UGGGGAAGGCACUCGCACCGUGCUCUUG | |
| | | GGAAGGAAGGAGACCUGGCCCUGCUCAC | |
| | | CACGGACUGGGUGCCUCGACCUCCUGAAU | |
| | | CCCCAGAACACAACCCCCCUGGGCUGGGG | |
| | | UGGUCUGGGGAACCAUCGUGCCCCCGCCU | |
| | | CCCGCCUACUCCUUUUUAAGCUU | |
| 3′ UTR-015 | Plod1; | UUGGCCAGGCCUGACCCUCUUGGACCUUU | 231 |
| | procollagen- | CUUCUUUGCCGACAACCACUGCCCAGCAG | |
| | lysine, 2- | CCUCUGGGACCUCGGGGUCCCAGGGAACC | |
| | oxoglutarate | CAGUCCAGCCUCCUGGCUGUUGACUUCCC | |
| | 5- | AUUGCUCUUGGAGCCACCAAUCAAAGAG | |
| | dioxygenase 1 | AUUCAAAGAGAUUCCUGCAGGCCAGAGG | |
| | | CGGAACACACCUUUAUGGCUGGGGCUCU | |
| | | CCGUGGUGUUCUGGACCCAGCCCCUGGAG | |
| | | ACACCAUUCACUUUUACUGCUUUGUAGU | |
| | | GACUCGUGCUCUCCAACCUGUCUUCCUGA | |
| | | AAAACCAAGGCCCCCUUCCCCCACCUCUU | |
| | | CCAUGGGGUGAGACUUGAGCAGAACAGG | |
| | | GGCUUCCCCAAGUUGCCCAGAAAGACUG | |
| | | UCUGGGUGAGAAGCCAUGGCCAGAGCUU | |
| | | CUCCCAGGCACAGGUGUUGCACCAGGGAC | |
| | | UUCUGCUUCAAGUUUUGGGGUAAAGACA | |
| | | CCUGGAUCAGACUCCAAGGGCUGCCCUGA | |
| | | GUCUGGGACUUCUGCCUCCAUGGCUGGU | |
| | | CAUGAGAGCAAACCGUAGUCCCCUGGAG | |
| | | ACAGCGACUCCAGAGAACCUCUUGGGAG | |
| | | ACAGAAGAGGCAUCUGUGCACAGCUCGA | |
| | | UCUUCUACUUGCCUGUGGGGAGGGGAGU | |
| | | GACAGGUCCACACACCACACUGGGUCACC | |
| | | CUGUCCUGGAUGCCUCUGAAGAGAGGGA | |
| | | CAGACCGUCAGAAACUGGAGAGUUUCUA | |
| | | UUAAAGGUCAUUUAAACCA | |
| 3′ UTR-016 | Nucb1; | UCCUCCGGGACCCCAGCCCUCAGGAUUCC | 232 |
| | nucleobindin | UGAUGCUCCAAGGCGACUGAUGGGCGCU | |
| | 1 | GGAUGAAGUGGCACAGUCAGCUUCCCUG | |
| | | GGGGCUGGUGUCAUGUUGGGCUCCUGGG | |
| | | GCGGGGGCACGGCCUGGCAUUUCACGCA | |
| | | UUGCUGCCACCCCAGGUCCACCUGUCUCC | |
| | | ACUUUCACAGCCUCCAAGUCUGUGGCUCU | |
| | | UCCCUUCUGUCCUCCGAGGGGCUUGCCUU | |
| | | CUCUCGUGUCCAGUGAGGUGCUCAGUGA | |
| | | UCGGCUUAACUUAGAGAAGCCCGCCCCCU | |
| | | CCCCUUCUCCGUCUGUCCCAAGAGGGUCU | |
| | | GCUCUGAGCCUGCGUUCCUAGGUGGCUC | |
| | | GGCCUCAGCUGCCUGGGUUGUGGCCGCCC | |
| | | UAGCAUCCUGUAUGCCCACAGCUACUGG | |
| | | AAUCCCCGCUGCUGCUCCGGGCCAAGCUU | |
| | | CUGGUUGAUUAAUGAGGGCAUGGGGUGG | |
| | | UCCCUCAAGACCUUCCCCUACCUUUUGUG | |
| | | GAACCAGUGAUGCCUCAAAGACAGUGUC | |
| | | CCCUCCACAGCUGGGUGCCAGGGGCAGGG | |
| | | GAUCCUCAGUAUAGCCGGUGAACCCUGA | |
| | | UACCAGGAGCCUGGGCCUCCCUGAACCCC | |
| | | UGGCUUCCAGCCAUCUCAUCGCCAGCCUC | |
| | | CUCCUGGACCUCUUGGCCCCCAGCCCCUU | |
| | | CCCCACACAGCCCCAGAAGGGUCCCAGAG | |
| | | CUGACCCCACUCCAGGACCUAGGCCCAGC | |
| | | CCCUCAGCCUCAUCUGGAGCCCCUGAAGA | |
| | | CCAGUCCCACCCACCUUUCUGGCCUCAUC | |
| | | UGACACUGCUCCGCAUCCUGCUGUGUGUC | |
| | | CUGUUCCAUGUUCCGGUUCCAUCCAAAU | |
| | | ACACUUUCUGGAACAAA | |
| 3′ UTR-017 | α-globin-1 | GCUGGAGCCUCGGUGGCCAUGCUUCUUG | 233 |
| | | CCCCUUGGGCCUCCCCCCAGCCCCUCCUC | |
| | | CCCUUCCUGCACCCGUACCCCCGUGGUCU | |
| | | UUGAAUAAAGUCUGAGUGGGCGGC | |
| 3′UTR-018 | Downstream | UAAUAGGCUGGAGCCUCGGUGGCCAUGC | 234 |
| | UTR | UUCUUGCCCCUUGGGCCUCCCCCCAGCCC | |
| | | CUCCUCCCCUUCCUGCACCCGUACCCCCG | |
| | | UGGUCUUUGAAUAAAGUCUGAGUGGGCG | |
| | | GC | |
| 3′ UTR-019 | Downstream | UGAUAAUAGGCUGGAGCCUCGGUGGCCA | 235 |
| | UTR | UGCUUCUUGCCCCUUGGGCCUCCCCCCAG | |
| | | CCCCUCCUCCCCUUCCUGCACCCGUACCC | |
| | | CCUGGUCUUUGAAUAAAGUCUGAGUGGG | |
| | | CGGC | |
| 3′ UTR-020 | Downstream | UGAUAAUAGGCUGGAGCCUCGGUGGCCA | 236 |
| | UTR | UGCUUCUUGCCCCUUGGGCCUCCCCCCAG | |
| | | CCCCUCCUCCCCUUCCUGCACCCGUACCC | |
| | | CCGUGGUCUUUGAAUAAAGUCUGAGUGG | |
| | | GCGGC | |

In certain embodiments, the 3′ UTR sequence useful for the disclosure comprises a nucleotide sequence at least about 60%, at least about 70%, at least about 80%, at least about 90%, at least about 95%, at least about 96%, at least about 97%, at least about 98%, at least about 99%, or about 100% identical to a sequence selected from the group consisting of SEQ ID NO: 150, SEQ ID NO: 70 or any 3′UTR referred to by sequence in Table 10 and any combination thereof. In a particular embodiment, the 3′ UTR sequence further comprises a miRNA binding site, e.g., miR-122-3p binding site, a miR-122-5p binding site, a miR-143-3p binding site. In other embodiments, a 3′UTR sequence useful for the disclosure comprises 3′ UTR-018 (SEQ ID NO: 1027). In other embodiments, a 3′ UTR sequence useful for the disclosure comprises 3′ UTR comprised of nucleotide sequence set forth in SEQ ID NO: 150. In other embodiments, a 3′ UTR sequence useful for the disclosure comprises 3′ UTR comprised of nucleotide sequence set forth in SEQ ID NO: 70.

In certain embodiments, the 3′ UTR sequence comprises one or more miRNA binding sites, e.g., miR-122-3p binding site, a miR-122-5p binding site, a miR-143-3p binding site, or any other heterologous nucleotide sequences therein, without disrupting the function of the 3′ UTR. Some examples of 3′ UTR sequences comprising a miRNA binding site are listed in Table 11.

TABLE 11
Exemplary 3′ UTR with miRNA Binding Sites

| 3′ UTR | | | SEQ |
|---|---|---|---|
| Identifier/miRNA | Name/ | | ID |
| binding site | Description | Sequence | NO. |
| 3′ UTR-018 + miR- | Downstream | UAAUAGGCUGGAGCCUCGGUGGCCAUGC | 237 |
| 122-5p binding site | UTR | UUCUUGCCCCUUGGGCCUCCCCCCAGCCC | |
| | | CUCCUCCCCUUCCUGCACCCGUACCCCCC | |
| | | AAACACCAUUGUCACACUCCAGUGGUCU | |
| | | UUGAAUAAAGUCUGAGUGGGCGGC | |
| 3′ UTR-018 + miR- | Downstream | UAAUAGGCUGGAGCCUCGGUGGCCAUGC | 238 |
| 122-3p binding site | UTR | UUCUUGCCCCUUGGGCCUCCCCCCAGCCC | |
| | | CUCCUCCCCUUCCUGCACCCGUACCCCCU | |
| | | AUUUAGUGUGAUAAUGGCGUUGUGGUC | |
| | | UUUGAAUAAAGUCUGAGUGGGCGGC | |
| 3′ UTR-019 + miR- | Downstream | UGAUAAUAGGCUGGAGCCUCGGUGGCCA | 239 |
| 122-5p binding site | UTR | UGCUUCUUGCCCCUUGGGCCUCCCCCCAG | |
| | | CCCCUCCUCCCCUUCCUGCACCCGUACCC | |
| | | CCCAAACACCAUUGUCACACUCCAGUGG | |
| | | UCUUUGAAUAAAGUCUGAGUGGGCGGC | |
| 3′UTR + miR-142- | Downstream | GCUGGAGCCUCGGUGGCCAUGCUUCUUG | 240 |
| 3p binding site | UTR | CCCCUUGGGCCUCCCCCCAGCCCCUCCUC | |
| | | CCCUUCCUGCACCCGUACCCCCUCCAUAA | |
| | | AGUAGGAAACACUACAGUGGUCUUUGA | |
| | | AUAAAGUCUGAGUGGGCGGC | |

*miRNA binding site is bolded.

In certain embodiments, the 3′ UTR sequence useful for the disclosure comprises a nucleotide sequence at least about 60%, at least about 70%, at least about 80%, at least about t90%, at least about 95%, at least about 96%, at least about 97%, at least about 98%, at least about 99%, or about 100% identical to the sequence set forth as SEQ ID NO: 70 or SEQ ID NO: 235.

### Regions Having a 5′ Cap

The polynucleotide comprising an mRNA encoding a polypeptide of the present disclosure can further comprise a 5′ cap. The 5′ cap useful for polypeptide encoding mRNA can bind the mRNA Cap Binding Protein (CBP), thereby increasing mRNA stability. The cap can further assist the removal of 5′ proximal introns removal during mRNA splicing.

In some embodiments, the polynucleotide comprising an mRNA encoding a polypeptide of the present disclosure comprises a non-hydrolyzable cap structure preventing decapping and thus increasing mRNA half-life. Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, Mass.) can be used with α-thio-guanosine nucleotides according to the manufacturer's instructions to create a phosphorothioate linkage in the 5′-ppp-5′ cap. Additional modified guanosine nucleotides can be used such as α-methyl-phosphonate and seleno-phosphate nucleotides.

In certain embodiments, the 5′ cap comprises 2′-O-methylation of the ribose sugars of 5′-terminal and/or 5′-anteterminal nucleotides on the 2′-hydroxyl group of the sugar ring. In other embodiments, the caps for the polypeptide-encoding mRNA include cap analogs, which herein are also referred to as synthetic cap analogs, chemical caps, chemical cap analogs, or structural or functional cap analogs, differ from natural (i.e. endogenous, wild-type or physiological) 5′-caps in their chemical structure, while retaining cap function. Cap analogs can be chemically (i.e. non-enzymatically) or enzymatically synthesized and/or linked to the polynucleotides of the disclosure.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m7G-3′mppp-G; which can equivalently be designated 3′ O-Me-m7G(5′)ppp(5′)G). The 3′-O atom of the other, unmodified, guanine becomes linked to the 5′-terminal nucleotide of the capped polynucleotide. The N7- and 3′-O-methlyated guanine provides the terminal moiety of the capped polynucleotide.

Another exemplary cap is mCAP, which is similar to ARCA but has a 2′-O-methyl group on guanosine (i.e., N7,2′-O-dimethyl-guanosine-5′-triphosphate-5′-guanosine, m7Gm-ppp-G).

In some embodiments, the cap is a dinucleotide cap analog. As a non-limiting example, the dinucleotide cap analog can be modified at different phosphate positions with a boranophosphate group or a phophoroselenoate group such as the dinucleotide cap analogs described in U.S. Pat. No. 8,519,110.

In another embodiment, the cap is a cap analog is a N7-(4-chlorophenoxyethyl) substituted dinucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m3′-OG(5′)ppp(5′)G cap analog. See, e.g., the various cap analogs and the methods of synthesizing cap analogs described in Kore et al. (2013) Bioorganic & Medicinal Chemistry 21:4570-4574. In another embodiment, a cap analog of the present disclosure is a 4-chloro/bromophenoxyethyl analog.

While cap analogs allow for the concomitant capping of a polynucleotide or a region thereof, in an in vitro transcription reaction, up to 20% of transcripts can remain uncapped. This, as well as the structural differences of a cap analog from an endogenous 5′-cap structures of nucleic acids produced by the endogenous, cellular transcription machinery, can lead to reduced translational competency and reduced cellular stability.

An mRNA of the present disclosure can also be capped post-manufacture (whether IVT or chemical synthesis), using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects.

Non-limiting examples of more authentic 5′ cap structures of the present disclosure are those which, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the Capt structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N,pN2p (cap 0), 7mG(5′)ppp(5′)NlmpNp (cap 1), and 7mG(5′)-ppp(5′)NlmpN2mp (cap 2).

According to the present disclosure, 5′ terminal caps can include endogenous caps or cap analogs. According to the present disclosure, a 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

### 5′ Capping and a 5′Trinucleotide Cap

It is desirable to manufacture therapeutic RNAs enzymatically using in vitro transcription (IVT). In general, a DNA-dependent RNA polymerase transcribes a DNA template containing an appropriate promoter into an RNA transcript. The poly(A) tail can be generated co-transcriptionally by incorporating a poly(T) tract in the template DNA or separately by using a poly(A) polymerase. Eukaryotic mRNAs start with a 5′ cap (e.g., a 5′ m7GpppX cap). Typically, the 5′ cap begins with an inverted G with N⁷Me (required for eIF4E binding). A preferred cap, Cap1 contains 2′OMe at the +1 position) followed by any nucleoside at +2 position. This cap can be installed post-transcriptionally, e.g., enzymatically (after transcription) or co-transcriptionally (during transcription).

Post-transcriptional capping can be carried out using the vaccinia capping enzyme and allows for complete capping of the RNA, generating a cap 0 structure on RNA carrying a 5′ terminal triphosphate or diphosphate group, the cap 0 structure being required for efficient translation of the mRNA in vivo. The cap 0 structure can then be further modified into cap 1 using a cap-specific 2′O methyltransferase. Vaccinia capping enzyme and 2′O methyltransferase have been used to generate cap 0 and cap 1 structures on in vitro transcripts, for example, for use in transfecting eukaryotic cells or in mRNA therapeutic applications to drive protein synthesis. While post-transcriptional capping by vaccinia capping enzymes can yield either Cap 0 or Cap 1 structures, it is an expensive process when utilized for large-scale mRNA production, for example, vaccinia is costly and in limited supply and there can be difficulties in purifying an IVT mRNA (e.g., removing S-adenosylmethionine (SAM) and 2′O-methyltransferase). Moreover, capping can be incomplete due to inaccessibility of structured 5′ ends.

Co-transcriptional capping using a cap analog has certain advantages over vaccinia capping, for example, the process requires a simpler workflow (e.g., no need for a purification step between transcription and capping). Traditional co-transcriptional capping methods utilize the dinucleotide ARCA (anti-reverse cap analog) and yield Cap 0 structures. ARCA capping has drawbacks, however, for example, the resulting Cap 0 structures can be immunogenic and the process often results in low yields and/or poorly capped material. Another potential drawback of this approach is a theoretical capping efficiency of <100%, due to competition from the GTP for the starting nucleotide. For example, co-transcriptonal capping using ARCA typically requires a 10:1 ratio of ARCA:GTP to achieve >90% capping (needed to outcompete GTP for initiation).

In some embodiments, mRNAs of the disclosure are comprised of trinucleotide mRNA cap analogs, prepared using co-transcriptional capping methods (e.g., featuring T7 RNA polymerase) for the in vitro synthesis of mRNA. Use of a trinucleotide cap analog may provide a solution to several of the above-described problems associated with vaccinia or ARCA capping. In addition, the methods of co-transcriptional capping described provide flexibility in modifying the penultimate nucleobase which may alter binding behavior, or affect the affinity of these caps towards decapping enzymes, or both, thus potentially improving stability of the respective mRNA. An exemplary trinucleotide for use in the herein-described co-transcriptional capping methods is the m7GpppAG (GAG) trinucleotide. Use of this trinucleotide results in the nucleotide at the +1 position being A instead of G. Both+1G and +1A are caps that can be found in naturally-occurring mRNAs.

T7 RNA polymerase prefers to initiate with 5′ GTP. Accordingly, most conventional mRNA transcripts start with 5′-GGG (based on transcription from a T7 promoter sequence such as 5′TAATACGACTCACTATAGGGNNNNNNNNN . . . 3′ (TATA being referred to as the “TATA box”). T7 RNA polymerase typically transcribes DNA downstream of a T7 promoter (5′ TAATACGACTCACTA7TAG 3′, referencing the coding strand). T7 polymerase starts transcription at the underlined G in the promoter sequence. The polymerase then transcribes using the opposite strand as a template from 5->3′1 The first base in the transcript will be a G.

The herein-described processes capitalize on the fact that the T7 enzyme has limited initiation activity with the single nucleotide ATP, driving T7 to initiate with the trinucleotide rather than ATP. The process thus generates an mRNA product with >90% functional cap post-transcription. The process is an efficient “one-pot” mRNA production method that includes, for example, the GAG trinucleotide (GpppAG; ^{m}GpppAₘG) in equimolar concentration with the NTPs, GTP, ATP, CTP and UTP. The process features an “A-start” DNA template that initiates transcription with 5′ adenosine (A). As defined herein, “A-start” and “G-start” DNA templates are double-stranded DNA having requisite nucleosides in the template strand, such that the coding strand (and corresponding mRNA) begin with A or G, respectively. For example, a G-start DNA template features a template strand having the nucleobases CC complementary to GG immediately downstream of the TATA box in the T7 promoter (referencing the coding strand), and an A-start DNA template features a template strand having the nucleobases TC complementary to the AG immediately downstream of the TATA box in the T7 promoter (referencing the coding strand).

An exemplary T7 promoter sequence featured in an A-start DNA template of the present disclosure is depicted here:

5′TAATACGACTCACTATAAGNNNNNNNNNN...3′
3′ATTATGCTGAGTGATATTCNNNNNNNNNN...3′

The trinucleotide-based capping methods described herein provide flexibility in dictating the penultimate nucleobase. The trinucleotide capping methods of the present disclosure provide efficient production of capped mRNA, for example, 95-98% capped mRNA with a natural cap 1 structure.

### Trinucleotide Caps

Provided herein are co-transcriptional capping methods for ribonucleic acid (RNA) synthesis. That is, RNA is produced in a “one-pot” reaction, without the need for a separate capping reaction. Thus, the methods, in some embodiments, comprise reacting a DNA template with a T7 RNA polymerase variant, nucleoside triphosphates, and a cap analog under in vitro transcription reaction conditions to produce RNA transcript.

A cap analog may be, for example, a dinucleotide cap, a trinucleotide cap, or a tetranucleotide cap. In some embodiments, a cap analog is a dinucleotide cap. In some embodiments, a cap analog is a trinucleotide cap. In some embodiments, a cap analog is a tetranucleotide cap.

A trinucleotide cap, in some embodiments, comprises a compound of formula (I)

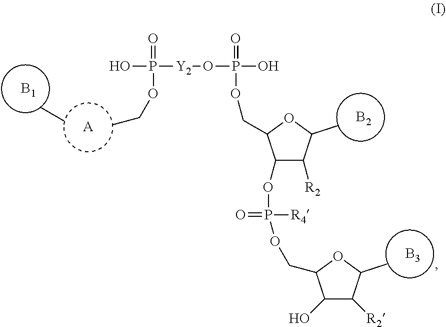

or a stereoisomer, tautomer or salt thereof, wherein

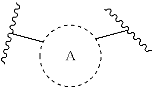

is

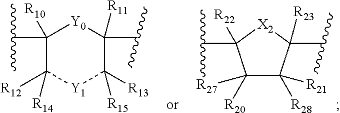

ring B₁ is a modified or unmodified Guanine;
ring B₂ and ring B₃ each independently is a nucleobase or a modified nucleobase;
X₂ is O, S(O)ₚ, NR₂₄, or CR₂₅R₂₆ in which p is 0, 1, or 2;
Y₀ is O or CR₆R₇;
Y1 is O, S(O)ₙ, CR₆R₇, or NR₈, in which n is 0, 1, or 2;
each — is a single bond or absent, wherein when each — is a single bond, Yi is O, S(O)ₙ, CR₆R₇, or NRs; and when each — is absent, Y₁ is void;
Y₂ is (OP(O)R₄)ₘ in which m is 0, 1, or 2, or —O—(CR₄₀R₄₁)u-Q₀-(CR₄₂R₄₃)v-, in which Q₀ is a bond, O, S(O)ᵣ, NR₄₄, or CR₄₅R₄₆, r is 0, 1, or 2, and each of u and v independently is 1, 2, 3 or 4;
each R₂ and R_{2′} independently is halo, LNA, or OR₃;
each R₃ independently is H, C1-C₆ alkyl, C₂-C₆ alkenyl, or C₂-C₆ alkynyl and R₃, when being C₁-C₆ alkyl, C₂-C₆ alkenyl, or C₂-C₆ alkynyl, is optionally substituted with one or more of halo, OH and C₁-C₆ alkoxyl that is optionally substituted with one or more OH or OC(O)—C₁-C₆ alkyl;
each R₄ and R_{4′} independently is H, halo, C₁-C₆ alkyl, OH, SH, SeH, or BH₃⁻;
each of R₆, R₇, and R₈, independently, is -Q₁-T₁, in which Q₁ is a bond or C₁-C₃ alkyl linker optionally substituted with one or more of halo, cyano, OH and C₁-C₆ alkoxy, and T₁ is H, halo, OH, COOH, cyano, or Rₛ₁, in which Rₛ₁ is C₁-C₃ alkyl, C₂-C₆ alkenyl, C₂-C₆ alkynyl, C₁-C₆ alkoxyl, C(O)O—C₁-C₆ alkyl, C₃-C₅ cycloalkyl, C₆-C₁₀ aryl, NR₃₁R₃₂, (NR₃₁R₃₂R₃₃)⁺, 4 to 12-membered heterocycloalkyl, or 5- or 6-membered heteroaryl, and Rₛ₁ is optionally substituted with one or more substituents selected from the group consisting of halo, OH, oxo, C₁-C₆ alkyl, COOH, C(O)O—C₁-C₆ alkyl, cyano, C₁-C₆ alkoxyl, NR₃₁R₃₂, (NR₃₁R₃₂R₃₃)+, C₃-C₅ cycloalkyl, C₆-C₁₀ aryl, 4 to 12-membered heterocycloalkyl, and 5- or 6-membered heteroaryl;
each of R₁₀, R₁₁, R₁₂, R₁₃R₁₄, and R₁₅, independently, is -Q₂-T₂, in which Q₂ is a bond or C₁-C₃ alkyl linker optionally substituted with one or more of halo, cyano, OH and C₁-C₆ alkoxy, and T₂ is H, halo, OH, NH₂, cyano, NO₂, N₃, Rₛ₂, or ORₛ₂, in which Rₛ₂ is C₁-C₆ alkyl, C₂-C₆ alkenyl, C₂-C₆ alkynyl, C₃-C₅ cycloalkyl, C₆-C₁₀ aryl, NHC(O)—C₁-C₆ alkyl, NR₃₁R₃₂, (NR₃₁R₃₂R₃₃)⁺, 4 to 12-membered heterocycloalkyl, or 5- or 6-membered heteroaryl, and Rₛ₂ is optionally substituted with one or more substituents selected from the group consisting of halo, OH, oxo, C₁-C₆ alkyl, COOH, C(O)O—C₁-C₆ alkyl, cyano, C₁-C₆ alkoxyl, NR₃₁R₃₂, (NR₃₁R₃₂R₃₃)⁺, C₃-C₅ cycloalkyl, C₆-C₁₀ aryl, 4 to 12-membered heterocycloalkyl, and 5- or 6-membered heteroaryl; or alternatively R₁₂ together with R₁₄ is oxo, or R₁₃ together with R₁₅ is oxo,
each of R₂₀, R₂₁, R₂₂, and R₂₃ independently is -Q₃-T₃, in which Q₃ is a bond or C₁-C₃ alkyl linker optionally substituted with one or more of halo, cyano, OH and C₁-C₆ alkoxy, and T₃ is H, halo, OH, NH₂, cyano, NO₂, N₃, Rₛ₃, or ORₛ₃, in which Rₛ₃ is C₁-C₆ alkyl, C₂-C₆ alkenyl, C₂-C₆ alkynyl, C₃-C₈ cycloalkyl, C₆-C₁₀ aryl, NHC(O)—C₁-C₆ alkyl, mono-C₁-C₆ alkylamino, di-C₁-C₆ alkylamino, 4 to 12-membered heterocycloalkyl, or 5- or 6-membered heteroaryl, and Rₛ₃ is optionally substituted with one or more substituents selected from the group consisting of halo, OH, oxo, C₁-C₆ alkyl, COOH, C(O)O—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl; each of R₂₄, R₂₅, and R₂₆ independently is H or C₁-C₆ alkyl;
each of R₂₇ and R₂₈ independently is H or OR₂₉; or R₂₇ and R₂₈ together form O—R₃₀—O; each R₂₉ independently is H, C₁-C₆ alkyl, C₂-C₆ alkenyl, or C₂-C₆ alkynyl and R₂₉, when being C₁-C₆ alkyl, C₂-C₆ alkenyl, or C₂-C₆ alkynyl, is optionally substituted with one or more of halo, OH and C₁-C₆ alkoxyl that is optionally substituted with one or more OH or OC(O)—C₁-C₆ alkyl;
R₃₀ is C₁-C₆ alkylene optionally substituted with one or more of halo, OH and C₁-C₆ alkoxyl;
each of R₃₁, R₃₂, and R₃₃, independently is H, C₁-C₆ alkyl, C₃-C₅ cycloalkyl, C₆-C₁₀ aryl, 4 to 12-membered heterocycloalkyl, or 5- or 6-membered heteroaryl;
each of R₄₀, R₄₁, R₄₂, and R₄₃ independently is H, halo, OH, cyano, N₃, OP(O)R₄₇R₄₈, or C₁-C₆ alkyl optionally substituted with one or more OP(O)R₄₇R₄₈, or one R₄₁ and one R₄₃, together with the carbon atoms to which they are attached and Q₀, form C₄-C₁₀ cycloalkyl, 4- to 14-membered heterocycloalkyl, C₆-C₁₀ aryl, or 5- to 14-membered heteroaryl, and each of the cycloalkyl, heterocycloalkyl, phenyl, or 5- to 6-membered heteroaryl is optionally substituted with one or more of OH, halo, cyano, N₃, oxo, OP(O)R₄₇R₄₈, C₁-C₆ alkyl, C₁-C₆ haloalkyl, COOH, C(O)O—C₁-C₆ alkyl, C₁-C₆ alkoxyl, C₁-C₆ haloalkoxyl, amino, mono-C₁-C₆ alkylamino, and di-C₁-C₆ alkylamino;
R₄₄ is H, C₁-C₆ alkyl, or an amine protecting group;
each of R₄₅ and R₄₆ independently is H, OP(O)R₄₇R₄₈, or C₁-C₆ alkyl optionally substituted with one or more OP(O)R₄₇R₄₈, and
each of R₄₇ and R₄₈, independently is H, halo, C₁-C₆ alkyl, OH, SH, SeH, or BH₃.

It should be understood that a cap analog, as provided herein, may include any of the cap analogs described in International Publication No. WO 2017/066797, published on 20 Apr. 2017, incorporated by reference herein in its entirety.

In some embodiments, the B₂ middle position can be a non-ribose molecule, such as arabinose.

In some embodiments R₂ is ethyl-based.

Thus, in some embodiments, a trinucleotide cap comprises the following structure:

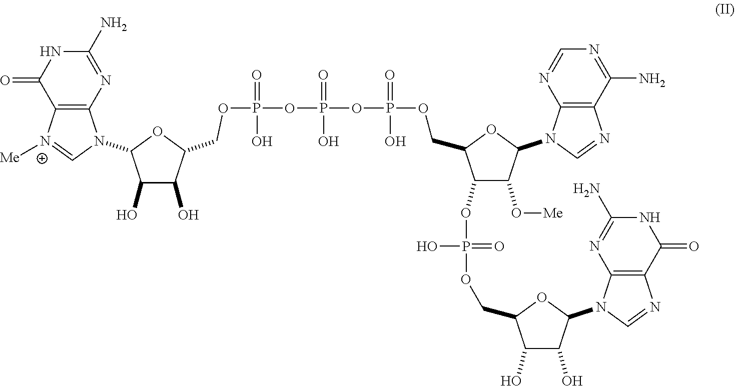

In other embodiments, a trinucleotide cap comprises the following structure:

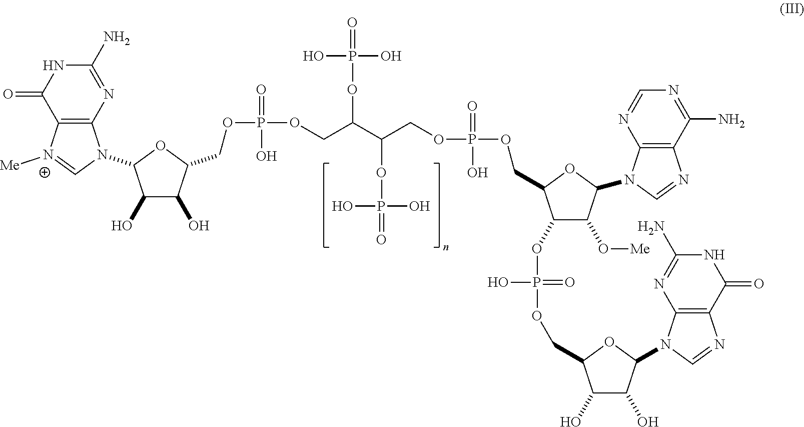

In yet other embodiments, a trinucleotide cap comprises the following structure:

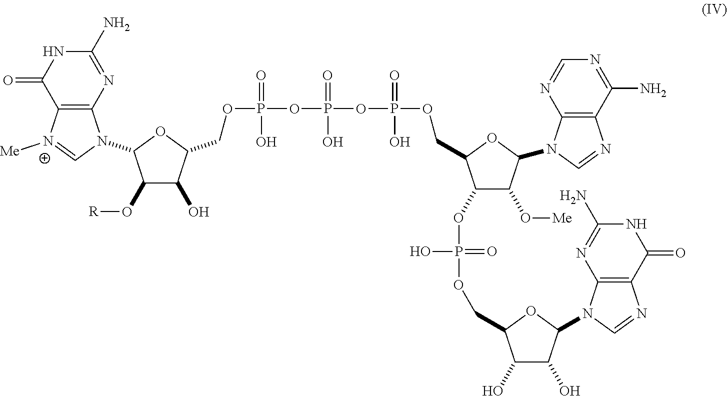

In still other embodiments, a trinucleotide cap comprises the following structure:

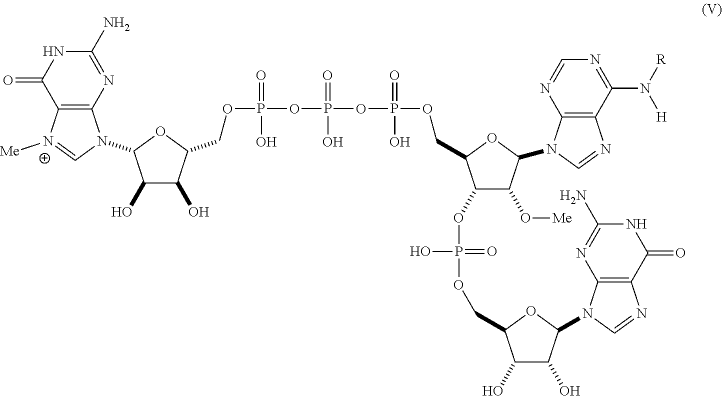

A trinucleotide cap, in some embodiments, comprises a sequence selected from the following sequences: GAA, GAC, GAG, GAU, GCA, GCC, GCG, GCU, GGA, GGC, GGG, GGU, GUA, GUC, GUG, and GUU.

In some embodiments, a trinucleotide cap comprises a sequence selected from the following sequences: m⁷GpppApA, m⁷GpppApC, m⁷GpppApG, m⁷GpppApU, m⁷GpppCpA, m⁷GpppCpC, m⁷GpppCpG, m⁷GpppCpU, m⁷GpppGpA, m⁷GpppGpC, m⁷GpppGpG, m⁷GpppGpU, m⁷GpppUpA, m⁷GpppUpC, m⁷GpppUpG, and m⁷GpppUpU.

A trinucleotide cap, in some embodiments, comprises a sequence selected from the following sequences: m⁷G_{3′OMe}pppApA, m⁷G_{3′OMe}pppApC, m⁷G_{3′OMe}pppApG, m⁷G_{3′OMe}pppApU, m⁷G_{3′OMe}pppCpA, m⁷G_{3′OMe}pppCpC, m⁷G_{3′OMe}pppCpG, m⁷G_{3′OMe}pppCpU, m⁷G_{3′OMe}pppGpA, m⁷G_{3′OMe}pppGpC, m⁷G_{3′OMe}pppGpG, m⁷G_{3′OMe}pppGpU, m⁷G_{3′OMe}pppUpA, m⁷G_{3′OMe}pppUpC, m⁷G_{3′OMe}pppUpG, and m⁷G_{3′OMe}pppUpU.

A trinucleotide cap, in other embodiments, comprises a sequence selected from the following sequences: m⁷G_{3′OMe}pppA_{2′OMe}pA, m⁷G_{3′OMe}pppA_{2′OMe}pC, m⁷G_{3′OMe}pppA_{2′OMe}pG, m⁷G_{3′OMe}ppp_{A2′OMe}pU, m⁷G_{3′OMe}pppC_{2′OMe}pA, m⁷G_{3′OMe}pppC_{2′OMe}pC, m⁷G_{3′OMe}pppC_{2′OMe}pG, m⁷G_{3′OMe}pppC_{2′OMe}pU, m⁷G_{3′OMe}pppG_{2′OMe}pA, m⁷G_{3′OMe}pppG_{2′OMe}pC, m⁷G_{3′OMe}pppG_{2′OMe}pG, m⁷G_{3′OMe}pppG_{2′OMe}pU, m⁷G_{3′OMe}pppU_{2′OMe}pA, m⁷G_{3′OMe}pppU_{2′OMe}pC, m⁷G_{3′OMe}pppU_{2′OMe}pG, and m⁷G_{3′OMe}pppU_{2′OMe}pU.

A trinucleotide cap, in still other embodiments, comprises a sequence selected from the following sequences: m⁷GpppA_{2′OMe}pA, m⁷GpppA_{2′OMe}pC, m⁷GpppA_{2′OMe}pG, m⁷GpppA_{2′OMe}pU, m⁷GpppC_{2′OMe}pA, m⁷GpppC_{2′OMe}pC, m⁷GpppC_{2′OMe}pG, m⁷GpppC_{2′OMe}pU, m⁷GpppG_{2′OMe}PA, m⁷GpppG_{2′OMe}pC, m⁷GpppG_{2′OMe}pG, m⁷GpppG_{2′OMe}pU, m⁷GpppU_{2′OMe}pA, m⁷GpppU_{2′OMe}PC, m⁷GpppU_{2′OMe}pG, and m⁷GpppU_{2′OMe}pU.

A trinucleotide cap, in further embodiments, comprises a sequence selected from the following sequences: m⁷Gpppm⁶A_{2′OMe}pA, m⁷Gpppm⁶A_{2′OMe}pC, and m⁷Gpppm⁶A_{2′OMe}pG, m⁷Gpppm⁶A_{2′OMe}pU

A trinucleotide cap, in yet other embodiments, comprises a sequence selected from the following sequences: m⁷Gpppe⁶A_{2′OMe}pA, m⁷Gpppe⁶A_{2′OMe}pC, and m⁷Gpppe⁶A_{2′OMe}pG,

In some embodiments, a trinucleotide cap comprises GAG. In some embodiments, a trinucleotide cap comprises GCG. In some embodiments, a trinucleotide cap comprises GUG. In some embodiments, a trinucleotide cap comprises GGG.

### Transcription

Some aspects of the present disclosure provide co-transcriptional capping methods that comprise reacting a DNA template with a RNA polymerase (e.g., T7 RNA polymerase), nucleoside triphosphates, and a trinucleotide cap analog under in vitro transcription reaction conditions to produce RNA transcript. A RNA transcript, in some embodiments, is a messenger RNA (mRNA) that includes a nucleotide sequence encoding a polypeptide (e.g., protein or peptide) of interest (e.g., biologics, antibodies, antigens (vaccines), and therapeutic proteins) linked to a polyA tail. In some embodiments, the mRNA is modified mRNA (mmRNA), which includes at least one modified nucleotide. In some embodiments, a modified mRNA is comprised of one or more RNA elements.

IVT conditions typically require a purified linear DNA template containing a promoter, nucleoside triphosphates, a buffer system that includes dithiothreitol (DTT) and magnesium ions, and a RNA polymerase. The exact conditions used in the transcription reaction depend on the amount of RNA needed for a specific application. Typical IVT reactions are performed by incubating a DNA template with a RNA polymerase and nucleoside triphosphates, including GTP, ATP, CTP, and UTP (or nucleotide analogs) in a transcription buffer. A RNA transcript having a 5′ terminal guanosine triphosphate is produced from this reaction.

A DNA template may encode a polypeptide of interest. A DNA template, in some embodiments, includes a RNA polymerase promoter (e.g., a T7 RNA polymerase promoter) located 5′ from and operably linked to a polynucleotide encoding a polypeptide of interest. A DNA template may also include a nucleotide sequence encoding a polyadenylation (polyA) tail located at the 3′ end of the gene of interest.

In some embodiments, the DNA template includes a 2′-deoxythymidine residue at template position+1. In some embodiments, the DNA template includes a 2′-deoxycytidine residue at template position+1. In some embodiments, the DNA template includes a 2′-deoxyadenosine residue at template position+1. In some embodiments, the DNA template includes a 2′-deoxyguanosine residue at template position+1.

In some embodiments, use of a DNA template that includes a 2′-deoxythymidine residue or 2′-deoxycytidine residue at template position+1 results in the production of RNA transcript, wherein greater than 80% (e.g., greater than 85%, greater than 90%, or greater than 95%) of the RNA transcript produced includes a functional cap. Thus, in some embodiments, a DNA template used, for example, in an IVT reaction, includes a 2′-deoxythymidine residue at template position+1. In other embodiments, a DNA template used, for example, in an IVT reaction, includes a 2′-deoxycytidine residue at template position+1.

The addition of nucleoside triphosphates (NTPs) to the 3′ end of a growing RNA strand is catalyzed by a RNA polymerase, such as T7 RNA polymerase. In some embodiments, the RNA polymerase is present in a reaction (e.g., an IVT reaction) at a concentration of 0.01 mg/ml to 1 mg/ml. For example, the RNA polymerase may be present in a reaction at a concentration of 0.01 mg/mL, 0.05 mg/ml, 0.1 mg/ml, 0.5 mg/ml or 1.0 mg/ml.

In some embodiments, a co-transcriptional capping method for RNA synthesis comprises reacting a DNA template with a RNA polymerase, nucleoside triphosphates, and a trinucleotide cap (e.g., comprising sequence GpppA_{2′Ome}pG), under in vitro transcription reaction conditions to produce RNA transcript, wherein the DNA template includes a 2′-deoxythymidine residue or a 2′-deoxycytidine residue at template position+1.

The combination of a RNA polymerase with a trinucleotide cap analog (e.g., GpppA_{2′Ome}pG), in an in vitro transcription reaction, for example, results in the production of RNA transcript, wherein greater than 80% of the RNA transcript produced includes a functional cap. In some embodiments, greater than 85% of the RNA transcript produced includes a functional cap. In some embodiments, greater than 90% of the RNA transcript produced includes a functional cap. In some embodiments, greater than 95% of the RNA transcript produced includes a functional cap. In some embodiments, greater than 96% of the RNA transcript produced includes a functional cap. In some embodiments, greater than 97% of the RNA transcript produced includes a functional cap. In some embodiments, greater than 98% of the RNA transcript produced includes a functional cap. In some embodiments, greater than 99% of the RNA transcript produced includes a functional cap.

In some embodiments, the disclosure provides an mRNA, wherein the 5′UTR is comprised of a 5′ trinucleotide cap and one or more RNA elements. In some embodiments, the 5′UTR comprises a 5′ trinucleotide cap and one or more structural RNA element comprising a stem-loop comprising a nucleotide sequence selected from the group consisting of: SEQ ID NO: 6 or SEQ ID NO: 47. In some embodiments, the 5′UTR comprises a 5′ trinucleotide cap and one or more structural RNA element comprising a stem-loop comprising a nucleotide sequence identified by SEQ ID NO: 6. In some embodiments, the 5′UTR comprising a 5′ trinucleotide cap and one or more structural RNA elements (e.g., RNAse P stem loop) comprises an “A-start” as described herein. In some embodiments, the 5′UTR comprises the nucleotide sequence of SEQ ID NO: 117. In some embodiments, the 5′UTR comprises the nucleotide sequence of SEQ ID NO: 121. In some embodiments, the 5′UTR comprises the nucleotide sequence of SEQ ID NO: 125.

### Poly-A Tails

In some embodiments, a polynucleotide comprising an mRNA encoding a polypeptide of the present disclosure further comprises a poly A tail. In further embodiments, terminal groups on the poly-A tail can be incorporated for stabilization. In other embodiments, a poly-A tail comprises des-3′ hydroxyl tails. The useful poly-A tails can also include structural moieties or 2′-Omethyl modifications as taught by Li et al. (2005) Current Biology 15:1501-1507.

In one embodiment, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides).

In some embodiments, the polynucleotide or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall polynucleotide or the length of a particular region of the polynucleotide. This design can be based on the length of a coding region, the length of a particular feature or region or based on the length of the ultimate product expressed from the polynucleotides.

In this context, the poly-A tail can be 10, 20, 30, 40, 50, 60, 70, 80, 90, or 100% greater in length than the polynucleotide or feature thereof. The poly-A tail can also be designed as a fraction of the polynucleotides to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein can enhance expression.

Additionally, multiple distinct polynucleotides can be linked together via the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In some embodiments, the polynucleotides of the present disclosure are designed to include a polyA-G Quartet region. The G-quartet is a cyclic hydrogen bonded array of four guanine nucleotides that can be formed by G-rich sequences in both DNA and RNA. In this embodiment, the G-quartet is incorporated at the end of the poly-A tail. The resultant polynucleotide is assayed for stability, protein production and other parameters including half-life at various time points. It has been discovered that the polyA-G quartet results in protein production from an mRNA equivalent to at least 75% of that seen using a poly-A tail of 120 nucleotides alone.

### Start Codon Region

In some embodiments, an mRNA of the present disclosure further comprises regions that are analogous to or function like a start codon region.

In some embodiments, the translation of a polynucleotide initiates on a codon which is not the start codon AUG. Translation of the polynucleotide can initiate on an alternative start codon such as, but not limited to, ACG, AGG, AAG, CTG/CUG, GTG/GUG, ATA/AUA, ATT/AUU, TTG/UUG. See Touriol et al. (2003) Biology of the Cell 95:169-178 and Matsuda and Mauro (2010) PLoS ONE 5:11. As a non-limiting example, the translation of a polynucleotide begins on the alternative start codon ACG. As another non-limiting example, polynucleotide translation begins on the alternative start codon CUG. As yet another non-limiting example, the translation of a polynucleotide begins on the alternative start codon GUG.

Nucleotides flanking a codon that initiates translation such as, but not limited to, a start codon or an alternative start codon, are known to affect the translation efficiency, the length and/or the structure of the polynucleotide. See, e.g., Matsuda and Mauro (2010) PLoS ONE 5:11. Masking any of the nucleotides flanking a codon that initiates translation can be used to alter the position of translation initiation, translation efficiency, length and/or structure of a polynucleotide.

In some embodiments, a masking agent is used near the start codon or alternative start codon in order to mask or hide the codon to reduce the probability of translation initiation at the masked start codon or alternative start codon. Non-limiting examples of masking agents include antisense locked nucleic acids (LNA) polynucleotides and exon-junction complexes (EJCs). See, e.g., Matsuda and Mauro (2010) PLoS ONE 5:11, describing masking agents LNA polynucleotides and EJCs.

In another embodiment, a masking agent is used to mask a start codon of a polynucleotide in order to increase the likelihood that translation will initiate on an alternative start codon. In some embodiments, a masking agent is used to mask a first start codon or alternative start codon in order to increase the chance that translation will initiate on a start codon or alternative start codon downstream to the masked start codon or alternative start codon.

In some embodiments, a start codon or alternative start codon is located within a perfect complement for a miR binding site. The perfect complement of a miR binding site can help control the translation, length and/or structure of the polynucleotide similar to a masking agent. As a non-limiting example, the start codon or alternative start codon is located in the middle of a perfect complement for a miR-122 binding site. The start codon or alternative start codon can be located after the first nucleotide, second nucleotide, third nucleotide, fourth nucleotide, fifth nucleotide, sixth nucleotide, seventh nucleotide, eighth nucleotide, ninth nucleotide, tenth nucleotide, eleventh nucleotide, twelfth nucleotide, thirteenth nucleotide, fourteenth nucleotide, fifteenth nucleotide, sixteenth nucleotide, seventeenth nucleotide, eighteenth nucleotide, nineteenth nucleotide, twentieth nucleotide or twenty-first nucleotide.

In another embodiment, the start codon of a polynucleotide is removed from the polynucleotide sequence in order to have the translation of the polynucleotide begin on a codon which is not the start codon. Translation of the polynucleotide can begin on the codon following the removed start codon or on a downstream start codon or an alternative start codon. In a non-limiting example, the start codon ATG or AUG is removed as the first 3 nucleotides of the polynucleotide sequence in order to have translation initiate on a downstream start codon or alternative start codon. The polynucleotide sequence where the start codon was removed can further comprise at least one masking agent for the downstream start codon and/or alternative start codons in order to control or attempt to control the initiation of translation, the length of the polynucleotide and/or the structure of the polynucleotide.

### Stop Codon Region

In some embodiments, an mRNA of the present disclosure comprises one or more stop codons to terminate translation. In some embodiments, an mRNA of the disclosure comprises one stop codon in the 3′UTR. In some embodiments, an mRNA of the disclosure comprises two stop codons in the 3′UTR. In some embodiments, an mRNA of the disclosure comprises three stop codons in the 3′UTR. In some embodiments, an mRNA of the disclosure comprises four stop codons in the 3′UTR. In some embodiments, an mRNA of the disclosure comprises five stop codons in the 3′UTR.

In some embodiments, an mRNA of the disclosure comprises one or more stop codons in the 3′UTR wherein the one or more stop codons are selected from a group consisting of: UGA, UAA, and UAG. In some embodiments, the one or more stop codons comprise the same sequence selected form a group consisting of: UGA, UAA, and UAG. In some embodiments, the one or more stop codons comprise different sequences selected from a group consisting of: UGA, UAA, and UAG.

In some embodiments, an mRNA of the present disclosure comprises a stop codon UGA and two additional stop codons, wherein the first and second additional stop codons are UGA, UAA, or UAG. In some embodiments, an mRNA of the disclosure comprises a stop codon UGA and two additional stop codons, wherein the first additional stop codon is UAA and the second additional stop codon is UAG.

### Adjusted Uracil Content

In some embodiments of the disclosure, an mRNA may have adjusted uracil content. In some embodiments, the uracil content of the open reading frame (ORF) of the polynucleotide encoding a therapeutic polypeptide relative to the theoretical minimum uracil content of a nucleotide sequence encoding the therapeutic polypeptide (% U_{TM}), is between about 100% and about 150. In some embodiments, the uracil content of the ORF is between about 105% and about 145%, about 105% and about 140%, about 110% and about 140%, about 110% and about 145%, about 115% and about 135%, about 105% and about 135%, about 110% and about 135%, about 115% and about 145%, or about 115% and about 140% of the theoretical minimum uracil content in the corresponding wild-type ORF (% U_{TM}). In other embodiments, the uracil content of the ORF is between about 117% and about 134% or between 118% and 132% of the % U_{TM}. In some embodiments, the uracil content of the ORF encoding a polypeptide is about 115%, about 120%, about 125%, about 130%, about 135%, about 140%, about 145%, or about 150% of the % U_{TM}. In this context, the term “uracil” can refer to an alternative uracil and/or naturally occurring uracil.

In some embodiments, the uracil content of the ORF of the polynucleotide relative to the uracil content of the corresponding wild-type ORF (% U_{WT}) is less than 100%. In some embodiments, the % U_{WT} of the polynucleotide is less than about 95%, less than about 90%, less than about 85%, less than 80%, less than 79%, less than 78%, less than 77%, less than 76%, less than 75%, less than 74%, or less than 73%. In some embodiments, the % U_{WT} of the polynucleotide is between 65% and 73%.

In some embodiments, the uracil content in the ORF of the mRNA encoding a is less than about 50%, about 40%, about 30%, or about 20% of the total nucleobase content in the ORF. In some embodiments, the uracil content in the ORF is between about 15% and about 25% of the total nucleobase content in the ORF. In other embodiments, the uracil content in the ORF is between about 20% and about 30% of the total nucleobase content in the ORF. In one embodiment, the uracil content in the ORF of the mRNA encoding a polypeptide is less than about 20% of the total nucleobase content in the open reading frame. In this context, the term “uracil” can refer to an alternative uracil and/or naturally occurring uracil.

In further embodiments, the ORF of the mRNA encoding a polypeptide having adjusted uracil content has increased cytosine (C), guanine (G), or guanine/cytosine (G/C) content (absolute or relative). In some embodiments, the overall increase in C, G, or G/C content (absolute or relative) of the ORF is at least about 2%, at least about 3%, at least about 4%, at least about 5%, at least about 6%, at least about 7%, at least about 10%, at least about 15%, at least about 20%, at least about 40%, at least about 50%, at least about 60%, at least about 70%, at least about 80%, at least about 90%, at least about 95%, or at least about 100% relative to the G/C content (absolute or relative) of the wild-type ORF. In some embodiments, the G, the C, or the G/C content in the ORF is less than about 100%, less than about 90%, less than about 85%, or less than about 80% of the theoretical maximum G, C, or G/C content of the nucleotide sequence encoding the PBDG polypeptide (% G_{TMX}; % C_{TMX}, or % G/C_{TMX}). In other embodiments, the G, the C, or the G/C content in the ORF is between about 70% and about 80%, between about 71% and about 79%, between about 71% and about 78%, or between about 71% and about 77% of the % G_{TMX}, % C_{TMX}, or % G/C_{TMX}. In some embodiments, the guanine content of the ORF of the polynucleotide with respect to the theoretical maximum guanine content of a nucleotide sequence encoding the polypeptide (% G_{TMX}) is at least 69%, at least 70%, at least 75%, at least about 80%, at least about 85%, at least about 90%, at least about 95%, or about 100%. In some embodiments, the % G_{TMX} of the polynucleotide is between about 70% and about 80%, between about 71% and about 79%, between about 71% and about 78%, or between about 71% and about 77%. In some embodiments, the cytosine content of the ORF of the polynucleotide relative to the theoretical maximum cytosine content of a nucleotide sequence encoding the polypeptide (% C_{TMX}) is at least 59%, at least 60%, at least about 65%, at least about 70%, at least about 75%, at least about 80%, at least about 85%, at least about 90%, at least about 95%, or about 100%. In some embodiments, the % C_{TMX} of the ORF of the polynucleotide is between about 60% and about 80%, between about 62% and about 80%, between about 63% and about 79%, or between about 68% and about 76%. In some embodiments, the guanine and cytosine content (G/C) of the ORF of the polynucleotide relative to the theoretical maximum G/C content in a nucleotide sequence encoding the polypeptide (% G/C_{TMX}) is at least about 81%, at least about 85%, at least about 90%, at least about 95%, or about 100%. In some embodiments, the % G/C_{TMX} in the ORF of the polynucleotide is between about 80% and about 100%, between about 85% and about 99%, between about 90% and about 97%, or between about 91% and about 96%. In some embodiments, the G/C content in the ORF of the polynucleotide relative to the G/C content in the corresponding wild-type ORF (% G/C_{WT}) is at least 102%, at least 103%, at least 104%, at least 105%, at least 106%, at least 107%, at least 110%, at least 115%, or at least 120%. In some embodiments, the average G/C content in the 3rd codon position in the ORF of the polynucleotide is at least 20%, at least 21%, at least 22%, at least 23%, at least 24%, at least 25%, at least 26%, at least 27%, at least 28%, at least 29%, or at least 30% higher than the average G/C content in the 3rd codon position in the corresponding wild-type ORF. In some embodiments, the increases in G and/or C content (absolute or relative) described herein can be conducted by replacing synonymous codons with low G, C, or G/C content with synonymous codons having higher G, C, or G/C content. In other embodiments, the increase in G and/or C content (absolute or relative) is conducted by replacing a codon ending with U with a synonymous codon ending with G or C.

In further embodiments, the ORF of the mRNA encoding a polypeptide includes less uracil pairs (UU) and/or uracil triplets (UUU) and/or uracil quadruplets (UUUU) than the corresponding wild-type nucleotide sequence encoding the polypeptide. In some embodiments, the ORF of the mRNA encoding a polypeptide of the disclosure includes no uracil pairs and/or uracil triplets and/or uracil quadruplets. In some embodiments, uracil pairs and/or uracil triplets and/or uracil quadruplets are reduced below a certain threshold, e.g., no more than 1, 2, 3, 4, 5, 6, 7, 8, 9, 10, 11, 12, 13, 14, 15, 16, 17, 18, 19, or 20 occurrences in the ORF of the mRNA encoding the polypeptide. In a particular embodiment, the ORF of the mRNA encoding the polypeptide of the disclosure contains less than 20, 19, 18, 17, 16, 15, 14, 13, 12, 11, 10, 9, 8, 7, 6, 5, 4, 3, 2, or 1 non-phenylalanine uracil pairs and/or triplets. In another embodiment, the ORF of the mRNA encoding the polypeptide contains no non-phenylalanine uracil pairs and/or triplets.

In further embodiments, the ORF of the mRNA encoding a polypeptide of the disclosure includes less uracil-rich clusters than the corresponding wild-type nucleotide sequence encoding the polypeptide. In some embodiments, the ORF of the mRNA encoding the polypeptide of the disclosure contains uracil-rich clusters that are shorter in length than corresponding uracil-rich clusters in the corresponding wild-type nucleotide sequence encoding the polypeptide.

In further embodiments, alternative lower frequency codons are employed. In some embodiment, the ORF of the polynucleotide further comprises at least one low-frequency codon. In some embodiments, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100% of the codons in the polypeptide-encoding ORF of the mRNA are substituted with alternative codons, each alternative codon having a codon frequency lower than the codon frequency of the substituted codon in the synonymous codon set. The ORF may also have adjusted uracil content, as described above. In some embodiments, at least one codon in the ORF of the mRNA encoding the polypeptide is substituted with an alternative codon having a codon frequency lower than the codon frequency of the substituted codon in the synonymous codon set.

In some embodiments, the polynucleotide is an mRNA that comprises an ORF that encodes a polypeptide, wherein the uracil content of the ORF is between about 115% and about 135% of the theoretical minimum uracil content in the corresponding wild-type ORF, and wherein the uracil content in the ORF encoding the polypeptide is less than about 30% of the total nucleobase content in the ORF. In some embodiments, the ORF that encodes the polypeptide is further modified to increase G/C content of the ORF (absolute or relative) by at least about 40%, as compared to the corresponding wild-type ORF. In yet other embodiments, the ORF encoding the polypeptide contains less than 20 non-phenylalanine uracil pairs and/or triplets. In some embodiments, at least one codon in the ORF of the mRNA encoding the polypeptide is further substituted with an alternative codon having a codon frequency lower than the codon frequency of the substituted codon in the synonymous codon set.

In some embodiments, the expression of the polypeptide encoded by an mRNA comprising an ORF, wherein the uracil content of the ORF has been adjusted (e.g., the uracil content is between about 115% and about 135% of the theoretical minimum uracil content in the corresponding wild-type ORF) is increased by at least about 10-fold when compared to expression of the polypeptide from the corresponding wild-type mRNA. In some embodiments, the innate immune response induced by the mRNA including an open ORF wherein the uracil content has been adjusted (e.g., the uracil content of the ORF is between about 115% and about 135% of the theoretical minimum uracil content in the corresponding wild-type ORF) is reduced by at least about 10-fold when compared to expression of the polypeptide from the corresponding wild-type mRNA. In some embodiments, the mRNA with adjusted uracil content does not substantially induce an innate immune response of a mammalian cell into which the mRNA is introduced.

In some embodiments, the uracil content of the mRNA is adjusted as described herein, and a modified nucleoside is partially or completely substituted for the uracil remaining in the mRNA following adjustment. As a non-limiting example, the natural nucleotide uridine may be substituted with a modified nucleoside as described herein. In some embodiments, the modified nucleoside comprises pseudouridine (ψ). In some embodiments, the modified nucleoside comprises 1-methyl-pseudouridine (mlw). In some embodiments, the modified nucleoside comprises 1-methyl-pseudouridine (mlw) and 5-methyl-cytidine (m5C). In some embodiments, the modified nucleoside comprises 2-thiouridine (s2U). In some embodiments, the modified nucleoside comprises 2-thiouridine and 5-methyl-cytidine (m5C). In some embodiments, the modified nucleoside comprises 5-methoxy-uridine (mo5U). In some embodiments, the modified nucleoside comprises 5-methoxy-uridine (mo5U) and 5-methyl-cytidine (m5C). In some embodiments, the modified nucleoside comprises 2′-O-methyl uridine. In some embodiments, the modified nucleoside comprises 2′-O-methyl uridine and 5-methyl-cytidine (m5C). In some embodiments, the modified nucleoside comprises N6-methyl-adenosine (m6A). In some embodiments, the modified nucleoside comprises N6-methyl-adenosine (m6A) and 5-methyl-cytidine (m5C).

### Removal of Endonuclease Sensitive Sequence Motifs

In some embodiments, an mRNA is altered for removal of sequences that are susceptible to degradation by endonuclease activity, referred to as endonuclease sensitive sequence motifs. In some embodiments, altering an mRNA to remove one or more endonuclease sensitive sequence motifs provides an mRNA with increased or improved stability, increased or improved half-life, and/or decreased susceptibility to endonuclease activity. In some embodiments, altering an mRNA to remove one or more endonuclease sensitive sequence motifs provides an mRNA with increased potency relative to an unaltered mRNA counterpart.

In some embodiments, an endonuclease sensitive sequence motif comprises the nucleotide sequence WGA, wherein W=adenine (A) or uracil (U). In some embodiments, a method of altering an mRNA to remove one or more endonuclease sensitive sequence motifs comprises altering the nucleotide sequence WGA, wherein W=adenine (A) or uracil (U), thereby increasing or improving stability, increasing or improving half-life, and/or decreasing susceptibility of the mRNA to endonucleases relative to an unaltered mRNA counterpart.

In some embodiments, the mRNA comprises at least one endonuclease sensitive sequence motif in a 5′UTR, ORF, and/or 3′UTR, wherein the endonuclease sensitive sequence motif is WGA, wherein W=adenine (A). In some embodiments, mRNA comprises at least one endonuclease sensitive sequence motif in a 5′UTR, ORF, and/or 3′UTR, wherein the endonuclease sensitive sequence motif is WGA, wherein W=uracil (U). In some embodiments, mRNA comprises at least one endonuclease sensitive sequence motif in a 5′UTR, ORF, and/or 3′UTR, wherein the endonuclease sensitive sequence motif is AGA or UGA. In some embodiments, the mRNA comprises at least one endonuclease sensitive sequence motif in a 5′UTR, ORF, and/or 3′UTR, wherein the endonuclease sensitive sequence motif is AGA. In some embodiments, the mRNA comprises at least one endonuclease sensitive sequence motif in a 5′UTR, ORF, and/or 3′UTR, wherein the endonuclease sensitive sequence motif is UGA.

In some embodiments, altering the at least one endonuclease sensitive sequence motif comprises introducing a substitution, insertion, deletion or chemical modification of at least one nucleotide comprising the endonuclease sensitive sequence motif. In some embodiments, altering the at least one endonuclease sensitive sequence motif comprises substitution of one or more nucleotides of the endonuclease sensitive motif with one or more different nucleotides, deleting one or more nucleotides from the endonuclease sensitive motif, replacing all of the nucleotides of the endonuclease sensitive motif with different nucleotides, deleting all of the nucleotides of the endonuclease sequence motif or a combination thereof (e.g., when there are two or more endonuclease sensitive sequence motifs in the mRNA) to alter, delete or replace one or more endonuclease sensitive motifs in the mRNA. In one embodiment, the at least one endonuclease sensitive sequence motif is altered by substitution of one or more nucleotides of the endonuclease sensitive sequence motif. In one embodiment, the at least one endonuclease sensitive sequence motif is altered by deletion of one or more nucleotides of the endonuclease sensitive sequence motif. In one embodiment, the at least one endonuclease sensitive sequence motif is altered by replacement of all of nucleotides of the endonuclease sensitive sequence motif with alternative nucleotides. In one embodiment, the at least one endonuclease sensitive sequence motif is altered by deletion of all of the nucleotides of the endonuclease sensitive sequence motif. In one embodiment, altering the at least one endonuclease sensitive sequence motif comprises chemically modifying at least one nucleotide of the endonuclease sensitive sequence motif.

In some embodiments, an mRNA of the disclosure comprises at least one endonuclease sensitive sequence motif in the 3′UTR comprising the nucleotide sequence UGA, wherein the nucleotide sequence UGA is positioned at the 5′end of the 3′UTR and is a stop codon in the first reading frame of the ORF, and wherein the nucleotide sequence UGA is substituted with a degenerate codon that is a stop codon (e.g., UAA, UAG). In some embodiments, an mRNA of the disclosure comprises at least one endonuclease sensitive sequence motif in the 3′UTR comprising the nucleotide sequence UGA, wherein the nucleotide sequence UGA is positioned at the 5′end of the 3′UTR and is a first stop codon in the first reading frame of the ORF, and wherein the nucleotide sequence UGA is altered by deletion.

In some embodiments, an mRNA of the disclosure comprises one or more stop codons in the 3′UTR wherein the one or more stop codons are selected from a group consisting of: UGA, UAA, and UAG. In some embodiments, the one or more stop codons comprise the same sequence selected form a group consisting of: UGA, UAA, and UAG. In some embodiments, the one or more stop codons comprise different sequences selected from a group consisting of: UGA, UAA, and UAG.

In some embodiments, an mRNA of the present disclosure comprises a stop codon UGA and two additional stop codons, wherein the first additional stop codon is UGA, UAA, or UAG and the second additional stop codon is UGA, UAA, or UAG. In some embodiments, an mRNA of the disclosure comprises a stop codon UGA and two additional stop codons, wherein the first additional stop codon is UAA and the second additional stop codon is UAG. In some embodiments, an mRNA of the disclosure comprising a stop codon UGA is altered by substitution or deletion of one or more nucleotides to increase or improve endonuclease resistance and/or decrease or reduce endonuclease susceptibility. In some embodiments, an mRNA of the disclosure comprising a stop codon UGA and one or more additional stop codons is altered by substitution or deletion to increase or improve endonuclease resistance and/or decrease or reduce endonuclease susceptibility. In some embodiments, the UGA stop codon is altered by substitution with a degenerate stop codon (e.g., UAA or UAG). In some embodiments, wherein the mRNA comprises a UGA stop codon and one or more additional stop codons, the UGA stop codon is altered by deletion. In some embodiments, altering a UGA stop codon of an mRNA of the disclosure increases or improves stability of the mRNA, increases or improves mRNA half-life, increases or improves mRNA potency, increases or improves endonuclease resistance, and/or decreases or reduces endonuclease susceptibility.

In some embodiments, an mRNA of the disclosure comprises three stop codons in the 3′UTR, wherein the first stop codon comprises the endonuclease senstivie sequence motif UGA and wherein the nucleotide sequence of the three stop codons is UGAUAAUAG as set forth by SEQ ID NO: 182. In some embodiments, an mRNA of the disclosure comprises three stop codons in the 3′UTR, wherein the first stop codon comprising the endonuclease senstivie sequence motif UGA is altered by deletion or substitution. In some embodiments, the first stop codon comprising the endonuclease senstivie sequence motif UGA is altered to UAA. In some embodiments, an mRNA of the disclosure comprises three stop codons in the 3′UTR, wherein the first stop codon comprising the endonuclease senstivie sequence motif UGA is altered to UAA, and wherein the nucleotide sequence of the three stop codons is UAAUAGUAA as set forth by SEQ ID NO: 183

### Chemical Modifications of RNA

Numerous approaches for the chemical modification of mRNA to improve translation efficiency and reduce immunogenicity are known, including modifications at the 5′ cap, 5′ and 3′-UTRs, the open reading frame, and the poly(A) tail (Sahin et al., (2014) Nat Rev Drug Discovery 13:759-780). For example, pseudouridine (ψ) modified mRNA was shown to increased expression of encoded erythropoietin (Kariko et al., (2012) Mol Ther 20:948-953). A combination of 2-thiouridine (s2U) and 5-methylcytidine (5meC) in modified mRNAs was shown to extend the expression of encoded protein (Kormann et al., (2011) Nat Biotechnol 29:154-157). A recent study demonstrated the induction of vascular regeneration using modified (5meC and ψ) mRNA encoding human vascular endothelial growth factor (Zangi et al., (2013) Nat Biotechnol 31:898-907). These studies demonstrate the utility of incorporating chemically modified nucleotides to achieve mRNA structural and functional optimization.

Accordingly, in some embodiments, an mRNA described herein comprises a modification, wherein the modification is the incorporation of one or more chemically modified nucleotides. In some embodiments, one or more chemically modified nucleotides is incorporated into the initiation codon of the mRNA and functions to increases binding affinity between the initiation codon and the anticodon of the initiator Met-tRNAiMet. In some embodiments, the one or more chemically modified nucleotides is 2-thiouridine. In some embodiments, the one or more chemically modified nucleotides is 2′-O-methyl-2-thiouridine. In some embodiments, the one or more chemically modified nucleotides is 2-selenouridine. In some embodiments, the one or more chemically modified nucleotides is 2′-O-methyl ribose. In some embodiments, the one or more chemically modified nucleotides is selected from a locked nucleic acid, inosine, 2-methylguanosine, or 6-methyl-adenosine. In some embodiments, deoxyribonucleotides are incorporated into mRNA. An mRNA of the disclosure may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified.

In some embodiments, an mRNA may instead or additionally include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine. In some embodiments, incorporation of a chain terminating nucleotide into an mRNA, for example at the 3′-terminus, may result in stabilization of the mRNA, as described, for example, in International Patent Publication No. WO 2013/103659.

An mRNA may instead or additionally include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. In some embodiments, a stem loop may affect one or more function(s) of an mRNA, such as initiation of translation, translation efficiency, and/or transcriptional termination.

An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. In some embodiments, a polyA sequence may affect the nuclear export, translation, and/or stability of an mRNA.

Modified mRNAs

In some embodiments, an mRNA of the disclosure comprises one or more modified nucleobases, nucleosides, or nucleotides (termed “modified mRNAs” or “mmRNAs”). In some embodiments, modified mRNAs may have useful properties, including enhanced stability, intracellular retention, enhanced translation, and/or the lack of a substantial induction of the innate immune response of a cell into which the mRNA is introduced, as compared to a reference unmodified mRNA. Therefore, use of modified mRNAs may enhance the efficiency of protein production, intracellular retention of nucleic acids, as well as possess reduced immunogenicity.

In some embodiments, an mRNA includes one or more (e.g., 1, 2, 3 or 4) different modified nucleobases, nucleosides, or nucleotides. In some embodiments, an mRNA includes one or more (e.g., 1, 2, 3, 4, 5, 6, 7, 8, 9, 10, 20, 30, 40, 50, 60, 70, 80, 90, 100, or more) different modified nucleobases, nucleosides, or nucleotides. In some embodiments, the modified mRNA may have reduced degradation in a cell into which the mRNA is introduced, relative to a corresponding unmodified mRNA.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridineor 5-bromo-uridine), 3-methyl-uridine (m³U), 5-methoxy-uridine (mo⁵U), uridine 5-oxyacetic acid (cmoSU), uridine 5-oxyacetic acid methyl ester (mcmoSU), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s2U), 5-aminomethyl-2-thio-uridine (nm⁵s2U), 5-methylaminomethyl-uridine (mnm⁵U), 5-methylaminomethyl-2-thio-uridine (mnm⁵s2U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s2U), 5-propynyl-uridine, 1-propynyl-pseudouridine, 5-taurinomethyl-uridine (τm⁵U), 1-taurinomethyl-pseudouridine, 5-taurinomethyl-2-thio-uridine(tm⁵s2U), 1-taurinomethyl-4-thio-pseudouridine, 5-methyl-uridine (m⁵U, i.e., having the nucleobase deoxythymine), 1-methyl-pseudouridine (m¹ψ), 5-methyl-2-thio-uridine (m⁵s2U), 1-methyl-4-thio-pseudouridine (m¹s⁴ψ), 4-thio-1-methyl-pseudouridine, 3-methyl-pseudouridine (m3j), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ψ), 5-(isopentenylaminomethyl)uridine (inmU), 5-(isopentenylaminomethyl)-2-thio-uridine (inm⁵s2U), α-thio-uridine, 2′-O-methyl-uridine (Urm), 5,2′-O-dimethyl-uridine (mTUm), 2′-O-methyl-pseudouridine (Wm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine. In some aspects, the modified uridine is N1-methyl-pseudouridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m³C), N4-acetyl-cytidine (ac⁴C), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k₂C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴²Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include α-thio-adenosine, 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenine (m²A), N6-methyl-adenosine (m⁶A), 2-methylthio-N6-methyl-adenosine (ms²m6A), N6-isopentenyl-adenosine (i⁶A), 2-methylthio-N6-isopentenyl-adenosine (ms²i6A), N6-(cis-hydroxyisopentenyl)adenosine (io⁶A), 2-methylthio-N6-(cis-hydroxyisopentenyl)adenosine (ms²io⁶A), N6-glycinylcarbamoyl-adenosine (g⁶A), N6-threonylcarbamoyl-adenosine (t⁶A), N6-methyl-N6-threonylcarbamoyl-adenosine (m⁶t6A), 2-methylthio-N6-threonylcarbamoyl-adenosine (ms²g6A), N6,N6-dimethyl-adenosine (m62A), N6-hydroxynorvalylcarbamoyl-adenosine (hn⁶A), 2-methylthio-N6-hydroxynorvalylcarbamoyl-adenosine (ms²hn⁶A), N6-acetyl-adenosine (ac⁶A), 7-methyl-adenine, 2-methylthio-adenine, 2-methoxy-adenine, α-thio-adenosine, 2′-O-methyl-adenosine (Am), N6,2′-O-dimethyl-adenosine (m⁶Am), N6,N6,2′-O-trimethyl-adenosine (m6₂Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include α-thio-guanosine, inosine (I), 1-methyl-inosine (m¹I), wyosine (imG), methylwyosine (mimG), 4-demethyl-wyosine (imG-14), isowyosine (imG2), wybutosine (yW), peroxywybutosine (o₂yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ₁), archaeosine (Ge), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (m¹G), N2-methyl-guanosine (m²G), N2,N2-dimethyl-guanosine (m22G), N2,7-dimethyl-guanosine (m²′7G), N2, N2,7-dimethyl-guanosine (m^{2,2,7}G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m22Gm), 1-methyl-2′-O-methyl-guanosine (m⁷Gm), N2,7-dimethyl-2′-O-methyl-guanosine (m^{2,7}Gm), 2′-O-methyl-inosine (Im), 1,2′-O-dimethyl-inosine (m¹Im), 2′-O-ribosylguanosine (phosphate) (Gr(p)), 1-thio-guanosine, 06-methyl-guanosine, 2′-F-ara-guanosine, and 2′-F-guanosine.

In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is pseudouridine (ψ), N1-methylpseudouridine (m¹ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified cytosine. Exemplary nucleobases and nucleosides having a modified cytosine include N4-acetyl-cytidine (ac⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, 2-thio-cytidine (s²C), 2-thio-5-methyl-cytidine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified adenine. Exemplary nucleobases and nucleosides having a modified adenine include 7-deaza-adenine, 1-methyl-adenosine (m¹A), 2-methyl-adenine (m²A), N6-methyl-adenosine (m⁶A). In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified guanine. Exemplary nucleobases and nucleosides having a modified guanine include inosine (I), 1-methyl-inosine (m¹I), wyosine (imG), methylwyosine (mimG), 7-deaza-guanosine, 7-cyano-7-deaza-guanosine (preQ₀), 7-aminomethyl-7-deaza-guanosine (preQ₁), 7-methyl-guanosine (m⁷G), 1-methyl-guanosine (m¹G), 8-oxo-guanosine, 7-methyl-8-oxo-guanosine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is 1-methyl-pseudouridine (m¹ψ), 5-methoxy-uridine (mo⁵U), 5-methyl-cytidine (m⁵C), pseudouridine (ψ), α-thio-guanosine, or α-thio-adenosine. In some embodiments, an mRNA of the disclosure includes a combination of one or more of the aforementioned modified nucleobases (e.g., a combination of 2, 3 or 4 of the aforementioned modified nucleobases.)

In some embodiments, the mRNA comprises pseudouridine (ψ). In some embodiments, the mRNA comprises pseudouridine (y) and 5-methyl-cytidine (m⁵C). In some embodiments, the mRNA comprises 1-methyl-pseudouridine (m¹ψ). In some embodiments, the mRNA comprises 1-methyl-pseudouridine (m¹ψ) and 5-methyl-cytidine (m⁵C). In some embodiments, the mRNA comprises 2-thiouridine (s²U). In some embodiments, the mRNA comprises 2-thiouridine and 5-methyl-cytidine (m⁵C). In some embodiments, the mRNA comprises 5-methoxy-uridine (mo⁵U). In some embodiments, the mRNA comprises 5-methoxy-uridine (mo⁵U) and 5-methyl-cytidine (m⁵C). In some embodiments, the mRNA comprises 2′-O-methyl uridine. In some embodiments, the mRNA comprises 2′-O-methyl uridine and 5-methyl-cytidine (m⁵C). In some embodiments, the mRNA comprises N6-methyl-adenosine (m⁶A). In some embodiments, the mRNA comprises N6-methyl-adenosine (m⁶A) and 5-methyl-cytidine (m⁵C).

In certain embodiments, an mRNA of the disclosure is uniformly modified (i.e., fully modified, modified through-out the entire sequence) for a particular modification. For example, an mRNA can be uniformly modified with 5-methyl-cytidine (m⁵C), meaning that all cytosine residues in the mRNA sequence are replaced with 5-methyl-cytidine (m⁵C). Similarly, mRNAs of the disclosure can be uniformly modified for any type of nucleoside residue present in the sequence by replacement with a modified residue such as those set forth above.

In some embodiments, an mRNA of the disclosure may be modified in a coding region (e.g., an open reading frame encoding a polypeptide). In other embodiments, an mRNA may be modified in regions besides a coding region. For example, in some embodiments, a 5′-UTR and/or a 3′-UTR are provided, wherein either or both may independently contain one or more different nucleoside modifications. In such embodiments, nucleoside modifications may also be present in the coding region.

Examples of nucleoside modifications and combinations thereof that may be present in mmRNAs of the present disclosure include, but are not limited to, those described in PCT Patent Application Publications: WO2012045075, WO2014081507, WO2014093924, WO2014164253, and WO2014159813.

The mmRNAs of the disclosure can include a combination of modifications to the sugar, the nucleobase, and/or the internucleoside linkage. These combinations can include any one or more modifications described herein.

Examples of modified nucleosides and modified nucleoside combinations are provided below in Table 12 and Table 13. These combinations of modified nucleotides can be used to form the mmRNAs of the disclosure. In certain embodiments, the modified nucleosides may be partially or completely substituted for the natural nucleotides of the mRNAs of the disclosure. As a non-limiting example, the natural nucleotide uridine may be substituted with a modified nucleoside described herein. In another non-limiting example, the natural nucleoside uridine may be partially substituted (e.g., about 0.1%, 1%, 5%, 10%, 15%, 20%, 25%, 30%, 35%, 40%, 45%, 50%, 55%, 60%, 65%, 70%, 75%, 80%, 85%, 90%, 95% or 99.9% of the natural uridines) with at least one of the modified nucleoside disclosed herein.

TABLE 12
Combinations of Nucleoside Modifications

| Modified Nucleotide | Modified Nucleotide Combination |
|---|---|
| α-thio-cytidine | α-thio-cytidine/5-iodo-uridine |
| | α-thio-cytidine/N1-methyl-pseudouridine |
| | α-thio-cytidine/α-thio-uridine |
| | α-thio-cytidine/5-methyl-uridine |
| | α-thio-cytidine/pseudo-uridine |
| | about 50% of the cytosines are α-thio-cytidine |
| pseudoisocytidine | pseudoisocytidine/5-iodo-uridine |
| | pseudoisocytidine/N1 -methyl-pseudouridine |
| | pseudoisocytidine/α-thio-uridine |
| | pseudoisocytidine/5-methyl-uridine |
| | pseudoisocytidine/pseudouridine |
| | about 25% of cytosines are pseudoisocytidine |
| | pseudoisocytidine/about 50% of uridines are N1-methyl-pseudouridine and |
| | about 50% of uridines are pseudouridine |
| | pseudoisocytidine/about 25% of uridines are N1-methyl-pseudouridine and |
| | about 25% of uridines are pseudouridine |
| pyrrolo-cytidine | pyrrolo-cytidine/5-iodo-uridine |
| | pyrrolo-cytidine/N1-methyl-pseudouridine |
| | pyrrolo-cytidine/α-thio-uridine |
| | pyrrolo-cytidine/5-methyl-uridine |
| | pyrrolo-cytidine/pseudouridine |
| | about 50% of the cytosines are pyrrolo-cytidine |
| 5-methyl-cytidine | 5-methyl-cytidine/5-iodo-uridine |
| | 5-methyl-cytidine/N1-methyl-pseudouridine |
| | 5-methyl-cytidine/α-thio-uridine |
| | 5-methyl-cytidine/5-methyl-uridine |
| | 5-methyl-cytidine/pseudouridine |
| | about 25% of cytosines are 5-methyl-cytidine |
| | about 50% of cytosines are 5-methyl-cytidine |
| | 5-methyl-cytidine/5-methoxy-uridine |
| | 5-methyl-cytidine/5-bromo-uridine |
| | 5-methyl-cytidine/2-thio-uridine |
| | 5-methyl-cytidine/about 50% of uridines are 2-thio-uridine |
| | about 50% of uridines are 5-methyl-cytidine/ |
| | about 50% of uridines are 2-thio-uridine |
| N4-acetyl-cytidine | N4-acetyl-cytidine/5-iodo-uridine |
| | N4-acetyl-cytidine/N1 -methyl-pseudouridine |
| | N4-acetyl-cytidine/a-thio-uridine |
| | N4-acetyl-cytidine/5-methyl-uridine |
| | N4-acetyl-cytidine/pseudouridine |
| | about 50% of cytosines are N4-acetyl-cytidine |
| | about 25% of cytosines are N4-acetyl-cytidine |
| | N4-acetyl-cytidine/5-methoxy-uridine |
| | N4-acetyl-cytidine/5-bromo-uridine |
| | N4-acetyl-cytidine/2-thio-uridine |
| | about 50% of cytosines are N4-acetyl-cytidine/ |
| | about 50% of uridines are 2-thio-uridine |

TABLE 13
Modified Nucleosides and Combinations Thereof

1-(2,2,2-Trifluoroethyl)pseudo-UTP
1-Ethyl-pseudo-UTP
1-Methyl-pseudo-U-alpha-thio-TP
1-methyl-pseudouridine TP, ATP, GTP, CTP
l-methyl-pseudo-UTP/5-methyl-CTP/ATP/GTP
1-methyl-pseudo-UTP/CTP/ATP/GTP
1-Propyl-pseudo-UTP
25% 5-Aminoallyl-CTP + 75% CTP/25% 5-Methoxy-UTP + 75% UTP
25% 5-Aminoallyl-CTP + 75% CTP/75% 5-Methoxy-UTP + 25% UTP
25% 5-Bromo-CTP + 75% CTP/25% 5-Methoxy-UTP + 75% UTP
25% 5-Bromo-CTP + 75% CTP/75% 5-Methoxy-UTP + 25% UTP
25% 5-Bromo-CTP + 75% CTP/1-Methyl-pseudo-UTP
25% 5-Carboxy-CTP + 75% CTP/25% 5-Methoxy-UTP + 75% UTP
25% 5-Carboxy-CTP + 75% CTP/75% 5-Methoxy-UTP + 25% UTP
25% 5-Ethyl-CTP + 75% CTP/25% 5-Methoxy-UTP + 75% UTP
25% 5-Ethyl-CTP + 75% CTP/75% 5-Methoxy-UTP + 25% UTP
25% 5-Ethynyl-CTP + 75% CTP/25% 5-Methoxy-UTP + 75% UTP
25% 5-Ethynyl-CTP + 75% CTP/75% 5-Methoxy-UTP + 25% UTP
25% 5-Fluoro-CTP + 75% CTP/ 25% 5-Methoxy-UTP + 75% UTP
25% 5-Fluoro-CTP + 75% CTP/75% 5-Methoxy-UTP + 25% UTP
25% 5-Formyl-CTP + 75% CTP/25% 5-Methoxy-UTP + 75% UTP
25% 5-Formyl-CTP + 75% CTP/75% 5-Methoxy-UTP + 25% UTP
25% 5-Hydroxymethyl-CTP + 75 % CTP/25% 5-Methoxy-UTP + 75% UTP
25% 5-Hydroxymethyl-CTP + 75% CTP/75% 5-Methoxy-UTP + 25% UTP
25% 5-Iodo-CTP + 75% CTP/25% 5-Methoxy-UTP + 75% UTP
25% 5-Iodo-CTP + 75% CTP/75% 5-Methoxy-UTP + 25% UTP
25% 5-Methoxy-CTP + 75% CTP/25% 5-Methoxy-UTP + 75% UTP
25% 5-Methoxy-CTP + 75% CTP/75% 5-Methoxy-UTP + 25% UTP
25% 5-Methyl-CTP + 75% CTP/25% 5-Methoxy-UTP + 75% 1-Methyl-pseudo-UTP
25% 5-Methyl-CTP + 75% CTP/25% 5-Methoxy-UTP + 75% UTP
25% 5-Methyl-CTP + 75% CTP/50% 5-Methoxy-UTP + 50% 1-Methyl-pseudo-UTP
25% 5-Methyl-CTP + 75% CTP/50% 5-Methoxy-UTP + 50% UTP
25% 5-Methyl-CTP + 75% CTP/5-Methoxy-UTP
25% 5-Methyl-CTP + 75% CTP/75% 5-Methoxy-UTP + 25% 1-Methyl-pseudo-UTP
25% 5-Methyl-CTP + 75% CTP/75% 5-Methoxy-UTP + 25% UTP
25% 5-Phenyl-CTP + 75% CTP/25% 5-Methoxy-UTP + 75% UTP
25% 5-Phenyl-CTP + 75% CTP/75% 5-Methoxy-UTP + 25% UTP
25% 5-Trifluoromethyl-CTP + 75% CTP/25% 5-Methoxy-UTP + 75% UTP
25% 5-Trifluoromethyl-CTP + 75% CTP/75% 5-Methoxy-UTP + 25% UTP
25% 5-Trifluoromethyl-CTP + 75% CTP/1-Methyl-pseudo-UTP
25% N4-Ac-CTP + 75% CTP/25% 5-Methoxy-UTP + 75% UTP
25% N4-Ac-CTP + 75% CTP/75% 5-Methoxy-UTP + 25% UTP
25% N4-Bz-CTP + 75% CTP/25% 5-Methoxy-UTP + 75% UTP
25% N4-Bz-CTP + 75% CTP/75% 5-Methoxy-UTP + 25% UTP
25% N4-Methyl-CTP + 75% CTP/25% 5-Methoxy-UTP + 75% UTP
25% N4-Methyl-CTP + 75% CTP/75% 5-Methoxy-UTP + 25% UTP
25% Pseudo-iso-CTP + 75% CTP/25% 5-Methoxy-UTP + 75% UTP
25% Pseudo-iso-CTP + 75% CTP/75% 5-Methoxy-UTP + 25% UTP
25% 5-Bromo-CTP/75% CTP/Pseudo-UTP
25% 5-methoxy-UTP/25% 5-methyl-CTP/ATP/GTP
25% 5-methoxy-UTP/5-methyl-CTP/ATP/GTP
25% 5-methoxy-UTP/75% 5-methyl-CTP/ATP/GTP
25% 5-methoxy-UTP/CTP/ATP/GTP
25% 5-methoxy-UTP/50% 5-methyl-CTP/ATP/GTP
2-Amino-ATP
2-Thio-CTP
2-thio-pseudouridine TP, ATP, GTP, CTP
2-Thio-pseudo-UTP
2-Thio-UTP
3-Methyl-CTP
3-Methyl-pseudo-UTP
4-Thio-UTP
50% 5-Bromo-CTP + 50% CTP/1-Methyl-pseudo-UTP
50% 5-Hydroxymethyl-CTP + 50% CTP/1-Methyl-pseudo-UTP
50% 5-methoxy-UTP/5-methyl-CTP/ATP/GTP
50% 5-Methyl-CTP + 50% CTP/25% 5-Methoxy-UTP + 75% 1-Methyl-pseudo-UTP
50% 5-Methyl-CTP + 50% CTP/25% 5-Methoxy-UTP + 75% UTP
50% 5-Methyl-CTP + 50% CTP/50% 5-Methoxy-UTP + 50% 1-Methyl-pseudo-UTP
50% 5-Methyl-CTP + 50% CTP/50% 5-Methoxy-UTP + 50% UTP
50% 5-Methyl-CTP + 50% CTP/5-Methoxy-UTP
50% 5-Methyl-CTP + 50% CTP/75% 5-Methoxy-UTP + 25% 1-Methyl-pseudo-UTP
50% 5-Methyl-CTP + 50% CTP/75 % 5-Methoxy-UTP + 25% UTP
50% 5-Trifluoromethyl-CTP + 50% CTP/1-Methyl-pseudo-UTP
50% 5-Bromo-CTP/ 50% CTP/Pseudo-UTP
50% 5-methoxy-UTP/25% 5-methyl-CTP/ATP/GTP
50% 5-methoxy-UTP/50% 5-methyl-CTP/ATP/GTP
50% 5-methoxy-UTP/75% 5-methyl-CTP/ATP/GTP
50% 5-methoxy-UTP/CTP/ATP/GTP
5-Aminoallyl-CTP
5-Aminoallyl-CTP/5-Methoxy-UTP
5-Aminoallyl-UTP
5-Bromo-CTP
5-Bromo-CTP/5-Methoxy-UTP
5-Bromo-CTP/1-Methyl-pseudo-UTP
5-Bromo-CTP/Pseudo-UTP
5-bromocytidine TP, ATP, GTP, UTP
5-Bromo-UTP
5-Carboxy-CTP/5-Methoxy-UTP
5-Ethyl-CTP/5-Methoxy-UTP
5-Ethynyl-CTP/5-Methoxy-UTP
5-Fluoro-CTP/5-Methoxy-UTP
5-Formyl-CTP/5-Methoxy-UTP
5-Hydroxy-methyl-CTP/5-Methoxy-UTP
5-Hydroxymethyl-CTP
5-Hydroxymethyl-CTP/1-Methyl-pseudo-UTP
5-Hydroxymethyl-CTP/5-Methoxy-UTP
5-hydroxymethyl-cytidine TP, ATP, GTP, UTP
5-Iodo-CTP/5-Methoxy-UTP
5-Me-CTP/5-Methoxy-UTP
5-Methoxy carbonyl methyl-UTP
5-Methoxy-CTP/5-Methoxy-UTP
5-methoxy-uridine TP, ATP, GTP, UTP
5-methoxy-UTP
5-Methoxy-UTP
5-Methoxy-UTP/N6-Isopentenyl-ATP
5-methoxy-UTP/25% 5-methyl-CTP/ATP/GTP
5-methoxy-UTP/5-methyl-CTP/ATP/GTP
5-methoxy-UTP/75% 5-methyl-CTP/ATP/GTP
5-methoxy-UTP/CTP/ATP/GTP
5-Methyl-2-thio-UTP
5-Methylaminomethyl-UTP
5-Methyl-CTP/5-Methoxy-UTP
5-Methyl-CTP/5-Methoxy-UTP(cap 0)
5-Methyl-CTP/5-Methoxy-UTP(No cap)
5-Methyl-CTP/25% 5-Methoxy-UTP + 75% 1-Methyl-pseudo-UTP
5-Methyl-CTP/25% 5-Methoxy-UTP + 75% UTP
5-Methyl-CTP/50% 5-Methoxy-UTP + 50% 1-Methyl-pseudo-UTP
5-Methyl-CTP/50% 5-Methoxy-UTP + 50% UTP
5-Methyl-CTP/5-Methoxy-UTP/N6-Me-ATP
5-Methyl-CTP/75% 5-Methoxy-UTP + 25% 1-Methyl-pseudo-UTP
5-Methyl-CTP/75% 5-Methoxy-UTP + 25% UTP
5-Phenyl-CTP/5-Methoxy-UTP
5-Trifluoro-methyl-CTP/5-Methoxy-UTP
5-Trifluoromethyl-CTP
5-Trifluoromethyl-CTP/5-Methoxy-UTP
5-Trifluoromethyl-CTP/1-Methyl-pseudo-UTP
5-Trifluoromethyl-CTP/Pseudo-UTP
5-Trifluoromethyl-UTP
5-trifluromethylcytidine TP, ATP, GTP, UTP
75% 5-Aminoallyl-CTP + 25% CTP/25% 5-Methoxy-UTP + 75% UTP
75% 5-Aminoallyl-CTP + 25% CTP/75% 5-Methoxy-UTP + 25% UTP
75% 5-Bromo-CTP + 25% CTP/25% 5-Methoxy-UTP + 75% UTP
75% 5-Bromo-CTP + 25% CTP/75% 5-Methoxy-UTP + 25% UTP
75% 5-Carboxy-CTP + 25% CTP/25% 5-Methoxy-UTP + 75% UTP
75% 5-Carboxy-CTP + 25% CTP/75% 5-Methoxy-UTP + 25% UTP
75% 5-Ethyl-CTP + 25% CTP/25% 5-Methoxy-UTP + 75% UTP
75% 5-Ethyl-CTP + 25% CTP/75% 5-Methoxy-UTP + 25% UTP
75% 5-Ethynyl-CTP + 25% CTP/25% 5-Methoxy-UTP + 75% UTP
75% 5-Ethynyl-CTP + 25% CTP/75% 5-Methoxy-UTP + 25% UTP
75% 5-Fluoro-CTP + 25% CTP/25% 5-Methoxy-UTP + 75% UTP
75% 5-Fluoro-CTP + 25% CTP/75% 5-Methoxy-UTP + 25% UTP
75% 5-Formyl-CTP + 25% CTP/25% 5-Methoxy-UTP + 75% UTP
75% 5-Formyl-CTP + 25% CTP/75% 5-Methoxy-UTP + 25% UTP
75% 5-Hydroxymethyl-CTP + 25% CTP/25% 5-Methoxy-UTP + 75% UTP
75% 5-Hydroxymethyl-CTP + 25% CTP/75% 5-Methoxy-UTP + 25% UTP
75% 5-Iodo-CTP + 25% CTP/25% 5-Methoxy-UTP + 75% UTP
75% 5-Iodo-CTP + 25% CTP/75% 5-Methoxy-UTP + 25% UTP
75% 5-Methoxy-CTP + 25% CTP/25% 5-Methoxy-UTP + 75% UTP
75% 5-Methoxy-CTP + 25% CTP/75% 5-Methoxy-UTP + 25% UTP
75% 5-methoxy-UTP/5-methyl-CTP/ATP/GTP
75% 5-Methyl-CTP + 25% CTP/25% 5-Methoxy-UTP + 75% 1-Methyl-pseudo-UTP
75% 5-Methyl-CTP + 25% CTP/25% 5-Methoxy-UTP + 75% UTP
75% 5-Methyl-CTP + 25% CTP/50% 5-Methoxy-UTP + 50% 1-Methyl-pseudo-UTP
75% 5-Methyl-CTP + 25% CTP/50% 5-Methoxy-UTP + 50% UTP
75% 5-Methyl-CTP + 25% CTP/5-Methoxy-UTP
75% 5-Methyl-CTP + 25% CTP/75% 5-Methoxy-UTP + 25% 1-Methyl-pseudo-UTP
75% 5-Methyl-CTP + 25% CTP/75% 5-Methoxy-UTP + 25% UTP
75% 5-Phenyl-CTP + 25% CTP/25% 5-Methoxy-UTP + 75% UTP
75% 5-Phenyl-CTP + 25% CTP/75% 5-Methoxy-UTP + 25% UTP
75% 5-Trifluoromethyl-CTP + 25% CTP/25% 5-Methoxy-UTP + 75% UTP
75% 5-Trifluoromethyl-CTP + 25% CTP/75% 5-Methoxy-UTP + 25% UTP
75% 5-Trifluoromethyl-CTP + 25% CTP/1-Methyl-pseudo-UTP
75% N4-Ac-CTP + 25% CTP/25% 5-Methoxy-UTP + 75% UTP
75% N4-Ac-CTP + 25% CTP/75% 5-Methoxy-UTP + 25% UTP
75% N4-Bz-CTP + 25% CTP/25% 5-Methoxy-UTP + 75% UTP
75% N4-Bz-CTP + 25% CTP/75% 5-Methoxy-UTP + 25% UTP
75% N4-Methyl-CTP + 25% CTP/25% 5-Methoxy-UTP + 75% UTP
75% N4-Methyl-CTP + 25% CTP/75% 5-Methoxy-UTP + 25% UTP
75% Pseudo-iso-CTP + 25% CTP/25% 5-Methoxy-UTP + 75% UTP
75% Pseudo-iso-CTP + 25% CTP/75% 5-Methoxy-UTP + 25% UTP
75% 5-Bromo-CTP/25% CTP/1-Methyl-pseudo-UTP
75% 5-Bromo-CTP/25% CTP/Pseudo-UTP
75% 5-methoxy-UTP/25% 5-methyl-CTP/ATP/GTP
75% 5-methoxy-UTP/50% 5-methyl-CTP/ATP/GTP
75% 5-methoxy-UTP/75% 5-methyl-CTP/ATP/GTP
75% 5-methoxy-UTP/CTP/ATP/GTP
8-Aza-ATP
Alpha-thio-CTP
CTP/25% 5-Methoxy-UTP + 75% 1-Methyl-pseudo-UTP
CTP/25% 5-Methoxy-UTP + 75% UTP
CTP/50% 5-Methoxy-UTP + 50% 1-Methyl-pseudo-UTP
CTP/50% 5-Methoxy-UTP + 50% UTP
CTP/5-Methoxy-UTP
CTP/5-Methoxy-UTP (cap 0)
CTP/5-Methoxy-UTP(No cap)
CTP/75% 5-Methoxy-UTP + 25% 1-Methyl-pseudo-UTP
CTP/75% 5-Methoxy-UTP + 25% UTP
CTP/UTP(No cap)
N1-Me-GTP
N4-Ac-CTP
N4Ac-CTP/1-Methyl-pseudo-UTP
N4Ac-CTP/5-Methoxy-UTP
N4-acetyl-cytidine TP, ATP, GTP, UTP
N4-Bz-CTP/5-Methoxy-UTP
N4-methyl CTP
N4-Methyl-CTP/5-Methoxy-UTP
Pseudo-iso-CTP/5-Methoxy-UTP
PseudoU- alpha-thio-TP
pseudouridine TP, ATP, GTP, CTP
pseudo-UTP/5-methyl-CTP/ATP/GTP
UTP-5-oxyacetic acid Me ester
Xanthosine

According to the disclosure, polynucleotides of the disclosure may be synthesized to comprise the combinations or single modifications of Table 12 or Table 13.

Where a single modification is listed, the listed nucleoside or nucleotide represents 100 percent of that A, U, G or C nucleotide or nucleoside having been modified. Where percentages are listed, these represent the percentage of that particular A, U, G or C nucleobase triphosphate of the total amount of A, U, G, or C triphosphate present. For example, the combination: 25% 5-Aminoallyl-CTP+75% CTP/25% 5-Methoxy-UTP+75% UTP refers to a polynucleotide where 25% of the cytosine triphosphates are 5-Aminoallyl-CTP while 75% of the cytosines are CTP; whereas 25% of the uracils are 5-methoxy UTP while 75% of the uracils are UTP. Where no modified UTP is listed then the naturally occurring ATP, UTP, GTP and/or CTP is used at 100% of the sites of those nucleotides found in the polynucleotide. In this example all of the GTP and ATP nucleotides are left unmodified.

In certain embodiments, the present disclosure includes polynucleotides having at least 80%, at least 85%, at least 90%, at least 95%, at least 98%, or at least 99% sequence identity to any of the polynucleotide sequences described herein.

mRNAs of the present disclosure may be produced by means available in the art, including but not limited to in vitro transcription (IVT) and synthetic methods. Enzymatic (IVT), solid-phase, liquid-phase, combined synthetic methods, small region synthesis, and ligation methods may be utilized. In one embodiment, mRNAs are made using IVT enzymatic synthesis methods. Methods of making polynucleotides by IVT are known in the art and are described in International Application PCT/US2013/30062, the contents of which are incorporated herein by reference in their entirety. Accordingly, the present disclosure also includes polynucleotides, e.g., DNA, constructs and vectors that may be used to in vitro transcribe an mRNA described herein.

Non-natural modified nucleobases may be introduced into polynucleotides, e.g., mRNA, during synthesis or post-synthesis. In certain embodiments, modifications may be on internucleoside linkages, purine or pyrimidine bases, or sugar. In particular embodiments, the modification may be introduced at the terminal of a polynucleotide chain or anywhere else in the polynucleotide chain; with chemical synthesis or with a polymerase enzyme. Examples of modified nucleic acids and their synthesis are disclosed in PCT application No. PCT/US2012/058519. Synthesis of modified polynucleotides is also described in Verma and Eckstein, Annual Review of Biochemistry, vol. 76, 99-134 (1998).

Either enzymatic or chemical ligation methods may be used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

MicroRNA (miRNA) Binding Sites

Nucleic acid molecules (e.g., RNA, e.g., mRNA) of the disclosure can include regulatory elements, for example, microRNA (miRNA) binding sites, transcription factor binding sites, structured mRNA sequences and/or motifs, artificial binding sites engineered to act as pseudo-receptors for endogenous nucleic acid binding molecules, and combinations thereof. In some embodiments, nucleic acid molecules (e.g., RNA, e.g., mRNA) including such regulatory elements are referred to as including “sensor sequences.” Non-limiting examples of sensor sequences are described in U.S. Publication 2014/0200261, the contents of which are incorporated herein by reference in their entirety.

In some embodiments, a nucleic acid molecule (e.g., RNA, e.g., mRNA) of the disclosure comprises an open reading frame (ORF) encoding a polypeptide of interest and further comprises one or more miRNA binding site(s). Inclusion or incorporation of miRNA binding site(s) provides for regulation of nucleic acid molecules (e.g., RNA, e.g., mRNA) of the disclosure, and in turn, of the polypeptides encoded therefrom, based on tissue-specific and/or cell-type specific expression of naturally-occurring miRNAs.

A miRNA, e.g., a natural-occurring miRNA, is a 19-25 nucleotide long noncoding RNA that binds to a nucleic acid molecule (e.g., RNA, e.g., mRNA) and down-regulates gene expression either by reducing stability or by inhibiting translation of the polynucleotide. A miRNA sequence comprises a “seed” region, i.e., a sequence in the region of positions 2-8 of the mature miRNA. A miRNA seed can comprise positions 2-8 or 2-7 of the mature miRNA. In some embodiments, a miRNA seed can comprise 7 nucleotides (e.g., nucleotides 2-8 of the mature miRNA), wherein the seed-complementary site in the corresponding miRNA binding site is flanked by an adenosine (A) opposed to miRNA position 1. In some embodiments, a miRNA seed can comprise 6 nucleotides (e.g., nucleotides 2-7 of the mature miRNA), wherein the seed-complementary site in the corresponding miRNA binding site is flanked by an adenosine (A) opposed to miRNA position 1. See, for example, Grimson A, Farh K K, Johnston W K, Garrett-Engele P, Lim L P, Bartel D P; Mol Cell. 2007 Jul. 6; 27(1):91-105, miRNA profiling of the target cells or tissues can be conducted to determine the presence or absence of miRNA in the cells or tissues. In some embodiments, a nucleic acid molecule (e.g., RNA, e.g., mRNA) of the disclosure comprises one or more microRNA binding sites, microRNA target sequences, microRNA complementary sequences, or microRNA seed complementary sequences. Such sequences can correspond to, e.g., have complementarity to, any known microRNA such as those taught in US Publication US2005/0261218 and US Publication US2005/0059005, the contents of each of which are incorporated herein by reference in their entirety.

As used herein, the term “microRNA (miRNA or miR) binding site” refers to a sequence within a nucleic acid molecule, e.g., within a DNA or within an RNA transcript, including in the 5′UTR and/or 3′UTR, that has sufficient complementarity to all or a region of a miRNA to interact with, associate with or bind to the miRNA. In some embodiments, a nucleic acid molecule (e.g., RNA, e.g., mRNA) of the disclosure comprising an ORF encoding a polypeptide of interest and further comprises one or more miRNA binding site(s). In exemplary embodiments, a 5′UTR and/or 3′UTR of the nucleic acid molecule (e.g., RNA, e.g., mRNA) comprises the one or more miRNA binding site(s).

A miRNA binding site having sufficient complementarity to a miRNA refers to a degree of complementarity sufficient to facilitate miRNA-mediated regulation of a nucleic acid molecule (e.g., RNA, e.g., mRNA), e.g., miRNA-mediated translational repression or degradation of the nucleic acid molecule (e.g., RNA, e.g., mRNA). In exemplary aspects of the disclosure, a miRNA binding site having sufficient complementarity to the miRNA refers to a degree of complementarity sufficient to facilitate miRNA-mediated degradation of the nucleic acid molecule (e.g., RNA, e.g., mRNA), e.g., miRNA-guided RNA-induced silencing complex (RISC)-mediated cleavage of mRNA. The miRNA binding site can have complementarity to, for example, a 19-25 nucleotide miRNA sequence, to a 19-23 nucleotide miRNA sequence, or to a 22 nucleotide miRNA sequence. A miRNA binding site can be complementary to only a portion of a miRNA, e.g., to a portion less than 1, 2, 3, or 4 nucleotides of the full length of a naturally-occurring miRNA sequence. Full or complete complementarity (e.g., full complementarity or complete complementarity over all or a significant portion of the length of a naturally-occurring miRNA) is preferred when the desired regulation is mRNA degradation.

In some embodiments, a miRNA binding site includes a sequence that has complementarity (e.g., partial or complete complementarity) with a miRNA seed sequence. In some embodiments, the miRNA binding site includes a sequence that has complete complementarity with a miRNA seed sequence. In some embodiments, a miRNA binding site includes a sequence that has complementarity (e.g., partial or complete complementarity) with an miRNA sequence. In some embodiments, the miRNA binding site includes a sequence that has complete complementarity with a miRNA sequence. In some embodiments, a miRNA binding site has complete complementarity with a miRNA sequence but for 1, 2, or 3 nucleotide substitutions, terminal additions, and/or truncations.

In some embodiments, the miRNA binding site is the same length as the corresponding miRNA. In other embodiments, the miRNA binding site is one, two, three, four, five, six, seven, eight, nine, ten, eleven or twelve nucleotide(s) shorter than the corresponding miRNA at the 5′ terminus, the 3′ terminus, or both. In still other embodiments, the microRNA binding site is two nucleotides shorter than the corresponding microRNA at the 5′ terminus, the 3′ terminus, or both. The miRNA binding sites that are shorter than the corresponding miRNAs are still capable of degrading the mRNA incorporating one or more of the miRNA binding sites or preventing the mRNA from translation.

In some embodiments, the miRNA binding site binds the corresponding mature miRNA that is part of an active RISC containing Dicer. In another embodiment, binding of the miRNA binding site to the corresponding miRNA in RISC degrades the mRNA containing the miRNA binding site or prevents the mRNA from being translated. In some embodiments, the miRNA binding site has sufficient complementarity to miRNA so that a RISC complex comprising the miRNA cleaves the nucleic acid molecule (e.g., RNA, e.g., mRNA) comprising the miRNA binding site. In other embodiments, the miRNA binding site has imperfect complementarity so that a RISC complex comprising the miRNA induces instability in the nucleic acid molecule (e.g., RNA, e.g., mRNA) comprising the miRNA binding site. In another embodiment, the miRNA binding site has imperfect complementarity so that a RISC complex comprising the miRNA represses transcription of the nucleic acid molecule (e.g., RNA, e.g., mRNA) comprising the miRNA binding site.

In some embodiments, the miRNA binding site has one, two, three, four, five, six, seven, eight, nine, ten, eleven or twelve mismatch(es) from the corresponding miRNA.

In some embodiments, the miRNA binding site has at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one contiguous nucleotides complementary to at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one, respectively, contiguous nucleotides of the corresponding miRNA.

By engineering one or more miRNA binding sites into a nucleic acid molecule (e.g., RNA, e.g., mRNA) of the disclosure, the nucleic acid molecule (e.g., RNA, e.g., mRNA) can be targeted for degradation or reduced translation, provided the miRNA in question is available. This can reduce off-target effects upon delivery of the nucleic acid molecule (e.g., RNA, e.g., mRNA). For example, if a nucleic acid molecule (e.g., RNA, e.g., mRNA) of the disclosure is not intended to be delivered to a tissue or cell but ends up is said tissue or cell, then a miRNA abundant in the tissue or cell can inhibit the expression of the gene of interest if one or multiple binding sites of the miRNA are engineered into the 5′UTR and/or 3′UTR of the nucleic acid molecule (e.g., RNA, e.g., mRNA).

For example, one of skill in the art would understand that one or more miR can be included in a nucleic acid molecule (e.g., an RNA, e.g., mRNA) to minimize expression in cell types other than lymphoid cells. In one embodiment, miR122 can be used. In another embodiment, miR126 can be used. In still another embodiment, multiple copies of these miRs or combinations may be used.

Conversely, miRNA binding sites can be removed from nucleic acid molecule (e.g., RNA, e.g., mRNA) sequences in which they naturally occur in order to increase protein expression in specific tissues. For example, a binding site for a specific miRNA can be removed from a nucleic acid molecule (e.g., RNA, e.g., mRNA) to improve protein expression in tissues or cells containing the miRNA.

In one embodiment, a nucleic acid molecule (e.g., RNA, e.g., mRNA) of the disclosure can include at least one miRNA-binding site in the 5′UTR and/or 3′UTR in order to regulate cytotoxic or cytoprotective mRNA therapeutics to specific cells such as, but not limited to, normal and/or cancerous cells. In another embodiment, a nucleic acid molecule (e.g., RNA, e.g., mRNA) of the disclosure can include two, three, four, five, six, seven, eight, nine, ten, or more miRNA-binding sites in the 5′-UTR and/or 3′-UTR in order to regulate cytotoxic or cytoprotective mRNA therapeutics to specific cells such as, but not limited to, normal and/or cancerous cells.

Regulation of expression in multiple tissues can be accomplished through introduction or removal of one or more miRNA binding sites, e.g., one or more distinct miRNA binding sites. The decision whether to remove or insert a miRNA binding site can be made based on miRNA expression patterns and/or their profilings in tissues and/or cells in development and/or disease. Identification of miRNAs, miRNA binding sites, and their expression patterns and role in biology have been reported (e.g., Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/leu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety).

miRNAs and miRNA binding sites can correspond to any known sequence, including non-limiting examples described in U.S. Publication Nos. 2014/0200261, 2005/0261218, and 2005/0059005, each of which are incorporated herein by reference in their entirety. Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126).

Specifically, miRNAs are known to be differentially expressed in immune cells (also called hematopoietic cells), such as antigen presenting cells (APCs) (e.g., dendritic cells and monocytes), monocytes, monocytes, B lymphocytes, T lymphocytes, granulocytes, natural killer cells, etc. Immune cell specific miRNAs are involved in immunogenicity, autoimmunity, the immune response to infection, inflammation, as well as unwanted immune response after gene therapy and tissue/organ transplantation. Immune cell specific miRNAs also regulate many aspects of development, proliferation, differentiation and apoptosis of hematopoietic cells (immune cells). For example, miR-142 and miR-146 are exclusively expressed in immune cells, particularly abundant in myeloid dendritic cells. It has been demonstrated that the immune response to a nucleic acid molecule (e.g., RNA, e.g., mRNA) can be shut-off by adding miR-142 binding sites to the 3′-UTR of the polynucleotide, enabling more stable gene transfer in tissues and cells. miR-142 efficiently degrades exogenous nucleic acid molecules (e.g., RNA, e.g., mRNA) in antigen presenting cells and suppresses cytotoxic elimination of transduced cells (e.g., Annoni A et al., blood, 2009, 114, 5152-5161; Brown B D, et al., Nat med. 2006, 12(5), 585-591; Brown B D, et al., blood, 2007, 110(13): 4144-4152, each of which is incorporated herein by reference in its entirety).

An antigen-mediated immune response can refer to an immune response triggered by foreign antigens, which, when entering an organism, are processed by the antigen presenting cells and displayed on the surface of the antigen presenting cells. T cells can recognize the presented antigen and induce a cytotoxic elimination of cells that express the antigen.

Introducing a miR-142 binding site into the 5′UTR and/or 3′UTR of a nucleic acid molecule of the disclosure can selectively repress gene expression in antigen presenting cells through miR-142 mediated degradation, limiting antigen presentation in antigen presenting cells (e.g., dendritic cells) and thereby preventing antigen-mediated immune response after the delivery of the nucleic acid molecule (e.g., RNA, e.g., mRNA). The nucleic acid molecule (e.g., RNA, e.g., mRNA) is then stably expressed in target tissues or cells without triggering cytotoxic elimination.

In one embodiment, binding sites for miRNAs that are known to be expressed in immune cells, in particular, antigen presenting cells, can be engineered into a nucleic acid molecule (e.g., RNA, e.g., mRNA) of the disclosure to suppress the expression of the nucleic acid molecule (e.g., RNA, e.g., mRNA) in antigen presenting cells through miRNA mediated RNA degradation, subduing the antigen-mediated immune response. Expression of the nucleic acid molecule (e.g., RNA, e.g., mRNA) is maintained in non-immune cells where the immune cell specific miRNAs are not expressed. For example, in some embodiments, to prevent an immunogenic reaction against a liver specific protein, any miR-122 binding site can be removed and a miR-142 (and/or mirR-146) binding site can be engineered into the 5′UTR and/or 3′UTR of a nucleic acid molecule of the disclosure.

To further drive the selective degradation and suppression in APCs and macrophage, a nucleic acid molecule (e.g., RNA, e.g., mRNA) of the disclosure can include a further negative regulatory element in the 5′UTR and/or 3′UTR, either alone or in combination with miR-142 and/or miR-146 binding sites. As a non-limiting example, the further negative regulatory element is a Constitutive Decay Element (CDE).

Immune cell specific miRNAs include, but are not limited to, hsa-let-7a-2-3p, hsa-let-7a-3p, hsa-7a-5p, hsa-let-7c, hsa-let-7e-3p, hsa-let-7e-5p, hsa-let-7g-3p, hsa-let-7g-5p, hsa-let-7i-3p, hsa-let-7i-5p, miR-10a-3p, miR-10a-5p, miR-1184, hsa-let-7f-1-3p, hsa-let-7f-2-5p, hsa-let-7f-5p, miR-125b-1-3p, miR-125b-2-3p, miR-125b-5p, miR-1279, miR-130a-3p, miR-130a-5p, miR-132-3p, miR-132-5p, miR-142-3p, miR-142-5p, miR-143-3p, miR-143-5p, miR-146a-3p, miR-146a-5p, miR-146b-3p, miR-146b-5p, miR-147a, miR-147b, miR-148a-5p, miR-148a-3p, miR-150-3p, miR-150-5p, miR-151b, miR-155-3p, miR-155-5p, miR-15a-3p, miR-15a-5p, miR-15b-5p, miR-15b-3p, miR-16-1-3p, miR-16-2-3p, miR-16-5p, miR-17-5p, miR-181a-3p, miR-181a-5p, miR-181a-2-3p, miR-182-3p, miR-182-5p, miR-197-3p, miR-197-5p, miR-21-5p, miR-21-3p, miR-214-3p, miR-214-5p, miR-223-3p, miR-223-5p, miR-221-3p, miR-221-5p, miR-23b-3p, miR-23b-5p, miR-24-1-5p, miR-24-2-5p, miR-24-3p, miR-26a-1-3p, miR-26a-2-3p, miR-26a-5p, miR-26b-3p, miR-26b-5p, miR-27a-3p, miR-27a-5p, miR-27b-3p, miR-27b-5p, miR-28-3p, miR-28-5p, miR-2909, miR-29a-3p, miR-29a-5p, miR-29b-1-5p, miR-29b-2-5p, miR-29c-3p, miR-29c-5p, miR-30e-3p, miR-30e-5p, miR-331-5p, miR-339-3p, miR-339-5p, miR-345-3p, miR-345-5p, miR-346, miR-34a-3p, miR-34a-5p, miR-363-3p, miR-363-5p, miR-372, miR-377-3p, miR-377-5p, miR-493-3p, miR-493-5p, miR-542, miR-548b-5p, miR548c-5p, miR-548i, miR-548j, miR-548n, miR-574-3p, miR-598, miR-718, miR-935, miR-99a-3p, miR-99a-5p, miR-99b-3p, and miR-99b-5p. Furthermore, novel miRNAs can be identified in immune cell through micro-array hybridization and microtome analysis (e.g., Jima D D et al, Blood, 2010, 116:e118-e127; Vaz C et al., BMC Genomics, 2010, 11,288, the content of each of which is incorporated herein by reference in its entirety.)

miRNAs that are known to be expressed in the liver include, but are not limited to, miR-107, miR-122-3p, miR-122-5p, miR-1228-3p, miR-1228-5p, miR-1249, miR-129-5p, miR-1303, miR-151a-3p, miR-151a-5p, miR-152, miR-194-3p, miR-194-5p, miR-199a-3p, miR-199a-5p, miR-199b-3p, miR-199b-5p, miR-296-5p, miR-557, miR-581, miR-939-3p, and miR-939-5p, miRNA binding sites from any liver specific miRNA can be introduced to or removed from a nucleic acid molecule (e.g., RNA, e.g., mRNA) of the disclosure to regulate expression of the nucleic acid molecule (e.g., RNA, e.g., mRNA) in the liver. Liver specific miRNA binding sites can be engineered alone or further in combination with immune cell (e.g., APC) miRNA binding sites in a nucleic acid molecule (e.g., RNA, e.g., mRNA) of the disclosure. In one embodiment, miRNA binding sites that promote degradation of mRNAs by hepatocytes are present in an mRNA molecule agent.

miRNAs that are known to be expressed in the lung include, but are not limited to, let-7a-2-3p, let-7a-3p, let-7a-5p, miR-126-3p, miR-126-5p, miR-127-3p, miR-127-5p, miR-130a-3p, miR-130a-5p, miR-130b-3p, miR-130b-5p, miR-133a, miR-133b, miR-134, miR-18a-3p, miR-18a-5p, miR-18b-3p, miR-18b-5p, miR-24-1-5p, miR-24-2-5p, miR-24-3p, miR-296-3p, miR-296-5p, miR-32-3p, miR-337-3p, miR-337-5p, miR-381-3p, and miR-381-5p, miRNA binding sites from any lung specific miRNA can be introduced to or removed from a nucleic acid molecule (e.g., RNA, e.g., mRNA) of the disclosure to regulate expression of the nucleic acid molecule (e.g., RNA, e.g., mRNA) in the lung. Lung specific miRNA binding sites can be engineered alone or further in combination with immune cell (e.g., APC) miRNA binding sites in a nucleic acid molecule (e.g., RNA, e.g., mRNA) of the disclosure.

miRNAs that are known to be expressed in the heart include, but are not limited to, miR-1, miR-133a, miR-133b, miR-149-3p, miR-149-5p, miR-186-3p, miR-186-5p, miR-208a, miR-208b, miR-210, miR-296-3p, miR-320, miR-451a, miR-451b, miR-499a-3p, miR-499a-5p, miR-499b-3p, miR-499b-5p, miR-744-3p, miR-744-5p, miR-92b-3p, and miR-92b-5p, miRNA binding sites from any heart specific microRNA can be introduced to or removed from a nucleic acid molecule (e.g., RNA, e.g., mRNA) of the disclosure to regulate expression of the nucleic acid molecule (e.g., RNA, e.g., mRNA) in the heart. Heart specific miRNA binding sites can be engineered alone or further in combination with immune cell (e.g., APC) miRNA binding sites in a nucleic acid molecule (e.g., RNA, e.g., mRNA) of the disclosure.

miRNAs that are known to be expressed in the nervous system include, but are not limited to, miR-124-5p, miR-125a-3p, miR-125a-5p, miR-125b-1-3p, miR-125b-2-3p, miR-125b-5p, miR-1271-3p, miR-1271-5p, miR-128, miR-132-5p, miR-135a-3p, miR-135a-5p, miR-135b-3p, miR-135b-5p, miR-137, miR-139-5p, miR-139-3p, miR-149-3p, miR-149-5p, miR-153, miR-181c-3p, miR-181c-5p, miR-183-3p, miR-183-5p, miR-190a, miR-190b, miR-212-3p, miR-212-5p, miR-219-1-3p, miR-219-2-3p, miR-23a-3p, miR-23a-5p, miR-30a-5p, miR-30b-3p, miR-30b-5p, miR-30c-1-3p, miR-30c-2-3p, miR-30c-5p, miR-30d-3p, miR-30d-5p, miR-329, miR-342-3p, miR-3665, miR-3666, miR-380-3p, miR-380-5p, miR-383, miR-410, miR-425-3p, miR-425-5p, miR-454-3p, miR-454-5p, miR-483, miR-510, miR-516a-3p, miR-548b-5p, miR-548c-5p, miR-571, miR-7-1-3p, miR-7-2-3p, miR-7-5p, miR-802, miR-922, miR-9-3p, and miR-9-5p, miRNAs enriched in the nervous system further include those specifically expressed in neurons, including, but not limited to, miR-132-3p, miR-132-3p, miR-148b-3p, miR-148b-5p, miR-151a-3p, miR-151a-5p, miR-212-3p, miR-212-5p, miR-320b, miR-320e, miR-323a-3p, miR-323a-5p, miR-324-5p, miR-325, miR-326, miR-328, miR-922 and those specifically expressed in glial cells, including, but not limited to, miR-1250, miR-219-1-3p, miR-219-2-3p, miR-219-5p, miR-23a-3p, miR-23a-5p, miR-3065-3p, miR-3065-5p, miR-30e-3p, miR-30e-5p, miR-32-5p, miR-338-5p, and miR-657, miRNA binding sites from any CNS specific miRNA can be introduced to or removed from a nucleic acid molecule (e.g., RNA, e.g., mRNA) of the disclosure to regulate expression of the nucleic acid molecule (e.g., RNA, e.g., mRNA) in the nervous system. Nervous system specific miRNA binding sites can be engineered alone or further in combination with immune cell (e.g., APC) miRNA binding sites in a nucleic acid molecule (e.g., RNA, e.g., mRNA) of the disclosure.

miRNAs that are known to be expressed in the pancreas include, but are not limited to, miR-105-3p, miR-105-5p, miR-184, miR-195-3p, miR-195-5p, miR-196a-3p, miR-196a-5p, miR-214-3p, miR-214-5p, miR-216a-3p, miR-216a-5p, miR-30a-3p, miR-33a-3p, miR-33a-5p, miR-375, miR-7-1-3p, miR-7-2-3p, miR-493-3p, miR-493-5p, and miR-944, miRNA binding sites from any pancreas specific miRNA can be introduced to or removed from a nucleic acid molecule (e.g., RNA, e.g., mRNA) of the disclosure to regulate expression of the nucleic acid molecule (e.g., RNA, e.g., mRNA) in the pancreas. Pancreas specific miRNA binding sites can be engineered alone or further in combination with immune cell (e.g. APC) miRNA binding sites in a nucleic acid molecule (e.g., RNA, e.g., mRNA) of the disclosure.

miRNAs that are known to be expressed in the kidney include, but are not limited to, miR-122-3p, miR-145-5p, miR-17-5p, miR-192-3p, miR-192-5p, miR-194-3p, miR-194-5p, miR-20a-3p, miR-20a-5p, miR-204-3p, miR-204-5p, miR-210, miR-216a-3p, miR-216a-5p, miR-296-3p, miR-30a-3p, miR-30a-5p, miR-30b-3p, miR-30b-5p, miR-30c-1-3p, miR-30c-2-3p, miR30c-5p, miR-324-3p, miR-335-3p, miR-335-5p, miR-363-3p, miR-363-5p, and miR-562, miRNA binding sites from any kidney specific miRNA can be introduced to or removed from a nucleic acid molecule (e.g., RNA, e.g., mRNA) of the disclosure to regulate expression of the nucleic acid molecule (e.g., RNA, e.g., mRNA) in the kidney. Kidney specific miRNA binding sites can be engineered alone or further in combination with immune cell (e.g., APC) miRNA binding sites in a nucleic acid molecule (e.g., RNA, e.g., mRNA) of the disclosure.

miRNAs that are known to be expressed in the muscle include, but are not limited to, let-7g-3p, let-7g-5p, miR-1, miR-1286, miR-133a, miR-133b, miR-140-3p, miR-143-3p, miR-143-5p, miR-145-3p, miR-145-5p, miR-188-3p, miR-188-5p, miR-206, miR-208a, miR-208b, miR-25-3p, and miR-25-5p, miRNA binding sites from any muscle specific miRNA can be introduced to or removed from a nucleic acid molecule (e.g., RNA, e.g., mRNA) of the disclosure to regulate expression of the nucleic acid molecule (e.g., RNA, e.g., mRNA) in the muscle. Muscle specific miRNA binding sites can be engineered alone or further in combination with immune cell (e.g., APC) miRNA binding sites in a nucleic acid molecule (e.g., RNA, e.g., mRNA) of the disclosure.

miRNAs are also differentially expressed in different types of cells, such as, but not limited to, endothelial cells, epithelial cells, and adipocytes.

miRNAs that are known to be expressed in endothelial cells include, but are not limited to, let-7b-3p, let-7b-5p, miR-100-3p, miR-100-5p, miR-101-3p, miR-101-5p, miR-126-3p, miR-126-5p, miR-1236-3p, miR-1236-5p, miR-130a-3p, miR-130a-5p, miR-17-5p, miR-17-3p, miR-18a-3p, miR-18a-5p, miR-19a-3p, miR-19a-5p, miR-19b-1-5p, miR-19b-2-5p, miR-19b-3p, miR-20a-3p, miR-20a-5p, miR-217, miR-210, miR-21-3p, miR-21-5p, miR-221-3p, miR-221-5p, miR-222-3p, miR-222-5p, miR-23a-3p, miR-23a-5p, miR-296-5p, miR-361-3p, miR-361-5p, miR-421, miR-424-3p, miR-424-5p, miR-513a-5p, miR-92a-1-5p, miR-92a-2-5p, miR-92a-3p, miR-92b-3p, and miR-92b-5p. Many novel miRNAs are discovered in endothelial cells from deep-sequencing analysis (e.g., Voellenkle C et al., RNA, 2012, 18, 472-484, herein incorporated by reference in its entirety), miRNA binding sites from any endothelial cell specific miRNA can be introduced to or removed from a nucleic acid molecule (e.g., RNA, e.g., mRNA) of the disclosure to regulate expression of the nucleic acid molecule (e.g., RNA, e.g., mRNA) in the endothelial cells.

miRNAs that are known to be expressed in epithelial cells include, but are not limited to, let-7b-3p, let-7b-5p, miR-1246, miR-200a-3p, miR-200a-5p, miR-200b-3p, miR-200b-5p, miR-200c-3p, miR-200c-5p, miR-338-3p, miR-429, miR-451a, miR-451b, miR-494, miR-802 and miR-34a, miR-34b-5p, miR-34c-5p, miR-449a, miR-449b-3p, miR-449b-5p specific in respiratory ciliated epithelial cells, let-7 family, miR-133a, miR-133b, miR-126 specific in lung epithelial cells, miR-382-3p, miR-382-5p specific in renal epithelial cells, and miR-762 specific in corneal epithelial cells, miRNA binding sites from any epithelial cell specific miRNA can be introduced to or removed from a nucleic acid molecule (e.g., RNA, e.g., mRNA) of the disclosure to regulate expression of the nucleic acid molecule (e.g., RNA, e.g., mRNA) in the epithelial cells.

In addition, a large group of miRNAs are enriched in embryonic stem cells, controlling stem cell self-renewal as well as the development and/or differentiation of various cell lineages, such as neural cells, cardiac, hematopoietic cells, skin cells, osteogenic cells and muscle cells (e.g., Kuppusamy K T et al., Curr. Mol Med, 2013, 13(5), 757-764; Vidigal J A and Ventura A, Semin Cancer Biol. 2012, 22(5-6), 428-436; Goff L A et al., PLoS One, 2009, 4:e7192; Morin R D et al., Genome Res, 2008, 18, 610-621; Yoo J K et al., Stem Cells Dev. 2012, 21(11), 2049-2057, each of which is herein incorporated by reference in its entirety), miRNAs abundant in embryonic stem cells include, but are not limited to, let-7a-2-3p, let-a-3p, let-7a-5p, let7d-3p, let-7d-5p, miR-103a-2-3p, miR-103a-5p, miR-106b-3p, miR-106b-5p, miR-1246, miR-1275, miR-138-1-3p, miR-138-2-3p, miR-138-5p, miR-154-3p, miR-154-5p, miR-200c-3p, miR-200c-5p, miR-290, miR-301a-3p, miR-301a-5p, miR-302a-3p, miR-302a-5p, miR-302b-3p, miR-302b-5p, miR-302c-3p, miR-302c-5p, miR-302d-3p, miR-302d-5p, miR-302e, miR-367-3p, miR-367-5p, miR-369-3p, miR-369-5p, miR-370, miR-371, miR-373, miR-380-5p, miR-423-3p, miR-423-5p, miR-486-5p, miR-520c-3p, miR-548e, miR-548f, miR-548g-3p, miR-548g-5p, miR-548i, miR-548k, miR-5481, miR-548m, miR-548n, miR-548o-3p, miR-548o-5p, miR-548p, miR-664a-3p, miR-664a-5p, miR-664b-3p, miR-664b-5p, miR-766-3p, miR-766-5p, miR-885-3p, miR-885-5p, miR-93-3p, miR-93-5p, miR-941, miR-96-3p, miR-96-5p, miR-99b-3p and miR-99b-5p. Many predicted novel miRNAs are discovered by deep sequencing in human embryonic stem cells (e.g., Morin R D et al., Genome Res, 2008, 18, 610-621; Goff L A et al., PLoS One, 2009, 4:e7192; Bar M et al., Stem cells, 2008, 26, 2496-2505, the content of each of which is incorporated herein by reference in its entirety).

In some embodiments, the binding sites of embryonic stem cell specific miRNAs can be included in or removed from the 3′UTR of a nucleic acid molecule (e.g., RNA, e.g., mRNA) of the disclosure to modulate the development and/or differentiation of embryonic stem cells, to inhibit the senescence of stem cells in a degenerative condition (e.g. degenerative diseases), or to stimulate the senescence and apoptosis of stem cells in a disease condition (e.g. cancer stem cells).

Many miRNA expression studies are conducted to profile the differential expression of miRNAs in various cancer cells/tissues and other diseases. Some miRNAs are abnormally over-expressed in certain cancer cells and others are under-expressed. For example, miRNAs are differentially expressed in cancer cells (WO2008/154098, US2013/0059015, US2013/0042333, WO2011/157294); cancer stem cells (US2012/0053224); pancreatic cancers and diseases (US2009/0131348, US2011/0171646, US2010/0286232, U.S. Pat. No. 8,389,210); asthma and inflammation (U.S. Pat. No. 8,415,096); prostate cancer (US2013/0053264); hepatocellular carcinoma (WO2012/151212, US2012/0329672, WO2008/054828, U.S. Pat. No. 8,252,538); lung cancer cells (WO2011/076143, WO2013/033640, WO2009/070653, US2010/0323357); cutaneous T cell lymphoma (WO2013/011378); colorectal cancer cells (WO2011/0281756, WO2011/076142); cancer positive lymph nodes (WO2009/100430, US2009/0263803); nasopharyngeal carcinoma (EP2112235); chronic obstructive pulmonary disease (US2012/0264626, US2013/0053263); thyroid cancer (WO2013/066678); ovarian cancer cells (US2012/0309645, WO2011/095623); breast cancer cells (WO2008/154098, WO2007/081740, US2012/0214699), leukemia and lymphoma (WO2008/073915, US2009/0092974, US2012/0316081, US2012/0283310, WO2010/018563), the content of each of which is incorporated herein by reference in its entirety.

As a non-limiting example, miRNA binding sites for miRNAs that are over-expressed in certain cancer and/or tumor cells can be removed from the 3′UTR of a nucleic acid molecule (e.g., RNA, e.g., mRNA) of the disclosure, restoring the expression suppressed by the over-expressed miRNAs in cancer cells, thus ameliorating the corresponsive biological function, for instance, transcription stimulation and/or repression, cell cycle arrest, apoptosis and cell death. Normal cells and tissues, wherein miRNAs expression is not up-regulated, will remain unaffected.

miRNA can also regulate complex biological processes such as angiogenesis (e.g., miR-132) (Anand and Cheresh Curr Opin Hematol 2011 18:171-176). In the nucleic acid molecules (e.g., RNA, e.g., mRNA) of the disclosure, miRNA binding sites that are involved in such processes can be removed or introduced, in order to tailor the expression of the nucleic acid molecules (e.g., RNA, e.g., mRNA) to biologically relevant cell types or relevant biological processes. In this context, the nucleic acid molecules (e.g., RNA, e.g., mRNA) of the disclosure are defined as auxotrophic polynucleotides.

In some embodiments, the therapeutic window and/or differential expression (e.g., tissue-specific expression) of a polypeptide of the disclosure may be altered by incorporation of a miRNA binding site into a nucleic acid molecule (e.g., RNA, e.g., mRNA) encoding the polypeptide. In one example, a nucleic acid molecule (e.g., RNA, e.g., mRNA) may include one or more miRNA binding sites that are bound by miRNAs that have higher expression in one tissue type as compared to another. In another example, a nucleic acid molecule (e.g., RNA, e.g., mRNA) may include one or more miRNA binding sites that are bound by miRNAs that have lower expression in a cancer cell as compared to a non-cancerous cell of the same tissue of origin. When present in a cancer cell that expresses low levels of such an miRNA, the polypeptide encoded by the nucleic acid molecule (e.g., RNA, e.g., mRNA) typically will show increased expression.

Liver cancer cells (e.g., hepatocellular carcinoma cells) typically express low levels of miR-122 as compared to normal liver cells. Therefore, a nucleic acid molecule (e.g., RNA, e.g., mRNA) encoding a polypeptide that includes at least one miR-122 binding site (e.g., in the 3′-UTR of the mRNA) will typically express comparatively low levels of the polypeptide in normal liver cells and comparatively high levels of the polypeptide in liver cancer cells. If the polypeptide is able to induce immunogenic cell death, this can cause preferential immunogenic cell killing of liver cancer cells (e.g., hepatocellular carcinoma cells) as compared to normal liver cells.

In some embodiments, the nucleic acid molecule (e.g., RNA, e.g., mRNA) includes at least one miR-122 binding site, at least two miR-122 binding sites, at least three miR-122 binding sites, at least four miR-122 binding sites, or at least five miR-122 binding sites. In one aspect, the miRNA binding site binds miR-122 or is complementary to miR-122. In another aspect, the miRNA binding site binds to miR-122-3p or miR-122-5p. In a particular aspect, the miRNA binding site comprises a nucleotide sequence at least 80%, at least 85%, at least 90%, at least 95%, or 100% identical to SEQ ID NO: 159, wherein the miRNA binding site binds to miR-122. In another particular aspect, the miRNA binding site comprises a nucleotide sequence at least 80%, at least 85%, at least 90%, at least 95%, or 100% identical to SEQ ID NO: 161, wherein the miRNA binding site binds to miR-122. These sequences are shown below in Table 14.

In some embodiments, a nucleic acid molecule (e.g., RNA, e.g., mRNA) of the disclosure comprises a miRNA binding site, wherein the miRNA binding site comprises one or more nucleotide sequences selected from Table 14, including one or more copies of any one or more of the miRNA binding site sequences. In some embodiments, a nucleic acid molecule (e.g., RNA, e.g., mRNA) of the disclosure further comprises at least one, two, three, four, five, six, seven, eight, nine, ten, or more of the same or different miRNA binding sites selected from Table 14, including any combination thereof. In some embodiments, the miRNA binding site binds to miR-142 or is complementary to miR-142. In some embodiments, the miR-142 comprises SEQ ID NO: 177. In some embodiments, the miRNA binding site binds to miR-142-3p or miR-142-5p. In some embodiments, the miR-142-3p binding site comprises SEQ ID NO: 179. In some embodiments, the miR-142-5p binding site comprises SEQ ID NO: 181. In some embodiments, the miRNA binding site comprises a nucleotide sequence at least 80%, at least 85%, at least 90%, at least 95%, or 100% identical to SEQ ID NO: 179 or SEQ ID NO: 181. In some embodiments, the miRNA binding site comprises a nucleotide sequence at least 80%, at least 85%, at least 90%, at least 95%, or 100% identical to SEQ ID NO: 179 or SEQ ID NO: 181.

In some embodiments, the miRNA binding site binds to miR-122 or is complementary to miR-122. In some embodiments, the miR-122 comprises SEQ ID NO: 3006. In some embodiments, the miRNA binding site binds to miR-122-3p or miR-122-5p. In some embodiments, the miR-122-3p binding site comprises SEQ ID NO: 159. In some embodiments, the miR-122-5p binding site comprises SEQ ID NO: 161. In some embodiments, the miRNA binding site comprises a nucleotide sequence at least 80%, at least 85%, at least 90%, at least 95%, or 100% identical to SEQ ID NO: 159 or SEQ ID NO: 161. In some embodiments, the miRNA binding site comprises a nucleotide sequence at least 80%, at least 85%, at least 90%, at least 95%, or 100% identical to SEQ ID NO: 169 or SEQ ID NO: 161.

TABLE 14
Representative microRNAs and microRNA binding sites

| SEQ ID | | |
|---|---|---|
| NO. | Description | Sequence |
| 177 | miR-142 | GACAGUGCAGUCACCCAUAAAGUAGAAAGCACUAC |
| | | UAACAGCACUGGAGGGUGUAGUGUUUCCUACUUU |
| | | AUGGAUGAGUGUACUGUG |
| 178 | miR-142-3p | UGUAGUGUUUCCUACUUUAUGGA |
| 179 | miR-142-3p | UCCAUAAAGUAGGAAACACUACA |
| | binding site | |
| 180 | miR-142-5p | CAUAAAGUAGAAAGCACUACU |
| 181 | miR-142-5p | AGUAGUGCUUUCUACUUUAUG |
| | binding site | |
| 157 | miR-122 | CCUUAGCAGAGCUGUGGAGUGUGACAAUGGUGUU |
| | | UGUGUCUAAACUAUCAAACGCCAUUAUCACACUAA |
| | | AUAGCUACUGCUAGGC |
| 158 | miR-122-3p | AACGCCAUUAUCACACUAAAUA |
| 159 | miR-122-3p | UAUUUAGUGUGAUAAUGGCGUU |
| | binding site | |
| 160 | miR-122-5p | UGGAGUGUGACAAUGGUGUUUG |
| 161 | miR-122-5p | CAAACACCAUUGUCACACUCCA |
| | binding site | |

In some embodiments, a miRNA binding site is inserted in the nucleic acid molecule (e.g., RNA, e.g., mRNA) of the disclosure in any position of the nucleic acid molecule (e.g., RNA, e.g., mRNA) (e.g., the 5′UTR and/or 3′UTR). In some embodiments, the 5′UTR comprises a miRNA binding site. In some embodiments, the 3′UTR comprises a miRNA binding site. In some embodiments, the 5′UTR and the 3′UTR comprise a miRNA binding site. The insertion site in the nucleic acid molecule (e.g., RNA, e.g., mRNA) can be anywhere in the nucleic acid molecule (e.g., RNA, e.g., mRNA) as long as the insertion of the miRNA binding site in the nucleic acid molecule (e.g., RNA, e.g., mRNA) does not interfere with the translation of a functional polypeptide in the absence of the corresponding miRNA; and in the presence of the miRNA, the insertion of the miRNA binding site in the nucleic acid molecule (e.g., RNA, e.g., mRNA) and the binding of the miRNA binding site to the corresponding miRNA are capable of degrading the polynucleotide or preventing the translation of the nucleic acid molecule (e.g., RNA, e.g., mRNA).

In some embodiments, a miRNA binding site is inserted in at least about 30 nucleotides downstream from the stop codon of an ORF in a nucleic acid molecule (e.g., RNA, e.g., mRNA) of the disclosure comprising the ORF. In some embodiments, a miRNA binding site is inserted in at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides downstream from the stop codon of an ORF in a polynucleotide of the disclosure. In some embodiments, a miRNA binding site is inserted in about 10 nucleotides to about 100 nucleotides, about 20 nucleotides to about 90 nucleotides, about 30 nucleotides to about 80 nucleotides, about 40 nucleotides to about 70 nucleotides, about 50 nucleotides to about 60 nucleotides, about 45 nucleotides to about 65 nucleotides downstream from the stop codon of an ORF in a nucleic acid molecule (e.g., RNA, e.g., mRNA) of the disclosure.

miRNA gene regulation can be influenced by the sequence surrounding the miRNA such as, but not limited to, the species of the surrounding sequence, the type of sequence (e.g., heterologous, homologous, exogenous, endogenous, or artificial), regulatory elements in the surrounding sequence and/or structural elements in the surrounding sequence. The miRNA can be influenced by the 5′UTR and/or 3′UTR. As a non-limiting example, a non-human 3′UTR can increase the regulatory effect of the miRNA sequence on the expression of a polypeptide of interest compared to a human 3′UTR of the same sequence type.

In one embodiment, other regulatory elements and/or structural elements of the 5′UTR can influence miRNA mediated gene regulation. One example of a regulatory element and/or structural element is a structured IRES (Internal Ribosome Entry Site) in the 5′UTR, which is necessary for the binding of translational elongation factors to initiate protein translation. EIF4A2 binding to this secondarily structured element in the 5′-UTR is necessary for miRNA mediated gene expression (Meijer H A et al., Science, 2013, 340, 82-85, herein incorporated by reference in its entirety). The nucleic acid molecules (e.g., RNA, e.g., mRNA) of the disclosure can further include this structured 5′UTR in order to enhance microRNA mediated gene regulation.

At least one miRNA binding site can be engineered into the 3′UTR of a polynucleotide of the disclosure. In this context, at least two, at least three, at least four, at least five, at least six, at least seven, at least eight, at least nine, at least ten, or more miRNA binding sites can be engineered into a 3′UTR of a nucleic acid molecule (e.g., RNA, e.g., mRNA) of the disclosure. For example, 1 to 10, 1 to 9, 1 to 8, 1 to 7, 1 to 6, 1 to 5, 1 to 4, 1 to 3, 2, or 1 miRNA binding sites can be engineered into the 3′UTR of a nucleic acid molecule (e.g., RNA, e.g., mRNA) of the disclosure. In one embodiment, miRNA binding sites incorporated into a nucleic acid molecule (e.g., RNA, e.g., mRNA) of the disclosure can be the same or can be different miRNA sites. A combination of different miRNA binding sites incorporated into a nucleic acid molecule (e.g., RNA, e.g., mRNA) of the disclosure can include combinations in which more than one copy of any of the different miRNA sites are incorporated. In another embodiment, miRNA binding sites incorporated into a nucleic acid molecule (e.g., RNA, e.g., mRNA) of the disclosure can target the same or different tissues in the body. As a non-limiting example, through the introduction of tissue-, cell-type-, or disease-specific miRNA binding sites in the 3′-UTR of a nucleic acid molecule (e.g., RNA, e.g., mRNA) of the disclosure, the degree of expression in specific cell types (e.g., hepatocytes, myeloid cells, endothelial cells, cancer cells, etc.) can be reduced.

In one embodiment, a miRNA binding site can be engineered near the 5′ terminus of the 3′UTR, about halfway between the 5′ terminus and 3′ terminus of the 3′UTR and/or near the 3′ terminus of the 3′UTR in a nucleic acid molecule (e.g., RNA, e.g., mRNA) of the disclosure. As a non-limiting example, a miRNA binding site can be engineered near the 5′ terminus of the 3′UTR and about halfway between the 5′ terminus and 3′ terminus of the 3′UTR. As another non-limiting example, a miRNA binding site can be engineered near the 3′ terminus of the 3′UTR and about halfway between the 5′ terminus and 3′ terminus of the 3′UTR. As yet another non-limiting example, a miRNA binding site can be engineered near the 5′ terminus of the 3′UTR and near the 3′ terminus of the 3′UTR.

In another embodiment, a 3′UTR can comprise 1, 2, 3, 4, 5, 6, 7, 8, 9, or 10 miRNA binding sites. The miRNA binding sites can be complementary to a miRNA, miRNA seed sequence, and/or miRNA sequences flanking the seed sequence.

In one embodiment, a nucleic acid molecule (e.g., RNA, e.g., mRNA) of the disclosure can be engineered to include more than one miRNA site expressed in different tissues or different cell types of a subject. As a non-limiting example, a nucleic acid molecule (e.g., RNA, e.g., mRNA) of the disclosure can be engineered to include miR-192 and miR-122 to regulate expression of the nucleic acid molecule (e.g., RNA, e.g., mRNA) in the liver and kidneys of a subject. In another embodiment, a nucleic acid molecule (e.g., RNA, e.g., mRNA) of the disclosure can be engineered to include more than one miRNA site for the same tissue.

In some embodiments, the therapeutic window and or differential expression associated with the polypeptide encoded by a nucleic acid molecule (e.g., RNA, e.g., mRNA) of the disclosure can be altered with a miRNA binding site. For example, a nucleic acid molecule (e.g., RNA, e.g., mRNA) encoding a polypeptide that provides a death signal can be designed to be more highly expressed in cancer cells by virtue of the miRNA signature of those cells. Where a cancer cell expresses a lower level of a particular miRNA, the nucleic acid molecule (e.g., RNA, e.g., mRNA) encoding the binding site for that miRNA (or miRNAs) would be more highly expressed. Hence, the polypeptide that provides a death signal triggers or induces cell death in the cancer cell. Neighboring noncancer cells, harboring a higher expression of the same miRNA would be less affected by the encoded death signal as the polynucleotide would be expressed at a lower level due to the effects of the miRNA binding to the binding site or “sensor” encoded in the 3′UTR. Conversely, cell survival or cytoprotective signals can be delivered to tissues containing cancer and non-cancerous cells where a miRNA has a higher expression in the cancer cells—the result being a lower survival signal to the cancer cell and a larger survival signal to the normal cell. Multiple nucleic acid molecule (e.g., RNA, e.g., mRNA) can be designed and administered having different signals based on the use of miRNA binding sites as described herein.

In some embodiments, the expression of a nucleic acid molecule (e.g., RNA, e.g., mRNA) of the disclosure can be controlled by incorporating at least one sensor sequence in the polynucleotide and formulating the nucleic acid molecule (e.g., RNA, e.g., mRNA) for administration. As a non-limiting example, a nucleic acid molecule (e.g., RNA, e.g., mRNA) of the disclosure can be targeted to a tissue or cell by incorporating a miRNA binding site and formulating the nucleic acid molecule (e.g., RNA, e.g., mRNA) in a lipid nanoparticle comprising a cationic lipid, including any of the lipids described herein.

A nucleic acid molecule (e.g., RNA, e.g., mRNA) of the disclosure can be engineered for more targeted expression in specific tissues, cell types, or biological conditions based on the expression patterns of miRNAs in the different tissues, cell types, or biological conditions. Through introduction of tissue-specific miRNA binding sites, a nucleic acid molecule (e.g., RNA, e.g., mRNA) of the disclosure can be designed for optimal protein expression in a tissue or cell, or in the context of a biological condition.

In some embodiments, a nucleic acid molecule (e.g., RNA, e.g., mRNA) of the disclosure can be designed to incorporate miRNA binding sites that either have 100% identity to known miRNA seed sequences or have less than 100% identity to miRNA seed sequences. In some embodiments, a nucleic acid molecule (e.g., RNA, e.g., mRNA) of the disclosure can be designed to incorporate miRNA binding sites that have at least: 60%, 65%, 70%, 75%, 80%, 85%, 90%, 95%, 96%, 97%, 98%, or 99% identity to known miRNA seed sequences. The miRNA seed sequence can be partially mutated to decrease miRNA binding affinity and as such result in reduced downmodulation of the nucleic acid molecule (e.g., RNA, e.g., mRNA). In essence, the degree of match or mis-match between the miRNA binding site and the miRNA seed can act as a rheostat to more finely tune the ability of the miRNA to modulate protein expression. In addition, mutation in the non-seed region of a miRNA binding site can also impact the ability of a miRNA to modulate protein expression.

In one embodiment, a miRNA sequence can be incorporated into the loop of a stem loop.

In another embodiment, a miRNA seed sequence can be incorporated in the loop of a stem loop and a miRNA binding site can be incorporated into the 5′ or 3′ stem of the stem loop.

In one embodiment, a translation enhancer element (TEE) can be incorporated on the 5′end of the stem of a stem loop and a miRNA seed can be incorporated into the stem of the stem loop. In another embodiment, a TEE can be incorporated on the 5′ end of the stem of a stem loop, a miRNA seed can be incorporated into the stem of the stem loop and a miRNA binding site can be incorporated into the 3′ end of the stem or the sequence after the stem loop. The miRNA seed and the miRNA binding site can be for the same and/or different miRNA sequences.

In one embodiment, the incorporation of a miRNA sequence and/or a TEE sequence changes the shape of the stem loop region which can increase and/or decrease translation. (see e.g, Kedde et al., “A Pumilio-induced RNA structure switch in p27-3′UTR controls miR-221 and miR-22 accessibility.” Nature Cell Biology. 2010, incorporated herein by reference in its entirety).

In one embodiment, the 5′-UTR of a nucleic acid molecule (e.g., RNA, e.g., mRNA) of the disclosure can comprise at least one miRNA sequence. The miRNA sequence can be, but is not limited to, a 19 or 22 nucleotide sequence and/or a miRNA sequence without the seed.

In one embodiment the miRNA sequence in the 5′UTR can be used to stabilize a nucleic acid molecule (e.g., RNA, e.g., mRNA) of the disclosure described herein.

In another embodiment, a miRNA sequence in the 5′UTR of a nucleic acid molecule (e.g., RNA, e.g., mRNA) of the disclosure can be used to decrease the accessibility of the site of translation initiation such as, but not limited to a start codon. See, e.g., Matsuda et al., PLoS One. 2010 11(5):e15057; incorporated herein by reference in its entirety, which used antisense locked nucleic acid (LNA) oligonucleotides and exon-junction complexes (EJCs) around a start codon (−4 to +37 where the A of the AUG codons is +1) in order to decrease the accessibility to the first start codon (AUG). Matsuda showed that altering the sequence around the start codon with an LNA or EJC affected the efficiency, length and structural stability of a polynucleotide. A nucleic acid molecule (e.g., RNA, e.g., mRNA) of the disclosure can comprise a miRNA sequence, instead of the LNA or EJC sequence described by Matsuda et al, near the site of translation initiation in order to decrease the accessibility to the site of translation initiation. The site of translation initiation can be prior to, after or within the miRNA sequence. As a non-limiting example, the site of translation initiation can be located within a miRNA sequence such as a seed sequence or binding site. As another non-limiting example, the site of translation initiation can be located within a miR-122 sequence such as the seed sequence or the mir-122 binding site.

In some embodiments, a nucleic acid molecule (e.g., RNA, e.g., mRNA) of the disclosure can include at least one miRNA in order to dampen the antigen presentation by antigen presenting cells. The miRNA can be the complete miRNA sequence, the miRNA seed sequence, the miRNA sequence without the seed, or a combination thereof. As a non-limiting example, a miRNA incorporated into a nucleic acid molecule (e.g., RNA, e.g., mRNA) of the disclosure can be specific to the hematopoietic system. As another non-limiting example, a miRNA incorporated into a nucleic acid molecule (e.g., RNA, e.g., mRNA) of the disclosure to dampen antigen presentation is miR-142-3p.

In some embodiments, a nucleic acid molecule (e.g., RNA, e.g., mRNA) of the disclosure can include at least one miRNA in order to dampen expression of the encoded polypeptide in a tissue or cell of interest. As a non-limiting example, a nucleic acid molecule (e.g., RNA, e.g., mRNA) of the disclosure can include at least one miR-122 binding site in order to dampen expression of an encoded polypeptide of interest in the liver. As another non-limiting example a nucleic acid molecule (e.g., RNA, e.g., mRNA) of the disclosure can include at least one miR-142-3p binding site, miR-142-3p seed sequence, miR-142-3p binding site without the seed, miR-142-5p binding site, miR-142-5p seed sequence, miR-142-5p binding site without the seed, miR-146 binding site, miR-146 seed sequence and/or miR-146 binding site without the seed sequence.

In some embodiments, a nucleic acid molecule (e.g., RNA, e.g., mRNA) of the disclosure can comprise at least one miRNA binding site in the 3′UTR in order to selectively degrade mRNA therapeutics in the immune cells to subdue unwanted immunogenic reactions caused by therapeutic delivery. As a non-limiting example, the miRNA binding site can make a nucleic acid molecule (e.g., RNA, e.g., mRNA) of the disclosure more unstable in antigen presenting cells. Non-limiting examples of these miRNAs include mir-142-5p, mir-142-3p, mir-146a-5p, and mir-146-3p.

In one embodiment, a nucleic acid molecule (e.g., RNA, e.g., mRNA) of the disclosure comprises at least one miRNA sequence in a region of the nucleic acid molecule (e.g., RNA, e.g., mRNA) that can interact with a RNA binding protein.

In some embodiments, the nucleic acid molecule (e.g., RNA, e.g., mRNA) of the disclosure comprising (i) a sequence-optimized nucleotide sequence (e.g., an ORF) and (ii) a miRNA binding site (e.g., a miRNA binding site that binds to miR-142).

In some embodiments, the nucleic acid molecule (e.g., RNA, e.g., mRNA) of the disclosure comprises a uracil-modified sequence encoding a polypeptide disclosed herein and a miRNA binding site disclosed herein, e.g., a miRNA binding site that binds to miR-142. In some embodiments, the uracil-modified sequence encoding a polypeptide comprises at least one chemically modified nucleobase, e.g., 5-methoxyuracil. In some embodiments, at least 95% of a type of nucleobase (e.g., uracil) in a uracil-modified sequence encoding a polypeptide of the disclosure are modified nucleobases. In some embodiments, at least 95% of uracil in a uracil-modified sequence encoding a polypeptide is 5-methoxyuridine. In some embodiments, the nucleic acid molecule (e.g., RNA, e.g., mRNA) comprising a nucleotide sequence encoding a polypeptide disclosed herein and a miRNA binding site is formulated with a delivery agent.

### 3′-Stabilizing Region

In some embodiments, the mRNAs of the disclosure comprise a 3′-stabilizing region including one or more nucleosides (e.g., 1 to 500 nucleosides such as 1 to 200, 1 to 400, 1 to 10, 5 to 15, 10 to 20, 15 to 25, 20 to 30, 25 to 35, 30 to 40, 35 to 45, 40 to 50, 45 to 65, 50 to 70, 65 to 85, 70 to 90, 85 to 105, 90 to 110, 105 to 135, 120 to 150, 130 to 170, 150 to 200 or 1, 2, 3, 4, 5, 6, 7, 8, 9, 10, 20, 30, 40, 50, 60, 70, 80, 90, 100, 110, 120, 130, 140, 150, 160, 170, 180, 190, or 200 nucleosides). In some embodiments, the 3′-stabilizing region contains one or more alternative nucleosides having an alternative nucleobase, sugar, or backbone (e.g., a 2′-deoxynucleoside, a 3′-deoxynucleoside, a 2′,3′-dideoxynucleoside, a 2′-O-methylnucleoside, a 3′-O-methylnucleoside, a 3′-O-ethyl-nucleoside, 3′-arabinoside, an L-nucleoside, alpha-thio-2′-O-methyl-adenosine, 2′-fluoro-adenosine, arabino-adenosine, hexitol-adenosine, LNA-adenosine, PNA-adenosine, inverted thymidine, or 3′-azido-2′,3′-dideoxyadenosine). In some embodiments, the 3′-stabilizing region includes a plurality of alternative nucleosides. In some embodiments, the 3′-stabilizing region includes at least one non-nucleoside (e.g., an abasic ribose) at the 5′-terminus, the 3′-terminus, or at an internal position of the 3′-stabilizing region.

In some embodiments, the 3′-stablizing region consists of one nucleoside (e.g., a 2′-deoxynucleoside, a 3′-deoxynucleoside, a 2′,3′-dideoxynucleoside, a 2′-O-methylnucleoside, a 3′-O-methylnucleoside, a 3′-O-ethyl-nucleoside, 3′-arabinoside, an L-nucleoside, alpha-thio-2′-O-methyl-adenosine, 2′-fluoro-adenosine, arabino-adenosine, hexitol-adenosine, LNA-adenosine, PNA-adenosine, inverted thymidine, or 3′-azido-2′,3′-dideoxyadenosine).

In some embodiments, one or more nucleosides in the 3′-stabilizing region include the structure:

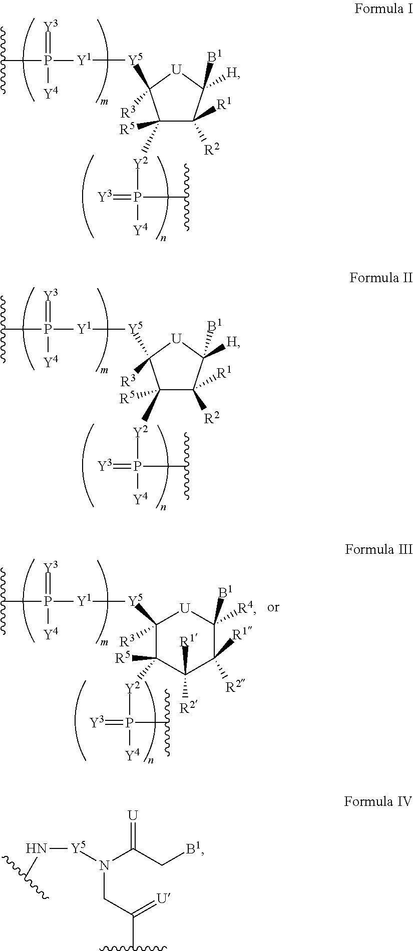

wherein B¹ is a nucleobase;
each U and U′ is, independently, O, S, N(R^{U})ₙᵤ, or C(R^{U})ₙᵤ, wherein nu is 1 or 2 (e.g., 1 for N(R^{U})ₙᵤ and 2 for C(R^{U})ₙᵤ) and each R^{U} is, independently, H, halo, or optionally substituted C₁-C₆ alkyl;
each of R¹, R^{1′}, R¹″, R², R^{2′}, R²″, R³, R⁴, and R⁵ is, independently, H, halo, hydroxy, thiol, optionally substituted C₁-C₆ alkyl, optionally substituted C₂-C₆ alkynyl, optionally substituted C₁-C₆ heteroalkyl, optionally substituted C₂-C₆ heteroalkenyl, optionally substituted C₂-C₆ heteroalkynyl, optionally substituted amino, azido, optionally substituted C₆-C₁₀ aryl; or R³ and/or R⁵ can join together with one of R¹, R^{1′}, R¹″, R², R^{2′}, or R²″ to form together with the carbons to which they are attached an optionally substituted C₃-C₁₀ carbocycle or an optionally substituted C₃-C₉ heterocyclyl;
each of m and n is independently, 0, 1, 2, 3, 4, or 5;
each of Y¹, Y², and Y³, is, independently, O, S, Se, —NR^{N1}—, optionally substituted C₁-C₆ alkylene, or optionally substituted C₁-C₆ heteroalkylene, wherein R^{N1} is H, optionally substituted C₁-C₆ alkyl, optionally substituted C₂-C₆ alkenyl, optionally substituted C₂-C₆ alkynyl, or optionally substituted C₆-C₁₀ aryl; and
each Y⁴ is, independently, H, hydroxy, protected hydroxy, halo, thiol, boranyl, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₂-C₆ heteroalkenyl, optionally substituted C₂-C₆ heteroalkynyl, or optionally substituted amino; and
Y⁵ is O, S, Se, optionally substituted C₁-C₆ alkylene, or optionally substituted C₁-C₆ heteroalkylene;
or is a salt thereof.

In some embodiments, the 3′-stabilizing region includes a plurality of adenosines. In some embodiments, all of the nucleosides of the 3′-stabilizing region are adenosines. In some embodiments, the 3′-stabilizing region includes at least one (e.g., at least two, at least three, at least four, at least five, at least six, at least seven, at least eight, at least nine, or at least ten) alternative nucleosides (e.g., an L-nucleoside such as L-adenosine, 2′-O-methyl-adenosine, alpha-thio-2′-O-methyl-adenosine, 2′-fluoro-adenosine, arabino-adenosine, hexitol-adenosine, LNA-adenosine, PNA-adenosine, or inverted thymidine). In some embodiments, the alternative nucleoside is an L-adenosine, a 2′-O-methyl-adenosine, or an inverted thymidine. In some embodiments, the 3′-stabilizing region includes a plurality of alternative nucleosides. In some embodiments, all of the nucleotides in the 3′-stabilizing region are alternative nucleosides. In some embodiments, the 3′-stabilizing region includes at least two different alternative nucleosides. In some embodiments, at least one alternative nucleoside is 2′-O-methyl-adenosine. In some embodiments, at least one alternative nucleoside is inverted thymidine. In some embodiments, at least one alternative nucleoside is 2′-O-methyl-adenosine, and at least one alternative nucleoside is inverted thymidine.

In some embodiments, the stabilizing region includes the structure:

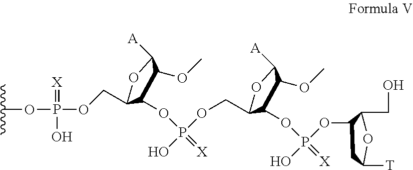

or a salt thereof;
wherein each X is, independently O or S; and
A represents adenine and T represents thymine.

In some embodiments, each X is O. In some embodiments, each X is S.

In some embodiments, all of the plurality of alternative nucleosides are the same (e.g., all of the alternative nucleosides are L-adenosine). In some embodiments, the 3′-stabilizing region includes ten nucleosides. In some embodiments, the 3′-stabilizing region includes eleven nucleosides. In some embodiments, the 3′-stabilizing region comprises at least five L-adenosines (e.g., at least ten L-adenosines, or at least twenty L-adenosines). In some embodiments, the 3′-stabilizing region consists of five L-adenosines. In some embodiments, the 3′-stabilizing region consists of ten L-adenosines. In some embodiments, the 3′-stabilizing region consists of twenty L-adenosines.

Further examples of 3′-stabilized regions are known in the art, e.g., as described in International Patent Publication Nos. WO2013/103659, WO2017/049275, and WO2017/049286, the 3′-stabilized regions of which are herein incorporated by references.

In some embodiments, the 5′-terminus of the 3′-stabilizing region is conjugated to the 3′-terminus of the 3′-UTR. In some embodiments, the 5′-terminus of the 3′-stabilizing region is conjugated to the 3′-terminus of the poly-A region. In some embodiments, the 5′-terminus of the 3′-stabilizing region is conjugated to the 3′-terminus of the poly-C region. In some embodiments of any of the foregoing polynucleotides, the 3′-stabilizing region includes the 3′-terminus of the polynucleotide.

In some embodiments, the 3′-stabilizing tail is conjugated to the remainder of the polynucleotide, e.g., at the 3′-terminus of the 3′-UTR or poly-A region via a phosphate linkage. In some embodiments, the phosphate linkage is a natural phosphate linkage. In some embodiments, the conjugation of the 3′-stabilizing tail and the remainder of the polynucleotide is produced via enzymatic or splint ligation.

In some embodiments, the 3′-stabilizing tail is conjugated to the remainder of the polynucleotide, e.g., at the 3′-terminus of the 3′-UTR or poly-A region via a chemical linkage. In some embodiments, the chemical linkage includes the structure of Formula VI:

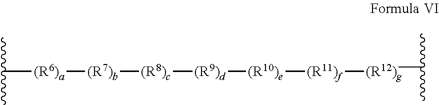

wherein a, b, c, e, f, and g are each, independently, 0 or 1;
d is 0, 1, 2, or 3;
each of R⁶, R⁸, R¹⁴, and R¹², is, independently, optionally substituted C₁-C₆ alkylene, optionally substituted C₁-C₆ heteroalkylene, optionally substituted C₂-C₆ alkenylene, optionally substituted C₂-C₆ alkynylene, or optionally substituted C₆-C₁₀ arylene, O, S, Se, and NR³;
R⁷ and R¹¹ are each, independently, carbonyl, thiocarbonyl, sulfonyl, or phosphoryl, wherein, if R⁷ is phosphoryl, —(R⁹)_{d}—is a bond, and e, f, and gare 0, then at least one of R⁶ or R⁸ is not 0; and if R¹¹ is phosphoryl, —(R⁹)_{d}—is a bond, and a, b, and c are 0, then at least one of R¹¹ or R¹² is not 0;
each R⁹ is optionally substituted C₁-C₁₀ alkylene, optionally substituted C₂-C₁₀ alkenylene, optionally substituted C₂-C₁₀ alkynylene, optionally substituted C₂-C₁₀ heterocyclylene, optionally substituted C₆-C₁₂ arylene, optionally substituted C₂-C₁₀₀ polyethylene glycolene, or optionally substituted C₁-C₁₀ heteroalkylene, or a bond linking (R₆)ₐ—(R⁷)_{b}—(R′)_{c} to (R¹⁰)ₑ-(R¹¹)_{f}-(R¹²)_{g}, wherein if —(R⁹)_{d}— is a bond, then at least one of a, b, c, e, f, or g is 1; and
R¹³ is hydrogen, optionally substituted C₁-C₄ alkyl, optionally substituted C₂-C₄ alkenyl, optionally substituted C₂-C₄ alkynyl, optionally substituted C₂-C₆ heterocyclyl, optionally substituted C₆-C₁₂ aryl, or optionally substituted C₁-C₇ heteroalkyl.

In some embodiments, the chemical linkage comprises the structure of Formula VII:

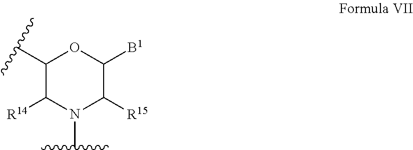

wherein B¹ is a nucleobase, hydrogen, halo, hydroxy, thiol, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₂-C₆ heteroalkenyl, optionally substituted C₂-C₆ heteroalkynyl, optionally substituted amino, azido, optionally substituted C₃-C₁₀ cycloalkyl, optionally substituted C₆-C₁₀ aryl, optionally substituted C₂-C₉ heterocycle; and
R¹⁴ and R¹⁵ are each, independently, hydrogen or hydroxy.

In some embodiments, the chemical linkage includes the structure:

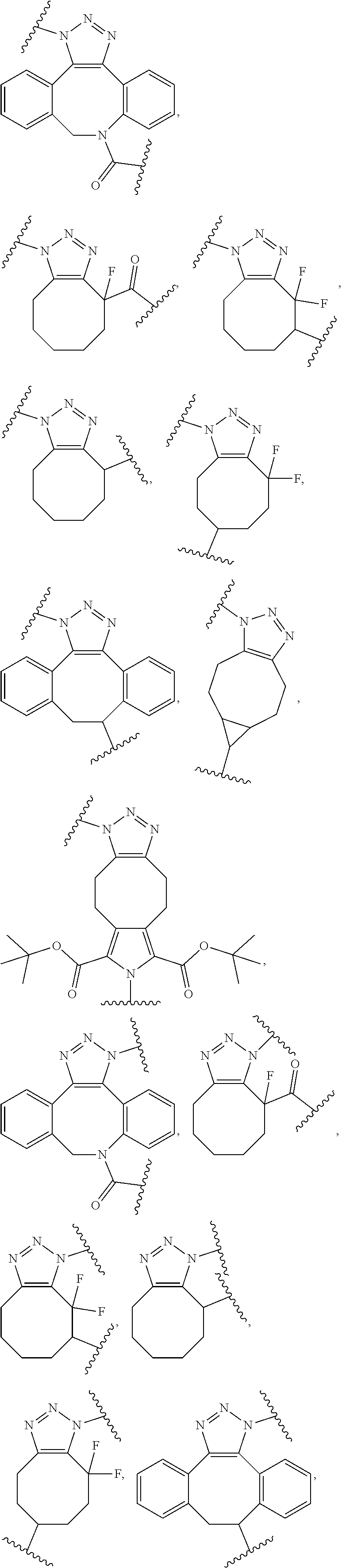

or an amide bond. Further examples of chemical linkages to conjugate 3′-stabilized regions to the remainder of the polynucleotide are known in the art, e.g., as described in International Patent Publication Nos. WO2017/049275 and WO2017/049286, the chemical linkers of which are herein incorporated by reference.

### Delivery Agents

### Lipid Compound

The present disclosure provides pharmaceutical compositions with advantageous properties. The lipid compositions described herein may be advantageously used in lipid nanoparticle compositions for the delivery of therapeutic and/or prophylactic agents, e.g., mRNAs, to mammalian cells or organs. For example, the lipids described herein have little or no immunogenicity. For example, the lipid compounds disclosed herein have a lower immunogenicity as compared to a reference lipid (e.g., MC3, KC2, or DLinDMA). For example, a formulation comprising a lipid disclosed herein and a therapeutic or prophylactic agent, e.g., mRNA, has an increased therapeutic index as compared to a corresponding formulation which comprises a reference lipid (e.g., MC3, KC2, or DLinDMA) and the same therapeutic or prophylactic agent.

In certain embodiments, the present application provides pharmaceutical compositions comprising:
(a) an mRNA comprising a nucleotide sequence encoding a polypeptide of interest; and
(b) a delivery agent.

### Lipid Nanoparticle Formulations

In some embodiments, nucleic acids of the invention (e.g. mRNA) are formulated in a lipid nanoparticle (LNP). Lipid nanoparticles typically comprise ionizable cationic lipid, non-cationic lipid, sterol and PEG lipid components along with the nucleic acid cargo of interest. The lipid nanoparticles of the invention can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575 and PCT/US2016/069491 all of which are incorporated by reference herein in their entirety.

Nucleic acids of the present disclosure (e.g. mRNA) are typically formulated in lipid nanoparticle. In some embodiments, the lipid nanoparticle comprises at least one ionizable cationic lipid, at least one non-cationic lipid, at least one sterol, and/or at least one polyethylene glycol (PEG)-modified lipid.

In some embodiments, the lipid nanoparticle comprises a molar ratio of 20-60% ionizable cationic lipid. For example, the lipid nanoparticle may comprise a molar ratio of 20-50%, 20-40%, 20-30%, 30-60%, 30-50%, 30-40%, 40-60%, 40-50%, or 50-60% ionizable cationic lipid. In some embodiments, the lipid nanoparticle comprises a molar ratio of 20%, 30%, 40%, 50, or 60% ionizable cationic lipid.

In some embodiments, the lipid nanoparticle comprises a molar ratio of 5-25% non-cationic lipid. For example, the lipid nanoparticle may comprise a molar ratio of 5-20%, 5-15%, 5-10%, 10-25%, 10-20%, 10-25%, 15-25%, 15-20%, or 20-25% non-cationic lipid. In some embodiments, the lipid nanoparticle comprises a molar ratio of 5%, 10%, 15%, 20%, or 25% non-cationic lipid.

In some embodiments, the lipid nanoparticle comprises a molar ratio of 25-55% sterol. For example, the lipid nanoparticle may comprise a molar ratio of 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% sterol. In some embodiments, the lipid nanoparticle comprises a molar ratio of 25%, 30%, 35%, 40%, 45%, 50%, or 55% sterol.

In some embodiments, the lipid nanoparticle comprises a molar ratio of 0.5-15% PEG-modified lipid. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15%. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-modified lipid.

In some embodiments, the lipid nanoparticle comprises a molar ratio of 20-60% ionizable cationic lipid, 5-25% non-cationic lipid, 25-55% sterol, and 0.5-15% PEG-modified lipid.

### Ionizable Lipids

In some aspects, the ionizable lipids of the present disclosure may be one or more of compounds of Formula (I):

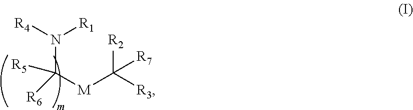

or their N-oxides, or salts or isomers thereof, wherein:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of hydrogen, a C₃₋₆ carbocycle, —(CH₂)ₙQ, —(CH₂)ₙCHQR,
-CHQR, -CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —N(R)S(O)₂R₈, —O(CH₂)˜OR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —OC(O)-M″-C(O)O—, —C(O)N(R′)—,
—N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₂ alkyl, C₂₋₁₈ alkenyl, —R*YR″, —YR″, and H;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, C₁, Br, and I; and
m is selected from 5, 6, 7, 8, 9, 10, 11, 12, and 13; and wherein when R₄ is —(CH₂)ₙQ, —(CH₂)ₙCHQR, -CHQR, or -CQ(R)₂, then (i) Q is not —N(R)₂ when n is 1, 2, 3, 4 or 5, or (ii) Q is not 5, 6, or 7-membered heterocycloalkyl when n is 1 or 2.

In certain embodiments, a subset of compounds of Formula (I) includes those of Formula (IA):

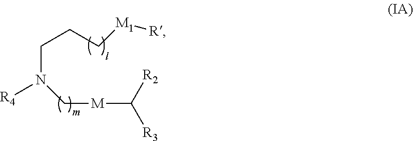

or its N-oxide, or a salt or isomer thereof, wherein 1 is selected from 1, 2, 3, 4, and 5; m is selected from 5, 6, 7, 8, and 9; M₁ is a bond or M′; R₄ is hydrogen, unsubstituted C₁₋₃ alkyl, or —(CH₂)ₙQ, in which Q is
   OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected
   from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (I) includes those of Formula (IB):

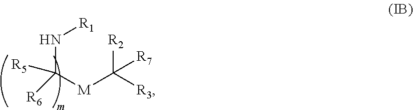

or its N-oxide, or a salt or isomer thereof in which all variables are as defined herein. For example, m is selected from 5, 6, 7, 8, and 9; R₄ is hydrogen, unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (I) includes those of Formula (II):

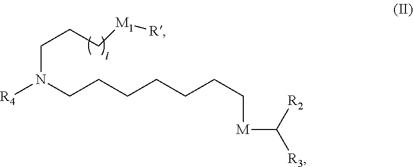

or its N-oxide, or a salt or isomer thereof, wherein 1 is selected from 1, 2, 3, 4, and 5; M₁ is a bond or M′; R₄ is hydrogen, unsubstituted C₁₋₃ alkyl, or —(CH₂)ₙQ, in which n is 2, 3, or 4, and Q is
OH, —NHC(S)N(R)₂, —NHC(O)N(R)₂, —N(R)C(O)R, —N(R)S(O)₂R, —N(R)R₈,
—NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In one embodiment, the compounds of Formula (I) are of Formula (IIa),

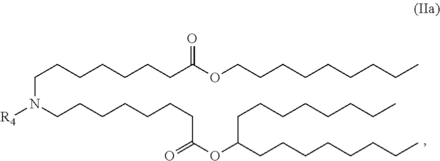

or their N-oxides, or salts or isomers thereof, wherein R₄ is as described herein.

In another embodiment, the compounds of Formula (I) are of Formula (IIb),

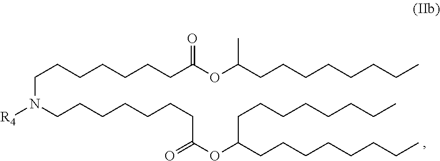

or their N-oxides, or salts or isomers thereof, wherein R₄ is as described herein.

In another embodiment, the compounds of Formula (I) are of Formula (IIc) or (IIe):

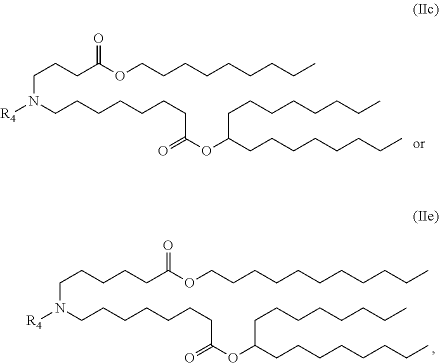

or their N-oxides, or salts or isomers thereof, wherein R₄ is as described herein.

In another embodiment, the compounds of Formula (I) are of Formula (IIf):

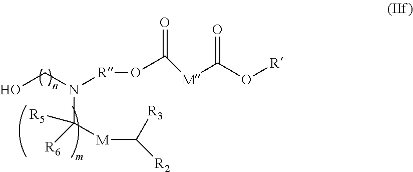

or their N-oxides, or salts or isomers thereof,
wherein M is —C(O)O— or —OC(O)—, M″ is C₁₋₆ alkyl or C₂₋₆ alkenyl, R₂ and R₃ are independently selected from the group consisting of C₅₋₁₄ alkyl and C₅₋₁₄ alkenyl, and n is selected from 2, 3, and 4.

In a further embodiment, the compounds of Formula (I) are of Formula (IId),

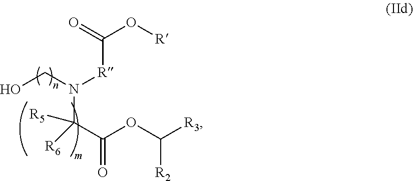

or their N-oxides, or salts or isomers thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In a further embodiment, the compounds of Formula (I) are of Formula (IIg),

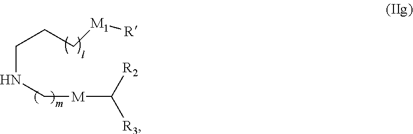

or their N-oxides, or salts or isomers thereof, wherein 1 is selected from 1, 2, 3, 4, and 5; m is selected from 5, 6, 7, 8, and 9; M₁ is a bond or M′; M and M′ are independently selected from
—C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R₂ and R₃ are independently selected from the group consisting of C₅₋₁₄ alkyl and C₅₋₁₄ alkenyl.

In some embodiments, the ionizable lipids are one or more of the compounds described in U.S. Application Nos. 62/220,091, 62/252,316, 62/253,433, 62/266,460, 62/333,557, 62/382,740, 62/393,940, 62/471,937, 62/471,949, 62/475,140, and 62/475,166, and PCT Application No. PCT/US2016/052352.

In some embodiments, the ionizable lipids are selected from Compounds 1-280 described in U.S. Application No. 62/475,166.

In some embodiments, the ionizable lipid is

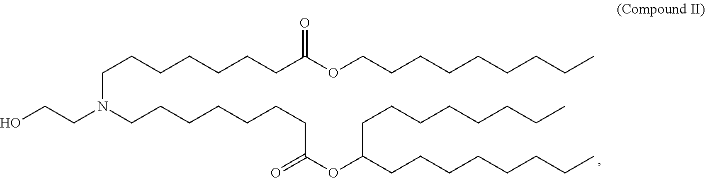 [SMILES: CCCCCCCCCOC(=O)CCCCCCCN(CCO)CCCCCCCC(=O)OC(CCCCCCCC)CCCCCCCC]

or a salt thereof.

In some embodiments, the ionizable lipid is

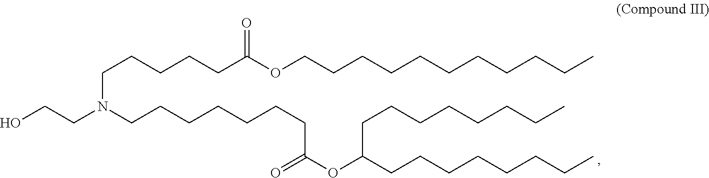 [SMILES: CCCCCCCCCCCOC(=O)CCCCCN(CCO)CCCCCCCC(=O)OC(CCCCCCCC)CCCCCCCC]

or a salt thereof.

In some embodiments, the ionizable lipid is

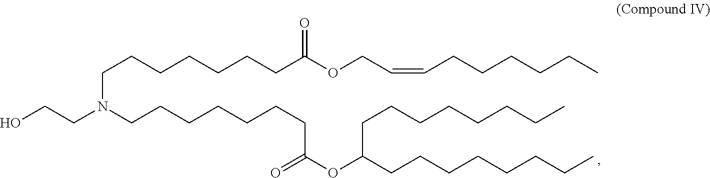 [SMILES: CCCCCC/C=C\COC(=O)CCCCCCCN(CCO)CCCCCCCC(=O)OC(CCCCCCCC)CCCCCCCC]

or a salt thereof.

In some embodiments, the ionizable lipid is

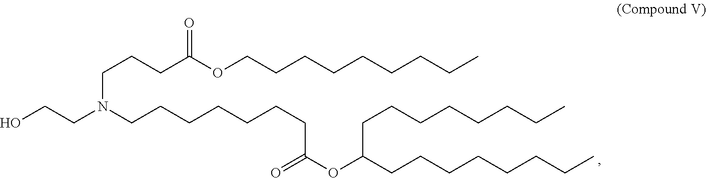 [SMILES: CCCCCCCCCOC(=O)CCCN(CCO)CCCCCCCC(=O)OC(CCCCCCCC)CCCCCCCC]

or a salt thereof.

The central amine moiety of a lipid according to Formula (I), (IA), (IB), (II), (IIa), (IIb), (IIc), (IId), (IIe), (IIf), or (IIg) may be protonated at a physiological pH. Thus, a lipid may have a positive or partial positive charge at physiological pH. Such lipids may be referred to as cationic or ionizable (amino) lipids. Lipids may also be zwitterionic, i.e., neutral molecules having both a positive and a negative charge.

In some aspects, the ionizable lipids of the present disclosure may be one or more of compounds of formula (III),

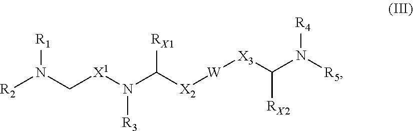

or salts or isomers thereof, wherein
W is

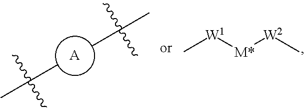

ring A is

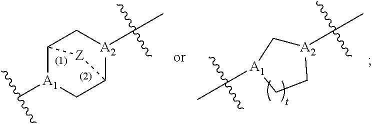

t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″; R_{X1} and R_{X2} are each independently H or C₁₋₃ alkyl;
each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;
M* is C₁-C₆ alkyl,
W¹ and W² are each independently selected from the group consisting of —O— and —N(R₆)—;
each R₆ is independently selected from the group consisting of H and C₁₋₅ alkyl;
X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)˜-OC(O)—, —C(O)O—(CH₂)ₙ—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H;
each R″ is independently selected from the group consisting of C₃₋₁₂ alkyl, C₃₋₁₂ alkenyl and —R*MR′; and
n is an integer from 1-6;
when ring A is

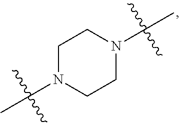 [SMILES: *N1CCN(*)CC1]

then
i) at least one of X¹, X², and X³ is not —CH₂—; and/or
ii) at least one of R₁, R₂, R₃, R₄, and R₅ is —R″MR′.

In some embodiments, the compound is of any of formulae (IIIa1)-(IIIa8):

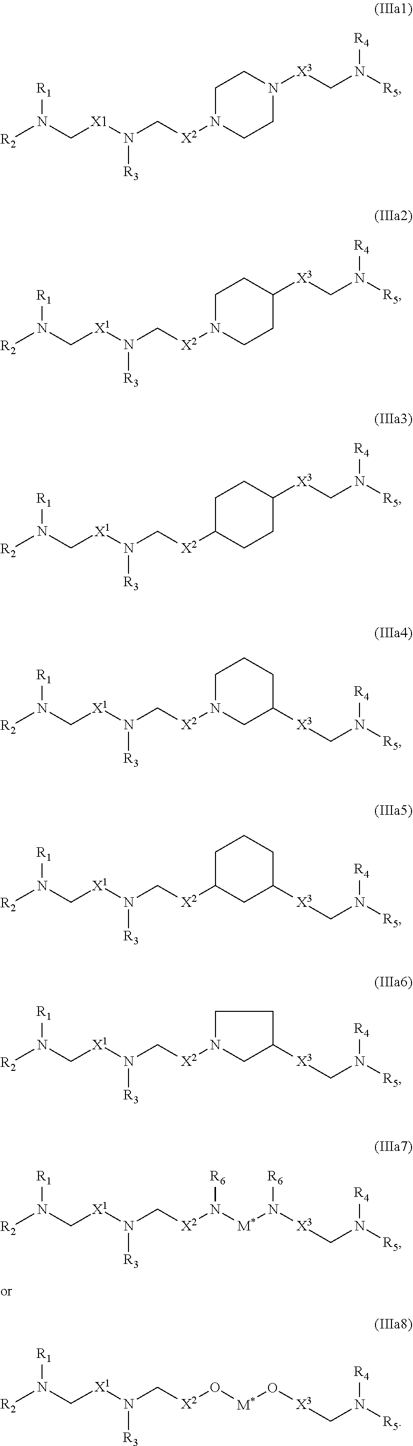

In some embodiments, the ionizable lipids are one or more of the compounds described in U.S. Application Nos. 62/271,146, 62/338,474, 62/413,345, and 62/519,826, and PCT Application No. PCT/US2016/068300.

In some embodiments, the ionizable lipids are selected from Compounds 1-156 described in U.S. Application No. 62/519,826.

In some embodiments, the ionizable lipids are selected from Compounds 1-16, 42-66, 68-76, and 78-156 described in U.S. Application No. 62/519,826.

In some embodiments, the ionizable lipid is

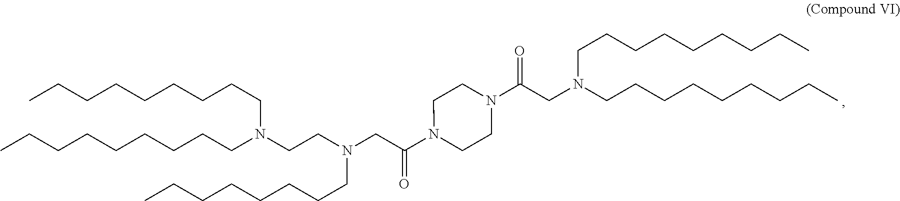 [SMILES: CCCCCCCCCN(CCCCCCCCC)CCN(CCCCCCCC)CC(=O)N1CCN(C(=O)CN(CCCCCCCCC)CCCCCCCCC)CC1]

or a salt thereof.

In some embodiments, the ionizable lipid is (Compound VII), or a salt thereof.

The central amine moiety of a lipid according to Formula (III), (IIIa1), (IIIa2), (IIIa3), (IIIa4), (IIIa5), (IIIa6), (IIIa7), or (IIIa8) may be protonated at a physiological pH. Thus, a lipid may have a positive or partial positive charge at physiological pH. Such lipids may be referred to as cationic or ionizable (amino)lipids. Lipids may also be zwitterionic, i.e., neutral molecules having both a positive and a negative charge.

### Phospholipids

The lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid of the invention comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In certain embodiments, a phospholipid useful or potentially useful in the present invention is an analog or variant of DSPC. In certain embodiments, a phospholipid useful or potentially useful in the present invention is a compound of Formula (IV):

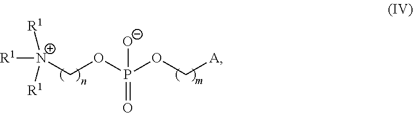

or a salt thereof, wherein:
each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the formula:

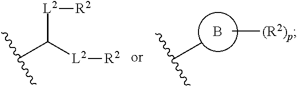

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or —NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, —C(O), C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), —NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2;
provided that the compound is not of the formula:

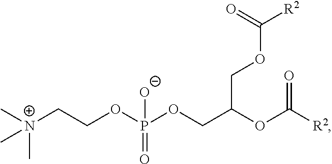

wherein each instance of R² is independently unsubstituted alkyl, unsubstituted alkenyl, or unsubstituted alkynyl.

In some embodiments, the phospholipids may be one or more of the phospholipids described in U.S. Application No. 62/520,530.

### (i) Phospholipid Head Modifications

In certain embodiments, a phospholipid useful or potentially useful in the present invention comprises a modified phospholipid head (e.g., a modified choline group). In certain embodiments, a phospholipid with a modified head is DSPC, or analog thereof, with a modified quaternary amine. For example, in embodiments of Formula (IV), at least one of R¹ is not methyl. In certain embodiments, at least one of R¹ is not hydrogen or methyl. In certain embodiments, the compound of Formula (IV) is of one of the following formulae:

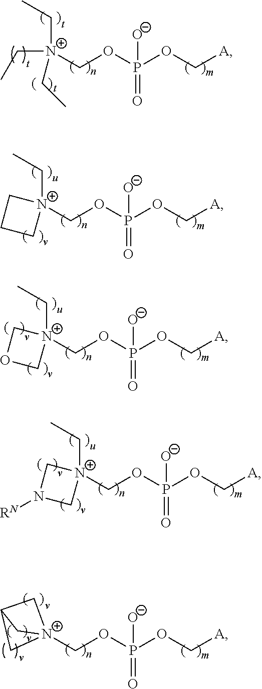

or a salt thereof, wherein:
each t is independently 1, 2, 3, 4, 5, 6, 7, 8, 9, or 10;
each u is independently 0, 1, 2, 3, 4, 5, 6, 7, 8, 9, or 10; and
each v is independently 1, 2, or 3.

In certain embodiments, a compound of Formula (IV) is of Formula (IV-a):

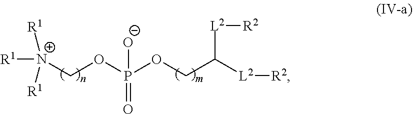

or a salt thereof.

In certain embodiments, a phospholipid useful or potentially useful in the present invention comprises a cyclic moiety in place of the glyceride moiety. In certain embodiments, a phospholipid useful in the present invention is DSPC, or analog thereof, with a cyclic moiety in place of the glyceride moiety. In certain embodiments, the compound of Formula (IV) is of Formula (IV-b):

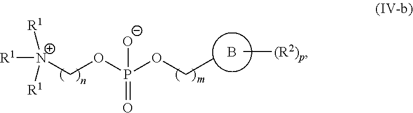

or a salt thereof.

### (ii) Phospholipid Tail Modifications

In certain embodiments, a phospholipid useful or potentially useful in the present invention comprises a modified tail. In certain embodiments, a phospholipid useful or potentially useful in the present invention is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (IV) is of Formula (IV-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), —NR^{N}C(═NR^{N})N(R^{N}), C(S), C(S)N(R^{N}), NR^{N}C(S), NR^{N}C(S)N(R^{N}), S(O), OS(O), S(O)O, OS(O)O, —OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or N(R^{N})S(O)₂O.

In certain embodiments, the compound of Formula (IV) is of Formula (IV-c):

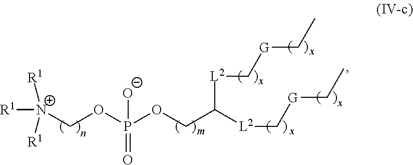

or a salt thereof, wherein:
each x is independently an integer between 0-30, inclusive; and
each instance is G is independently selected from the group consisting of optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), —C(O)O, OC(O), OC(O)O, OC(O)N(R^{N}), NR^{N}C(O)O, C(O)S, SC(O), C(═NR^{N}), C(═NR^{N})N(R^{N}), —NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), —S(O)O, OS(O)O, OS(O)₂, S(O)₂O, OS(O)₂O, N(R^{N})S(O), S(O)N(R^{N}), N(R^{N})S(O)N(R^{N}), —OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂₀. Each possibility represents a separate embodiment of the present invention.

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful or potentially useful in the present invention is a compound of Formula (IV), wherein n is 1, 3, 4, 5, 6, 7, 8, 9, or 10. For example, in certain embodiments, a compound of Formula (IV) is of one of the following formulae:

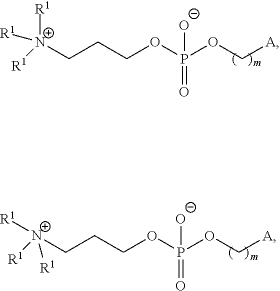

or a salt thereof.

### Alternative Lipids

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful.

In certain embodiments, an alternative lipid is used in place of a phospholipid of the present disclosure.

In certain embodiments, an alternative lipid of the invention is oleic acid.

In certain embodiments, the alternative lipid is one of the following:

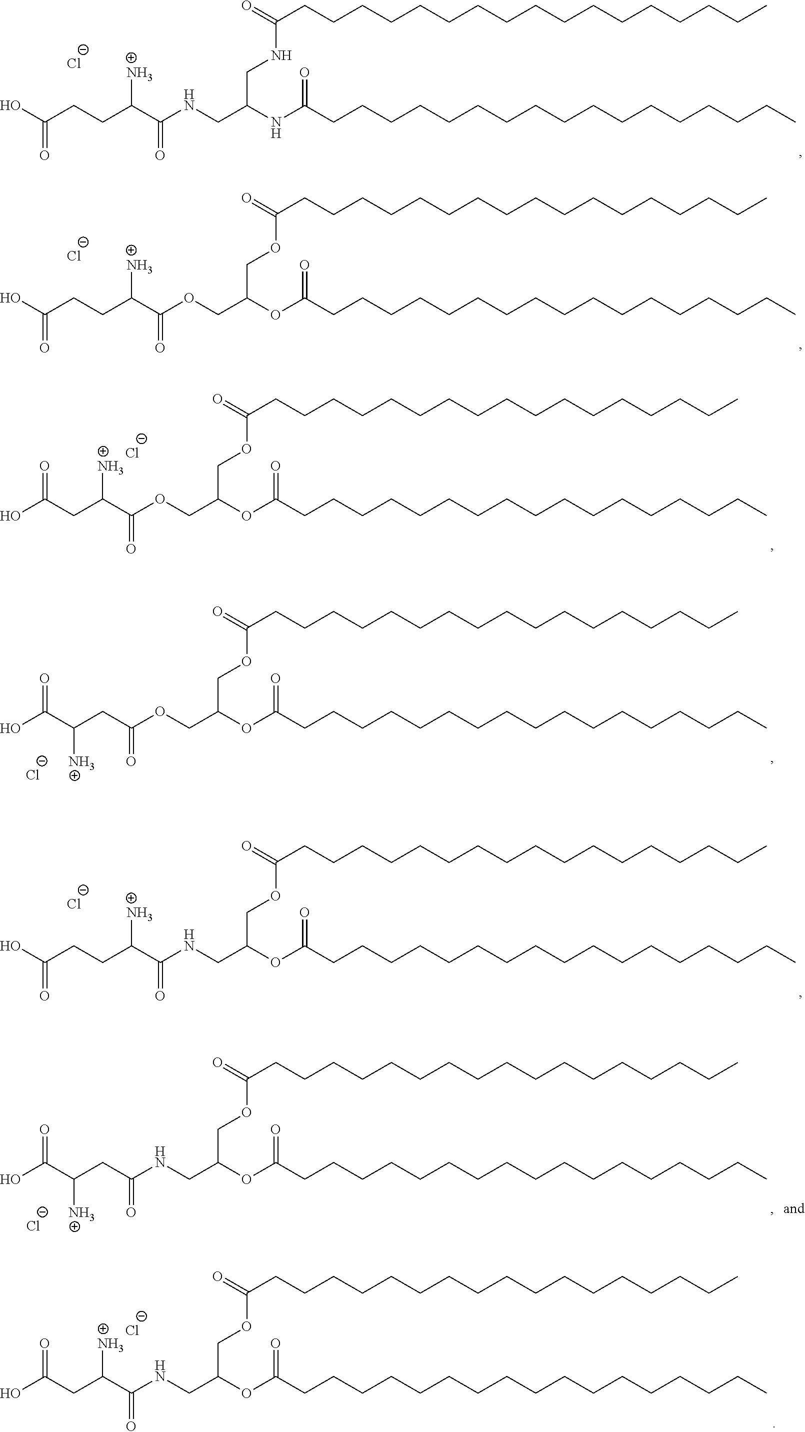

### Structural Lipids

The lipid composition of a pharmaceutical composition disclosed herein can comprise one or more structural lipids. As used herein, the term “structural lipid” refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. Application No. 62/520,530.

### Polyethylene Glycol (PEG)-Lipids

The lipid composition of a pharmaceutical composition disclosed herein can comprise one or more a polyethylene glycol (PEG) lipid.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In one embodiment, the PEG-lipid is PEG₂ₖ-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

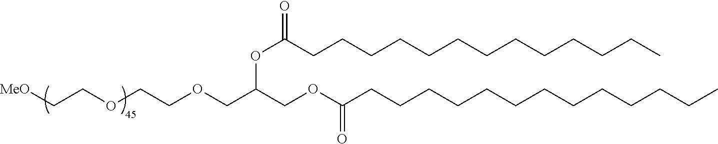

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In certain embodiments, a PEG lipid useful in the present invention is a compound of Formula (V). Provided herein are compounds of Formula (V):

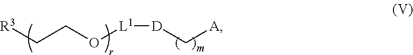

or salts thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, OC(O), OC(O)O, OC(O)N(R^{N}), —NR^{N}C(O)O, or NR^{N}C(O)N(R^{N});
D is a moiety obtained by click chemistry or a moiety cleavable under physiological conditions;
m is 0, 1, 2, 3, 4, 5, 6, 7, 8, 9, or 10;
A is of the formula:

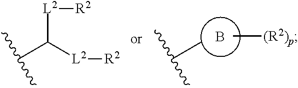

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or —NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, —C(O), C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), —NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Fomula (V) is a PEG-OH lipid (i.e., R³ is —OR^{O}, and R^{O} is hydrogen). In certain embodiments, the compound of Formula (V) is of Formula (V-OH):

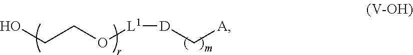

or a salt thereof.

In certain embodiments, a PEG lipid useful in the present invention is a PEGylated fatty acid. In certain embodiments, a PEG lipid useful in the present invention is a compound of Formula (VI). Provided herein are compounds of Formula (VI):

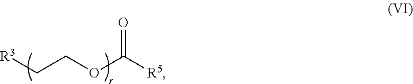

or a salts thereof, wherein:
R³ is-OR^{O};
R^{O} is hydrogen, optionally substituted alkyl or an oxygen protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), —NR^{N}C(O)N(R^{N}), C(O)O, OC(O), OC(O)O, OC(O)N(R^{N}), NR^{N}C(O)O, C(O)S, SC(O), C(═NR^{N}), —C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O; and
each instance of R^{N} is independently hydrogen, optionally substituted alkyl, or a nitrogen protecting group.

In certain embodiments, the compound of Formula (VI) is of Formula (VI-OH):

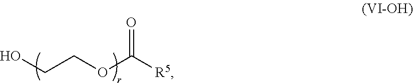

or a salt thereof. In some embodiments, r is 45.

In yet other embodiments the compound of Formula (VI) is:

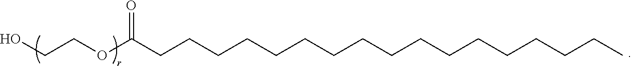

or a salt thereof.

In one embodiment, the compound of Formula (VI) is

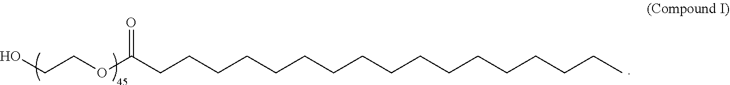

In some aspects, the lipid composition of the pharmaceutical compositions disclosed herein does not comprise a PEG-lipid.

In some embodiments, the PEG-lipids may be one or more of the PEG lipids described in U.S. Application No. 62/520,530.

In some embodiments, a PEG lipid of the invention comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, a LNP of the invention comprises an ionizable cationic lipid of any of Formula I, II or III, a phospholipid comprising DSPC, a structural lipid, and a PEG lipid comprising PEG-DMG.

In some embodiments, a LNP of the invention comprises an ionizable cationic lipid of any of Formula I, II or III, a phospholipid comprising DSPC, a structural lipid, and a PEG lipid comprising a compound having Formula VI.

In some embodiments, a LNP of the invention comprises an ionizable cationic lipid of Formula I, II or III, a phospholipid comprising a compound having Formula IV, a structural lipid, and the PEG lipid comprising a compound having Formula V or VI.

In some embodiments, a LNP of the invention comprises an ionizable cationic lipid of Formula I, II or III, a phospholipid comprising a compound having Formula IV, a structural lipid, and the PEG lipid comprising a compound having Formula V or VI.

In some embodiments, a LNP of the invention comprises an ionizable cationic lipid of Formula I, II or III, a phospholipid having Formula IV, a structural lipid, and a PEG lipid comprising a compound having Formula VI.

In some embodiments, a LNP of the invention comprises an ionizable cationic lipid of

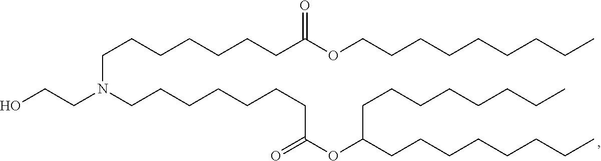 [SMILES: CCCCCCCCCOC(=O)CCCCCCCN(CCO)CCCCCCCC(=O)OC(CCCCCCCC)CCCCCCCC]

and a PEG lipid comprising Formula VI.

In some embodiments, a LNP of the invention comprises an ionizable cationic lipid of

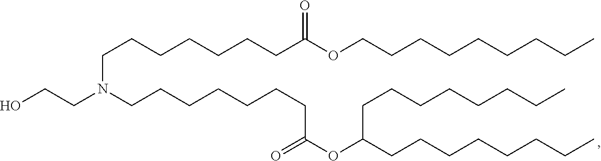 [SMILES: CCCCCCCCCOC(=O)CCCCCCCN(CCO)CCCCCCCC(=O)OC(CCCCCCCC)CCCCCCCC]

and an alternative lipid comprising oleic acid.

In some embodiments, a LNP of the invention comprises an ionizable cationic lipid of

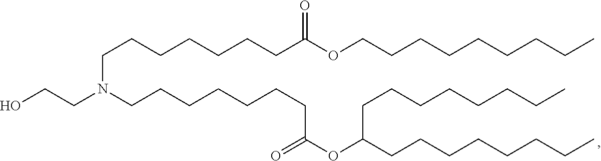 [SMILES: CCCCCCCCCOC(=O)CCCCCCCN(CCO)CCCCCCCC(=O)OC(CCCCCCCC)CCCCCCCC]

an alternative lipid comprising oleic acid, a structural lipid comprising cholesterol, and a PEG lipid comprising a compound having Formula VI.

In some embodiments, a LNP of the invention comprises an ionizable cationic lipid of

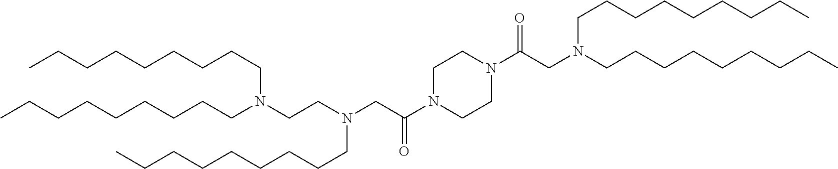 [SMILES: CCCCCCCCCN(CCCCCCCCC)CCN(CCCCCCCCC)CC(=O)N1CCN(C(=O)CN(CCCCCCCCC)CCCCCCCCC)CC1]

a phospholipid comprising DOPE, a structural lipid comprising cholesterol, and a PEG lipid comprising a compound having Formula VI.

In some embodiments, a LNP of the invention comprises an ionizable cationic lipid of
a phospholipid comprising DOPE, a structural lipid comprising cholesterol, and a PEG lipid comprising a compound having Formula VII.

In some embodiments, a LNP of the invention comprises an N:P ratio of from about 2:1 to about 30:1.

In some embodiments, a LNP of the invention comprises an N:P ratio of about 6:1.

In some embodiments, a LNP of the invention comprises an N:P ratio of about 3:1.

In some embodiments, a LNP of the invention comprises a wt/wt ratio of the ionizable cationic lipid component to the RNA of from about 10:1 to about 100:1.

In some embodiments, a LNP of the invention comprises a wt/wt ratio of the ionizable cationic lipid component to the RNA of about 20:1.

In some embodiments, a LNP of the invention comprises a wt/wt ratio of the ionizable cationic lipid component to the RNA of about 10:1.

In some embodiments, a LNP of the invention has a mean diameter from about 50 nm to about 150 nm.

In some embodiments, a LNP of the invention has a mean diameter from about 70 nm to about 120 nm.

As used herein, the term “alkyl”, “alkyl group”, or “alkylene” means a linear or branched, saturated hydrocarbon including one or more carbon atoms (e.g., one, two, three, four, five, six, seven, eight, nine, ten, eleven, twelve, thirteen, fourteen, fifteen, sixteen, seventeen, eighteen, nineteen, twenty, or more carbon atoms), which is optionally substituted. The notation “C₁₋₁₄ alkyl” means an optionally substituted linear or branched, saturated hydrocarbon including 1 14 carbon atoms. Unless otherwise specified, an alkyl group described herein refers to both unsubstituted and substituted alkyl groups.

As used herein, the term “alkenyl”, “alkenyl group”, or “alkenylene” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one double bond, which is optionally substituted. The notation “C₂-14 alkenyl” means an optionally substituted linear or branched hydrocarbon including 2 14 carbon atoms and at least one carbon-carbon double bond. An alkenyl group may include one, two, three, four, or more carbon-carbon double bonds. For example, C18 alkenyl may include one or more double bonds. A C18 alkenyl group including two double bonds may be a linoleyl group. Unless otherwise specified, an alkenyl group described herein refers to both unsubstituted and substituted alkenyl groups.

As used herein, the term “alkynyl”, “alkynyl group”, or “alkynylene” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one carbon-carbon triple bond, which is optionally substituted. The notation “C2-14 alkynyl” means an optionally substituted linear or branched hydrocarbon including 2 14 carbon atoms and at least one carbon-carbon triple bond. An alkynyl group may include one, two, three, four, or more carbon-carbon triple bonds. For example, C18 alkynyl may include one or more carbon-carbon triple bonds. Unless otherwise specified, an alkynyl group described herein refers to both unsubstituted and substituted alkynyl groups.

As used herein, the term “carbocycle” or “carbocyclic group” means an optionally substituted mono- or multi-cyclic system including one or more rings of carbon atoms. Rings may be three, four, five, six, seven, eight, nine, ten, eleven, twelve, thirteen, fourteen, fifteen, sixteen, seventeen, eighteen, nineteen, or twenty membered rings. The notation “C₃₋₆ carbocycle” means a carbocycle including a single ring having 3-6 carbon atoms. Carbocycles may include one or more carbon-carbon double or triple bonds and may be non-aromatic or aromatic (e.g., cycloalkyl or aryl groups). Examples of carbocycles include cyclopropyl, cyclopentyl, cyclohexyl, phenyl, naphthyl, and 1,2 dihydronaphthyl groups. The term “cycloalkyl” as used herein means a non-aromatic carbocycle and may or may not include any double or triple bond. Unless otherwise specified, carbocycles described herein refers to both unsubstituted and substituted carbocycle groups, i.e., optionally substituted carbocycles.

As used herein, the term “heterocycle” or “heterocyclic group” means an optionally substituted mono- or multi-cyclic system including one or more rings, where at least one ring includes at least one heteroatom. Heteroatoms may be, for example, nitrogen, oxygen, or sulfur atoms. Rings may be three, four, five, six, seven, eight, nine, ten, eleven, twelve, thirteen, or fourteen membered rings. Heterocycles may include one or more double or triple bonds and may be non-aromatic or aromatic (e.g., heterocycloalkyl or heteroaryl groups). Examples of heterocycles include imidazolyl, imidazolidinyl, oxazolyl, oxazolidinyl, thiazolyl, thiazolidinyl, pyrazolidinyl, pyrazolyl, isoxazolidinyl, isoxazolyl, isothiazolidinyl, isothiazolyl, morpholinyl, pyrrolyl, pyrrolidinyl, furyl, tetrahydrofuryl, thiophenyl, pyridinyl, piperidinyl, quinolyl, and isoquinolyl groups. The term “heterocycloalkyl” as used herein means a non-aromatic heterocycle and may or may not include any double or triple bond. Unless otherwise specified, heterocycles described herein refers to both unsubstituted and substituted heterocycle groups, i.e., optionally substituted heterocycles.

As used herein, the term “heteroalkyl”, “heteroalkenyl”, or “heteroalkynyl”, refers respectively to an alkyl, alkenyl, alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. Unless otherwise specified, heteroalkyls, heteroalkenyls, or heteroalkynyls described herein refers to both unsubstituted and substituted heteroalkyls, heteroalkenyls, or heteroalkynyls, i.e., optionally substituted heteroalkyls, heteroalkenyls, or heteroalkynyls.

As used herein, a “biodegradable group” is a group that may facilitate faster metabolism of a lipid in a mammalian entity. A biodegradable group may be selected from the group consisting of, but is not limited to, —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group. As used herein, an “aryl group” is an optionally substituted carbocyclic group including one or more aromatic rings. Examples of aryl groups include phenyl and naphthyl groups. As used herein, a “heteroaryl group” is an optionally substituted heterocyclic group including one or more aromatic rings. Examples of heteroaryl groups include pyrrolyl, furyl, thiophenyl, imidazolyl, oxazolyl, and thiazolyl. Both aryl and heteroaryl groups may be optionally substituted. For example, M and M′ can be selected from the non-limiting group consisting of optionally substituted phenyl, oxazole, and thiazole. In the formulas herein, M and M′ can be independently selected from the list of biodegradable groups above. Unless otherwise specified, aryl or heteroaryl groups described herein refers to both unsubstituted and substituted groups, i.e., optionally substituted aryl or heteroaryl groups.

Alkyl, alkenyl, and cyclyl (e.g., carbocyclyl and heterocyclyl) groups may be optionally substituted unless otherwise specified. Optional substituents may be selected from the group consisting of, but are not limited to, a halogen atom (e.g., a chloride, bromide, fluoride, or iodide group), a carboxylic acid (e.g., C(O)OH), an alcohol (e.g., a hydroxyl, OH), an ester (e.g., C(O)OR OC(O)R), an aldehyde (e.g., C(O)H), a carbonyl (e.g., C(O)R, alternatively represented by C═O), an acyl halide (e.g., C(O)X, in which X is a halide selected from bromide, fluoride, chloride, and iodide), a carbonate (e.g., OC(O)OR), an alkoxy (e.g., OR), an acetal (e.g., C(OR)₂R″″, in which each OR are alkoxy groups that can be the same or different and R″″ is an alkyl or alkenyl group), a phosphate (e.g., P(O)₄₃—), a thiol (e.g., SH), a sulfoxide (e.g., S(O)R), a sulfinic acid (e.g., S(O)OH), a sulfonic acid (e.g., S(O)₂OH), a thial (e.g., C(S)H), a sulfate (e.g., S(O)₄₂—), a sulfonyl (e.g., S(O)₂), an amide (e.g., C(O)NR2, or N(R)C(O)R), an azido (e.g., N3), a nitro (e.g., NO2), a cyano (e.g., CN), an isocyano (e.g., NC), an acyloxy (e.g., OC(O)R), an amino (e.g., NR2, NRH, or NH2), a carbamoyl (e.g., OC(O)NR2, OC(O)NRH, or OC(O)NH2), a sulfonamide (e.g., S(O)₂NR2, S(O)₂NRH, S(O)₂NH2, N(R)S(O)₂R, N(H)S(O)₂R, N(R)S(O)₂H, or N(H)S(O)₂H), an alkyl group, an alkenyl group, and a cyclyl (e.g., carbocyclyl or heterocyclyl) group. In any of the preceding, R is an alkyl or alkenyl group, as defined herein. In some embodiments, the substituent groups themselves may be further substituted with, for example, one, two, three, four, five, or six substituents as defined herein. For example, a C1 6 alkyl group may be further substituted with one, two, three, four, five, or six substituents as described herein.

Compounds of the disclosure that contain nitrogens can be converted to N-oxides by treatment with an oxidizing agent (e.g., 3-chloroperoxybenzoic acid (mCPBA) and/or hydrogen peroxides) to afford other compounds of the disclosure. Thus, all shown and claimed nitrogen-containing compounds are considered, when allowed by valency and structure, to include both the compound as shown and its N-oxide derivative (which can be designated as N□O or N+—O—). Furthermore, in other instances, the nitrogens in the compounds of the disclosure can be converted to N-hydroxy or N-alkoxy compounds. For example, N-hydroxy compounds can be prepared by oxidation of the parent amine by an oxidizing agent such as m CPBA. All shown and claimed nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C₁-C6 alkyl, C₁-C₆ alkenyl, C₁-C₆ alkynyl, 3-14-membered carbocycle or 3-14-membered heterocycle) derivatives.

### Other Lipid Composition Components

The lipid composition of a pharmaceutical composition disclosed herein can include one or more components in addition to those described above. For example, the lipid composition can include one or more permeability enhancer molecules, carbohydrates, polymers, surface altering agents (e.g., surfactants), or other components. For example, a permeability enhancer molecule can be a molecule described by U.S. Patent Application Publication No. 2005/0222064. Carbohydrates can include simple sugars (e.g., glucose) and polysaccharides (e.g., glycogen and derivatives and analogs thereof).

A polymer can be included in and/or used to encapsulate or partially encapsulate a pharmaceutical composition disclosed herein (e.g., a pharmaceutical composition in lipid nanoparticle form). A polymer can be biodegradable and/or biocompatible. A polymer can be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates.

The ratio between the lipid composition and the polynucleotide range can be from about 10:1 to about 60:1 (wt/wt).

In some embodiments, the ratio between the lipid composition and the polynucleotide can be about 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1 or 60:1 (wt/wt). In some embodiments, the wt/wt ratio of the lipid composition to the polynucleotide encoding a therapeutic agent is about 20:1 or about 15:1.

In some embodiments, the pharmaceutical composition disclosed herein can contain more than one polypeptides. For example, a pharmaceutical composition disclosed herein can contain two or more polynucleotides (e.g., RNA, e.g., mRNA).

In one embodiment, the lipid nanoparticles described herein can comprise polynucleotides (e.g., mRNA) in a lipid:polynucleotide weight ratio of 5:1, 10:1, 15:1, 20:1, 25:1, 30:1, 35:1, 40:1, 45:1, 50:1, 55:1, 60:1 or 70:1, or a range or any of these ratios such as, but not limited to, 5:1 to about 10:1, from about 5:1 to about 15:1, from about 5:1 to about 20:1, from about 5:1 to about 25:1, from about 5:1 to about 30:1, from about 5:1 to about 35:1, from about 5:1 to about 40:1, from about 5:1 to about 45:1, from about 5:1 to about 50:1, from about 5:1 to about 55:1, from about 5:1 to about 60:1, from about 5:1 to about 70:1, from about 10:1 to about 15:1, from about 10:1 to about 20:1, from about 10:1 to about 25:1, from about 10:1 to about 30:1, from about 10:1 to about 35:1, from about 10:1 to about 40:1, from about 10:1 to about 45:1, from about 10:1 to about 50:1, from about 10:1 to about 55:1, from about 10:1 to about 60:1, from about 10:1 to about 70:1, from about 15:1 to about 20:1, from about 15:1 to about 25:1, from about 15:1 to about 30:1, from about 15:1 to about 35:1, from about 15:1 to about 40:1, from about 15:1 to about 45:1, from about 15:1 to about 50:1, from about 15:1 to about 55:1, from about 15:1 to about 60:1 or from about 15:1 to about 70:1.

In one embodiment, the lipid nanoparticles described herein can comprise the polynucleotide in a concentration from approximately 0.1 mg/ml to 2 mg/ml such as, but not limited to, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml or greater than 2.0 mg/ml.

### Nanoparticle Compositions

In some embodiments, the pharmaceutical compositions disclosed herein are formulated as lipid nanoparticles (LNP). Accordingly, the present disclosure also provides nanoparticle compositions comprising (i) a lipid composition comprising a delivery agent such as compound as described herein, and (ii) at least one mRNA encoding a polypeptide. In such nanoparticle composition, the lipid composition disclosed herein can encapsulate the at least one mRNA encoding a polypeptide.

Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Nanoparticle compositions include, for example, lipid nanoparticles (LNPs), liposomes, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In one embodiment, a lipid nanoparticle comprises an ionizable lipid, a structural lipid, a phospholipid, and mRNA. In some embodiments, the LNP comprises an ionizable lipid, a PEG-modified lipid, a sterol and a structural lipid. In some embodiments, the LNP has a molar ratio of about 20-60% ionizable lipid: about 5-25% structural lipid: about 25-55% sterol; and about 0.5-15% PEG-modified lipid.

In some embodiments, the LNP has a polydispersity value of less than 0.4. In some embodiments, the LNP has a net neutral charge at a neutral pH. In some embodiments, the LNP has a mean diameter of 50-150 nm. In some embodiments, the LNP has a mean diameter of 80-100 nm.

As generally defined herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, and polyketides, and prenol lipids. In some instances, the amphiphilic properties of some lipids leads them to form liposomes, vesicles, or membranes in aqueous media.

In some embodiments, a lipid nanoparticle (LNP) may comprise an ionizable lipid. As used herein, the term “ionizable lipid” has its ordinary meaning in the art and may refer to a lipid comprising one or more charged moieties. In some embodiments, an ionizable lipid may be positively charged or negatively charged. An ionizable lipid may be positively charged, in which case it can be referred to as “cationic lipid”. In certain embodiments, an ionizable lipid molecule may comprise an amine group, and can be referred to as an ionizable amino lipid. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired.

It should be understood that the terms “charged” or “charged moiety” does not refer to a “partial negative charge” or “partial positive charge” on a molecule. The terms “partial negative charge” and “partial positive charge” are given its ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

In some embodiments, the ionizable lipid is an ionizable amino lipid, sometimes referred to in the art as an “ionizable cationic lipid”. In one embodiment, the ionizable amino lipid may have a positively charged hydrophilic head and a hydrophobic tail that are connected via a linker structure.

In addition to these, an ionizable lipid may also be a lipid including a cyclic amine group.

In one embodiment, the ionizable lipid may be selected from, but not limited to, a ionizable lipid described in International Publication Nos. WO2013086354 and WO2013116126; the contents of each of which are herein incorporated by reference in their entirety.

In yet another embodiment, the ionizable lipid may be selected from, but not limited to, formula CLI-CLXXXXII of U.S. Pat. No. 7,404,969; each of which is herein incorporated by reference in their entirety.

In one embodiment, the lipid may be a cleavable lipid such as those described in International Publication No. WO2012170889, herein incorporated by reference in its entirety. In one embodiment, the lipid may be synthesized by methods known in the art and/or as described in International Publication Nos. WO2013086354; the contents of each of which are herein incorporated by reference in their entirety.

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential.

The size of the nanoparticles can help counter biological reactions such as, but not limited to, inflammation, or can increase the biological effect of the polynucleotide.

As used herein, “size” or “mean size” in the context of nanoparticle compositions refers to the mean diameter of a nanoparticle composition.

In one embodiment, the polynucleotide encoding a polypeptide is formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm, about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In one embodiment, the nanoparticles have a diameter from about 10 to 500 nm. In one embodiment, the nanoparticle has a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the largest dimension of a nanoparticle composition is 1 μm or shorter (e.g., 1 μm, 900 nm, 800 nm, 700 nm, 600 nm, 500 nm, 400 nm, 300 nm, 200 nm, 175 nm, 150 nm, 125 nm, 100 nm, 75 nm, 50 nm, or shorter).

A nanoparticle composition can be relatively homogenous. A polydispersity index can be used to indicate the homogeneity of a nanoparticle composition, e.g., the particle size distribution of the nanoparticle composition. A small (e.g., less than 0.3) polydispersity index generally indicates a narrow particle size distribution. A nanoparticle composition can have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition disclosed herein can be from about 0.10 to about 0.20.

The zeta potential of a nanoparticle composition can be used to indicate the electrokinetic potential of the composition. For example, the zeta potential can describe the surface charge of a nanoparticle composition. Nanoparticle compositions with relatively low charges, positive or negative, are generally desirable, as more highly charged species can interact undesirably with cells, tissues, and other elements in the body. In some embodiments, the zeta potential of a nanoparticle composition disclosed herein can be from about −10 mV to about +20 mV, from about −10 mV to about +15 mV, from about 10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

In some embodiments, the zeta potential of the lipid nanoparticles can be from about 0 mV to about 100 mV, from about 0 mV to about 90 mV, from about 0 mV to about 80 mV, from about 0 mV to about 70 mV, from about 0 mV to about 60 mV, from about 0 mV to about 50 mV, from about 0 mV to about 40 mV, from about 0 mV to about 30 mV, from about 0 mV to about 20 mV, from about 0 mV to about 10 mV, from about 10 mV to about 100 mV, from about 10 mV to about 90 mV, from about 10 mV to about 80 mV, from about 10 mV to about 70 mV, from about 10 mV to about 60 mV, from about 10 mV to about 50 mV, from about 10 mV to about 40 mV, from about 10 mV to about 30 mV, from about 10 mV to about 20 mV, from about 20 mV to about 100 mV, from about 20 mV to about 90 mV, from about 20 mV to about 80 mV, from about 20 mV to about 70 mV, from about 20 mV to about 60 mV, from about 20 mV to about 50 mV, from about 20 mV to about 40 mV, from about 20 mV to about 30 mV, from about 30 mV to about 100 mV, from about 30 mV to about 90 mV, from about 30 mV to about 80 mV, from about 30 mV to about 70 mV, from about 30 mV to about 60 mV, from about 30 mV to about 50 mV, from about 30 mV to about 40 mV, from about 40 mV to about 100 mV, from about 40 mV to about 90 mV, from about 40 mV to about 80 mV, from about 40 mV to about 70 mV, from about 40 mV to about 60 mV, and from about 40 mV to about 50 mV. In some embodiments, the zeta potential of the lipid nanoparticles can be from about 10 mV to about 50 mV, from about 15 mV to about 45 mV, from about 20 mV to about 40 mV, and from about 25 mV to about 35 mV. In some embodiments, the zeta potential of the lipid nanoparticles can be about 10 mV, about 20 mV, about 30 mV, about 40 mV, about 50 mV, about 60 mV, about 70 mV, about 80 mV, about 90 mV, and about 100 mV.

The term “encapsulation efficiency” of a polynucleotide describes the amount of the polynucleotide that is encapsulated by or otherwise associated with a nanoparticle composition after preparation, relative to the initial amount provided. As used herein, “encapsulation” can refer to complete, substantial, or partial enclosure, confinement, surrounding, or encasement.

Encapsulation efficiency is desirably high (e.g., close to 100%). The encapsulation efficiency can be measured, for example, by comparing the amount of the polynucleotide in a solution containing the nanoparticle composition before and after breaking up the nanoparticle composition with one or more organic solvents or detergents.

Fluorescence can be used to measure the amount of free polynucleotide in a solution. For the nanoparticle compositions described herein, the encapsulation efficiency of a polynucleotide can be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency can be at least 80%. In certain embodiments, the encapsulation efficiency can be at least 90%.

The amount of a polynucleotide present in a pharmaceutical composition disclosed herein can depend on multiple factors such as the size of the polynucleotide, desired target and/or application, or other properties of the nanoparticle composition as well as on the properties of the polynucleotide.

For example, the amount of an mRNA useful in a nanoparticle composition can depend on the size (expressed as length, or molecular mass), sequence, and other characteristics of the mRNA. The relative amounts of a polynucleotide in a nanoparticle composition can also vary.

The relative amounts of the lipid composition and the polynucleotide present in a lipid nanoparticle composition of the present disclosure can be optimized according to considerations of efficacy and tolerability. For compositions including an mRNA as a polynucleotide, the N:P ratio can serve as a useful metric.

As the N:P ratio of a nanoparticle composition controls both expression and tolerability, nanoparticle compositions with low N:P ratios and strong expression are desirable. N:P ratios vary according to the ratio of lipids to RNA in a nanoparticle composition.

In general, a lower N:P ratio is preferred. The one or more RNA, lipids, and amounts thereof can be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio can be from about 2:1 to about 8:1. In other embodiments, the N:P ratio is from about 5:1 to about 8:1. In certain embodiments, the N:P ratio is between 5:1 and 6:1. In one specific aspect, the N:P ratio is about is about 5.67:1.

In addition to providing nanoparticle compositions, the present disclosure also provides methods of producing lipid nanoparticles comprising encapsulating a polynucleotide. Such method comprises using any of the pharmaceutical compositions disclosed herein and producing lipid nanoparticles in accordance with methods of production of lipid nanoparticles known in the art. See, e.g., Wang et al. (2015) “Delivery of oligonucleotides with lipid nanoparticles” Adv. Drug Deliv. Rev. 87:68-80; Silva et al. (2015) “Delivery Systems for Biopharmaceuticals. Part I: Nanoparticles and Microparticles” Curr. Pharm. Technol. 16: 940-954; Naseri et al. (2015) “Solid Lipid Nanoparticles and Nanostructured Lipid Carriers: Structure, Preparation and Application” Adv. Pharm. Bull. 5:305-13; Silva et al. (2015) “Lipid nanoparticles for the delivery of biopharmaceuticals” Curr. Pharm. Biotechnol. 16:291-302, and references cited therein.

### Other Delivery Agents

### Liposomes, Lipoplexes, and Lipid Nanoparticles

In some embodiments, the compositions or formulations of the present disclosure comprise a delivery agent, e.g., a liposome, a lioplexes, a lipid nanoparticle, or any combination thereof. The polynucleotides described herein (e.g., a polynucleotide comprising a nucleotide sequence encoding a polypeptide) can be formulated using one or more liposomes, lipoplexes, or lipid nanoparticles. Liposomes, lipoplexes, or lipid nanoparticles can be used to improve the efficacy of the mRNAs directed protein production as these formulations can increase cell transfection by the mRNA; and/or increase the translation of encoded protein. The liposomes, lipoplexes, or lipid nanoparticles can also be used to increase the stability of the mRNAs.

Liposomes are artificially-prepared vesicles that can primarily be composed of a lipid bilayer and can be used as a delivery vehicle for the administration of pharmaceutical formulations. Liposomes can be of different sizes. A multilamellar vesicle (MLV) can be hundreds of nanometers in diameter, and can contain a series of concentric bilayers separated by narrow aqueous compartments. A small unicellular vesicle (SUV) can be smaller than 50 nm in diameter, and a large unilamellar vesicle (LUV) can be between 50 and 500 nm in diameter. Liposome design can include, but is not limited to, opsonins or ligands to improve the attachment of liposomes to unhealthy tissue or to activate events such as, but not limited to, endocytosis. Liposomes can contain a low or a high pH value in order to improve the delivery of the pharmaceutical formulations.

The formation of liposomes can depend on the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimal size, polydispersity and the shelf-life of the vesicles for the intended application, and the batch-to-batch reproducibility and scale up production of safe and efficient liposomal products, etc.

As a non-limiting example, liposomes such as synthetic membrane vesicles can be prepared by the methods, apparatus and devices described in U.S. Pub. Nos. US20130177638, US20130177637, US20130177636, US20130177635, US20130177634, US20130177633, US20130183375, US20130183373, and US20130183372. In some embodiments, the mRNAs described herein can be encapsulated by the liposome and/or it can be contained in an aqueous core that can then be encapsulated by the liposome as described in, e.g., Intl. Pub. Nos. WO2012031046, WO2012031043, WO2012030901, WO2012006378, and WO2013086526; and U.S. Pub. Nos. US20130189351, US20130195969 and US20130202684. Each of the references in herein incorporated by reference in its entirety.

In some embodiments, the mRNAs described herein can be formulated in a cationic oil-in-water emulsion where the emulsion particle comprises an oil core and a cationic lipid that can interact with the mRNA anchoring the molecule to the emulsion particle. In some embodiments, the mRNAs described herein can be formulated in a water-in-oil emulsion comprising a continuous hydrophobic phase in which the hydrophilic phase is dispersed. Exemplary emulsions can be made by the methods described in Intl. Pub. Nos. WO2012006380 and WO201087791, each of which is herein incorporated by reference in its entirety.

In some embodiments, the mRNAs described herein can be formulated in a lipid-polycation complex. The formation of the lipid-polycation complex can be accomplished by methods as described in, e.g., U.S. Pub. No. US20120178702. As a non-limiting example, the polycation can include a cationic peptide or a polypeptide such as, but not limited to, polylysine, polyornithine and/or polyarginine and the cationic peptides described in Intl. Pub. No. WO2012013326 or U.S. Pub. No. US20130142818. Each of the references is herein incorporated by reference in its entirety.

In some embodiments, the mRNAs described herein can be formulated in a lipid nanoparticle (LNP) such as those described in Intl. Pub. Nos. WO2013123523, WO2012170930, WO2011127255 and WO2008103276; and U.S. Pub. No. US20130171646, each of which is herein incorporated by reference in its entirety.

Lipid nanoparticle formulations typically comprise one or more lipids. In some embodiments, the lipid is an ionizable lipid (e.g., an ionizable amino lipid), sometimes referred to in the art as an “ionizable cationic lipid”. In some embodiments, lipid nanoparticle formulations further comprise other components, including a phospholipid, a structural lipid, and a molecule capable of reducing particle aggregation, for example a PEG or PEG-modified lipid.

Exemplary ionizable lipids include, but not limited to, any one of Compounds 1-342 disclosed herein, DLin-MC3-DMA (MC3), DLin-DMA, DLenDMA, DLin-D-DMA, DLin-K-DMA, DLin-M-C₂-DMA, DLin-K-DMA, DLin-KC2-DMA, DLin-KC3-DMA, DLin-KC4-DMA, DLin-C₂K-DMA, DLin-MP-DMA, DODMA, 98N12-5, C₁₂-200, DLin-C-DAP, DLin-DAC, DLinDAP, DLinAP, DLin-EG-DMA, DLin-2-DMAP, KL10, KL22, KL25, Octyl-CLinDMA, Octyl-CLinDMA (2R), Octyl-CLinDMA (2S), and any combination thereof. Other exemplary ionizable lipids include, (13Z,16Z)-N,N-dimethyl-3-nonyldocosa-13,16-dien-1-amine (L608), (20Z,23Z)-N,N-dimethylnonacosa-20,23-dien-10-amine, (17Z,20Z)-N,N-dimemylhexacosa-17,20-dien-9-amine, (16Z,19Z)-N5N-dimethylpentacosa-16,19-dien-8-amine, (13Z,16Z)-N,N-dimethyldocosa-13,16-dien-5-amine, (12Z,15Z)-N,N-dimethylhenicosa-12,15-dien-4-amine, (14Z,17Z)-N,N-dimethyltricosa-14,17-dien-6-amine, (15Z,18Z)-N,N-dimethyltetracosa-15,18-dien-7-amine, (18Z,21Z)-N,N-dimethylheptacosa-18,21-dien-10-amine, (15Z,18Z)-N,N-dimethyltetracosa-15,18-dien-5-amine, (14Z,17Z)-N,N-dimethyltricosa-14,17-dien-4-amine, (19Z,22Z)-N,N-dimeihyloctacosa-19,22-dien-9-amine, (18Z,21Z)-N,N-dimethylheptacosa-18,21-dien-8-amine, (17Z,20Z)-N,N-dimethylhexacosa-17,20-dien-7-amine, (16Z,19Z)-N,N-dimethylpentacosa-16,19-dien-6-amine, (22Z,25Z)-N,N-dimethylhentriaconta-22,25-dien-10-amine, (21Z,24Z)-N,N-dimethyltriaconta-21,24-dien-9-amine, (18Z)-N,N-dimetylheptacos-18-en-10-amine, (17Z)-N,N-dimethylhexacos-17-en-9-amine, (19Z,22Z)-N,N-dimethyloctacosa-19,22-dien-7-amine, N,N-dimethylheptacosan-10-amine, (20Z,23Z)-N-ethyl-N-methylnonacosa-20,23-dien-10-amine, 1-[(11Z,14Z)-1-nonylicosa-11,14-dien-1-yl]pyrrolidine, (20Z)-N,N-dimethylheptacos-20-en-10-amine, (15Z)-N,N-dimethyl eptacos-15-en-10-amine, (14Z)-N,N-dimethylnonacos-14-en-10-amine, (17Z)-N,N-dimethylnonacos-17-en-10-amine, (24Z)-N,N-dimethyltritriacont-24-en-10-amine, (20Z)-N,N-dimethylnonacos-20-en-10-amine, (22Z)-N,N-dimethylhentriacont-22-en-10-amine, (16Z)-N,N-dimethylpentacos-16-en-8-amine, (12Z,15Z)-N,N-dimethyl-2-nonylhenicosa-12,15-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{[(1R,2R)-2-pentylcyclopropyl]methyl}cyclopropyl]nonadecan-10-amine, N,N-dimethyl-1-[(1S,2R)-2-octylcyclopropyl]hexadecan-8-amine, N,N-dimethyl-[(1R,2S)-2-undecyIcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethylIpyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine, and (11E,20Z,23Z)-N,N-dimethylnonacosa-11,20,2-trien-10-amine, and any combination thereof.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin. In some embodiments, the phospholipids are DLPC, DMPC, DOPC, DPPC, DSPC, DUPC, 18:0 Diether PC, DLnPC, DAPC, DHAPC, DOPE, 4ME 16:0 PE, DSPE, DLPE, DLnPE, DAPE, DHAPE, DOPG, and any combination thereof. In some embodiments, the phospholipids are MPPC, MSPC, PMPC, PSPC, SMPC, SPPC, DHAPE, DOPG, and any combination thereof. In some embodiments, the amount of phospholipids (e.g., DSPC) in the lipid composition ranges from about 1 mol % to about 20 mol %.

The structural lipids include sterols and lipids containing sterol moieties. In some embodiments, the structural lipids include cholesterol, fecosterol, sitosterol, ergosterol, campesterol, stigmasterol, brassicasterol, tomatidine, tomatine, ursolic acid, alpha-tocopherol, and mixtures thereof. In some embodiments, the structural lipid is cholesterol. In some embodiments, the amount of the structural lipids (e.g., cholesterol) in the lipid composition ranges from about 20 mol % to about 60 mol %.

The PEG-modified lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG DMPE, PEG-DPPC, or a PEG-DSPE lipid. In some embodiments, the PEG-lipid are 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA). In some embodiments, the PEG moiety has a size of about 1000, 2000, 5000, 10,000, 15,000 or 20,000 daltons. In some embodiments, the amount of PEG-lipid in the lipid composition ranges from about 0 mol % to about 5 mol %.

In some embodiments, the LNP formulations described herein can additionally comprise a permeability enhancer molecule. Non-limiting permeability enhancer molecules are described in U.S. Pub. No. US20050222064, herein incorporated by reference in its entirety.

The LNP formulations can further contain a phosphate conjugate. The phosphate conjugate can increase in vivo circulation times and/or increase the targeted delivery of the nanoparticle. Phosphate conjugates can be made by the methods described in, e.g., Intl. Pub. No. WO2013033438 or U.S. Pub. No. US20130196948. The LNP formulation can also contain a polymer conjugate (e.g., a water soluble conjugate) as described in, e.g., U.S. Pub. Nos. US20130059360, US20130196948, and US20130072709. Each of the references is herein incorporated by reference in its entirety.

The LNP formulations can comprise a conjugate to enhance the delivery of nanoparticles of the present invention in a subject. Further, the conjugate can inhibit phagocytic clearance of the nanoparticles in a subject. In some embodiments, the conjugate can be a “self” peptide designed from the human membrane protein CD47 (e.g., the “self” particles described by Rodriguez et al, Science 2013 339, 971-975, herein incorporated by reference in its entirety). As shown by Rodriguez et al. the self peptides delayed macrophage-mediated clearance of nanoparticles which enhanced delivery of the nanoparticles.

The LNP formulations can comprise a carbohydrate carrier. As a non-limiting example, the carbohydrate carrier can include, but is not limited to, an anhydride-modified phytoglycogen or glycogen-type material, phytoglycogen octenyl succinate, phytoglycogen beta-dextrin, anhydride-modified phytoglycogen beta-dextrin (e.g., Intl. Pub. No. WO2012109121, herein incorporated by reference in its entirety).

The LNP formulations can be coated with a surfactant or polymer to improve the delivery of the particle. In some embodiments, the LNP can be coated with a hydrophilic coating such as, but not limited to, PEG coatings and/or coatings that have a neutral surface charge as described in U.S. Pub. No. US20130183244, herein incorporated by reference in its entirety.

The LNP formulations can be engineered to alter the surface properties of particles so that the lipid nanoparticles can penetrate the mucosal barrier as described in U.S. Pat. No. 8,241,670 or Intl. Pub. No. WO2013110028, each of which is herein incorporated by reference in its entirety.

The LNP engineered to penetrate mucus can comprise a polymeric material (i.e., a polymeric core) and/or a polymer-vitamin conjugate and/or a tri-block co-polymer. The polymeric material can include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates.

LNP engineered to penetrate mucus can also include surface altering agents such as, but not limited to, mRNAs, anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin β4 dornase alfa, neltenexine, erdosteine) and various DNases including rhDNase.

In some embodiments, the mucus penetrating LNP can be a hypotonic formulation comprising a mucosal penetration enhancing coating. The formulation can be hypotonic for the epithelium to which it is being delivered. Non-limiting examples of hypotonic formulations can be found in, e.g., Intl. Pub. No. WO2013110028, herein incorporated by reference in its entirety.

In some embodiments, the mRNA described herein is formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, Mass.), and polyethylenimine (PEI) or protamine-based targeted and non-targeted delivery of nucleic acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293 Weide et al. J Immunother. 2009 32:498-507; Weide et al. J Immunother. 2008 31:180-188; Pascolo Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci USA. 2007 6; 104:4095-4100; deFougerolles Hum Gene Ther. 2008 19:125-132; all of which are incorporated herein by reference in its entirety).

In some embodiments, the mRNAs described herein are formulated as a solid lipid nanoparticle (SLN), which can be spherical with an average diameter between 10 to 1000 nm. SLN possess a solid lipid core matrix that can solubilize lipophilic molecules and can be stabilized with surfactants and/or emulsifiers. Exemplary SLN can be those as described in Intl. Pub. No. WO2013105101, herein incorporated by reference in its entirety.

In some embodiments, the mRNAs described herein can be formulated for controlled release and/or targeted delivery. As used herein, “controlled release” refers to a pharmaceutical composition or compound release profile that conforms to a particular pattern of release to effect a therapeutic outcome. In one embodiment, the mRNAs can be encapsulated into a delivery agent described herein and/or known in the art for controlled release and/or targeted delivery. As used herein, the term “encapsulate” means to enclose, surround or encase. As it relates to the formulation of the compounds of the invention, encapsulation can be substantial, complete or partial. The term “substantially encapsulated” means that at least greater than 50, 60, 70, 80, 85, 90, 95, 96, 97, 98, 99, or greater than 99% of the pharmaceutical composition or compound of the invention can be enclosed, surrounded or encased within the delivery agent. “Partially encapsulation” means that less than 10, 10, 20, 30, 40 50 or less of the pharmaceutical composition or compound of the invention can be enclosed, surrounded or encased within the delivery agent.

Advantageously, encapsulation can be determined by measuring the escape or the activity of the pharmaceutical composition or compound of the invention using fluorescence and/or electron micrograph. For example, at least 1, 5, 10, 20, 30, 40, 50, 60, 70, 80, 85, 90, 95, 96, 97, 98, 99, 99.9, or greater than 99% of the pharmaceutical composition or compound of the invention are encapsulated in the delivery agent.

In some embodiments, the mRNAs described herein can be encapsulated in a therapeutic nanoparticle, referred to herein as “therapeutic nanoparticle mRNAs.” Therapeutic nanoparticles can be formulated by methods described in, e.g., Intl. Pub. Nos. WO2010005740, WO2010030763, WO2010005721, WO2010005723, and WO2012054923; and U.S. Pub. Nos. US20110262491, US20100104645, US20100087337, US20100068285, US20110274759, US20100068286, US20120288541, US20120140790, US20130123351 and US20130230567; and U.S. Pat. Nos. 8,206,747, 8,293,276, 8,318,208 and 8,318,211, each of which is herein incorporated by reference in its entirety.

In some embodiments, the therapeutic nanoparticle mRNA can be formulated for sustained release. As used herein, “sustained release” refers to a pharmaceutical composition or compound that conforms to a release rate over a specific period of time. The period of time can include, but is not limited to, hours, days, weeks, months and years. As a non-limiting example, the sustained release nanoparticle of the mRNAs described herein can be formulated as disclosed in Intl. Pub. No.

WO2010075072 and U.S. Pub. Nos. US20100216804, US20110217377, US20120201859 and US20130150295, each of which is herein incorporated by reference in their entirety.

In some embodiments, the therapeutic nanoparticle mRNA can be formulated to be target specific, such as those described in Intl. Pub. Nos. WO2008121949, WO2010005726, WO2010005725, WO2011084521 and WO2011084518; and U.S. Pub. Nos. US20100069426, US20120004293 and US20100104655, each of which is herein incorporated by reference in its entirety.

The LNPs can be prepared using microfluidic mixers or micromixers. Exemplary microfluidic mixers can include, but are not limited to, a slit interdigital micromixer including, but not limited to those manufactured by Microinnova (Allerheiligen bei Wildon, Austria) and/or a staggered herringbone micromixer (SHM) (see Zhigaltsev et al., “Bottom-up design and synthesis of limit size lipid nanoparticle systems with aqueous and triglyceride cores using millisecond microfluidic mixing,” Langmuir 28:3633-40 (2012); Belliveau et al., “Microfluidic synthesis of highly potent limit-size lipid nanoparticles for in vivo delivery of siRNA,” Molecular Therapy-Nucleic Acids. 1:e37 (2012); Chen et al., “Rapid discovery of potent siRNA-containing lipid nanoparticles enabled by controlled microfluidic formulation,” J. Am. Chem. Soc. 134(16):6948-51 (2012); each of which is herein incorporated by reference in its entirety). Exemplary micromixers include Slit Interdigital Microstructured Mixer (SIMM-V2) or a Standard Slit Interdigital Micro Mixer (SSIMM) or Caterpillar (CPMM) or Impinging-jet (IJMM) from the Institut für Mikrotechnik Mainz GmbH, Mainz Germany. In some embodiments, methods of making LNP using SHM further comprise mixing at least two input streams wherein mixing occurs by microstructure-induced chaotic advection (MICA). According to this method, fluid streams flow through channels present in a herringbone pattern causing rotational flow and folding the fluids around each other. This method can also comprise a surface for fluid mixing wherein the surface changes orientations during fluid cycling. Methods of generating LNPs using SHM include those disclosed in U.S. Pub. Nos. US20040262223 and US20120276209, each of which is incorporated herein by reference in their entirety.

In some embodiments, the mRNAs described herein can be formulated in lipid nanoparticles using microfluidic technology (see Whitesides, George M., “The Origins and the Future of Microfluidics,” Nature 442: 368-373 (2006); and Abraham et al., “Chaotic Mixer for Microchannels,” Science 295: 647-651 (2002); each of which is herein incorporated by reference in its entirety). In some embodiments, the mRNAs can be formulated in lipid nanoparticles using a micromixer chip such as, but not limited to, those from Harvard Apparatus (Holliston, Mass.) or Dolomite Microfluidics (Royston, UK). A micromixer chip can be used for rapid mixing of two or more fluid streams with a split and recombine mechanism.

In some embodiments, the mRNAs described herein can be formulated in lipid nanoparticles having a diameter from about 1 nm to about 100 nm such as, but not limited to, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In some embodiments, the lipid nanoparticles can have a diameter from about 10 to 500 nm. In one embodiment, the lipid nanoparticle can have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the mRNAs can be delivered using smaller LNPs. Such particles can comprise a diameter from below 0.1 μm up to 100 nm such as, but not limited to, less than 0.1 μm, less than 1.0 μm, less than 5 μm, less than 10 μm, less than 15 um, less than 20 um, less than 25 um, less than 30 um, less than 35 um, less than 40 um, less than 50 um, less than 55 um, less than 60 um, less than 65 um, less than 70 um, less than 75 um, less than 80 um, less than 85 um, less than 90 um, less than 95 um, less than 100 um, less than 125 um, less than 150 um, less than 175 um, less than 200 um, less than 225 um, less than 250 um, less than 275 um, less than 300 um, less than 325 um, less than 350 um, less than 375 um, less than 400 um, less than 425 um, less than 450 um, less than 475 um, less than 500 um, less than 525 um, less than 550 um, less than 575 um, less than 600 um, less than 625 um, less than 650 um, less than 675 um, less than 700 um, less than 725 um, less than 750 um, less than 775 um, less than 800 um, less than 825 um, less than 850 um, less than 875 um, less than 900 um, less than 925 um, less than 950 um, or less than 975 um.

The nanoparticles and microparticles described herein can be geometrically engineered to modulate macrophage and/or the immune response. The geometrically engineered particles can have varied shapes, sizes and/or surface charges to incorporate the mRNAs described herein for targeted delivery such as, but not limited to, pulmonary delivery (see, e.g., Intl. Pub. No. WO20130821 11, herein incorporated by reference in its entirety). Other physical features the geometrically engineering particles can include, but are not limited to, fenestrations, angled arms, asymmetry and surface roughness, charge that can alter the interactions with cells and tissues.

In some embodiment, the nanoparticles described herein are stealth nanoparticles or target-specific stealth nanoparticles such as, but not limited to, those described in U.S. Pub. No. US20130172406, herein incorporated by reference in its entirety. The stealth or target-specific stealth nanoparticles can comprise a polymeric matrix, which can comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polyesters, polyanhydrides, polyethers, polyurethanes, polymethacrylates, polyacrylates, polycyanoacrylates, or combinations thereof.

### Lipidoids

In some embodiments, the compositions or formulations of the present disclosure comprise a delivery agent, e.g., a lipidoid. The mRNAs described herein (e.g., an mRNA comprising a nucleotide sequence encoding a polypeptide) can be formulated with lipidoids. Complexes, micelles, liposomes or particles can be prepared containing these lipidoids and therefore to achieve an effective delivery of the mRNA, as judged by the production of an encoded protein, following the injection of a lipidoid formulation via localized and/or systemic routes of administration. Lipidoid complexes of mRNAs can be administered by various means including, but not limited to, intravenous, intramuscular, or subcutaneous routes.

The synthesis of lipidoids is described in literature (see Mahon et al., Bioconjug. Chem. 2010 21:1448-1454; Schroeder et al., J Intern Med. 2010 267:9-21; Akinc et al., Nat Biotechnol. 2008 26:561-569; Love et al., Proc Natl Acad Sci USA. 2010 107:1864-1869; Siegwart et al., Proc Natl Acad Sci USA. 2011 108:12996-3001; all of which are incorporated herein in their entireties).

Formulations with the different lipidoids, including, but not limited to penta[3-(1-laurylaminopropionyl)]-triethylenetetramine hydrochloride (TETA-5LAP; also known as 98N12-5, see Murugaiah et al., Analytical Biochemistry, 401:61 (2010)), C₁₂-200 (including derivatives and variants), and MD1, can be tested for in vivo activity. The lipidoid “98N12-5” is disclosed by Akinc et al., Mol Ther. 2009 17:872-879. The lipidoid “C₁₂-200” is disclosed by Love et al., Proc Natl Acad Sci USA. 2010 107:1864-1869 and Liu and Huang, Molecular Therapy. 2010 669-670. Each of the references is herein incorporated by reference in its entirety.

In one embodiment, the mRNAs described herein can be formulated in an aminoalcohol lipidoid. Aminoalcohol lipidoids can be prepared by the methods described in U.S. Pat. No. 8,450,298 (herein incorporated by reference in its entirety).

The lipidoid formulations can include particles comprising either 3 or 4 or more components in addition to mRNAs. Lipidoids and mRNA formulations comprising lipidoids are described in Intl. Pub. No. WO 2015051214 (herein incorporated by reference in its entirety.

### Polypeptides of Interest

In some aspects, the present disclosure provides mRNAs (e.g., endonuclease-resistant mRNAs) comprising an open reading frame (ORF) encoding polypeptides of interest (e.g., therapeutic polypeptides). In some embodiments, the polypeptide of interest is a therapeutic polypeptide. In some embodiments, the disclosure provides method of generating an endonuclease-resistant mRNA comprising an ORF that encodes a polypeptide of interest (e.g., a therapeutic polypeptide), typically a protein or peptide having therapeutic properties for use in a subject. The polypeptides of interest can be essentially any protein or polypeptide that can be encoded by an mRNA.

In some embodiments, an mRNA of the disclosure encodes a polypeptide of interest that is a full-length protein. In some embodiments, an mRNA of the disclosure encodes a polypeptide of interest that is a functional fragment of a full-length protein (e.g., a fragment of the full-length protein that includes one or more functional domains such that the functional activity of the full-length protein is retained). In some embodiments, an mRNA of the disclosure encodes a polypeptide of interest that is not naturally occurring. In some embodiments, an mRNA of the disclosure encodes a polypeptide of interest that is a modified protein comprised of one or more heterologous domains (e.g., a protein that is a fusion protein comprised of one or more domains that do not naturally occur in the protein such that the function of the protein is altered).

Exemplary types of proteins (e.g., infectious disease antigens, tumor cell antigens, soluble effector molecules, antibodies, enzymes, recruitment factors, transcription factors, membrane bound receptors or ligands) that are encoded by an mRNA of the disclosure are described in detail in the following subsections.

### Naturally Occurring Targets

In some embodiments, an mRNA of the disclosure encodes a polypeptide of interest that is a naturally occurring target. In some embodiments, an mRNA encodes a polypeptide of interest that when expressed, modulates a naturally occurring target (e.g., up- or down-regulates the activity of a naturally occurring target). In some embodiments, a naturally occurring target is a soluble protein that is secreted by a cell. In some embodiments, a naturally occurring target is a protein that is retained within a cell (e.g., an intracellular protein). In some embodiments, a naturally occurring target is a membrane-bound or transmembrane protein. Non-limiting examples of naturally occurring targets include soluble proteins (e.g., chemokines, cytokines, growth factors, antibodies, enzymes), intracellular proteins (e.g., intracellular signaling proteins, transcription factors, enzymes, structural proteins) and membrane-bound or transmembrane proteins (e.g., receptors, adhesion molecules, enzymes).

In some embodiments, an mRNA encodes a polypeptide of interest that when expressed is a full-length naturally occurring target (i.e., a full-length protein). In some embodiments, an mRNA encodes a polypeptide of interest that when expressed is a fragment or portion of a naturally occurring target (i.e., a fragment or portion of a full-length protein). For example, in one embodiment, the protein or fragment thereof can be an immunogenic polypeptide that can be used as a vaccine.

In some embodiments, an mRNA encodes a polypeptide that when expressed, modulates a naturally occurring target (e.g., by encoding the target itself or by functioning to modulate the activity of the target). In some embodiments, a polypeptide of interest acts in an autocrine fashion, i.e., the polypeptide exerts an effect directly on the cell into which the mRNA is delivered. In some embodiments, an encoded polypeptide of interest acts in a paracrine fashion, i.e., the encoded polypeptide exerts an indirect effect on a cell that is not the cell into which the mRNA is delivered (e.g., delivery of the mRNA into one type of cell results in secretion of a molecule that exerts an effects on another type of cell, such as a bystander cell). In some embodiments, an encoded polypeptide of interest acts in both an autocrine fashion and a paracrine fashion.

### Naturally Occurring Soluble Targets

In some embodiments, an mRNA encodes a polypeptide of interest that modulates the activity of a naturally occurring soluble target, for example by encoding the soluble target itself or by modulating the expression (e.g., transcription or translation) of the soluble target. Non-limiting examples of naturally occurring soluble targets include cytokines, chemokines, growth factors, enzymes, and antibodies.

In some embodiments, an mRNA encoding a polypeptide of interest stimulates (e.g., upregulates, enhances) the activation or activity of a cell type, for example in situations where stimulation of an immune response is desirable, such as in cancer therapy or treatment of an infectious disease (e.g., a viral, bacterial, fungal, protozoal or parasitic infection). In another embodiment, an mRNA encoding a polypeptide of interest inhibits (e.g., downregulates, reduces) the activation or activity of a cell, for example in situations where inhibition of an immune response is desirable, such as in autoimmune diseases, allergies and transplantation.

In some embodiments, an mRNA of the disclosure encodes a soluble target that is a cytokine or chemokine with desirable uses for stimulating or inhibiting immune responses, e.g., that is useful in treating cancer as described further below.

In some embodiments, an mRNA of the disclosure encodes a soluble target that is a cytokine that stimulates the activation or activity of a cell such as an immune cell.

In some embodiments, an mRNA of the disclosure encodes a chemokine or a chemokine receptor which is useful for stimulating the activation or activity of an immune cell. Chemokines have been demonstrated to control the trafficking of inflammatory cells (including granulocytes and monocytes/monocytes), as well as regulating the movement of a wide variety of immune cells (including lymphocytes, natural killer cells and dendritic cells). Thus, chemokines are involved both in regulating inflammatory responses and immune responses. Moreover, chemokines have been shown to have effects on the proliferative and invasive properties of cancer cells (for a review of chemokines, see e.g., Mukaida, N. et al. (2014) Mediators of Inflammation, Article ID 170381, pg. 1-15).

In some embodiments, an mRNA of the disclosure encodes a recruitment factor which is useful to stimulate the homing, activation or activity of a cell. In one embodiment, the cell is an immune cell and the “recruitment factor” refers to a protein that promotes recruitment of an immune cell to a desired location (e.g., to a tumor site or an inflammatory site). For example, certain chemokines, chemokine receptors and cytokines have been shown to be involved in the recruitment of lymphocytes (see e.g., Oelkrug, C. and Ramage, J. M. (2014) Clin. Exp. Immunol. 178:1-8).

In some embodiments, an mRNA of the disclosure encodes an inhibitory cytokine or an antagonist of a stimulatory cytokine which is useful for inhibiting immune responses.

In some embodiments, an mRNA of the disclosure encodes a soluble target that is an antibody. As used herein, the term “antibody” refers to a whole antibody comprising two light chain polypeptides and two heavy chain polypeptides, or an antigen-binding fragment thereof. In some embodiments, a soluble target is a monoclonal antibody (e.g., full length monoclonal antibody) that displays a single binding specificity and affinity for a particular epitope. In some embodiments, a soluble target is an antigen binding fragment of a monoclonal antibody that retains the ability to bind a target antigen. Such fragments include, e.g., a single chain antibody, a single chain Fv fragment (scFv), an Fd fragment, an Fab fragment, an Fab′ fragment, or an F(ab′)₂ fragment.

In some embodiments, an mRNA of the disclosure encodes an antibody that recognizes a tumor antigen, against which a protective or a therapeutic immune response is desired, e.g., antigens expressed by a tumor cell. In some embodiments, a suitable antigen includes tumor associated antigens for the prevention or treatment of cancers.

In some embodiments, an mRNA of the disclosure encodes an antibody that recognizes an infectious disease antigen, against which protective or therapeutic immune responses are desired, e.g., an antigen present on a pathogen or infectious agent. In some embodiments, a suitable antigen includes an infectious disease associated antigen for the prevention or treatment of an infectious disease. Methods for identification of antigens on infectious disease agents that comprise protective epitopes (e.g., epitopes that when recognized by an antibody enable neutralization or blocking of infection caused by an infectious disease agent) are described in the art as detailed by Sharon, J. et al. (2013) Immunology 142:1-23. In some embodiments, an infectious disease antigen is present on a virus or on a bacterial cell.

In some embodiments, an mRNA of the disclosure encodes a soluble target that is a growth factor with desirable uses for modulating tissue healing and repair. A growth factor is a protein that stimulates the survival, growth, proliferation, migration or differentiation of cells, often for the purposes of promoting growth of lost tissue or enhancing the body's innate healing and repair mechanisms. In some embodiments, a growth factor is used to manipulate cells that include, but are not limited to, stromal cells (e.g., fibroblasts), immune cells, vascular cells (e.g., epithelial cells, platelets, pericytes), neural cells (e.g., astrocytes, neural stem cells, microglial cells), or bone cells (e.g., osteocyte, osteoblast, osteoclast, osteogenic cells).

In some embodiments, an mRNA of the disclosure encodes a soluble target that is an enzyme with desirable uses for modulating metabolism or growth in a subject. In some embodiments, an enzyme is administered to replace an endogenous enzyme that is absent or dysfunctional as described in Brady, R. et al, (2004) Lancet Neurol. 3:752. In some embodiments, an enzyme is used to treat a metabolic storage disease. A metabolic storage disease results from the systemic accumulation of metabolites due to the absence or dysfunction of an endogenous enzyme. Such metabolites include lipids, glycoproteins, and mucopolysaccharides. In some embodiments, an enzyme is used to reduce or eliminate the accumulation of monosaccharides, polysaccharides, glycoproteins, glycopeptides, glycolipids or lipids due to a metabolic storage disease.

### Naturally Occurring Intracellular Targets

In some embodiments, an mRNA of the disclosure encodes a polypeptide of interest that modulates the activity of a naturally occurring intracellular target, for example by encoding the intracellular target itself or by modulating the expression (e.g., transcription or translation) of the intracellular target in a cell. Non-limiting examples of naturally-occurring intracellular targets include transcription factors and cell signaling cascade molecules, including enzymes, that modulate cell growth, differentiation and communication. Additional examples include intracellular targets that regulate cell metabolism.

Suitable transcription factors and intracellular signaling cascade molecules for particular uses in stimulating or inhibiting cellular activity or responses are described in the art. In some embodiments, an mRNA of the disclosure encodes a transcription factor useful for stimulating the activation or activity of an immune cell. As used herein, a “transcription factor” refers to a DNA-binding protein that regulates the transcription of a gene. In some embodiments, an mRNA of the disclosure encodes a transcription factor that increases or polarizes an immune response.

In some embodiments, an mRNA of the disclosure encodes an intracellular adaptor protein (e.g., in a signal transduction pathway) useful for stimulating the activation or activity of a cell.

In some embodiments, an mRNA of the disclosure encodes an intracellular signaling protein useful for stimulating the activation or activity of a cell. In some embodiments, an mRNA of the disclosure encodes a tolerogenic transcription factor useful for inhibiting the activation or activity of an immune cell.

In some embodiments, an mRNA of the disclosure encodes an intracellular target that is a protein that is used to treat a metabolic disease or disorder.

In some embodiments, an mRNA of the disclosure encodes a polypeptide of interest that is a fully-functional mitochondrial protein (e.g., wild-type). In some embodiments, an mRNA of the disclosure encodes a mitochondrial protein encoded by mitochondrial DNA (e.g., a mitochondrial-encoded mitochondrial protein). In some embodiments, an mRNA of the disclosure encodes a mitochondrial protein encoded by nuclear DNA (e.g., a nuclear-encoded mitochondrial protein). In some embodiments, an mRNA of the disclosure is used to treat a mitochondrial disease resulting from a mutation in a mitochondrial protein. In some embodiments, translation of an mRNA encoding a mitochondrial protein provides sufficient quantity and/or activity of the protein to ameliorate a mitochondrial disease. In some embodiments, an mRNA encodes a polypeptide of interest that is a mitochondrial protein described in the MitoCarta2.0 mitochondrial protein inventory.

### Naturally Occurring Membrane Bound/Transmembrane Targets

In some embodiments, an mRNA of the disclosure encodes a polypeptide of interest that modulates the activity of a naturally-occurring membrane-bound/transmembrane target, for example by encoding the membrane-bound/transmembrane target itself or by modulating the expression (e.g., transcription or translation) of the membrane-bound/transmembrane target. Non-limiting examples of naturally-occurring membrane-bound/transmembrane targets include Cell surface receptors, growth factor receptors, costimulatory molecules, immune checkpoint molecules, homing receptors and HLA molecules.

In one embodiment, the membrane-bound/transmembrane targets are useful in stimulating or inhibiting immune responses are described herein. In some embodiments, an mRNA of the disclosure encodes a costimulatory factor that upregulates an immune response or is an antagonist of a costimulatory factor that downregulates an immune response. In some embodiments, an mRNA of the disclosure encodes an immune checkpoint protein that down-regulates immune cells (e.g., T cells).

In some embodiments, an mRNA of the disclosure encodes a membrane-bound/transmembrane protein target that serves as a homing signal.

In some embodiments, an mRNA of the disclosure encodes a membrane-bound/transmembrane protein target that is an immune receptor, e.g., on a lymphocyte or monocyte.

### Modified Targets

In some embodiments, an mRNA of the disclosure encodes a polypeptide of interest that is a modified polypeptide. In some embodiments, an mRNA of the disclosure encodes a polypeptide of interest that modulates a modified target (e.g., up- or down-regulates the activity of a non-naturally-occurring target). Typically, an mRNA of the disclosure encodes a modified target. Alternatively, if a cell expresses a modified target, an mRNA-encoded polypeptide functions to modulate the activity of the modified target in the cell. In some embodiments, a non-naturally occurring target is a full-length target, such as a full-length modified protein. In some embodiments, a non-naturally occurring target is a fragment or portion of a non-naturally-occurring target, such as a fragment or portion of a modified protein. In some embodiments, an mRNA-encoded polypeptide when expressed acts in an autocrine fashion to modulate a modified target, i.e., exerts an effect directly on the cell into which the mRNA is delivered. Additionally or alternatively, an mRNA-encoded polypeptide when expressed acts in a paracrine fashion to modulates a modified target, i.e., exerts an effect indirectly on a cell other than the cell into which the mRNA is delivered (e.g., delivery of the mRNA into one type of cell results in secretion of a molecule that exerts effects on another type of cell, such as bystander cells). Non-limiting examples of modified proteins include modified soluble proteins (e.g., secreted proteins), modified intracellular proteins (e.g., intracellular signaling proteins, transcription factors) and modified membrane-bound or transmembrane proteins (e.g., receptors).

### Modified Soluble Targets

In some embodiments, an mRNA of the disclosure encodes a polypeptide of interest that modulates a modified soluble target (e.g., up- or down-regulates the activity of a non-naturally-occurring soluble target). In some embodiments, an mRNA of the disclosure encodes a polypeptide of interest that is a modified soluble target. In some embodiments, a modified soluble target is a soluble protein that has been modified to alter (e.g., increase or decrease) the half-life (e.g., serum half-life) of the protein. Modified soluble proteins with altered half-life include modified cytokines and chemokines. In some embodiments, a modified soluble target is a soluble protein that has been modified to incorporate a tether such that the soluble protein becomes tethered to a cell surface. Modified soluble proteins incorporating a tether include tethered cytokines and chemokines.

### Modified Intracellular Targets

In some embodiments, an mRNA of the disclosure encodes a polypeptide of interest that modulates a modified intracellular target (e.g., up- or down-regulates the activity of a non-naturally-occurring intracellular target). In some embodiments, an mRNA of the disclosure encodes polypeptide of interest that is a modified intracellular target. In some embodiments, a modified intracellular target is a constitutively active mutant of an intracellular protein, such as a constitutively active transcription factor or intracellular signaling molecule. In some embodiments, a modified intracellular target is a dominant negative mutant of an intracellular protein, such as a dominant negative mutant of a transcription factor or intracellular signaling molecule. In some embodiments, a modified intracellular target is an altered (e.g., mutated) enzyme, such as a mutant enzyme with increased or decreased activity within an intracellular signaling cascade.

### Modified Membrane Bound/Transmembrane Targets

In some embodiments, an mRNA of the disclosure encodes a polypeptide of interest that modulates a modified membrane-bound/transmembrane target (e.g., up- or down-regulates the activity of a non-naturally-occurring membrane-bound/transmembrane target). In some embodiments, an mRNA of the disclosure encodes a polypeptide of interest that is a modified membrane-bound/transmembrane target. In some embodiments, a modified membrane-bound/transmembrane target is a constitutively active mutant of a membrane-bound/transmembrane protein, such as a constitutively active cell surface receptor (i.e., activates intracellular signaling through the receptor without the need for ligand binding). In some embodiments, a modified membrane-bound/transmembrane target is a dominant negative mutant of a membrane-bound/transmembrane protein, such as a dominant negative mutant of a cell surface receptor. In some embodiments, a modified membrane-bound/transmembrane target is a molecule that inverts signaling of a cellular synapse (e.g., agonizes or antagonizes signaling of a receptor). In some embodiments, a modified membrane-bound/transmembrane target is a chimeric membrane-bound/transmembrane protein, such as a chimeric cell surface receptor.

As used herein, the term “chimeric antigen receptor (CAR)” refers to an artificial transmembrane protein receptor comprising an extracellular domain capable of binding to a predetermined CAR ligand or antigen, an intracellular segment comprising one or more cytoplasmic domains derived from signal transducing proteins different from the polypeptide from which the extracellular domain is derived, and a transmembrane domain.

### Pharmaceutical Compositions

The present disclosure includes pharmaceutical compositions comprising an mRNA or a nanoparticle (e.g., a lipid nanoparticle) described herein, in combination with one or more pharmaceutically acceptable excipient, carrier or diluent. In particular embodiments, the mRNA is present in a nanoparticle, e.g., a lipid nanoparticle. In particular embodiments, the mRNA or nanoparticle is present in a pharmaceutical composition.

Pharmaceutical compositions may optionally include one or more additional active substances, for example, therapeutically and/or prophylactically active substances. Pharmaceutical compositions of the present disclosure may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference in its entirety). In particular embodiments, a pharmaceutical composition comprises an mRNA and a lipid nanoparticle, or complexes thereof.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may include between 0.1% and 100%, e.g., between 0.5% and 70%, between 1% and 30%, between 5% and 80%, or at least 80% (w/w) active ingredient.

The mRNAs of the disclosure can be formulated using one or more excipients to: (1) increase stability; (2) increase cell transfection; (3) permit the sustained or delayed release (e.g., from a depot formulation of the mRNA); (4) alter the biodistribution (e.g., target the mRNA to specific tissues or cell types); (5) increase the translation of a polypeptide encoded by the mRNA in vivo; and/or (6) alter the release profile of a polypeptide encoded by the mRNA in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, lipidoids, liposomes, lipid nanoparticles (e.g., liposomes and micelles), polymers, lipoplexes, core-shell nanoparticles, peptides, proteins, carbohydrates, cells transfected with mRNAs (e.g., for transplantation into a subject), hyaluronidase, nanoparticle mimics and combinations thereof. Accordingly, the formulations of the disclosure can include one or more excipients, each in an amount that together increases the stability of the mRNA, increases cell transfection by the mRNA, increases the expression of a polypeptide encoded by the mRNA, and/or alters the release profile of an mRNA-encoded polypeptide. Further, the mRNAs of the present disclosure may be formulated using self-assembled nucleic acid nanoparticles.

Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition. Excipients may include, for example: antiadherents, antioxidants, binders, coatings, compression aids, disintegrants, dyes (colors), emollients, emulsifiers, fillers (diluents), film formers or coatings, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

In some embodiments, the formulations described herein may include at least one pharmaceutically acceptable salt. Examples of pharmaceutically acceptable salts that may be included in a formulation of the disclosure include, but are not limited to, acid addition salts, alkali or alkaline earth metal salts, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

In some embodiments, the formulations described herein may contain at least one type of mRNA. As a non-limiting example, the formulations may contain 1, 2, 3, 4, 5 or more than 5 mRNAs described herein. In some embodiments, the formulations described herein may contain at least one mRNA encoding a polypeptide and at least one nucleic acid sequence such as, but not limited to, an siRNA, an shRNA, a snoRNA, and an miRNA.

Liquid dosage forms for e.g., parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and/or suspending agents. In certain embodiments for parenteral administration, compositions are mixed with solubilizing agents such as CREMAPHOR®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables. Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In some embodiments, pharmaceutical compositions including at least one mRNA described herein are administered to mammals (e.g., humans). Although the descriptions of pharmaceutical compositions provided herein are principally directed to pharmaceutical compositions which are suitable for administration to humans, it will be understood by the skilled artisan that such compositions are generally suitable for administration to any other animal, e.g., to a non-human mammal. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys. In particular embodiments, a subject is provided with two or more mRNAs described herein. In particular embodiments, the first and second mRNAs are provided to the subject at the same time or at different times, e.g., sequentially. In particular embodiments, the first and second mRNAs are provided to the subject in the same pharmaceutical composition or formulation, e.g., to facilitate uptake of both mRNAs by the same cells.

### Kits

In some embodiments, the disclosure provides a kit comprising an mRNA, or composition (e.g. lipid nanoparticle) comprising an mRNA as described herein. In some embodiments, a kit comprises a container comprising a pharmaceutical composition comprising a lipid nanoparticle comprising an mRNA described herein; and a pharmaceutically acceptable carrier, and a package insert comprising instructions for administration of the mRNA.

In some embodiments, a kit comprises a container comprising a pharmaceutical composition comprising a lipid nanoparticle comprising an mRNA encoding a polypeptide described herein; and a pharmaceutically acceptable carrier, and a package insert comprising instructions for administration of the mRNA and instruction for use in combination with a second composition comprising a second therapeutic agent.

In some embodiments, a kit comprises a container comprising a lipid nanoparticle encapsulating the mRNA described herein, and an optional pharmaceutically acceptable carrier, or a pharmaceutical composition, and a package insert comprising instructions for administration of the lipid nanoparticle or pharmaceutical composition. In some embodiments, a kit comprises a container comprising a lipid nanoparticle encapsulating the mRNA described herein, and an optional pharmaceutically acceptable carrier, or a pharmaceutical composition, and a package insert comprising instructions for administration of the lipid nanoparticle or pharmaceutical composition for treating or delaying progression of an disease or disorder in an individual. In some aspects, the package insert further comprises instructions for administration of the lipid nanoparticle or pharmaceutical composition in combination with a composition comprising a second therapeutic agent and an optional pharmaceutically acceptable carrier for treating or delaying progression of a disease or disorder in a patient.

In some embodiments, a kit comprises a medicament comprising a lipid nanoparticle encapsulating an mRNA described herein, and an optional pharmaceutically acceptable carrier, or a pharmaceutical composition, and a package insert comprising instructions for administration of the medicament alone or in combination with a composition comprising a second therapeutic agent and an optional pharmaceutically acceptable carrier.

### Devices

The present disclosure provides for devices that incorporate polynucleotides that encode polypeptides of interest. These devices contain in a stable formulation the reagents to synthesize a polynucleotide in a formulation available to be immediately delivered to a subject in need thereof, such as a human patient.

Devices for administration are employed to deliver the polynucleotides of the present disclosure according to single, multi- or split-dosing regimens taught herein. Such devices are taught in, for example, International Application PCT/US2013/30062 filed Mar. 9, 2013, the contents of which are incorporated herein by reference in their entirety.

Method and devices known in the art for multi-administration to cells, organs and tissues are contemplated for use in conjunction with the methods and compositions disclosed herein as embodiments of the present disclosure. These include, for example, those methods and devices having multiple needles, hybrid devices employing for example lumens or catheters as well as devices utilizing heat, electric current or radiation driven mechanisms.

According to the present disclosure, these multi-administration devices are utilized to deliver the single, multi- or split doses contemplated herein. Such devices are taught for example in, International Application PCT/US2013/30062 filed Mar. 9, 2013, the contents of which are incorporated herein by reference in their entirety.

In some embodiments, the polynucleotide is administered subcutaneously or intramuscularly via at least 3 needles to three different, optionally adjacent, sites simultaneously, or within a 60 minutes period (e.g., administration to 4, 5, 6, 7, 8, 9, or 10 sites simultaneously or within a 60 minute period).

Methods and Devices Utilizing Catheters and/or Lumens

Methods and devices using catheters and lumens are employed to administer the polynucleotides of the present disclosure on a single, multi- or split dosing schedule. Such methods and devices are described in International Application PCT/US2013/30062 filed Mar. 9, 2013 (Attorney Docket Number M300), the contents of which are incorporated herein by reference in their entirety.

### Methods and Devices Utilizing Electrical Current

Methods and devices utilizing electric current are employed to deliver the polynucleotides of the present disclosure according to the single, multi- or split dosing regimens taught herein. Such methods and devices are described in International Application PCT/US2013/30062 filed Mar. 9, 2013 (Attorney Docket Number M300), the contents of which are incorporated herein by reference in their entirety.

### Methods of Use

The disclosure provides methods using the mRNAs, compositions, lipid nanoparticles, or pharmaceutical compositions disclosed herein. In some aspects, the mRNAs described herein are used to increase the amount and/or quality of a polypeptide (e.g., a therapeutic polypeptide) encoded by and translated from the mRNA. In some embodiments, the mRNAs described herein are useful for increasing the expression of an encoded polypeptide of interest upon contacting cells with the mRNA or a composition comprising the mRNA. In some embodiments, the mRNAs described herein are useful for increasing the activity of an encoded polypeptide of interest that is transcribed upon contacting cells with the mRNA or a composition comprising the mRNA.

In some embodiments, the disclosure provides a method of increasing an amount of a polypeptide translated from an open reading frame comprising an mRNA (e.g., increased mRNA expression), the method comprising contacting a cell with an mRNA, a composition, a lipid nanoparticle, or a pharmaceutical composition according to the disclosure.

In some embodiments, the disclosure provides a method of increasing activity of a polypeptide translated from an mRNA, the method comprising contacting a cell with an mRNA, a composition, a lipid nanoparticle, or a pharmaceutical composition according to the disclosure.

In one embodiment, the method comprises administering to the subject a composition of the disclosure (or lipid nanoparticle thereof, or pharmaceutical composition thereof) comprising at least one mRNA construct encoding a polypeptide (e.g., a therapeutic polypeptide)

Compositions of the disclosure are administered to the subject at an effective amount or effective dose. In general, an effective amount of the composition will allow for efficient production of the encoded polypeptide in the cell. Metrics for efficiency may include polypeptide translation (indicated by polypeptide expression), polypeptide activity (indicated by levels of metabolic products, enzymatic products or biomarkers), and the level of mRNA degradation (indicated by mRNA stability or half-life).

The polynucleotides of the present disclosure (i.e., an mRNA comprising an ORF encoding a polypeptide of interest) are administered by any route that results in a therapeutically effective outcome. These include, but are not limited to enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration that is then covered by a dressing that occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal. In specific embodiments, compositions can be administered in a way that allows them cross the blood-brain barrier, vascular barrier, or other epithelial barrier. In some embodiments, a formulation for a route of administration can include at least one inactive ingredient. In some embodiments, the polynucleotides (e.g., mRNAs) of the present disclosure are delivered to a cell naked. As used herein in, “naked” refers to delivering polynucleotides free from agents that promote transfection. For example, in some aspects, the polynucleotides (e.g., mRNAs) delivered to the cell contain no modifications. The naked polynucleotides are delivered to the cell using routes of administration known in the art and described herein.

In some embodiments, the polynucleotides of the present disclosure are formulated, using the methods described herein. The formulations contain polynucleotides that are modified and/or unmodified. The formulations further include, but are not limited to, cell penetration agents, a pharmaceutically acceptable carrier, a delivery agent, a bioerodible or biocompatible polymer, a solvent, and a sustained-release delivery depot. The formulated polynucleotides are delivered to the cell using routes of administration known in the art and described herein.

In some embodiments, the compositions of the disclosure are formulated for direct delivery to an organ or tissue in any of several ways in the art including, but not limited to, direct soaking or bathing, via a catheter, by gels, powder, ointments, creams, gels, lotions, and/or drops, by using substrates such as fabric or biodegradable materials coated or impregnated with the compositions, and the like.

### Parenteral and Injectable Administration

The present disclosure encompasses the delivery of polynucleotides of the disclosure (e.g., an mRNA comprising an ORF encoding a polypeptide of interest) in forms suitable for parenteral and injectable administration. Liquid dosage forms for parenteral administration include, but are not limited to, pharmaceutically acceptable emulsions, microemulsions, solutions, suspensions, syrups, and/or elixirs. In addition to active ingredients, liquid dosage forms can comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof.

In some aspects, a pharmaceutical composition of the disclosure is formulated for parenteral administration comprising at least one inactive ingredient. Any or none of the inactive ingredients used have been approved by the US Food and Drug Administration (FDA). A non-exhaustive list of inactive ingredients for use in pharmaceutical compositions for parenteral administration includes hydrochloric acid, mannitol, nitrogen, sodium acetate, sodium chloride and sodium hydroxide.

Injectable preparations, for example, sterile injectable aqueous or oleaginous suspensions are formulated according to the known art using suitable dispersing agents, wetting agents, and/or suspending agents. Sterile injectable preparations are sterile injectable solutions, suspensions, and/or emulsions in nontoxic parenterally acceptable diluents and/or solvents, for example, as a solution in 1,3-butanediol. Among the acceptable vehicles and solvents that are employed are water, Ringer's solution, U.S.P., and isotonic sodium chloride solution. Sterile, fixed oils are conventionally employed as a solvent or suspending medium. For this purpose any bland fixed oil are employed including synthetic mono- or diglycerides. Fatty acids such as oleic acid are used in the preparation of injectables. In some embodiments, the sterile formulation also comprises adjuvants such as local anesthetics, preservatives and buffering agents.

In some aspects, injectable formulations are sterilized, for example, by filtration through a bacterial-retaining filter, and/or by incorporating sterilizing agents in the form of sterile solid compositions that are dissolved or dispersed in sterile water or other sterile injectable medium prior to use.

Injectable formulations are for direct injection into a region of a tissue, organ and/or subject. As a non-limiting example, a tissue, organ and/or subject can be directly injected a formulation by intramyocardial injection into the ischemic region. (See, e.g., Zangi et al. Nature Biotechnology 2013; the contents of which are herein incorporated by reference in its entirety).

In order to prolong the effect of an active ingredient, it is often desirable to slow the absorption of the active ingredient from subcutaneous or intramuscular injection. This is accomplished by the use of a liquid suspension of crystalline or amorphous material with poor water solubility. The rate of absorption of the drug then depends upon its rate of dissolution which, in turn, can depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release is controlled. Examples of other biodegradable polymers include poly(orthoesters) and poly(anhydrides). Depot injectable formulations are prepared by entrapping the drug in liposomes or microemulsions that are compatible with body tissues.

### Therapeutic Methods

The methods of the disclosure for preparing a therapeutic mRNA further comprise treating or delaying progression of a disease or disorder in a clinical, prophylactic or therapeutic application that would benefit from expression of a therapeutic polypeptide encoded by the mRNA. For example, a disease or disorder that would benefit from increased expression of a membrane bound protein, an intracellular protein, or a secreted protein (such as cancer, an autoimmune disease, an infectious disease, a metabolic disease). In one embodiment, the protein is an immunogenic protein or polypeptide, e.g., an infectious disease antigen, a tumor cell antigen. In another embodiment, the protein is a soluble effector molecule, an antibody, an enzyme, a recruitment factor, a transcription factor, a membrane bound receptor, a membrane bound ligand or any fragment or variant thereof. A method of treating a patient with a disease or disorder that would benefit from increased expression of a therapeutic polypeptide comprises providing to the subject an effective amount of an mRNA that comprises an ORF encoding the therapeutic polypeptide.

In some embodiments, the polynucleotides of the present disclosure are used in the preparation, manufacture and therapeutic use of polynucleotide molecules comprising a polynucleotide sequence encoding a polypeptide of interest (e.g., a membrane bound, intracellular, or secreted protein). In some embodiments, the polynucleotides of the present disclosure can be used to treat and/or prevent diseases, disorders or conditions that would benefit from increased expression of a therapeutic polypeptide. Typically, but not exclusively, the polynucleotides of the present disclosure can be used to treat and/or prevent a disease or disorder resulting from a deficiency of a polypeptide of interest (e.g., a membrane bound, intracellular, or secreted protein). In some embodiments, the nucleotides are used in methods for reducing the levels of a biomarker of a therapeutic polypeptide (e.g., a membrane bound, intracellular, or secreted protein) in a subject in need thereof. For instance, one aspect of the disclosure provides a method of alleviating the symptoms of a disease or disorder resulting from an enzyme deficiency in a subject via the administration of a composition comprising a polynucleotide encoding the cellular enzyme to that subject.

In some embodiments, the polynucleotides of the present disclosure are used to reduce the level of a metabolite associated with a disease or disorder that would benefit from increased expression of a therapeutic polypeptide, the method comprising administering to the subject an effective amount of a polynucleotide encoding the therapeutic polypeptide (e.g., a membrane bound, intracellular, or secreted protein). In some embodiments, the administration of an effective amount of a polynucleotide reduces the levels of a biomarker or a combination of biomarkers of the disease or disorder in a subject. In some embodiments, the administration of the polynucleotide results in reduction in the level of one or more biomarkers of the disease or disorder within a short period of time after administration of the polynucleotide.

In some embodiments, the administration of the polynucleotide results in expression of a therapeutic polypeptide (e.g., a membrane bound, intracellular, or secreted protein) described herein, in cells of the subject. In some embodiments, administering the polynucleotide encoding a therapeutic polypeptide that is a membrane bound, intracellular, or secreted protein results in an increase of protein enzymatic activity in the subject. For example, in some embodiments, the polynucleotides of the present disclosure are used in methods of administering a composition comprising a polynucleotide sequence encoding a therapeutic polypeptide that is a membrane bound, intracellular, or secreted protein to a subject, wherein the method results in an increase of protein enzymatic activity in at least some cells of a subject. In some embodiments, the administration of a composition comprising a polynucleotide sequence encoding a therapeutic polypeptide that is a membrane bound, intracellular, or secreted protein to a subject results in an increase of protein enzymatic activity in cells of subject by at least 10%, at least 25%, at least 50%, at least 75%, at least 100%, or by more than 100%. In some embodiments, the administration of a composition comprising a polynucleotide sequence encoding a therapeutic polypeptide that is a membrane bound, intracellular, or secreted protein to a subject results in an increase of protein enzymatic activity in cells subject to a level at least 10%, at least 20%, at least 30%, at least 40%, at least 50%, at least 60%, at least 70%, at least 80%, at least 90%, or to 100% or more of the activity level expected in a normal subject, e.g., a normal human not suffering from the disease or disorder resulting from an enzyme deficiency. In some embodiments, the administration of the polynucleotide results in expression of a polypeptide of interest that is a membrane bound, intracellular, or secreted protein in at least some of the cells of a subject that persists for a period of time sufficient to allow significant enzymatic activity to occur.

In some embodiments, the expression of the encoded therapeutic polypeptide is increased. In some embodiments, the IVT polynucleotide increases protein expression levels in cells when introduced into those cells, e.g., by 20-50%, at least 20%, at least 25%, at least 30%, at least 35%, at least 40%, at least 45%, or at least 50%.

Other aspects of the present disclosure relate to transplantation of cells containing polynucleotides to a mammalian subject. Administration of cells to mammalian subjects is known to those of ordinary skill in the art, and includes, but is not limited to, local implantation (e.g., topical or subcutaneous administration), organ delivery or systemic injection (e.g., intravenous injection or inhalation), and the formulation of cells in pharmaceutically acceptable carriers. Such compositions containing polynucleotides are formulated for administration intramuscularly, transarterially, intraperitoneally, intravenously, intranasally, subcutaneously, endoscopically, transdermally, or intrathecally.

In some embodiments, the composition is formulated for extended release.

In some embodiments, the concentration of a biomarker of a therapeutic polypeptide (e.g., a membrane bound, intracellular, or secreted protein) in biological specimens (e.g., blood, plasma, serum, cerebral spinal fluid, urine, or any other biofluids) of a patient is monitored before, during and/or after a course of treatment involving the administration of a polynucleotide (e.g., mRNA) encoding a therapeutic polypeptide (e.g., a membrane bound, intracellular, or secreted protein) or a pharmaceutical composition thereof to the patient. In some embodiments, the concentration of a biomarker of a therapeutic polypeptide (e.g., a membrane bound, intracellular, or secreted protein) in biological specimens (e.g., urine, blood, plasma, serum, cerebral spinal fluid, or any other biofluids) of a patient is monitored before, during and/or after a course of treatment involving the administration of a polynucleotide (e.g., mRNA) encoding a therapeutic polypeptide (e.g., a membrane bound, intracellular, or secreted protein) or a pharmaceutical composition thereof to the patient. In some embodiments, the dosage, frequency and/or length of administration of a polynucleotide (e.g., mRNA) encoding a therapeutic polypeptide (e.g., a membrane bound, intracellular, or secreted protein) or a pharmaceutical composition thereof to a patient is modified as a result of the concentration of a biomarker of the therapeutic polypeptide (e.g., a membrane bound, intracellular, or secreted protein). Alternatively, changes in one or more of these monitoring parameters (e.g., concentration of a biomarker) might indicate that the course of treatment involving the administration of the polynucleotide (e.g., mRNA) encoding a therapeutic polypeptide (e.g., a membrane bound, intracellular, or secreted protein) or pharmaceutical composition thereof is effective in treating a disease or disorder that would benefit from increased expression of a therapeutic polypeptide (e.g., a membrane bound, intracellular, or secreted protein).

In a specific embodiment, presented herein is a method for treating a disease or disorder that would benefit from increased expression of a therapeutic polypeptide (e.g., a membrane bound, intracellular, or secreted protein), comprising: (a) administering to a patient in need thereof one or more doses of a polynucleotide (e.g., mRNA) encoding a therapeutic polypeptide (e.g., a membrane bound, intracellular, or secreted protein) or a pharmaceutical composition thereof; and (b) monitoring the concentration of a biomarker of the therapeutic polypeptide (e.g., detected in biological specimens such as plasma, serum, cerebral spinal fluid, urine, or other biofluids). In some embodiments, an increase in the biomarker following administration of the polynucleotide (e.g., mRNA) encoding a therapeutic polypeptide (e.g., a membrane bound, intracellular, or secreted protein) or pharmaceutical composition thereof indicates that the course of treatment is effective for treating the disease or disorder that would benefit from increased expression of a therapeutic polypeptide (e.g., a membrane bound, intracellular, or secreted protein).

The concentration of a biomarker of a therapeutic polypeptide (e.g., a membrane bound, intracellular, or secreted protein) of a patient is detected by any technique known to one of skill in the art. In some embodiments, the method for detecting the concentration of the biomarker in a patient involves obtaining a tissue or fluid sample from the patient and detecting the concentration of the biomarker in the biological sample (e.g., from plasma serum sample, cerebral spinal fluid, urine, or other biofluids) that has been subjected to certain types of treatment (e.g., centrifugation) and detection by use of, e.g., standard gas chromatography/mass spectroscopy (GC/MS) stable-isotope dilution methods, positive chemical ionization gas chromatography mass spectrometry (CI GC-MS) spectroscopic techniques (e.g., UV spectroscopy) or high pressure liquid chromatography (HPLC).

In some embodiments, the methods for treating a disease or disorder that would benefit from increased expression of a therapeutic polypeptide (e.g., a membrane bound, intracellular, or secreted protein) provided herein alleviate or manage one, two or more symptoms associated with the disease or disorder. In some embodiments, alleviating or managing one, two or more symptoms of a treating a disease or disorder that would benefit from increased expression of a therapeutic polypeptide (e.g., a membrane bound, intracellular, or secreted protein) is used as a clinical endpoint for efficacy of a polynucleotide for treating the disease or disorder. In some embodiments, the methods for treating a treating a disease or disorder that would benefit from increased expression of a therapeutic polypeptide (e.g., a membrane bound, intracellular, or secreted protein) provided herein reduce the duration and/or severity of one or more symptoms associated with the disease or disorder. In some embodiments, the methods for treating a disease or disorder that would benefit from increased expression of a therapeutic polypeptide (e.g., a membrane bound, intracellular, or secreted protein) provided herein inhibit the onset, progression and/or recurrence of one or more symptoms associated with the disease or disorder. In some embodiments, the methods for treating a treating a disease or disorder that would benefit from increased expression of a therapeutic polypeptide (e.g., a membrane bound, intracellular, or secreted protein) provided herein reduce the number of symptoms associated with the disease or disorder. In some embodiments, the methods for treating a treating a disease or disorder that would benefit from increased expression of a therapeutic polypeptide (e.g., a membrane bound, intracellular, or secreted protein) provided herein inhibit or reduce the progression of one or more symptoms associated therewith.

In some embodiments, the methods for treating a treating a disease or disorder that would benefit from increased expression of a therapeutic polypeptide (e.g., a membrane bound, intracellular, or secreted protein) provided herein reduce hospitalization (e.g., the frequency or duration of hospitalization) of a patient diagnosed with the disease or disorder. In some embodiments, the methods for treating a treating a disease or disorder that would benefit from increased expression of a therapeutic polypeptide (e.g., a membrane bound, intracellular, or secreted protein) provided herein reduce hospitalization length of a patient diagnosed with the disease or disorder. In some embodiments, the methods for treating a treating a disease or disorder that would benefit from increased expression of a therapeutic polypeptide (e e.g., a membrane bound, intracellular, or secreted protein) provided herein decrease the hospitalization rate.

In some embodiments, the methods provided herein increase the survival of a patient diagnosed with a disease or disorder that would benefit from increased expression of a therapeutic polypeptide (e.g., a membrane bound, intracellular, or secreted protein). In some embodiments, the methods for treating a disease or disorder that would benefit from increased expression of a therapeutic polypeptide (e.g., a membrane bound, intracellular, or secreted protein) provided herein reduce the mortality of subjects diagnosed with the disease or disorder. In some embodiments, the methods for treating a disease or disorder that would benefit from increased expression of a therapeutic polypeptide (e.g., a membrane bound, intracellular, or secreted protein) provided herein increase symptom-free survival of patients having the disease or disorder. In some embodiments, the methods for treating a disease or disorder that would benefit from increased expression of a therapeutic polypeptide (e.g., a membrane bound, intracellular, or secreted protein) provided herein do not cure the disease or disorder in patients, but prevent the progression or worsening of the disease. In some embodiments, the methods for treating a disease or disorder that would benefit from increased expression of a therapeutic polypeptide (e.g., a membrane bound, intracellular, or secreted protein) provided herein enhance or improve the therapeutic effect of another therapy.

In some embodiments, the methods for treating a disease or disorder that would benefit from increased expression of a therapeutic polypeptide (e.g., a membrane bound, intracellular, or secreted protein) provided herein reduce the concentration of a biomarker of the therapeutic polypeptide (e.g., a membrane bound, intracellular, or secreted protein) in a subject by at least about 5%, 10%, 15%, 20%, 25%, 30%, 35%, 40%, 45%, 50%, 55%, 60%, 65%, 80%, 85%, 90%, 95%, or 100%, or in a range of from 5% to 50%, 10% to 50%, 20% to 50%, 20% to 75%, 25% to 75%, 25% to 90% or 10% to 99% relative to the respective concentration prior to administration of a polynucleotide (e.g., mRNA) encoding the therapeutic polypeptide (e.g., a membrane bound, intracellular, or secreted protein), as assessed by methods well known in the art or described herein.

In some embodiments, the methods for treating a disease or disorder that would benefit from increased expression of a therapeutic polypeptide (e.g., a membrane bound, intracellular, or secreted protein) provided herein reduce the concentration of a metabolite of a therapeutic polypeptide (e.g., a membrane bound, intracellular, or secreted protein) in a subject by at least about 5%, 10%, 15%, 20%, 25%, 30%, 35%, 40%, 45%, 50%, 55%, 60%, 65%, 80%, 85%, 90%, 95%, or 100%, or in a range of from 5% to 50%, 10% to 50%, 20% to 50%, 20% to 75%, 25% to 75%, 25% to 90% or 10% to 99% relative to the respective concentration prior to administration of a polynucleotide (e.g., mRNA) encoding the therapeutic polypeptide (e.g., a membrane bound, intracellular, or secreted protein), as assessed by methods well known in the art or described herein.

In some embodiments, the methods for treating a disease or disorder that would benefit from increased expression of a therapeutic polypeptide (e.g., a membrane bound, intracellular, or secreted protein) provided herein increase the cellular enzyme activity in a subject by at least about 5%, 10%, 15%, 20%, 25%, 30%, 35%, 40%, 45%, 50%, 55%, 60%, 65%, 80%, 85%, 90%, 95%, or 100%, or in a range of from 10% to 50%, 20% to 50%, 20% to 75%, 25% to 75%, 25% to 90% or 10% to 99% relative to the respective concentration prior to administration of a polynucleotide (e.g., mRNA) encoding the therapeutic polypeptide, as assessed by methods well known in the art or described herein. In certain embodiment, the increase in cellular enzyme activity is determined by obtaining cells (e.g., fibroblasts or lymphocytes) from the subject, culturing the cells in the presence or absence of a polynucleotide, and comparing the cellular enzyme activity in the presence of the polynucleotide (e.g., mRNA) encoding the therapeutic polypeptide to the cellular enzyme activity in the absence of the polynucleotide. Techniques for measuring cellular enzyme activity are known in the art and described herein.

In some aspects, the methods for treating a disease or disorder that would benefit from increased expression of a therapeutic polypeptide (e.g., a membrane bound, intracellular, or secreted protein) provided herein improve developmental or cognitive function in a subject. In some aspects, the methods for treating a disease or disorder that would benefit from increased expression of a therapeutic polypeptide (e.g., a membrane bound, intracellular, or secreted protein) provided herein improve control of muscle contractions by a subject as assessed by methods well known in the art. In some embodiments, the methods for treating a disease or disorder that would benefit from increased expression of a therapeutic polypeptide (e.g., a membrane bound, intracellular, or secreted protein) provided herein improve renal function. In some embodiments, the methods for treating a disease or disorder that would benefit from increased expression of a therapeutic polypeptide (e.g., a membrane bound, intracellular, or secreted protein) provided herein decrease the need for kidney transplant, liver transplant or both. In some embodiments, the methods for treating a disease or disorder that would benefit from increased expression of a therapeutic polypeptide (e.g., a membrane bound, intracellular, or secreted protein) provided herein decrease the requirement for hospitalization. In some embodiments, the methods for treating a disease or disorder that would benefit from increased expression of a therapeutic polypeptide (e.g., a membrane bound, intracellular, or secreted protein) provided herein decrease the length and/or frequency of hospitalization.

In some embodiments, the subject is a male human. In some embodiments, the subject is a female human. In some embodiments, a subject treated for a disease or disorder that would benefit from increased expression of a therapeutic polypeptide (e.g., a membrane bound, intracellular, or secreted protein) in accordance with the methods provided herein is a fetus. In accordance with this embodiment, a pregnant female is administered a polynucleotide in a manner that permits the polynucleotide to pass through the placenta to the fetus. Alternatively, the polynucleotide is administered directly to the fetus by, e.g., injection. In some embodiments, a subject treated for a disease or disorder that would benefit from increased expression of a therapeutic polypeptide (e.g., a membrane bound, intracellular, or secreted protein) in accordance with the methods provided herein is a human infant. In one embodiment, a subject treated for a disease or disorder that would benefit from increased expression of a therapeutic polypeptide (e.g., a membrane bound, intracellular, or secreted protein) in accordance with the methods provided herein is an elderly human. In another embodiment, a subject treated for a disease or disorder that would benefit from increased expression of a therapeutic polypeptide (e.g., a membrane bound, intracellular, or secreted protein) in accordance with the methods provided herein is a human adult. In another embodiment, a subject treated for a disease or disorder that would benefit from increased expression of a therapeutic polypeptide (e.g., a membrane bound, intracellular, or secreted protein) in accordance with the methods provided herein is a human child. In another embodiment, a subject treated for a a disease or disorder that would benefit from increased expression of a therapeutic polypeptide (e.g., a membrane bound, intracellular, or secreted protein) in accordance with the methods provided herein is a human toddler.

In a specific embodiment, a subject treated for a disease or disorder that would benefit from increased expression of a therapeutic polypeptide (e.g., a membrane bound, intracellular, or secreted protein) in accordance with the methods provided herein is a human that is less than 5 years old. In another specific embodiment, a subject treated for a disease or disorder that would benefit from increased expression of a therapeutic polypeptide (e.g., a membrane bound, intracellular, or secreted protein) in accordance with the methods provided herein is a human that is older than 5 years old. In a specific embodiment, a subject treated for a disease or disorder that would benefit from increased expression of a therapeutic polypeptide (e.g., a membrane bound, intracellular, or secreted protein) in accordance with the methods provided herein is a human that is less than 5 years old, is older than 5 years old, is 18 years old or is older than 18 years old.

In some embodiments, a subject treated for a disease or disorder that would benefit from increased expression of a therapeutic polypeptide (e.g., a membrane bound, intracellular, or secreted protein) in accordance with the methods provided herein is administered a polynucleotide (e.g., mRNA) encoding a therapeutic polypeptide or a pharmaceutical composition thereof, or a combination therapy before any adverse effects or intolerance to therapies other than the polynucleotide develops. In some embodiments, a subject treated for a disease or disorder that would benefit from increased expression of a therapeutic polypeptide (e.g., a membrane bound, intracellular, or secreted protein) in accordance with the methods provided herein is a refractory patient. In a certain embodiment, a refractory patient is a patient that is refractory to a standard therapy.

In some embodiments, a subject treated for a disease or disorder that would benefit from increased expression of a therapeutic polypeptide (e.g., a membrane bound, intracellular, or secreted protein) in accordance with the methods provided herein is a human that has proven refractory to therapies other than treatment with a polynucleotide, but is no longer on these therapies. In some embodiments, a subject treated for a disease or disorder that would benefit from increased expression of a therapeutic polypeptide (e.g., a membrane bound, intracellular, or secreted protein) in accordance with the methods provided herein is a human already receiving one or more conventional therapies.

In some embodiments, a subject treated for a disease or disorder that would benefit from increased expression of a therapeutic polypeptide (e.g., a membrane bound, intracellular, or secreted protein) in accordance with the methods provided herein is a human susceptible to adverse reactions to conventional therapies. In some embodiments, a subject treated for a disease or disorder that would benefit from increased expression of a therapeutic polypeptide (e.g., a membrane bound, intracellular, or secreted protein) in accordance with the methods provided herein is a human that has not received a therapy, e.g., a prior to the administration of a polynucleotide or a pharmaceutical composition thereof. In some embodiments, a subject treated for a disease or disorder that would benefit from increased expression of a therapeutic polypeptide (e.g., a membrane bound, intracellular, or secreted protein) in accordance with the methods provided herein is a human that has received a therapy prior to administration of a polynucleotide or a pharmaceutical composition thereof. In some embodiments, a subject treated for a disease or disorder that would benefit from increased expression of a therapeutic polypeptide (e.g., a membrane bound, intracellular, or secreted protein) in accordance with the methods provided herein is a human that has experienced adverse side effects to the prior therapy or the prior therapy was discontinued due to unacceptable levels of toxicity to the human.

### Dosage and Administration

Accordingly, in one aspect, the disclosure pertains to a method of increased expression of a therapeutic polypeptide in a subject in need thereof, the method comprising administering to the subject a composition of the disclosure comprising an mRNA comprising an ORF encoding the therapeutic polypeptide. In some embodiments, the subject is provided with or administered a nanoparticle (e.g., a lipid nanoparticle) comprising the mRNA. In some embodiments, the subject is provided with or administered a pharmaceutical composition of the disclosure comprising the mRNA. In some embodiments, the pharmaceutical composition comprises an mRNA encoding a therapeutic polypeptide described herein, and optionally it comprises a nanoparticle comprising the mRNA. In some embodiments, the mRNA, nanoparticle, or pharmaceutical composition is administered to the patient parenterally. In particular embodiments, the subject is a mammal, e.g., a human. In various embodiments, the subject is provided with an effective amount of the mRNA.

A pharmaceutical composition including one or more therapeutic mRNAs of the disclosure may be administered to a subject by any suitable route. In some embodiments, compositions of the disclosure are administered by one or more of a variety of routes, including parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g. by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. In some embodiments, a composition may be administered intravenously, intramuscularly, intradermally, intra-arterially, intratumorally, subcutaneously, or by inhalation. However, the present disclosure encompasses the delivery of compositions of the disclosure by any appropriate route taking into consideration likely advances in the sciences of drug delivery. In general, the most appropriate route of administration will depend upon a variety of factors including the nature of the pharmaceutical composition including one or more mRNAs (e.g., its stability in various bodily environments such as the bloodstream and gastrointestinal tract), and the condition of the patient (e.g., whether the patient is able to tolerate particular routes of administration).

In certain embodiments, compositions of the disclosure may be administered that delivers a therapeutic mRNA of the disclosure at a dosage level from about 0.0001 mg/kg to about 10 mg/kg, from about 0.001 mg/kg to about 10 mg/kg, from about 0.005 mg/kg to about 10 mg/kg, from about 0.01 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.1 mg/kg to about 10 mg/kg, from about 1 mg/kg to about 5 mg/kg, from about 2 mg/kg to about 5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, or from about 0.1 mg/kg to about 1 mg/kg in a given dose, where a dose of 1 mg/kg provides 1 mg of mRNA or nanoparticle per 1 kg of subject body weight. In some embodiments, a dose of about 0.005 mg/kg to about 5 mg/kg of mRNA or nanoparticle of the disclosure may be administrated.

In some embodiments, the therapeutic composition comprises a dosage of a therapeutic mRNA of the disclosure that is 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 100-300 μg, 120-250 μg, 150-250 μg, 180-280 μg, 200-300 μg, 30-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, or 300-400 μg per dose. In some embodiments, the therapeutic composition is administered to the subject by intradermal or intramuscular injection. In some embodiments, the therapeutic composition is administered to the subject on day zero. In some embodiments, a second dose of the therapeutic composition is administered to the subject on day seven, or day fourteen or day twenty one.

In some embodiments, a dosage of 25 micrograms of a therapeutic mRNA of the disclosure is included in the therapeutic composition administered to the subject. In some embodiments, a dosage of 10 micrograms of a therapeutic mRNA of the disclosure is included in the therapeutic composition administered to the subject. In some embodiments, a dosage of 30 micrograms of a therapeutic mRNA of the disclosure is included in the therapeutic composition administered to the subject. In some embodiments, a dosage of 100 micrograms of a therapeutic mRNA of the disclosure is included in the therapeutic composition administered to the subject. In some embodiments, a dosage of 50 micrograms of a therapeutic mRNA of the disclosure is included in the therapeutic composition administered to the subject. In some embodiments, a dosage of 75 micrograms of a therapeutic mRNA of the disclosure is included in the therapeutic composition administered to the subject. In some embodiments, a dosage of 150 micrograms of a therapeutic mRNA of the disclosure is included in the therapeutic composition administered to the subject. In some embodiments, a dosage of 400 micrograms of a therapeutic mRNA of the disclosure is included in the therapeutic composition administered to the subject. In some embodiments, a dosage of 300 micrograms of a therapeutic mRNA of the disclosure is included in the therapeutic composition administered to the subject. In some embodiments, a dosage of 200 micrograms of a therapeutic mRNA of the disclosure is included in the therapeutic composition administered to the subject. In some embodiments, the therapeutic composition is chemically modified and in other embodiments the therapeutic composition is not chemically modified.

In some embodiments, the effective amount is a total dose of 1-100 μg of a therapeutic mRNA of the disclosure. In some embodiments, the effective amount is a total dose of 100 μg of a therapeutic mRNA of the disclosure. In some embodiments, the effective amount is a dose of 25 μg of a therapeutic mRNA of the disclosure administered to the subject a total of one or two times. In some embodiments, the effective amount is a dose of 100 μg of a therapeutic mRNA of the disclosure administered to the subject a total of two times. In some embodiments, the effective amount is a dose of 1 μg-10 μg, 1 μg-20 μg, 1 μg-30 μg, 5 μg-10 μg, 5 μg-20 μg, 5 μg-30 μg, 5 μg-40 μg, 5 μg-50 μg, 10 μg-15 μg, 10 μg-20 μg, 10 μg-25 μg, 10 μg-30 μg, 10 μg-40 μg, 10 μg-50 μg, 10 μg-60 μg, 15 μg-20 μg, 15 μg-25 μg, 15 μg-30 μg, 15 μg-40 μg, 15 μg-50 μg, 20 μg-25 μg, 20 μg-30 μg, 20 μg-40 μg 20 μg-50 μg, 20 μg-60 μg, 20 μg-70 μg, 20 μg-75 μg, 30 μg-35 μg, 30 μg-40 μg, 30 μg-45 μg 30 μg-50 μg, 30 μg-60 μg, 30 μg-70 μg, 30 μg-75 μg of a therapeutic mRNA of the disclosure which may be administered to the subject a total of one or two times or more.

A dose may be administered one or more times per day, in the same or a different amount, to obtain a desired level of mRNA expression and/or effect (e.g., a therapeutic effect). The desired dosage may be delivered, for example, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). For example, in certain embodiments, a composition of the disclosure is administered at least two times wherein the second dose is administered at least one day, or at least 3 days, or least 7 days, or at least 10 days, or at least 14 days, or at least 21 days, or at least 28 days, or at least 35 days, or at least 42 days or at least 48 days after the first dose is administered. In certain embodiments, a first and second dose are administered on days 0 and 2, respectively, or on days 0 and 7 respectively, or on days 0 and 14, respectively, or on days 0 and 21, respectively, or on days 0 and 48, respectively. Additional doses (i.e., third doses, fourth doses, etc.) can be administered on the same or a different schedule on which the first two doses were administered. For example, in some embodiments, the first and second dosages are administered 7 days apart and then one or more additional doses are administered weekly thereafter. In another embodiment, the first and second dosages are administered 7 days apart and then one or more additional doses are administered every two weeks thereafter.

In some embodiments, a single dose may be administered, for example, prior to or after a surgical procedure or in the instance of an acute disease, disorder, or condition. The specific therapeutically effective, prophylactically effective, or otherwise appropriate dose level for any particular patient will depend upon a variety of factors including the severity and identify of a disorder being treated, if any; the one or more mRNAs employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

In some embodiments, the present disclosure provides methods of administration of LNP encapsulated mRNAs encoding a therapeutic polypeptide for the treatment of a disease or disorder, such as those described herein. In some embodiments, the methods described herein comprise administering to the subject an LNP encapsulating an mRNA of the disclosure encoding a therapeutic polypeptide (e.g., a therapeutic polypeptide).

Compositions of the disclosure are administered to the subject in an effective amount. In general, an effective amount of the composition will allow for efficient production of the encoded polypeptide in cells of the subject. Metrics for efficiency may include polypeptide translation (indicated by polypeptide expression), polypeptide activity (indicated by levels of metabolic products, enzymatic products or biomarkers), and the level of mRNA degradation (indicated by mRNA stability or half-life).

In some embodiments, a subject is administered at least one mRNA composition described herein. In related embodiments, the subject is provided with or administered a nanoparticle (e.g., a lipid nanoparticle) comprising the mRNA(s). In further related embodiments, the subject is provided with or administered a pharmaceutical composition of the disclosure to the subject. In particular embodiments, the pharmaceutical composition comprises an mRNA(s) as described herein, or it comprises a nanoparticle comprising the mRNA(s). In particular embodiments, the mRNA(s) is present in a nanoparticle, e.g., a lipid nanoparticle. In particular embodiments, the mRNA(s) or nanoparticle is present in a pharmaceutical composition.

Suitable doses for human patients can be evaluated in, e.g., a Phase I dose escalation study. Data obtained from the cell culture assays and animal studies can be used in formulating a range of dosage for use in humans. The dosage of such mRNA described herein lies generally within a range of local concentrations of the mRNA that include the ED50 with little or no toxicity. The dosage may vary within this range depending upon the dosage form employed and the route of administration utilized. For the mRNA and compositions described herein, the therapeutically effective dose can be estimated initially from cell culture assays. A dose can be formulated in animal models to achieve a therapeutically effective concentration within the local site that includes the IC50 (i.e., the concentration of the mRNA which achieves a half-maximal inhibition of symptoms) as determined in cell culture. Such information can be used to more accurately determine useful doses in humans.

In some embodiments, the mRNA or composition is administered as a single dose or as two or more doses (which may or may not contain the same amount of the desired molecule) over time, or as a continuous infusion via an implantation device or catheter. Further refinement of the appropriate dosage is routinely made by those of ordinary skill in the art. In certain embodiments, appropriate dosages can be ascertained through use of appropriate dose-response data. In some embodiments, the specified time period is determined by a clinician.

In some embodiments, the dosing regimen is determined by the pharmacodynamics effects of the therapeutic polypeptide. In some embodiments, the frequency of dosing will take into account the pharmacokinetic parameters of the mRNA in the formulation used. In certain embodiments, a clinician will administer the composition until a dosage is reached that achieves or maintains the desired effect. In some embodiments, achievement of a desired effect occurs immediately after administration of a dose. In some embodiments, achievement occurs at any point in time following administration. In some embodiments, achievement occurs at any point in time during a dosing interval. In some embodiments, achievement of a desired effect is determined by analyzing a biological sample (e.g., biopsy) immediately after administration of a dose, at any point in time following administration of a dose, at any point in time during a doing interval, or combinations thereof.

### Combination Therapy

In some embodiments, a pharmaceutical composition of the disclosure may be administered in combination with another agent, for example, another therapeutic agent, a prophylactic agent, and/or a diagnostic agent. By “in combination with,” it is not intended to imply that the agents must be administered at the same time and/or formulated for delivery together, although these methods of delivery are within the scope of the present disclosure. For example, one or more compositions including one or more different mRNAs may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions of the disclosure, or imaging, diagnostic, or prophylactic compositions thereof in combination with agents that improve their bioavailability, reduce and/or modify their metabolism, inhibit their excretion, and/or modify their distribution within the body.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects).

Presented herein are combination therapies for the treatment of a disease or disorder that would benefit from increased expression of a therapeutic polypeptide which involve the administration of a polynucleotide (e.g., mRNA) encoding a therapeutic polypeptide in combination with one or more additional therapies to a subject in need thereof. In a specific embodiment, presented herein are combination therapies for the treatment of a disease or disorder that would benefit from increased expression of a therapeutic polypeptide which involve the administration of an effective amount of a polynucleotide (e.g., mRNA) encoding a therapeutic polypeptide in combination with an effective amount of another therapy to a subject in need thereof.

The combination therapies provided herein involve administrating to a subject in need thereof a polynucleotide or a pharmaceutical composition thereof in combination with conventional, or known, therapies for a disease or disorder that would benefit from increased expression of a therapeutic polypeptide. Other therapies for a disease or disorder that would benefit from increased expression of a therapeutic polypeptide or a condition associated therewith are aimed at controlling or relieving symptoms. Accordingly, in some embodiments, the combination therapies provided herein involve administrating to a subject to in need thereof a pain reliever, a medication for epileptic seizures, or other therapy aimed at alleviating or controlling symptoms associated with the disease or disorder that would benefit from increased expression of a therapeutic polypeptide or a condition associated therewith.

In some embodiments, the methods for treating a disease or disorder that would benefit from increased expression of a therapeutic polypeptide provided herein comprise administering a polynucleotide (e.g., mRNA) encoding a therapeutic polypeptide as a single agent for a period of time prior to administering the polynucleotide in combination with an additional therapy. In some embodiments, the methods for treating a disease or disorder that would benefit from increased expression of a therapeutic polypeptide provided herein comprise administering an additional therapy alone for a period of time prior to administering a polynucleotide (e.g., mRNA) encoding a therapeutic polypeptide in combination with the additional therapy.

In some embodiments, the administration of a polynucleotide (e.g., mRNA) encoding a therapeutic polypeptide and one or more additional therapies in accordance with the methods presented herein have an additive effect relative the administration of the polynucleotide or said one or more additional therapies alone. In some embodiments, the administration of a polynucleotide (e.g., mRNA) encoding a therapeutic polypeptide and one or more additional therapies in accordance with the methods presented herein have a synergistic effect relative to the administration of the polynucleotide or said one or more additional therapies alone.

The combination of a polynucleotide (e.g., mRNA) encoding a therapeutic polypeptide and one or more additional therapies is administered to a subject in the same pharmaceutical composition. Alternatively, a polynucleotide (e.g., mRNA) encoding a therapeutic polypeptide and one or more additional therapies is administered concurrently to a subject in separate pharmaceutical compositions. A polynucleotide (e.g., mRNA) encoding a therapeutic polypeptide and one or more additional therapies is administered sequentially to a subject in separate pharmaceutical compositions. A polynucleotide (e.g., mRNA) encoding a therapeutic polypeptide and one or more additional therapies is administered to a subject by the same or different routes of administration.

In some embodiments, the combination therapies provided herein involve administering to a subject in need thereof a polynucleotide (e.g., mRNA) encoding a therapeutic polypeptide or a pharmaceutical composition thereof in combination with one or more of the following: a nutritional supplement, and an antibiotic. In some embodiments, the combination therapies provided herein involve administering to a subject in need thereof a polynucleotide (e.g., mRNA) encoding a therapeutic polypeptide or a pharmaceutical composition thereof in combination with an organ transplant (e.g., a kidney, liver or kidney and liver transplant).

### Clinical Objectives and Endpoints

In some embodiments, maintenance of a desired effect is determined by analyzing a biological sample (e.g., biopsy) at least once during a dosing interval. In some embodiments, maintenance of a desired effect is determined by analyzing a biological sample (e.g., biopsy) at regular intervals during a dosing interval. In some embodiments, maintenance of a desired effect is determined by analyzing a biological sample (e.g., biopsy) before a subsequent dose is administered.

In some embodiments, dosing occurs until a positive therapeutic outcome is achieved. In some embodiments, dosing of a composition comprising mRNAs encoding polypeptides of interest will occur indefinitely, or until a positive therapeutic outcome is achieved. In some embodiments, the dosing interval remains consistent. In some embodiments, the dosing interval changes as needed based on a clinician's assessment.

In some aspects, efficacy of a polynucleotide for treating a disease or disorder that would benefit from increased expression of a therapeutic polypeptide is assessed by determining the effects of the polynucleotide on reduction of a biomarker of the therapeutic polypeptide. The efficacy of a polynucleotide for treating a disease or disorder that would benefit from increased expression of a therapeutic polypeptide may also be assessed by: (i) determining the effect on levels of one or more biomarker of the therapeutic polypeptides; (ii) determining the effects on enzyme activity in cultured fibroblasts and lymphocytes from subjects with the disease or disorder that would benefit from increased expression of a therapeutic polypeptide, (iii) evaluating the safety profile of the polynucleotide; (iv) evaluating compliance with treatment with the polynucleotide; and (v) determining the polynucleotide's plasma exposure over time.

A primary clinical endpoint for efficacy of a polynucleotide for treating a disease or disorder that would benefit from increased expression of a therapeutic polypeptide includes a reduction in a biomarker of the therapeutic polypeptide. Other clinical endpoints for the efficacy of a polynucleotide for treating a disease or disorder that would benefit from increased expression of a therapeutic polypeptide may include a reduction in one or more biomarker of the therapeutic polypeptide and pharmacokinetic parameters, e.g., time to maximum plasma concentration (Tₘₐₓ), Cₘₐₓ, AUC, terminal elimination half-life (t_{1/2}) based on a polynucleotide's plasma concentrations as assessed by a validated bioanalytical method.

### Definitions

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular.

Activity: As used herein, the term “activity” refers to an activity of a polypeptide (e.g., an enzyme) encoded by an mRNA of the disclosure. As is generally known by one skilled in the art, enzymes are macromolecular biological catalysts that accelerate biochemical reactions. The molecule upon which a enzyme acts is called a substrate and the enzyme converts the substrates into different molecules known as products. In some embodiments, the activity of a polypeptide is the conversion of a substrate into a product. The activity of a polypeptide is determined by any suitable method known in the art. In some embodiments, the activity of a polypeptide is determined by measuring the enzymatic activity or specific enzymatic activity of the polypeptide. In some embodiments, the activity of a polypeptide is determined by detecting the presence or determining an amount of product formed by the polypeptide in a sample or a subject.

Enzymatic activity is a measure of a quantity of active enzyme and is expressed in enzyme units (“U”) per volume, mass or weight of a sample or total protein within a sample. 1 enzyme unit (U) is defined as the amount of enzyme that catalyzes the conversion of one nanomole of substrate per hour (nmol/hr) under the specified conditions of an enzyme assay. The specified conditions are typically the optimum conditions that yield the maximal substrate conversion rate for a particular enzyme, and may include, but not be limited to, optimal temperature, pH and substrate concentration. In exemplary embodiments, the activity of a polypeptide (e.g., an enzyme) is described in terms of units (U) per milliliter (mL) of fluid (e.g., bodily fluid, e.g., serum, plasma, urine and the like) or is described in terms of units (U) per weight of tissue or per weight of protein (e.g., total protein) within a sample. In some embodiments, the activity of a polypeptide encoded by an mRNA of the disclosure is described in terms of U/mL plasma or U/mg protein (tissue). In some embodiments, the activity of a polypeptide encoded by an mRNA of the disclosure is described in terms of U/mL cell lysate or U/mL of tissue homogenate.

Some aspects of the disclosure feature measurement, determination and/or monitoring of the activity of a polypeptide encoded by an mRNA, such as those described herein, in a sample or a subject, for example, in animals (e.g., rodents, primates, and the like) or in human subjects. In some embodiments, an activity of a polypeptide encoded by an mRNA of the disclosure is measured, determined, or monitored by any art-recognized method for determining enzymatic activity in biological samples. In some embodiments, an activity of a polypeptide encoded by an mRNA of the disclosure is measured, determined, or monitored by any art-recognized method for detecting the presence or determining an amount of product formed by the polypeptide in biological samples. The skilled artisan will appreciate that the mRNAs provided by the disclosure can be characterized or determined by measuring the activity of a polypeptide (e.g., enzyme) encoded by the mRNA in a sample or in samples taken from a subject (e.g., from a preclinical test subject (rodent, primate, etc.) or from a clinical subject (human).

Administering: As used herein, “administering” refers to a method of delivering a composition to a subject or patient. A method of administration may be selected to target delivery (e.g., to specifically deliver) to a specific region or system of a body. For example, an administration may be parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g. by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter.

Altering: As used herein, “altered” or “altering” refers to a change in the chemical composition, structure, or functionality of an mRNA. In some embodiments, an mRNA of the disclosure is altered to achieve increased potency or increased stability relative to an unaltered mRNA. For example, in some embodiments, an mRNA is altered to increase potency and/or stability by reducing the sensitivity of the mRNA to endonuclease-mediated cleavage. In some embodiments, altering an mRNA to reduce sensitivity to endonuclease-mediated cleavage comprises substitution, insertion, or deletion of the mRNA sequence that is sensitive to endonuclease cleavage. In some embodiments, altering an mRNA to remove one or more endonuclease cleavage site(s) comprises substitution of the endonuclease sensitive sequence with one or more degenerate codons that are less susceptible to endonuclease activity. In another embodiment, altering an mRNA to remove one or more endonuclease cleavage site(s) comprises substitution of the endonuclease sensitive sequence with at least one or more chemically modified nucleotides that are less susceptible to endonuclease activity. In some embodiments, an mRNA is altered to have increased half-life following contacting the mRNA with a cell.

Approximately, about: As used herein, the terms “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Base Composition: As used herein, the term “base composition” refers to the proportion of the total bases of a nucleic acid consisting of guanine+cytosine or thymine (or uracil)+adenine nucleotides.

Base Pair: As used herein, the term “base pair” refers to two nucleobases on opposite complementary nucleic acid strands that interact via the formation of specific hydrogen bonds. As used herein, the term “Watson-Crick base pairing”, used interchangeably with “complementary base pairing”, refers to a set of base pairing rules, wherein a purine always binds with a pyrimidine such that the nucleobase adenine (A) forms a complementary base pair with thymine (T) and guanine (G) forms a complementary base pair with cytosine (C) in DNA molecules. In RNA molecules, thymine is replaced by uracil (U), which, similar to thymine (T), forms a complementary base pair with adenine (A). The complementary base pairs are bound together by hydrogen bonds and the number of hydrogen bonds differs between base pairs. As in known in the art, guanine (G)-cytosine (C) base pairs are bound by three (3) hydrogen bonds and adenine (A)-thymine (T) or uracil (U) base pairs are bound by two (2) hydrogen bonds. Base pairing interactions that do not follow these rules can occur in natural, non-natural, and synthetic nucleic acids and are referred to herein as “non-Watson-Crick base pairing” or alternatively “non-complementary base pairing”.

Biomarker: As used herein, the term “biomarker” (alternatively “response biomarker”) refers to a substance that is detected and/or measured in a sample or subject as an indicator of an activity of a polypeptide. For example, in some aspects, a biomarker is a product formed by the activity of a polypeptide encoded by an mRNA provided by the disclosure. In other aspects, a biomarker is a receptor whose expression and/or activity changes in response to the activity of a polypeptide encoded by an mRNA of the disclosure. In some aspects, the activity of a polypeptide is characterized or determined by measuring the level of an appropriate biomarker in sample(s) taken from a subject. The term “level of a biomarker” as used herein, preferably means the mass, weight or concentration of a biomarker, for example, a product formed by the activity of a polypeptide encoded by an mRNA, described herein, within a sample or a subject. It will be understood by the skilled artisan that in certain embodiments the sample may be subjected, e.g. to a step of substance purification, precipitation, separation, e.g. centrifugation and/or HPLC and subsequently subjected to a step of determining the level of the biomarker, e.g. using mass spectrometric analysis. In exemplary embodiments, LC-MS can be used as a means for determining the level of a biomarker according to the invention.

Cap structure or 5′ cap structure: As used herein, the terms “cap structure”, “5′ cap structure” and “5′cap” refer to a non-extendible dinucleotide that facilitates translation or localization, and/or prevents degradation of an RNA transcript when incorporated at the 5′ end of an RNA transcript, wherein the cap structure can be a natural cap, a derivative of a natural cap, or any chemical group that protects the 5′end of an RNA from degradation and/or is essential for translation initiation. In nature, the modified base 7-methylguanosine is joined in the opposite orientation, 5′ to 5′ rather than 5′ to 3′, to the rest of the molecule via three phosphate groups (i.e., P1-guanosine-5′-yl P3-7-methylguanosine-5′-yl triphosphate (m⁷G5′ppp5′G)). In some embodiments, the mRNA provided herein comprises a “cap analog”, which refers to a structural derivative of an RNA cap that may differ by as little as a single element. In some embodiments, the mRNA provided herein comprises a “mCAP”, which refers to a dinucleotide cap with the N7 position of the guanosine having a methyl group. The structure can be represented as m⁷G(5′)ppp(g′)G, through a triphosphate, a tetraphosphate or a pentaphosphate group can join the two nucleotides.

C-content: As used herein, the term “C-content” refers to the percentage of nucleobases in a polynucleotide (e.g., mRNA), or a portion thereof (e.g., an RNA element), that are cytosine (C) nucleobases, or derivatives or analogs thereof, (from a total number of possible nucleobases, including guanine (G), adenine (A) and thymine (T) or uracil (U), and derivatives or analogs thereof, in DNA and in RNA). The term “C-content” refers to all, or to a portion, of a polynucleotide, including, but not limited to, a gene, a non-coding region, a 5′ or 3′ UTR, an open reading frame, an RNA element, a sequence motif, or any discrete sequence, fragment, or segment thereof. In some aspects, the C-content of a C-rich RNA element comprises at least 50% or greater cytosine nucleobases, or derivatives or analogs thereof, and less than 10% guanosine nucleobases, or derivatives or analogs thereof. In some aspects, the C-content of a C-rich RNA element comprises at least 50% or greater cytosine nucleobases, or derivatives or analogs thereof, and less than 5% guanosine nucleobases, or derivatives or analogs thereof. In some aspects, the C-content of a C-rich RNA element comprises at least 50% or greater cytosine nucleobases, or derivatives or analogs thereof, with the remaining content comprising adenosine nucleobases, or derivatives or analogs thereof. In some aspects, the C-content of a C-rich RNA element comprises at least 50% or greater cytosine nucleobases, or derivatives or analogs thereof, with the remaining content comprising adenosine nucleobases and uracil nucleobases, or derivatives or analogs thereof (e.g., pseudouridine, N1-methyl pseudouridine, 5-methoxyuridine) and no guanosine nucleobases. In some aspects, the C-content of a C-rich RNA element comprises at least 50% or greater cytosine nucleobases, or derivatives or analogs thereof, with the remaining content comprising preferentially adenosine>uracilguanosine (A>U>>G) nucleobases, or derivatives or analogs thereof (e.g., pseudouridine, N1-methyl pseudouridine, 5-methoxyuridine). In some aspects, the C-content of a C-rich RNA element comprises at least 50% or greater cytosine nucleobases, or derivatives or analogs thereof, with the remaining content comprising preferentially adenosine (15-45%), uracil (5-10%) and guanosine (5%-10%) nucleobases, or derivatives or analogs thereof (e.g., pseudouridine, N1-methyl pseudouridine, 5-methoxyuridine).

Conjugated: As used herein, the term “conjugated,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. In some embodiments, two or more moieties may be conjugated by direct covalent chemical bonding. In other embodiments, two or more moieties may be conjugated by ionic bonding or hydrogen bonding.

Contacting: As used herein, the term “contacting” means establishing a physical connection between two or more entities. For example, contacting a cell with an mRNA or a lipid nanoparticle composition means that the cell and mRNA or lipid nanoparticle are made to share a physical connection. Methods of contacting cells with external entities both in vivo, in vitro, and ex vivo are well known in the biological arts. In exemplary embodiments of the disclosure, the step of contacting a mammalian cell with a composition (e.g., an isolated mRNA, nanoparticle, or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a lipid nanoparticle or an isolated mRNA) and a cell may be contacted, for example, by adding the composition to the culture medium of the cell and may involve or result in transfection. Moreover, more than one cell may be contacted by a nanoparticle composition.

C-rich: As used herein, the term “C-rich” refers to the nucleobase composition of a polynucleotide (e.g., mRNA), or any portion thereof (e.g., a C-rich RNA element), comprising cytosine (C) nucleobases, or derivatives or analogs thereof, wherein the C-content is at least 50% or greater and is located proximal to the 5′ end of the mRNA (e.g., proximal to the 5′ cap). In some aspects, the term C-rich (e.g., a C-rich RNA element) comprises at least 55% or greater, at least 60% or greater, at least 65% or greater, at least 70% or greater, at least 75% or greater, at least 80% or greater, at least 85% or greater, at least 90% or greater, about 90%, about 91%, about 92%, about 93%, about 94%, or about 95% cytosine nucleobases, or derivatives or analogs thereof. In some embodiments that C-rich element comprises at least 95%, 96%, 97%, 98%, 99% or 100% cytosine nucleobases, or derivatives or analogs thereof. In some embodiments, the C-rich RNA element is about 15 nucleotides and comprises at least 90% or at 100% cytosine nucleobases, or derivatives or analogs thereof. The term “C-rich” refers to all, or to a portion, of a polynucleotide, including, but not limited to, a gene, a non-coding region, a 5′ UTR, a 3′ UTR, an open reading frame, an RNA element, a sequence motif, or any discrete sequence, fragment, or segment thereof which comprises at least 50% or greater C-content. In some aspects, C-rich polynucleotides, or any portions thereof, are exclusively comprised of cytosine (C) nucleobases. In some aspects, a C-rich polynucleotide comprises a C-rich RNA element comprising a sequence of linked nucleotides, or derivatives or analogs thereof, wherein each nucleotide comprises a nucleobase selected from the group consisting of: adenine, guanine, thymine, uracil, and cytosine, linked in any order. In some aspects, the C-rich RNA element comprises about 3-20 nucleotides. In some aspects, the C-rich RNA element is located within a 5′UTR of an mRNA and is located proximal to the 5′ end of the mRNA (e.g., proximal to the 5′ cap). In some aspects, the C-rich RNA element is located within a 5′UTR of an mRNA and is located adjacent to or within about 1-6 or about 1-10 nucleotides downstream of the 5′ end of the mRNA (e.g., adjacent to or within about 1-6 or about 1-10 nucleotides downstream of the 5′ cap). In some aspects, the C-rich RNA element is located within a 5′UTR of an mRNA and is located about 1-20, about 2-15, about 3-10, about 4-8, or about 6 nucleotides downstream of the 5′ cap in the 5′ UTR.

Determining the level: As used herein, the term “determining the level” of a substance (e.g., biomarker) refers to methods to quantify an amount of the substance in a sample, for example, from a subject (e.g., a bodily fluid; e.g., blood, lymph, serum, plasma, urine, etc.) or in a tissue of the subject (e.g., liver, heart, spleen kidney, etc.).

Encapsulate: As used herein, the term “encapsulate” means to enclose, surround, or encase. In some embodiments, a compound, an mRNA, or other composition may be fully encapsulated, partially encapsulated, or substantially encapsulated. For example, in some embodiments, an mRNA of the disclosure may be encapsulated in a lipid nanoparticle, e.g., a liposome.

Effective amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent. In some embodiments, a therapeutically effective amount is an amount of an agent to be delivered (e.g., nucleic acid, drug, therapeutic agent, diagnostic agent or prophylactic agent) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Endonuclease: As used herein, “endonuclease” refers to a cellular enzyme that cleaves the phosphodiester bond with a polynucleotide chain. An endonuclease differs from an exonuclease that cleaves terminal phosphodiester bonds of polynucleotides. In some embodiments, an endonuclease refers to an enzyme that cleaves a phosphodiester bond of DNA, of RNA or of both DNA and RNA. In some embodiments, an endonuclease refers to an enzyme that cleaves the phosphodiester bond within RNA. In some embodiments, an endonuclease cleaves a phosphodiester bond within an RNA to generate a 5′ RNA product and a 3′ RNA product, wherein the 5′ RNA product comprises a 3′ hydroxyl terminus and the 3′ RNA product comprises a 5′ phosphate group. In some embodiments, an endonuclease cleaves a phosphodiester bond within an RNA to generate a 5′ RNA product and a 3′ RNA product, wherein the 5′ RNA product comprises a 2′3′ cyclic phosphate and the 3′ RNA product comprises a 5′ hydroxyl group. In some embodiments, an endonuclease cleaves a phosphodiester bond non-specifically, wherein cleavage occurs at any site within an RNA regardless of the surrounding RNA sequence or structure. In some embodiments, an endonuclease cleaves a phosphodiester bond specifically, wherein cleavage occurs at specific sites within an RNA that is dependent upon the surrounding RNA sequence or structure.

Endonuclease activity: As used herein, “endonuclease activity” refers to the efficiency of enzymatic cleavage of a polynucleotide by one or more endonucleases. The efficiency of enzymatic cleavage of a polynucleotide is determined by measuring the rate of cleavage of a polynucleotide as a function of concentration of the polynucleotide according to methods known in the art. In some embodiments, the efficiency of enzymatic cleavage is measured after contacting the polynucleotide with a recombinant endonuclease, after contacting the polynucleotide with a mixture of recombinant endonucleases, after contacting the polynucleotide with cellular lysate, or after contacting the polynucleotide with a cell. In some embodiments, endonuclease activity refers to the efficiency of enzymatic cleavage of a polynucleotide at a single site within the polynucleotide. In some embodiments, endonuclease activity refers to the efficiency of enzymatic cleavage of a polynucleotide at more than one sites within the polynucleotide.

In some embodiments, the efficiency of enzymatic cleavage of a polynucleotide by one or more endonucleases is measured for an altered polynucleotide compared to an equivalent unaltered polynucleotide counterpart. Increased resistance to endonuclease activity for an altered polynucleotide is defined as decreased efficiency of enzymatic cleavage compared to an unaltered polynucleotide counterpart.

Increases Potency: As used herein, the term “increases potency” “increase potency of an mRNA” refers to the need to administer less of the mRNA to achieve the same functional effect as a less potent mRNA, as a result of, e.g., an increase in functional protein translated from an mRNA (e.g., an endonuclease-resistant mRNA). In some embodiments, an increase in potency occurs owing to an increase in the endonuclease resistance of an mRNA, resulting in an increase in total protein output translated from the mRNA. In some embodiments, an increase in potency occurs due to an increase in the half-life of an mRNA, resulting in an increase in total protein output translated from the mRNA. In some embodiments, an increase in half-life of an mRNA occurs due to (i) reduced sensitivity to an endonuclease cleavage event, (ii) reduced sensitivity to an exonuclease cleavage event, or (iii) reduced stop codon read through. In some embodiments, an increase in half-life increases the number of polypeptide molecules translated per mRNA.

Increases Stability: As used herein, the term “increases stability” or “increases stability of an mRNA” refers to an increase in the ability of the mRNA to resist, reduce or inhibit degradation, and/or increase or improve mRNA half-life. mRNA degradation can occur through physical (e.g., shear or UV radiation), chemical (e.g., hydrolysis), or enzymatic (e.g. nuclease activity) means. Degradation of an mRNA occurs both prior to contacting the mRNA with a cell or after contacting the mRNA with a cell. Upon contacting an mRNA with a cell, cellular machinery induces mRNA degradation, e.g., by enzymatic cleavage of the mRNA. In some aspects, the disclosure relates to altering an mRNA to reduce susceptibility of the mRNA to enzyme-mediated degradation (e.g., exonuclease or endonuclease-mediated degradation) either prior to or following contacting the mRNA with a cell. Reducing the rate of enzymatic degradation of an mRNA in a cell results in increased mRNA half-life or stability. In some embodiments, increased stability of an altered mRNA is measured relative to an unaltered mRNA counterpart (e.g., the starting mRNA prior to altering endonuclease sensitive motifs). In some embodiments, the unaltered mRNA counterpart is endonuclease sensitive, and altering the mRNA yields a stabilized mRNA, wherein the endonuclease resistant mRNA has an increased half-life relative to the endonuclease sensitive unaltered mRNA.

Expression: As used herein, “expression” refers to translation of an RNA (e.g., an mRNA) into a polypeptide or protein.

Expression level: As used herein, the term “expression level” preferably means an amount (weight or mass) or concentration of a polypeptide or a protein translated from an RNA (e.g., an mRNA) within a sample or a subject. It will be understood by the skilled artisan that in certain embodiments the sample may be subjected, e.g. to a step of purification, precipitation, separation, e.g. centrifugation and/or HPLC, and subsequently subjected to a step of determining an expression level, e.g., using mass and/or spectrometric analysis. In exemplary embodiments, enzyme-linked immunosorbent assay (ELISA) can be used to determine protein expression levels. In other exemplary embodiments, protein purification, separation and LC-MS can be used as a means for determining the level of a protein according to the invention.

The skilled artisan will appreciate that the mRNAs provided by the disclosure can be characterized or determined by measuring the level of expression of an encoded polypeptide (e.g., enzyme) in a sample or in samples taken from a subject (e.g., from a preclinical test subject (rodent, primate, etc.) or from a clinical subject (human). The terms “Elevated expression,” “elevated expression levels,” or “elevated levels” refers to an increase in the translation, and thereby amount, of a polypeptide encoded by an mRNA, described herein, within a sample or subject contacted with or administered the mRNA relative to a control sample or control subject, such as a sample or subject that was not contacted with or administered the mRNA or was contacted with or administered a control mRNA or reference mRNA. In some embodiments, the elevated expression in a sample refers to an increase in the amount of the polypeptide of about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% relative to the amount of the peptide in a control sample, as determined by techniques known in the art (e.g., Western blot). “Reduced expression,” “reduced expression levels,” or “reduced levels” refers to a decrease in translation, and thereby amount, of a polypeptide encoded by an mRNA described herein, within a sample or subject contacted with or administered the mRNA relative to a control sample or control subject, such as a sample or subject that was not contacted with or administered the mRNA or was contacted with or administered a control mRNA or reference mRNA. In some embodiments, reduced expression is little or no expression. In some embodiments, the reduced expression of a substance (e.g., a protein or a biomarker) in a sample refers to a decrease in the amount of the polypeptide of about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% relative to the amount of the substance in a control sample, as determined by techniques known in the art (e.g, Western blot).

Fragment: A “fragment,” as used herein, refers to a portion. For example, fragments of proteins may include polypeptides obtained by digesting full-length protein isolated from cultured cells or obtained through recombinant DNA techniques.

GC-content: As used herein, the term “GC-content” refers to the percentage of nucleobases in a polynucleotide (e.g., mRNA), or a portion thereof (e.g., an RNA element), that are either guanine (G) and cytosine (C) nucleobases, or derivatives or analogs thereof, (from a total number of possible nucleobases, including adenine (A) and thymine (T) or uracil (U), and derivatives or analogs thereof, in DNA and in RNA (e.g., pseudouridine, N1-methyl pseudouridine, 5-methoxyuridine)). The term “GC-content” refers to all, or to a portion, of a polynucleotide, including, but not limited to, a gene, a non-coding region, a 5′ or 3′ UTR, an open reading frame, an RNA element, a sequence motif, or any discrete sequence, fragment, or segment thereof.

GC-rich: As used herein, the term “GC-rich” refers to the nucleobase composition of a polynucleotide (e.g., mRNA), or any portion thereof (e.g., an RNA element), comprising guanine (G) and/or cytosine (C) nucleobases, or derivatives or analogs thereof, wherein the GC-content is at least 50% or greater. The term “GC-rich” refers to all, or to a portion, of a polynucleotide, including, but not limited to, a gene, a non-coding region, a 5′ UTR, a 3′ UTR, an open reading frame, an RNA element, a sequence motif, or any discrete sequence, fragment, or segment thereof which comprises at least 50% or greater GC-content. In some aspects, the term GC-rich (e.g., a GC-rich RNA element) comprises at least 55% or greater, at least 60% or greater, at least 65% or greater, at least 70% or greater, at least 75% or greater, at least 80% or greater, at least 85% or greater, at least 90% or greater, or at least 95%, 96%, 97%, 98%, 99% or 100% guanosine and cytosine nucleobases, or derivatives or analogs thereof. In some embodiments of the disclosure, GC-rich polynucleotides, or any portions thereof, are exclusively comprised of guanine (G) and/or cytosine (C) nucleobases.

Heterologous: As used herein, “heterologous” indicates that a sequence (e.g., an amino acid sequence or the nucleic acid that encodes an amino acid sequence) is not normally present in a given polypeptide or nucleic acid. For example, an amino acid sequence that corresponds to a domain or motif of one protein may be heterologous to a second protein.

Identity: As used herein, the term “identity” refers to the overall relatedness between polymeric molecules, e.g., between nucleic acid molecules (e.g., DNA molecules and/or RNA molecules) and/or between polypeptide molecules. Calculation of the percent identity of two mRNA sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux et al., Nucleic Acids Research, 12(1): 387,1984, BLASTP, BLASTN, and FASTA, Altschul, S. F. et al., J. Molec. Biol., 215, 403, 1990.

Initiation Codon: As used herein, the term “initiation codon”, used interchangeably with the term “start codon”, refers to the first codon of an open reading frame that is translated by the ribosome and is comprised of a triplet of linked adenine-uracil-guanine nucleobases. The initiation codon is depicted by the first letter codes of adenine (A), uracil (U), and guanine (G) and is often written simply as “AUG”. Although natural mRNAs may use codons other than AUG as the initiation codon, which are referred to herein as “alternative initiation codons”, the initiation codons of polynucleotides described herein use the AUG codon. During the process of translation initiation, the sequence comprising the initiation codon is recognized via complementary base-pairing to the anticodon of an initiator tRNA (Met-tRNAᵢ^{Met}) bound by the ribosome. Open reading frames may contain more than one AUG initiation codon, which are referred to herein as “alternate initiation codons”.

The initiation codon plays a critical role in translation initiation. The initiation codon is the first codon of an open reading frame that is translated by the ribosome. Typically, the initiation codon comprises the nucleotide triplet AUG, however, in some instances translation initiation can occur at other codons comprised of distinct nucleotides. The initiation of translation in eukaryotes is a multistep biochemical process that involves numerous protein-protein, protein-RNA, and RNA-RNA interactions between messenger RNA molecules (mRNAs), the 40S ribosomal subunit, other components of the translation machinery (e.g., eukaryotic initiation factors; eIFs). The current model of mRNA translation initiation postulates that the pre-initiation complex (alternatively “43S pre-initiation complex”: abbreviated as “PIC”) translocates from the site of recruitment on the mRNA (typically the 5′ cap) to the initiation codon by scanning nucleotides in a 5′ to 3′ direction until the first AUG codon that resides within a specific translation-promotive nucleotide context (the Kozak sequence) is encountered (Kozak (1989) J Cell Biol 108:229-241). Scanning by the PIC ends upon complementary base-pairing between nucleotides comprising the anticodon of the initiator Met-tRNAᵢ^{Met} transfer RNA and nucleotides comprising the initiation codon of the mRNA. Productive base-pairing between the AUG codon and the Met-tRNAᵢ^{Met} anticodon elicits a series of structural and biochemical events that culminate in the joining of the large 60S ribosomal subunit to the PIC to form an active ribosome that is competent for translation elongation.

Insertion: As used herein, an “insertion” or an “addition” refers to a change in an amino acid or nucleotide sequence resulting in the addition of one or more amino acid residues or nucleotides, respectively, to a molecule as compared to a reference sequence, for example, the sequence found in a naturally-occurring molecule. For example, an amino acid sequence of a heterologous polypeptide (e.g., a BH3 domain) may be inserted into a scaffold polypeptide (e.g. a SteA scaffold polypeptide) at a site that is amenable to insertion. In some embodiments, an insertion may be a replacement, for example, if an amino acid sequence that forms a loop of a scaffold polypeptide (e.g., loop 1 or loop 2 of SteA or a SteA derivative) is replaced by an amino acid sequence of a heterologous polypeptide.

Insertion Site: As used herein, an “insertion site” is a position or region of a scaffold polypeptide that is amenable to insertion of an amino acid sequence of a heterologous polypeptide. It is to be understood that an insertion site also may refer to the position or region of the mRNA that encodes the polypeptide (e.g., a codon of an mRNA that codes for a given amino acid in the scaffold polypeptide). In some embodiments, insertion of an amino acid sequence of a heterologous polypeptide into a scaffold polypeptide has little to no effect on the stability (e.g., conformational stability), expression level, or overall secondary structure of the scaffold polypeptide.

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Kozak Sequence: The term “Kozak sequence” (also referred to as “Kozak consensus sequence”) refers to a translation initiation enhancer element to enhance expression of a gene or open reading frame, and which in eukaryotes, is located in the 5′ UTR. The Kozak consensus sequence was originally defined as the sequence GCCRCC, where R=a purine, following an analysis of the effects of single mutations surrounding the initiation codon (AUG) on translation of the preproinsulin gene (Kozak (1986) Cell 44:283-292). Polynucleotides disclosed herein comprise a Kozak consensus sequence, or a derivative or modification thereof. (Examples of translational enhancer compositions and methods of use thereof, see U.S. Pat. No. 5,807,707 to Andrews et al., incorporated herein by reference in its entirety; U.S. Pat. No. 5,723,332 to Chernajovsky, incorporated herein by reference in its entirety; U.S. Pat. No. 5,891,665 to Wilson, incorporated herein by reference in its entirety.)

Kozak-like sequence: As used herein, the term “Kozak-like sequence” refers to a sequence similar to the Kozak sequence described supra, comprising an adenine or guanine three nucleotides upstream of the AUG start codon. In some embodiments, the Kozak-like sequence is gcc(X)ccAUG, wherein X is A or G, and wherein the lower case letters indicate bases that are weakly preferred.

Leaky scanning: As used herein, the term “leaky scanning” refers to a biological phenomenon whereby the pre-initiation complex (PIC) bypasses the initiation codon of an mRNA and instead continues scanning downstream until an alternate or alternative initiation codon is recognized. Depending on the frequency of occurrence, the bypass of the initiation codon by the PIC can result in a decrease in translation efficiency. Furthermore, translation from this downstream AUG codon can occur, which will result in the production of an undesired, aberrant translation product that may not be capable of eliciting the desired therapeutic response. In some cases, the aberrant translation product may in fact cause a deleterious response (Kracht et al., (2017) Nat Med 23(4):501-507)

Liposome: As used herein, by “liposome” is meant a structure including a lipid-containing membrane enclosing an aqueous interior. Liposomes may have one or more lipid membranes. Liposomes include single-layered liposomes (also known in the art as unilamellar liposomes) and multi-layered liposomes (also known in the art as multilamellar liposomes).

Linker: As used herein, a “linker” (including a subunit linker, and a heterologous polypeptide linker as referred to herein) refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker can be of sufficient length as to not interfere with incorporation into a nucleic acid sequence. The linker can be used for any useful purpose, such as to form polynucleotide multimers (e.g., through linkage of two or more chimeric polynucleotides molecules or IVT polynucleotides) or polynucleotides conjugates, as well as to administer a payload, as described herein. Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers and derivatives thereof, Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond can be cleaved for example by the use of tris(2-carboxyethyl)phosphine (TCEP), or other reducing agents, and/or photolysis, as well as an ester bond can be cleaved for example by acidic or basic hydrolysis. mRNA: As used herein, an “mRNA” refers to a messenger ribonucleic acid. An mRNA may be naturally or non-naturally occurring. For example, an mRNA may include modified and/or non-naturally occurring components such as one or more nucleobases, nucleosides, nucleotides, or linkers.

An mRNA may include a cap structure, a chain terminating nucleoside, a stem loop, a polyA sequence, and/or a polyadenylation signal. An mRNA may have a nucleotide sequence encoding a polypeptide.

Translation of an mRNA, for example, in vivo translation of an mRNA inside a mammalian cell, may produce a polypeptide. Traditionally, the basic components of an mRNA molecule include at least a coding region, a 5′-untranslated region (5′-UTR), a 3′UTR, a 5′ cap and a polyA sequence.

microRNA (miRNA): As used herein, a “microRNA (miRNA)” is a small non-coding RNA molecule which may function in post-transcriptional regulation of gene expression (e.g., by RNA silencing, such as by cleavage of the mRNA, destabilization of the mRNA by shortening its polyA tail, and/or by interfering with the efficiency of translation of the mRNA into a polypeptide by a ribosome). A mature miRNA is typically about 22 nucleotides long.

microRNA (miRNA) binding site: As used herein, a “microRNA (miRNA) binding site” refers to a miRNA target site or a miRNA recognition site, or any nucleotide sequence to which a miRNA binds or associates. In some embodiments, a miRNA binding site represents a nucleotide location or region of an mRNA to which at least the “seed” region of a miRNA binds. It should be understood that “binding” may follow traditional Watson-Crick hybridization rules or may reflect any stable association of the miRNA with the target sequence at or adjacent to the microRNA site.

miRNA seed: As used herein, a “seed” region of a miRNA refers to a sequence in the region of positions 2-8 of a mature miRNA, which typically has perfect Watson-Crick complementarity to the miRNA binding site. A miRNA seed may include positions 2-8 or 2-7 of a mature miRNA. In some embodiments, a miRNA seed may comprise 7 nucleotides (e.g., nucleotides 2-8 of a mature miRNA), wherein the seed-complementary site in the corresponding miRNA binding site is flanked by an adenine (A) opposed to miRNA position 1. In some embodiments, a miRNA seed may comprise 6 nucleotides (e.g., nucleotides 2-7 of a mature miRNA), wherein the seed-complementary site in the corresponding miRNA binding site is flanked by an adenine (A) opposed to miRNA position 1. When referring to a miRNA binding site, an miRNA seed sequence is to be understood as having complementarity (e.g., partial, substantial, or complete complementarity) with the seed sequence of the miRNA that binds to the miRNA binding site.

Mitochondrial targeting sequence: As used herein, the term “mitochondrial targeting sequence” (abbreviated MTS), alternatively “mitochondrial targeting signal” or “presequence” refers to any peptide that directs, localizes, translocates, sorts, or otherwise delivers a polypeptide to the mitochondrion of a cell. MTSs are known to one of ordinary skill in the art. The MTS is an approximately 10-90 amino acid long peptide that directs a newly synthesized mitochondrial protein to the mitochondria. It is found at the N-terminus end and consists of an alternating pattern of hydrophobic and positively charged amino acids to form an amphipathic helix. Mitochondrial targeting signals can contain additional signals that subsequently target the protein to different subcompartments of the mitochondria, such as the mitochondrial matrix.

Modified: As used herein “modified” refers to a changed state or structure of a molecule of the disclosure. Molecules may be modified in many ways including chemically, structurally, and functionally. In one embodiment, the mRNA molecules of the present disclosure are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

Nanoparticle: As used herein, “nanoparticle” refers to a particle having any one structural feature on a scale of less than about 1000 nm that exhibits novel properties as compared to a bulk sample of the same material. Routinely, nanoparticles have any one structural feature on a scale of less than about 500 nm, less than about 200 nm, or about 100 nm. Also routinely, nanoparticles have any one structural feature on a scale of from about 50 nm to about 500 nm, from about 50 nm to about 200 nm or from about 70 to about 120 mn. In exemplary embodiments, a nanoparticle is a particle having one or more dimensions of the order of about 1-1000 nm. In other exemplary embodiments, a nanoparticle is a particle having one or more dimensions of the order of about 10-500 nm. In other exemplary embodiments, a nanoparticle is a particle having one or more dimensions of the order of about 50-200 nm. A spherical nanoparticle would have a diameter, for example, of between about 50-100 or 70-120 nanometers. A nanoparticle most often behaves as a unit in terms of its transport and properties. It is noted that novel properties that differentiate nanoparticles from the corresponding bulk material typically develop at a size scale of under 1000 nm, or at a size of about 100 nm, but nanoparticles can be of a larger size, for example, for particles that are oblong, tubular, and the like. Although the size of most molecules would fit into the above outline, individual molecules are usually not referred to as nanoparticles.

Nascent translation product: As used herein, the term “nascent translation product” refers to a series of linked amino acids undergoing elongation catalyzed by the ribosome. The nascent translation product is characterized by association with the ribosome. In some embodiments, association with the ribosome is in the peptide exit channel. In some embodiments, the nascent translation product is characterized by covalent association with a tRNA. In some embodiments, the nascent translation product is characterized by association with the ribosome in the peptide exit channel and covalent association with a tRNA. In some embodiments, the nascent translation product is characterized by association with the ribosome in the peptide exit channel, covalent association with a tRNA, and non-covalent association with the mRNA.

Nucleic acid: As used herein, the term “nucleic acid” is used in its broadest sense and encompasses any compound and/or substance that includes a polymer of nucleotides. These polymers are often referred to as polynucleotides. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a P-D-ribo configuration, a-LNA having an a-L-ribo configuration (a diastereomer of LNA), 2′-amino-LNA having a 2′-amino functionalization, and 2′-amino-α-LNA having a 2-amino functionalization) or hybrids thereof.

Nucleic Acid Structure: As used herein, the term “nucleic acid structure” (used interchangeably with “polynucleotide structure”) refers to the arrangement or organization of atoms, chemical constituents, elements, motifs, and/or sequence of linked nucleotides, or derivatives or analogs thereof, that comprise a nucleic acid (e.g., an mRNA). The term also refers to the two-dimensional or three-dimensional state of a nucleic acid. Accordingly, the term “RNA structure” refers to the arrangement or organization of atoms, chemical constituents, elements, motifs, and/or sequence of linked nucleotides, or derivatives or analogs thereof, comprising an RNA molecule (e.g., an mRNA) and/or refers to a two-dimensional and/or three dimensional state of an RNA molecule. Nucleic acid structure can be further demarcated into four organizational categories referred to herein as “molecular structure”, “primary structure”, “secondary structure”, and “tertiary structure” based on increasing organizational complexity.

Open Reading Frame: As used herein, the term “open reading frame”, abbreviated as “ORF”, refers to a segment or region of an mRNA molecule that encodes a polypeptide. The ORF comprises a continuous stretch of non-overlapping, in-frame codons, beginning with the initiation codon and ending with a stop codon, and is translated by the ribosome.

Operably linked: As used herein, the phrase “operably linked” refers to a functional connection between two or more molecules, constructs, transcripts, entities, moieties or the like.

Patient: As used herein, “patient” refers to a subject who may seek or be in need of treatment, requires treatment, is receiving treatment, will receive treatment, or a subject who is under care by a trained professional for a particular disease or condition. In particular embodiments, a patient is a human patient. In some embodiments, a patient is a patient suffering from cancer (e.g., liver cancer or colorectal cancer).

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio

Pharmaceutically acceptable excipient: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P.H. Stahl and C.G. Wermuth (eds.), Wiley-VCH, 2008, and Berge et al., Journal of Pharmaceutical Science, 66, 1-19 (1977), each of which is incorporated herein by reference in its entirety.

Polypeptide: As used herein, the term “polypeptide” or “polypeptide of interest” refers to a polymer of amino acid residues typically joined by peptide bonds that can be produced naturally (e.g., isolated or purified) or synthetically.

Pre-Initiation Complex: As used herein, the term “pre-initiation complex” (alternatively “43S pre-initiation complex”; abbreviated as “PIC”) refers to a ribonucleoprotein complex comprising a 40S ribosomal subunit, eukaryotic initiation factors (eIF1, eIF1A, eIF3, eIF5), and the eIF2-GTP-Met-tRNAᵢ^{Met} ternary complex, that is intrinsically capable of attachment to the 5′ cap of an mRNA molecule and, after attachment, of performing ribosome scanning of the 5′ UTR.

Polypeptide: As used herein, the term “polypeptide” or “polypeptide of interest” refers to a polymer of amino acid residues typically joined by peptide bonds that can be produced naturally (e.g., isolated or purified) or synthetically.

RNA element: As used herein, the term “RNA element” refers to a portion, fragment, or segment of an RNA molecule that provides a biological function and/or has biological activity (e.g., regulation of mRNA localization, stability or translation). Modification of a polynucleotide by the incorporation of one or more RNA elements, such as those described herein, provides one or more desirable functional properties to the modified polynucleotide. RNA elements, as described herein, can be naturally-occurring, non-naturally occurring, synthetic, engineered, or any combination thereof. For example, naturally-occurring RNA elements that provide a regulatory activity include elements found throughout the transcriptomes of viruses, prokaryotic and eukaryotic organisms (e.g., humans). RNA elements in particular eukaryotic mRNAs and translated viral RNAs have been shown to be involved in mediating many functions in cells. Exemplary natural RNA elements include, but are not limited to, translation initiation elements (e.g., internal ribosome entry site (IRES), see Kieft et al., (2001) RNA 7(2):194-206), translation enhancer elements (e.g., the APP mRNA translation enhancer element, see Rogers et al., (1999) J Biol Chem 274(10):6421-6431), mRNA stability elements (e.g., AU-rich elements (AREs), see Garneau et al., (2007) Nat Rev Mol Cell Biol 8(2):113-126), translational repression element (see e.g., Blumer et al., (2002) Mech Dev 110(1-2):97-112), protein-binding RNA elements (e.g., iron-responsive element, see Selezneva et al., (2013) J Mol Biol 425(18):3301-3310), cytoplasmic polyadenylation elements (Villalba et al., (2011) Curr Opin Genet Dev 21(4):452-457), and catalytic RNA elements (e.g., ribozymes, see Scott et al., (2009) Biochim Biophys Acta 1789(9-10):634-641). A “structural RNA element” refers to an RNA element comprising a stable RNA secondary structure that provides a biological function and/or has biological activity (e.g., regulation of mRNA localization, stability or translation).

Residence time: As used herein, the term “residence time” refers to the time of occupancy of a pre-initiation complex (PIC) or a ribosome at a discrete position or location along an mRNA molecule.

Ribosomal density: As used herein, the term “ribosomal density” refers to the quantity or number of ribosomes attached to a single mRNA molecule. Ribosomal density plays an important role in translation of mRNA into protein and affects a number of intracellular phenomena. Low ribosomal density may lead to a low translation rate, and a high degradation rate of mRNA molecules. Conversely, a ribosome density that is too high may lead to ribosomal traffic jams, collisions and abortions. It may also contribute to co-translational misfolding of proteins. In some embodiments, the RNA element(s) in an mRNA as described herein increase ribosomal density on the mRNA. In some embodiments, the RNA element(s) result in an optimal ribosomal density on the mRNA to maximize the protein translation rate.

Stability: As used herein to characterize a polynucleotide (e.g., an mRNA)), the term “stable” or “stability” refers to a reduced susceptibility to degradation or destruction (e.g., a reduced susceptibility to nuclease cleavage). For example, the term “stable” may be used to refer to a reduction in the rate of nuclease degradation (e.g., by endonuclease-mediated cleavage) of an mRNA. In certain embodiments, the half-life (t½) of an mRNA represents an objective measurement of its stability. Similarly, in certain embodiments, the amount, expression level, or enzymatic activity of an expression product that is produced following the expression (e.g., translation) of a stable or nuclease-resistant mRNA represents an objective measurement of its stability.

Stable RNA Secondary Structure: As used herein, the term “stable RNA secondary structure” refers to a structure, fold, or conformation adopted by an RNA molecule, or local segment or portion thereof, that is persistently maintained under physiological conditions and characterized by a low free energy state. Typical examples of stable RNA secondary structures include duplexes, hairpins, and stem-loops. Stable RNA secondary structures are known in the art to exhibit various biological activities.

Subject: As used herein, the term “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, a subject may be a human patient having ovarian cancer.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Targeting moiety: As used herein, a “targeting moiety” is a compound or agent that may target a nanoparticle to a particular cell, tissue, and/or organ type.

Therapeutic Agent: The term “therapeutic agent” refers to any agent that, when administered to a subject, has a therapeutic, diagnostic, and/or prophylactic effect and/or elicits a desired biological and/or pharmacological effect.

Transcription start site: As used herein, the term “transcription start site” refers to at least one nucleotide that initiates transcription by an RNA polymerase. In some embodiments, an mRNA described herein comprises a transcription start site. In some embodiments, the transcription start site initiates transcription by T7 RNA polymerase, and the transcription start site is referred to as a “T7 start site”. In some embodiments, the transcription start site comprises a single G. In some embodiments, the transcription start site comprises GG. In some embodiments, the mRNA comprises a transcription start site comprising the sequence GGGAAA.

Transcriptional Regulatory Activity: As used herein, the term “transcriptional regulatory activity” (used interchangeably with “transcriptional regulatory function”) refers to a biological function, mechanism, or process that modulates (e.g., regulates, influences, controls, varies) the activity of the transcriptional apparatus, including the activity of RNA polymerase. In some aspects, the desired transcriptional regulatory activity promotes and/or enhances the transcriptional fidelity of DNA transcription. In some aspects, the desired transcriptional regulatory activity reduces and/or inhibits leaky scanning.

Translation of a polynucleotide comprising an open reading frame encoding a polypeptide can be controlled and regulated by a variety of mechanisms that are provided by various cis-acting nucleic acid structures. For example, naturally-occurring, cis-acting RNA elements that form hairpins or other higher-order (e.g., pseudoknot) intramolecular mRNA secondary structures can provide a translational regulatory activity to a polynucleotide, wherein the RNA element influences or modulates the initiation of polynucleotide translation, particularly when the RNA element is positioned in the 5′ UTR close to the 5′-cap structure (Pelletier and Sonenberg (1985) Cell 40(3):515-526; Kozak (1986) Proc Natl Acad Sci 83:2850-2854). Cis-acting RNA elements can also affect translation elongation, being involved in numerous frameshifting events (Namy et al., (2004) Mol Cell 13(2):157-168). Internal ribosome entry sequences (IRES) represent another type of cis-acting RNA element that are typically located in 5′ UTRs, but have also been reported to be found within the coding region of naturally-occurring mRNAs (Holcik et al. (2000) Trends Genet 16(10):469-473). In cellular mRNAs, IRES often coexist with the 5′-cap structure and provide mRNAs with the functional capacity to be translated under conditions in which cap-dependent translation is compromised (Gebauer et al., (2012) Cold Spring Harb Perspect Biol 4(7):a012245). Another type of naturally-occurring cis-acting RNA element comprises upstream open reading frames (uORFs). Naturally-occurring uORFs occur singularly or multiply within the 5′ UTRs of numerous mRNAs and influence the translation of the downstream major ORF, usually negatively (with the notable exception of GCN4 mRNA in yeast and ATF4 mRNA in mammals, where uORFs serve to promote the translation of the downstream major ORF under conditions of increased eIF2 phosphorylation (Hinnebusch (2005) Annu Rev Microbiol 59:407-450)). Additional exemplary translational regulatory activities provided by components, structures, elements, motifs, and/or specific sequences comprising polynucleotides (e.g., mRNA) include, but are not limited to, mRNA stabilization or destabilization (Baker & Parker (2004) Curr Opin Cell Biol 16(3):293-299), translational activation (Villalba et al., (2011) Curr Opin Genet Dev 21(4):452-457), and translational repression (Blumer et al., (2002) Mech Dev 110(1-2):97-112). Studies have shown that naturally-occurring, cis-acting RNA elements can confer their respective functions when used to modify, by incorporation into, heterologous polynucleotides (Goldberg-Cohen et al., (2002) J Biol Chem 277(16):13635-13640).

Transfection: As used herein, the term “transfection” refers to methods to introduce a species (e.g., a polynucleotide, such as an mRNA) into a cell.

Treating: As used herein, the term “treating” refers to partially or completely alleviating, ameliorating, improving, relieving, delaying onset of, inhibiting progression of, reducing severity of, and/or reducing incidence of one or more symptoms or features of ovarian cancer. For example, “treating” cancer may refer to inhibiting survival, growth, and/or spread of a tumor. Treatment may be measured by reduction in numbers of tumors or reduction in size of a particular tumor and/or reduction in metastasis. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Preventing: As used herein, the term “preventing” refers to partially or completely inhibiting the onset of one or more symptoms or features of a particular infection, disease, disorder, and/or condition.

Tumor: As used herein, a “tumor” is an abnormal growth of tissue, whether benign or malignant.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

### Equivalents and Scope

Those skilled in the art will recognize, or be able to ascertain using no more than routine experimentation, many equivalents to the specific embodiments in accordance with the disclosure described herein. The scope of the present disclosure is not intended to be limited to the Description below, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

### OTHER EMBODIMENTS

E1. A messenger RNA (mRNA) comprising:
a 5′ cap;
a 5′ untranslated region (UTR) comprising a structural RNA element comprising a stem-loop;
an initiation codon;
a full open reading frame encoding a polypeptide; and
a 3′ UTR,
wherein the structural RNA element comprises a sequence of linked nucleotides, wherein each nucleotide comprises a nucleobase selected from the group consisting of: adenine, guanine, thymine, uracil, and cytosine, or derivatives or analogs thereof, and wherein the structural RNA element provides a translational regulatory activity selected from:
   a. increasing residence time of a 43S pre-initiation complex (PIC) or ribosome at, or proximal to, the initiation codon;
   b. increasing initiation of polypeptide synthesis at or from the initiation codon;
   c. increasing an amount of polypeptide translated from the full open reading frame;
   d. increasing fidelity of initiation codon decoding by the PIC or ribosome;
   e. inhibiting or reducing leaky scanning by the PIC or ribosome;
   f. decreasing a rate of decoding the initiation codon by the PIC or ribosome;
   g. inhibiting or reducing initiation of polypeptide synthesis at any codon within the mRNA other than the initiation codon;
   h. inhibiting or reducing the amount of polypeptide translated from any open reading frame within the mRNA other than the full open reading frame;
   i. inhibiting or reducing the production of aberrant translation products;
   j. increasing ribosomal density on the mRNA; and
   k. a combination of any of (a)-(j).
      E2. The mRNA of embodiment E1, wherein the structural RNA element comprises a nucleotide sequence of about 10-30 nucleotides, about 15-25 nucleotides, or about 20-25 nucleotides.
      E3. The mRNA of embodiment E2, wherein the structural RNA element comprises a nucleotide sequence of about 15-25 nucleotides.
      E4. The mRNA of embodiment E1, wherein the structural RNA element comprises a nucleotide sequence of about 30, 29, 28, 27, 26, 25, 24, 23, 22, 21, 20, 19, 18, 17, 16, 15, 14, 13, 12, or about 10 nucleotides in length.
      E5. The mRNA of any one of embodiments E1-E4, wherein the structural RNA element comprises a double-stranded stem comprising about 3-8 base pairs, about 4-7 base pairs, about 5-6 base pairs, or about 3, 4, 5, 6, 7, or 8 base pairs.
      E6. The mRNA of embodiment E5, wherein the double-stranded stem comprises about 4-7 base pairs.
      E7. The mRNA of any one of embodiments E5 or E6, wherein the double-stranded stem comprises at least 50% G/C base pairs.
      E8. The mRNA of any one of embodiments E5-E7, wherein the double-stranded stem comprises at least 60%, at least 65%, at least 70%, at least 75%, at least 80%, at least 85%, or at least 90% G/C base pairs.
      E9. The mRNA of any one of embodiments E5-E7, wherein the double-stranded stem comprises 30% or less A/U base pairs.
      E10. The mRNA of any one of embodiments E1-E9, wherein the structural RNA element stem-loop comprises a single-stranded loop of about 3-8 nucleotides, about 4-7 nucleotides, about 5-6 nucleotides, about 3, 4, 5, 6, 7, or 8 nucleotides in length.
      E11. The mRNA of embodiment E10, wherein the single-stranded loop is about 4-7 nucleotides in length.
      E12. The mRNA of any one of embodiments E1-E11, wherein the structural RNA element comprises at least 50%, at least 60%, at least 65%, at least 70%, at least 75%, at least 80%, at least 85%, or at least 90% G/C bases.
      E13. The mRNA of embodiment 12, wherein the structural RNA element comprises at least 60% G/C bases.
      E14. The mRNA of any one of embodiments E12 or E13, wherein the structural RNA element comprises 40% or less A/U bases.
      E15. The mRNA of embodiment E1, wherein the structural RNA element comprises a nucleotide sequence at least 80%, 85%, 90%, 95%, 96%, 97%, 98%, or 99% identical to the nucleotide sequence of SEQ ID NO: 6.
      E16. The mRNA of embodiment E1, wherein the structural RNA element comprises a nucleotide sequence which differs from SEQ ID NO: 6 by substitution, deletion, or insertion of 1, 2, 3, 4, or 5 nucleotides.
      E17. The mRNA of embodiment E1, wherein the structural RNA element comprises a double-stranded stem of about 4-7 base pairs and a nucleotide sequence which differs from SEQ ID NO: 6 by substitution, deletion or insertion of 1, 2, 3, 4, or 5 nucleotides. E18. The mRNA of embodiment E1, wherein the structural RNA element comprises a single-stranded loop of about 4-7 bases and a nucleotide sequence which differs from SEQ ID NO: 6 by substitution, deletion or insertion of 1, 2, 3, 4, or 5 nucleotides.
      E19. The mRNA of any one of embodiments E15-E18, wherein the structural RNA element comprises at least 50%, at least 60%, at least 65%, at least 70%, at least 75%, at least 80%, at least 85%, or at least 90% G/C bases.
      E20. The mRNA of embodiment E1, wherein the structural RNA element comprises a nucleotide sequence at least 80%, 85%, 90%, 95%, 96%, 97%, 98%, or 99% identical to the nucleotide sequence of SEQ ID NO: 47.
      E21. The mRNA of embodiment E1, wherein the structural RNA element comprises a nucleotide sequence which differs from SEQ ID NO: 47 by substitution, deletion or insertion of 1, 2, 3, 4, or 5 nucleotides.
      E22. The mRNA of embodiment E1, wherein the structural RNA element comprises a double-stranded stem of about 4-7 base pairs and a nucleotide sequence which differs from SEQ ID NO: 47 by substitution, deletion or insertion of 1, 2, 3, 4, or 5 nucleotides.
      E23. The mRNA of embodiment E1, wherein the structural RNA element comprises a single-stranded loop of about 4-7 bases and a nucleotide sequence which differs from SEQ ID NO: 47 by substitution, deletion or insertion of 1, 2, 3, 4, or 5 nucleotides.
      E24. The mRNA of any one of embodiments E21-E23, wherein the structural RNA element comprises at least 50%, at least 60%, at least 65%, at least 70%, at least 75%, at least 80%, at least 85%, or at least 90% G/C bases.
      E25. The mRNA of any one of embodiments E1-E24, wherein the structural RNA element has a deltaG (ΔG) of about −20 to −30 kcal/mol, about −20 to −25 kcal/mol, about −15 to −20 kcal/mol, about −10 to −15 kcal/mol, or about −5 to −10 kcal/mol.
      E26. An mRNA comprising:
a 5′ cap;
a 5′ UTR comprising a structural RNA element comprising a stem-loop;
an ORF encoding a polypeptide; and
a 3′ UTR,
wherein the structural RNA element comprises a sequence of 15-25 linked nucleotides, wherein each nucleotide comprises a nucleobase selected from the group consisting of: adenine, guanine, thymine, uracil, and cytosine, or derivatives or analogs thereof, and wherein the structural RNA element comprises (i) a double-stranded stem of about 4-7 base pairs comprising at least 50% G/C base pairs; (ii) a single-stranded loop of about 3-8 nucleotides; and (iii) a deltaG (ΔG) about −10 to −15 kcal/mol.
   E27. An mRNA comprising:
a 5′ cap;
a 5′ UTR comprising a structural RNA element comprising a stem-loop;
an ORF encoding a polypeptide; and
a 3′ UTR,
wherein the structural RNA element comprises a sequence of 15-25 linked nucleotides, wherein each nucleotide comprises a nucleobase selected from the group consisting of: adenine, guanine, thymine, uracil, and cytosine, or derivatives or analogs thereof, and wherein the structural RNA element comprises (i) a double-stranded stem of about 4-7 base pairs; (ii) a single-stranded loop of about 3-8 nucleotides; (iii) a nucleotide sequence which differs from SEQ ID NO: 6 or SEQ ID NO: 47 by substitution, deletion or insertion of 1, 2, 3, 4, or 5 nucleotides; and (iv) a deltaG (ΔG) about −10 to −15 kcal/mol.
   E28. The mRNA of any one of embodiments E26 or E27, wherein the double-stranded stem comprises at least 60%, at least 65%, at least 70%, at least 75%, at least 80%, at least 85%, or at least 90% G/C base pairs.
   E29. The mRNA of any one of embodiments E26-E28, wherein the double-stranded stem comprises 30% or less A/U base pairs.
   E30. The mRNA of any one of embodiments E26-E29, wherein the single-stranded loop is about 4-7 nucleotides in length.
   E31. The mRNA of any one of embodiments E26-E30, wherein the structural RNA element comprises at least 50%, at least 60%, at least 65%, at least 70%, at least 75%, at least 80%, at least 85%, or at least 90% G/C bases.
   E32. The mRNA of embodiment E31, wherein the structural RNA element comprises at least 60% G/C bases.
   E33. The mRNA of any one of embodiments E31 or E32, wherein the structural RNA element comprises 40% or less A/U bases.
   E34. An mRNA comprising:
a 5′ cap;
a 5′ UTR comprising a structural RNA element comprising a stem-loop;
an ORF encoding a polypeptide; and
a 3′ UTR,
wherein the structural RNA element comprises (i) a nucleotide sequence at least 80%, 85%, 90%, 95%, 96%, 97%, 98%, or 99% identical to the nucleotide sequence of SEQ ID NO: 6 or the nucleotide sequence of SEQ ID NO: 47.
   E35. The mRNA of embodiment E34, wherein the structural RNA element comprises a nucleotide sequence at least 80%, 85%, 90%, 95%, 96%, 97%, 98%, or 99% identical to the nucleotide sequence of SEQ ID NO: 6.
   E36. The mRNA of embodiment E34, wherein the structural RNA element comprises a nucleotide sequence at least 80%, 85%, 90%, 95%, 96%, 97%, 98%, or 99% identical to the nucleotide sequence of SEQ ID NO: 47.
   E37. The mRNA of any one of embodiments E34-E36, wherein the structural RNA element has a deltaG (ΔG) of about −20 to −25 kcal/mol, about −15 to −20 kcal/mol, or about −10 to −15 kcal/mol.
   E38. The mRNA of embodiment E37, wherein the structural RNA element has a deltaG (ΔG) about −10 to −15 kcal/mol.
   E39. The mRNA of any one of the preceding embodiments, wherein the structural RNA element provides a translational regulatory activity comprising increasing an amount of polypeptide translated from the full open reading frame.
   E40. An mRNA comprising:
a 5′ cap;
a 5′ UTR comprising a structural RNA element comprising the nucleotide sequence of SEQ ID NO: 6;
an ORF encoding a polypeptide; and a 3′ UTR.
   E41. An mRNA comprising:
a 5′ cap;
a 5′ UTR comprising a structural RNA element comprising the nucleotide sequence of SEQ ID NO: 47;
an ORF encoding a polypeptide; and a 3′ UTR.
   E42. The mRNA of any one of embodiments E1-E41, wherein the 5′ UTR comprises a Kozak-like sequence upstream of the initiation codon and the structural RNA element is located upstream of the Kozak-like sequence in the 5′ UTR.
   E43. The mRNA of embodiment E42, wherein the structural RNA element is located about 45-50, about 40-45, about 35-40, about 30-35, about 25-30, about 20-25, about 15-20, about 10-15, about 6-10 nucleotides, about 1-5 nucleotides, or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide upstream of the Kozak-like sequence in the 5′ UTR.
   E44. The mRNA of embodiment E43, wherein the structural RNA element is located about 40-45 nucleotides upstream of the Kozak-like sequence in the 5′ UTR.
   E45. The mRNA of embodiment E43, wherein the structural RNA element is located about 10-15 nucleotides upstream of the Kozak-like sequence in the 5′ UTR.
   E46. The mRNA of embodiment E43, wherein the structural RNA element is located about 6-10 nucleotides upstream of the Kozak-like sequence in the 5′ UTR.
   E47. The mRNA of any one of embodiments E1-E43, wherein the structural RNA element is located downstream of the 5′ cap or 5′ end of the mRNA in the 5′ UTR.
   E48. The mRNA of embodiment E47, wherein the structural RNA element is located about 45-50, about 40-45, about 35-40, about 30-35, about 25-30, about 20-25, about 15-20, about 10-15, about 5-10 nucleotides, about 1-5 nucleotide(s), or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) downstream of the 5′ cap or 5′ end of the mRNA in the 5′ UTR.
   E49. The mRNA of embodiment E48, wherein the structural RNA element is located about 40-45 nucleotides downstream of the 5′ cap or 5′ end of the mRNA in the 5′ UTR.
   E50. The mRNA of embodiment E48, wherein the structural RNA element is located about 20-25 nucleotides downstream of the 5′ cap or 5′ end of the mRNA in the 5′ UTR.
   E51. The mRNA of embodiment E36, wherein the structural RNA element is located about 5-10 nucleotides downstream of the 5′ cap or 5′ end of the mRNA in the 5′ UTR.
   E52. The mRNA of any one of the preceding embodiments, comprising a Kozak-like sequence in the 5′UTR, wherein the 5′UTR comprises a GC-rich RNA element comprising a sequence of about 20-30, about 10-20, about 10-15, about 5-15, or about 3-15 nucleotides, or derivatives or analogs thereof, wherein the sequence is at least about 50% cytosine, and wherein the GC-rich RNA element is located upstream of the Kozak-like in the 5′ UTR. E53. The mRNA of embodiment E52, wherein the GC-rich RNA element comprises a sequence of about 3-15, about 15, 14, 13, 12, 11, 10, 9, 8, 7, 6, 5, 4, or 3 nucleotides, or derivatives or analogs thereof, wherein the sequence is about 50%-60% cytosine, about 60%-70% cytosine, or about 70%-80% cytosine.
   E54. The mRNA of any one of embodiments E52 or E53, wherein the GC-rich RNA element comprises a sequence of cytosine and guanine.
   E55. The mRNA of embodiment E54, wherein the GC-rich RNA element comprises a sequence of about 3-30 guanine and cytosine nucleotides, or derivatives or analogues thereof, wherein the sequence comprises a repeating GC-motif, wherein the repeating GC-motif is [CCG]ₙ or [GCC]ₙ, wherein n=1 to 10, 1-5, 3, 2 or 1.
   E56. The mRNA of embodiment E54, wherein the sequence of the GC-rich RNA element is selected from (i) the sequence of EK1 [CCCGCC] set forth in SEQ ID NO: 3; (ii) the sequence of EK2 [GCCGCC] set forth in SEQ ID NO: 18; and (iii) the sequence of EK3 [CCGCCG] set forth in SEQ ID NO: 19.
   E57. The mRNA of embodiment E54, wherein the sequence of the GC-rich RNA element comprises the sequence of V1 [CCCCGGCGCC] set forth in SEQ ID NO: 1.
   E58. The mRNA of embodiment E54, wherein the sequence of the GC-rich RNA element comprises the sequence of V2 [CCCCGGC] set forth in SEQ ID NO: 2.
   E59. The mRNA of embodiment E54, wherein the sequence of the GC-rich RNA element comprises the sequence of CG1 [GCGCCCCGCGGCGCCCCGCG] set forth in SEQ ID NO: 20.
   E60. The mRNA of embodiment E54, wherein the sequence of the GC-rich RNA element comprises the sequence of CG2 [CCCGCCCGCCCCGCCCCGCC] set forth in SEQ ID NO: 21.
   E61. The mRNA of any one of embodiments E52-E60, wherein the GC-rich RNA element is located about 20-30, about 15-20, about 10-15, about 5-10, or about 1-5 nucleotides upstream of the Kozak-like sequence in the 5′ UTR.
   E62. The mRNA of embodiment E61, wherein the GC-rich RNA element is located about 5, about 4, about 3, about 2, or 1 nucleotide(s) upstream of the Kozak-like sequence in the 5′ UTR.
   E63. The mRNA of any one of embodiments E52-E60, wherein the GC-rich RNA element is upstream of and immediately adjacent to the Kozak-like sequence in the 5′ UTR.
   E64. The mRNA of any one of embodiments E52-E63, wherein the Kozak-like sequence comprises the sequence [5‘-GCCACC-’3] set forth in SEQ ID NO: 17 or [5′-GCCGCC-′3] set forth in SEQ ID NO: 48.
   E65. The mRNA of any one of embodiments E52-E64, wherein the GC-rich RNA element comprises a stable RNA secondary structure located downstream of the initiation codon.
   E66. The mRNA of embodiment E65, wherein the stable RNA secondary structure is a hairpin or a stem-loop.
   E67. The mRNA of embodiment E66, wherein the stable RNA secondary structure has a deltaG of about −20 to −30 kcal/mol, about −10 to −20 kcal/mol, or about −5 to −10 kcal/mol.
   E68. The mRNA of embodiment E65, wherein the GC-rich RNA element comprising a stable RNA secondary structure selected from (i) the sequence of SL1 [CCGCGGCGCCCCGCGG] as set forth in SEQ ID NO: 24; (ii) the sequence of SL2 [GCGCGCAUAUAGCGCGC] as set forth in SEQ ID NO: 25; (iii) the sequence of SL3 [CAUGGUGGCGGCCCGCCGCCACCAUG] as set forth in SEQ ID NO: 49; (iv) the sequence of SL4 [CAUGGUGGCCCGCCGCCACCAUG] as set forth in SEQ ID NO: 50; and (v) the sequence of SL5 [CAUGGUGCCCGCCGCCACCAUG] as set forth in SEQ ID NO: 51.
   E69. The mRNA of any one of embodiments E65-E68, wherein the GC-rich RNA element is located about 20-30, about 10-20, about 15-20, about 10-15, about 5-10, or about 1-5 nucleotides downstream of the initiation codon.
   E70. The mRNA of any one of the preceding embodiments, wherein the 5′ UTR comprises a C-rich RNA element located proximal to the 5′ cap, wherein the C-rich RNA element comprises a sequence of about 3-20 nucleotides, wherein the sequence comprises about 50-55%, 55-60%, 60-65%, 70-75%, 75-80%, 80-85%, 85-90% or 90-95%, or about 95%, about 90%, about 85%, about 80%, about 75%, about 70%, about 65%, about 60%, about 55%, or about 50% cytosine nucleobases or derivatives or analogs thereof.
   E71. The mRNA of embodiment E70, wherein the C-rich RNA element comprises a sequence of about 3-20 nucleotides, about 4-18 nucleotides, about 6-16 nucleotides, about 6-14 nucleotides, about 6-12 nucleotides, about 6-10 nucleotides, or about 20, 19, 18, 17, 16, 15, 14, 13, 12, 11, 10, 9, 8, 7, 6, 5, 4, or 3 nucleotides.
   E72. The mRNA of any one of embodiments E70 or E71, wherein the C-rich RNA element comprises a sequence of about 6-12 nucleotides, wherein the sequence comprises 70-75%, 75-80%, 80-85%, 85-90% or 90-95% cytosine nucleobases, or derivatives or analogs thereof, optionally wherein the sequence is less than about 30-25%, 25-20%, 20-15%, 15-10%, or 10-5% adenosine and/or guanosine nucleobases, or derivatives or analogs thereof.
   E73. The mRNA of any one of embodiments E70-E71, wherein the C-rich RNA element comprises a sequence of linked nucleotides comprising the formula:

5′-[C1]ᵥ-[N1]_{w}-[N2]ₓ-[N3]_{y},-[C2]_{z}-3′,
wherein C1 and C2 are nucleotides comprising cytidine, or a derivative or analogue thereof, wherein N1, and N2 and N3 if present, are each a nucleotide comprising a nucleobase selected from the group consisting of: adenine, guanine, thymine, uracil, and cytosine, and derivatives or analogues thereof, wherein v, w, x, y and z are integers whose value indicates the number of nucleotides comprising the C-rich RNA element, wherein v=2-15 nucleotides, wherein w=1-5 nucleotides, wherein x=0-5 nucleotides, wherein y=0-5 nucleotides, and wherein z=2-10 nucleotides.
   E74. The mRNA of embodiment E73, wherein v=3-12 nucleotides, 5-10 nucleotides, 6-8 nucleotides, 3, 4, 5, 6, 7, 8, 9 or 10 nucleotides.
   E75. The mRNA of any one of embodiments E73-E74, wherein z=2-7 nucleotides, 3-5 nucleotides, 2, 3, 4, 5, 6, or 7 nucleotides.
   E76. The mRNA of any one of embodiments E73-E75, wherein w=1-3 nucleotides, 1, 2, or 3 nucleotide(s).
   E77. The mRNA of any one of embodiments E73-E75, wherein x=0-3 nucleotides, 0, 1, 2, or 3 nucleotide(s).
   E78. The mRNA of any one of embodiments E73-E75, wherein y=0-3 nucleotides, 0, 1, 2, or 3 nucleotide(s).
   E79. The mRNA of any one of embodiments E73-E75, wherein N1 comprises adenosine, or derivative or analogue thereof; w=1 or 2; x=0, 1, 2, or 3; and y=0, 1, 2, or 3.
   E80. The mRNA of any one of embodiments E73-E75, wherein N1 comprises adenosine, or derivative or analogue thereof; w=1 or 2; x=0; and y=0.
   E81. The mRNA of any one of embodiments E73-E75, wherein N1 comprises uracil, or derivative or analogue thereof; w=1 or 2; N2 comprises adenosine, or derivative or analogue thereof; x=1, 2, or 3; N3 is guanosine, or derivative or analogue thereof; and y=1 or 2.
   E82. The mRNA of any one of embodiments E73-E75, wherein N1 comprises uracil, or derivative or analogue thereof; w=1; N2 comprises adenosine, or derivative or analogue thereof; x=2; N3 is guanosine, or derivative or analogue thereof; and y=1.
   E83. The mRNA of embodiments E70-E71, wherein the C-rich RNA element comprises the formula

5′-[C1]ᵥ-[N1]_{w}-[N2]ₓ-[N3]_{y},-[C2]_{z}-3′,

wherein C₁ and C₂ are nucleotides comprising cytidine, or a derivative or analogue thereof, wherein N1, and N2 and N3 if present, are each a nucleotide comprising a nucleobase selected from the group consisting of: adenine, guanine, and uracil, and derivatives or analogues thereof, wherein v, w, x, y and z are integers whose value indicates the number of nucleotides comprising the C-rich RNA element, wherein v=4-10 nucleotides, wherein w=1-3 nucleotides, wherein x=0-3 nucleotides, wherein y=0-3 nucleotides, and wherein z=2-6 nucleotides.
E84. The mRNA of embodiment E83, wherein v=6-8 nucleotides, 6, 7, or 8 nucleotides.
E85. The mRNA of any one of embodiments E83-E84, wherein z=2-5 nucleotides, 2, 3, 4, or 5 nucleotides.
E86. The mRNA of any one of embodiments E83-E84, wherein w=1 or 2 nucleotide(s).
E87. The mRNA of any one of embodiments E83-E84, wherein x=0, 1 or 2 nucleotide(s).
E88. The mRNA of any one of embodiments 83-84, wherein y=0 or 1 nucleotide(s).
E89. The mRNA of any one of embodiments E83-E85, wherein N1 comprises adenosine, or derivative or analogue thereof; w=1; x=0; and y=0.
E90. The mRNA of any one of embodiments E83-E85, wherein N1 comprises adenosine, or derivative or analogue thereof; w=2; x=0; and y=0.
E91. The mRNA of any one of embodiments E83-E85, wherein N1 comprises uracil, or derivative or analogue thereof; w=1 or 2; N2 comprises adenosine, or derivative or analogue thereof; x=1, 2, or 3; N3 is guanosine, or derivative or analogue thereof; and y=1 or 2.
E92. The mRNA of any one of embodiments E83-E85, wherein N1 comprises uracil, or derivative or analogue thereof; w=1; N2 comprises adenosine, or derivative or analogue thereof; x=2; N3 is guanosine, or derivative or analogue thereof; and y=1.
E93. The mRNA of any one of embodiments E83-E85, wherein v=6-8; N1 comprises adenosine, or derivative or analogue thereof; w=1 or 2; x=0; y=0; and z=2-5.
E94. The mRNA of any one of embodiments E83-E85, wherein v=6-8; N1 comprises uracil, or derivative or analogue thereof; w=1; N2 comprises adenosine, or derivative or analogue thereof; x=2; N3 is guanosine, or derivative or analogue thereof; y=1; and z=2-5.
E95. The mRNA of embodiment E83, wherein the C-rich RNA element comprises the nucleotide sequence [5′-CCCCCCCCAACC-3′] set forth in SEQ ID NO 30 or comprises the nucleotide sequence [5′-CCCCCCCAACCC-3′] set forth in SEQ ID NO: 29.
E96. The mRNA of embodiment E83, wherein the C-rich RNA element comprises the nucleotide sequence [5′-CCCCCCACCCCC-3′] set forth in SEQ ID NO: 31.
E97. The mRNA of embodiment E83, wherein the C-rich RNA element comprises the nucleotide sequence [5′-CCCCCCUAAGCC-3′] set forth in SEQ ID NO: 32.
E98. The mRNA of embodiment E83, wherein the C-rich RNA element comprises the nucleotide sequence [5′-CCCCACAACC-3′] set forth in SEQ ID NO: 33, or the nucleotide sequence [5′-CCCCCACAACC-3′] set forth in SEQ ID NO: 34.
E99. The mRNA of any one of embodiments E70-E98, wherein the C-rich RNA element is located about 40-50, about 30-40, about 20-30, about 10-20 or about 5-10 nucleotides downstream of the 5′ cap or 5′ end of the mRNA in the 5′ UTR.
E100. The mRNA of any one of embodiments E70-E98, wherein the C-rich RNA element is located about 15-20, about 10-15, about 5-10 nucleotides, about 1-5 nucleotides, or about 20, 19, 18, 17, 16, 15, 14, 13, 12, 11, 10, 9, 8, 7, 6, 5, 4, 3, 2 or 1 nucleotide downstream of the 5′ cap or 5′ end of the mRNA in the 5′ UTR.
E101. The mRNA of any one of embodiments E70-E98, wherein the C-rich RNA element is located about 5-10 nucleotides downstream of the 5′ cap or 5′end of the mRNA in the 5′ UTR.
E102. An mRNA comprising a 5′ cap, a 5′ UTR, an ORF encoding a polypeptide, and a 3′ UTR,
wherein the 5′UTR comprises:
(i) a structural RNA element comprising a stem loop comprising a nucleotide sequence at least 80%, 85%, 90%, 95%, 96%, 97%, 98%, 99% or 100% identical to the nucleotide sequence of SEQ ID NO: 6 or the nucleotide sequence of SEQ ID NO: 47; and
(ii) a GC-rich RNA element comprising a nucleotide sequence selected from the group consisting of: SEQ ID NO: 1, SEQ ID NO: 2, SEQ ID NO: 3, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 49, SEQ ID NO: 50 and SEQ ID NO: 51.
   E103. The mRNA of embodiment E102, wherein the structural RNA element comprises a nucleotide sequence at least 80%, 85%, 90%, 95%, 96%, 97%, 98%, 99% or 100% identical to the nucleotide sequence of SEQ ID NO: 6.
   E104. The mRNA of embodiment E102, wherein the structural RNA element comprises a nucleotide sequence at least 80%, 85%, 90%, 95%, 96%, 97%, 98%, 99% or 100% identical to the nucleotide sequence of SEQ ID NO: 47.
   E105. The mRNA of any one of embodiments E102-E104, wherein the structural RNA element has a deltaG (ΔG) of about −20 to −25 kcal/mol, about −15 to −20 kcal/mol, or about −10 to −15 kcal/mol.
   E106. The mRNA of embodiment E102, wherein the structural RNA element comprises the nucleotide sequence of SEQ ID NO: 6.
   E107. The mRNA of embodiment E102, wherein the structural RNA element comprises the nucleotide sequence of SEQ ID NO: 47.
   E108. The mRNA of any one of embodiments E102-E107, wherein the GC-rich RNA element comprises a nucleotide sequence selected from the group consisting of: SEQ ID NO: 1, SEQ ID NO: 2 and SEQ ID NO: 23.
   E109. The mRNA of any one of embodiments E102-E107, wherein the GC-rich RNA element comprises the nucleotide sequence of SEQ ID NO: 1.
   E110. The mRNA of any one of embodiments E102-E109, wherein the mRNA comprises a Kozak-like sequence, and wherein the GC-rich RNA element is located about 1-20 nucleotides upstream of the Kozak-like sequence in the 5′ UTR.
   E111. The mRNA of embodiment E110, wherein the GC-rich RNA element is located about 5, about 4, about 3, about 2, or 1 nucleotide upstream of the Kozak-like sequence in the 5′ UTR.
   E112. The mRNA of any one of embodiments E102-E109, wherein the GC-rich RNA element is upstream of and immediately adjacent to the Kozak-like sequence in the 5′ UTR.
   E113. The mRNA of any one of embodiments E102-E112, wherein the structural RNA element that is upstream of the GC-rich RNA element in the 5′UTR.
   E114. The mRNA of embodiment E113, wherein the structural RNA element is about 1-5, 5-10, 10-20, 20-30, 30-40, or 40-50 nucleotides, or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) upstream of the GC-rich RNA element in the 5′UTR.
   E115. The mRNA of embodiment E114, wherein the structural RNA element is 1-5 nucleotides upstream of the GC-rich RNA element in the 5′UTR.
   E116. The mRNA of embodiment E114, wherein the structural RNA element is 10-20 nucleotides upstream of the GC-rich RNA element in the 5′UTR.
   E117. The mRNA of embodiment E114, wherein the structural RNA element is 30-40 nucleotides upstream of the GC-rich RNA element in the 5′UTR.
   E118. The mRNA of embodiment E113, wherein the structural RNA element is upstream of and immediately adjacent to the GC-rich RNA element in the 5′UTR.
   E119. The mRNA of any one of embodiments E102-E118, wherein the Kozak-like sequence comprises the nucleotide sequence [5′-GCCACC-3′] set forth in SEQ ID NO: 17 or the nucleotide sequence [5′-GCCGCC-3′] set forth in SEQ ID NO: 48.
   E120. The mRNA of any one of embodiments E102-E119, wherein the structural RNA element is located downstream of the 5′ cap or 5′ end of the mRNA in the 5′ UTR.
   E121. The mRNA of embodiment E120, wherein the structural RNA element is located about 45-50, about 40-45, about 35-40, about 30-35, about 25-30, about 20-25, about 15-20, about 10-15, about 5-10 nucleotides, about 1-5 nucleotide(s), or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) downstream of the 5′ cap or 5′ end of the mRNA in the 5′ UTR.
   E122. The mRNA of embodiment E121, wherein the structural RNA element is located about 40-45 nucleotides downstream of the 5′ cap or 5′ end of the mRNA in the 5′ UTR.
   E123. The mRNA of embodiment E121, wherein the structural RNA element is located about 20-25 nucleotides downstream of the 5′ cap or 5′ end of the mRNA in the 5′ UTR.
   E124. The mRNA of embodiment E121, wherein the structural RNA element is located about 5-10 nucleotides downstream of the 5′ cap or 5′ end of the mRNA in the 5′ UTR.
   E125. The mRNA of any one of embodiments E102-E119, wherein the structural RNA element is located downstream of the 5′ cap or 5′ end of the mRNA and immediately adjacent to a transcription start site element in the 5′ UTR.
   E126. The mRNA of embodiment E125, wherein the transcription start site element comprises the nucleotide sequence [5′-GGGAAA-3′] set forth in SEQ ID NO: 53 or the nucleotide sequence [5′-AGGAAA-3′] set forth in SEQ ID NO: 54.
   E127. The mRNA of any one of embodiments E102-E126, wherein the 5′UTR comprises a C-rich RNA element comprising a nucleotide sequence selected from the group consisting of: SEQ ID NO: 29, SEQ ID NO: 30, SEQ ID NO: 31, SEQ ID NO: 32, SEQ ID NO: 33 and SEQ ID NO: 34.
   E128. The mRNA embodiment E127, wherein the C-rich RNA element is proximal to the 5′ cap or 5′ end of the mRNA and upstream of each of the structural RNA element and the GC-rich RNA element in the 5′UTR.
   E129. The mRNA of embodiment E128, wherein the C-rich RNA element is about 1-5, 5-10, 10-20, 20-30, 30-40, or 40-50 nucleotides, or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) upstream of the structural RNA element in the 5′UTR.
   E130. The mRNA of embodiment E129, wherein the C-rich RNA element is 20-30 nucleotides upstream of the structural RNA element in the 5′UTR.
   E131. The mRNA of embodiment E130, wherein the C-rich RNA element is 30-40 nucleotides upstream of the structural RNA element in the 5′UTR.
   E132. The mRNA of embodiment E129, wherein the C-rich RNA element is 40-50 nucleotides upstream of the structural RNA element in the 5′UTR.
   E133. The mRNA of embodiment E127, wherein the C-rich RNA element is located downstream of the 5′ cap or 5′ end of the mRNA and upstream of each of the structural RNA element and the GC-rich RNA element in the 5′UTR.
   E134. The mRNA of embodiment E133, wherein the C-rich RNA element is located about 20-25, about 15-20, about 10-15, about 5-10 nucleotides, about 1-10, about 1-8, about 1-6, or about 1-3 nucleotide(s), or about 20, 19, 18, 17, 16, 15, 14, 13, 12, 11, 10, 9, 8, 7, 6, 5, 4, 3, 2 or 1 nucleotide(s) downstream of the 5′ cap or 5′ end of the mRNA in the 5′ UTR.
   E135. The mRNA of embodiment E134, wherein the C-rich RNA element is located about 1-10 nucleotides downstream of the 5′ cap or 5′ end of the mRNA in the 5′ UTR. E136. The mRNA of embodiment E134, wherein the C-rich RNA element is located about 5-10 nucleotides downstream of the 5′ cap or 5′ end of the mRNA in the 5′ UTR.
   E137. The mRNA of embodiment E134, wherein the C-rich RNA element is located about 1-6 nucleotides downstream of the 5′ cap or 5′ end of the mRNA in the 5′ UTR.
   E138. The mRNA of embodiment E127, wherein the C-rich RNA element is downstream of immediately adjacent to a transcription start site element and upstream of each of the structural RNA element and the GC-rich RNA element in the 5′UTR.
   E139. The mRNA of embodiment E138, wherein the transcription start site element comprises the nucleotide sequence [5′-GGGAAA-3′] set forth in SEQ ID NO: 53 or the nucleotide sequence [5′-AGGAAA-3′] set forth in SEQ ID NO: 54.
   E140. An mRNA comprising a 5′ cap, a 5′ UTR comprising a Kozak-like sequence upstream of an initiation codon, an ORF encoding a polypeptide, and a 3′ UTR,
wherein the 5′ UTR comprises from 5′ to 3′:
(i) a C-rich RNA element located proximal to the 5′ cap, wherein the C-rich RNA element comprises a nucleotide sequence selected from selected from the group consisting of SEQ ID NO: 31, SEQ ID NO: 32 and SEQ ID NO: 33;
(ii) a structural RNA element comprising a stem loop located downstream of the C-rich RNA element, wherein the structural RNA element comprises the nucleotide sequence of SEQ ID NO: 6 or the nucleotide sequence of SEQ ID NO: 47; and
(iii) a GC-rich RNA element located downstream of the structural RNA element and proximal to the Kozak-like sequence, wherein the GC-rich RNA element comprises a nucleotide sequence selected from the group consisting of: SEQ ID NO: 1, SEQ ID NO: 2 and SEQ ID NO: 23.
   E141. The mRNA of embodiment E140, wherein (i) the C-rich RNA element comprises the nucleotide sequence set forth in SEQ ID NO: 31; (ii) the structural RNA element comprises the nucleotide sequence of SEQ ID NO: 6; and (iii) the GC-rich RNA element comprises the nucleotide sequence set forth in SEQ ID NO: 1.
   E142. The mRNA of embodiment E140, wherein (i) the C-rich RNA element comprises the nucleotide sequence set forth in SEQ ID NO: 33; (ii) the structural RNA element comprises the nucleotide sequence of SEQ ID NO: 6; and (iii) the GC-rich RNA element comprises the nucleotide sequence set forth in SEQ ID NO: 1.
   E143. The mRNA of embodiment E140, wherein (i) the C-rich RNA element comprises the nucleotide sequence set forth in SEQ ID NO: 32; (ii) the structural RNA element comprises the nucleotide sequence of SEQ ID NO: 6; and (iii) the GC-rich RNA element comprises the nucleotide sequence set forth in SEQ ID NO: 23.
   E144. The mRNA of any one of embodiments E140-E143, wherein the C-rich RNA element is located about 10-15, about 5-10 nucleotides, about 1-10, about 1-8, about 1-6, or about 1-3 nucleotide(s), or about 15, 14, 13, 12, 11, 10, 9, 8, 7, 6, 5, 4, 3, 2 or 1 nucleotide(s) downstream of the 5′ cap or 5′ end of the mRNA in the 5′ UTR.
   E145. The mRNA of embodiment E144, wherein the C-rich RNA element is located about 1-10 nucleotides downstream of the 5′ cap or 5′ end of the mRNA in the 5′ UTR.
   E146. The mRNA of embodiment E144, wherein the C-rich RNA element is located about 5-10 nucleotides downstream of the 5′ cap or 5′ end of the mRNA in the 5′ UTR.
   E147. The mRNA of embodiment E144, wherein the C-rich RNA element is located about 1-6 nucleotides downstream of the 5′ cap or 5′ end of the mRNA in the 5′ UTR.
   E148. The mRNA of any one of embodiments E140-E143, wherein the C-rich RNA element is downstream of immediately adjacent to a transcription start site element, wherein the transcription start site element comprises the nucleotide sequence [5′-GGGAAA-3′] set forth in SEQ ID NO: 53 or the nucleotide sequence [5′-AGGAAA-3′] set forth in SEQ ID NO: 54.
   E149. The mRNA of any one of embodiments E140-E148, wherein the structural RNA element is located about 45-50, about 40-45, about 35-40, about 30-35, about 25-30, about 20-25, about 15-20, about 10-15, about 5-10 nucleotides, about 1-5 nucleotide(s), or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) downstream of the C-rich RNA element in the 5′ UTR.
   E150. The mRNA of embodiment E149, wherein the structural RNA element is located about 40-45 nucleotides downstream of the C-rich RNA element in the 5′ UTR.
   E151. The mRNA of embodiment E149, wherein the structural RNA element is located about 35-40 nucleotides downstream of the C-rich RNA element in the 5′ UTR.
   E152. The mRNA of embodiment E149, wherein the structural RNA element is located about 30-35 nucleotides downstream of the C-rich RNA element in the 5′ UTR.
   E153. The mRNA of any one of embodiments E140-E152, wherein the GC-rich RNA element is located about 10-15, about 5-10, or about 1-5 nucleotides downstream of the structural RNA element in the 5′ UTR.
   E154. The mRNA of embodiment E153, wherein the GC-rich RNA element is located about 5, about 4, about 3, about 2, or 1 nucleotide downstream of the structural RNA element in the 5′ UTR.
   E155. The mRNA of any one of embodiments E140-E154, wherein the GC-rich RNA element is upstream of and immediately adjacent to the Kozak-like sequence in the 5′ UTR.
   E156. The mRNA of any one of the preceding embodiments, wherein the 5′ UTR comprises the nucleotide sequence of SEQ ID NO: 4, wherein a structural RNA element comprising a stem-loop is inserted, optionally wherein a GC-rich RNA element is inserted, optionally wherein a C-rich RNA element is inserted.
   E157. An mRNA comprising:
a 5′ cap;
a 5′ UTR comprising a structural RNA element comprising a stem-loop;
an ORF encoding a polypeptide; and
a 3′ UTR
wherein the structural RNA element comprises a nucleotide sequence at least 80%, 85%, 90%, 95%, 96%, 97%, 98%, 99% or 100% identical to the nucleotide sequence of SEQ ID NO: 6, wherein the 5′ UTR comprises the nucleotide sequence of SEQ ID NO: 4 or SEQ ID NO: 60 comprising a GC-rich RNA element comprising the sequence CCCCGGCGCC (SEQ ID NO: 1), and wherein the structural RNA element is inserted upstream of the GC-rich RNA element in the 5′ UTR.
   E158. An mRNA comprising:
a 5′ cap;
a 5′ UTR comprising a structural RNA element comprising a stem-loop;
an ORF encoding a polypeptide; and
a 3′ UTR,
wherein the structural RNA element comprises a sequence of 15-25 linked nucleotides comprising at least 60% G/C bases, wherein the structural RNA element comprises (i) a double-stranded stem of about 4-7 base pairs; (ii) a single-stranded loop of about 4-7 nucleotides; (iii) a nucleotide sequence which differs from SEQ ID NO: 6 by substitution, deletion or insertion of 1, 2, 3, 4, or 5 nucleotides; and (iv) a delta G (ΔG) of about −10 to −15 kcal/mol,
wherein the 5′ UTR comprises the nucleotide sequence of SEQ ID NO: 4 or SEQ ID NO: 60 comprising a GC-rich RNA element comprising the sequence CCCCGGCGCC (SEQ ID NO: 1), and wherein the structural RNA element is inserted upstream of the GC-rich RNA element in the 5′ UTR.
   E159. An mRNA comprising:
a 5′ cap;
a 5′ UTR comprising a structural RNA element comprising a stem-loop;
an ORF encoding a polypeptide; and
a 3′ UTR
wherein the structural RNA element comprises the nucleotide sequence of SEQ ID NO: 6, wherein the 5′ UTR comprises the nucleotide sequence of SEQ ID NO: 4 or SEQ ID NO: 60 comprising a GC-rich RNA element comprising the sequence CCCCGGCGCC (SEQ ID NO: 1), and wherein the structural RNA element is inserted upstream of the GC-rich RNA element in the 5′ UTR.
   E160. The mRNA of any one of embodiments E157-E159, wherein the structural RNA element is inserted about 1-5, 5-10, 10-20, 20-30, or 30-40 nucleotides, or about 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) upstream of the GC-rich RNA element in SEQ ID NO: 4 or SEQ ID NO: 60.
   E161. The mRNA of E160, wherein the structural RNA element is inserted 1-5 nucleotides upstream of the GC-rich RNA element in SEQ ID NO: 4 or SEQ ID NO: 60.
   E162. The mRNA of E160, wherein the structural RNA element is inserted 10-20 nucleotides upstream of the GC-rich RNA element in SEQ ID NO: 4 or SEQ ID NO: 60.
   E163. The mRNA of E160, wherein the structural RNA element is inserted 30-40 nucleotides upstream of the GC-rich RNA element in SEQ ID NO: 4 or SEQ ID NO: 60.
   E164. The mRNA of any one of embodiments E157-E159, wherein the structural RNA element is inserted upstream of and immediately adjacent to the GC-rich RNA element in SEQ ID NO: 4 or SEQ ID NO: 60.
   E165. The mRNA of any one of embodiments E157-E164, comprising a C-rich RNA element inserted proximal to the 5′ cap of the mRNA in SEQ ID NO: 4 or SEQ ID NO: 60, wherein the C-rich RNA element comprises a nucleotide sequence selected from selected from the group consisting of SEQ ID NO: 31, SEQ ID NO: 32 and SEQ ID NO: 33.
   E166. The mRNA of embodiment E165, wherein the C-rich RNA element comprises the nucleotide sequence of SEQ ID NO: 31.
   E167. The mRNA of embodiment any one of embodiments E165 or E166, wherein the C-rich RNA element is inserted about 1-10 nucleotides downstream of the 5′ cap in SEQ ID NO: 4 or SEQ ID NO: 60.
   E168. The mRNA of embodiment any one of embodiments E165 or E166, wherein the C-rich RNA element is inserted about 5-10 nucleotides downstream of the 5′ cap in SEQ ID NO: 4 or SEQ ID NO: 60.
   E169. The mRNA of embodiment any one of embodiments E165 or E166, wherein the C-rich RNA element is inserted about 1-6 nucleotides downstream of the 5′ cap of in SEQ ID NO: 4 or SEQ ID NO: 60.
   E170. The mRNA of any one of embodiments E165 or E166, wherein the C-rich RNA element is downstream of and immediately adjacent to a transcription start site element in the 5′UTR, wherein the transcription start site element comprises the nucleotide sequence [5′-GGGAAA-3′] in SEQ ID NO: 4 or the nucleotide sequence [5′-AGGAAA-3′] in SEQ ID NO: 60.
   E171. An mRNA comprising:
a 5′ cap;
a 5′ UTR;
an ORF encoding a polypeptide; and
a 3′ UTR
wherein the 5′ UTR comprises a nucleotide sequence selected from the group consisting of:
(i) the nucleotide sequence of SEQ ID NO: 116;
(ii) the nucleotide sequence of SEQ ID NO: 120;
(iii) the nucleotide sequence of SEQ ID NO: 124;
(iv) the nucleotide sequence of SEQ ID NO: 128; and
(v) the nucleotide sequence of SEQ ID NO: 41.
   E172. The mRNA of any one of the preceding embodiments, wherein the 3′UTR comprises a nucleotide sequence of a 3′UTR of a nuclear-encoded mitochondrially derived protein (NEMP).
   E173. The mRNA of embodiment E172, wherein binding of the 3′UTR to one or more RNA-binding proteins promotes the stabilization, localization, and/or translation of the mRNA.
   E174. The mRNA of any one of embodiments E172 or E173, wherein the NEMP is selected from the group consisting of: human OXAL1, human MRPS12, and mouse Sod2.
   E175. The mRNA of any one of embodiments E172-E174, wherein the nucleotide sequence of the 3′UTR is at least 70%, at least 75%, at least 80%, at least 85%, at least 90%, at least 95%, at least 96%, at least 97%, at least 98%, at least 99%, or 100% identical to the nucleotide sequence of the NEMP 3′UTR.
   E176. The mRNA of any one of embodiments E172-E174, wherein the 3′UTR differs from the nucleotide sequence of the NEMP 3′UTR by 1-5, 5-10, 10-15, 15-20, 20-25, 25-30, 30-35, 35-40, 40-45, 45-50 or about 50 or more nucleotides.
   E177. The mRNA of any one of embodiments E172-E176, wherein the 3′UTR is about 50-100 nucleotides, about 100-200 nucleotides, about 200-300 nucleotides, about 300-400 nucleotides, about 400-500 nucleotides, about 500-600, about 600-700 nucleotides, about 700-800 nucleotides, about 800-900 nucleotides, about 900-1000 nucleotides, about 1000-1100 nucleotides, about 1100-1200 nucleotides, about 1200-1300 nucleotides, about 1300-1400 nucleotides, or about 1400-1500 nucleotides in length.
   E178. The mRNA of any one of embodiments E1-E171, wherein the 3′ UTR comprises a nucleotide sequence at least 70%, at least 75%, at least 80%, at least 85%, at least 90%, at least 95%, at least 96%, at least 97%, at least 98%, at least 99% identical or 100% identical to a nucleotide sequence selected from the group consisting of: SEQ ID NO: 72, SEQ ID NO: 74; SEQ ID NO: 76; and SEQ ID NO: 78.
   E179. The mRNA of embodiment E178, wherein the 3′UTR comprises a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 72.
   E180. The mRNA of embodiment E178, wherein the 3′UTR comprises a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 74.
   E181. The mRNA of embodiment E178, wherein the 3′UTR comprises a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 76.
   E182. The mRNA of embodiment E178, wherein the 3′UTR comprises a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 78.
   E183. The mRNA of any one of embodiments E1-E171, wherein the 3′UTR differs from the NEMP 3′UTR by about 1-5, 5-10, 10-15, 15-20, 20-25, 25-30, 30-35, 35-40, 40-45, 45-50 or about 50-100 nucleotides, wherein the NEMP 3′UTR comprises a nucleotide sequence selected from the group consisting of: SEQ ID NO: 72, SEQ ID NO: 74; SEQ ID NO: 76; and SEQ ID NO: 78.
   E184. The mRNA of any one of embodiments E1-E171, wherein the 3′UTR comprises a nucleotide sequence selected from the group consisting of: SEQ ID NO: 72, SEQ ID NO: 74; SEQ ID NO: 76; and SEQ ID NO: 78.
   E185. The mRNA of embodiment E184, wherein the 3′UTR comprises the nucleotide sequence of SEQ ID NO: 72.
   E186. The mRNA of embodiment E184, wherein the 3′UTR comprises the nucleotide sequence of SEQ ID NO: 74.
   E187. The mRNA of embodiment E184, wherein the 3′UTR comprises the nucleotide sequence of SEQ ID NO: 76.
   E188. The mRNA of embodiment E184, wherein the 3′UTR comprises the nucleotide sequence of SEQ ID NO: 78.
   E189. The mRNA of any one of embodiments E171-E188, wherein the 3′ UTR comprises one or more microRNA (miRNA) binding sites.
   E190. The mRNA of embodiment E189, wherein the 3′UTR comprises 1, 2, 3, 4, 5, 6, 7, 8, 9, or 10 miRNA binding site(s).
   E191. The mRNA of embodiment E189, wherein the 3′UTR comprises 1, 2, 3 or 4 miRNA binding sites.
   E192. The mRNA of any one of embodiments E189-E191, wherein the miRNA binding site is targeted by miR-142-3p or miR-142-5p.
   E193. The mRNA of embodiment E192, wherein the miRNA binding site comprises a nucleotide sequence at least 80%, at least 85%, at least 90%, at least 95%, at least 96%, at least 97%, at least 98%, at least 99%, or 100% identical to the nucleotide sequence of SEQ ID NO: 179 or SEQ ID NO: 181.
   E194. The mRNA of embodiment E193, wherein the miRNA binding site comprises the nucleotide sequence of SEQ ID NO: 179.
   E195. The mRNA of embodiment E193, wherein the miRNA binding site comprises the nucleotide sequence of SEQ ID NO: 181.
   E196. The mRNA of any one of embodiments E189-E195, wherein the 3′UTR comprises one or more stop codons at the 5′end of the 3′UTR, and wherein the 3′UTR comprises 1, 2, 3, or 4 miRNA binding sites located proximal to the one or more stop codons.
   E197. The mRNA of embodiment E196, wherein the miRNA binding site(s) are located downstream of and immediately adjacent to the one or more stop codons at the 5′end of the 3′UTR.
   E198. The mRNA of embodiment E196, wherein the miRNA binding sites are located about 45-50, about 40-45, about 35-40, about 30-35, about 25-30, about 20-25, about 15-20, about 10-15, about 6-10 nucleotides, about 1-5 nucleotide(s), or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) downstream of the one or more stop codons at the 5′end of the 3′UTR.
   E199. The mRNA of embodiment E196, wherein the miRNA binding sites are located about 10, about 9, about 8, about 7, about 6, about 5, about 4, about 3, about 2, or about 1 nucleotide(s) downstream of the one or more stop codons at the 5′end of the 3′UTR.
   E200. The mRNA of any one of embodiments E189-E195, wherein the 3′UTR comprises 1, 2, 3, or 4 miRNA binding sites located proximal to the 3′end of the 3′UTR.
   E201. The mRNA of embodiment E200, wherein the miRNA binding site(s) are located upstream of and immediately adjacent to the 3′end of the 3′UTR.
   E202. The mRNA of embodiment E200, wherein the miRNA binding site(s) are located about 1-5, about 6-10, about 10-15, about 15-20, about 20-25, about 25-30, about 30-35, about 35-40, about 40-45, or about 45-50 nucleotide(s) or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotide(s) upstream of the 3′end of the 3′UTR.
   E203. The mRNA of embodiment E202, wherein the miRNA binding site(s) are located about 1, about 2, about 3, about 4, or about 5, about 6, about 7, about 8, about 9 or about 10 nucleotide(s) upstream of the 3′end of the 3′UTR.
   E204. The mRNA of any one of embodiments E196-E203, wherein the 3′UTR comprises 1, 2, 3, 4, 5, 6, 7, 8, 9, or 10 miRNA binding sites, wherein an upstream miRNA binding site is located directly adjacent to one or more downstream miRNA binding site(s).
   E205. The mRNA of any one of embodiments E196-E203, wherein the 3′UTR comprises 1, 2, 3, 4, 5, 6, 7, 8, 9, or 10 miRNA binding sites, wherein an upstream miRNA binding site is separated from a downstream miRNA binding site by about 1-5, about 1-10, about 5-10, about 5-15, about 10-20, about 15-20, about 15-30, or about 20-30 nucleotide(s) or about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotide(s).
   E206. The mRNA of any one of embodiments E196-E203, wherein the 3′UTR comprises 1, 2, 3, 4, 5, 6, 7, 8, 9, or 10 miRNA binding sites, wherein an upstream miRNA binding site is separated from a downstream miRNA binding site by about 1, about 2, about 3, about 4, about 5, about 6, about 7, about 8, about 9 or about 10 nucleotide(s).
   E207. The mRNA of any one of embodiments E1-E171, wherein the 3′ UTR comprises a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 78, wherein the 3′UTR comprises 1, 2, 3, or 4 miR-142-3p binding sites, and wherein the miR-142-3p binding site comprises the nucleotide sequence of SEQ ID NO: 179.
   E208. The mRNA of embodiment E207, wherein the 1, 2, 3, or 4 miR-142-3p binding sites are located proximal to the 3′end or the 3′UTR.
   E209. The mRNA of embodiment 208, wherein the 3′UTR comprises the nucleotide sequence of SEQ ID NO: 170.
   E210. The mRNA of embodiment E207, wherein the 3′UTR comprises one or more stop codons at the 5′end and wherein the 1, 2, 3, or 4 miR-142-3p binding sites are located proximal to the one or more stop codons.
   E211. The mRNA of embodiment E210, wherein the 3′UTR comprises the nucleotide sequence of SEQ ID NO: 172.
   E212. The mRNA of any one of embodiments E1-E171, wherein the 3′UTR comprises a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 76, wherein the 3′UTR comprises 1, 2, 3, or 4 miR-142-3p binding sites, and wherein the miR-142-3p binding site comprises the nucleotide sequence of SEQ ID NO: 179.
   E213. The mRNA of embodiment E212, wherein the 1, 2, 3, or 4 miR-142-3p binding sites are located proximal to the 3′end or the 3′UTR.
   E214. The mRNA of embodiment E212, wherein the 3′UTR comprises the nucleotide sequence of SEQ ID NO: 174.
   E215. The mRNA of embodiment E212, wherein the 3′UTR comprises one or more stop codons at the 5′end and wherein the 1, 2, 3, or 4 miR-142-3p binding sites are located proximal to the one or more stop codons.
   E216. The mRNA of embodiment E215, wherein the 3′UTR comprises the nucleotide sequence of SEQ ID NO: 176.
   E217. An mRNA comprising:
(i) a 5′ cap;
(ii) a 5′UTR;
(iii) an ORF encoding a polypeptide; and
(iv) a 3′UTR,
wherein the 3′UTR comprises a nucleotide sequence of a 3′UTR of a NEMP.
   E218. The mRNA of embodiment E217, wherein binding of the 3′UTR to one or more RNA-binding proteins promotes the stabilization, localization, and/or translation of the mRNA.
   E219. The mRNA of any one of embodiments E217 or E218, wherein the NEMP is selected from the group consisting of: human OXAL1, human MRPS12, and mouse Sod2.
   E220. The mRNA of any one of embodiments E217-E219, wherein the nucleotide sequence of the 3′UTR is at least 70%, at least 75%, at least 80%, at least 85%, at least 90%, at least 95%, at least 96%, at least 97%, at least 98%, at least 99%, or 100% identical to the nucleotide sequence of the NEMP 3′UTR.
   E221. The mRNA of any one of embodiments E217-E219, wherein the 3′UTR differs from the nucleotide sequence of the NEMP 3′UTR by 1-5, 5-10, 10-15, 15-20, 20-25, 25-30, 30-35, 35-40, 40-45, 45-50 or about 50 or more nucleotides.
   E222. The mRNA of any one of embodiments E217-E221, wherein the 3′UTR is about 50-100 nucleotides, about 100-200 nucleotides, about 200-300 nucleotides, about 300-400 nucleotides, about 400-500 nucleotides, about 500-600, about 600-700 nucleotides, about 700-800 nucleotides, about 800-900 nucleotides, about 900-1000 nucleotides, about 1000-1100 nucleotides, about 1100-1200 nucleotides, about 1200-1300 nucleotides, about 1300-1400 nucleotides, or about 1400-1500 nucleotides in length.
   E223. The mRNA of any one of embodiments E217-E219, wherein the 3′UTR comprises a nucleotide sequence at least 70%, at least 75%, at least 80%, at least 85%, at least 90%, at least 95%, at least 96%, at least 97%, at least 98%, at least 99% identical or 100% identical to a nucleotide sequence selected from the group consisting of: SEQ ID NO: 72; SEQ ID NO: 74; SEQ ID NO: 76; SEQ ID NO: 78; SEQ ID NO: 166; and SEQ ID NO: 167.
   E224. The mRNA of embodiment E223, wherein the 3′UTR comprises a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 72.
   E225. The mRNA of embodiment E223, wherein the 3′UTR comprises a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 74.
   E226. The mRNA of embodiment E223, wherein the 3′UTR comprises a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 76.
   E227. The mRNA of embodiment E223, wherein the 3′UTR comprises a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 78.
   E228. The mRNA of any one of embodiments E217-E219, wherein the 3′UTR differs from the NEMP 3′UTR by about 1-5, 5-10, 10-15, 15-20, 20-25, 25-30, 30-35, 35-40, 40-45, 45-50 or about 50-100 nucleotides, wherein the NEMP 3′UTR comprises a nucleotide sequence selected from the group consisting of: SEQ ID NO: 72; SEQ ID NO: 74; SEQ ID NO: 76; SEQ ID NO: 78; SEQ ID NO: 166; and SEQ ID NO: 167.
   E229. The mRNA of any one of embodiments E217-E219, wherein the 3′UTR comprises a nucleotide sequence selected from the group consisting of: SEQ ID NO: 72; SEQ ID NO: 74; SEQ ID NO: 76; SEQ ID NO: 78; SEQ ID NO: 166; and SEQ ID NO: 167.
   E230. The mRNA of embodiment E229, wherein the 3′UTR comprises the nucleotide sequence of SEQ ID NO: 72.
   E231. The mRNA of embodiment E229, wherein the 3′UTR comprises the nucleotide sequence of SEQ ID NO: 74.
   E232. The mRNA of embodiment E229, wherein the 3′UTR comprises the nucleotide sequence of SEQ ID NO: 76.
   E233. The mRNA of embodiment E229, wherein the 3′UTR comprises the nucleotide sequence of SEQ ID NO: 78.
   E234. The mRNA of any one of embodiments E217-E233, wherein the 3′UTR comprises one or more microRNA (miRNA) binding sites.
   E235. The mRNA of embodiment E234, wherein the 3′UTR comprises 1, 2, 3, 4, 5, 6, 7, 8, 9, or 10 miRNA binding site(s).
   E236. The mRNA of embodiment E234, wherein the 3′UTR comprises 1, 2, 3 or 4 miRNA binding sites.
   E237. The mRNA of any one of embodiments E234-E236, wherein the miRNA binding site is targeted by miR-142-3p or miR-142-5p.
   E238. The mRNA of embodiment E237, wherein the miRNA binding site comprises a nucleotide sequence at least 80%, at least 85%, at least 90%, at least 95%, at least 96%, at least 97%, at least 98%, at least 99%, or 100% identical to the nucleotide sequence of SEQ ID NO: 179 or SEQ ID NO: 181.
   E239. The mRNA of embodiment E238, wherein the miRNA binding site comprises the nucleotide sequence of SEQ ID NO: 179.
   E240. The mRNA of embodiment E238, wherein the miRNA binding site comprises the nucleotide sequence of SEQ ID NO: 181.
   E241. The mRNA of any one of embodiments E234-E240, wherein the 3′UTR comprises one or more stop codons at the 5′end of the 3′UTR, and wherein the 3′UTR comprises 1, 2, 3, or 4 miRNA binding sites located proximal to the one or more stop codons.
   E242. The mRNA of embodiment E241, wherein the miRNA binding site(s) are located downstream of and immediately adjacent to the one or more stop codons at the 5′end of the 3′UTR.
   E243. The mRNA of embodiment E241, wherein the miRNA binding sites are located about 45-50, about 40-45, about 35-40, about 30-35, about 25-30, about 20-25, about 15-20, about 10-15, about 6-10 nucleotides, about 1-5 nucleotide(s), or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) downstream of the one or more stop codons at the 5′end of the 3′UTR.
   E244. The mRNA of embodiment E241, wherein the miRNA binding sites are located about 10, about 9, about 8, about 7, about 6, about 5, about 4, about 3, about 2, or about 1 nucleotide(s) downstream of the one or more stop codons at the 5′end of the 3′UTR.
   E245. The mRNA of any one of embodiments E234-E240, wherein the 3′UTR comprises 1, 2, 3, or 4 miRNA binding sites located proximal to the 3′end of the 3′UTR.
   E246. The mRNA of embodiment E245, wherein the miRNA binding site(s) are located upstream of and immediately adjacent to the 3′end of the 3′UTR.
   E247. The mRNA of embodiment E245, wherein the miRNA binding site(s) are located about 1-5, about 6-10, about 10-15, about 15-20, about 20-25, about 25-30, about 30-35, about 35-40, about 40-45, or about 45-50 nucleotide(s) or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotide(s) upstream of the 3′end of the 3′UTR.
   E248. The mRNA of embodiment E245, wherein the miRNA binding site(s) are located about 1, about 2, about 3, about 4, or about 5, about 6, about 7, about 8, about 9 or about 10 nucleotide(s) upstream of the 3′end of the 3′UTR.
   E249. The mRNA of any one of embodiment E234-E248, wherein the 3′UTR comprises 1, 2, 3, 4, 5, 6, 7, 8, 9, or 10 miRNA binding sites, wherein an upstream miRNA binding site is located directly adjacent to one or more downstream miRNA binding site(s).
   E250. The mRNA of any one of embodiments E234-E248, wherein the 3′UTR comprises 1, 2, 3, 4, 5, 6, 7, 8, 9, or 10 miRNA binding sites, wherein an upstream miRNA binding site is separated from a downstream miRNA binding site by about 1-5, about 1-10, about 5-10, about 5-15, about 10-20, about 15-20, about 15-30, or about 20-30 nucleotide(s) or about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotide(s).
   E251. The mRNA of any one of embodiments E234-E248, wherein the 3′UTR comprises 1, 2, 3, 4, 5, 6, 7, 8, 9, or 10 miRNA binding sites, wherein an upstream miRNA binding site is separated from a downstream miRNA binding site by about 1, about 2, about 3, about 4, about 5, about 6, about 7, about 8, about 9 or about 10 nucleotide(s).
   E252. The mRNA of any one of embodiments E217-E218, wherein the 3′ UTR comprises a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 78, wherein the 3′UTR comprises 1, 2, 3, or 4 miR-142-3p binding sites, and wherein the miR-142-3p binding site comprises the nucleotide sequence of SEQ ID NO: 179.
   E253. The mRNA of embodiment E252, wherein the 1, 2, 3, or 4 miR-142-3p binding sites are located proximal to the 3′end or the 3′UTR.
   E254. The mRNA of embodiment E252, wherein the 3′UTR comprises the nucleotide sequence of SEQ ID NO: 170.
   E255. The mRNA of embodiment E252, wherein the 3′UTR comprises one or more stop codons at the 5′end and wherein the 1, 2, 3, or 4 miR-142-3p binding sites are located proximal to the one or more stop codons.
   E256. The mRNA of embodiment E252, wherein the 3′UTR comprises the nucleotide sequence of SEQ ID NO: 172.
   E257. The mRNA of any one of embodiments E217-E218, wherein the 3′UTR comprises a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 76, wherein the 3′UTR comprises 1, 2, 3, or 4 miR-142-3p binding sites, and wherein the miR-142-3p binding site comprises the nucleotide sequence of SEQ ID NO: 179.
   E258. The mRNA of embodiment EE257, wherein the 1, 2, 3, or 4 miR-142-3p binding sites are located proximal to the 3′end or the 3′UTR.
   E259. The mRNA of embodiment E257, wherein the 3′UTR comprises the nucleotide sequence of SEQ ID NO: 174.
   E260. The mRNA of embodiment EE257, wherein the 3′UTR comprises one or more stop codons at the 5′end and wherein the 1, 2, 3, or 4 miR-142-3p binding sites are located proximal to the one or more stop codons.
   E261. The mRNA of embodiment E257, wherein the 3′UTR comprises the nucleotide sequence of SEQ ID NO: 176.
   E262. An mRNA comprising:
a 5′ cap;
a 5′ UTR comprising a structural RNA element comprising a stem-loop, wherein the structural RNA element comprises a nucleotide sequence at least 80%, 85%, 90%, 95%, 96%, 97%, 98%, 99% or 100% identical to the nucleotide sequence of SEQ ID NO: 6;
an ORF encoding a polypeptide; and
a 3′ UTR, wherein the 3′UTR comprises a nucleotide sequence at least 70%, at least 75%, at least 80%, at least 85%, at least 90%, at least 95%, at least 96%, at least 97%, at least 98%, at least 99% identical or 100% identical to a nucleotide sequence of SEQ ID NO: 76; SEQ ID NO: 78; SEQ ID NO: 166; or SEQ ID NO: 167,
wherein the 5′ UTR comprises the nucleotide sequence of SEQ ID NO: 4 or SEQ ID NO: 60 comprising a GC-rich RNA element comprising the sequence CCCCGGCGCC (SEQ ID NO: 1), and wherein the structural RNA element is inserted upstream of the GC-rich RNA element in the 5′ UTR.
   E263. An mRNA comprising:
a 5′ cap;
a 5′ UTR comprising a structural RNA element comprising a stem-loop wherein the structural RNA element comprises a sequence of 15-25 linked nucleotides comprising at least 60% G/C bases, wherein the structural RNA element comprises (i) a double-stranded stem of about 4-7 base pairs; (ii) a single-stranded loop of about 4-7 nucleotides; (iii) a nucleotide sequence which differs from SEQ ID NO: 6 by substitution, deletion or insertion of 1, 2, 3, 4, or 5 nucleotides; and (iv) a delta G (ΔG) of about −10 to −15 kcal/mol;
an ORF encoding a polypeptide; and
a 3′ UTR, wherein the 3′UTR comprises a nucleotide sequence at least 70%, at least 75%, at least 80%, at least 85%, at least 90%, at least 95%, at least 96%, at least 97%, at least 98%, at least 99% identical or 100% identical to a nucleotide sequence of SEQ ID NO: 76; SEQ ID NO: 78; SEQ ID NO: 166; or SEQ ID NO: 167,
wherein the 5′ UTR comprises the nucleotide sequence of SEQ ID NO: 4 or SEQ ID NO: 60 comprising a GC-rich RNA element comprising the sequence CCCCGGCGCC (SEQ ID NO: 1), and wherein the structural RNA element is inserted upstream of the GC-rich RNA element in the 5′ UTR.
   E264. An mRNA comprising:
a 5′ cap;
a 5′ UTR comprising a structural RNA element comprising the nucleotide sequence of SEQ ID NO: 6;
an ORF encoding a polypeptide; and
a 3′ UTR, wherein the 3′UTR comprises a nucleotide sequence at least 70%, at least 75%, at least 80%, at least 85%, at least 90%, at least 95%, at least 96%, at least 97%, at least 98%, at least 99% identical or 100% identical to a nucleotide sequence of SEQ ID NO: 76; SEQ ID NO: 78; SEQ ID NO: 166; or SEQ ID NO: 167,
wherein the 5′ UTR comprises the nucleotide sequence of SEQ ID NO: 4 or SEQ ID NO: 60 comprising a GC-rich RNA element comprising the sequence CCCCGGCGCC (SEQ ID NO: 1), and wherein the structural RNA element is inserted upstream of the GC-rich RNA element in the 5′ UTR.
   E265. The mRNA of any one of embodiments E262-E264, wherein the structural RNA element is inserted about 1-5, 5-10, 10-20, 20-30, or 30-40 nucleotides, or about 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) upstream of the GC-rich RNA element in SEQ ID NO: 4 or SEQ ID NO: 60.
   E266. The mRNA of embodiment E265, wherein the structural RNA element is inserted 1-5 nucleotides upstream of the GC-rich RNA element in SEQ ID NO: 4 or SEQ ID NO: 60.
   E267. The mRNA of embodiment E265, wherein the structural RNA element is inserted 10-20 nucleotides upstream of the GC-rich RNA element in SEQ ID NO: 4 or SEQ ID NO: 60.
   E268. The mRNA of embodiment E265, wherein the structural RNA element is inserted 30-40 nucleotides upstream of the GC-rich RNA element in SEQ ID NO: 4 or SEQ ID NO: 60.
   E269. The mRNA of any one of embodiments E262-E264, wherein the structural RNA element is inserted upstream of and immediately adjacent to the GC-rich RNA element in SEQ ID NO: 4 or SEQ ID NO: 60.
   E270. The mRNA of any one of embodiments E262-E269, comprising a C-rich RNA element inserted proximal to the 5′ cap of the mRNA in SEQ ID NO: 4 or SEQ ID NO: 60, wherein the C-rich RNA element comprises a nucleotide sequence selected from selected from the group consisting of SEQ ID NO: 31, SEQ ID NO: 32 and SEQ ID NO: 33.
   E271. The mRNA of embodiment E270, wherein the C-rich RNA element comprises the nucleotide sequence of SEQ ID NO: 31.
   E272. The mRNA of embodiment any one of embodiments E270 or E271, wherein the C-rich RNA element is inserted about 1-10 nucleotides downstream of the 5′ cap in SEQ ID NO: 4 or SEQ ID NO: 60.
   E273. The mRNA of embodiment any one of embodiments E270 or E271, wherein the C-rich RNA element is inserted about 5-10 nucleotides downstream of the 5′ cap in SEQ ID NO: 4 or SEQ ID NO: 60.
   E274. The mRNA of embodiment any one of embodiments E270 or E271, wherein the C-rich RNA element is inserted about 1-6 nucleotides downstream of the 5′ cap of in SEQ ID NO: 4 or SEQ ID NO: 60.
   E275. The mRNA of any one of embodiments E270 or E271, wherein the C-rich RNA element is downstream of and immediately adjacent to a transcription start site element in the 5′UTR, wherein the transcription start site element comprises the nucleotide sequence [5′-GGGAAA-3′] in SEQ ID NO: 4 or the nucleotide sequence [5′-AGGAAA-3′] in SEQ ID NO: 60.
   E276. An mRNA comprising:
a 5′ cap;
a 5′ UTR, wherein the 5′ UTR comprises a nucleotide sequence selected from the group consisting of:
(i) the nucleotide sequence of SEQ ID NO: 116;
(ii) the nucleotide sequence of SEQ ID NO: 120;
(iii) the nucleotide sequence of SEQ ID NO: 124;
(iv) the nucleotide sequence of SEQ ID NO: 41; and
(v) the nucleotide sequence of SEQ ID NO: 128;
an ORF encoding a polypeptide; and
a 3′ UTR, wherein the 3′UTR comprises a nucleotide sequence at least 70%, at least 75%, at least 80%, at least 85%, at least 90%, at least 95%, at least 96%, at least 97%, at least 98%, at least 99% identical or 100% identical to a nucleotide sequence of SEQ ID NO: 76; SEQ ID NO: 78; SEQ ID NO: 166; and SEQ ID NO: 167.
   E277. The mRNA of embodiment E276, wherein the 3′UTR comprises one or more microRNA (miRNA) binding sites.
   E278. The mRNA of embodiment E277, wherein the 3′UTR comprises 1, 2, 3, 4, 5, 6, 7, 8, 9, or 10 miRNA binding site(s).
   E279. The mRNA of embodiment E277, wherein the 3′UTR comprises 1, 2, 3 or 4 miRNA binding sites.
   E280. The mRNA of any one of embodiments E277-E279, wherein the miRNA binding site is targeted by miR-142-3p or miR-142-5p.
   E281. The mRNA of embodiment E280, wherein the miRNA binding site comprises a nucleotide sequence at least 80%, at least 85%, at least 90%, at least 95%, at least 96%, at least 97%, at least 98%, at least 99%, or 100% identical to the nucleotide sequence of SEQ ID NO: 179 or SEQ ID NO: 181.
   E282. The mRNA of embodiment E281, wherein the miRNA binding site comprises the nucleotide sequence of SEQ ID NO: 179.
   E283. The mRNA of embodiment E281, wherein the miRNA binding site comprises the nucleotide sequence of SEQ ID NO: 181.
   E284. The mRNA of any one of embodiments E276-E283, wherein the 3′UTR comprises one or more stop codons at the 5′end of the 3′UTR, and wherein the 3′UTR comprises 1, 2, 3, or 4 miRNA binding sites located proximal to the one or more stop codons.
   E285. The mRNA of embodiment E284, wherein the miRNA binding site(s) are located downstream of and immediately adjacent to the one or more stop codons at the 5′end of the 3′UTR.
   E286. The mRNA of embodiment E284, wherein the miRNA binding sites are located about 45-50, about 40-45, about 35-40, about 30-35, about 25-30, about 20-25, about 15-20, about 10-15, about 6-10 nucleotides, about 1-5 nucleotide(s), or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) downstream of the one or more stop codons at the 5′end of the 3′UTR.
   E287. The mRNA of embodiment E286, wherein the miRNA binding sites are located about 10, about 9, about 8, about 7, about 6, about 5, about 4, about 3, about 2, or about 1 nucleotide(s) downstream of the one or more stop codons at the 5′end of the 3′UTR.
   E288. The mRNA of any one of embodiments E277-E283, wherein the 3′UTR comprises 1, 2, 3, or 4 miRNA binding sites located proximal to the 3′end of the 3′UTR.
   E289. The mRNA of embodiment E288, wherein the miRNA binding site(s) are located upstream of and immediately adjacent to the 3′end of the 3′UTR.
   E290. The mRNA of embodiment E289, wherein the miRNA binding site(s) are located about 1-5, about 6-10, about 10-15, about 15-20, about 20-25, about 25-30, about 30-35, about 35-40, about 40-45, or about 45-50 nucleotide(s) or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotide(s) upstream of the 3′end of the 3′UTR.
   E291. The mRNA of embodiment E289, wherein the miRNA binding site(s) are located about 1, about 2, about 3, about 4, or about 5, about 6, about 7, about 8, about 9 or about 10 nucleotide(s) upstream of the 3′end of the 3′UTR.
   E292. The mRNA of any one of embodiments E288-E291, wherein the 3′UTR comprises 1, 2, 3, 4, 5, 6, 7, 8, 9, or 10 miRNA binding sites, wherein an upstream miRNA binding site is located directly adjacent to one or more downstream miRNA binding site(s).
   E293. The mRNA of any one of embodiments E280-E292, wherein the 3′UTR comprises 1, 2, 3, 4, 5, 6, 7, 8, 9, or 10 miRNA binding sites, wherein an upstream miRNA binding site is separated from a downstream miRNA binding site by about 1-5, about 1-10, about 5-10, about 5-15, about 10-20, about 15-20, about 15-30, or about 20-30 nucleotide(s) or about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotide(s).
   E294. The mRNA of any one of embodiments E280-E292, wherein the 3′UTR comprises 1, 2, 3, 4, 5, 6, 7, 8, 9, or 10 miRNA binding sites, wherein an upstream miRNA binding site is separated from a downstream miRNA binding site by about 1, about 2, about 3, about 4, about 5, about 6, about 7, about 8, about 9 or about 10 nucleotide(s).
   E295. The mRNA of embodiment E276, wherein the 3′ UTR comprises a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 78, wherein the 3′UTR comprises 1, 2, 3, or 4 miR-142-3p binding sites, and wherein the miR-142-3p binding site comprises the nucleotide sequence of SEQ ID NO: 179.
   E296. The mRNA of embodiment E295, wherein the 1, 2, 3, or 4 miR-142-3p binding sites are located proximal to the 3′end or the 3′UTR.
   E297. The mRNA of embodiment E296, wherein the 3′UTR comprises the nucleotide sequence of SEQ ID NO: 170.
   E298. The mRNA of embodiment E295, wherein the 3′UTR comprises one or more stop codons at the 5′end and wherein the 1, 2, 3, or 4 miR-142-3p binding sites are located proximal to the one or more stop codons.
   E299. The mRNA of embodiment E298, wherein the 3′UTR comprises the nucleotide sequence of SEQ ID NO: 172.
   E300. The mRNA of embodiment E276, wherein the 3′UTR comprises a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 76, wherein the 3′UTR comprises 1, 2, 3, or 4 miR-142-3p binding sites, and wherein the miR-142-3p binding site comprises the nucleotide sequence of SEQ ID NO: 179.
   E301. The mRNA of embodiment E300, wherein the 1, 2, 3, or 4 miR-142-3p binding sites are located proximal to the 3′end or the 3′UTR.
   E302. The mRNA of embodiment E301, wherein the 3′UTR comprises the nucleotide sequence of SEQ ID NO: 174.
   E303. The mRNA of embodiment E300, wherein the 3′UTR comprises one or more stop codons at the 5′end and wherein the 1, 2, 3, or 4 miR-142-3p binding sites are located proximal to the one or more stop codons.
   E304. The mRNA of embodiment E303, wherein the 3′UTR comprises the nucleotide sequence of SEQ ID NO: 176.
   E305. An mRNA comprising
a 5′ cap;
a 5′ UTR, wherein the 5′ UTR comprises a nucleotide sequence selected from the group consisting of:
(i) the nucleotide sequence of SEQ ID NO: 116;
(ii) the nucleotide sequence of SEQ ID NO: 120;
(iii) the nucleotide sequence of SEQ ID NO: 124;
(iv) the nucleotide sequence of SEQ ID NO: 41; and
(v) the nucleotide sequence of SEQ ID NO: 128;
an ORF encoding a polypeptide; and
a 3′ UTR, wherein the 3′UTR comprises a nucleotide sequence selected from the group consisting of:
(i) the nucleotide sequence of SEQ ID NO: 170,
(ii) the nucleotide sequence of SEQ ID NO: 172,
(iii) the nucleotide sequence of SEQ ID NO: 174; and
(iv) the nucleotide sequence of SEQ ID NO: 176.
   E306. An mRNA comprising
a 5′ cap;
a 5′ UTR;
an ORF encoding a polypeptide; and
a 3′ UTR,
wherein the 5′ UTR and 3′ UTR are selected from the group consisting of:
(i) the nucleotide sequence of SEQ ID NO: 120 and the nucleotide sequence of SEQ ID NO: 170;
(ii) the nucleotide sequence of SEQ ID NO: 120 and the nucleotide sequence of SEQ ID NO: 172;
(iii) the nucleotide sequence of SEQ ID NO: 120 and the nucleotide sequence of SEQ ID NO: 174;
(iv) the nucleotide sequence of SEQ ID NO: 120 and the nucleotide sequence of SEQ ID NO: 176;
(v) the nucleotide sequence of SEQ ID NO: 41 and the nucleotide sequence of SEQ ID NO: 170;
(vi) the nucleotide sequence of SEQ ID NO: 41 and the nucleotide sequence of SEQ ID NO: 172;
(vii) the nucleotide sequence of SEQ ID NO: 41 and the nucleotide sequence of SEQ ID NO: 174;
(viii) the nucleotide sequence of SEQ ID NO: 41 and the nucleotide sequence of SEQ ID NO: 176;
(ix) the nucleotide sequence of SEQ ID NO: 128 and the nucleotide sequence of SEQ ID NO: 170;
(x) the nucleotide sequence of SEQ ID NO: 128 and the nucleotide sequence of SEQ ID NO: 172;
(xi) the nucleotide sequence of SEQ ID NO: 128 and the nucleotide sequence of SEQ ID NO: 174; and
(xii) the nucleotide sequence of SEQ ID NO: 128 and the nucleotide sequence of SEQ ID NO: 176.
   E307. The mRNA of embodiment E306, wherein the 5′ UTR and 3′ UTR are selected from the group consisting of:
(i) the nucleotide sequence of SEQ ID NO: 120 and the nucleotide sequence of SEQ ID NO: 170; and
(ii) the nucleotide sequence of SEQ ID NO: 120 and the nucleotide sequence of SEQ ID NO: 172.
   E308. The mRNA of any one of the preceding embodiments, wherein the mRNA comprises at least one chemically modified nucleoside, and/or wherein the mRNA comprises at least one endonuclease sensitive sequence motif, wherein the endonuclease sensitive sequence motif comprises the nucleotide sequence WGA, wherein W=adenine (A) or uracil (U), and wherein the at least one endonuclease sensitive sequence motif is altered by substitution or deletion, thereby increasing mRNA stability, increasing mRNA half-life, and/or decreasing resistance or susceptibility of the mRNA to endonuclease activity.
   E309. The mRNA of embodiment E308, wherein the at least one chemically modified nucleoside is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.
   E310. The mRNA of any one of embodiments E308-E309, wherein least about 10%, at least about 20%, at least about 30%, at least about 40%, at least about 50%, at least about 60%, at least about 70%, at least about 80%, at least about 90%, at least about 95%, at least about 99%, or about 100% of the nucleosides comprising the mRNA comprise the at least one chemically modified nucleoside.
   E311. The mRNA of any one of embodiments E308-E310, wherein the at least one chemically modified nucleoside is N1-methylpseudouridine, and wherein at least 80%, at least 85%, at least 90%, at least 95%, at least 99%, or 100% of the uracil nucleotides are N1-methylpseudouridine.
   E312. The mRNA of embodiment E311, wherein the mRNA is fully modified with N1-methylpseudouridine.
   E313. The mRNA of any one of embodiments E308-E310, wherein the at least one modified nucleoside is 5-methoxyuridine.
   E314. The mRNA of embodiment E313, wherein at least 95% of uracil nucleotides comprising the ORF comprise 5-methoxyuridine, and wherein the uracil content in the ORF is between about 100% and about 150% of the theoretical minimum.
   E315. The mRNA of embodiment E314, wherein the mRNA is fully modified with 5-methoxyuridine.
   E316. The mRNA of any one of the preceding embodiments comprising a poly A tail.
   E317. The mRNA of any one of the preceding embodiments, wherein the mRNA comprises a 5′Cap 1 structure.
   E318. The mRNA of any one of the preceding embodiments, wherein an expression level and/or an activity of the polypeptide translated from the mRNA is increased relative to an mRNA that does not comprise the 5′ UTR, 3′ UTR, or a combination thereof.
   E319. A pharmaceutical composition comprising the mRNA of any one of the preceding embodiments and a pharmaceutically acceptable carrier.
   E320. A lipid nanoparticle comprising the mRNA of any one of embodiments E1-E318.
   E321. The lipid nanoparticle of embodiment E320, wherein the lipid nanoparticle comprises an ionizable lipid, a sterol, a phospholipid, and a polyethylene glycol lipid.
   E322. A pharmaceutical composition comprising the lipid nanoparticle of embodiments E320 or E321, and a pharmaceutically acceptable carrier.
   E323. The mRNA of any one of embodiments E1-E318, the pharmaceutical composition of any one of embodiments E319 or E322, or the lipid nanoparticle of embodiments E320 or E321, for use in treating or delaying progression of a disease or disorder in a subject in need thereof.
   E324. Use of the mRNA of any one of embodiments E1-EE318, the pharmaceutical composition of any one of embodiments E319 or E322, or the lipid nanoparticle of embodiments E320 or E321, in the manufacture of a medicament for treating or delaying progression of a disease or disorder in a subject in need thereof.
   E325. A kit comprising a container comprising the mRNA of any one of embodiments E1-E318, the pharmaceutical composition of any one of embodiments E319 or E322, or the lipid nanoparticle of embodiments E320 or E321, and a package insert comprising instructions for administration of the mRNA, the pharmaceutical composition of lipid nanoparticle, for treating or delaying progression of a disease or disorder in a subject.
   E326. A method of treating or delaying progression of a disease or disorder in a subject in need thereof, the method comprising administering the mRNA according to any one of embodiments E1-E318, the pharmaceutical composition according to embodiments E319 or E322, or the lipid nanoparticle according to embodiments E320 or E321, thereby treating or delaying progression of the disease or disorder in the subject.

### EXAMPLES

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the disclosure.

### Materials & Methods

Synthesis of mRNA. mRNAs were synthesized in vitro from linearized DNA templates which include the 5′ UTR, 3′UTR and polyA tail, followed by addition of a 5′ CAP.

### Example 1: Increased Expression and Activity of an Enzyme Translated from mRNA Comprising an RNAse P stem loop in the 5′UTR

An RNA element was incorporated into the 5′UTR of a reporter mRNA to assess its effect on mRNA expression and activity of translated protein. The RNA element that was evaluated was a stem-loop structure derived from the RNA component of the human RNAse P ribonucleoprotein (referred to as “RNAse P stem loop”). The reporter mRNA encoded a human cellular enzyme (referred to as enzyme_A), and the effect of a 5′UTR RNAse P stem loop was evaluated on enzyme_A expression and activity. The reporter mRNAs were prepared by in vitro-transcription and fully modified with N1-methyl pseudouridine (m¹ψ) in place of uracil. The mRNAs comprised a reference 3′UTR (3′v1.1, SEQ ID NO: 70).

The RNAse P stem loop comprised a nucleotide sequence identified by SEQ ID NO: 6. An RNAse P stem loop was incorporated into a reference 5′UTR identified by SEQ ID NO: 4 (5′v1.1). The reference 5′UTR comprised a GC-rich RNA element (SEQ ID NO: 1) that was positioned near the Kozak-like sequence. Incorporation of a GC-rich RNA element has been shown to improve mRNA expression (PCT Application No. PCT/US2018/033519, filed May 18, 2018, the entire contents of which are expressly incorporated herein by reference).

Three reporter mRNA constructs were prepared, wherein the location of the RNAse P stem loop in the 5′UTR was varied. The mRNAs tested comprised 5′UTRs as shown in Table 15.

TABLE 15
Exemplary 5′UTRs comprising RNA Elements

| | | | SEQ |
|---|---|---|---|
| Identifier | Name | Sequence | ID NO |
| RNA | V1 | CCCCGGCGCC | 1 |
| element | | | |
| RNA | RNAse P stem | UCUCCCUGAGCUUCAGGGAG | 6 |
| element | loop | | |
| 5′UTR | 5′v1.0 | GGGAAAUAAGAGAGAAAAGAAGAGUAAGAAGAAA | 45 |
| | | UAUAAGAGCCACC | |
| 5′UTR | 5′v1.1 | GGGAAAUAAGAGAGAAAAGAAGAGUAAGAAGAAA | 4 |
| | | UAUAAGACCCCGGCGCCGCCACC | |
| 5′UTR | RNAseP_p1 | GGGAAAUAAGAGAGAAAAGAAGAGUAAGAAGAAA | 116 |
| | | UAUAAGAUCUCCCUGAGCUUCAGGGAGCCCCGGC | |
| | | GCCGCCACC | |
| 5′UTR | RNAseP_p2 | GGGAAAUCUCCCUGAGCUUCAGGGAGUAAGAGAG | 120 |
| | | AAAAGAAGAGUAAGAAGAAAUAUAAGACCCCGGCG | |
| | | CCGCCACC | |
| 5′UTR | RNAseP_p3 | GGGAAAUAAGAGAGAAAAGAAGAUCUCCCUGAGC | 124 |
| | | UUCAGGGAGGUAAGAAGAAAUAUAAGACCCCGGCG | |
| | | CCGCCACC | |

Sequence annotation: GC-rich RNA element (italics); RNAse P stem loop (bold)

The effect of a 5′UTR RNAse P stem loop on mRNA expression and activity of translated enzyme_A protein was evaluated in hepatocytes isolated from mice that were deficient in enzyme_A. Hepatocytes were isolated and transfected with 0.5 μg of mRNA using Lipofectamine® Messenger Max™ (ThermoFisher Scientific). Cells were lysed at 24 hours following transfection and lysates were tested for enzyme_A expression by capillary electrophoresis and activity by an in house developed assay to measure the product of enzyme_A activity (referred to as a first biomarker of enzyme_A or enzyme_A-BM1) that is expected to accumulate with enzyme_A activity.

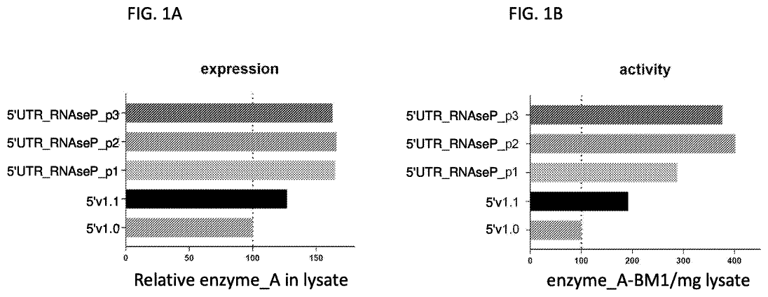

The expression level of enzyme_A measured in vitro is shown in FIG. 1A. Expression was higher for mRNAs comprising a 5′UTR RNAse P stem loop relative to the reference 5′UTRs. The expression level was similar for all mRNAs with an RNAse P stem loop, indicating that the location of the RNAse P stem loop does not have an effect on mRNA translation. The measured enzymatic activity showed a similar trend (FIG. 1B). Enzymatic activity was higher for mRNAs with a RNAse P stem loop compared to the reference mRNAs.

Thus, incorporation of an RNAse P stem loop in the 5′UTR results in increased mRNA expression and activity of an encoded enzyme. Additionally, the location of the RNAse P stem loop in the 5′UTR does not affect its functionality.

### Example 2: An RNAse P Stem Loop in the 5′UTR Yields Increased Translation Fidelity

One potential mechanism for increased mRNA translation is that RNA elements in the 5′UTR improve initiation fidelity. The initiation fidelity of mRNA translation is affected by the rate of leaky scanning. Leaky scanning refers to an event wherein the ribosome bypasses the desired initiation codon that begins the open reading frame encoding a desired translation product (ORE). Leaky scanning results in a polypeptide product that is either out-of-frame or truncated. High levels of leaky scanning contribute to poor expression of an encoded polypeptide. Thus, RNA elements in the 5′UTR that reduce the level of leaky scanning can result in higher levels of mRNA expression.

The effect of an RNAse P stem loop in the 5′UTR was evaluated for its effect on mRNA expression level and initiation fidelity (e.g., leaky scanning). A C-rich RNA element alone or in combination with a GC-rich RNA element has been shown to decrease leaky scanning (PCT Application No. PCT/US2019/027089, filed Apr. 11, 2019 the entire contents of which are expressly incorporated herein by reference). To determine if inclusion of an RNAse P stem loop would impact initiation fidelity, combinations of an RNAse P stem loop, a C-rich RNA element, and a GC-rich RNA element were evaluated for their effect on the rate of leaky scanning. The 5′UTRs that were evaluated are identified by Table 16.

TABLE 16
Exemplary 5′UTRs comprising RNA elements

| | Name | | SEQ |
|---|---|---|---|
| Identifier | (Origin) | Sequence | ID NO |
| RNA | V1 | CCCCGGCGCC | 1 |
| element | | | |
| RNA | GCC | CGCC | 52 |
| element | | | |
| RNA | CR3 | CCCCCCACCCCC | 31 |
| element | | | |
| RNA | CR5 | CCCCACAACC | 33 |
| element | | | |
| RNA | RNAse P stem | UCUCCCUGAGCUUCAGGGAG | 6 |
| element | loop (RNAseP) | | |
| 5′UTR | 5′v1.1 | GGGAAAUAAGAGAGAAAAGAAGAGUAAGAAGAAA | 4 |
| | | UAUAAGACCCCGGCGCCGCCACC | |
| 5′UTR | RNAseP_p1 | GGGAAAUAAGAGAGAAAAGAAGAGUAAGAAGAAA | 116 |
| | (5′v1.1 + | UAUAAGAUCUCCCUGAGCUUCAGGGAGCCCCGGC | |
| | RNAseP) | GCCGCCACC | |
| 5′UTR | F593 | GGGAAACCCCCCACCCCCGUAAGAGAGAAAAGAAG | 128 |
| | (5′v1.1 + | AGUAAGAAGAAAUAUAAGAUCUCCCUGAGCUUCA | |
| | RNAseP + CR3) | GGGAGCCCCGGCGCCGCCACC | |
| 5′UTR | F153 | GGGAAAUCCCCACAACCGCCUCAUAUCCAGGCUCA | 41 |
| | (Viral5′UTR + | AGAAUAGAGCUCAGUGUUUUGUUGUUUAAUCAUUC | |
| | GCC + CR5) | CGACGUGUUUUGCGAUAUUCGCGCAAAGCAGCCAG | |
| | | UCGCGCGCUUGCUUUUAAGUAGAGUUGUUUUUCCA | |
| | | CCCGUUUGCCAGGCAUCUUUAAUUUAACAUAUUUU | |
| | | UAUUUUUCAGGCUAACCUACGCCGCCACC | |

Sequence annotation: GC-rich RNA element (italics); RNAse P stem loop (bold); C-rich RNA element (underline)

To evaluate initiation fidelity (leaky scanning), a 3xFLAG reporter system was used to quantify the percentage of translated protein that is derived from leaky scanning. Specifically, reporter mRNAs were designed such that (i) translation initiation from an initial AUG start codon would produce an eGFP polypeptide fused to an N-terminal 3xFLAG epitope tag and (ii) translation initiation from a downstream AUG start codon would produce eGFP protein only without an epitope tag. The ratio of epitope-tagged eGFP to eGFP lacking an epitope tag provides a measure of initiation at the first start codon (e.g., translation fidelity) relative to initiation at the second start codon (e.g., leaky scanning).

Reporter mRNAs encoding a 3xFLAG tag starting with a first AUG initiation codon and eGFP starting with a second AUG initiation codon were generated with the 5′UTRs described above. Reporter mRNAs were transfected into HeLa cells and AML12 cells using Lipofectamine 2000. Lysates were generated from cells at six hours post-transfection and abundance of protein products was measured using a quantitative chemiluminescence immunoassay (ProteinSimple® Wes® Simple Western™, San Jose, Calif.) using an anti-GFP antibody. The amount of leaky scanning was calculated by determining the band intensity corresponding to the truncated eGFP polypeptide produced by leaky scanning (short band) and the band intensity corresponding to eGFP polypeptide produced from the full-length 3xFLAG-eGFP polypeptide (long band) and dividing the short band intensity by the sum of the short band intensity and long band intensity (leaky scanning rate=short band/(short band+long band)). The leaky scanning for reporter mRNAs comprising 5′UTRs with RNA elements was normalized to the leaky scanning for a reporter mRNA comprising a reference 5′UTR (5′v1.1; SEQ ID NO: 4) which was given a value of 1.0.

The effect of RNA elements in the 5′UTR on mRNA expression level was also determined. mRNAs encoding eGFP fused to a degron on the C-terminus (deg-GFP) were prepared with 5′UTRs comprising combinations of RNA elements as described above. The mRNAs were transfected into HeLa cells and AML12 cells using Lipofectamine 2000. Expression levels of the mRNA reporter constructs encoding deg-GFP were determined by measuring the total fluorescence of cells after 48 h using an IncuCyte® S3 Live-Cell Analysis System (Essen BioScience, Ann Arbor, Mich.). The amount of total fluorescence produced by deg-GFP polypeptide is proportional to the expression level of the deg-GFP polypeptide encoded by the mRNA reporter constructs and is provided in arbitrary units. Expression level was normalized to the expression level generated by a reporter mRNA comprising a reference 5′UTR (5′v1.1; SEQ ID NO: 4).

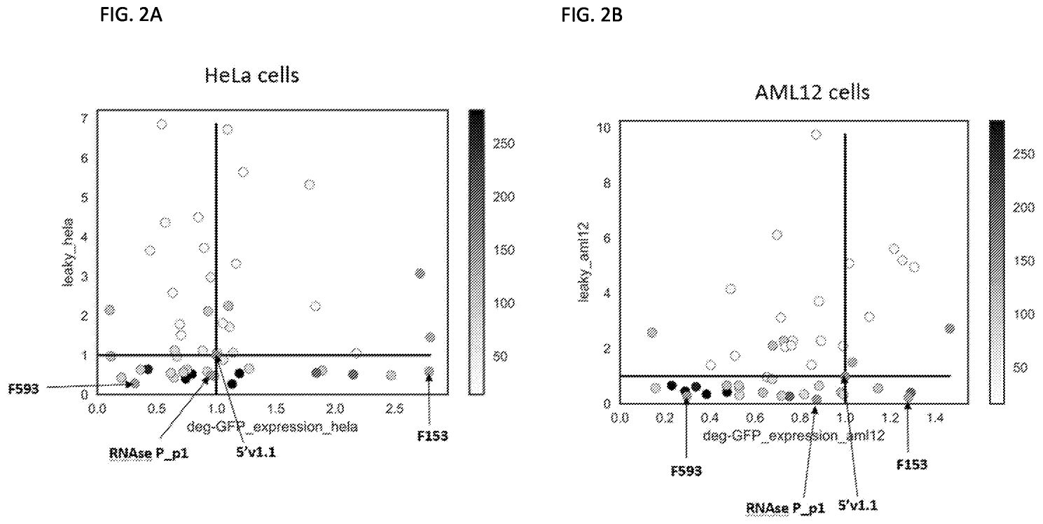

FIGS. 2A-2B show leaky scanning plotted against expression level for each reporter mRNA construct described above, showing that in both HeLa and AML12 cells, these variables were uncorrelated. One mRNA with a RNAse P stem loop, F593 5′UTR, showed a decrease in both expression and leaky scanning relative to the reference mRNA. While the other mRNA with an RNAse P stem loop, RNAseP_p 1 5′UTR, also showed a decrease in leaky scanning but had a similar expression to the reporter mRNA. The mRNA reporter construct with a F153 5′UTR showed a decrease in leaky scanning and a higher expression level compared to the reference mRNA. These effects were similar in HeLa cells (FIG. 2A) and in AML12 cells (FIG. 2B). Values for the initiation fidelity and mRNA expression level normalized to the reference 5′UTR are shown in Table 17.

These results demonstrate that the rate of leaky scanning is reduced for mRNAs with a 5′UTR comprising an RNAse P stem loop either in combination with a GC-rich RNA element (RNAseP_p1) or in combination with both a GC-rich RNA element and a C-rich RNA element (F593).

TABLE 17
Effect of 5′UTR RNA elements on mRNA expression
and leaky scanning in HeLa and AML12 cells

| | SEQ | | Relative | | Relative leaky | |
|---|---|---|---|---|---|---|
| mRNA | ID | | expression | | scanning | |
| 5′UTR | NO | Length | HeLa | AML12 | Aml12 | HeLa |
| F593 | 128 | 90 | 0.31 | 0.30 | 0.28 | 0.28 |
| F153 | 41 | 204 | 2.2 | 1.28 | 0.23 | 0.59 |
| RNAseP_p1 | 116 | 77 | 0.95 | 0.87 | 0.15 | 0.49 |
| 5′v1.1 | 4 | 57 | 1.00 | 1.00 | 1.00 | 1.00 |

Additionally, the length of the 5′UTR was varied and the effect on the rate of leaky scanning of a reporter mRNA was evaluated in both HeLa cells (FIG. 2A) and AML12 cells (FIG. 2B). As shown in FIGS. 2A-B, the rate of leaky scanning is plotted against expression level. The length of the 5′UTR is indicated by the color shading of the data point, wherein a short 5′UTR with length <50 nucleotides is shaded in white and long 5′UTRs with length >250 nucleotides is shaded in black. For both HeLa cells and AM12 cells, a short 5′UTR gave high levels of leaky scanning relative to the reference 5′UTR, while a long 5′UTR gave low levels of leaky scanning. These results demonstrate that the length of the 5′ UTR is inversely correlated with the rate of leaky scanning.

These results demonstrate than an mRNA with a 5′UTR comprising an RNAse P stem loop in combination with a GC-rich RNA element or in combination with both a GC-rich RNA element and a C-rich RNA element exhibits less leaky scanning relative to an mRNA that does not comprise this element. Further, these results demonstrate that leaky scanning and the length of the 5′UTR are correlated.

### Example 3: Identification of 3′UTRs that Provide Increased In Vitro mRNA Expression and Activity of an Encoded Cellular Enzyme

The 3′UTR was also optimized for increased mRNA expression and activity of translated protein. Reporter mRNAs encoded enzyme_A and comprised reference 5′UTRs (5′v1.1, SEQ ID NO: 4 or 5′v1.0, SEQ ID NO: 45). The reporter mRNAs were prepared by in vitro-transcription and fully modified with N1-methyl pseudouridine (mhWi) in place of uracil.

The 3′UTRs that were evaluated included 3′UTRs derived from genes encoding nuclear-encoded mitochondrial proteins: a 3′UTR derived from the human MRPS12 gene (referred to as Rps12 3′UTR), a 3′UTR derived from the mouse Sod2 gene (referred to as Sod2 3′UTR), or a 3′UTR derived from the human OXA1L gene (referred to as Oxal 3′UTR). These were compared to a reference 3′UTR (3′v1.1). Sequence identifiers for the 3′UTRs evaluated are shown in Table 18.

TABLE 18
Exemplary 3′UTRs

| Identifier | Name | Origin | SEQ ID NO |
|---|---|---|---|
| 3′UTR | 3′v1.1 | — | 70 |
| 3′UTR | rps12 3′UTR | Human MRPS12 | 76 |
| 3′UTR | Sod2 3′UTR | Mouse Sod2 | 78 |
| 3′UTR | Oxa1 3′UTR | Human OXA1L | 74 |

Expression and activity of translated protein was evaluated in vitro using hepatocytes from mice deficient in enzyme_A as described in Example 1. Cells were transfected with 0.5 μg of mRNA and were lysed 24 hours following transfection. The lysates were tested for enzyme_A expression by capillary electrophoresis and activity of enzyme_A by measuring the product of enzyme_A activity (e.g., referred to as a first biomarker of enzyme_A or enzyme_A-BM1) using HPLC.

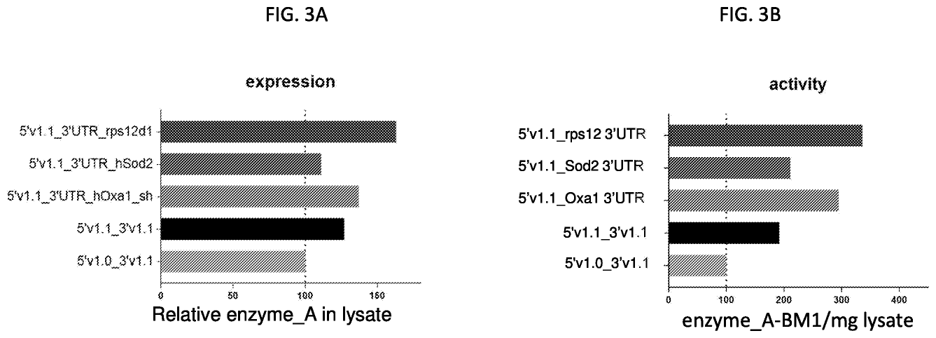

As shown in FIG. 3A, the Rps12 and Oxal 3′UTRs resulted in increased enzyme_A expression relative to a reference 3′UTR (3′v1.1). The highest expression was seen for a rps12 3′UTR. Additionally, as shown in FIG. 3B, the rps12, Sod2, and Oxal 3′UTRs also gave increased enzyme_A activity relative to both reference 3′UTRs, with enzymatic activity indicated by the amount of enzyme_A-BM1 produced per mg of hepatocyte lysate. Both the rps12 3′UTR and the Oxal 3′UTR resulted in high levels of enzyme_A activity.

### Example 4: Evaluation of 3′UTRs for Increased mRNA Potency In Vivo

The effect of 3′UTRs described in Example 3 on the expression level and activity of a cellular enzyme translated from mRNAs was evaluated in vivo. The mRNAs encoded a cellular enzyme (referred to as enzyme_B) and were prepared by in vitro-transcription. Additionally, the mRNAs were altered to minimize uracil content and fully modified to replace remaining uracil with 5-methoxy uridine (mo⁵U).

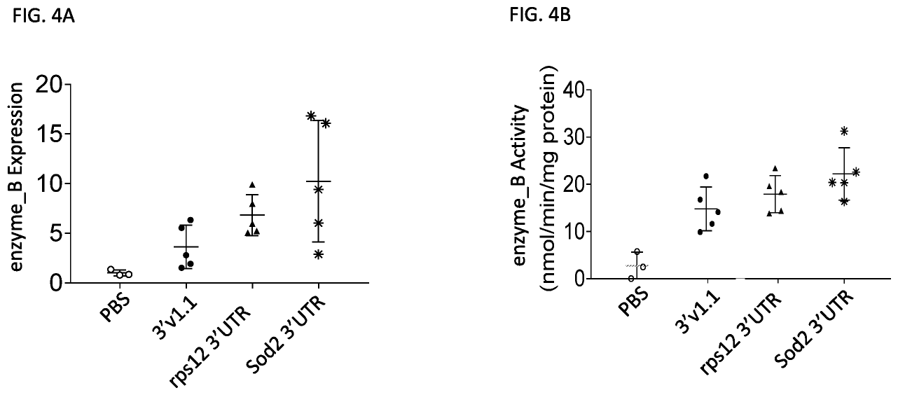

The mRNAs evaluated as shown in FIGS. 4A-4B comprised 3′UTRs that were described in Example 3. These included a rps12 3′UTR and a Sod2 3′UTR. These were compared to a reference 3′UTR (3′v1.1).

The mRNAs were evaluated in mice deficient in enzyme_B. Animals were injected on day 0 and on day 14 intravenously with phosphate-buffered saline (PBS) or with 0.3 mg/kg of mRNA formulated in a lipid nanoparticle comprising 50:38.5:10:1.5 ionizable lipid 1:DSPC:cholesterol:PEG lipid 1. Enzyme_B expression and enzymatic activity was determined in lysates generated from mouse livers that were harvested on day 15. Expression was determined by capillary electrophoresis and activity was measured by high performance liquid chromatography (HPLC). Background expression and activity was determined in mice injected with PBS only. Additionally, two different biomarkers of enzyme_B activity (enzyme_B-BM1 and enzyme_B-BM2) were quantified in plasma. Both biomarkers are expected to accumulate in enzyme_B deficient mice. Biomarker levels were quantified by liquid chromatography-tandem mass spectrometry (LC-MS/MS).

As shown in FIG. 4A, treatment with mRNA encoding enzyme_B gave increased expression relative to background for all 3′UTRs evaluated. However, treatment with a rps12 3′UTR or a Sod2 3′UTR gave increased expression relative to the reference 3′UTR (3′v1.1). Similarly, as shown in FIG. 4B, a rps12 3′UTR or a Sod2 3′UTR also yielded increased enzyme_B activity compared to a reference 3′UTR (3′v1.1).

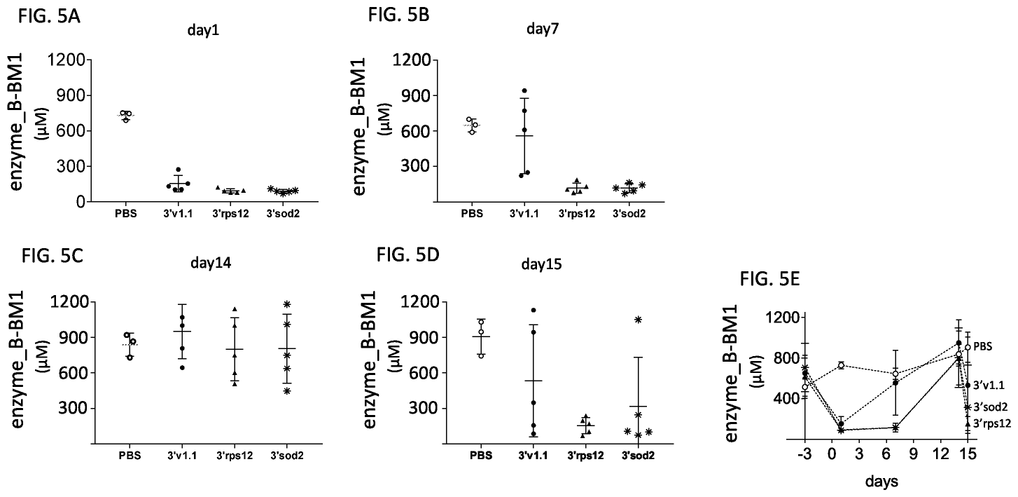

Biomarker levels of enzyme_B-BM1 were evaluated over time (n=5 mice). Animals injected with PBS only had high levels throughout the study (FIG. 5A-5E), as expected for enzyme_B-deficiency. Treatment with mRNA having a reference 3′UTR (3′v1.1) gave reduced levels at 24 hours post-injection (FIG. 5A). However, the levels increased by 7 days post-injection (FIG. 5B). In contrast, treatment with mRNA having a rps12 3′UTR or a Sod2 3′UTR also gave decreased levels that persisted through at least day 7 (FIG. 5B). Following the second dose of mRNA, the mRNA having a rps12 3′UTR or a Sod2 3′UTR gave lower biomarker levels than mRNA having a reference 3′UTR (3′v1.1) (FIGS. 5C-5D). FIG. 5E shows the average biomarker level over time.

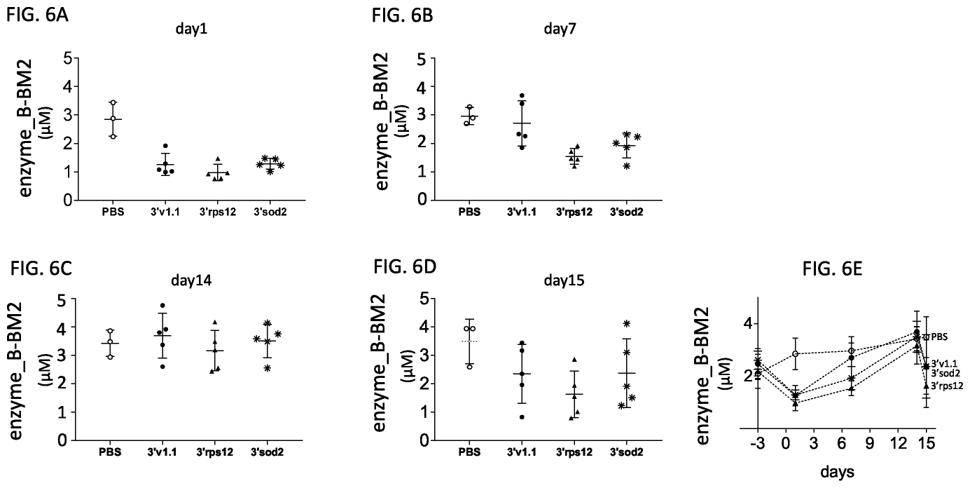

A second biomarker of enzyme_B activity (enzyme_B-BM2) exhibited a similar pattern, as shown in FIGS. 6A-6E. Low biomarker levels persisted through at least day 7 for mRNA with a rps12 3′UTR or a Sod2 3′UTR, but not with a reference 3′UTR (FIG. 6B). Thus, inclusion of a rps12 3′UTR or a Sod2 3′UTR prolongs enzyme activity following a single administration of mRNA.

### Example 5: Combinations of 5′ and 3′ UTRs with Improved In Vivo Expression of mRNA Encoding an Intracellular or Secreted Protein

As described in Example 1-2, an RNAse P stem loop in the 5′UTR provides increased mRNA expression and activity of a translated protein. Additionally, 3′UTRs described in Examples 3-4 result in similar improvements. The effect of combining the described 5′ and 3′ UTRs on mRNA expression was thus evaluated on expression of an encoded intracellular protein (luciferase) or a secreted protein (erythropoietin) in vivo. The combinations of 5′UTR and 3′UTRs that were evaluated are shown in Table 19. The mRNAs were prepared by in vitro-transcription and fully modified with N1-methyl pseudouridine (m¹ψ) in place of uracil.

TABLE 19
mRNAs encoding an intracellular or secreted protein and
combining exemplary 5′UTR and 3′UTRs

| Identifier | Name | SEQ ID NO |
|---|---|---|
| 5′UTR | 5′v1.1 | 4 |
| 5′UTR | RNAseP_p2 | 120 |
| 3′UTR | 3′v1.1 | 70 |
| 3′UTR | rps12 3′UTR | 76 |
| 3′UTR | Sod2 3′UTR | 78 |
| 3′UTR | Oxa1 3′UTR | 74 |
| mRNA Identifier | 5′UTR | 3′UTR |
| 5′v1.1_3′v1.1 | 5′v1.1 | 3′v1.1 |
| 5′v1.1_3′rps12d | 5′v1.1 | Rps12 3′UTR |
| 5′v1.1_3′sod2 | 5′v1.1 | Sod2 3′UTR |
| 5′v1.1_3′oxa1sh | 5′v1.1 | Oxa1 3′UTR |
| 5′p2_3′rps12d | RNAseP_p2 | Rps12 3′UTR |
| 5′p2_3′sod2 | RNAseP_p2 | Sod2 3′UTR |
| 5′p2_3′oxa1sh | RNAseP_p2 | Oxa1 3′UTR |

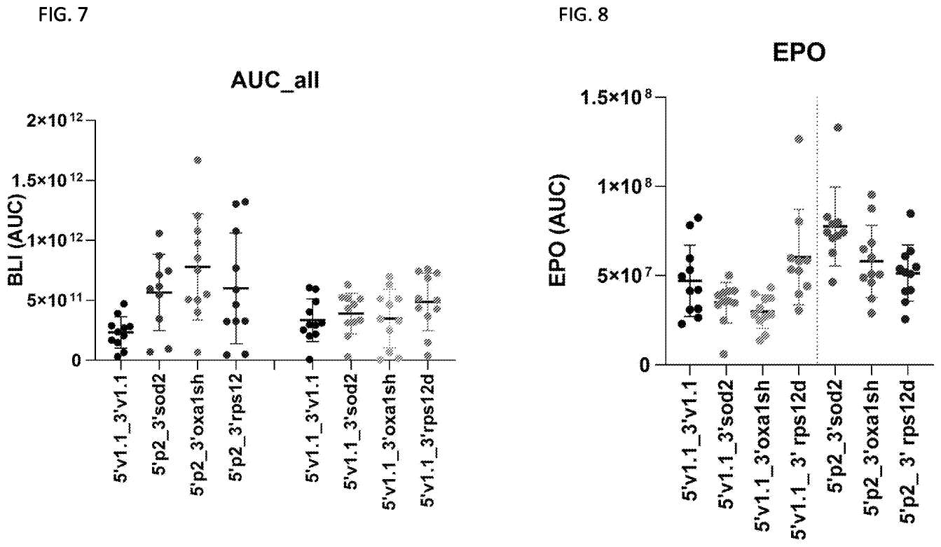

The mRNA constructs as described above and encoding luciferase were evaluated in vivo. Briefly, wild-type mice were injected on day 0 intravenously with 0.5 mg/kg of mRNAs formulated in lipid nanoparticles comprising 50:38.5:10:1.5 ionizable lipid 1:DSPC:cholesterol:PEG lipid 1. In vivo luciferase expression was measured in sedated mice at various time points (2, 6, 24, 30 and 48 hours). Luciferin, the substrate of luciferase, was injected intraperitoneally into mice at a dose of 150 mg/kg body weight. At 20 minutes after Luciferin injection, animals were euthanized. Whole body imaging was carried out and bioluminescent signal intensity was analyzed on the IVIS spectrum by using Living Image Software (Perkin Elmer, Waltham, Mass.) and expressed as photons/sec/cm2/sr. Area under the curve was calculated using Prism software (GraphPad Software, Inc., La Jolla, Calif.). As shown in FIG. 7, bioluminescence levels were slightly elevated above control for an mRNA with a reference 5′UTR and any of the 3′UTRs evaluated, including the Sod2 3′UTR, the Oxal 3′UTR, or the Rps12 3′UTR. However, bioluminescence levels were most increased for mRNAs comprising a combination of a 5′UTR with an RNAse P stem loop and any of the 3′UTRs evaluated.

The mRNA constructs as described above and encoding erythropoietin were evaluated in vivo. Briefly, wild-type mice were injected on day 0 intravenously with 0.5 mg/kg of mRNAs formulated in lipid nanoparticles comprising 50:38.5:10:1.5 compound X:DSPC:cholesterol:PEG/DMG. Erythropoietin (Epo) expression in blood was measured at 2, 6, 24, 30 and 48 h. Briefly, serum was prepared from blood samples and analyzed for erythropoietin levels using human EPO ELISA kits (StemCell Technologies #01630) following the manufacturer's instructions. Area under the curve was calculated using Prism software (GraphPad Software, Inc., La Jolla, Calif.). As shown in FIG. 8, Epo expression levels were slightly improved relative to control mRNA for mRNAs comprising a reference 5′UTR and a Rps12 3′UTR. However, the combination of a 5′UTR with an RNAse P stem loop and either a Sod2 3′UTR or an Oxal 3′UTR resulted in the greatest improvement in mRNA expression relative to control mRNA.

### Example 6: Combinations of 5′ and 3′ UTRs Decrease Enzyme Deficiency-Related Phenotypes in a Murine Model

The effect of combining a 5′UTR with an RNAse P stem loop and a Sod2 3′UTR on expression and activity of mRNA encoding a cellular enzyme was determined in vivo. The mRNAs encoded enzyme_A and were prepared by in vitro-transcription. The mRNA was fully modified with N1-methyl pseudouridine (m¹ψ) in place of uracil. The combination of 5′UTR and 3′UTRs evaluated are shown in Table 20. The 3′UTR designated “3′v1.1_miR-142-3p” contains a miR-142-3p binding site that is shown by SEQ ID NO: 179. A control mRNA encoding eGFP was prepared with a 5′v1.1 and 3′v1.1 UTRs. The mRNA “5′v1.1, 3′v1.1m142” and the mRNA “5′P2, 3′v1.1” comprised an ORF encoding a first N-terminal mitochondrial targeting sequence 1 (referred to as MTS1). The mRNA “5′P2, 3′sod2” comprised an ORF encoding a second N-terminal MTS (referred to as MTS2).

TABLE 20
mRNAs encoding enzyme_A that combine exemplary 5′UTR and 3′UTRs

| Identifier | Name | SEQ ID NO |
|---|---|---|
| 5′UTR | 5′v1.1 | 4 |
| 5′UTR | RNAseP_p2 | 120 |
| 3′UTR | 3′v1.1 | 70 |
| 3′UTR | 3′v1.1_miR-142-3p | 163 |
| 3′UTR | Sod2 3′UTR | 78 |
| mRNA Identifier | 5′UTR | 3′UTR |
| eGFP | 5′v1.1 | 3′v1.1 |
| 5′v1.1, 3′v1.1m142 | 5′v1.1 | 3′v1.1_miR-142-3p |
| 5′P2, 3′v1.1 | RNAseP_p2 | 3′v1.1 |
| 5′P2, 3′sod2 | RNAseP_p2 | Sod2 3′UTR |

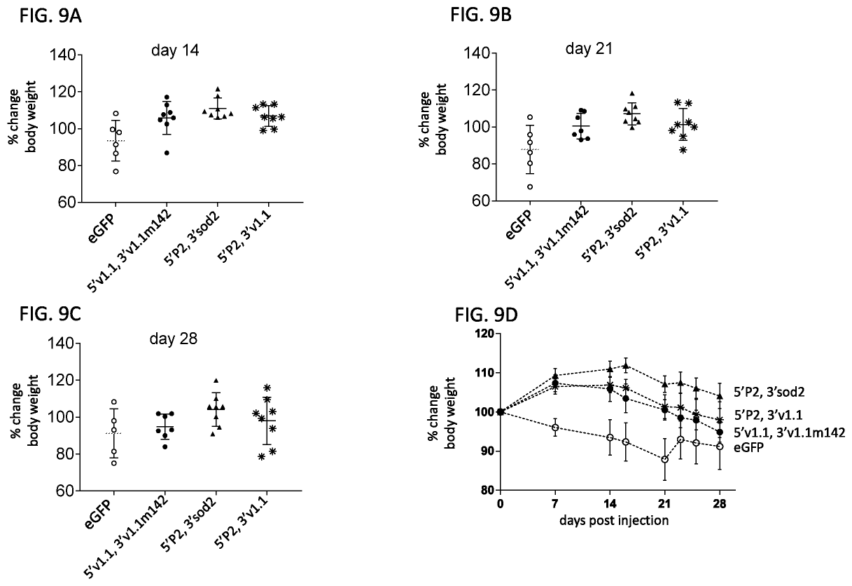

The mRNA constructs were evaluated in vivo. Briefly, 0.5 mg/kg of mRNA formulated in a lipid nanoparticle comprising 50:38.5:10:1.5 ionizable lipid 1:DSPC:cholesterol:PEG lipid 1 was administered to enzyme_A-deficient mice by intravenous injection on day 0 and 31. Animals deficient in enzyme_A exhibit weight loss that is prevented with recovery of enzyme_A expression. Thus, animals were evaluated for change in body weight relative to day 0. Shown is change in body weight on day 14 (FIG. 9A), day 21 (FIG. 9B) and day 28 (FIG. 9C) and over time (FIG. 9D). Control animals treated with eGFP-encoding mRNA had reduced body weight by day 14 that continued through the course of the study. Additionally, 3/8 starting animals died during the course of the study in the eGFP-treated group. Fewer or no animals died in the groups treated with 5′v1.1, 3′v1.1m142 (1/8), 5′P2, 3′sod2 (0/8), or 5′P2, 3′v1.1 (0/8) mRNA encoding enzyme_A. Animals treated with mRNAs having a RNAse P stem loop 5′UTR and a reference 3′UTR showed less weight loss than control animals. While treatment with mRNA having both an RNAse P stem loop 5′UTR and a Sod2 3′UTR demonstrated no significant change in body weight throughout the study (FIGS. 9A-9D).

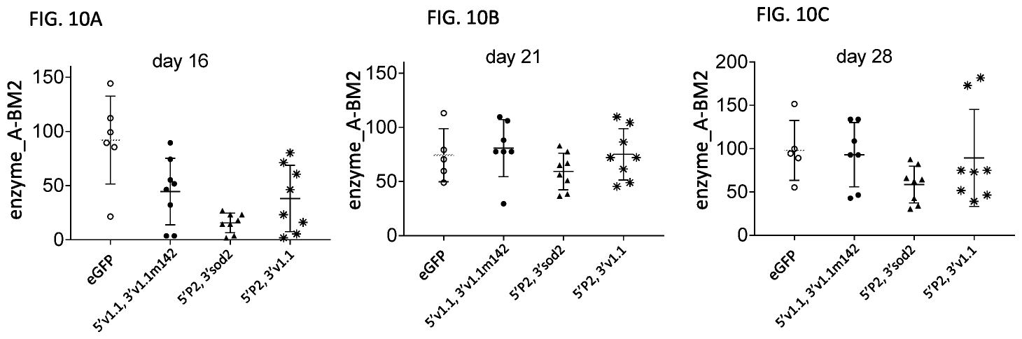

As an additional indicator of efficacy, plasma levels of a biomarker of enzyme_A (e.g., enzyme_A-BM2) was measured on days 16, 21 and 28 using a commercially available quantitative colorimetric assay (FIGS. 10A-10C). The biomarker accumulates in animals deficient in enzyme_A, thus control animals exhibited high biomarker levels throughout the course of the study. Treatment with mRNA encoding enzyme_A resulted in decreased biomarker levels by day 16 (FIG. 10A). While treatment with mRNA having both a RNAse P stem loop 5′UTR and a Sod2 3′UTR demonstrated the most substantial reduction in biomarker levels on each day tested (FIG. 10A-C).

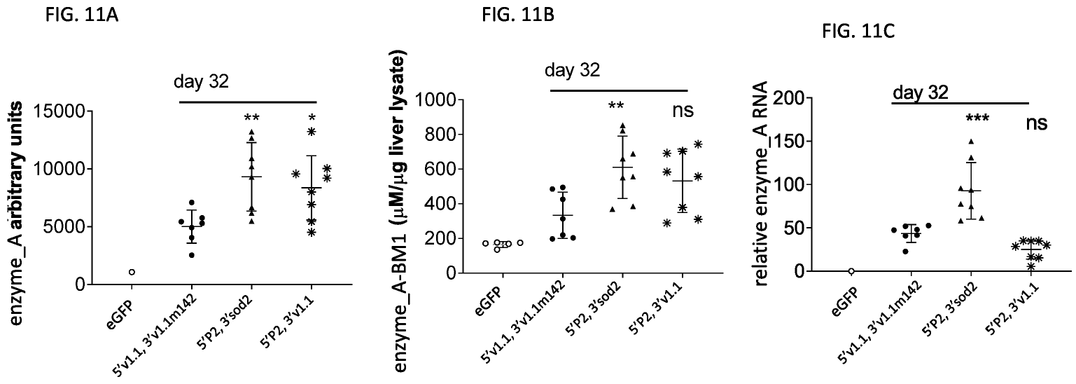

The levels of enzyme_A protein expression (FIG. 11A), enzymatic activity (FIG. 11B), and mRNA quantity (FIG. 11C) were measured in liver lysates harvested on day 32. The abundance of enzyme_A protein was measured by capillary electrophoresis. Activity was determined by measuring a protein product of the enzyme_A enzymatic reaction (referred to as a first biomarker of enzyme_A or enzyme_A-BM1) by an in house colorimetric assay. The level of enzyme_A mRNA present in liver lysate was measured by a branched DNA assay and shown in FIG. 11C as a relative abundance to control animals treated with eGFP mRNA.

Treatment with mRNA encoding enzyme_A resulted in increased levels of enzyme_A mRNA, enzyme_A protein, and enzymatic activity compared to control animals treated with eGFP mRNA. The level of enzyme_A protein present in liver lysates was significantly higher for animals treated with mRNA having both an RNAse P stem loop 5′ UTR and a Sod2 3′UTR (FIG. 11A) than one with reference UTRs (5′v1.1, 3′v1.1m142). Additionally, treatment with mRNA having both an RNAse P stem loop 5′UTR and a Sod2 3′UTR resulted in significantly higher levels of enzyme_A enzymatic activity (FIG. 11B) and enzyme_A mRNA (FIG. 11C) in liver lysates compared to mRNA comprising reference UTRs (5′v1.1, 3′v1.1m142 or 5′P2, 3′v1.1).

Thus, the combination of an RNAse P stem loop 5′ UTR and a Sod2 3′UTR is beneficial for maintaining animal body weight (FIGS. 9A-9D), reducing plasma levels of enzyme_A-BM2 (FIGS. 10A-10C), and increasing tissue levels of enzyme_A mRNA, protein and activity (FIGS. 11A-11C).

### Example 7 Combinations of 5′ and 3′ UTRs Enhance In Vivo Expression and Activity of a Cellular Enzyme Encoded by an mRNA

Combinations of 5′ and 3′UTRs that were identified for improved mRNA potency were evaluated for their effect on mRNA expression and activity of translated protein in vivo. The mRNAs encoded enzyme_B and comprised combinations of 5′UTRs and 3′UTRs as shown in Table 21. The 3′UTRs shown by SEQ ID NO: 170 and SEQ ID NO: 172 were derived from a Sod2 3′UTR and comprise three miR-142-3p binding sites (SEQ ID NO: 179) in different positions of the UTR, either near the 3′edge of the UTR (Sod2_3, SEQ ID NO: 170) or near the 5′edge of the UTR (Sod2_5, SEQ ID NO: 172). Similar designations are used for 3′UTRs derived from a rps12 3′UTR. Additionally, they included a triple stop codon (SEQ ID NO: 183) to promote termination of translation and prevent ribosomal readthrough of the stop codon. The mRNAs were prepared by in vitro-transcription and fully modified with N1-methyl pseudouridine (m¹ψ) in place of uracil.

A control mRNA encoding enzyme_B comprised a reference 5′v1.0 and 3′v1.1_miR-142-3p UTR with a single miR142 site. The control mRNA was altered to minimize uracil content and fully modified to contain 5-methoxy uridine (mo⁵U) in place of remaining uracil.

TABLE 21
mRNA encoding enzyme_B that combine exemplary 5′UTR and 3′UTRs

| Identifier | Name | SEQ ID NO |
|---|---|---|
| 5′UTR | 5′v1.0 | 45 |
| 5′UTR | RNAseP_p2 | 120 |
| 5′UTR | F153 | 41 |
| 3′UTR | 3′v1.1_miR-142-3p | 163 |
| 3′UTR | Sod2_3 | 170 |
| 3′UTR | Sod2_5 | 172 |
| 3′UTR | rpS12_3 | 174 |
| 3′UTR | rpS12_5 | 176 |
| mRNA Identifier | 5′UTR | 3′UTR |
| 5′v1_3′v1.1 | 5′v1.0 | 3′v1.1_miR-142-3p |
| P2_Sod2_3 | RNAseP_p2 | Sod2_3 |
| P2_Sod2_5 | RNAseP_p2 | Sod2_5 |
| P2_rps12_3 | RNAseP_p2 | rps12_3 |
| P2_rps12_5 | RNAseP_p2 | rps12_5 |
| F153_Sod2_3 | F153 | Sod2_3 |
| F153_Sod2_5 | F153 | Sod2_5 |
| F153_rps12_3 | F153 | rps12_3 |
| F153_rps12_5 | F153 | rps12_5 |

Wild-type mice were injected on day 0 intravenously with phosphate-buffered saline (PBS) or with 0.5 mg/kg of the mRNAs encoding enzyme_B formulated in a lipid nanoparticle. 24 hours post-injection, the expression level and enzymatic activity of enzyme_B protein was determined in lysates generated from mouse livers. The abundance of enzyme_B protein in liver lysates was measured by capillary electrophoresis. The enzymatic activity of enzyme_B protein was measured according the methods described in Example 4. Liver lysates generated from control animals injected with PBS were used to determine a background level of enzyme_B protein abundance and enzymatic activity.

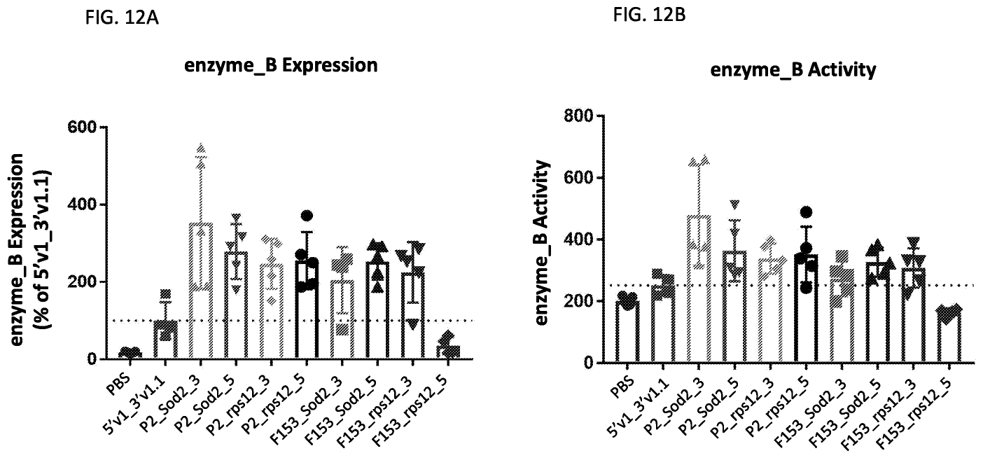

Enzyme expression and activity were modestly improved for control mRNA encoding enzyme_B (5′vl_3′v1.1) compared to background as shown in FIGS. 12A-12B. The mRNAs F153_Sod2_3, F153_Sod2_5, and F153_rps12_3 with a F153 and either a Sod2 3′UTR or a rps12 3′UTR had higher expression and activity levels than control mRNA (5′vl_3′v1.1). Similarly, the mRNAs P2_rps12_3 and P2_rps12_5 with a RNAse P stem loop 5′UTR and a rps12 3′UTR had increased expression and activity compared to control mRNA (5′vl_3′v1.1). Significantly, treatment with mRNAs P2_Sod2_3 and P2_Sod2_5 having an RNAse P stem loop 5′UTR and a Sod2 3′UTR demonstrated the highest increase in enzyme expression and activity.

### Example 8: Combinations of 5′ and 3′ UTRs and Lipid Nanoparticle Formulations Enhance in Vivo mRNA Expression and Activity of an Encoded Cellular Enzyme

The effect of lipid nanoparticle formulation on mRNA expression and activity of translated protein was determined in vivo. mRNA encoding enzyme_B were delivered using different lipid nanoparticle formulations, with each lipid nanoparticle comprising an ionizable lipid, a sterol, a phospholipid, and a polyethylene glycol (PEG) lipid. The structural components and ratios used to generate the LNPs are shown in Table 22. The mRNAs evaluated corresponded to those identified in Example 7.

TABLE 22
structural components for lipid nanoparticles

| | Ionizable | Non-cationic helper | Structural | PEG lipid | |
|---|---|---|---|---|---|
| LNP | lipid (I) | lipid/Phospholipid (H) | lipid (S) | (P) | Ratio (I:S:H:P) |
| LNP-1 | I1 | DSPC | cholesterol | P1 | 50:38.5:10:1.5 |
| LNP-2 | I1 | DSPC | Cholesterol | P2 | 50:38:10:2 |
| LNP-3 | I2 | DSPC | Cholesterol | P2 | 50:38:10:2 |

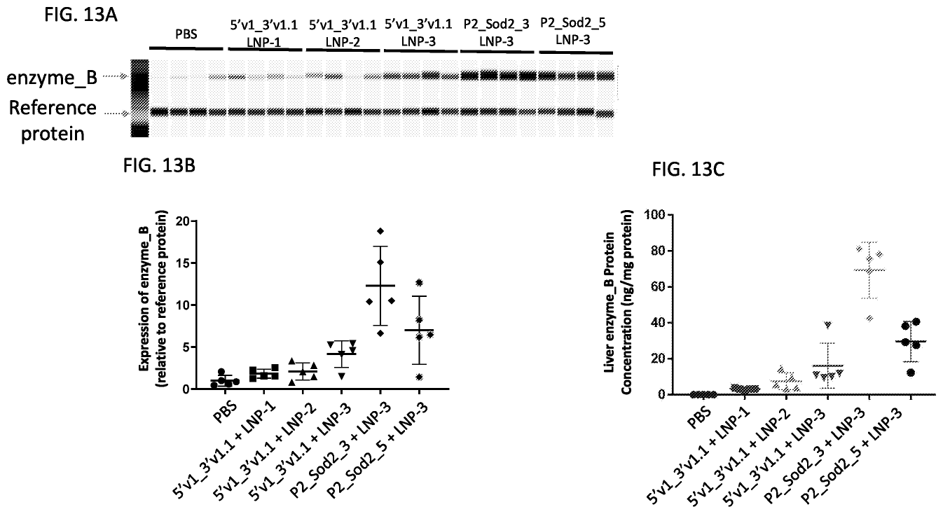

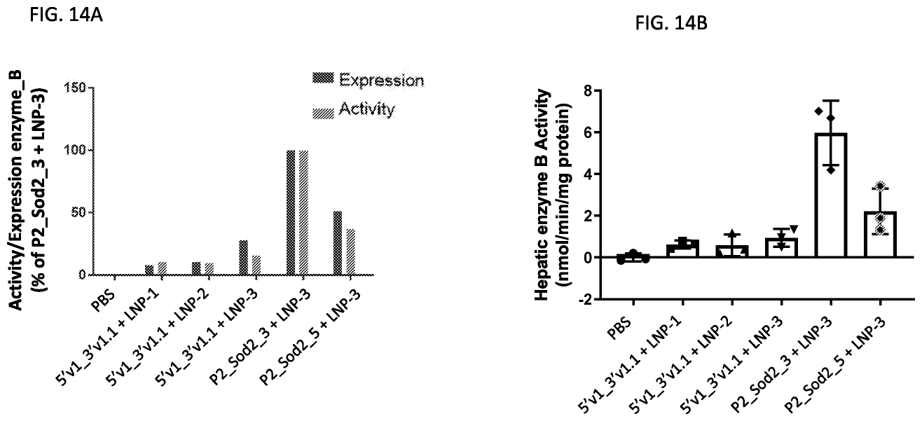

The mRNA were formulated in LNPs shown in Table 22 and administered to enzyme_B-deficient mice by intravenous tail vein injection at a dose of 0.2 mg/kg of mRNA. Control animals were administered PBS by intravenous injection. The animals were sacrificed and organs were harvested at 24 hours post administration. Enzyme_B expression level in liver lysates was evaluated by assessing protein quantity by quantitative capillary electrophoresis, with an exemplary immunoblot generated by capillary electrophoresis shown in FIG. 13A. The immunoblot was stained for detection of reference protein to ensure similar loading of cell lysate between samples. Quantification of enzyme_B band intensity and normalization of the average band intensity for each group to the average band intensity of the PBS control group provided relative levels of enzyme_B expression between treatment groups as shown in FIG. 13B. As an alternative method for measuring expression level, quantity of enzyme_B in liver lysates was also measured by LC-MS (FIG. 13C). The level of enzyme_B enzymatic activity in liver lysates was also measured as described in Example 4 (FIGS. 14A-14B).

Expression was elevated over background with treatment of mRNA having reference 5′v1.1 and 3′v1.1 UTRs. The reference mRNA had higher expression if it was formulated in LNP-3 compared to formulation in an LNP-1 or LNP-2 preparation (FIGS. 13A-13C, FIG. 14). Additionally, the reference mRNA had higher activity if it was formulated in an LNP-3 compared to formulation in a LNP-1 or LNP-2 (FIG. 14). The greatest increase in mRNA expression was seen for P2_Sod2_3 mRNA formulated with an LNP-3 preparation (FIGS. 13A-13C, FIGS. 14A-14B). Additionally, treatment with P2_Sod2_3 mRNA gave the highest activity levels (FIGS. 14A-14B). Thus, mRNA comprising an RNAse P stem loop in the 5′UTR and a Sod2 3′UTR with miR-142-3p binding sites near the 3′termini (SEQ ID NO: 170) delivered in an LNP-3 formulation resulted in high enzyme expression and activity in vivo.

### Example 9: Combinations of 5′ and 3′ UTRs and Lipid Nanoparticle Formulations that Increase Potency of mRNA Encoding a Cellular Enzyme In Vivo

Additional markers of potency were evaluated for mRNAs comprising a 5′UTR with an RNAse P stem loop and a Sod2 3′UTR and formulated with an LNP-3 preparation. The mRNAs and LNP formulations were administered to enzyme_B deficient mice as described in Example 8 and a biomarker of enzyme_B expression (enzyme_B-BM1) was measured in plasma and in tissue lysates of the liver, kidney and heart according to the methods described in Example 4. Enzyme_B-BM1 accumulates in tissues of animals deficient in enzyme_B and is expected to decrease with increased enzyme_B expression and activity.

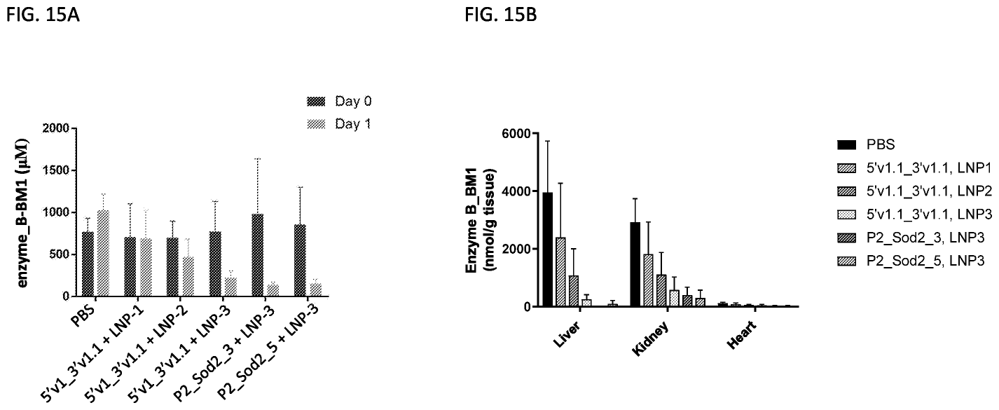

Biomarker levels in plasma were measured prior to treatment (e.g., day 0) and at 24 hours following treatment (e.g., day 1) as shown in FIG. 15A. The levels were similar for all treatment groups prior to administration of mRNA. At 24 hours, levels remained high for control animals treated with PBS, while levels were reduced for animals treated with enzyme_B-encoding mRNA. In particular, treatment with reference mRNA (5′v1.1_3′v1.1) in a LNP-3 formulation gave lower levels than an LNP-1 or LNP-2 formulation. The most dramatic reduction in biomarker levels was seen for P2_Sod2_3 and P2_Sod2_5 mRNAs comprising a 5′UTR with an RNAse P stem loop and Sod2 3′UTRs (FIG. 15A). The change in plasma biomarker levels following treatment relative to before treatment are shown in Table 23.

Biomarker levels in liver, kidney and heart lysates harvested at 24 hours following treatment showed a similar trend (FIG. 15B). Treatment with mRNA P2_Sod2_3 or P2_Sod2_5 formulated with LNP-3 resulted in the most substantial reductions in biomarker levels in tissue lysates. Together, these results indicate that delivery of mRNA comprising a 5′UTR with an RNAse P stem loop and a Sod2 3′UTR with an LNP-3 formulation results in the most potent reduction of an enzyme_B biomarker.

TABLE 23
potency of enzyme_B-encoding mRNA as measured by enzyme_B
expression, activity, and reduction in biomarker levels

| | | | 5′v1.1- | 5′v1.1- | 5′v1.1- | P2_Sod2_ | P2_Sod2_ |
|---|---|---|---|---|---|---|---|
| | | | _3′v1.1 + | _3′v1.1 + | _3′v1.1 + | 3 + | 5 + |
| Potency parameter | | PBS | LNP-1 | LNP-2 | LNP-3 | LNP-3 | LNP-3 |
| enzyme_B | | 0 | 3.182 | 7.5 | 16.162 | 69.294 | 29.618 |
| expression | | | | | | | |
| by LC-MS | | | | | | | |
| (ng enzyme_ | | | | | | | |
| B/mg total protein) | | | | | | | |
| enzyme_B | | 1.00 | 1.90 | 2.19 | 4.14 | 12.34 | 6.80 |
| expression | | | | | | | |
| (CE quantification | | | | | | | |
| relative | | | | | | | |
| to another protein | | | | | | | |
| used as standard) | | | | | | | |
| enzyme_B acitivity | | 1.00 | 1.13 | 1.12 | 1.20 | 2.28 | 1.48 |
| (Fold change | | | | | | | |
| to PBS) | | | | | | | |
| % change in plasma | | 33.6 | −2.0 | −33.1 | −71.5 | −86.0 | −82.4 |
| enzyme_B-BM1 | | | | | | | |
| (day 1 vs day0) | | | | | | | |
| % change | Liver | — | −39.2 | −72.7 | −93.7 | −99.8 | −97.5 |
| in tissue | Kidney | — | −50.2 | −62.0 | −80.3 | −86.3 | −89.7 |
| enzyme_B- | Heart | — | −28.1 | −54.1 | −61.7 | −84.2 | −81.0 |
| BMI from | | | | | | | |
| PBS | | | | | | | |

SEQUENCE LISTING

| SEQ | | | |
|---|---|---|---|
| ID | | | |
| NO | Identifier | | Sequence |
| 1 | RNA Element | V1 | CCCCGGCGCC |
| 2 | RNA Element | V2 | CCCCGGC |
| 3 | RNA Element | EK1 | CCCGCC |
| 4 | 5′UTR (RNA) | 5′v1.1 | GGGAAAUAAGAGAGAAAAGAAGAGUAAGAAGAAA |
| | | (V1) | UAUAAGACCCCGGCGCCGCCACC |
| 5 | RNA Element | RNAse P stem | TCTCCCTGAGCTTCAGGGAG |
| | (DNA) | loop | |
| 6 | RNA Element | RNAse P stem | UCUCCCUGAGCUUCAGGGAG |
| | (RNA) | loop | |
| 7 | RNA Element | MRP stem loop | AGAAGCGTATCCCGCTGAGC |
| | (DNA) | | |
| 8 | 5′UTR (RNA) | 5′v1.0 | UAAGAGAGAAAAGAAGAGUAAGAAGAAAUAUAAG |
| | | Core (minus | A |
| | | leader, Kozak) | |
| 9 | 5′UTR (DNA) | 5′v1.1 | GGGAAATAAGAGAGAAAAGAAGAGTAAGAAGAAA |
| | | (Vl) | TATAAGACCCCGGCGCCGCCACC |
| 10 | 5′UTR (DNA) | V2-5′UTR | GGGAAATAAGAGAGAAAAGAAGAGTAAGAAGAAA |
| | | (V2) | TATAAGACCCCGGCGCCACC |
| 11 | 5′UTR (DNA) | CG1-5′UTR | GGGAAATAAGAGAGAAAAGAAGAGTAAGAAGAAA |
| | | | TATAAGAGCGCCCCGCGGCGCCCCGCGGCCACC |
| 12 | 5′UTR (DNA) | CG2-5′UTR | GGGAAATAAGAGAGAAAAGAAGAGTAAGAAGAAA |
| | | | TATAAGACCCGCCCGCCCCGCCCCGCCGCCACC |
| 13 | 5′UTR | KT1-UTR | GGGCCCGCCGCCAAC |
| 14 | 5′UTR | KT2-UTR | GGGCCCGCCGCCACC |
| 15 | 5′UTR | KT3-UTR | GGGCCCGCCGCCGAC |
| 16 | 5′UTR | KT4-UTR | GGGCCCGCCGCCGCC |
| 17 | RNA Element | Kozak-1 | GCCACC |
| 18 | RNA Element | EK2 | GCCGCC |
| 19 | RNA Element | EK3 | CCGCCG |
| 20 | RNA Element | CG1 | GCGCCCCGCGGCGCCCCGCG |
| 21 | RNA Element | CG2 | CCCGCCCGCCCCGCCCCGCC |
| 22 | RNA Element | (CCG)ₙ, | [CCG]ₙ |
| | | n = 1-10 | |
| 23 | RNA Element | (GCC)ₙ, | [GCC]ₙ |
| | | n = 1-10 | |
| 24 | RNA Element | SL1 | CCGCGGCGCCCCGCGG |
| 25 | RNA Element | SL2 | GCGCGCAUAUAGCGCGC |
| 26 | RNA Element | SL3 | CATGGTGGCGGCCCGCCGCCACCATG |
| | (DNA) | | |
| 27 | RNA Element | SL4 | CATGGTGGCCCGCCGCCACCATG |
| | (DNA) | | |
| 28 | RNA Element | SL5 | CATGGTGCCCGCCGCCACCATG |
| | (DNA) | | |
| 29 | RNA Element | CR2 | CCCCCCCAACCC |
| 30 | RNA Element | CR1 | CCCCCCCCAACC |
| 31 | RNA Element | CR3 | CCCCCCACCCCC |
| 32 | RNA Element | CR4 | CCCCCCUAAGCC |
| 33 | RNA Element | CR5 | CCCCACAACC |
| 34 | RNA Element | CR6 | CCCCCACAACC |
| 35 | 5′UTR (RNA) | combo1_V1.1 | GGGAAACCCCCCACCCCCGGGGAAAUAAGAGAGA |
| | | (CR3, V1) | AAAGAAGAGUAAGAAGAAAUAUAAGACCCCGGCG |
| | | | CCGCCACC |
| 36 | 5′UTR (RNA) | combo2_V1.1 | GGGAAAUCCCCACAACCGGGGAAAUAAGAGAGAA |
| | | (CR5, V1) | AAGAAGAGUAAGAAGAAAUAUAAGACCCCGGCGC |
| | | | CGCCACC |
| 37 | 5′UTR (RNA) | combol_S065 | GGGAAACCCCCCACCCCCGCCUCAUAUCCAGGCU |
| | | (CR3) | CAAGAAUAGAGCUCAGUGUUUUGUUGUUUAAUCA |
| | | | UUCCGACGUGUUUUGCGAUAUUCGCGCAAAGCAG |
| | | | CCAGUCGCGCGCUUGCUUUUAAGUAGAGUUGUUU |
| | | | UUCCACCCGUUUGCCAGGCAUCUUUAAUUUAACA |
| | | | UAUUUUUAUUUUUCAGGCUAACCUAAAGCAGAGA |
| | | | A |
| 38 | 5′UTR (RNA) | combo2_S065 | GGGAAAUCCCCACAACCGCCUCAUAUCCAGGCUC |
| | | (CR5) | AAGAAUAGAGCUCAGUGUUUUGUUGUUUAAUCAU |
| | | | UCCGACGUGUUUUGCGAUAUUCGCGCAAAGCAGC |
| | | | CAGUCGCGCGCUUGCUUUUAAGUAGAGUUGUUUU |
| | | | UCCACCCGUUUGCCAGGCAUCUUUAAUUUAACAU |
| | | | AUUUUUAUUUUUCAGGCUAACCUAAAGCAGAGAA |
| 39 | 5′UTR (RNA) | combo3_S065 | GGGAGACCUCAUAUCCAGGCUCAAGAAUAGAGCU |
| | | (S065 core | CAGUGUUUUGUUGUUUAAUCAUUCCGACGUGUUU |
| | | extended Kozak) | UGCGAUAUUCGCGCAAAGCAGCCAGUCGCGCGCU |
| | | | UGCUUUUAAGUAGAGUUGUUUUUCCACCCGUUUG |
| | | | CCAGGCAUCUUUAAUUUAACAUAUUUUUAUUUUU |
| | | | CAGGCUAACCUACGCCGCCACC |
| 40 | 5′UTR (RNA) | combo4_S065 | GGGAAACCCCCCACCCCCGCCUCAUAUCCAGGCU |
| | | (CR3) | CAAGAAUAGAGCUCAGUGUUUUGUUGUUUAAUCA |
| | | | UUCCGACGUGUUUUGCGAUAUUCGCGCAAAGCAG |
| | | | CCAGUCGCGCGCUUGCUUUUAAGUAGAGUUGUUU |
| | | | UUCCACCCGUUUGCCAGGCAUCUUUAAUUUAACA |
| | | | UAUUUUUAUUUUUCAGGCUAACCUACGCCGCCAC |
| | | | C |
| 41 | 5′UTR (RNA) | F153, | GGGAAAUCCCCACAACCGCCUCAUAUCCAGGCUC |
| | | combo5_S065 | AAGAAUAGAGCUCAGUGUUUUGUUGUUUAAUCAU |
| | | (CR5) | UCCGACGUGUUUUGCGAUAUUCGCGCAAAGCAGC |
| | | | CAGUCGCGCGCUUGCUUUUAAGUAGAGUUGUUUU |
| | | | UCCACCCGUUUGCCAGGCAUCUUUAAUUUAACAU |
| | | | AUUUUUAUUUUUCAGGCUAACCUACGCCGCCACC |
| 42 | 5′UTR (RNA) | 3065 | GGGAGACCUCAUAUCCAGGCUCAAGAAUAGAGCU |
| | | | CAGUGUUUUGUUGUUUAAUCAUUCCGACGUGUUU |
| | | | UGCGAUAUUCGCGCAAAGCAGCCAGUCGCGCGCU |
| | | | UGCUUUUAAGUAGAGUUGUUUUUCCACCCGUUUG |
| | | | CCAGGCAUCUUUAAUUUAACAUAUUUUUAUUUUU |
| | | | CAGGCUAACCUAAAGCAGAGAA |
| 43 | 5 ′UTR | GCC3-ExtKozak | GGGAAAGCCGCCGCCGCCACC |
| 44 | 5′UTR (RNA) | CrichCR4 + GCC3- | GGGAAACCCCCCUAAGCCGCCGCCGCCGCCACC |
| | | ExtKozak | |
| | | (CR4, GCC₃) | |
| 45 | 5′UTR (RNA) | 5′v1.0 | GGGAAAUAAGAGAGAAAAGAAGAGUAAGAAGAAA |
| | | | UAUAAGAGCCACC |
| 46 | 5′UTR (RNA) | S065 core | CCUCAUAUCCAGGCUCAAGAAUAGAGCUCAGUGU |
| | | | UUUGUUGUUUAAUCAUUCCGACGUGUUUUGCGAU |
| | | | AUUCGCGCAAAGCAGCCAGUCGCGCGCUUGCUUU |
| | | | UAAGUAGAGUUGUUUUUCCACCCGUUUGCCAGGC |
| | | | AUCUUUAAUUUAACAUAUUUUUAUUUUUCAGGCU |
| | | | AACCUA |
| 47 | RNA Element | MRP stem loop | AGAAGCGUAUCCCGCUGAGC |
| | (RNA) | (RNA) | |
| 48 | RNA Element | Kozak-2 | GCCGCC |
| 49 | RNA Element | SL3 | CAUGGUGGCGGCCCGCCGCCACCAUG |
| | (RNA) | | |
| 50 | RNA Element | SL4 | CAUGGUGGCCCGCCGCCACCAUG |
| | (RNA) | | |
| 51 | RNA Element | SL5 | CAUGGUGCCCGCCGCCACCAUG |
| | (RNA) | | |
| 52 | RNA Element | F153 GC | CGCC |
| 53 | 5′UTR leader | G-start | GGGAAA |
| 54 | 5′UTR leader | A-start | AGGAAA |
| 55 | 5′UTR (DNA) | 5′v1.0 Core | TAAGAGAGAAAAGAAGAGTAAGAAGAAATATAAG |
| | | | A |
| 56 | 5′UTR (RNA) | 5′v1.0 | GGGAAAUAAGAGAGAAAAGAAGAGUAAGAAGAAA |
| | | Minus Kozak | UAUAAGA |
| 57 | 5′UTR (RNA) | 5′v1.0 | UAAGAGAGAAAAGAAGAGUAAGAAGAAAUAUAAG |
| | | Minus Leader | AGCCACC |
| 58 | 5′UTR (DNA) | 5′v1.0 | GGGAAATAAGAGAGAAAAGAAGAGTAAGAAGAAA |
| | | | TATAAGAGCCACC |
| 59 | 5′UTR (RNA) | 5′v1.0 | AGGAAAUAAGAGAGAAAAGAAGAGUAAGAAGAAA |
| | | A-start | UAUAAGAGCCACC |
| 60 | 5′UTR (RNA) | 5′v1.1 | AGGAAAUAAGAGAGAAAAGAAGAGUAAGAAGAAA |
| | | (Vl) A-start | UAUAAGACCCCGGCGCCGCCACC |
| 61 | 5′UTR (RNA) | 5′v1.1 | UAAGAGAGAAAAGAAGAGUAAGAAGAAAUAUAAG |
| | | (Vl) minus | ACCCCGGCGCCGCCACC |
| | | leader | |
| 62 | 5′UTR (RNA) | V2-5′UTR | GGGAAAUAAGAGAGAAAAGAAGAGUAAGAAGAAA |
| | | (V2) | UAUAAGACCCCGGCGCCACC |
| 63 | 5′UTR (RNA) | V2-5′UTR | AGGAAAUAAGAGAGAAAAGAAGAGUAAGAAGAAA |
| | | A-start | UAUAAGACCCCGGCGCCACC |
| 64 | 5′UTR (RNA) | V2-5′UTR | UAAGAGAGAAAAGAAGAGUAAGAAGAAAUAUAAG |
| | | minus leader | ACCCCGGCGCCACC |
| 65 | 5′UTR (RNA) | CG1-5′UTR | GGGAAAUAAGAGAGAAAAGAAGAGUAAGAAGAAA |
| | | | UAUAAGAGCGCCCCGCGGCGCCCCGCGGCCACC |
| 66 | 5′UTR (RNA) | CG1-5′UTR | AGGAAAUAAGAGAGAAAAGAAGAGUAAGAAGAAA |
| | | A-start | UAUAAGAGCGCCCCGCGGCGCCCCGCGGCCACC |
| 67 | 5′UTR (RNA) | CG1-5′UTR | UAAGAGAGAAAAGAAGAGUAAGAAGAAAUAUAAG |
| | | Minus leader | AGCGCCCCGCGGCGCCCCGCGGCCACC |
| 68 | 5′UTR (RNA) | CG2-5′UTR | GGGAAAUAAGAGAGAAAAGAAGAGUAAGAAGAAA |
| | | | UAUAAGACCCGCCCGCCCCGCCCCGCCGCCACC |
| 69 | 3′UTR (DNA) | 3′v1.1 | TGATAATAGGCTGGAGCCTCGGTGGCCTAGCTTC |
| | | | TTGCCCCTTGGGCCTCCCCCCAGCCCCTCCTCCC |
| | | | CTTCCTGCACCCGTACCCCCGTGGTCTTTGAATA |
| | | | AAGTCTGAGTGGGCGGC |
| 70 | 3′UTR (RNA) | 3′v1.1 | UGAUAAUAGGCUGGAGCCUCGGUGGCCUAGCUUC |
| | | | UUGCCCCUUGGGCCUCCCCCCAGCCCCUCCUCCC |
| | | | CUUCCUGCACCCGUACCCCCGUGGUCUUUGAAUA |
| | | | AAGUCUGAGUGGGCGGC |
| 71 | 3′UTR (DNA) | hOxa1_3′UTR | TGACTTAGTTCTGTGCGCATTCTGGCAGGAATTC |
| | | | TGTCTCTTCAGAGACTCATCCTCAAAACAAGACT |
| | | | TGACACTGTGTCCTTGCCCCAGTCCTAGGAACTG |
| | | | TGGCACACAGAGTAGTTCATTTTAAAAACGGATT |
| | | | ACTAGAAACACTCTTGTACTTTAGTTTATAAGAG |
| | | | AGCACTGGGTAGCCAAGTGATCTTCCCATTCACA |
| | | | GAGTTAGTAAACCTCTGTACTACTAGCTG |
| 72 | 3′UTR (RNA) | hOxa1_3′UTR | UGACUUAGUUCUGUGCGCAUUCUGGCAGGAAUUC |
| | | | UGUCUCUUCAGAGACUCAUCCUCAAAACAAGACU |
| | | | UGACACUGUGUCCUUGCCCCAGUCCUAGGAACUG |
| | | | UGGCACACAGAGUAGUUCAUUUUAAAAACGGAUU |
| | | | ACUAGAAACACUCUUGUACUUUAGUUUAUAAGAG |
| | | | AGCACUGGGUAGCCAAGUGAUCUUCCCAUUCACA |
| | | | GAGUUAGUAAACCUCUGUACUACUAGCUG |
| 73 | 3′UTR (DNA) | hOxa1_short | TGAACTGTGGCACACAGAGTAGTTCATTTTAAAA |
| | | 3 ′UTR | ACGGATTTCGATAAACACTCTTGTACTTTAGTTT |
| | | | ATAAGAGAGCACTGGGTAGCCAAGTGATCTTCCC |
| | | | ATTCACAGAGTTAGTAAACCTCTGTACTACTAGC |
| | | | TG |
| 74 | 3′UTR (RNA) | hOxal_short | UGAACUGUGGCACACAGAGUAGUUCAUUUUAAAA |
| | | 3 ′UTR | ACGGAUUUCGAUAAACACUCUUGUACUUUAGUUU |
| | | | AUAAGAGAGCACUGGGUAGCCAAGUGAUCUUCCC |
| | | | AUUCACAGAGUUAGUAAACCUCUGUACUACUAGC |
| | | | UG |
| 75 | 3′UTR (DNA) | mt-rpS12Al 3UTR | TGACAGAAGAAGTGACGGCTGGGGGCACAGTGGG |
| | | | CTGGGCGCCCCTGCAGAACTAGAACCTTCCGCTC |
| | | | CTGGCTGCCACAGGGTCCTCCGTAGCTGGCCTTT |
| | | | GCGCCTGTAGAGGCAGCCACTCTAGGATTCAAGT |
| | | | CCTGGCTCCGCCTCTTCCATCAGGACCACTA |
| 76 | 3′UTR (RNA) | mt-rpS12Al 3UTR | UGACAGAAGAAGUGACGGCUGGGGGCACAGUGGG |
| | | | CUGGGCGCCCCUGCAGAACUAGAACCUUCCGCUC |
| | | | CUGGCUGCCACAGGGUCCUCCGUAGCUGGCCUUU |
| | | | GCGCCUGUAGAGGCAGCCACUCUAGGAUUCAAGU |
| | | | CCUGGCUCCGCCUCUUCCAUCAGGACCACUA |
| 77 | 3′UTR (DNA) | mSOD2 3UTR (DNA) | TGAACCTCACTCACGGCCACATTGAGTGCCAGGC |
| | | | TCCGGGCTGGTTTATAGTAGTGTAGAGCATTGCA |
| | | | GCACTTAGACTGGGGTGCTGTAGTCTTTATTGTA |
| | | | GTCTTTCCACATACCTGATAATTCTTAGATAATT |
| | | | TCTTATTTTAATTAAATCTATTCTTAGGCT |
| 78 | 3′UTR (RNA) | mSOD2 3UTR (RNA) | UGAACCUCACUCACGGCCACAUUGAGUGCCAGGC |
| | | | UCCGGGCUGGUUUAUAGUAGUGUAGAGCAUUGCA |
| | | | GCACUUAGACUGGGGUGCUGUAGUCUUUAUUGUA |
| | | | GUCUUUCCACAUACCUGAUAAUUCUUAGAUAAUU |
| | | | UCUUAUUUUAAUUAAAUCUAUUCUUAGGCU |
| 79 | 5′UTR (RNA) | CG2-5′UTR | AGGAAAUAAGAGAGAAAAGAAGAGUAAGAAGAAA |
| | | A-start | UAUAAGACCCGCCCGCCCCGCCCCGCCGCCACC |
| 80 | 5′UTR (RNA) | CG2-5′UTR | UAAGAGAGAAAAGAAGAGUAAGAAGAAAUAUAAG |
| | | Minus leader | ACCCGCCCGCCCCGCCCCGCCGCCACC |
| 81 | 5′UTR | KT1-UTR | AGGCCCGCCGCCAAC |
| | | A-start | |
| 82 | 5′UTR | KT2-UTR | AGGCCCGCCGCCACC |
| | | A-start | |
| 83 | 5′UTR | KT3-UTR | AGGCCCGCCGCCGAC |
| | | A-start | |
| 84 | 5′UTR | KT4-UTR | AGGCCCGCCGCCGCC |
| | | A-start | |
| 85 | 5′UTR | GCC3-ExtKozak | AGGAAA GCCGCCGCCGGCACC |
| | | A-start | |
| 86 | 5′UTR | GCC3-ExtKozak | GCCGCCGCCGCCACC |
| | | Minus leader | |
| 87 | 5′UTR (DNA) | S065 core | CCTCATATCCAGGCTCAAGAATAGAGCTCAGTGT |
| | | | TTTGTTGTTTAATCATTCCGACGTGTTTTGCGAT |
| | | | ATTCGCGCAAAGCAGCCAGTCGCGCGCTTGCTTT |
| | | | TAAGTAGAGTTGTTTTTCCACCCGTTTGCCAGGC |
| | | | ATCTTTAATTTAACATATTTTTATTTTTCAGGCT |
| | | | AACCTA |
| 88 | 5′UTR (DNA) | 3065 | GGGAGACCTCATATCCAGGCTCAAGAATAGAGCT |
| | | | CAGTGTTTTGTTGTTTAATCATTCCGACGTGTTT |
| | | | TGCGATATTCGCGCAAAGCAGCCAGTCGCGCGCT |
| | | | TGCTTTTAAGTAGAGTTGTTTTTCCACCCGTTTG |
| | | | CCAGGCATCTTTAATTTAACATATTTTTATTTTT |
| | | | CAGGCTAACCTAAAGCAGAGAA |
| 89 | 5′UTR (RNA) | 3065 | AGGAGACCUCAUAUCCAGGCUCAAGAAUAGAGCU |
| | | A-start | CAGUGUUUUGUUGUUUAAUCAUUCCGACGUGUUU |
| | | | UGCGAUAUUCGCGCAAAGCAGCCAGUCGCGCGCU |
| | | | UGCUUUUAAGUAGAGUUGUUUUUCCACCCGUUUG |
| | | | CCAGGCAUCUUUAAUUUAACAUAUUUUUAUUUUU |
| | | | CAGGCUAACCUAAAGCAGAGAA |
| 90 | 5′UTR (RNA) | S065 core | CCUCAUAUCCAGGCUCAAGAAUAGAGCUCAGUGU |
| | | Minus leader | UUUGUUGUUUAAUCAUUCCGACGUGUUUUGCGAU |
| | | | AUUCGCGCAAAGCAGCCAGUCGCGCGCUUGCUUU |
| | | | UAAGUAGAGUUGUUUUUCCACCCGUUUGCCAGGC |
| | | | AUCUUUAAUUUAACAUAUUUUUAUUUUUCAGGCU |
| | | | AACCUAAAGCAGAGAA |
| 91 | 5′UTR (DNA) | combo3_S065 | GGGAGACCTCATATCCAGGCTCAAGAATAGAGCT |
| | | (3065 core | CAGTGTTTTGTTGTTTAATCATTCCGACGTGTTT |
| | | extended Kozak) | TGCGATATTCGCGCAAAGCAGCCAGTCGCGCGCT |
| | | | TGCTTTTAAGTAGAGTTGTTTTTCCACCCGTTTG |
| | | | CCAGGCATCTTTAATTTAACATATTTTTATTTTT |
| | | | CAGGCTAACCTACGCCGCCACC |
| 92 | 5′UTR (RNA) | combo3_S065 | AGGAGACCUCAUAUCCAGGCUCAAGAAUAGAGCU |
| | | (3065 core | CAGUGUUUUGUUGUUUAAUCAUUCCGACGUGUUU |
| | | extended Kozak) | UGCGAUAUUCGCGCAAAGCAGCCAGUCGCGCGCU |
| | | A-start | UGCUUUUAAGUAGAGUUGUUUUUCCACCCGUUUG |
| | | | CCAGGCAUCUUUAAUUUAACAUAUUUUUAUUUUU |
| | | | CAGGCUAACCUACGCCGCCACC |
| 93 | 5′UTR (RNA) | combo3_S065 | CCUCAUAUCCAGGCUCAAGAAUAGAGCUCAGUGU |
| | | (3065 core | UUUGUUGUUUAAUCAUUCCGACGUGUUUUGCGAU |
| | | extended Kozak) | AUUCGCGCAAAGCAGCCAGUCGCGCGCUUGCUUU |
| | | Minus leader | UAAGUAGAGUUGUUUUUCCACCCGUUUGCCAGGC |
| | | | AUCUUUAAUUUAACAUAUUUUUAUUUUUCAGGCU |
| | | | AACCUACGCCGCCACC |
| 94 | 5′UTR (DNA) | combol_S065 | GGGAAACCCCCCACCCCCGCCTCATATCCAGGCT |
| | | (CR3) | CAAGAATAGAGCTCAGTGTTTTGTTGTTTAATCA |
| | | | TTCCGACGTGTTTTGCGATATTCGCGCAAAGCAG |
| | | | CCAGTCGCGCGCTTGCTTTTAAGTAGAGTTGTTT |
| | | | TTCCACCCGTTTGCCAGGCATCTTTAATTTAACA |
| | | | TATTTTTATTTTTCAGGCTAACCTAAAGCAGAGA |
| | | | A |
| 95 | 5′UTR (RNA) | combol_S065 | AGGAAACCCCCCACCCCCGCCUCAUAUCCAGGCU |
| | | (CR3) | CAAGAAUAGAGCUCAGUGUUUUGUUGUUUAAUCA |
| | | A-start | UUCCGACGUGUUUUGCGAUAUUCGCGCAAAGCAG |
| | | | CCAGUCGCGCGCUUGCUUUUAAGUAGAGUUGUUU |
| | | | UUCCACCCGUUUGCCAGGCAUCUUUAAUUUAACA |
| | | | UAUUUUUAUUUUUCAGGCUAACCUAAAGCAGAGA |
| | | | A |
| 96 | 5′UTR (RNA) | combol_S065 | CCCCCCACCCCCGCCUCAUAUCCAGGCUCAAGAA |
| | | (CR3) | UAGAGCUCAGUGUUUUGUUGUUUAAUCAUUCCGA |
| | | Minus leader | CGUGUUUUGCGAUAUUCGCGCAAAGCAGCCAGUC |
| | | | GCGCGCUUGCUUUUAAGUAGAGUUGUUUUUCCAC |
| | | | CCGUUUGCCAGGCAUCUUUAAUUUAACAUAUUUU |
| | | | UAUUUUUCAGGCUAACCUAAAGCAGAGAA |
| 97 | 5′UTR (DNA) | combo2_S065 | GGGAAATCCCCACAACCGCCTCATATCCAGGCTC |
| | | (CR5) | AAGAATAGAGCTCAGTGTTTTGTTGTTTAATCAT |
| | | | TCCGACGTGTTTTGCGATATTCGCGCAAAGCAGC |
| | | | CAGTCGCGCGCTTGCTTTTAAGTAGAGTTGTTTT |
| | | | TCCACCCGTTTGCCAGGCATCTTTAATTTAACAT |
| | | | ATTTTTATTTTTCAGGCTAACCTAAAGCAGAGAA |
| 98 | 5′UTR (RNA) | combo2_S065 | AGGAAAUCCCCACAACCGCCUCAUAUCCAGGCUC |
| | | (CR5) | AAGAAUAGAGCUCAGUGUUUUGUUGUUUAAUCAU |
| | | A-start | UCCGACGUGUUUUGCGAUAUUCGCGCAAAGCAGC |
| | | | CAGUCGCGCGCUUGCUUUUAAGUAGAGUUGUUUU |
| | | | UCCACCCGUUUGCCAGGCAUCUUUAAUUUAACAU |
| | | | AUUUUUAUUUUUCAGGCUAACCUAAAGCAGAGAA |
| 99 | 5′UTR (RNA) | combo2_S065 | UCCCCACAACCGCCUCAUAUCCAGGCUCAAGAAU |
| | | (CR5) | AGAGCUCAGUGUUUUGUUGUUUAAUCAUUCCGAC |
| | | Minus leader | GUGUUUUGCGAUAUUCGCGCAAAGCAGCCAGUCG |
| | | | CGCGCUUGCUUUUAAGUAGAGUUGUUUUUCCACC |
| | | | CGUUUGCCAGGCAUCUUUAAUUUAACAUAUUUUU |
| | | | AUUUUUCAGGCUAACCUAAAGCAGAGAA |
| 100 | 5′UTR (DNA) | combo4_S065 | GGGAAACCCCCCACCCCCGCCTCATATCCAGGCT |
| | | (CR3) | CAAGAATAGAGCTCAGTGTTTTGTTGTTTAATCA |
| | | | TTCCGACGTGTTTTGCGATATTCGCGCAAAGCAG |
| | | | CCAGTCGCGCGCTTGCTTTTAAGTAGAGTTGTTT |
| | | | TTCCACCCGTTTGCCAGGCATCTTTAATTTAACA |
| | | | TATTTTTATTTTTCAGGCTAACCTACGCCGCCAC |
| | | | C |
| 101 | 5′UTR (RNA) | combo4_S065 | AGGAAACCCCCCACCCCCGCCUCAUAUCCAGGCU |
| | | (CR3) | CAAGAAUAGAGCUCAGUGUUUUGUUGUUUAAUCA |
| | | A-start | UUCCGACGUGUUUUGCGAUAUUCGCGCAAAGCAG |
| | | | CCAGUCGCGCGCUUGCUUUUAAGUAGAGUUGUUU |
| | | | UUCCACCCGUUUGCCAGGCAUCUUUAAUUUAACA |
| | | | UAUUUUUAUUUUUCAGGCUAACCUACGCCGCCAC |
| | | | C |
| 102 | 5′UTR (RNA) | combo4_S065 | CCCCCCACCCCCGCCUCAUAUCCAGGCUCAAGAA |
| | | (CR3) | UAGAGCUCAGUGUUUUGUUGUUUAAUCAUUCCGA |
| | | Minus leader | CGUGUUUUGCGAUAUUCGCGCAAAGCAGCCAGUC |
| | | | GCGCGCUUGCUUUUAAGUAGAGUUGUUUUUCCAC |
| | | | CCGUUUGCCAGGCAUCUUUAAUUUAACAUAUUUU |
| | | | UAUUUUUCAGGCUAACCUACGCCGCCACC |
| 103 | 5′UTR (DNA) | F153, | GGGAAATCCCCACAACCGCCTCATATCCAGGCTC |
| | | combo5_S065 | AAGAATAGAGCTCAGTGTTTTGTTGTTTAATCAT |
| | | (CR5) | TCCGACGTGTTTTGCGATATTCGCGCAAAGCAGC |
| | | | CAGTCGCGCGCTTGCTTTTAAGTAGAGTTGTTTT |
| | | | TCCACCCGTTTGCCAGGCATCTTTAATTTAACAT |
| | | | ATTTTTATTTTTCAGGCTAACCTACGCCGCCACC |
| 104 | 5′UTR (RNA) | F153, | AGGAAAUCCCCACAACCGCCUCAUAUCCAGGCUC |
| | | combo5_S065 | AAGAAUAGAGCUCAGUGUUUUGUUGUUUAAUCAU |
| | | (CR5) | UCCGACGUGUUUUGCGAUAUUCGCGCAAAGCAGC |
| | | A-start | CAGUCGCGCGCUUGCUUUUAAGUAGAGUUGUUUU |
| | | | UCCACCCGUUUGCCAGGCAUCUUUAAUUUAACAU |
| | | | AUUUUUAUUUUUCAGGCUAACCUACGCCGCCACC |
| 105 | 5′UTR (RNA) | F153, | UCCCCACAACCGCCUCAUAUCCAGGCUCAAGAAU |
| | | combo5_S065 | AGAGCUCAGUGUUUUGUUGUUUAAUCAUUCCGAC |
| | | (CR5) | GUGUUUUGCGAUAUUCGCGCAAAGCAGCCAGUCG |
| | | Minus leader | CGCGCUUGCUUUUAAGUAGAGUUGUUUUUCCACC |
| | | | CGUUUGCCAGGCAUCUUUAAUUUAACAUAUUUUU |
| | | | AUUUUUCAGGCUAACCUACGCCGCCACC |
| 106 | 5′UTR (DNA) | combo1_V1.1 | GGGAAACCCCCCACCCCCGGGGAAATAAGAGAGA |
| | | (CR3, V1) | AAAGAAGAGTAAGAAGAAATATAAGACCCCGGCG |
| | | | CCGCCACC |
| 107 | 5′UTR (RNA) | combo1_V1.1 | AGGAAACCCCCCACCCCCGGGGAAAUAAGAGAGA |
| | | (CR3, V1) | AAAGAAGAGUAAGAAGAAAUAUAAGACCCCGGCG |
| | | A-start | CCGCCACC |
| 108 | 5′UTR (RNA) | combo1_V1.1 | CCCCCCACCCCCGGGGAAAUAAGAGAGAAAAGAA |
| | | (CR3, V1) | GAGUAAGAAGAAAUAUAAGACCCCGGCGCCGCCA |
| | | Minus leader | CC |
| 109 | 5′UTR (DNA) | combo2_V1.1 | GGGAAATCCCCACAACCGGGGAAATAAGAGAGAA |
| | | (CR5, V1) | AAGAAGAGTAAGAAGAAATATAAGACCCCGGCGC |
| | | | CGCCACC |
| 110 | 5′UTR (RNA) | combo2_V1.1 | AGGAAAUCCCCACAACCGGGGAAAUAAGAGAGAA |
| | | (CR5, V1) | AAGAAGAGUAAGAAGAAAUAUAAGACCCCGGCGC |
| | | A-start | CGCCACC |
| 111 | 5′UTR (RNA) | combo2_V1.1 | UCCCCACAACCGGGGAAAUAAGAGAGAAAAGAAG |
| | | (CR5, VI) | AGUAAGAAGAAAUAUAAGACCCCGGCGCCGCCAC |
| | | Minus leader | C |
| 112 | 5′UTR (DNA) | CrichCR4 + GCC3- | GGGAAACCCCCCTAAGCCGCCGCCGCCGCCACC |
| | | ExtKozak | |
| | | (CR4, GCC3) | |
| 113 | 5′UTR (RNA) | CrichCR4 + GCC3- | AGGAAACCCCCCUAAGCCGCCGCCGCCGCCACC |
| | | ExtKozak | |
| | | (CR4, GCC3) | |
| | | A-start | |
| 114 | 5′UTR (RNA) | CrichCR4 + GCC3- | CCCCCCUAAGCCGCCGCCGCCGCCACC |
| | | ExtKozak | |
| | | (CR4, GCC3) | |
| | | Minus leader | |
| 115 | 5′UTR (DNA) | F856, RNAseP_p1 | GGGAAATAAGAGAGAAAAGAAGAGTAAGAAGAAA |
| | | (V1, RNAseP) | TATAAGATCTCCCTGAGCTTCAGGGAGCCCCGGC |
| | | | GCCGCCACC |
| 116 | 5′UTR (RNA) | F856, RNAseP_p1 | GGGAAAUAAGAGAGAAAAGAAGAGUAAGAAGAAA |
| | | (V1, RNAseP) | UAUAAGAUCUCCCUGAGCUUCAGGGAGCCCCGGC |
| | | | GCCGCCACC |
| 117 | 5′UTR (RNA) | RNAseP_p1 | AGGAAAUAAGAGAGAAAAGAAGAGUAAGAAGAAA |
| | | (V1, RNAseP) | UAUAAGAUCUCCCUGAGCUUCAGGGAGCCCCGGC |
| | | A-start | GCCGCCACC |
| 118 | 5′UTR (RNA) | RNAseP_p1 | UAAGAGAGAAAAGAAGAGUAAGAAGAAAUAUAAG |
| | | (V1, RNAseP) | AUCUCCCUGAGCUUCAGGGAGCCCCGGCGCCGCC |
| | | Minus leader | ACC |
| 119 | 5′UTR (DNA) | RNAse P-2 | GGGAAATCTCCCTGAGCTTCAGGGAGTAAGAGAG |
| | | (V1, RNAseP) | AAAAGAAGAGTAAGAAGAAATATAAGACCCCGGC |
| | | | GCCGCCACC |
| 120 | 5′UTR (RNA) | RNAse P-2 | GGGAAAUCUCCCUGAGCUUCAGGGAGUAAGAGAG |
| | | (V1, RNAseP) | AAAAGAAGAGUAAGAAGAAAUAUAAGACCCCGGC |
| | | | GCCGCCACC |
| 121 | 5′UTR (RNA) | RNAse P-2 | AGGAAAUCUCCCUGAGCUUCAGGGAGUAAGAGAG |
| | | (V1, RNAseP) | AAAAGAAGAGUAAGAAGAAAUAUAAGACCCCGGC |
| | | A-start | GCCGCCACC |
| 122 | 5′UTR (RNA) | RNAse P-2 | UCUCCCUGAGCUUCAGGGAGUAAGAGAGAAAAGA |
| | | (V1, RNAseP) | AGAGUAAGAAGAAAUAUAAGACCCCGGCGCCGCC |
| | | Minus leader | ACC |
| 123 | 5′UTR (DNA) | RNAseP_p3 | GGGAAATAAGAGAGAAAAGAAGATCTCCCTGAGC |
| | | (V1, RNAseP) | TTCAGGGAGGTAAGAAGAAATATAAGACCCCGGC |
| | | | GCCGCCACC |
| 124 | 5′UTR (RNA) | RNAseP_p3 | GGGAAAUAAGAGAGAAAAGAAGAUCUCCCUGAGC |
| | | (V1, RNAseP) | UUCAGGGAGGUAAGAAGAAAUAUAAGACCCCGGC |
| | | | GCCGCCACC |
| 125 | 5′UTR (RNA) | RNAseP_p3 | AGGAAAUAAGAGAGAAAAGAAGAUCUCCCUGAGC |
| | | (V1, RNAseP) | UUCAGGGAGGUAAGAAGAAAUAUAAGACCCCGGC |
| | | A-start | GCCGCCACC |
| 126 | 5′UTR (RNA) | RNAseP_p3 | UAAGAGAGAAAAGAAGAUCUCCCUGAGCUUCAGG |
| | | (V1, RNAseP) | GAGGUAAGAAGAAAUAUAAGACCCCGGCGCCGCC |
| | | Minus leader | ACC |
| 127 | 5′UTR (DNA) | F593, | GGGAAACCCCCCACCCCCGTAAGAGAGAAAAGAA |
| | | combo1_P2_p1 | GAGTAAGAAGAAATATAAGATCTCCCTGAGCTTC |
| | | (V1, CR3, | AGGGAGCCCCGGCGCCGCCACC |
| | | RNAseP) | |
| 128 | 5′UTR (RNA) | F593, | GGGAAACCCCCCACCCCCGUAAGAGAGAAAAGAA |
| | | combo1_P2_p1 | GAGUAAGAAGAAAUAUAAGAUCUCCCUGAGCUUC |
| | | (V1, CR3, | AGGGAGCCCCGGCGCCGCCACC |
| | | RNAseP) | |
| 129 | 5′UTR (RNA) | F593, | AGGAAACCCCCCACCCCCGUAAGAGAGAAAAGAA |
| | | combo1_P2_p1 | GAGUAAGAAGAAAUAUAAGAUCUCCCUGAGCUUC |
| | | (V1, CR3, | AGGGAGCCCCGGCGCCGCCACC |
| | | RNAseP) | |
| | | A-start | |
| 130 | 5′UTR (RNA) | F593, | CCCCCCACCCCCGUAAGAGAGAAAAGAAGAGUAA |
| | | combo1_P2_p1 | GAAGAAAUAUAAGAUCUCCCUGAGCUUCAGGGAG |
| | | (V1, CR3, | CCCCGGCGCCGCCACC |
| | | RNAseP) | |
| | | Minus leader | |
| 131 | 5′UTR (DNA) | combo1_P2_p2 | GGGAAACCCCCCACCCCCGTCTCCCTGAGCTTCA |
| | | (V1, CR3, | GGGAGTAAGAGAGAAAAGAAGAGTAAGAAGAAAT |
| | | RNAseP) | ATAAGACCCCGGCGCCGCCACC |

| | | | |
|---|---|---|---|
| 132 | 5′UTR (RNA) | combo1_P2_p2 | GGGAAACCCCCCACCCCCGUCUCCCUGAGCUUCA |
| | | (V1, CR3, | GGGAGUAAGAGAGAAAAGAAGAGUAAGAAGAAAU |
| | | RNAseP) | AUAAGACCCCGGCGCCGCCACC |
| 133 | 5′UTR (RNA) | combo1_P2_p2 | AGGAAACCCCCCACCCCCGUCUCCCUGAGCUUCA |
| | | (V1, CR3, | GGGAGUAAGAGAGAAAAGAAGAGUAAGAAGAAAU |
| | | RNAseP) | AUAAGACCCCGGCGCCGCCACC |
| | | A-start | |
| 134 | 5′UTR (RNA) | combol_P2_p2 | CCCCCCACCCCCGUCUCCCUGAGCUUCAGGGAGU |
| | | (V1, CR3, | AAGAGAGAAAAGAAGAGUAAGAAGAAAUAUAAGA |
| | | RNAseP) | CCCCGGCGCCGCCACC |
| | | Minus leader | |
| 135 | 5′UTR (DNA) | combo1_P2_p3 | GGGAAACCCCCCACCCCCGTAAGAGAGAAAAGAA |
| | | (V1, CR3, | GATCTCCCTGAGCTTCAGGGAGGTAAGAAGAAAT |
| | | RNAseP) | ATAAGACCCCGGCGCCGCCACC |
| 136 | 5′UTR (RNA) | combo1_P2_p3 | GGGAAACCCCCCACCCCCGUAAGAGAGAAAAGAA |
| | | (V1, CR3, | GAUCUCCCUGAGCUUCAGGGAGGUAAGAAGAAAU |
| | | RNAseP) | AUAAGACCCCGGCGCCGCCACC |
| 137 | 5′UTR (RNA) | combo1_P2_p3 | AGGAAACCCCCCACCCCCGUAAGAGAGAAAAGAA |
| | | (V1, CR3, | GAUCUCCCUGAGCUUCAGGGAGGUAAGAAGAAAU |
| | | RNAseP) | AUAAGACCCCGGCGCCGCCACC |
| | | A-start | |
| 138 | 5′UTR (RNA) | combo1_P2_p3 | CCCCCCACCCCCGUAAGAGAGAAAAGAAGAUCUC |
| | | (V1, CR3, | CCUGAGCUUCAGGGAGGUAAGAAGAAAUAUAAGA |
| | | RNAseP) | CCCCGGCGCCGCCACC |
| | | Minus leader | |
| 139 | 5′UTR (DNA) | combo2_P2_p2 | GGGAAATCCCCACAACCGTCTCCCTGAGCTTCAG |
| | | (V1, CR5, | GGAGTAAGAGAGAAAAGAAGAGTAAGAAGAAATA |
| | | RNAseP) | TAAGACCCCGGCGCCGCCACC |
| 140 | 5′UTR (RNA) | combo2_P2_p2 | GGGAAAUCCCCACAACCGUCUCCCUGAGCUUCAG |
| | | (V1, CR5, | GGAGUAAGAGAGAAAAGAAGAGUAAGAAGAAAUA |
| | | RNAseP) | UAAGACCCCGGCGCCGCCACC |
| 141 | 5′UTR (RNA) | combo2_P2_p2 | AGGAAAUCCCCACAACCGUCUCCCUGAGCUUCAG |
| | | (V1, CR5, | GGAGUAAGAGAGAAAAGAAGAGUAAGAAGAAAUA |
| | | RNAseP) | UAAGACCCCGGCGCCGCCACC |
| | | A-start | |
| 142 | 5′UTR (RNA) | combo2_P2_p2 | UCCCCACAACCGUCUCCCUGAGCUUCAGGGAGUA |
| | | (V1, CR5, | AGAGAGAAAAGAAGAGUAAGAAGAAAUAUAAGAC |
| | | RNAseP) | CCCGGCGCCGCCACC |
| | | Minus leader | |
| 143 | 5′UTR (DNA) | combo2_P2_p3 | GGGAAATCCCCACAACCGTAAGAGAGAAAAGAAG |
| | | (V1, CR5, | ATCTCCCTGAGCTTCAGGGAGGTAAGAAGAAATA |
| | | RNAseP) | TAAGACCCCGGCGCCGCCACC |
| 144 | 5′UTR (RNA) | combo2_P2_p3 | GGGAAAUCCCCACAACCGUAAGAGAGAAAAGAAG |
| | | (V1, CR5, | AUCUCCCUGAGCUUCAGGGAGGUAAGAAGAAAUA |
| | | RNAseP) | UAAGACCCCGGCGCCGCCACC |
| 145 | 5′UTR (RNA) | combo2_P2_p3 | AGGAAAUCCCCACAACCGUAAGAGAGAAAAGAAG |
| | | (V1, CR5, | AUCUCCCUGAGCUUCAGGGAGGUAAGAAGAAAUA |
| | | RNAseP) | UAAGACCCCGGCGCCGCCACC |
| | | A-start | |
| 146 | 5′UTR (RNA) | combo2_P2_p3 | UCCCCACAACCGUAAGAGAGAAAAGAAGAUCUCC |
| | | (V1, CR5, | CUGAGCUUCAGGGAGGUAAGAAGAAAUAUAAGAC |
| | | RNAseP) | CCCGGCGCCGCCACC |
| | | Minus leader | |
| 147 | 5′UTR (DNA) | mt-rpS12 5UTR | GGGAGCTGGATTCAGCGTGTCCGCGACCTCACCT |
| | | | TTAGGTCCTGTGAGGTCGGTGGAATCCTGGGGTC |
| | | | CTCCAAATCTACCAGGCCATCTCCCCAGTTTCCC |
| | | | AGTTCTTCCTGCGTGCGGGCGAGAGTGGTTGGGC |
| | | | CCTCGGGAACCCACTCAGAGCGAGGCTAAATTTA |
| | | | CGGAGGGACTTTCTGTTAGCAGCATGAGGGCCTG |

| | | | |
|---|---|---|---|
| | | | TGGTTAGACCTATAGAGGGACGGCCCAGGTGGCA |
| | | | GG |
| 148 | 5′UTR (RNA) | mt-rpS12 5UTR | GGGAGCUGGAUUCAGCGUGUCCGCGACCUCACCU |
| | | | UUAGGUCCUGUGAGGUCGGUGGAAUCCUGGGGUC |
| | | | CUCCAAAUCUACCAGGCCAUCUCCCCAGUUUCCC |
| | | | AGUUCUUCCUGCGUGCGGGCGAGAGUGGUUGGGC |
| | | | CCUCGGGAACCCACUCAGAGCGAGGCUAAAUUUA |
| | | | CGGAGGGACUUUCUGUUAGCAGCAUGAGGGCCUG |
| | | | UGGUUAGACCUAUAGAGGGACGGCCCAGGUGGCA |
| | | | GG |
| 149 | 3′UTR (DNA) | 3′v1.0 | TGATAATAGGCTGGAGCCTCGGTGGCCATGCTTC |
| | | | TTGCCCCTTGGGCCTCCCCCCAGCCCCTCCTCCC |
| | | | CTTCCTGCACCCGTACCCCCGTGGTCTTTGAATA |
| | | | AAGTCTGAGTGGGCGGC |
| 150 | 3′UTR (RNA) | 3′v1.0 | UGAUAAUAGGCUGGAGCCUCGGUGGCCAUGCUUC |
| | | | UUGCCCCUUGGGCCUCCCCCCAGCCCCUCCUCCC |
| | | | CUUCCUGCACCCGUACCCCCGUGGUCUUUGAAUA |
| | | | AAGUCUGAGUGGGCGGC |
| 151 | 3′UTR (RNA) | 3′v1.0 | GCUGGAGCCUCGGUGGCCAUGCUUCUUGCCCCUU |
| | | Minus stop | GGGCCUCCCCCCAGCCCCUCCUCCCCUUCCUGCA |
| | | | CCCGUACCCCCGUGGUCUUUGAAUAAAGUCUGAG |
| | | | UGGGCGGC |
| 152 | 3′UTR (RNA) | 3′v1.0 | UAAUAGUAAGCUGGAGCCUCGGUGGCCAUGCUUC |
| | | altered stop | UUGCCCCUUGGGCCUCCCCCCAGCCCCUCCUCCC |
| | | | CUUCCUGCACCCGUACCCCCGUGGUCUUUGAAUA |
| | | | AAGUCUGAGUGGGCGGC |
| 153 | 3′UTR (RNA) | 3′v1.1 | GCUGGAGCCUCGGUGGCCUAGCUUCUUGCCCCUU |
| | | Minus stop | GGGCCUCCCCCCAGCCCCUCCUCCCCUUCCUGCA |
| | | | CCCGUACCCCCGUGGUCUUUGAAUAAAGUCUGAG |
| | | | UGGGCGGC |
| 154 | 3′UTR (RNA) | 3′v1l.1 | UAAUAGUAAGCUGGAGCCUCGGUGGCCUAGCUUC |
| | | altered stop | UUGCCCCUUGGGCCUCCCCCCAGCCCCUCCUCCC |
| | | | CUUCCUGCACCCGUACCCCCGUGGUCUUUGAAUA |
| | | | AAGUCUGAGUGGGCGGC |
| 155 | Epitope Tag | FLAG | DYKDDDDK |
| 156 | Epitope Tag | 3XFLAG | DYKDHDGDYKDHDIDYKDDDK |
| 157 | miR | miR-122 | CCUUAGCAGAGCUGUGGAGUGUGACAAUGGUGUU |
| | | | UGUGUCUAAACUAUCAAACGCCAUUAUCACACUA |
| | | | AAUAGCUACUGCUAGGC |
| 158 | miR | miR-122-3p | AACGCCAUUAUCACACUAAAUA |
| 159 | miR binding | miR-122-3p | UAUUUAGUGUGAUAAUGGCGUU |
| | site | binding site | |
| 160 | miR | miR-122-5p | UGGAGUGUGACAAUGGUGUUUG |
| 161 | miR binding | miR-122-5p | CAAACACCAUUGUCACACUCCA |
| | site | binding site | |
| 162 | 3′UTR (DNA) | 3′v1.1m142 | TGATAATAGGCTGGAGCCTCGGTGGCCTAGCTTC |
| | | 3′v1.1 + miR- | TTGCCCCTTGGGCCTCCCCCCAGCCCCTCCTCCC |
| | | 142-3p binding | CTTCCTGCACCCGTACCCCCTCCATAAAGTAGGA |
| | | site | AACACTACAGTGGTCTTTGAATAAAGTCTGAGTG |
| | | | GGCGGC |
| 163 | 3′UTR (RNA) | 3′v1.1m142 | UGAUAAUAGGCUGGAGCCUCGGUGGCCUAGCUUC |
| | | 3′v1.1 + miR- | UUGCCCCUUGGGCCUCCCCCCAGCCCCUCCUCCC |
| | | 142-3p binding | CUUCCUGCACCCGUACCCCCUCCAUAAAGUAGGA |
| | | site | AACACUACAGUGGUCUUUGAAUAAAGUCUGAGUG |
| | | | GGCGGC |
| 164 | 5′UTR (DNA) | mSOD2 5UTR | GGGGAGACGTTGCCTTCCCAGGATGCCGCTCCGT |
| | | | TATGCGCCGGGCCGTCCGTGTCGCCGTCCTCCCC |
| | | | TCCGCTGATGGGCGCTGCGGGCAGGGTCACCGCT |
| | | | TCGCTGTGTCCTTGCGGACGCCGGGCGGACGCTG |
| | | | CCTAGCAGACGCGCGCCTGCGAGCGAACGGCCGT |
| | | | GTTCTGAGGAGAGCAGCGGTCGTGTAAACCTCAA |
| | | | TA |
| 165 | 5′UTR (RNA) | mSOD2 5UTR | GGGGAGACGUUGCCUUCCCAGGAUGCCGCUCCGU |
| | | | UAUGCGCCGGGCCGUCCGUGUCGCCGUCCUCCCC |
| | | | UCCGCUGAUGGGCGCUGCGGGCAGGGUCACCGCU |
| | | | UCGCUGUGUCCUUGCGGACGCCGGGCGGACGCUG |
| | | | CCUAGCAGACGCGCGCCUGCGAGCGAACGGCCGU |
| | | | GUUCUGAGGAGAGCAGCGGUCGUGUAAACCUCAA |
| | | | UA |
| 166 | 3′UTR (RNA) | mt-rpS12Δ1 3UTR | UAAUAGUAACAGAAGAAGUGACGGCUGGGGGCAC |
| | | | AGUGGGCUGGGCGCCCCUGCAGAACUAGAACCUU |
| | | | CCGCUCCUGGCUGCCACAGGGUCCUCCGUAGCUG |
| | | | GCCUUUGCGCCUGUAGAGGCAGCCACUCUAGGAU |
| | | | UCAAGUCCUGGCUCCGCCUCUUCCAUCAGGACCA |
| | | | CUA |
| 167 | 3′UTR (RNA) | mSOD2 3UTR | UAAUAGUAAACCUCACUCACGGCCACAUUGAGUG |
| | | altered stop | CCAGGCUCCGGGCUGGUUUAUAGUAGUGUAGAGC |
| | | | AUUGCAGCACUUAGACUGGGGUGCUGUAGUCUUU |
| | | | AUUGUAGUCUUUCCACAUACCUGAUAAUUCUUAG |
| | | | AUAAUUUCUUAUUUUAAUUAAAUCUAUUCUUAGG |
| | | | CU |
| 168 | 3′UTR (RNA) | 3′v1.1m142 | UAAUAGUAAGCUGGAGCCUCGGUGGCCUAGCUUC |
| | | 3′v1.1 + miR- | UUGCCCCUUGGGCCUCCCCCCAGCCCCUCCUCCC |
| | | 142-3p binding | CUUCCUGCACCCGUACCCCCUCCAUAAAGUAGGA |
| | | site | AACACUACAGUGGUCUUUGAAUAAAGUCUGAGUG |
| | | Altered stop | GGCGGC |
| 169 | 3′UTR (DNA) | Sod2_3 | TAATAGTAAACCTCACTCACGGCCACATTGAGTG |
| | | (N3xstop_mSod2_ | CCAGGCTCCGGGCTGGTTTATAGTAGTGTAGAGC |
| | | 3x-142-3p_3) | ATTGCAGCACTTAGACTGGGGTGCTGTAGTCTTT |
| | | | ATTGTAGTCTTTCCACATACCTGATAATTCTTAG |
| | | | ATAATTTCTTATTTTAATTCCATAAAGTAGGAAA |
| | | | CACTACATAAATCTCCATAAAGTAGGAAACACTA |
| | | | CATATTCTTCCATAAAGTAGGAAACACTACATAG |
| | | | GCT |
| 170 | 3′UTR (RNA) | Sod2_3 | UAAUAGUAAACCUCACUCACGGCCACAUUGAGUG |
| | | (N3xstop_mSod2_ | CCAGGCUCCGGGCUGGUUUAUAGUAGUGUAGAGC |
| | | 3x-142-3p_3) | AUUGCAGCACUUAGACUGGGGUGCUGUAGUCUUU |
| | | | AUUGUAGUCUUUCCACAUACCUGAUAAUUCUUAG |
| | | | AUAAUUUCUUAUUUUAAUUCCAUAAAGUAGGAAA |
| | | | CACUACAUAAAUCUCCAUAAAGUAGGAAACACUA |
| | | | CAUAUUCUUCCAUAAAGUAGGAAACACUACAUAG |
| | | | GCU |
| 171 | 3′UTR (DNA) | Sod2_5 | TAATAGTAAACCTCATCCATAAAGTAGGAAACAC |
| | | (N3xstop_mSod2_ | TACACTCACGTCCATAAAGTAGGAAACACTACAG |
| | | 3x-142-3p_5) | CCACATCCATAAAGTAGGAAACACTACATTGAGT |
| | | | GCCAGGCTCCGGGCTGGTTTATAGTAGTGTAGAG |
| | | | CATTGCAGCACTTAGACTGGGGTGCTGTAGTCTT |
| | | | TATTGTAGTCTTTCCACATACCTGATAATTCTTA |
| | | | GATAATTTCTTATTTTAATTAAATCTATTCTTAG |
| | | | GCT |
| 172 | 3′UTR (RNA) | Sod2_5 | UAAUAGUAAACCUCAUCCAUAAAGUAGGAAACAC |
| | | (N3xstop_mSod2_ | UACACUCACGUCCAUAAAGUAGGAAACACUACAG |
| | | 3x-142-3p_5) | CCACAUCCAUAAAGUAGGAAACACUACAUUGAGU |
| | | | GCCAGGCUCCGGGCUGGUUUAUAGUAGUGUAGAG |
| | | | CAUUGCAGCACUUAGACUGGGGUGCUGUAGUCUU |
| | | | UAUUGUAGUCUUUCCACAUACCUGAUAAUUCUUA |
| | | | GAUAAUUUCUUAUUUUAAUUAAAUCUAUUCUUAG |
| | | | GCU |
| 173 | 3′UTR (DNA) | rpS12_3 | TAATAGTAACAGAAGAAGTGACGGCTGGGGGCAC |
| | | (N3xstop_mt- | AGTGGGCTGGGCGCCCCTGCAGAACTAGAACCTT |
| | | rpS12_3x-142- | CCGCTCCTGGCTGCCACAGGGTCCTCCGTAGCTG |
| | | 3p_3) | GCCTTTGCGCCTGTAGAGGCAGCCACTCTAGGAT |
| | | | TCAAGTCCTGGCTCCGCCTTCCATAAAGTAGGAA |
| | | | ACACTACACTTCCATCCATAAAGTAGGAAACACT |
| | | | ACATCAGGATCCATAAAGTAGGAAACACTACACC |
| | | | ACTA |
| 174 | 3′UTR (RNA) | rpS12_3 | UAAUAGUAACAGAAGAAGUGACGGCUGGGGGCAC |
| | | (N3xstop_mt- | AGUGGGCUGGGCGCCCCUGCAGAACUAGAACCUU |
| | | rpS12_3x-142- | CCGCUCCUGGCUGCCACAGGGUCCUCCGUAGCUG |
| | | 3p_3) | GCCUUUGCGCCUGUAGAGGCAGCCACUCUAGGAU |
| | | | UCAAGUCCUGGCUCCGCCUUCCAUAAAGUAGGAA |
| | | | ACACUACACUUCCAUCCAUAAAGUAGGAAACACU |
| | | | ACAUCAGGAUCCAUAAAGUAGGAAACACUACACC |
| | | | ACUA |
| 175 | 3′UTR (DNA) | rpS12_5 | TAATAGTAACAGAAGTCCATAAAGTAGGAAACAC |
| | | (N3xstop_mt- | TACAAAGTGATCCATAAAGTAGGAAACACTACAC |
| | | rpS12_3x-142- | GGCTGTCCATAAAGTAGGAAACACTACAGGGGCA |
| | | 3p_5) | CAGTGGGCTGGGCGCCCCTGCAGAACTAGAACCT |
| | | | TCCGCTCCTGGCTGCCACAGGGTCCTCCGTAGCT |
| | | | GGCCTTTGCGCCTGTAGAGGCAGCCACTCTAGGA |
| | | | TTCAAGTCCTGGCTCCGCCTCTTCCATCAGGACC |
| | | | ACTA |
| 176 | 3′UTR (RNA) | rpS12_5 | UAAUAGUAACAGAAGUCCAUAAAGUAGGAAACAC |
| | | (N3xstop_mt- | UACAAAGUGAUCCAUAAAGUAGGAAACACUACAC |
| | | rpS12_3x-142- | GGCUGUCCAUAAAGUAGGAAACACUACAGGGGCA |
| | | 3p_5) | CAGUGGGCUGGGCGCCCCUGCAGAACUAGAACCU |
| | | | UCCGCUCCUGGCUGCCACAGGGUCCUCCGUAGCU |
| | | | GGCCUUUGCGCCUGUAGAGGCAGCCACUCUAGGA |
| | | | UUCAAGUCCUGGCUCCGCCUCUUCCAUCAGGACC |
| | | | ACUA |
| 177 | miR | mmiR-142 | GACAGUGCAGUCACCCAUAAAGUAGAAAGCACUA |
| | | | CUAACAGCACUGGAGGGUGUAGUGUUUCCUACUU |
| | | | UAUGGAUGAGUGUACUGUG |
| 178 | miR | mmiR-142-3p | UGUAGUGUUUCCUACUUUAUGGA |
| 179 | miR binding | mmiR-142-3p | UCCAUAAAGUAGGAAACACUACA |
| | site | binding site | |
| 180 | miR | mmiR-142-5p | CAUAAAGUAGAAAGCACUACU |
| 181 | miR binding | mmiR-142-5p | AGUAGUGCUUUCUACUUUAUG |
| | site | binding site | |
| 182 | Stop codon | Triple stop 1 | UGAUAAUAG |
| 183 | Stop codon | Triple stop 2 | UAAUAGUAA |
| 184 | Epitope tag | Myc | EQKLISEEDL |
| 185 | Epitope tag | V5 | GKPIPNPLLGLDST |
| 186 | Epitope tag | Hemagglutinin A | YPYDVPDYA |
| | | (HA) | |
| 187 | Epitope tag | 6xHis tag | HHHHHH |
| 188 | Epitope tag | HSV | QPELAPEDPED |
| 189 | Epitope tag | VSV-G | YTDIEMNRLGK |
| 190 | Epitope tag | NE | TKENPRSNQEESYDDNES |
| 191 | Epitope tag | AViTag | GLNDIFEAQKIEWHE |
| 192 | Epitope tag | Calmodulin | KRRWKKNFIAVSAANRFKKISSSGAL |
| 193 | Epitope tag | E tag | GAPVPYPDPLEPR |
| 194 | Epitope tag | S tag | KETAAAKFERQHMDS |
| 195 | Epitope tag | SBP tag | MDEKTTGWRGGHVVEGLAGELEQLRARLEHHPQG |
| | | | QREP |
| 196 | Epitope tag | Softag 1 | SLAELLNAGLGGS |
| 197 | Epitope tag | Softag 3 | TQDPSRVG |
| 198 | Epitope tag | Strep tag | WSHPQFEK |
| 199 | Epitope tag | Ty tag | EVHTNQDPLD |
| 200 | Epitope tag | Xpress tag | DLYDDDDK |

What is claimed is:

1. An mRNA comprising:
a 5′ cap;
a 5′ UTR comprising a structural RNA element comprising a stem-loop;
an ORF encoding a polypeptide; and
a 3′ UTR,
wherein the structural RNA element comprises a sequence of 15-25 linked nucleotides, wherein each nucleotide comprises a nucleobase selected from the group consisting of: adenine, guanine, thymine, uracil, and cytosine, or derivatives or analogs thereof, and wherein the structural RNA element comprises (i) a double-stranded stem of about 4-7 base pairs comprising at least 50% G/C base pairs; (ii) a single-stranded loop of about 3-8 nucleotides; (iii) a deltaG (ΔG) about −10 to −15 kcal/mol, and, optionally, (iv) a nucleotide sequence which differs from SEQ ID NO: 6 or SEQ ID NO: 47 by substitution, deletion or insertion of 1, 2, 3, 4, or 5 nucleotides or a nucleotide sequence at least 80%, 85%, 90%, 95%, 96%, 97%, 98%, or 99% identical to the nucleotide sequence of SEQ ID NO: 6 or the nucleotide sequence of SEQ ID NO: 47.

2. The mRNA of claim 1, wherein the double-stranded stem comprises at least 60%, at least 65%, at least 70%, at least 75%, at least 80%, at least 85%, or at least 90% G/C base pairs.

3. The mRNA of any one of claims 1 or 2, wherein the double-stranded stem comprises 30% or less A/U base pairs.

4. The mRNA of any one of claims 1-3, wherein the single-stranded loop is about 4-7 nucleotides in length.

5. The mRNA of any one of claims 1-4, wherein the structural RNA element comprises at least 50%, at least 60%, at least 65%, at least 70%, at least 75%, at least 80%, at least 85%, or at least 90% G/C bases.

6. The mRNA of claim 5, wherein the structural RNA element comprises at least 60% G/C bases.

7. The mRNA of any one of claims 5 or 6, wherein the structural RNA element comprises 40% or less A/U bases.

8. The mRNA of claim 1, wherein the structural RNA element comprises a nucleotide sequence at least 80%, 85%, 90%, 95%, 96%, 97%, 98%, or 99% identical to the nucleotide sequence of SEQ ID NO: 6.

9. The mRNA of claim 1, wherein the structural RNA element comprises a nucleotide sequence at least 80%, 85%, 90%, 95%, 96%, 97%, 98%, or 99% identical to the nucleotide sequence of SEQ ID NO: 47.

10. The mRNA of any one of the preceding claims, wherein the structural RNA element provides a translational regulatory activity comprising increasing an amount of polypeptide translated from the full open reading frame.

11. An mRNA comprising:
a 5′ cap;
a 5′ UTR comprising a structural RNA element comprising the nucleotide sequence of SEQ ID NO: 6 or SEQ ID NO: 47;
an ORF encoding a polypeptide; and
a 3′ UTR.

12. The mRNA of any one of claims 1-11, wherein the 5′ UTR comprises a Kozak-like sequence upstream of the initiation codon and the structural RNA element is located upstream of the Kozak-like sequence in the 5′ UTR.

13. The mRNA of claim 12, wherein the structural RNA element is located about 45-50, about 40-45, about 35-40, about 30-35, about 25-30, about 20-25, about 15-20, about 10-15, about 6-10 nucleotides, about 1-5 nucleotides, or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide upstream of the Kozak-like sequence in the 5′ UTR, optionally wherein the structural RNA element is located about 40-45, about 10-15, or about 6-10 nucleotides upstream of the Kozak-like sequence in the 5′ UTR.

14. The mRNA of any one of claims 1-13, wherein the structural RNA element is located downstream of the 5′ cap or 5′ end of the mRNA in the 5′ UTR.

15. The mRNA of claim 14, wherein the structural RNA element is located about 45-50, about 40-45, about 35-40, about 30-35, about 25-30, about 20-25, about 15-20, about 10-15, about 5-10 nucleotides, about 1-5 nucleotide(s), or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) downstream of the 5′ cap or 5′ end of the mRNA in the 5′ UTR, optionally wherein the structural RNA element is located about 40-45, about 20-25, or about 5-10 nucleotides downstream of the 5′ cap or 5′ end of the mRNA in the 5′ UTR.

16. The mRNA of any one of the preceding claims, comprising a Kozak-like sequence in the 5′UTR, wherein the 5′UTR comprises a GC-rich RNA element comprising a sequence of about 20-30, about 10-20, about 10-15, about 5-15, or about 3-15 nucleotides, or derivatives or analogs thereof, wherein the sequence is at least about 50% cytosine, and wherein the GC-rich RNA element is located upstream of the Kozak-like in the 5′ UTR, optionally wherein the GC-rich RNA element comprises a sequence of about 3-15, about 15, 14, 13, 12, 11, 10, 9, 8, 7, 6, 5, 4, or 3 nucleotides, or derivatives or analogs thereof, wherein the sequence is about 50%-60% cytosine, about 60%-70% cytosine, or about 70%-80% cytosine, optionally wherein the GC-rich RNA element comprises a sequence of cytosine and guanine, optionally wherein the GC-rich RNA element comprises a sequence of about 3-30 guanine and cytosine nucleotides, or derivatives or analogues thereof, wherein the sequence comprises a repeating GC-motif, wherein the repeating GC-motif is [CCG]ₙ or [GCC]ₙ, wherein n=1 to 10, 1-5, 3, 2 or 1.

17. The mRNA of claim 16, wherein the sequence of the GC-rich RNA element is selected from (i) the sequence of EK1 [CCCGCC] set forth in SEQ ID NO: 3; (ii) the sequence of EK2 [GCCGCC] set forth in SEQ ID NO: 18; and (iii) the sequence of EK3 [CCGCCG] set forth in SEQ ID NO: 19.

18. The mRNA of claim 16, wherein the sequence of the GC-rich RNA element comprises the sequence of V1 [CCCCGGCGCC] set forth in SEQ ID NO: 1 or the sequence of V2 [CCCCGGC] set forth in SEQ ID NO: 2.

19. The mRNA of claim 16, wherein the sequence of the GC-rich RNA element comprises the sequence of CG1 [GCGCCCCGCGGCGCCCCGCG] set forth in SEQ ID NO: 20 or the sequence of CG2 [CCCGCCCGCCCCGCCCCGCC] set forth in SEQ ID NO: 21.

20. The mRNA of any one of claims 16-19, wherein the GC-rich RNA element is located about 20-30, about 15-20, about 10-15, about 5-10, or about 1-5 nucleotides upstream of the Kozak-like sequence in the 5′ UTR, optionally wherein the GC-rich RNA element is located about 5, about 4, about 3, about 2, or 1 nucleotide(s) upstream of the Kozak-like sequence in the 5′ UTR or wherein the GC-rich RNA element is upstream of and immediately adjacent to the Kozak-like sequence in the 5′ UTR, and wherein the Kozak-like sequence comprises the sequence [5‘-GCCACC-’3] set forth in SEQ ID NO: 17 or [5′-GCCGCC-′3] set forth in SEQ ID NO: 48.

21. The mRNA of any one of claims 16-20, wherein the GC-rich RNA element comprises a stable RNA secondary structure located downstream of the initiation codon, optionally wherein the GC-rich RNA element is located about 20-30, about 10-20, about 15-20, about 10-15, about 5-10, or about 1-5 nucleotides downstream of the initiation codon, optionally wherein the stable RNA secondary structure is a hairpin or a stem-loop, optionally wherein the stable RNA secondary structure has a deltaG of about −20 to −30 kcal/mol, about −10 to −20 kcal/mol, or about −5 to −10 kcal/mol.

22. The mRNA of claim 21, wherein the GC-rich RNA element comprising a stable RNA secondary structure selected from (i) the sequence of SL1 [CCGCGGCGCCCCGCGG] as set forth in SEQ ID NO: 24; (ii) the sequence of SL2 [GCGCGCAUAUAGCGCGC] as set forth in SEQ ID NO: 25; (iii) the sequence of SL3 [CAUGGUGGCGGCCCGCCGCCACCAUG] as set forth in SEQ ID NO: 49; (iv) the sequence of SL4 [CAUGGUGGCCCGCCGCCACCAUG] as set forth in SEQ ID NO: 50; and (v) the sequence of SL5 [CAUGGUGCCCGCCGCCACCAUG] as set forth in SEQ ID NO: 51.

23. An mRNA comprising a 5′ cap, a 5′ UTR, an ORF encoding a polypeptide, and a 3′ UTR,
wherein the 5′UTR comprises:
(i) a structural RNA element comprising a stem loop comprising a nucleotide sequence at least 80%, 85%, 90%, 95%, 96%, 97%, 98%, 99% or 100% identical to the nucleotide sequence of SEQ ID NO: 6 or the nucleotide sequence of SEQ ID NO: 47, optionally wherein the structural RNA element has a deltaG (ΔG) of about −20 to −25 kcal/mol, about −15 to −20 kcal/mol, or about −10 to −15 kcal/mol; and
(ii) a GC-rich RNA element comprising a nucleotide sequence selected from the group consisting of: SEQ ID NO: 1, SEQ ID NO: 2, SEQ ID NO: 3, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 49, SEQ ID NO: 50 and SEQ ID NO: 51.

24. The mRNA of claim 23, wherein the GC-rich RNA element comprises a nucleotide sequence selected from the group consisting of: SEQ ID NO: 1, SEQ ID NO: 2 and SEQ ID NO: 23.

25. The mRNA of any one of claims 23 or 24, wherein the mRNA comprises a Kozak-like sequence, and wherein the GC-rich RNA element is located about 1-20 nucleotides upstream of the Kozak-like sequence in the 5′ UTR, optionally wherein the GC-rich RNA element is located about 5, about 4, about 3, about 2, or 1 nucleotide upstream of the Kozak-like sequence in the 5′ UTR or is upstream of and immediately adjacent to the Kozak-like sequence in the 5′ UTR, optionally wherein the Kozak-like sequence comprises the nucleotide sequence [5′-GCCACC-3′] set forth in SEQ ID NO: 17 or the nucleotide sequence [5′-GCCGCC-3′] set forth in SEQ ID NO: 48.

26. The mRNA of any one of claims 23-25, wherein the structural RNA element is upstream of the GC-rich RNA element in the 5′UTR, optionally wherein the structural RNA element is about 1-5, 5-10, 10-20, 20-30, 30-40, or 40-50 nucleotides, or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) upstream of the GC-rich RNA element in the 5′UTR, optionally wherein the structural RNA element is 1-5 nucleotides, 10-20 nucleotides, or 30-40 nucleotides upstream of the GC-rich RNA element in the 5′UTR, optionally wherein the structural RNA element is upstream of and immediately adjacent to the GC-rich RNA element in the 5′UTR.

27. The mRNA of any one of claims 23-26, wherein the structural RNA element is located downstream of the 5′ cap or 5′ end of the mRNA in the 5′ UTR, optionally wherein the structural RNA element is located about 45-50, about 40-45, about 35-40, about 30-35, about 25-30, about 20-25, about 15-20, about 10-15, about 5-10 nucleotides, about 1-5 nucleotide(s), or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) downstream of the 5′ cap or 5′ end of the mRNA in the 5′ UTR, optionally wherein the structural RNA element is located about 40-45 nucleotides, about 20-25 nucleotides, about 5-10 nucleotides downstream of the 5′ cap or 5′ end of the mRNA in the 5′ UTR or wherein the structural RNA element is located downstream of the 5′ cap or 5′ end of the mRNA and immediately adjacent to a transcription start site element in the 5′ UTR.

28. The mRNA of claim 27, wherein the transcription start site element comprises the nucleotide sequence [5′-GGGAAA-3′] set forth in SEQ ID NO: 53 or the nucleotide sequence [5′-AGGAAA-3′] set forth in SEQ ID NO: 54.

29. An mRNA comprising:
a 5′ cap;
a 5′ UTR comprising a structural RNA element comprising a stem-loop, optionally wherein the stem loop comprises a sequence of 15-25 linked nucleotides comprising at least 60% G/C bases, wherein the structural RNA element comprises (i) a double-stranded stem of about 4-7 base pairs; (ii) a single-stranded loop of about 4-7 nucleotides; (iii) a nucleotide sequence which differs from SEQ ID NO: 6 by substitution, deletion or insertion of 1, 2, 3, 4, or 5 nucleotides; and (iv) a delta G (ΔG) of about −10 to −15 kcal/mol, optionally wherein the structural RNA element comprises the nucleotide sequence of SEQ ID NO: 6;
an ORF encoding a polypeptide; and
a 3′ UTR
wherein the structural RNA element comprises a nucleotide sequence at least 80%, 85%, 90%, 95%, 96%, 97%, 98%, 99% or 100% identical to the nucleotide sequence of SEQ ID NO: 6, wherein the 5′ UTR comprises the nucleotide sequence of SEQ ID NO: 4 or SEQ ID NO: 60 comprising a GC-rich RNA element comprising the sequence CCCCGGCGCC (SEQ ID NO: 1), and wherein the structural RNA element is inserted upstream of the GC-rich RNA element in the 5′ UTR.

30. The mRNA of claim 29, wherein the structural RNA element is inserted about 1-5, 5-10, 10-20, 20-30, or 30-40 nucleotides, or about 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) upstream of the GC-rich RNA element in SEQ ID NO: 4 or SEQ ID NO: 60, optionally wherein the structural RNA element is inserted 1-5 nucleotides, 10-20 nucleotides or 30-40 nucleotides upstream of the GC-rich RNA element in SEQ ID NO: 4 or SEQ ID NO: 60, or wherein the structural RNA element is inserted upstream of and immediately adjacent to the GC-rich RNA element in SEQ ID NO: 4 or SEQ ID NO: 60.

31. An mRNA comprising:
a 5′ cap;
a 5′ UTR;
an ORF encoding a polypeptide; and
a 3′ UTR
wherein the 5′ UTR comprises a nucleotide sequence selected from the group consisting of:
(i) the nucleotide sequence of SEQ ID NO: 116;
(ii) the nucleotide sequence of SEQ ID NO: 120;
(iii) the nucleotide sequence of SEQ ID NO: 124;
(iv) the nucleotide sequence of SEQ ID NO: 128; and
(v) the nucleotide sequence of SEQ ID NO: 41.

32. An mRNA comprising:
(i) a 5′ cap;
(ii) a 5′UTR;
(iii) an ORF encoding a polypeptide; and
(iv) a 3′UTR,
wherein the 3′UTR comprises a nucleotide sequence of a 3′UTR of a nuclear-encoded mitochondrially derived protein (NEMP), optionally wherein binding of the 3′UTR to one or more RNA-binding proteins promotes the stabilization, localization, and/or translation of the mRNA, optionally wherein the NEMP is selected from the group consisting of: human OXAL1, human MRPS12, and mouse Sod2.

33. The mRNA of claim 32, wherein the nucleotide sequence of the 3′UTR is at least 70%, at least 75%, at least 80%, at least 85%, at least 90%, at least 95%, at least 96%, at least 97%, at least 98%, at least 99%, or 100% identical to the nucleotide sequence of the NEMP 3′UTR, or wherein the 3′UTR differs from the nucleotide sequence of the NEMP 3′UTR by 1-5, 5-10, 10-15, 15-20, 20-25, 25-30, 30-35, 35-40, 40-45, 45-50 or about 50 or more nucleotides, optionally wherein the 3′UTR is about 50-100 nucleotides, about 100-200 nucleotides, about 200-300 nucleotides, about 300-400 nucleotides, about 400-500 nucleotides, about 500-600, about 600-700 nucleotides, about 700-800 nucleotides, about 800-900 nucleotides, about 900-1000 nucleotides, about 1000-1100 nucleotides, about 1100-1200 nucleotides, about 1200-1300 nucleotides, about 1300-1400 nucleotides, or about 1400-1500 nucleotides in length.

34. The mRNA of any one of claims 32 or 33, wherein the 3′UTR comprises a nucleotide sequence at least 70%, at least 75%, at least 80%, at least 85%, at least 90%, at least 95%, at least 96%, at least 97%, at least 98%, at least 99% identical or 100% identical to a nucleotide sequence selected from the group consisting of: SEQ ID NO: 72; SEQ ID NO: 74; SEQ ID NO: 76; SEQ ID NO: 78; SEQ ID NO: 166; and SEQ ID NO: 167, or wherein the 3′UTR differs from the NEMP 3′UTR by about 1-5, 5-10, 10-15, 15-20, 20-25, 25-30, 30-35, 35-40, 40-45, 45-50 or about 50-100 nucleotides, wherein the NEMP 3′UTR comprises a nucleotide sequence selected from the group consisting of: SEQ ID NO: 72; SEQ ID NO: 74; SEQ ID NO: 76; SEQ ID NO: 78; SEQ ID NO: 166; and SEQ ID NO: 167.

35. The mRNA of any one of claims 32-34, wherein the 3′UTR comprises one or more microRNA (miRNA) binding sites, optionally wherein the 3′UTR comprises 1, 2, 3, 4, 5, 6, 7, 8, 9, or 10 miRNA binding site(s), optionally wherein the miRNA binding site is targeted by miR-142-3p or miR-142-5p, optionally wherein the miRNA binding site comprises a nucleotide sequence at least 80%, at least 85%, at least 90%, at least 95%, at least 96%, at least 97%, at least 98%, at least 99%, or 100% identical to the nucleotide sequence of SEQ ID NO: 179 or SEQ ID NO: 181.

36. The mRNA of claim 35, wherein the 3′UTR comprises one or more stop codons at the 5′end of the 3′UTR, and wherein the 3′UTR comprises 1, 2, 3, or 4 miRNA binding sites located proximal to the one or more stop codons, optionally wherein the miRNA binding site(s) are located downstream of and immediately adjacent to the one or more stop codons at the 5′end of the 3′UTR, optionally wherein the miRNA binding sites are located about 45-50, about 40-45, about 35-40, about 30-35, about 25-30, about 20-25, about 15-20, about 10-15, about 6-10 nucleotides, about 1-5 nucleotide(s), or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) downstream of the one or more stop codons at the 5′end of the 3′UTR, optionally wherein the miRNA binding sites are located about 10, about 9, about 8, about 7, about 6, about 5, about 4, about 3, about 2, or about 1 nucleotide(s) downstream of the one or more stop codons at the 5′end of the 3′UTR.

37. The mRNA of claim 35, wherein the 3′UTR comprises 1, 2, 3, or 4 miRNA binding sites located proximal to the 3′end of the 3′UTR, optionally wherein the miRNA binding site(s) are located upstream of and immediately adjacent to the 3′end of the 3′UTR, optionally wherein the miRNA binding site(s) are located about 1-5, about 6-10, about 10-15, about 15-20, about 20-25, about 25-30, about 30-35, about 35-40, about 40-45, or about 45-50 nucleotide(s) or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotide(s) upstream of the 3′end of the 3′UTR, optionally wherein the miRNA binding site(s) are located about 1, about 2, about 3, about 4, or about 5, about 6, about 7, about 8, about 9 or about 10 nucleotide(s) upstream of the 3′end of the 3′UTR.

38. The mRNA of any one of claim 35-37, wherein the 3′UTR comprises 1, 2, 3, 4, 5, 6, 7, 8, 9, or 10 miRNA binding sites, wherein an upstream miRNA binding site is located directly adjacent to one or more downstream miRNA binding site(s), optionally wherein the 3′UTR comprises 1, 2, 3, 4, 5, 6, 7, 8, 9, or 10 miRNA binding sites, wherein an upstream miRNA binding site is separated from a downstream miRNA binding site by about 1-5, about 1-10, about 5-10, about 5-15, about 10-20, about 15-20, about 15-30, or about 20-30 nucleotide(s) or about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotide(s), optionally wherein the 3′UTR comprises 1, 2, 3, 4, 5, 6, 7, 8, 9, or 10 miRNA binding sites, wherein an upstream miRNA binding site is separated from a downstream miRNA binding site by about 1, about 2, about 3, about 4, about 5, about 6, about 7, about 8, about 9 or about 10 nucleotide(s).

39. The mRNA of any one of claim 32, wherein the 3′ UTR comprises
(i) a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 78, wherein the 3′UTR comprises 1, 2, 3, or 4 miR-142-3p binding sites, and wherein the miR-142-3p binding site comprises the nucleotide sequence of SEQ ID NO: 179; or
(ii) a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 76, wherein the 3′UTR comprises 1, 2, 3, or 4 miR-142-3p binding sites, and wherein the miR-142-3p binding site comprises the nucleotide sequence of SEQ ID NO: 179.

40. The mRNA of claim 39, wherein the 1, 2, 3, or 4 miR-142-3p binding sites are located proximal to the 3′end or the 3′UTR, optionally wherein the 3′UTR comprises one or more stop codons at the 5′end and wherein the 1, 2, 3, or 4 miR-142-3p binding sites are located proximal to the one or more stop codons.

41. The mRNA of claim 39, wherein the 3′UTR comprises a nucleotide sequence selected from SEQ ID NO: 170, 172, and 174 and 176.

42. An mRNA comprising:
a 5′ cap;
a 5′ UTR comprising a structural RNA element comprising a stem-loop, wherein the structural RNA element comprises a nucleotide sequence at least 80%, 85%, 90%, 95%, 96%, 97%, 98%, 99% or 100% identical to the nucleotide sequence of SEQ ID NO: 6 or wherein the structural RNA element comprises a sequence of 15-25 linked nucleotides comprising at least 60% G/C bases, wherein the structural RNA element comprises (i) a double-stranded stem of about 4-7 base pairs; (ii) a single-stranded loop of about 4-7 nucleotides; (iii) a nucleotide sequence which differs from SEQ ID NO: 6 by substitution, deletion or insertion of 1, 2, 3, 4, or 5 nucleotides; and (iv) a delta G (ΔG) of about −10 to −15 kcal/mol;
an ORF encoding a polypeptide; and
a 3′ UTR, wherein the 3′UTR comprises a nucleotide sequence at least 70%, at least 75%, at least 80%, at least 85%, at least 90%, at least 95%, at least 96%, at least 97%, at least 98%, at least 99% identical or 100% identical to a nucleotide sequence of SEQ ID NO: 76; SEQ ID NO: 78; SEQ ID NO: 166; or SEQ ID NO: 167,
wherein the 5′ UTR comprises the nucleotide sequence of SEQ ID NO: 4 or SEQ ID NO: 60 comprising a GC-rich RNA element comprising the sequence CCCCGGCGCC (SEQ ID NO: 1), and wherein the structural RNA element is inserted upstream of the GC-rich RNA element in the 5′ UTR.

43. The mRNA of claim 42, wherein the structural RNA element is inserted about 1-5, 5-10, 10-20, 20-30, or 30-40 nucleotides, or about 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) upstream of the GC-rich RNA element in SEQ ID NO: 4 or SEQ ID NO: 60, optionally wherein the structural RNA element is inserted 1-5 nucleotides, 10-20 nucleotides, or 30-40 nucleotides upstream of the GC-rich RNA element in SEQ ID NO: 4 or SEQ ID NO: 60, or wherein the structural RNA element is inserted upstream of and immediately adjacent to the GC-rich RNA element in SEQ ID NO: 4 or SEQ ID NO: 60.

44. An mRNA comprising:
a 5′ cap;
a 5′ UTR, wherein the 5′ UTR comprises a nucleotide sequence selected from the group consisting of:
(i) the nucleotide sequence of SEQ ID NO: 116;
(ii) the nucleotide sequence of SEQ ID NO: 120;
(iii) the nucleotide sequence of SEQ ID NO: 124;
(iv) the nucleotide sequence of SEQ ID NO: 41; and
(v) the nucleotide sequence of SEQ ID NO: 128;
an ORF encoding a polypeptide; and
a 3′ UTR, wherein the 3′UTR comprises a nucleotide sequence at least 70%, at least 75%, at least 80%, at least 85%, at least 90%, at least 95%, at least 96%, at least 97%, at least 98%, at least 99% identical or 100% identical to a nucleotide sequence of SEQ ID NO: 76; SEQ ID NO: 78; SEQ ID NO: 166; and SEQ ID NO: 167.

45. The mRNA of claim 44, wherein the 3′UTR comprises one or more microRNA (miRNA) binding sites, optionally wherein the 3′UTR comprises 1, 2, 3, 4, 5, 6, 7, 8, 9, or 10 miRNA binding site(s), optionally wherein the miRNA binding site is targeted by miR-142-3p or miR-142-5p.

46. The mRNA of claim 45, wherein the miRNA binding site comprises a nucleotide sequence at least 80%, at least 85%, at least 90%, at least 95%, at least 96%, at least 97%, at least 98%, at least 99%, or 100% identical to the nucleotide sequence of SEQ ID NO: 179 or SEQ ID NO: 181.

47. The mRNA of any one of claims 44-46, wherein the 3′UTR comprises one or more microRNA (miRNA) binding sites, optionally wherein the 3′UTR comprises 1, 2, 3, 4, 5, 6, 7, 8, 9, or 10 miRNA binding site(s), optionally wherein the miRNA binding site is targeted by miR-142-3p or miR-142-5p, optionally wherein the miRNA binding site comprises a nucleotide sequence at least 80%, at least 85%, at least 90%, at least 95%, at least 96%, at least 97%, at least 98%, at least 99%, or 100% identical to the nucleotide sequence of SEQ ID NO: 179 or SEQ ID NO: 181.

48. The mRNA of claim 47, wherein the 3′UTR comprises one or more stop codons at the 5′end of the 3′UTR, and wherein the 3′UTR comprises 1, 2, 3, or 4 miRNA binding sites located proximal to the one or more stop codons, optionally wherein the miRNA binding site(s) are located downstream of and immediately adjacent to the one or more stop codons at the 5′end of the 3′UTR, optionally wherein the miRNA binding sites are located about 45-50, about 40-45, about 35-40, about 30-35, about 25-30, about 20-25, about 15-20, about 10-15, about 6-10 nucleotides, about 1-5 nucleotide(s), or about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 nucleotide(s) downstream of the one or more stop codons at the 5′end of the 3′UTR, optionally wherein the miRNA binding sites are located about 10, about 9, about 8, about 7, about 6, about 5, about 4, about 3, about 2, or about 1 nucleotide(s) downstream of the one or more stop codons at the 5′end of the 3′UTR.

49. The mRNA of claim 47, wherein the 3′UTR comprises 1, 2, 3, or 4 miRNA binding sites located proximal to the 3′end of the 3′UTR, optionally wherein the miRNA binding site(s) are located upstream of and immediately adjacent to the 3′end of the 3′UTR, optionally wherein the miRNA binding site(s) are located about 1-5, about 6-10, about 10-15, about 15-20, about 20-25, about 25-30, about 30-35, about 35-40, about 40-45, or about 45-50 nucleotide(s) or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotide(s) upstream of the 3′end of the 3′UTR, optionally wherein the miRNA binding site(s) are located about 1, about 2, about 3, about 4, or about 5, about 6, about 7, about 8, about 9 or about 10 nucleotide(s) upstream of the 3′end of the 3′UTR.

50. The mRNA of any one of claim 48 or 49, wherein the 3′UTR comprises 1, 2, 3, 4, 5, 6, 7, 8, 9, or 10 miRNA binding sites, wherein an upstream miRNA binding site is located directly adjacent to one or more downstream miRNA binding site(s), optionally wherein the 3′UTR comprises 1, 2, 3, 4, 5, 6, 7, 8, 9, or 10 miRNA binding sites, wherein an upstream miRNA binding site is separated from a downstream miRNA binding site by about 1-5, about 1-10, about 5-10, about 5-15, about 10-20, about 15-20, about 15-30, or about 20-30 nucleotide(s) or about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotide(s), optionally wherein the 3′UTR comprises 1, 2, 3, 4, 5, 6, 7, 8, 9, or 10 miRNA binding sites, wherein an upstream miRNA binding site is separated from a downstream miRNA binding site by about 1, about 2, about 3, about 4, about 5, about 6, about 7, about 8, about 9 or about 10 nucleotide(s).

51. The mRNA of claim 44, wherein the 3′ UTR comprises
(i) a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 78, wherein the 3′UTR comprises 1, 2, 3, or 4 miR-142-3p binding sites, and wherein the miR-142-3p binding site comprises the nucleotide sequence of SEQ ID NO: 179; or
(ii) a nucleotide sequence at least 70%, at least 75%, at least 80%, at least 85%, at least 90%, at least 95%, at least 96%, at least 97%, at least 98%, at least 99%, or 100% identical to the nucleotide sequence of SEQ ID NO: 76, wherein the 3′UTR comprises 1, 2, 3, or 4 miR-142-3p binding sites, and wherein the miR-142-3p binding site comprises the nucleotide sequence of SEQ ID NO: 179.

52. The mRNA of claim 51, wherein the 1, 2, 3, or 4 miR-142-3p binding sites are located proximal to the 3′end or the 3′UTR, optionally wherein the 3′UTR comprises one or more stop codons at the 5′end and wherein the 1, 2, 3, or 4 miR-142-3p binding sites are located proximal to the one or more stop codons.

53. The mRNA of claim 51, wherein the 3′UTR comprises a nucleotide sequence selected from SEQ ID NO: 170, 172, and 174 and 176.

54. An mRNA comprising
a 5′ cap;
a 5′ UTR, wherein the 5′ UTR comprises a nucleotide sequence selected from the group consisting of:
(i) the nucleotide sequence of SEQ ID NO: 116;
(ii) the nucleotide sequence of SEQ ID NO: 120;
(iii) the nucleotide sequence of SEQ ID NO: 124;
(iv) the nucleotide sequence of SEQ ID NO: 41; and
(v) the nucleotide sequence of SEQ ID NO: 128;
an ORF encoding a polypeptide; and
a 3′ UTR, wherein the 3′UTR comprises a nucleotide sequence selected from the group consisting of:
(i) the nucleotide sequence of SEQ ID NO: 170,
(ii) the nucleotide sequence of SEQ ID NO: 172,
(iii) the nucleotide sequence of SEQ ID NO: 174; and
(iv) the nucleotide sequence of SEQ ID NO: 176.

55. An mRNA comprising
a 5′ cap;
a 5′ UTR;
an ORF encoding a polypeptide; and
a 3′ UTR,
wherein the 5′ UTR and 3′ UTR are selected from the group consisting of:
(i) the nucleotide sequence of SEQ ID NO: 120 and the nucleotide sequence of SEQ ID NO: 170;
(ii) the nucleotide sequence of SEQ ID NO: 120 and the nucleotide sequence of SEQ ID NO: 172;
(iii) the nucleotide sequence of SEQ ID NO: 120 and the nucleotide sequence of SEQ ID NO: 174;
(iv) the nucleotide sequence of SEQ ID NO: 120 and the nucleotide sequence of SEQ ID NO: 176;
(v) the nucleotide sequence of SEQ ID NO: 41 and the nucleotide sequence of SEQ ID NO: 170;
(vi) the nucleotide sequence of SEQ ID NO: 41 and the nucleotide sequence of SEQ ID NO: 172;
(vii) the nucleotide sequence of SEQ ID NO: 41 and the nucleotide sequence of SEQ ID NO: 174;
(viii) the nucleotide sequence of SEQ ID NO: 41 and the nucleotide sequence of SEQ ID NO: 176;
(ix) the nucleotide sequence of SEQ ID NO: 128 and the nucleotide sequence of SEQ ID NO: 170;
(x) the nucleotide sequence of SEQ ID NO: 128 and the nucleotide sequence of SEQ ID NO: 172;
(xi) the nucleotide sequence of SEQ ID NO: 128 and the nucleotide sequence of SEQ ID NO: 174; and
(xii) the nucleotide sequence of SEQ ID NO: 128 and the nucleotide sequence of SEQ ID NO: 176.

56. The mRNA of any one of the preceding claims, wherein the mRNA comprises at least one chemically modified nucleoside, and/or wherein the mRNA comprises at least one endonuclease sensitive sequence motif, wherein the endonuclease sensitive sequence motif comprises the nucleotide sequence WGA, wherein W=adenine (A) or uracil (U), and wherein the at least one endonuclease sensitive sequence motif is altered by substitution or deletion, thereby increasing mRNA stability, increasing mRNA half-life, and/or decreasing resistance or susceptibility of the mRNA to endonuclease activity, optionally wherein the at least one chemically modified nucleoside is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine, optionally wherein at least about 10%, at least about 20%, at least about 30%, at least about 40%, at least about 50%, at least about 60%, at least about 70%, at least about 80%, at least about 90%, at least about 95%, at least about 99%, or about 100% of the nucleosides comprising the mRNA comprise the at least one chemically modified nucleoside, optionally wherein the at least one chemically modified nucleoside is N1-methylpseudouridine, and wherein at least 80%, at least 85%, at least 90%, at least 95%, at least 99%, or 100% of the uracil nucleotides are N1-methylpseudouridine, optionally wherein the mRNA is fully modified with N1-methylpseudouridine.

57. The mRNA of claim 56, wherein the at least one modified nucleoside is 5-methoxyuridine, optionally wherein at least 95% of uracil nucleotides comprising the ORF comprise 5-methoxyuridine, and wherein the uracil content in the ORF is between about 100% and about 150% of the theoretical minimum, optionally wherein the mRNA is fully modified with 5-methoxyuridine.

58. The mRNA of any one of the preceding claims comprising a poly A tail.

59. The mRNA of any one of the preceding claims, wherein the mRNA comprises a 5′Cap 1 structure.

60. The mRNA of any one of the preceding claims, wherein an expression level and/or an activity of the polypeptide translated from the mRNA is increased relative to an mRNA that does not comprise the 5′ UTR, 3′ UTR, or a combination thereof.

61. A pharmaceutical composition comprising the mRNA of any one of the preceding claims and a pharmaceutically acceptable carrier.

62. A lipid nanoparticle comprising the mRNA of any one of claims 1-60.

63. The lipid nanoparticle of claim 62, wherein the lipid nanoparticle comprises an ionizable lipid, a sterol, a phospholipid, and a polyethylene glycol lipid.

64. A pharmaceutical composition comprising the lipid nanoparticle of claims 62 or 63, and a pharmaceutically acceptable carrier.

65. The mRNA of any one of claims 1-60, the pharmaceutical composition of claim 61 or 64, or the lipid nanoparticle of claim 62 or 63, for use in treating or delaying progression of a disease or disorder in a subject in need thereof.

66. Use of the mRNA of any one of claims 1-60, the pharmaceutical composition of claim 61 or 64, or the lipid nanoparticle of claim 62 or 63, in the manufacture of a medicament for treating or delaying progression of a disease or disorder in a subject in need thereof.

67. A kit comprising a container comprising the mRNA of any one of claims 1-60, the pharmaceutical composition of claim 61 or 64, or the lipid nanoparticle of claim 62 or 63, and a package insert comprising instructions for administration of the mRNA, the pharmaceutical composition of lipid nanoparticle, for treating or delaying progression of a disease or disorder in a subject.

68. A method of treating or delaying progression of a disease or disorder in a subject in need thereof, the method comprising administering the mRNA according to any one of claims 1-60, the pharmaceutical composition claim 61 or 64, or the lipid nanoparticle according to claim 62 or 63, thereby treating or delaying progression of the disease or disorder in the subject.


SEQUENCE LISTING (244 sequences)

SEQ ID NO: 1 - RNA, 10 nt, Artificial Sequence
  Synthetic RNA Element, V1
ccccggcgcc

SEQ ID NO: 2 - RNA, 7 nt, Artificial Sequence
  Synthetic RNA Element, V2
ccccggc

SEQ ID NO: 3 - RNA, 6 nt, Artificial Sequence
  Synthetic RNA Element, EK1
cccgcc

SEQ ID NO: 4 - RNA, 57 nt, Artificial Sequence
  Synthetic 5′UTR (RNA), 5′v1.1 (V1)
gggaaauaagagagaaaagaagaguaagaagaaauauaagaccccggcgccgccacc

SEQ ID NO: 5 - DNA, 20 bp, Artificial Sequence
  Synthetic RNA Element (DNA), RNAse P stem loop
tctccctgagcttcagggag

SEQ ID NO: 6 - RNA, 20 nt, Artificial Sequence
  Synthetic RNA Element (RNA), RNAse P stem loop
ucucccugagcuucagggag

SEQ ID NO: 7 - DNA, 20 bp, Artificial Sequence
  Synthetic RNA Element (DNA), MRP stem loop
agaagcgtatcccgctgagc

SEQ ID NO: 8 - RNA, 35 nt, Artificial Sequence
  Synthetic 5′UTR (RNA), 5′v1.0 Core (minus leader, Kozak)
uaagagagaaaagaagaguaagaagaaauauaaga

SEQ ID NO: 9 - DNA, 57 bp, Artificial Sequence
  Synthetic 5′UTR (DNA), 5′v1.1 (V1)
gggaaataagagagaaaagaagagtaagaagaaatataagaccccggcgccgccacc

SEQ ID NO: 10 - DNA, 54 bp, Artificial Sequence
  Synthetic 5′UTR (DNA), V2-5′UTR (V2)
gggaaataagagagaaaagaagagtaagaagaaatataagaccccggcgccacc

SEQ ID NO: 11 - DNA, 67 bp, Artificial Sequence
  Synthetic 5′UTR (DNA), CG1-5′UTR
gggaaataagagagaaaagaagagtaagaagaaatataagagcgccccgcggcgccccgcggccacc

SEQ ID NO: 12 - DNA, 67 bp, Artificial Sequence
  Synthetic 5′UTR (DNA), CG2-5′UTR
gggaaataagagagaaaagaagagtaagaagaaatataagacccgcccgccccgccccgccgccacc

SEQ ID NO: 13 - RNA, 15 nt, Artificial Sequence
  Synthetic 5′UTR, KT1-UTR
gggcccgccgccaac

SEQ ID NO: 14 - RNA, 15 nt, Artificial Sequence
  Synthetic 5′UTR, KT2-UTR
gggcccgccgccacc

SEQ ID NO: 15 - RNA, 15 nt, Artificial Sequence
  Synthetic 5′UTR, KT3-UTR
gggcccgccgccgac

SEQ ID NO: 16 - RNA, 15 nt, Artificial Sequence
  Synthetic 5′UTR, KT4-UTR
gggcccgccgccgcc

SEQ ID NO: 17 - RNA, 6 nt, Artificial Sequence
  Synthetic RNA Element, Kozak - 1
gccacc

SEQ ID NO: 18 - RNA, 6 nt, Artificial Sequence
  Synthetic RNA Element, EK2
gccgcc

SEQ ID NO: 19 - RNA, 6 nt, Artificial Sequence
  Synthetic RNA Element, EK3
ccgccg

SEQ ID NO: 20 - RNA, 20 nt, Artificial Sequence
  Synthetic RNA Element, CG1
gcgccccgcggcgccccgcg

SEQ ID NO: 21 - RNA, 20 nt, Artificial Sequence
  Synthetic RNA Element, CG2
cccgcccgccccgccccgcc

SEQ ID NO: 22 - RNA, 30 nt, Artificial Sequence
  Synthetic RNA Element
  misc_feature (1)..(30): This region contains n repeats of “CCG” wherein n = 1 - 10; some positions may or may not be present
ccgccgccgccgccgccgccgccgccgccg

SEQ ID NO: 23 - RNA, 30 nt, Artificial Sequence
  Synthetic RNA Element
  misc_feature (1)..(30): This region contains n repeats of “GCC” wherein n = 1 - 10; some positions may or may not be present
gccgccgccgccgccgccgccgccgccgcc

SEQ ID NO: 24 - RNA, 16 nt, Artificial Sequence
  Synthetic RNA Element, SL1
ccgcggcgccccgcgg

SEQ ID NO: 25 - RNA, 17 nt, Artificial Sequence
  Synthetic RNA Element, SL2
gcgcgcauauagcgcgc

SEQ ID NO: 26 - DNA, 26 bp, Artificial Sequence
  Synthetic RNA Element (DNA), SL3
catggtggcggcccgccgccaccatg

SEQ ID NO: 27 - DNA, 23 bp, Artificial Sequence
  Synthetic RNA Element (DNA), SL4
catggtggcccgccgccaccatg

SEQ ID NO: 28 - DNA, 22 bp, Artificial Sequence
  Synthetic RNA Element (DNA), SL5
catggtgcccgccgccaccatg

SEQ ID NO: 29 - RNA, 12 nt, Artificial Sequence
  Synthetic RNA Element, CR2
cccccccaaccc

SEQ ID NO: 30 - RNA, 12 nt, Artificial Sequence
  Synthetic RNA Element, CR1
ccccccccaacc

SEQ ID NO: 31 - RNA, 12 nt, Artificial Sequence
  Synthetic RNA Element, CR3
ccccccaccccc

SEQ ID NO: 32 - RNA, 12 nt, Artificial Sequence
  Synthetic RNA Element, CR4
ccccccuaagcc

SEQ ID NO: 33 - RNA, 10 nt, Artificial Sequence
  Synthetic RNA Element, CR5
ccccacaacc

SEQ ID NO: 34 - RNA, 11 nt, Artificial Sequence
  Synthetic RNA Element, CR6
cccccacaacc

SEQ ID NO: 35 - RNA, 76 nt, Artificial Sequence
  Synthetic 5′UTR (RNA), combo1_V1.1 (CR3, V1)
gggaaaccccccacccccggggaaauaagagagaaaagaagaguaagaagaaauauaagaccccggcgccgccacc

SEQ ID NO: 36 - RNA, 75 nt, Artificial Sequence
  Synthetic 5′UTR (RNA), combo2_V1.1 (CR5, V1)
gggaaauccccacaaccggggaaauaagagagaaaagaagaguaagaagaaauauaagaccccggcgccgccacc

SEQ ID NO: 37 - RNA, 205 nt, Artificial Sequence
  Synthetic 5′UTR (RNA), combo1_S065 (CR3)
gggaaaccccccacccccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuaaagcagagaa

SEQ ID NO: 38 - RNA, 204 nt, Artificial Sequence
  Synthetic 5′UTR (RNA), combo2_S065 (CR5)
gggaaauccccacaaccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuaaagcagagaa

SEQ ID NO: 39 - RNA, 192 nt, Artificial Sequence
  Synthetic 5′UTR (RNA), combo3_S065 (S065 core extended Kozak)
gggagaccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuacgccgccacc

SEQ ID NO: 40 - RNA, 205 nt, Artificial Sequence
  Synthetic 5′UTR (RNA), combo4_S065 (CR3)
gggaaaccccccacccccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuacgccgccacc

SEQ ID NO: 41 - RNA, 204 nt, Artificial Sequence
  Synthetic 5′UTR (RNA), F153, combo5_S065 (CR5)
gggaaauccccacaaccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuacgccgccacc

SEQ ID NO: 42 - RNA, 192 nt, Artificial Sequence
  Synthetic 5′UTR (RNA), S065
gggagaccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuaaagcagagaa

SEQ ID NO: 43 - RNA, 21 nt, Artificial Sequence
  Synthetic 5′UTR, GCC3-ExtKozak
gggaaagccgccgccgccacc

SEQ ID NO: 44 - RNA, 33 nt, Artificial Sequence
  Synthetic 5′UTR (RNA), CrichCR4 + GCC3-ExtKozak (CR4, GCC3)
gggaaaccccccuaagccgccgccgccgccacc

SEQ ID NO: 45 - RNA, 47 nt, Artificial Sequence
  Synthetic 5′UTR (RNA), 5′v1.0
gggaaauaagagagaaaagaagaguaagaagaaauauaagagccacc

SEQ ID NO: 46 - RNA, 176 nt, Artificial Sequence
  Synthetic 5′UTR (RNA), S065 core
ccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccua

SEQ ID NO: 47 - RNA, 20 nt, Artificial Sequence
  Synthetic RNA Element (RNA), MRP stem loop (RNA)
agaagcguaucccgcugagc

SEQ ID NO: 48 - RNA, 6 nt, Artificial Sequence
  Synthetic RNA Element, Kozak - 2
gccgcc

SEQ ID NO: 49 - RNA, 26 nt, Artificial Sequence
  Synthetic RNA Element (RNA), SL3
caugguggcggcccgccgccaccaug

SEQ ID NO: 50 - RNA, 23 nt, Artificial Sequence
  Synthetic RNA Element (RNA), SL4
caugguggcccgccgccaccaug

SEQ ID NO: 51 - RNA, 22 nt, Artificial Sequence
  Synthetic RNA Element (RNA), SL5
cauggugcccgccgccaccaug

SEQ ID NO: 52 - RNA, 4 nt, Artificial Sequence
  Synthetic RNA Element, F153 GC
cgcc

SEQ ID NO: 53 - RNA, 6 nt, Artificial Sequence
  Synthetic 5′UTR leader, G-start
gggaaa

SEQ ID NO: 54 - RNA, 6 nt, Artificial Sequence
  Synthetic 5′UTR leader, A-start
aggaaa

SEQ ID NO: 55 - DNA, 35 bp, Artificial Sequence
  Synthetic 5′UTR (DNA), 5′v1.0 Core
taagagagaaaagaagagtaagaagaaatataaga

SEQ ID NO: 56 - RNA, 41 nt, Artificial Sequence
  Synthetic 5′UTR (RNA), 5′v1.0 Minus Kozak
gggaaauaagagagaaaagaagaguaagaagaaauauaaga

SEQ ID NO: 57 - RNA, 41 nt, Artificial Sequence
  Synthetic 5′UTR (RNA), 5′v1.0 Minus Leader
uaagagagaaaagaagaguaagaagaaauauaagagccacc

SEQ ID NO: 58 - DNA, 47 bp, Artificial Sequence
  Synthetic 5′UTR (DNA), 5′v1.0
gggaaataagagagaaaagaagagtaagaagaaatataagagccacc

SEQ ID NO: 59 - RNA, 47 nt, Artificial Sequence
  Synthetic 5′UTR (RNA), 5′v1.0 A-start
aggaaauaagagagaaaagaagaguaagaagaaauauaagagccacc

SEQ ID NO: 60 - RNA, 57 nt, Artificial Sequence
  Synthetic 5′UTR (RNA), 5′v1.1 (V1) A-start
aggaaauaagagagaaaagaagaguaagaagaaauauaagaccccggcgccgccacc

SEQ ID NO: 61 - RNA, 51 nt, Artificial Sequence
  Synthetic 5′UTR (RNA), 5′v1.1 (V1) minus leader
uaagagagaaaagaagaguaagaagaaauauaagaccccggcgccgccacc

SEQ ID NO: 62 - RNA, 54 nt, Artificial Sequence
  Synthetic 5′UTR (RNA), V2-5′UTR (V2)
gggaaauaagagagaaaagaagaguaagaagaaauauaagaccccggcgccacc

SEQ ID NO: 63 - RNA, 54 nt, Artificial Sequence
  Synthetic 5′UTR (RNA), V2-5′UTR A-start
aggaaauaagagagaaaagaagaguaagaagaaauauaagaccccggcgccacc

SEQ ID NO: 64 - RNA, 48 nt, Artificial Sequence
  Synthetic 5′UTR (RNA), V2-5′UTR minus leader
uaagagagaaaagaagaguaagaagaaauauaagaccccggcgccacc

SEQ ID NO: 65 - RNA, 67 nt, Artificial Sequence
  Synthetic 5′UTR (RNA), CG1-5′UTR
gggaaauaagagagaaaagaagaguaagaagaaauauaagagcgccccgcggcgccccgcggccacc

SEQ ID NO: 66 - RNA, 67 nt, Artificial Sequence
  Synthetic 5′UTR (RNA), CG1-5′UTR A-start
aggaaauaagagagaaaagaagaguaagaagaaauauaagagcgccccgcggcgccccgcggccacc

SEQ ID NO: 67 - RNA, 61 nt, Artificial Sequence
  Synthetic 5′UTR (RNA), CG1-5′UTR Minus leader
uaagagagaaaagaagaguaagaagaaauauaagagcgccccgcggcgccccgcggccacc

SEQ ID NO: 68 - RNA, 67 nt, Artificial Sequence
  Synthetic 5′UTR (RNA), CG2-5′UTR
gggaaauaagagagaaaagaagaguaagaagaaauauaagacccgcccgccccgccccgccgccacc

SEQ ID NO: 69 - DNA, 119 bp, Artificial Sequence
  Synthetic 3′UTR (DNA), 3′v1.1
tgataataggctggagcctcggtggcctagcttcttgccccttgggcctccccccagcccctcctccccttcctgcacccgtacccccgtggtctttgaataaagtctgagtgggcggc

SEQ ID NO: 70 - RNA, 119 nt, Artificial Sequence
  Synthetic 3′UTR (RNA), 3′v1.1
ugauaauaggcuggagccucgguggccuagcuucuugccccuugggccuccccccagccccuccuccccuuccugcacccguacccccguggucuuugaauaaagucugagugggcggc

SEQ ID NO: 71 - DNA, 233 bp, Artificial Sequence
  Synthetic 3′UTR (DNA), hOxa1 3′UTR
tgacttagttctgtgcgcattctggcaggaattctgtctcttcagagactcatcctcaaaacaagacttgacactgtgtccttgccccagtcctaggaactgtggcacacagagtagttcattttaaaaacggattactagaaacactcttgtactttagtttataagagagcactgggtagccaagtgatcttcccattcacagagttagtaaacctctgtactactagctg

SEQ ID NO: 72 - RNA, 233 nt, Artificial Sequence
  Synthetic 3′UTR (RNA), hOxa1 3′UTR
ugacuuaguucugugcgcauucuggcaggaauucugucucuucagagacucauccucaaaacaagacuugacacuguguccuugccccaguccuaggaacuguggcacacagaguaguucauuuuaaaaacggauuacuagaaacacucuuguacuuuaguuuauaagagagcacuggguagccaagugaucuucccauucacagaguuaguaaaccucuguacuacuagcug

SEQ ID NO: 73 - DNA, 138 bp, Artificial Sequence
  Synthetic 3′UTR (DNA), hOxa1_short 3′UTR
tgaactgtggcacacagagtagttcattttaaaaacggatttcgataaacactcttgtactttagtttataagagagcactgggtagccaagtgatcttcccattcacagagttagtaaacctctgtactactagctg

SEQ ID NO: 74 - RNA, 138 nt, Artificial Sequence
  Synthetic 3′UTR (RNA), hOxa1_short 3′UTR
ugaacuguggcacacagaguaguucauuuuaaaaacggauuucgauaaacacucuuguacuuuaguuuauaagagagcacuggguagccaagugaucuucccauucacagaguuaguaaaccucuguacuacuagcug

SEQ ID NO: 75 - DNA, 167 bp, Artificial Sequence
  Synthetic 3′UTR (DNA), mt-rpS12′1 3UTR
tgacagaagaagtgacggctgggggcacagtgggctgggcgcccctgcagaactagaaccttccgctcctggctgccacagggtcctccgtagctggcctttgcgcctgtagaggcagccactctaggattcaagtcctggctccgcctcttccatcaggaccacta

SEQ ID NO: 76 - RNA, 167 nt, Artificial Sequence
  Synthetic 3′UTR (RNA), mt-rpS12′1 3UTR
ugacagaagaagugacggcugggggcacagugggcugggcgccccugcagaacuagaaccuuccgcuccuggcugccacaggguccuccguagcuggccuuugcgccuguagaggcagccacucuaggauucaaguccuggcuccgccucuuccaucaggaccacua

SEQ ID NO: 77 - DNA, 166 bp, Artificial Sequence
  Synthetic 3′UTR (DNA), mSOD2 3UTR (DNA)
tgaacctcactcacggccacattgagtgccaggctccgggctggtttatagtagtgtagagcattgcagcacttagactggggtgctgtagtctttattgtagtctttccacatacctgataattcttagataatttcttattttaattaaatctattcttaggct

SEQ ID NO: 78 - RNA, 166 nt, Artificial Sequence
  Synthetic 3′UTR (RNA), mSOD2 3UTR (RNA)
ugaaccucacucacggccacauugagugccaggcuccgggcugguuuauaguaguguagagcauugcagcacuuagacuggggugcuguagucuuuauuguagucuuuccacauaccugauaauucuuagauaauuucuuauuuuaauuaaaucuauucuuaggcu

SEQ ID NO: 79 - RNA, 67 nt, Artificial Sequence
  Synthetic 5′UTR (RNA), CG2-5′UTR A-start
aggaaauaagagagaaaagaagaguaagaagaaauauaagacccgcccgccccgccccgccgccacc

SEQ ID NO: 80 - RNA, 61 nt, Artificial Sequence
  Synthetic 5′UTR (RNA), CG2-5′UTR Minus leader
uaagagagaaaagaagaguaagaagaaauauaagacccgcccgccccgccccgccgccacc

SEQ ID NO: 81 - RNA, 15 nt, Artificial Sequence
  Synthetic 5′UTR, KT1-UTR A-start
aggcccgccgccaac

SEQ ID NO: 82 - RNA, 15 nt, Artificial Sequence
  Synthetic 5′UTR, KT2-UTR A-start
aggcccgccgccacc

SEQ ID NO: 83 - RNA, 15 nt, Artificial Sequence
  Synthetic 5′UTR, KT3-UTR A-start
aggcccgccgccgac

SEQ ID NO: 84 - RNA, 15 nt, Artificial Sequence
  Synthetic 5′UTR, KT4-UTR A-start
aggcccgccgccgcc

SEQ ID NO: 85 - RNA, 21 nt, Artificial Sequence
  Synthetic 5′UTR, GCC3-ExtKozak A-start
aggaaagccgccgccgccacc

SEQ ID NO: 86 - RNA, 15 nt, Artificial Sequence
  Synthetic 5′UTR, GCC3-ExtKozak Minus leader
gccgccgccgccacc

SEQ ID NO: 87 - DNA, 176 bp, Artificial Sequence
  Synthetic 5′UTR (DNA), S065 core
cctcatatccaggctcaagaatagagctcagtgttttgttgtttaatcattccgacgtgttttgcgatattcgcgcaaagcagccagtcgcgcgcttgcttttaagtagagttgtttttccacccgtttgccaggcatctttaatttaacatatttttatttttcaggctaaccta

SEQ ID NO: 88 - DNA, 192 bp, Artificial Sequence
  Synthetic 5′UTR (DNA), S065
gggagacctcatatccaggctcaagaatagagctcagtgttttgttgtttaatcattccgacgtgttttgcgatattcgcgcaaagcagccagtcgcgcgcttgcttttaagtagagttgtttttccacccgtttgccaggcatctttaatttaacatatttttatttttcaggctaacctaaagcagagaa

SEQ ID NO: 89 - RNA, 192 nt, Artificial Sequence
  Synthetic 5′UTR (RNA), S065 A-start
aggagaccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuaaagcagagaa

SEQ ID NO: 90 - RNA, 186 nt, Artificial Sequence
  Synthetic 5′UTR (RNA), S065 core Minus leader
ccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuaaagcagagaa

SEQ ID NO: 91 - DNA, 192 bp, Artificial Sequence
  Synthetic 5′UTR (DNA), combo3_S065 (S065 core extended Kozak)
gggagacctcatatccaggctcaagaatagagctcagtgttttgttgtttaatcattccgacgtgttttgcgatattcgcgcaaagcagccagtcgcgcgcttgcttttaagtagagttgtttttccacccgtttgccaggcatctttaatttaacatatttttatttttcaggctaacctacgccgccacc

SEQ ID NO: 92 - RNA, 192 nt, Artificial Sequence
  Synthetic 5′UTR (RNA), combo3_S065 (S065 core extended Kozak) A-start
aggagaccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuacgccgccacc

SEQ ID NO: 93 - RNA, 186 nt, Artificial Sequence
  Synthetic 5′UTR (RNA), combo3_S065 (S065 core extended Kozak) Minus leader
ccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuacgccgccacc

SEQ ID NO: 94 - DNA, 205 bp, Artificial Sequence
  Synthetic 5′UTR (DNA), combo1_S065 (CR3)
gggaaaccccccacccccgcctcatatccaggctcaagaatagagctcagtgttttgttgtttaatcattccgacgtgttttgcgatattcgcgcaaagcagccagtcgcgcgcttgcttttaagtagagttgtttttccacccgtttgccaggcatctttaatttaacatatttttatttttcaggctaacctaaagcagagaa

SEQ ID NO: 95 - RNA, 205 nt, Artificial Sequence
  Synthetic 5′UTR (RNA), combo1_S065 (CR3) A-start
aggaaaccccccacccccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuaaagcagagaa

SEQ ID NO: 96 - RNA, 199 nt, Artificial Sequence
  Synthetic 5′UTR (RNA), combo1_S065 (CR3) Minus leader
ccccccacccccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuaaagcagagaa

SEQ ID NO: 97 - DNA, 204 bp, Artificial Sequence
  Synthetic 5′UTR (DNA), combo2_S065 (CR5)
gggaaatccccacaaccgcctcatatccaggctcaagaatagagctcagtgttttgttgtttaatcattccgacgtgttttgcgatattcgcgcaaagcagccagtcgcgcgcttgcttttaagtagagttgtttttccacccgtttgccaggcatctttaatttaacatatttttatttttcaggctaacctaaagcagagaa

SEQ ID NO: 98 - RNA, 204 nt, Artificial Sequence
  Synthetic 5′UTR (RNA), combo2_S065 (CR5) A-start
aggaaauccccacaaccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuaaagcagagaa

SEQ ID NO: 99 - RNA, 198 nt, Artificial Sequence
  Synthetic 5′UTR (RNA), combo2_S065 (CR5) Minus leader
uccccacaaccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuaaagcagagaa

SEQ ID NO: 100 - DNA, 205 bp, Artificial Sequence
  Synthetic 5′UTR (DNA), combo4_S065 (CR3)
gggaaaccccccacccccgcctcatatccaggctcaagaatagagctcagtgttttgttgtttaatcattccgacgtgttttgcgatattcgcgcaaagcagccagtcgcgcgcttgcttttaagtagagttgtttttccacccgtttgccaggcatctttaatttaacatatttttatttttcaggctaacctacgccgccacc

SEQ ID NO: 101 - RNA, 205 nt, Artificial Sequence
  Synthetic 5′UTR (RNA), combo4_S065 (CR3) A-start
aggaaaccccccacccccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuacgccgccacc

SEQ ID NO: 102 - RNA, 199 nt, Artificial Sequence
  Synthetic 5′UTR (RNA), combo4_S065 (CR3) Minus leader
ccccccacccccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuacgccgccacc

SEQ ID NO: 103 - DNA, 204 bp, Artificial Sequence
  Synthetic 5′UTR (DNA), F153, combo5_S065 (CR5)
gggaaatccccacaaccgcctcatatccaggctcaagaatagagctcagtgttttgttgtttaatcattccgacgtgttttgcgatattcgcgcaaagcagccagtcgcgcgcttgcttttaagtagagttgtttttccacccgtttgccaggcatctttaatttaacatatttttatttttcaggctaacctacgccgccacc

SEQ ID NO: 104 - RNA, 204 nt, Artificial Sequence
  Synthetic 5′UTR (RNA), F153, combo5_S065 (CR5) A-start
aggaaauccccacaaccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuacgccgccacc

SEQ ID NO: 105 - RNA, 198 nt, Artificial Sequence
  Synthetic 5′UTR (RNA), F153, combo5_S065 (CR5) Minus leader
uccccacaaccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuacgccgccacc

SEQ ID NO: 106 - DNA, 76 bp, Artificial Sequence
  Synthetic 5′UTR (DNA), combo1_V1.1 (CR3, V1)
gggaaaccccccacccccggggaaataagagagaaaagaagagtaagaagaaatataagaccccggcgccgccacc

SEQ ID NO: 107 - RNA, 76 nt, Artificial Sequence
  Synthetic 5′UTR (RNA), combo1_V1.1 (CR3, V1) A-start
aggaaaccccccacccccggggaaauaagagagaaaagaagaguaagaagaaauauaagaccccggcgccgccacc

SEQ ID NO: 108 - RNA, 70 nt, Artificial Sequence
  Synthetic 5′UTR (RNA), combo1_V1.1 (CR3, V1) Minus leader
ccccccacccccggggaaauaagagagaaaagaagaguaagaagaaauauaagaccccggcgccgccacc

SEQ ID NO: 109 - DNA, 75 bp, Artificial Sequence
  Synthetic 5′UTR (DNA), combo2_V1.1 (CR5, V1)
gggaaatccccacaaccggggaaataagagagaaaagaagagtaagaagaaatataagaccccggcgccgccacc

SEQ ID NO: 110 - RNA, 75 nt, Artificial Sequence
  Synthetic 5′UTR (RNA), combo2_V1.1 (CR5, V1) A-start
aggaaauccccacaaccggggaaauaagagagaaaagaagaguaagaagaaauauaagaccccggcgccgccacc

SEQ ID NO: 111 - RNA, 69 nt, Artificial Sequence
  Synthetic 5′UTR (RNA), combo2_V1.1 (CR5, V1) Minus leader
uccccacaaccggggaaauaagagagaaaagaagaguaagaagaaauauaagaccccggcgccgccacc

SEQ ID NO: 112 - DNA, 33 bp, Artificial Sequence
  Synthetic 5′UTR (DNA), CrichCR4 + GCC3-ExtKozak (CR4, GCC3)
gggaaacccccctaagccgccgccgccgccacc

SEQ ID NO: 113 - RNA, 33 nt, Artificial Sequence
  Synthetic 5′UTR (RNA), CrichCR4 + GCC3-ExtKozak (CR4, GCC3) A-start
aggaaaccccccuaagccgccgccgccgccacc

SEQ ID NO: 114 - RNA, 27 nt, Artificial Sequence
  Synthetic 5′UTR (RNA), CrichCR4 + GCC3-ExtKozak (CR4, GCC3) Minus leader
ccccccuaagccgccgccgccgccacc

SEQ ID NO: 115 - DNA, 77 bp, Artificial Sequence
  Synthetic 5′UTR (DNA), F856, RNAseP_p1 (V1, RNAseP)
gggaaataagagagaaaagaagagtaagaagaaatataagatctccctgagcttcagggagccccggcgccgccacc

SEQ ID NO: 116 - RNA, 77 nt, Artificial Sequence
  Synthetic 5′UTR (RNA), F856, RNAseP_p1 (V1, RNAseP)
gggaaauaagagagaaaagaagaguaagaagaaauauaagaucucccugagcuucagggagccccggcgccgccacc

SEQ ID NO: 117 - RNA, 77 nt, Artificial Sequence
  Synthetic 5′UTR (RNA), RNAseP_p1 (V1, RNAseP) A-start
aggaaauaagagagaaaagaagaguaagaagaaauauaagaucucccugagcuucagggagccccggcgccgccacc

SEQ ID NO: 118 - RNA, 71 nt, Artificial Sequence
  Synthetic 5′UTR (RNA), RNAseP_p1 (V1, RNAseP) Minus leader
uaagagagaaaagaagaguaagaagaaauauaagaucucccugagcuucagggagccccggcgccgccacc

SEQ ID NO: 119 - DNA, 77 bp, Artificial Sequence
  Synthetic 5′UTR (DNA), RNAse P-2 (V1, RNAseP)
gggaaatctccctgagcttcagggagtaagagagaaaagaagagtaagaagaaatataagaccccggcgccgccacc

SEQ ID NO: 120 - RNA, 77 nt, Artificial Sequence
  Synthetic 5′UTR (RNA), RNAse P-2 (V1, RNAseP)
gggaaaucucccugagcuucagggaguaagagagaaaagaagaguaagaagaaauauaagaccccggcgccgccacc

SEQ ID NO: 121 - RNA, 77 nt, Artificial Sequence
  Synthetic 5′UTR (RNA), RNAse P-2 (V1, RNAseP) A-start
aggaaaucucccugagcuucagggaguaagagagaaaagaagaguaagaagaaauauaagaccccggcgccgccacc

SEQ ID NO: 122 - RNA, 71 nt, Artificial Sequence
  Synthetic 5′UTR (RNA), RNAse P-2 (V1, RNAseP) Minus leader
ucucccugagcuucagggaguaagagagaaaagaagaguaagaagaaauauaagaccccggcgccgccacc

SEQ ID NO: 123 - DNA, 77 bp, Artificial Sequence
  Synthetic 5′UTR (DNA), RNAseP_p3 (V1, RNAseP)
gggaaataagagagaaaagaagatctccctgagcttcagggaggtaagaagaaatataagaccccggcgccgccacc

SEQ ID NO: 124 - RNA, 77 nt, Artificial Sequence
  Synthetic 5′UTR (RNA), RNAseP_p3 (V1, RNAseP)
gggaaauaagagagaaaagaagaucucccugagcuucagggagguaagaagaaauauaagaccccggcgccgccacc

SEQ ID NO: 125 - RNA, 77 nt, Artificial Sequence
  Synthetic 5′UTR (RNA), RNAseP_p3 (V1, RNAseP) A-start
aggaaauaagagagaaaagaagaucucccugagcuucagggagguaagaagaaauauaagaccccggcgccgccacc

SEQ ID NO: 126 - RNA, 71 nt, Artificial Sequence
  Synthetic 5′UTR (RNA), RNAseP_p3 (V1, RNAseP) Minus leader
uaagagagaaaagaagaucucccugagcuucagggagguaagaagaaauauaagaccccggcgccgccacc

SEQ ID NO: 127 - DNA, 90 bp, Artificial Sequence
  Synthetic 5′UTR (DNA), F593, combo1_P2_p1 (V1, CR3, RNAseP)
gggaaaccccccacccccgtaagagagaaaagaagagtaagaagaaatataagatctccctgagcttcagggagccccggcgccgccacc

SEQ ID NO: 128 - RNA, 90 nt, Artificial Sequence
  Synthetic 5′UTR (RNA), F593, combo1_P2_p1 (V1, CR3, RNAseP)
gggaaaccccccacccccguaagagagaaaagaagaguaagaagaaauauaagaucucccugagcuucagggagccccggcgccgccacc

SEQ ID NO: 129 - RNA, 90 nt, Artificial Sequence
  Synthetic 5′UTR (RNA), F593, combo1_P2_p1 (V1, CR3, RNAseP) A-start
aggaaaccccccacccccguaagagagaaaagaagaguaagaagaaauauaagaucucccugagcuucagggagccccggcgccgccacc

SEQ ID NO: 130 - RNA, 84 nt, Artificial Sequence
  Synthetic 5′UTR (RNA), F593, combo1_P2_p1 (V1, CR3, RNAseP) Minus leader
ccccccacccccguaagagagaaaagaagaguaagaagaaauauaagaucucccugagcuucagggagccccggcgccgccacc

SEQ ID NO: 131 - DNA, 90 bp, Artificial Sequence
  Synthetic 5′UTR (DNA), combo1_P2_p2  (V1, CR3, RNAseP)
gggaaaccccccacccccgtctccctgagcttcagggagtaagagagaaaagaagagtaagaagaaatataagaccccggcgccgccacc

SEQ ID NO: 132 - RNA, 90 nt, Artificial Sequence
  Synthetic 5′UTR (RNA), combo1_P2_p2 (V1, CR3, RNAseP)
gggaaaccccccacccccgucucccugagcuucagggaguaagagagaaaagaagaguaagaagaaauauaagaccccggcgccgccacc

SEQ ID NO: 133 - RNA, 90 nt, Artificial Sequence
  Synthetic 5′UTR (RNA), combo1_P2_p2 (V1, CR3, RNAseP) A-start
aggaaaccccccacccccgucucccugagcuucagggaguaagagagaaaagaagaguaagaagaaauauaagaccccggcgccgccacc

SEQ ID NO: 134 - RNA, 84 nt, Artificial Sequence
  Synthetic 5′UTR (RNA), combo1_P2_p2 (V1, CR3, RNAseP) Minus leader
ccccccacccccgucucccugagcuucagggaguaagagagaaaagaagaguaagaagaaauauaagaccccggcgccgccacc

SEQ ID NO: 135 - DNA, 90 bp, Artificial Sequence
  Synthetic 5′UTR (DNA), combo1_P2_p3 (V1, CR3, RNAseP)
gggaaaccccccacccccgtaagagagaaaagaagatctccctgagcttcagggaggtaagaagaaatataagaccccggcgccgccacc

SEQ ID NO: 136 - RNA, 90 nt, Artificial Sequence
  Synthetic 5′UTR (RNA), combo1_P2_p3 (V1, CR3, RNAseP)
gggaaaccccccacccccguaagagagaaaagaagaucucccugagcuucagggagguaagaagaaauauaagaccccggcgccgccacc

SEQ ID NO: 137 - RNA, 90 nt, Artificial Sequence
  Synthetic 5′UTR (RNA), combo1_P2_p3 (V1, CR3, RNAseP) A-start
aggaaaccccccacccccguaagagagaaaagaagaucucccugagcuucagggagguaagaagaaauauaagaccccggcgccgccacc

SEQ ID NO: 138 - RNA, 84 nt, Artificial Sequence
  Synthetic 5′UTR (RNA), combo1_P2_p3 (V1, CR3, RNAseP) Minus leader
ccccccacccccguaagagagaaaagaagaucucccugagcuucagggagguaagaagaaauauaagaccccggcgccgccacc

SEQ ID NO: 139 - DNA, 89 bp, Artificial Sequence
  Synthetic 5′UTR (DNA), combo2_P2_p2 (V1, CR5, RNAseP)
gggaaatccccacaaccgtctccctgagcttcagggagtaagagagaaaagaagagtaagaagaaatataagaccccggcgccgccacc

SEQ ID NO: 140 - RNA, 89 nt, Artificial Sequence
  Synthetic 5′UTR (RNA), combo2_P2_p2 (V1, CR5, RNAseP)
gggaaauccccacaaccgucucccugagcuucagggaguaagagagaaaagaagaguaagaagaaauauaagaccccggcgccgccacc

SEQ ID NO: 141 - RNA, 89 nt, Artificial Sequence
  Synthetic 5′UTR (RNA), combo2_P2_p2 (V1, CR5, RNAseP) A-start
aggaaauccccacaaccgucucccugagcuucagggaguaagagagaaaagaagaguaagaagaaauauaagaccccggcgccgccacc

SEQ ID NO: 142 - RNA, 83 nt, Artificial Sequence
  Synthetic 5′UTR (RNA), combo2_P2_p2 (V1, CR5, RNAseP) Minus leader
uccccacaaccgucucccugagcuucagggaguaagagagaaaagaagaguaagaagaaauauaagaccccggcgccgccacc

SEQ ID NO: 143 - DNA, 89 bp, Artificial Sequence
  Synthetic 5′UTR (DNA), combo2_P2_p3 (V1, CR5, RNAseP)
gggaaatccccacaaccgtaagagagaaaagaagatctccctgagcttcagggaggtaagaagaaatataagaccccggcgccgccacc

SEQ ID NO: 144 - RNA, 89 nt, Artificial Sequence
  Synthetic 5′UTR (RNA), combo2_P2_p3 (V1, CR5, RNAseP)
gggaaauccccacaaccguaagagagaaaagaagaucucccugagcuucagggagguaagaagaaauauaagaccccggcgccgccacc

SEQ ID NO: 145 - RNA, 89 nt, Artificial Sequence
  Synthetic 5′UTR (RNA), combo2_P2_p3 (V1, CR5, RNAseP) A-start
aggaaauccccacaaccguaagagagaaaagaagaucucccugagcuucagggagguaagaagaaauauaagaccccggcgccgccacc

SEQ ID NO: 146 - RNA, 83 nt, Artificial Sequence
  Synthetic 5′UTR (RNA), combo2_P2_p3 (V1, CR5, RNAseP) Minus leader
uccccacaaccguaagagagaaaagaagaucucccugagcuucagggagguaagaagaaauauaagaccccggcgccgccacc

SEQ ID NO: 147 - DNA, 240 bp, Artificial Sequence
  Synthetic 5′UTR (DNA), mt-rpS12 5UTR
gggagctggattcagcgtgtccgcgacctcacctttaggtcctgtgaggtcggtggaatcctggggtcctccaaatctaccaggccatctccccagtttcccagttcttcctgcgtgcgggcgagagtggttgggccctcgggaacccactcagagcgaggctaaatttacggagggactttctgttagcagcatgagggcctgtggttagacctatagagggacggcccaggtggcagg

SEQ ID NO: 148 - RNA, 240 nt, Artificial Sequence
  Synthetic 5′UTR (RNA), mt-rpS12 5UTR
gggagcuggauucagcguguccgcgaccucaccuuuagguccugugaggucgguggaauccugggguccuccaaaucuaccaggccaucuccccaguuucccaguucuuccugcgugcgggcgagagugguugggcccucgggaacccacucagagcgaggcuaaauuuacggagggacuuucuguuagcagcaugagggccugugguuagaccuauagagggacggcccagguggcagg

SEQ ID NO: 149 - DNA, 119 bp, Artificial Sequence
  Synthetic 3′UTR (DNA), 3′v1.0
tgataataggctggagcctcggtggccatgcttcttgccccttgggcctccccccagcccctcctccccttcctgcacccgtacccccgtggtctttgaataaagtctgagtgggcggc

SEQ ID NO: 150 - RNA, 119 nt, Artificial Sequence
  Synthetic 3′UTR (RNA), 3′v1.0
ugauaauaggcuggagccucgguggccaugcuucuugccccuugggccuccccccagccccuccuccccuuccugcacccguacccccguggucuuugaauaaagucugagugggcggc

SEQ ID NO: 151 - RNA, 110 nt, Artificial Sequence
  Synthetic 3′UTR (RNA), 3′v1.0 Minus stop
gcuggagccucgguggccaugcuucuugccccuugggccuccccccagccccuccuccccuuccugcacccguacccccguggucuuugaauaaagucugagugggcggc

SEQ ID NO: 152 - RNA, 119 nt, Artificial Sequence
  Synthetic 3′UTR (RNA), 3′v1.0 altered stop
uaauaguaagcuggagccucgguggccaugcuucuugccccuugggccuccccccagccccuccuccccuuccugcacccguacccccguggucuuugaauaaagucugagugggcggc

SEQ ID NO: 153 - RNA, 110 nt, Artificial Sequence
  Synthetic 3′UTR (RNA), 3′v1.1 Minus stop
gcuggagccucgguggccuagcuucuugccccuugggccuccccccagccccuccuccccuuccugcacccguacccccguggucuuugaauaaagucugagugggcggc

SEQ ID NO: 154 - RNA, 119 nt, Artificial Sequence
  Synthetic 3′UTR (RNA), 3′v1.1 altered stop
uaauaguaagcuggagccucgguggccuagcuucuugccccuugggccuccccccagccccuccuccccuuccugcacccguacccccguggucuuugaauaaagucugagugggcggc

SEQ ID NO: 155 - protein, 8 aa, Artificial Sequence
  Synthetic Epitope Tag, FLAG
DYKDDDDK

SEQ ID NO: 156 - protein, 21 aa, Artificial Sequence
  Synthetic Epitope Tag, 3XFLAG
DYKDHDGDYKDHDIDYKDDDK

SEQ ID NO: 157 - RNA, 85 nt, Artificial Sequence
  Synthetic miR, miR-122
ccuuagcagagcuguggagugugacaaugguguuugugucuaaacuaucaaacgccauuaucacacuaaauagcuacugcuaggc

SEQ ID NO: 158 - RNA, 22 nt, Artificial Sequence
  Synthetic miR, miR-122-3p
aacgccauuaucacacuaaaua

SEQ ID NO: 159 - RNA, 22 nt, Artificial Sequence
  Synthetic miR binding site, miR-122-3p binding site
uauuuagugugauaauggcguu

SEQ ID NO: 160 - RNA, 22 nt, Artificial Sequence
  Synthetic miR, miR-122-5p
uggagugugacaaugguguuug

SEQ ID NO: 161 - RNA, 22 nt, Artificial Sequence
  Synthetic miR binding site, miR-122-5p binding site
caaacaccauugucacacucca

SEQ ID NO: 162 - DNA, 142 bp, Artificial Sequence
  Synthetic 3′UTR (DNA), 3′v1.1m142  3′v1.1 + miR-142-3p binding site
tgataataggctggagcctcggtggcctagcttcttgccccttgggcctccccccagcccctcctccccttcctgcacccgtaccccctccataaagtaggaaacactacagtggtctttgaataaagtctgagtgggcggc

SEQ ID NO: 163 - RNA, 142 nt, Artificial Sequence
  Synthetic 3′UTR (RNA), 3′v1.1m142  3′v1.1 + miR-142-3p binding site
ugauaauaggcuggagccucgguggccuagcuucuugccccuugggccuccccccagccccuccuccccuuccugcacccguacccccuccauaaaguaggaaacacuacaguggucuuugaauaaagucugagugggcggc

SEQ ID NO: 164 - DNA, 206 bp, Artificial Sequence
  Synthetic 5′UTR (DNA), mSOD2 5UTR
ggggagacgttgccttcccaggatgccgctccgttatgcgccgggccgtccgtgtcgccgtcctcccctccgctgatgggcgctgcgggcagggtcaccgcttcgctgtgtccttgcggacgccgggcggacgctgcctagcagacgcgcgcctgcgagcgaacggccgtgttctgaggagagcagcggtcgtgtaaacctcaata

SEQ ID NO: 165 - RNA, 206 nt, Artificial Sequence
  Synthetic 5′UTR (RNA), mSOD2 5UTR
ggggagacguugccuucccaggaugccgcuccguuaugcgccgggccguccgugucgccguccuccccuccgcugaugggcgcugcgggcagggucaccgcuucgcuguguccuugcggacgccgggcggacgcugccuagcagacgcgcgccugcgagcgaacggccguguucugaggagagcagcggucguguaaaccucaaua

SEQ ID NO: 166 - RNA, 173 nt, Artificial Sequence
  Synthetic 3′UTR (RNA), mt-rpS12′1 3UTR
uaauaguaacagaagaagugacggcugggggcacagugggcugggcgccccugcagaacuagaaccuuccgcuccuggcugccacaggguccuccguagcuggccuuugcgccuguagaggcagccacucuaggauucaaguccuggcuccgccucuuccaucaggaccacua

SEQ ID NO: 167 - RNA, 172 nt, Artificial Sequence
  Synthetic 3′UTR (RNA), mSOD2 3UTR altered stop
uaauaguaaaccucacucacggccacauugagugccaggcuccgggcugguuuauaguaguguagagcauugcagcacuuagacuggggugcuguagucuuuauuguagucuuuccacauaccugauaauucuuagauaauuucuuauuuuaauuaaaucuauucuuaggcu

SEQ ID NO: 168 - RNA, 142 nt, Artificial Sequence
  Synthetic 3′UTR (RNA), 3′v1.1m142  3′v1.1 + miR-142-3p binding site  Altered stop
uaauaguaagcuggagccucgguggccuagcuucuugccccuugggccuccccccagccccuccuccccuuccugcacccguacccccuccauaaaguaggaaacacuacaguggucuuugaauaaagucugagugggcggc

SEQ ID NO: 169 - DNA, 241 bp, Artificial Sequence
  Synthetic 3′UTR (DNA), Sod2_3 (N3xstop_mSod2_3x-142-3p_3)
taatagtaaacctcactcacggccacattgagtgccaggctccgggctggtttatagtagtgtagagcattgcagcacttagactggggtgctgtagtctttattgtagtctttccacatacctgataattcttagataatttcttattttaattccataaagtaggaaacactacataaatctccataaagtaggaaacactacatattcttccataaagtaggaaacactacataggct

SEQ ID NO: 170 - RNA, 241 nt, Artificial Sequence
  Synthetic 3′UTR (RNA), Sod2_3 (N3xstop_mSod2_3x-142-3p_3)
uaauaguaaaccucacucacggccacauugagugccaggcuccgggcugguuuauaguaguguagagcauugcagcacuuagacuggggugcuguagucuuuauuguagucuuuccacauaccugauaauucuuagauaauuucuuauuuuaauuccauaaaguaggaaacacuacauaaaucuccauaaaguaggaaacacuacauauucuuccauaaaguaggaaacacuacauaggcu

SEQ ID NO: 171 - DNA, 241 bp, Artificial Sequence
  Synthetic 3′UTR (DNA), Sod2_5 (N3xstop_mSod2_3x-142-3p_5)
taatagtaaacctcatccataaagtaggaaacactacactcacgtccataaagtaggaaacactacagccacatccataaagtaggaaacactacattgagtgccaggctccgggctggtttatagtagtgtagagcattgcagcacttagactggggtgctgtagtctttattgtagtctttccacatacctgataattcttagataatttcttattttaattaaatctattcttaggct

SEQ ID NO: 172 - RNA, 241 nt, Artificial Sequence
  Synthetic 3′UTR (RNA), Sod2_5 (N3xstop_mSod2_3x-142-3p_5)
uaauaguaaaccucauccauaaaguaggaaacacuacacucacguccauaaaguaggaaacacuacagccacauccauaaaguaggaaacacuacauugagugccaggcuccgggcugguuuauaguaguguagagcauugcagcacuuagacuggggugcuguagucuuuauuguagucuuuccacauaccugauaauucuuagauaauuucuuauuuuaauuaaaucuauucuuaggcu

SEQ ID NO: 173 - DNA, 242 bp, Artificial Sequence
  Synthetic 3′UTR (DNA), rpS12_3 (N3xstop_mt-rpS12_3x-142-3p_3)
taatagtaacagaagaagtgacggctgggggcacagtgggctgggcgcccctgcagaactagaaccttccgctcctggctgccacagggtcctccgtagctggcctttgcgcctgtagaggcagccactctaggattcaagtcctggctccgccttccataaagtaggaaacactacacttccatccataaagtaggaaacactacatcaggatccataaagtaggaaacactacaccacta

SEQ ID NO: 174 - RNA, 242 nt, Artificial Sequence
  Synthetic 3′UTR (RNA), rpS12_3 (N3xstop_mt-rpS12_3x-142-3p_3)
uaauaguaacagaagaagugacggcugggggcacagugggcugggcgccccugcagaacuagaaccuuccgcuccuggcugccacaggguccuccguagcuggccuuugcgccuguagaggcagccacucuaggauucaaguccuggcuccgccuuccauaaaguaggaaacacuacacuuccauccauaaaguaggaaacacuacaucaggauccauaaaguaggaaacacuacaccacua

SEQ ID NO: 175 - DNA, 242 bp, Artificial Sequence
  Synthetic 3′UTR (DNA), rpS12_5 (N3xstop_mt-rpS12_3x-142-3p_5)
taatagtaacagaagtccataaagtaggaaacactacaaagtgatccataaagtaggaaacactacacggctgtccataaagtaggaaacactacaggggcacagtgggctgggcgcccctgcagaactagaaccttccgctcctggctgccacagggtcctccgtagctggcctttgcgcctgtagaggcagccactctaggattcaagtcctggctccgcctcttccatcaggaccacta

SEQ ID NO: 176 - RNA, 242 nt, Artificial Sequence
  Synthetic 3′UTR (RNA), rpS12_5 (N3xstop_mt-rpS12_3x-142-3p_5)
uaauaguaacagaaguccauaaaguaggaaacacuacaaagugauccauaaaguaggaaacacuacacggcuguccauaaaguaggaaacacuacaggggcacagugggcugggcgccccugcagaacuagaaccuuccgcuccuggcugccacaggguccuccguagcuggccuuugcgccuguagaggcagccacucuaggauucaaguccuggcuccgccucuuccaucaggaccacua

SEQ ID NO: 177 - RNA, 87 nt, Artificial Sequence
  Synthetic miR, mmiR-142
gacagugcagucacccauaaaguagaaagcacuacuaacagcacuggaggguguaguguuuccuacuuuauggaugaguguacugug

SEQ ID NO: 178 - RNA, 23 nt, Artificial Sequence
  Synthetic miR, mmiR-142-3p
uguaguguuuccuacuuuaugga

SEQ ID NO: 179 - RNA, 23 nt, Artificial Sequence
  Synthetic miR binding site, mmiR-142-3p binding site
uccauaaaguaggaaacacuaca

SEQ ID NO: 180 - RNA, 21 nt, Artificial Sequence
  Synthetic miR, mmiR-142-5p
cauaaaguagaaagcacuacu

SEQ ID NO: 181 - RNA, 21 nt, Artificial Sequence
  Synthetic miR binding site, mmiR-142-5p binding site
aguagugcuuucuacuuuaug

SEQ ID NO: 182 - RNA, 9 nt, Artificial Sequence
  Synthetic Stop codon, Triple stop 1
ugauaauag

SEQ ID NO: 183 - RNA, 9 nt, Artificial Sequence
  Synthetic Stop codon, Triple stop 2
uaauaguaa

SEQ ID NO: 184 - protein, 10 aa, Artificial Sequence
  Synthetic Epitope tag, Myc
EQKLISEEDL

SEQ ID NO: 185 - protein, 14 aa, Artificial Sequence
  Synthetic Epitope tag, V5
GKPIPNPLLGLDST

SEQ ID NO: 186 - protein, 9 aa, Artificial Sequence
  Synthetic Epitope tag, Hemagglutinin A (HA)
YPYDVPDYA

SEQ ID NO: 187 - protein, 6 aa, Artificial Sequence
  Synthetic Epitope tag, 6xHis tag
HHHHHH

SEQ ID NO: 188 - protein, 11 aa, Artificial Sequence
  Synthetic Epitope tag, HSV
QPELAPEDPED

SEQ ID NO: 189 - protein, 11 aa, Artificial Sequence
  Synthetic Epitope tag, VSV-G
YTDIEMNRLGK

SEQ ID NO: 190 - protein, 18 aa, Artificial Sequence
  Synthetic Epitope tag, NE
TKENPRSNQEESYDDNES

SEQ ID NO: 191 - protein, 15 aa, Artificial Sequence
  Synthetic Epitope tag, AViTag
GLNDIFEAQKIEWHE

SEQ ID NO: 192 - protein, 26 aa, Artificial Sequence
  Synthetic Epitope tag, Calmodulin
KRRWKKNFIAVSAANRFKKISSSGAL

SEQ ID NO: 193 - protein, 13 aa, Artificial Sequence
  Synthetic Epitope tag, E tag
GAPVPYPDPLEPR

SEQ ID NO: 194 - protein, 15 aa, Artificial Sequence
  Synthetic Epitope tag, S tag
KETAAAKFERQHMDS

SEQ ID NO: 195 - protein, 38 aa, Artificial Sequence
  Synthetic Epitope tag, SBP tag
MDEKTTGWRGGHVVEGLAGELEQLRARLEHHPQGQREP

SEQ ID NO: 196 - protein, 13 aa, Artificial Sequence
  Synthetic Epitope tag, Softag 1
SLAELLNAGLGGS

SEQ ID NO: 197 - protein, 8 aa, Artificial Sequence
  Synthetic Epitope tag, Softag 3
TQDPSRVG

SEQ ID NO: 198 - protein, 8 aa, Artificial Sequence
  Synthetic Epitope tag, Strep tag
WSHPQFEK

SEQ ID NO: 199 - protein, 10 aa, Artificial Sequence
  Synthetic Epitope tag, Ty tag
EVHTNQDPLD

SEQ ID NO: 200 - protein, 8 aa, Artificial Sequence
  Synthetic Epitope tag, Xpress tag
DLYDDDDK

SEQ ID NO: 201 - RNA, 47 nt, Artificial Sequence
  Synthetic 5UTR-001
gggagaucagagagaaaagaagaguaagaagaaauauaagagccacc

SEQ ID NO: 202 - RNA, 145 nt, Artificial Sequence
  Synthetic 5′UTR-002
ggaauaaaagucucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagcaac

SEQ ID NO: 203 - RNA, 42 nt, Artificial Sequence
  Synthetic 5′UTR-003
gggagacaagcuuggcauuccgguacuguugguaaagccacc

SEQ ID NO: 204 - RNA, 47 nt, Artificial Sequence
  Synthetic 5′UTR-004
gggaauuaacagagaaaagaagaguaagaagaaauauaagagccacc

SEQ ID NO: 205 - RNA, 47 nt, Artificial Sequence
  Synthetic 5′UTR-005
gggaaauuagacagaaaagaagaguaagaagaaauauaagagccacc

SEQ ID NO: 206 - RNA, 47 nt, Artificial Sequence
  Synthetic 5′UTR-006
gggaaauaagagaguaaagaacaguaagaagaaauauaagagccacc

SEQ ID NO: 207 - RNA, 47 nt, Artificial Sequence
  Synthetic 5′UTR-007
gggaaaaaagagagaaaagaagacuaagaagaaauauaagagccacc

SEQ ID NO: 208 - RNA, 47 nt, Artificial Sequence
  Synthetic 5′UTR-008
gggaaauaagagagaaaagaagaguaagaagauauauaagagccacc

SEQ ID NO: 209 - RNA, 47 nt, Artificial Sequence
  Synthetic 5′UTR-009
gggaaauaagagacaaaacaagaguaagaagaaauauaagagccacc

SEQ ID NO: 210 - RNA, 47 nt, Artificial Sequence
  Synthetic 5′UTR-010
gggaaauuagagaguaaagaacaguaaguagaauuaaaagagccacc

SEQ ID NO: 211 - RNA, 47 nt, Artificial Sequence
  Synthetic 5′UTR-011
gggaaauaagagagaauagaagaguaagaagaaauauaagagccacc

SEQ ID NO: 212 - RNA, 47 nt, Artificial Sequence
  Synthetic 5′UTR-012
gggaaauaagagagaaaagaagaguaagaagaaaauuaagagccacc

SEQ ID NO: 213 - RNA, 47 nt, Artificial Sequence
  Synthetic 5′UTR-013
gggaaauaagagagaaaagaagaguaagaagaaauuuaagagccacc

SEQ ID NO: 214 - RNA, 92 nt, Artificial Sequence
  Synthetic 5′UTR-014
ucaagcuuuuggacccucguacagaagcuaauacgacucacuauagggaaauaagagagaaaagaagaguaagaagaaauauaagagccacc

SEQ ID NO: 215 - RNA, 140 nt, Artificial Sequence
  Synthetic 5′UTR-015
ggacagaucgccuggagacgccauccacgcuguuuugaccuccauagaagacaccgggaccgauccagccuccgcggccgggaacggugcauuggaacgcggauuccccgugccaagagugacucaccguccuugacacg

SEQ ID NO: 216 - RNA, 42 nt, Artificial Sequence
  Synthetic 5′UTR-016
ggcgcugccuacggagguggcagccaucuccuucucggcauc

SEQ ID NO: 217 - RNA, 371 nt, Artificial Sequence
  Synthetic 3′ UTR-001 - Creatine Kinase
gcgccugcccaccugccaccgacugcuggaacccagccagugggagggccuggcccaccagaguccugcucccucacuccucgccccgcccccugucccagagucccaccugggggcucucuccacccuucucagaguuccaguuucaaccagaguuccaaccaaugggcuccauccucuggauucuggccaaugaaauaucucccuggcaggguccucuucuuuucccagagcuccaccccaaccaggagcucuaguuaauggagagcucccagcacacucggagcuugugcuuugucuccacgcaaagcgauaaauaaaagcauugguggccuuuggucuuugaauaaagccugaguaggaagucuaga

SEQ ID NO: 218 - RNA, 568 nt, Artificial Sequence
  Synthetic 3′ UTR-002 - Myoglobin
gccccugccgcucccacccccacccaucugggccccggguucaagagagagcggggucugaucucguguagccauauagaguuugcuucugagugucugcuuuguuuaguagaggugggcaggaggagcugaggggcuggggcugggguguugaaguuggcuuugcaugcccagcgaugcgccucccugugggaugucaucacccugggaaccgggaguggcccuuggcucacuguguucugcaugguuuggaucugaauuaauuguccuuucuucuaaaucccaaccgaacuucuuccaaccuccaaacuggcuguaaccccaaauccaagccauuaacuacaccugacaguagcaauugucugauuaaucacuggccccuugaagacagcagaaugucccuuugcaaugaggaggagaucugggcugggcgggccagcuggggaagcauuugacuaucuggaacuugugugugccuccucagguauggcagugacucaccugguuuuaauaaaacaaccugcaacaucucauggucuuugaauaaagccugaguaggaagucuaga

SEQ ID NO: 219 - RNA, 289 nt, Artificial Sequence
  Synthetic 3′ UTR-003 - alpha-actin
acacacuccaccuccagcacgcgacuucucaggacgacgaaucuucucaaugggggggcggcugagcuccagccaccccgcagucacuuucuuuguaacaacuuccguugcugccaucguaaacugacacaguguuuauaacguguacauacauuaacuuauuaccucauuuuguuauuuuucgaaacaaagcccuguggaagaaaauggaaaacuugaagaagcauuaaagucauucuguuaagcugcguaaauggucuuugaauaaagccugaguaggaagucuaga

SEQ ID NO: 220 - RNA, 379 nt, Artificial Sequence
  Synthetic 3′ UTR-004 - Albumin
caucacauuuaaaagcaucucagccuaccaugagaauaagagaaagaaaaugaagaucaaaagcuuauucaucuguuuuucuuuuucguugguguaaagccaacacccugucuaaaaaacauaaauuucuuuaaucauuuugccucuuuucucugugcuucaauuaauaaaaaauggaaagaaucuaauagagugguacagcacuguuauuuuucaaagauguguugcuauccugaaaauucuguagguucuguggaaguuccaguguucucucuuauuccacuucgguagaggauuucuaguuucuugugggcuaauuaaauaaaucauuaauacucuucuaauggucuuugaauaaagccugaguaggaagucuaga

SEQ ID NO: 221 - RNA, 118 nt, Artificial Sequence
  Synthetic 3′ UTR-005 - alpha-globin
gcugccuucugcggggcuugccuucuggccaugcccuucuucucucccuugcaccuguaccucuuggucuuugaauaaagccugaguaggaaggcggccgcucgagcaugcaucuaga

SEQ ID NO: 222 - RNA, 908 nt, Artificial Sequence
  Synthetic 3′ UTR-006 - G-CSF
gccaagcccuccccaucccauguauuuaucucuauuuaauauuuaugucuauuuaagccucauauuuaaagacagggaagagcagaacggagccccaggccucuguguccuucccugcauuucugaguuucauucuccugccuguagcagugagaaaaagcuccuguccucccauccccuggacugggagguagauagguaaauaccaaguauuuauuacuaugacugcuccccagcccuggcucugcaaugggcacugggaugagccgcugugagccccugguccugaggguccccaccugggacccuugagaguaucaggucucccacgugggagacaagaaaucccuguuuaauauuuaaacagcaguguuccccaucuggguccuugcaccccucacucuggccucagccgacugcacagcggccccugcauccccuuggcugugaggccccuggacaagcagagguggccagagcugggaggcauggcccuggggucccacgaauuugcuggggaaucucguuuuucuucuuaagacuuuugggacaugguuugacucccgaacaucaccgacgcgucuccuguuuuucuggguggccucgggacaccugcccugcccccacgagggucaggacugugacucuuuuuagggccaggcaggugccuggacauuugccuugcuggacggggacuggggaugugggagggagcagacaggaggaaucaugucaggccugugugugaaaggaagcuccacugucacccuccaccucuucaccccccacucaccaguguccccuccacugucacauuguaacugaacuucaggauaauaaaguguuugccuccauggucuuugaauaaagccugaguaggaaggcggccgcucgagcaugcaucuaga

SEQ ID NO: 223 - RNA, 835 nt, Artificial Sequence
  Synthetic 3′ UTR-007 - Col1a2; collagen, type I, alpha 2
acucaaucuaaauuaaaaaagaaagaaauuugaaaaaacuuucucuuugccauuucuucuucuucuuuuuuaacugaaagcugaauccuuccauuucuucugcacaucuacuugcuuaaauugugggcaaaagagaaaaagaaggauugaucagagcauugugcaauacaguuucauuaacuccuucccccgcucccccaaaaauuugaauuuuuuuuucaacacucuuacaccuguuauggaaaaugucaaccuuuguaagaaaaccaaaauaaaaauugaaaaauaaaaaccauaaacauuugcaccacuuguggcuuuugaauaucuuccacagagggaaguuuaaaacccaaacuuccaaagguuuaaacuaccucaaaacacuuucccaugagugugauccacauuguuaggugcugaccuagacagagaugaacugagguccuuguuuuguuuuguucauaauacaaaggugcuaauuaauaguauuucagauacuugaagaauguugauggugcuagaagaauuugagaagaaauacuccuguauugaguuguaucgugugguguauuuuuuaaaaaauuugauuuagcauucauauuuuccaucuuauucccaauuaaaaguaugcagauuauuugcccaaaucuucuucagauucagcauuuguucuuugccagucucauuuucaucuucuuccaugguuccacagaagcuuuguuucuugggcaagcagaaaaauuaaauuguaccuauuuuguauaugugagauguuuaaauaaauugugaaaaaaaugaaauaaagcauguuugguuuuccaaaagaacauau

SEQ ID NO: 224 - RNA, 297 nt, Artificial Sequence
  Synthetic 3′ UTR-008 - Col6a2; collagen, type VI, alpha 2
cgccgccgcccgggccccgcagucgagggucgugagcccaccccguccauggugcuaagcgggcccgggucccacacggccagcaccgcugcucacucggacgacgcccugggccugcaccucuccagcuccucccacgggguccccguagccccggcccccgcccagccccaggucuccccaggcccuccgcaggcugcccggccucccucccccugcagccaucccaaggcuccugaccuaccuggccccugagcucuggagcaagcccugacccaauaaaggcuuugaacccau

SEQ ID NO: 225 - RNA, 602 nt, Artificial Sequence
  Synthetic 3′ UTR-009 - RPN1; ribophorin I
ggggcuagagcccucuccgcacagcguggagacggggcaaggagggggguuauuaggauuggugguuuuguuuugcuuuguuuaaagccgugggaaaauggcacaacuuuaccucugugggagaugcaacacugagagccaaggggugggaguugggauaauuuuuauauaaaagaaguuuuuccacuuugaauugcuaaaaguggcauuuuuccuaugugcagucacuccucucauuucuaaaauagggacguggccaggcacgguggcucaugccuguaaucccagcacuuugggaggccgaggcaggcggcucacgaggucaggagaucgagacuauccuggcuaacacgguaaaacccugucucuacuaaaaguacaaaaaauuagcugggcgugguggugggcaccuguagucccagcuacucgggaggcugaggcaggagaaaggcaugaauccaagaggcagagcuugcagugagcugagaucacgccauugcacuccagccugggcaacaguguuaagacucugucucaaauauaaauaaauaaauaaauaaauaaauaaauaaauaaaaauaaagcgagauguugcccucaaa

SEQ ID NO: 226 - RNA, 785 nt, Artificial Sequence
  Synthetic 3′ UTR-010 - LRP1; low density lipoprotein receptor-related protein 1
ggcccugccccgucggacugcccccagaaagccuccugcccccugccagugaaguccuucagugagccccuccccagccagcccuucccuggccccgccggauguauaaauguaaaaaugaaggaauuacauuuuauaugugagcgagcaagccggcaagcgagcacaguauuauuucuccauccccucccugccugcuccuuggcacccccaugcugccuucagggagacaggcagggagggcuuggggcugcaccuccuacccucccaccagaacgcaccccacugggagagcugguggugcagccuuccccucccuguauaagacacuuugccaaggcucuccccucucgccccaucccugcuugcccgcucccacagcuuccugagggcuaauucugggaagggagaguucuuugcugccccugucuggaagacguggcucugggugagguaggcgggaaaggauggaguguuuuaguucuugggggaggccaccccaaaccccagccccaacuccaggggcaccuaugagauggccaugcucaaccccccucccagacaggcccucccugucuccagggcccccaccgagguucccagggcuggagacuuccucugguaaacauuccuccagccuccccuccccuggggacgccaaggaggugggccacacccaggaagggaaagcgggcagccccguuuuggggacgugaacguuuuaauaauuuuugcugaauuccuuuacaacuaaauaacacagauauuguuauaaauaaaauugu

SEQ ID NO: 227 - RNA, 3001 nt, Artificial Sequence
  Synthetic 3′ UTR-011 - Nnt1; cardiotrophin-like cytokine factor 1
auauuaaggaucaagcuguuagcuaauaaugccaccucugcaguuuugggaacaggcaaauaaaguaucaguauacauggugauguacaucuguagcaaagcucuuggagaaaaugaagacugaagaaagcaaagcaaaaacuguauagagagauuuuucaaaagcaguaaucccucaauuuuaaaaaaggauugaaaauucuaaaugucuuucugugcauauuuuuuguguuaggaaucaaaaguauuuuauaaaaggagaaagaacagccucauuuuagauguaguccuguuggauuuuuuaugccuccucaguaaccagaaauguuuuaaaaaacuaaguguuuaggauuucaagacaacauuauacauggcucugaaauaucugacacaauguaaacauugcaggcaccugcauuuuauguuuuuuuuuucaacaaaugugacuaauuugaaacuuuuaugaacuucugagcuguccccuugcaauucaaccgcaguuugaauuaaucauaucaaaucaguuuuaauuuuuuaaauuguacuucagagucuauauuucaagggcacauuuucucacuacuauuuuaauacauuaaaggacuaaauaaucuuucagagaugcuggaaacaaaucauuugcuuuauauguuucauuagaauaccaaugaaacauacaacuugaaaauuaguaauaguauuuuugaagaucccauuucuaauuggagaucucuuuaauuucgaucaacuuauaauguguaguacuauauuaagugcacuugaguggaauucaacauuugacuaauaaaaugaguucaucauguuggcaagugauguggcaauuaucucuggugacaaaagaguaaaaucaaauauuucugccuguuacaaauaucaaggaagaccugcuacuaugaaauagaugacauuaaucugucuucacuguuuauaauacggauggauuuuuuuucaaaucaguguguguuuugaggucuuauguaauugaugacauuugagagaaaugguggcuuuuuuuagcuaccucuuuguucauuuaagcaccaguaaagaucaugucuuuuuauagaaguguagauuuucuuugugacuuugcuaucgugccuaaagcucuaaauauaggugaaugugugaugaauacucagauuauuugucucucuauauaauuaguuugguacuaaguuucucaaaaaauuauuaacacaugaaagacaaucucuaaaccagaaaaagaaguaguacaaauuuuguuacuguaaugcucgcguuuagugaguuuaaaacacacaguaucuuuugguuuuauaaucaguuucuauuuugcugugccugagauuaagaucuguguaugugugugugugugugugugcguuuguguguuaaagcagaaaagacuuuuuuaaaaguuuuaagugauaaaugcaauuuguuaauugaucuuagaucacuaguaaacucagggcugaauuauaccauguauauucuauuagaagaaaguaaacaccaucuuuauuccugcccuuuuucuucucucaaaguaguuguaguuauaucuagaaagaagcaauuuugauuucuugaaaagguaguuccugcacucaguuuaaacuaaaaauaaucauacuuggauuuuauuuauuuuugucauaguaaaaauuuuaauuuauauauauuuuuauuuaguauuaucuuauucuuugcuauuugccaauccuuugucaucaauuguguuaaaugaauugaaaauucaugcccuguucauuuuauuuuacuuuauugguuaggauauuuaaaggauuuuuguauauauaauuucuuaaauuaauauuccaaaagguuaguggacuuagauuauaaauuauggcaaaaaucuaaaaacaacaaaaaugauuuuuauacauucuauuucauuauuccucuuuuuccaauaagucauacaauugguagauaugacuuauuuuauuuuuguauuauucacuauaucuuuaugauauuuaaguauaaauaauuaaaaaaauuuauuguaccuuauagucugucaccaaaaaaaaaaaauuaucuguagguagugaaaugcuaauguugauuugucuuuaagggcuuguuaacuauccuuuauuuucucauuugucuuaaauuaggaguuuguguuuaaauuacucaucuaagcaaaaaauguauauaaaucccauuacuggguauauacccaaaggauuauaaaucaugcugcuauaaagacacaugcacacguauguuuauugcagcacuauucacaauagcaaagacuuggaaccaacccaaauguccaucaaugauagacuugauuaagaaaaugugcacauauacaccauggaauacuaugcagccauaaaaaaggaugaguucauguccuuuguagggacauggauaaagcuggaaaccaucauucugagcaaacuauugcaaggacagaaaaccaaacacugcauguucucacucauaggugggaauugaacaaugagaacacuuggacacaagguggggaacaccacacaccagggccugucaugggguggggggaguggggagggauagcauuaggagauauaccuaauguaaaugaugaguuaaugggugcagcacaccaacauggcacauguauacauauguagcaaaccugcacguugugcacauguacccuagaacuuaaaguauaauuaaaaaaaaaaagaaaacagaagcuauuuauaaagaaguuauuugcugaaauaaaugugaucuuucccauuaaaaaaauaaagaaauuuugggguaaaaaaacacaauauauuguauucuugaaaaauucuaagagaguggaugugaaguguucucaccacaaaagugauaacuaauugagguaaugcacauauuaauuagaaagauuuugucauuccacaauguauauauacuuaaaaauauguuauacacaauaaauacauacauuaaaaaauaaguaaaugua

SEQ ID NO: 228 - RNA, 1037 nt, Artificial Sequence
  Synthetic 3′ UTR-012 - Col6a1; collagen, type VI, alpha 1
cccacccugcacgccggcaccaaacccuguccucccaccccuccccacucaucacuaaacagaguaaaaugugaugcgaauuuucccgaccaaccugauucgcuagauuuuuuuuaaggaaaagcuuggaaagccaggacacaacgcugcugccugcuuugugcaggguccuccggggcucagcccugaguuggcaucaccugcgcagggcccucuggggcucagcccugagcuagugucaccugcacagggcccucugaggcucagcccugagcuggcgucaccugugcagggcccucuggggcucagcccugagcuggccucaccuggguuccccaccccgggcucuccugcccugcccuccugcccgcccucccuccugccugcgcagcuccuucccuaggcaccucugugcugcaucccaccagccugagcaagacgcccucucggggccugugccgcacuagccucccucuccucuguccccauagcugguuuuucccaccaauccucaccuaacaguuacuuuacaauuaaacucaaagcaagcucuucuccucagcuuggggcagccauuggccucugucucguuuugggaaaccaaggucaggaggccguugcagacauaaaucucggcgacucggccccgucuccugaggguccugcuggugaccggccuggaccuuggcccuacagcccuggaggccgcugcugaccagcacugaccccgaccucagagaguacucgcaggggcgcuggcugcacucaagacccucgagauuaacggugcuaaccccgucugcuccucccucccgcagagacuggggccuggacuggacaugagagccccuuggugccacagagggcugugucuuacuagaaacaacgcaaaccucuccuuccucagaauagugauguguucgacguuuuaucaaaggcccccuuucuauguucauguuaguuuugcuccuucuguguuuuuuucugaaccauauccauguugcugacuuuuccaaauaaagguuuucacuccucuc

SEQ ID NO: 229 - RNA, 577 nt, Artificial Sequence
  Synthetic 3′ UTR-013 - Calr; calreticulin
agaggccugccuccagggcuggacugaggccugagcgcuccugccgcagagcuggccgcgccaaauaaugucucugugagacucgagaacuuucauuuuuuuccaggcugguucggauuugggguggauuuugguuuuguuccccuccuccacucucccccacccccuccccgcccuuuuuuuuuuuuuuuuuuaaacugguauuuuaucuuugauucuccuucagcccucaccccugguucucaucuuucuugaucaacaucuuuucuugccucuguccccuucucucaucucuuagcuccccuccaaccuggggggcagugguguggagaagccacaggccugagauuucaucugcucuccuuccuggagcccagaggagggcagcagaaggggguggugucuccaaccccccagcacugaggaagaacggggcucuucucauuucaccccucccuuucuccccugcccccaggacugggccacuucuggguggggcagugggucccagauuggcucacacugagaauguaagaacuacaaacaaaauuucuauuaaauuaaauuuugugucucc

SEQ ID NO: 230 - RNA, 2212 nt, Artificial Sequence
  Synthetic 3′ UTR-014 - Colla1; collagen, type I, alpha 1
cucccuccaucccaaccuggcucccucccacccaaccaacuuuccccccaacccggaaacagacaagcaacccaaacugaacccccucaaaagccaaaaaaugggagacaauuucacauggacuuuggaaaauauuuuuuuccuuugcauucaucucucaaacuuaguuuuuaucuuugaccaaccgaacaugaccaaaaaccaaaagugcauucaaccuuaccaaaaaaaaaaaaaaaaaaagaauaaauaaauaacuuuuuaaaaaaggaagcuugguccacuugcuugaagacccaugcggggguaagucccuuucugcccguugggcuuaugaaaccccaaugcugcccuuucugcuccuuucuccacaccccccuuggggccuccccuccacuccuucccaaaucugucuccccagaagacacaggaaacaauguauugucugcccagcaaucaaaggcaaugcucaaacacccaaguggcccccacccucagcccgcuccugcccgcccagcacccccaggcccugggggaccugggguucucagacugccaaagaagccuugccaucuggcgcucccauggcucuugcaacaucuccccuucguuuuugagggggucaugccgggggagccaccagccccucacuggguucggaggagagucaggaagggccacgacaaagcagaaacaucggauuuggggaacgcgugucaaucccuugugccgcagggcugggcgggagagacuguucuguuccuuguguaacuguguugcugaaagacuaccucguucuugucuugaugugucaccggggcaacugccugggggcggggaugggggcaggguggaagcggcuccccauuuuauaccaaaggugcuacaucuaugugaugggugggguggggagggaaucacuggugcuauagaaauugagaugcccccccaggccagcaaauguuccuuuuuguucaaagucuauuuuuauuccuugauauuuuucuuuuuuuuuuuuuuuuuuuguggauggggacuugugaauuuuucuaaaggugcuauuuaacaugggaggagagcgugugcggcuccagcccagcccgcugcucacuuuccacccucucuccaccugccucuggcuucucaggccucugcucuccgaccucucuccucugaaacccuccuccacagcugcagcccauccucccggcucccuccuagucuguccugcguccucuguccccggguuucagagacaacuucccaaagcacaaagcaguuuuucccccuaggggugggaggaagcaaaagacucuguaccuauuuuguauguguauaauaauuugagauguuuuuaauuauuuugauugcuggaauaaagcauguggaaaugacccaaacauaauccgcaguggccuccuaauuuccuucuuuggaguugggggagggguagacauggggaaggggcuuuggggugaugggcuugccuuccauuccugcccuuucccuccccacuauucucuucuagaucccuccauaaccccacuccccuuucucucacccuucuuauaccgcaaaccuuucuacuuccucuuucauuuucuauucuugcaauuuccuugcaccuuuuccaaauccucuucuccccugcaauaccauacaggcaauccacgugcacaacacacacacacacucuucacaucugggguuguccaaaccucauacccacuccccuucaagcccauccacucuccacccccuggaugcccugcacuugguggcggugggaugcucauggauacugggagggugaggggaguggaacccgugaggaggaccugggggccucuccuugaacugacaugaagggucaucuggccucugcucccuucucacccacgcugaccuccugccgaaggagcaacgcaacaggagaggggucugcugagccuggcgagggucugggagggaccaggaggaaggcgugcucccugcucgcuguccuggcccugggggagugagggagacagacaccugggagagcuguggggaaggcacucgcaccgugcucuugggaaggaaggagaccuggcccugcucaccacggacugggugccucgaccuccugaauccccagaacacaaccccccugggcugggguggucuggggaaccaucgugcccccgccucccgccuacuccuuuuuaagcuu

SEQ ID NO: 231 - RNA, 729 nt, Artificial Sequence
  Synthetic 3′ UTR-015 - Plod1; procollagen- lysine, 2-oxoglutarate 5-dioxygenase 1
uuggccaggccugacccucuuggaccuuucuucuuugccgacaaccacugcccagcagccucugggaccucggggucccagggaacccaguccagccuccuggcuguugacuucccauugcucuuggagccaccaaucaaagagauucaaagagauuccugcaggccagaggcggaacacaccuuuauggcuggggcucuccgugguguucuggacccagccccuggagacaccauucacuuuuacugcuuuguagugacucgugcucuccaaccugucuuccugaaaaaccaaggcccccuucccccaccucuuccauggggugagacuugagcagaacaggggcuuccccaaguugcccagaaagacugucugggugagaagccauggccagagcuucucccaggcacagguguugcaccagggacuucugcuucaaguuuugggguaaagacaccuggaucagacuccaagggcugcccugagucugggacuucugccuccauggcuggucaugagagcaaaccguaguccccuggagacagcgacuccagagaaccucuugggagacagaagaggcaucugugcacagcucgaucuucuacuugccuguggggaggggagugacagguccacacaccacacugggucacccuguccuggaugccucugaagagagggacagaccgucagaaacuggagaguuucuauuaaaggucauuuaaacca

SEQ ID NO: 232 - RNA, 847 nt, Artificial Sequence
  Synthetic 3′ UTR-016 - Nucb1; nucleobindin 1
uccuccgggaccccagcccucaggauuccugaugcuccaaggcgacugaugggcgcuggaugaaguggcacagucagcuucccugggggcuggugucauguugggcuccuggggcgggggcacggccuggcauuucacgcauugcugccaccccagguccaccugucuccacuuucacagccuccaagucuguggcucuucccuucuguccuccgaggggcuugccuucucucguguccagugaggugcucagugaucggcuuaacuuagagaagcccgcccccuccccuucuccgucugucccaagagggucugcucugagccugcguuccuagguggcucggccucagcugccuggguuguggccgcccuagcauccuguaugcccacagcuacuggaauccccgcugcugcuccgggccaagcuucugguugauuaaugagggcaugggguggucccucaagaccuuccccuaccuuuuguggaaccagugaugccucaaagacaguguccccuccacagcugggugccaggggcaggggauccucaguauagccggugaacccugauaccaggagccugggccucccugaaccccuggcuuccagccaucucaucgccagccuccuccuggaccucuuggcccccagccccuuccccacacagccccagaagggucccagagcugaccccacuccaggaccuaggcccagccccucagccucaucuggagccccugaagaccagucccacccaccuuucuggccucaucugacacugcuccgcauccugcuguguguccuguuccauguuccgguuccauccaaauacacuuucuggaacaaa

SEQ ID NO: 233 - RNA, 110 nt, Artificial Sequence; identical to SEQ ID NO: 151
  Synthetic 3′ UTR-017 - alpha-globin-1

SEQ ID NO: 234 - RNA, 116 nt, Artificial Sequence
  Synthetic 3′ UTR-018 - Downstream UTR
uaauaggcuggagccucgguggccaugcuucuugccccuugggccuccccccagccccuccuccccuuccugcacccguacccccguggucuuugaauaaagucugagugggcggc

SEQ ID NO: 235 - RNA, 118 nt, Artificial Sequence
  Synthetic 3′ UTR-019  - Downstream UTR
ugauaauaggcuggagccucgguggccaugcuucuugccccuugggccuccccccagccccuccuccccuuccugcacccguacccccuggucuuugaauaaagucugagugggcggc

SEQ ID NO: 236 - RNA, 119 nt, Artificial Sequence; identical to SEQ ID NO: 150
  Synthetic 3′ UTR-020 - Downstream UTR

SEQ ID NO: 237 - RNA, 138 nt, Artificial Sequence
  Synthetic 3′ UTR-018 + miR-122-5p binding site, Downstream UTR
uaauaggcuggagccucgguggccaugcuucuugccccuugggccuccccccagccccuccuccccuuccugcacccguaccccccaaacaccauugucacacuccaguggucuuugaauaaagucugagugggcggc

SEQ ID NO: 238 - RNA, 138 nt, Artificial Sequence
  Synthetic 3′ UTR-018 + miR-122-3p binding site, Downstream UTR
uaauaggcuggagccucgguggccaugcuucuugccccuugggccuccccccagccccuccuccccuuccugcacccguacccccuauuuagugugauaauggcguuguggucuuugaauaaagucugagugggcggc

SEQ ID NO: 239 - RNA, 141 nt, Artificial Sequence
  Synthetic 3′ UTR-019 + miR-122-5p binding site, Downstream UTR
ugauaauaggcuggagccucgguggccaugcuucuugccccuugggccuccccccagccccuccuccccuuccugcacccguaccccccaaacaccauugucacacuccaguggucuuugaauaaagucugagugggcggc

SEQ ID NO: 240 - RNA, 133 nt, Artificial Sequence
  Synthetic 3′ UTR + miR-142-3p binding site, Downstream UTR
gcuggagccucgguggccaugcuucuugccccuugggccuccccccagccccuccuccccuuccugcacccguacccccuccauaaaguaggaaacacuacaguggucuuugaauaaagucugagugggcggc

SEQ ID NO: 241 - DNA, 29 bp, Artificial Sequence
  Synthetic T7 promoter sequence
  misc_feature (21)..(29): n is a, c, g, or t
taatacgactcactatagggnnnnnnnnn

SEQ ID NO: 242 - DNA, 18 bp, Artificial Sequence
  Synthetic T7 promoter
taatacgactcactatag

SEQ ID NO: 243 - DNA, 29 bp, Artificial Sequence
  Synthetic exemplary T7 promoter sequence
  misc_feature (20)..(29): n is a, c, g, or t
taatacgactcactataagnnnnnnnnnn

SEQ ID NO: 244 - DNA, 29 bp, Artificial Sequence
  Synthetic exemplary T7 promoter sequence
  misc_feature (1)..(10): n is a, c, g, or t
nnnnnnnnnncttatagtgagtcgtatta